SEQUENCE LISTING (126 sequences)

SEQ ID NO: 1 - DNA, 40979 bp, Homo sapiens
ccacaaatggtggggttacaggcgtgccactgtgcccatccagattcctgaaaatttaacaattttatgagttggtacatgctgactcgagcacacaccactgggaatagttgtgaggaggacagttgagtgctggggaaaggaaggaagaaaacagtgaggataaagttcacatatctcaccagcttttattacctgatccccatggggaggcccatcagagagtgcctatgacctgttacaatggactctaaaaacacttccctactctttcaagtctccctgtgagcattggttacacttccagtatcccattcttatagtttaactcatgaaaaagggcgggatcctccttctgccaatactagttccttctcctcaatgaaaagttagacacaaactccaaaataaaggcaactcccagaatacaacacagccccaattaaattaaaatggcttttatccaaaagacaggtaataacaaatgctgacaaggatgtggagaaaaagtacccttgtacactgttgttgggaatataagttagtacaaccactgtggagaatggtttgaaggttcctcaaaaaactaaaaatagagctaccatatgatccacaatctcactggtaggtatacacctaaaagaaaagaaatcagtatattgaagagatatctgtactcccatgtttattacagcactattcacaatagccaaggttggaagcgacctaagcgtctatcaccgatgagtggataaagaaaatgtggtacatatacacaatgaagtactattcagctaaaaaagaattagatcctgtcattcacaatgacatggatggaattgaagatcattatgttacgtgaaataagccaggcacagacagacaaactttgcacgttctcacttgcttgtgagaggtaaaaattaaaacaattgaacttgtgggcatagagagtagaaggatggttaccagaggctgaagggtagtgggggttggggaagaagtggggatggttaatgggtacaaaaaaatagaaagaatgaataagaactagtatttgataatacaacagtgtgactatagtcaataataatttaattgcacatttaaaaataaaaatataattgcactgtttgtaacacaaaggataaatatttgaggtgatggatatcccatttaccctgtgtgattattacatattgcatgcctctatcaacatatctcatataccccatacaaatatatgcataccccatacatatatatatacatacacacacacacacacacacacacacacacacacacacacacacatatatgtatatctactatgtacccacagacgttaaaaattagaggagaaaacacacacacaacaaggagactgagctggaaggatggagctctgggatagatttgtcctacatccctgcctgggagggaatccacacacatgcaagaagacaaactaggagcatgggctactaaattataccacattgcactcatcggggtcacagggtttcttccaagtgacccgcacatgcccttcccatctctgtgtgacagtggcacctgcaccagactgcatgttgaggtgtcatctgaaattatgaaataaaacagaagtaagaggtctattagctcatcaaaatgcagttatctaagttcagctgtgaactgccaaatttgaggagtgatccaatgaaacatcttttctttgcaatccaagaagacttaccggagagaactgctcagagaatctgcaacatccggttcctggagacagctaaggaaagaagctggggcgcatgtttctgcccaaagccgggttttggccgaggtgactacacaccccctttcctggctcccataggctaagtgcctggcttcttgagaagcctgcttcttgagaacaaaaaagtgatttaaagcctcatgggagatgagcaatcctcaagacacaagcagaaaaagtcccagtgatacaggaagcgggttcaggaacctgctggttcctgatacataaatcagacagcctctgctgcatgacacgaagcttgcttctgcctggcatctgtgagcagctgccaggctccggccaggatcccttccttctcctcattggctgatggatcccaaggggctcctctccttgaccttcgtgctgtttctctccctggcttttggggcaagctacggaacaggtgagtgttcatctgcctgatggtttgagtcccatgttagctgccaggaatcagcgtatcttcgtggatggagagaaggtgcagggctgggtattgtgtttggtcactcttccttagggactggctgtcagtttcaactgcctctttcaaagaggaaggaacattataagttcctgggcccttgggtttccaagactcagccccaccaaccccagtttccaaggaaatgaggggctctaagccaaaggctccagtcacttttctgaccagtcttagggtgacaggccctggtagaagtcttgcttgagtggttggttttacatgggcatcttctggcaaagacccagcctagagagactgagctggatggactgagctctgggagaagatttgccctacatccctgccctgggagggaatctgtgcacatgcaggctgacaaaccaggagcatgggtcaacagaaagcattggctagagtgggaagagagagtagaagtgaaaactccaggcttttggctgagaaccagcagtggccacagtgcggtcatactggtgtgtattttcttggaagagaaggtccaagaaagcaagagggaagaagttgggatttctgaaggctagggctggttacagtatgtgggaaatgcaaattgggaaccctcagagagtagctccagcaggaaggccagacaagagctacctttggatctggactctgttcctgtctttctgtctatcttcttcccaaggcaggctattgctttctgtttagaagtatcagggctatgagaaaaggtatttgagaaagaaaaagccaagcaagaagtggactttggactgcctgtgtgagtggggtgagaatctccttctgcttatttgtttagactgtgggaggtagcctggagtagaagaggtggcattacggacacggggggaaatcctgaggcccagggtgttttaagcttggggttttcaagaccgcaaatccaatatggacttttccaggaaaagcaccgtgatatgccagggatgtgggggtgctgcacaatggatgtgtcttttaccagacagccagacgaacagggcttgctcagcccactttcttttggaatctgcagatccatggctcgtacttcccaaggtctaggggaggaagaactgagctcggggctcagaaaaccaaatcgagccactttaagtggtcacagggaaagccaagcctccgttgttgcaaccaatttgtgactgcaccatttctggagcacctcttggtgactgtaaggtgtgatggagtgatggtgctgaactgtgaactggacttttccatctctgtgcttgctagcctcttggccagcctggcccatagcatcttaggcactgctgaccaatagctcgtcttattgaggctttggaagtcgccggtcagggagaagcaacccagccccacaaggcaagtctatccaatcggaggctgctcacttcattgcatgttttcttctttgaatcttcacaaaagtttttcagtgtttttatttttaaatacacacccttttgtgaagccccaataaaacccagacagaaatgctttgcaaatggggcaggttagtcatgacagatttgcccaagcaagaagcttgattcttgtaaaactggcatccactcccattctcatttctactcagctcaacttctaattcccagtcagaattgtaaaaatcaaaaagtccacatgtcccttccccaagtaaagtgaatttttcatttcccccgatgagatttgttttaatagactttattttttagagcagttttaggttcacagcaaagttgggcagaaagaacagagatttcccatatgccctctgccccatacatacatagcctcccctattatcaatatcccccaccaggatggtacatttgttataattgatgaccttatgtcaatgagttttttttattcctccatgcagttcctctgcacccccttcacacactttgggggaaggtgagggacaagtgcgtgggttctagacttggcctaaccttgcgtgttcagtggcccattctatcccaagatgtcaatctaggcacttctatttctcaaaaatattaatactgtgatgtgatgctgtcactttcactgtctccctcactggactggaaatcagagtatcagggctaattggtttaatccatagatttgactttgaatgatcccattcagcaactattaaggacacatgatgtggcagccactgtgcaagggtgcaaggtgtggggaaaacaaaatgaacaacagccactgcccagcacctgacactgtgtctgtactgagagcctgtccacaaatatttgttgagtgaataatactggtatatactgcatacttgccatataccaggcactgttctaaatgctttatgtgtgtcagtcatttcattttcacaccaaccctataagaaatgtatgtattattggtaccattacaactttataaatgaggaaaggggcacagagtagtttagcaatttctctgcgctcacatagctgcctgactccagaggcctcaatgagtaaaactggacaagcctatgtttgggaagcaggggtggagagaatgccaaaatttgtatccaggtcccatggtaaaaattagaatgtgctatctataattgaaaaatatgagttgaattgaattggaataaattgaagtatagaaatgtccaaaaggtgagagactgataaaaatcacagaagaacgtagagggcatataagaagtatttgacctggaatttggaaaatgaaaactttttttcatcatgcaaatgttcatttaatttttttttgttaactagtttgtttattgattatcacatctataaaacatgtcatgtctatgaaacttcaacagtacacaagagtatttagtgaagactaatttcccttccatacctcatgctggaggcaaccactgtgaccagtttcttgtgtgtctttccaaatgtataggtcttcgtgtttgatagatcagtacggtgactatagttaacaataatctattgtacatatcaaaataactggaagagaataattcaaatgttcttagcataaagaaaagagaaatactcaaggtgatacatatcccaattaacccgatttgatctttacacaacatgtgagtgtatcagacagcacatgtgccctgaaaatatatacatctattatgtatcaattttttaacatggcagagaagaaatcagagataaagagggtggggaaataaaacttctctcgacttttcagtgtcctggtgaagagtactagctctgacattttttcataccataagaattaaatctgtagttatttgcataggtaattgctctgatccaaacgaataataaaatttttcccgagaggagcaaatggttatagcctgaacaaggtccctaggtagagcgcccagggtgccatgaagcctggagtcactatcttcctaagcaggccagcataagcttgtgccatcattatgcagcatgcaagaaggaatgagccccagaacttggagtcaagtcccaggacttgccataaaagccaagacatgtaacggactatctggctcctggagagatttatctacctaccaaagtgttggaataaggagcagacctttaagacggggaggggggatagctgcctcctccctcttttataggtagggaaaataatttgtccttgtttcttacctatggagtgtctgtttactcacatagagcaattgaccttgctcttatcacatcatcctagggggaagtggggggccaaagcatttactatttactgtgagtcatttaataagaaatttaactctaatccagtatatctcatgtgcacatttgggataaagttaataaaaatgaatattaaaaacttagccccaaataaatgggtccgatgggcttgatttttatggaacttgagaaggagcgttctagaaggaggcacaaatgcagaggtaaagggtttcaagtgttcttggcaagttgtctgttgtacctgaaacctagggtttatatttaaggcacttccatgtcctcagctggcaagtggggaaaagggtccccaccactttcttccataatatacctcttagggatactataaaggcaaatcagagtacattctgcttttggagggaggagaacttggactctgtgttgtcatttgctcatttttcattcatcccattctgttttattaattcacttgggcaacaaggatttactgagctcctactatgtcccaggtgggtgttagggatactgtagtgaataaaacagacacagtccctactcttggaagcttataagagtgggggattacaggcattgaaccagagttgaacacgtgatgaatgatatgaaggagtgcgttcattgtccattggaagtctgtgacaggaaaacccaacctaagtcaggagtcaggaaagtcttccctatgaaagagatgtcaaaatggagaccagaaagatggaagttgttagctaggcaaataaagagtggttatagcctctaaggctagggaacgtacatactaatagtctaagatagaaagacccagcagtgccaaataataaaaataggactttgactgatggggatacagtttaaaaagcaaacacagacaagatgtcttttctttctgagcctaaatttaccaaaagaactgtggggtctgtaagttctgttccttctgtacctgaaagaatactgtagcaagatgctaaaagcacatatgaaagtgtcagggctaggcaaaaaaatataatacaataaaacaaaaagagttatcattaggtagaagcccatcttgtgagagggttggctaaatcctactattaataatttttgaccaaactccataggcccatgtgaatcactcatttttcgaagtagaattacccatgaaggaaagtgagttggtgttaacagctacaaatgtttcctcccagactcttttagtaaataagggctggctgaatcacagacacactggaaaacactcatctagcaggatgtttcaggagcagggacgccactcgaggggttttatgaaccactttaaagcccccacttatttttccaccttgtgcttatgtgagggtgatctcaagtaccccctccagaccccaacactcacacactcaggtattgcgtcatcattctttatgtgggttgtggggtataagggtctcttcctgatgaagttttggttccactcctcatgactgagtgtgcataaaaccactcagcctctctcatctacccctcccttttcctcttcctctttctccttctatgttctttcgtttattttatttttttatttttttatttttttggttattccctacctctcttatatccctctttctcctcccccaatcaactccaagttctgaaagcaaccatggcgcaaagagtgtgcaaggttaggtggggaaggagtgcatgggagccattttggggagtggtggcgatgggttatggcctgaaaatgggattttttattctttttttctccctatcaaagttggtctttaaaaatcaacactacgctagcaatttttaatcttgttttgaatctcagatccctttaagagatggcatttatggatgtgctcccagaaaaatatgtatacgctcatctatacaactttttatacaaaagtctggaagttcatacttgcacatatggctttaaattttttctcatttctttatacacagaagtttaggttcaggattcaagaagttactcttttaggtactgtgcctacaagtcaggtatgtagccaccaaaggggtcacattatctagacagtcaggcatccataagtgtggtggaagaaaatccaacatgcttcccagtatattaatgtaaaaacaaccaccaccaccacaataactataatgttcctcatgtcatcaagcagcaggggagagcactctgttttaagcttaatatattcgcaatattttaaaagacaaatgcctaattgcctttctcacttttcctcaacaattaagaatttcaatcactctaggccagattttagcccagatagacttctttttcttcttccccaatcactgaatctctagtctactattagctgagccctttactgagcaacatggggatttcggggtattttggtgacaagaatatttgggccagtgtgtccaattttccaatagctcatcttagccacaagtcagttgtgaaagagtctcttctaggtagctgcattacttaagctgatggttctattttactctctgactttcttatcagctagaacaatctatgctctctttgagtcatgggctccttcttttatgaacactagcttatggttaagttcagatatatatatatgtgtatgtatatatatatgtatgtgtgtgtgtatatatatatatgtgtgtgtgtgtgtgtatatatatatatatatatatatatatatatatatgacaaacctaataacctaaataagaggctttggtcaggtattatggttttcagcattcattcattgaacagatatttattaaatgcctcctatatactaagcacatagcacctgtttgtaggtcttggggtcaaaatagtgaacaaaatgaagttcttcctcttgaggcttttgcattctagtgggagagacaaaaataaaacaaacaaatatacagtataatataatgcagtgataagtgcagaaagaaacacaaagctattttagatagatggtcagaggaggcctcttggaggagaaactgttttgagcagatacctaaaataaagtgaaagaatgagctacccaggtatggaagggaagaaattcttcagagagaggaacagcaaaagcaaaagttctgagacaggaatgttcttggtgggtttaagaaacagccaggagccagtgtggccgtagcacagtgagcaaagaggagggcaggaaatggagttggaacagtgccacggactgggcatgcagggcctttgaagccatatcaataatggactatggttttattctatcggtgctagaaagccacagaaatttaaaagcaggagagagacaaaataggacatggtttttaaagatgattccatctattgtatgaatgcagggagatcagctggaagaagacggcagtacccaggcctgggatgatggtggtggaaatgcaggaggtgaaaagggttcagataccagacatattttgaagtcagagccaggaggatttgctgttaaaatgagtgtggagtatggctgggcacagtggctcatgcctgtaatcccagcactttgggaggccgaggcgggcagatcacttgaggtcaggagttcgaaaccagcctggccaacatggtgaaaccccgtctttactaaaaatacaaaaaattagcagtgcatggtggcaggcacctgtaatcccagctacccaggagactgaggcaggggacttgcttgagcccgggaggcagaggttgcagtgagccgagatcgcaacattgcactccagcctgggcgacagcacaagactccatctcacagaaaaaaaaaaattgagtttggagtatgcgagaaagaaaggaatcaaggatgtttccagtgttttggcctgacaaattggctgaattataatgtttgcagaaggtgttctggaaccaagagtttgtttgctaagtttgaaatgccctttagacctccaagtcctgtcttgtgtaggcagttgggagtgcagtgaaggttttggttgggagatataaccctgtagcatcccagaaatatgtcagactgtgcaattgggtgagaaactggatgagtgtggatgagaatgagaactccgagtactgagatgctccagtatttagaagtccagaagagcagaaggctcctgccaagaaaactgagcagaggcaacctatataggataggagaaaaaccgggagagtatgttgttccctgagccaaatgatgacagcgtttgaaggagggatggatgaactatgtcaagtacccctgagaaagcaagtaagataagaactttgacttggcttagtggagtagacagtgaccttgacaaaggtggttccagcgagcagtggggaagaacacctgtttatagtgggtccaaggaaaaatgggtctggaaatgggaaaagaaactataaacacacattaaagcactttgctgtaaaggaaaacagaaatggagaggtatctggggatggacctgggatcaggggagatagttttaatataaggaaactacaagtttatatgttgtgtattgatggaaataacctagtaaaaaaaaacctgataatgtgagggacagaggcaattgccgaaacaaagccttgaagtaggtgagtgctccgtggaggaagaggctcgacttaagtgggaatgtagaccatccatccaggtaggtaggttgatttagtggtggtaataagtggaagttctctttttgtgttttctattttatacttcagtgaaacaaaaagcaaagtcgtcacatgagagaggagggggaaaggcaggttgtgggtttgaggagagaggaggtgtgaaataatcagcagcaggaaccctcatagtggtttgaaaggctcttggtatttttttttttaaccttgttgttggctcagcttttttggaaaagagaaatacagtaatatctcactgtcgacattattaactatctcagggtgtttggagagagaggattccacagtttgaacactgggcttatcacttcctgactccacattcctcagatttttctgttttcctcatgatctgaaatgcttcctgggctcatgagctcagaatcacttttatttgctctccatccttcatcctgtatattcaatggtggaaaaaaccctggtagaggaattagcagaactgaattctaatcctgactctgccacttactagttagggaagccatttaacttctctgtgctttttagatgcctgaacaataaatctgagttgataaagacccagtactctagttaatctatacaactctatcctaagtaatttgaagatttctattgacagttttgaagtattgagaatatagtggggatcctcaaggcagttcttatagaccacgaaggacttggcaaccccagggatagccaaagaggaagagggagagcctccagtctgtccttcctgatctgctgacacgatgttgtctaaaggccttaataataagggactctcttctcctccctcccacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatggtaataatggcggcttccccagtccacactaaagggccaaggtgctcctttgaccaagaatttaggtctctcttaaaagcaaagggtattcagaattggaagtaactagaatgatcttctagtttgggggtatttaaacctgctgcatggaagacgttttaaaggttgacatttttttttccaaattgcatattgatggtagctgattaagcattagttactcttactcccatttcccaaaggaaaggggcacagctccttgtgggctggagggccgatagacccaaggatcttggtttgcaagtaatattttatttgaaaataggatttttttctgattaaaagaagttgaataccacagatcaaacccagtctctcctacatgaggacagtgaaatctaaccagaagcggttagcacatttacacacatttgtgtaggtgtttcactgcactgggggttctggataaagatggctaaaattcagcccacacaccacttgttaagccctgcattccggcaccagatcatacctacttggtggaagaagtgccttttggcatttaaacaaaggctttggttataaagtcttttagttgctgtacttaaactaggaaccaagtccacctgaatccaaggccagtgcttttttgagcctttttaactaccagtccctcttgagtgcacccagggattgtgtctcttaggcccagagactcatctgaattcccagggatcctgatagccacatggggctttcctgcttcttcaaaatgacttccttatctctggggatgggacaggaattcccacctaaccagcatttctttgaaattctcaaatatctagagggaaggcagcaatactctcaccaatcctccctcaacccagcattccctttccttcaaacagtgcctgcggaattcccatggccctcccccaggtacctgagagtcatttccagcagtggctccaggcacgactgccatgagcgtggaggctgcacatgatgcattttccaaaacggtgtggatgccagacattctgtcctttggttcctatgtttcctgtttttgtcacatcttgtgatcaaattcttactttggaaaatgtggtctctgcaaccatggcatttttctcaagccaaaggaagagtttggattttgaagtcagacagacctaggttcagatcttaacttggccacttagaagctgtgagttgtaagatattccacctccctgggacttggctttctcatctataaaatggggaataattacaactagagttataattgttgagaagattaaaaaagatgatgaggtggctcacgcctgtaatctcagcactttgggaggccgaggcgggcggatcacaaggtcaggagatcgagaccatcctggctatggtggtgaaaccccatctctactaaaaatacaaaaaaaaaaaaaattagctcagcatggtggtgggcacctgtagtcccagctactcgggaggctgaggcaggagaatggcgtgaacccaggaggtggagcttgcagtgagctgagattgtgccactgcactccagcttaggtgacagagcgagactctgtctcaaaaaaaaaaaaaaaaaagatgatgaatgtgaaacaccagcactgtgcttgtcctataatagttgctaaataagcaagaatttaccttttatgtggcctatttcatggccttagagtgggatagattgatgaggcctatggttataattgaggacctatcactatctcagacacacaaaagcacttactacacacccacccactcactcacccatgcgcccgtgtacatgcgcgcgcgcgcacgcacacacatacacacacacaccctcccacacacatcacgatagatgaaatcccaccactaaaaagccattcttttaggtctaggaagtaacaacgtaagccaactaaaaaccatggtggattagttgacagcaaactccactgataggagacaggagaatagcaacttaggtcaaggacatcaggaagggcgagtggagccctaacaatattggtagaagaggcctaaaaagcaaattcttattttctattttatcccaaggtggtcttagataggatgtagtggggcatgatggacagtgtgaagcaatagattccccactagaaataaatcacattgagggggagggaaaatgccattaggctgtactttgttctaacaaaaaggtcaagtgagaattcccaggggttcacttcagtgatggctcccttcctcccactcctgacagagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcaggtgagtacactggtggcagcctgtgtgccaccttaatgagcatgggctcagtcttcacatggtccaattgctccccagccatggcattcaccttagtaacaatactttacattttctttatagtttgcaaaagtttaccatgtgcatttagtgagctcactctcatattgaccagggtggcatttttatgctcagttcacagttgacgaaaccaacaggaggtgatagagacagacccagagcctaagtctccaaggcgtagccttctctgcctcttgccttgactccaacctgcaaggttggctgggtgagggattgcaggtgggagggcctggctcccaagtctatgctccactaatgctggggcagttctacaccgtcaattagtatttactgatcacctacctggtaagaaattgtgaataaatatgatatgtgatctctcctctctaacaattcattgtaaagataagacgtaaaacattgcttaataatgaaacacaggatatagtgaaggtcctcgttttatggaatggactgtgaatcccatagcagggccaggggagtcagaacgagagactaagacctggactgtgaagtttgaggaaaaataataaattgcacagcgcttcatcattagcaaagcactctcaactgtgtaatctcttttgatcctcacaataaccttgccagataggtgttatactttcattttacagatgaggaaattgacgttcataccatttaagtaccctcttcaagtttctttggcatgtagatagtggagttaacccctaaacctacatcttctgactgttcttcctctagaaagaaaagcatcattattctgtcagcaaaaggaagacagaacttactaagtaagcccatttaaccacttggatacaggacagaacacaggcccttttcactaacagatcttgtgtccctgcctcaggcaggttcagggactgtaggaccagggtgttgctctgcaaggcattgcttacaccctgacattctcctctctgcatccactggggggacataggaatcgttccaatgggtcttctgcctacagtagccatggtgtccatgtggagggctttcccaggtggtcatggttgagggacaggggaactcagccaagtaatgcccttccacatagcactgcctgtcacagagactcccctggagtattcccagatggactgctgggaaaatcccacctggcctccagtgtgcccctggaagttcttgaatgagtctaaccccctgcatgtttcctccccagacatttcatagccagagtcccccgtcctctcacttactggatatgtctggctgcttgcccaccttgcatacaaaccacattcagaggctaccccccagttcagagcgctctccccacccacagtcatttagaaggtgctggcaggacaagcaagctgtgcagcatagtgggtagcttacagaatgtgaccccggaagtcctagagccaatgctgccctttctacttacaagggcaagtgcctcaatccctctgagatttagattcttcatctctaaataacagcaactggcgggtcatcatggtttactatgtgccagttatgtggcatctcagttaatcctcacagcaacaaaataatattgatagtatccccacttcacagatgaggaaacaaagctataacatggttaagtaaggtcacacacagagcaagtgatggagctaggctttctgattctggaagcctcataggcattagtgaaagagataaagcacttaaaatgccttataaaccataataaaaatgtaatttttattataaaagctacaaaaatataatgtattttttaattgtaaatgaggaagcagtagcccttacctcagcacagccctctctgggtatagctgccctattagagtacaaaaacagggcactgaatattttaccctggcctaacccaaaaaaggggcagaactttcttcatgctcctcaatgtagtttaaaaagaatttaattagggcataccaaagtgttggtgggccataaagcttatactaagggcactcagctcaggacgttctaagaaaacatgtgaagatggacttacccatctaagccactctgaggaccaagaatgcaccagtgggaaccagatttactgagtaggaagctggttcttgtgaggagtggagggacaggaagcagtagaaacctggcaccacaggaagggccctgtcaggatctcggctcggtttgtaagagaactgtccagccgttcccctctcttgggtctctgtttcatccccagtaaaatgaaaagggcgaacaaatgaagtccctcccagagtagacagtccttgattcagtgtgtgtgtgtgtcaataagaactgccaatagaggcttgccactgtgtatgagtttgctaaggctgctgtaacaaaggaccacacactgagtggcttaaacaatcaaaatgtattggctctcagttctggaggctagaagtctaaagtcaaggtgtcagcaggattgttcttctctgagggctgtcagagaaggatctctcccaggcgcctctccctggcttgtaaatggctgtcttctccttgtctcttcatatcatcttctctctatgtgtatttctgtgtccaaatttcctcttctcataaggacaacagtcatattggattagggcccacctgtctcagtttgctagggctgccataataaagtacaacagattgggtggtttaaataacagaaatttatttttctcctggttctggaggctagaagtttgaggtcaaggtgttggcaggtttggtgtcttctgaggcctctcgccttagcttgcatatggctaccttctcactgtgttctcacctggtctttccttcgtgtactcacaccctgatctctctctctctctctttctctttcctggtgtctctttgtgtgtccagatttcctcttcttataaggacaccagttaggttagattaggggccaccctaatgacctcattttaacttaataacctttaaagaccttgcctccaaatacggtcatattctgaggtccttggggttagagcttcaacatagaaatttgggggaggggagacaaaattcagcccaaaacatctccctaatgacctcagttttactcaattaactctgtgaagacgctatgtccaaataaggaaatcaacatatgaatttgggaaaacacaattcaacatgtaataaaagtagagatgcctcctccccaccctgccagcctgcagggataggggaccagaccttccctgcctcagaaccagatcacacagctggtttgtggcctgccctgcgcagtataccagattgcctaaatactgaaaacagaattgtatcagtaccttgatttgtgtttgcgcatgaataacgaatactaccagttcttaaaacactgcacttagtttgacaaaacatttacactctacattgtgctaggggccagtacacaaagataaaaaagacatgatccttgccttaaggcagaagacagctgtataagtaaacaatgatccgagattagacagtgtgacggatgtaaaatggaaatatatgcaaggcaacaaacaggagggaattattaacactgtctcgggggattagagagagcatcccagagaatgtgacataaactgggttttaaaaataagtagaaattacccaagctgatgaaagacattccaggcagagggaggagcagatacagctgggtcttgctctgtattatcttgtaggtgataggaactaataaagagttttagatagaggagtgacaccatcagtcttgcttttcaaagaggaactccagtagtatagagaacagactggggcagggaagtggggaagaaagagaaaccagttcctgggggccattattgcagtcatccatcaaaatgatgggacccgagccaaagcagcaactgtcagttaacaaagacatttttcctagggcatacaaaggaaaaccccagccttgggatgaaagggtggggtccgagggtttattagaggttgctttgccatctgtcatcaggacagtgattttaagacatttttcattttcattaatgaaaacctcacagcggttaatggtgtgatgagacggaatgcaatgtgatgtgagcactgaatcttgacaggactcacttaagcgaaactgtgcaaaaacttatatgttccttgaaatctttttctttaggtgacatttgttcaggtcatgtattcatccttgttccaattgccatttcagtgtgttaatgtctatcataatgaagcatctttattgcaaagtccaattcttagggtgctatgaagtactctggctaggtcatgtgaagccagtggatgtgggtcagctgtggacagtgtgtgacttgctgccatcctcgatgactgtattctgaaatagatatggctgtgctagaatgaaggaatctagaaaggaatgcccctggaagctcatcttgaagagaggatctttttcagcagatcagcaaaccgctggctcagcacctctgagttagctcagtgaaagaaaaggctgacgcctgccagtgagctccggaggcttcccctttctaacaaggtcatttcttcaaatagggagttcccattgtttcagagtcacttagatgttccaggcactaagacaggtctctctctagggtcttcccaatttagcgagcgtaaaaacaatggtggaaaggaaaaacctggaaactttgcacagcccagagcctggtcatgggccacacccgctataagggaagctgagacacatagctcctagctgagcagctacatgcccagaaaagactcgtattaccacgaaagcatgagcgcaatctcactggagctagtagcctctgcaatgctgggtgggataggcaggttgtaagtgatttttctggaagctgtgaactccgtaaaaatgtttacttggatggtcccagaacttaaattagtatatggttcatgaggatccttccccacccccagttctgaatggaaactgccacgaacaagaatgtatctcttgaagatggcagcctttgctgacagaaccacatgaaaggcaggaaggagatccggcacgctcccaccgttacgctaacgtcgcagtatctcctaggtgaactgcatttgtttctcagattctttttagttttctttttcatcttccctaaaaaaaatattaataataagattttgggacttgagaagagagagagagagagagacacgcttctgtgtttctgtgacaacactttcagagacaagaaaaaaaacgccctctggctttttccttggatgtgtgactgtctgccaagttatcacgtttaaaccacagacaataggtggagagggcccagggtggagactcgagcaaagcactcttcccaaatggcatgtgagttattgaccagcctgctcggccgcctctaagagcctcgggagtagggggagttccaaacctctggttcagaaatgttcaggtagcatttctttgtgaatgaaggagtcaggagcttctagaccccaagacaactttgatttctcagcatcaccatccagagaggcctcactacatgactgagcaaagagaagaagagctggagcttctgccacaggaaatggtggtttgaaaatgggagcacaggtgaagcgccgatggcacagacacacacttgcctcctggctccatcttgttattgtaaagtataagccaagtgggtcacttctccttccctttgattcctgccttgggccattcagcaggtgaccctgcattccttctggtaatttttaaacagaaagctacgtgacagtctttttctagatccatttttgtggactctcatttaatttaacttagttcatcgagtgcatattgagtgccctcctgccctatattgtttccggtggaatggaggatacaaataaagaataaggtacagggcctaccttcatggaatttgcaatcaaagtgggacttctacatcttactagctagaaaaatataatatttaaagaaacatattataatcaaggaactgctactagaattcctctttgaaaaggaattgtatttgtttatgatagtaccttaataaatgctagaaggcaggtggagaccccccaggaatctgggtgtgggttggatggttctgtatgagaatggaggaagatgatacttgtgcagaaatgggaagagaaagagagagtctgaacctgctaggtggtgaaagctgcctggttcacaatggaatttgctccctgggacccttcaatcttcagcagagaacttaaacccacaaaattattggtgtaagtttttaaaaaaaagtttttttggtttgtttgtggaaactgattgtattagtccgctctcatcctgccaataaagacatacctgagactgggtgatttataaaggaaagaggtttaattggctcatagttccacatggctggagaggcctcacaatcatggttgaaggcgaatgaggagcaaaatcatgtcttacgtggcagcaggcaagagagtttgtgcaggggagctcccatttataaaaccatcagatcttgtgagacttattcactcccatgagaacaacatgggggaaaccaccccatgattcaattatctccacctggccccacccttgacacatggggattattacaattcaaggtgagatttgggtgggggcgtggccaaaccatatcactgatgaagtgactaaaccttgcacccaaggaagcacagagtagagcaagcagagttataggagcaaagacttagagaaccatgaggaaattactcccagaaattacagaaatcatgtgcagcttgacctgaacaaactgtaatagtagcacttttttcatacttatccaaatttctaagagcatggggtctctgacatttgatttccatgtaaatataattaaagaatagcaacaaatggatgagcaccaagtataaaaatacttgggcctactatacaggtagggaaactaagccataagtaaagaacagatgggactgaagcatctctggacactggtgaagagactcctttggacttaagatcaaactcattttcttgtctttccaatcaatcaacaagaatttactgagactctattatgtactgagtactaagagagctgttaaagtagtgtaagagatggtctctggcctcctagaacctagcaactatttggagaattgaggctagcagaagtaattgacacttactgaccacatgatggattccagatattgctctaggcactttccatacattactatatgggtttctcagaacaacactgtgaacttattgttatgctcattttacagatgaggaagttgaagccacagagggtatgagtagcttatctgtagtcacagagctatcaagtggtagacccagaatttgaacttatctatctggctccaaataccatcactgaaaatggtctgcatggtaaagatgatgttgccaaaactcaggttctaagatacatgacataaaccacaggtgctgcaggagtccaagggacagggaagaacaagagctgggatggtcaggaaaggtgacacaaagaaaggaagattggcctgggcatcaaggctaggcaggcatggtggacagcttagggtgcagcaggaaggagagtatggagtggagcctggggccaggaatgagcatgtgttggatggaggatgatgaaggatgggttggctgctcagagggcttttgactcaaaaggtttgagtcaaaagggctttgacatcactactgccttctttatggggaccgcatctccagaggctaaagcacaaaccacaaaatctgcagttcccatcttatccagctctgccactgactttttctatgactctggatactcctgtctgtgtctcagtgtcctcaaaataaaattagtgggttgggtgaaataagcactatactatagttcccttaaggttaaaaaggtctatgattcatatttgtatccaaagatgaggaaaaaaattagagtttatgaaatatctttcaggaccatggccaacttgtctctcagatctagatggactggcagaagcttgtcataggacaaaggtagcagattgctttcatcctctagagaccctagaaaagatagagagggccggcgttgtcatgtcctgaagccttggtgctgcacccagtcatcgttagtttctgtgagttggtgggcagcagagcaaccggcgtcgggcgcgagggagaggaggctgactcaccaggcattactggtgcagttttgtcttttattttccagttcagaggtactactttgtctgttggttttatttttttaatctcaagtgaaattggaaagaaatattatcttttaaaatgatataaatggtgggggtgtttcttctcaaatcagttgttgtattggaagttcccaaagtatctatgatagaagaagaaagaggaactagtcaaaatagtaagtgctactataatggtttgctggatcagttccataggctgacgaaacacaaagttcaggctactggctttgcttcttatcctagtattagagtgatttctccagtggttcctagtgtcgatatcataaaccttgaatgaatcaatctgtctcaaacacacacatacacacatacacacacacacacacacacacacacacactcctgcacagagggttctcagtgaccataagtcactcagagtggagctgctccttcctccagcatcagcaatgattcaaaatgtcatgctttatacaaattcagaactctctgcctgcctcctaacttttttttttaatcagagcataagactgttgaagttggtatctggcaaaattaaaacatttaatttaggggatagaacctataaccaaggtgtttgcaaagtcagttcagtgagattccttgggctaacttgatgtgtgaaaggcctaaggagaaaaagaatcttttcaaatccagaaggcaacttcttgccagctatcaggctggaggcccctttggatcttgtaggctgcattttatgaattcattgagactgtctgtatctttggtcaactctgtaaacatctgattgtgtccaccatgattctttcctttggaacccgactatttttctttcaatttctgccccacaaattcctcacaggttcaacaacaagcaggcttattccacaatcatccttataagtttcccttacacattaatgttaacatctggtgttactctatttagaaccttagtgcgaatattctacttagaaccctagggcttcagctcggtccccactgttcattaccccggtataactttttccaagcccataagtctctctaactctccaagaagtctgtctttagtattcagccacatttctactactaaaccaagctctagttcttgaggttctccaggctgttttccttctccataaaatgagaataatgagtgtacctaccttgtaagattattgtgaggattaaatatgttagtacacatgatgcactaaaaatatgtggcccattgcaagtgctcaataattgttcattataatcttattgagctacatgtcttgtttactgggggtgataattctcattcactgtttgtcaaagtgttgctcctagttcaaaaggatttgataaagtgggtaaaggagagaaaacaataaaagttttctctctgattttgagccttgatgattagttctcgggctaattttaaacatgaagatgatttagaggaaagactaaatactttcctttcagttcaggtctgctgggttcaacccagttatttgcatgaaaggacaacaatagcactattatgtttatttttaaaaaagataagtagatctttcttcctcccagtgtctcatgagaatagcgtgaattcacagggacggcacatggaaccattatattctctttacccaaaatggatacaggacacattagcaatcttaagatggagaaactgggcagagagattgacttaggagagatgaagataatttaatgttagacatgtggtagttgagttaaaaataaagcatttggatagaaaaatcttcatgaaattaagaatgtgaaagtatagtgagagaaattagaataagaaaacagatacaaaaattttcagtggtctaaagctgacctctaaaaccatgaaaacaaacgtctcccttgggagagaatgcagaaatagaacatgaggctccattatcccactttcatgtaagatgtttttaagctcagaatacttttgagattgctctttgacttctttttttttccagaaacaaaaccatgggcagtgtatgctgggctgttagggggtgtcatcatgattctcatcatggtggtaatactacagttgagaagaagaggtaggtgtctggcaataaatagattcttatcacactctctgtggtaagcaggggacctctctccacaggctcggacttgctctcacaactctggctttctgcatggggccacctttgcaaaaatagtagataaacatatcctgggaccttgcttaattcagtctaattcaacatgtcttgatcccctctactaggctgtggaaagaaatagaagagccacaggtttctaatgtgagagacattattcagataatttcagtttagtgtgactagcactgccatcagggtaaacacaggatgctgaagaagtgaacaagaggtttaagagtattcactgggaacagaattcagaaaattattggatctcatccaaaaagtcaccagggttagaatgaaaccaataaggcacaattattcccctgcagttgaagtgcctagaggtaccatcccctgtcctctcttccaaatttccctatgatacaatatctcagggcattgtgctcccctcagccaccttgactactaccaaccaatactggagtcaaaatgtcctgacccaagaccaggagagatgccccggctgccttcccatggtaaggatagaacttgatcctcataacactgagctgatgactgatttcattctcaagtagatcagtgtcatctacacacaaccttcttagaaaagcccttacctcagcactctgatgttggttttgcatatataaaaaaatctagatcatagcacagcgacctacttgtgtctcatttcctccatctaagagttagccaggtaggagggatgggtgattcagatagaaattaggttgacagcctatggggctcggggtagggcaatcacatttagctcatactataaggaaatagtgagatgacccaggatgagaaaactgaacttaacttatccacattaacctacctagtaaaattgctgggatcctacgccatactctttcctcaaccacacttggcttatcacatggttgtgctctaagggaatagtgctccccatcccacaattccccactaccttccccaacacacatacccatcctcacctcaaccccattcaccatttgtcccttgtaagttagcaacacacaaaactgcctcaaacttgcggtaaaatttatatttagttgctgcacctttcataaaaccttgctaaagaaattatattggcagcttctaatgctataatcatcagaatgcagcctgacgctgaaggcttttcaatttcatgactctttggcaatttcatgtccaggagaatacactgataaagaatgtgggtataggcattagacaaacttacattcagatgcagattttgctactgacaagctgtgtgatcaaatgacttaacttctcgtctgcaaaacaggggtaatactatgtacttcatggtattgtggtggagattggtatcaatacacagaaaacactgaacacagtggttcccatcgatgggtgatagatagatagatacatagacagatagatacatagacagatagacagatctcttagtgtagatgaattaaaatggcaatgtgtaagtgctatggccaggagaagctgcactggaagcatctggaaacaatacctagaacagattgaaaatattttaagtcatggtaacataagactttatgcttcaggtaaaagctgaaaaggatattagatactctatgccctcattttacagttatggtaagagaaaagacccattgagatgacgtgatttgtccaatgccacacagctaatgatggctacaatgtagatgtcctaattttaaggccaagactttttccttagagcctaagaccttgctgacttggagccgagttaagcttactcctaaaaacctgttcttgcactggggaaaataacctgagactaaattatcttggtccaatggtccttttaagcagcaacaatcaacctcacctcttccatctgtctgaccatttaggactgtccttccagttctacatttgactctgagctgacctgcaagactgaaagtctttgaggactgtagtctgttctctactctatttgtagccactacagcacctaggagagtgctgggcaggcatgtcttactttgcaaacactcgtggggactaacttgaacctcctctgctacctccaactgcttcttgagtcctcccctccattttacacacacacacacacacacacacacacacacgcactcacgcacactcctcagtcaggatcaactctgaccaaaaaagcgaagttgaaaccactaggcacaccgtgctcatacccacacacaaaaaatcccatgttgactttccttgaattcctggaacttcatcagtgtctgccccacatttcctccccaagactcacaccctcacgcagcacattccaccatgctcaccacatacacactgggcctttcccttccaaagaaaaatgtgcctctcctaaaaatgctatttcctcagagatgtgcctttttttttttttttttttttttgagatagattcttgctctgtcactcaggctggagtgcaatggcatgatctcggctcactgcaacctctgtctcctgggctcaagcagttcttctgtctcagcctcctgagtagctgaaattataagcgcgtgccaccatgcctggctaatttttgtatttttagtagagacagggtttccccatgttggccaggctggtctcaaactcctgacctcgtgatctgcccacctcagcctcccatagtgctgtgattataggcgtgagccactgcacccagcccagttttttaagagaataaattaactggtgttaaaataagtctaccttaaaggctgtgattttctgggtccagcctccattgcctctgcctggactttgcaataatcccataataaacctccatccttcagtctgccactttcccaccatccttactgctgcatgatgtatacaaaggatactgtgcaactttagaaagaatgagataggtctactgtgctaacatgaaaaatgtcctcaatacattttaagtgaaaagatcaagttacagagaagtgtgtgcagaatgacacctcttgtgtggaaaaaagtctatataagtatagcaaatatccaaaactgcattgtctaatatggtagtcactagccacatgtggctttttaaatttaaattaatttgaattaaataaaatttaaaattcagtgacattagtcacagttcaggtgctccatagccccgtgtctgtaagctgtattagacactgcagatatggaacatttccatcatctcagaaagttctgttgcacagagctgatctacagggatatacatcaaacttttaaaaatggtttcttcttttttttcccacttcttttcacaggtattgaaaaatacggtttcttttgggaatgaaattgggttggttaatggaagaaggggatttatactttttactttatactttatatatttcttcacaatttttattttatgatgagaataaattactcctataatttaaaaagaaagctttttaaaattggctaaaaattaaaatattctgcaacttattaatttccagagaccctaggccctgagcaaaatttccagatggtgggcaacagaatgacattgttgctttattttctaaatagtcccaggtggaacatccctcttacacgtccccccgcccttacctcccacacatcaattcccccagaaatagggaggtgagaaagctgtgagtgaagcaacatactaccagctggaaaatacaaaagaggtataaacaactagccctgccctcaaagaacttagaatcctattaggagaccagatatgcacattgagcaacagagattaaagtaattgaatgtacaccaatgagaaaaacacctaatgcgtattgggcatttgttatgcaccaggcagtgttctaaacactttacaagtggtatctcatttaattatcacaacagccccgtgaggcaggtatttcaaatcccatttcacagataggcctagagtgatcaagtaactaacctaagacaatatgacaaatgtgcaggggggctgggactcagggctttgtttccattgtgcccttggggaaagtgggtatgcaaaggacagtaaagaccaggtctgagtaaggagctcctgctggggaccagagggagataaccattatggtttcttttcaccaggtaaaacgaaccattaccagacaacagtggaaaaaaaaagccttacgatctatgcccaagtccagaaaccaggtgtaagttctatattttgtttgagatgaacctgtcatgtttcctagagtattcctggccagtctaccttgcctgttggacattcacagttttccatccagagcagaggaaggtagggaacaggagtcaagaacaagagttctcctaaagtcactaaacgtcagtgtttgaaataatgggcaacactggataattttctggtcatgagtcttcacaggaaaaaaatgaagaagctggaaatacatactgtatgactctttccagctctggcattgtaggagtctaggttccatgttagtcaattatttccttttctagggaaaagagtgcaggcttgaggagagaggaggtttggaaaagctattgtgtgacatgttggactgatccaagtttaggatttactaagtgcaaaagtgacaaggaaggtaggatcttcaaaattctagctagagtgtggttaaagagatgaaagatgagatggaagaaagaaaactgtgacagagtgatcactggactaagaagtgaaggatggaaaaactggatgcatggtgaagttgagaagcagatatgcttgaaggaagggatagagacgctaaaaggatcgtggttagatgtagagacactgtagtttttcaacatgaaggcaattcttggtattgtataggccagaatctggacatttggggtgtaggtagaggcaaattcttgagtaaaggatgtgaaggtaaagatggttttgatagtaccttagaaaattgcatgaaaagacagcaaatgcacttctgagaaccaggagatggactcttgaacaaagttcttatttctgctgtcccctagtggcctggagggcttattacacaacccagctccatccttcccccaactaaactccatttaaatagatgagaatcccaagagtaaccctttcaccccacgctctcatctgcctgtttaggtaaccaggttcaccttgaccatagtgtcttccctcactactctatcctatgctgctagcatccctcttttttactgtgaagcatgacatatggtagtcactagccacatgtagctttttaaatttaaattaatttgaattaaataaaatttaaaattcagtggcattcatcagttcaggactgtcctcccagttctacatttgagtctgagctgacctgcaagactgaaagtctttgaggactggagtctgttctctactctatttgtagccactatacacctaggagagtgctgggcaggcatgtcttactttgcaaacactcgaggggactaacttccacctcctctgctacttccagctgcttctaatcacacttttagtcctctcctccattttacacacacacacacacacactcactctcacatacacacactcatgcatacccactcctcagtaaggatcaactctgaccaaaaaaatacacaacacattaatgtcagctcagtgagttacccttaaacacatatctcgatatttggtaaagcaagtcttcctaatttgtttttctgcaaaagtttttggctattcttgttcctttatactttcatatgtattttagaatcaacttatcaagtaccacaaaaagaaaaaaaaatattagaattgtattgagtctacagatctatatgaggagaaattacatttttcagtgttgcgtgttttttgttttttgttttttgttttttgacagagtcttgctttatcgcccaggctggagtgcagtggtgtgatctgggctcactacaacctccgcctcctgggttcaagtgattctcctgcctcagccttccaagtagctgagattacaggcacctgccaccacacccagctaatttttgtatctttagtagagatggggtttcaccatgttggccaggctggtctcaaactactaacctcaagtgatctgcccacctcagcctcccaaagtgctgggattacagatgtgagccactgtgcctggcctcagtattgagtcttctaataccataaaactaccactcagatcaaagactagaacattgcccgtacttcctgaaggcctcctgtgccacttcccaatcattacttcctctctcctccccaaagataaccactatcctgacttctagaaaaataggttagctttttccttttttatttttgaactttataaaaattgaattctttattcttttttctctcatgtctgatttattttgctcagtattatctttatgagattcatatatgtctttgaatttagatataatgcattctttttcattgcttcatagaatataaacgtatgaatatactagagtttatttatccagttgactattgatggacatgtgggttatttccagtttgaggctattatgaaagttgcagctgtgaacattcatatgcaagtcgttaagtggacatgtgcacatatttttttgggtatatacctagatatacctggaagtagaactgctgaatcgtagagtatgcatacctccaaattgactagataaggccgagctgtttttcaaagtgggcgtatccatttacttttctatcagctacatatgagagtctcaattgctatgccttttttttttaaatttttttttgagacagagtttcactctgttgcctaggctggggtgcagtggcgtgatcttgtcttactgcaacctccgcctcctgggttcaagccattctcctgtctcggcctcccaagcagctgggattacaggtacgcaccaccacacctggctcattgttgtatttttagtagagacagtatttcaccatgttggccagggtggtctcgaactcctgatctcaggagatctgcccgcctcagcatcccaaagtgctgggattacaggcatgagccaccactcctggcctcaattgctatgcattctaatgaaaacttggtattaacagtctaattttagtcctactgttggatgtgtcttattatgcttttattccacatctctgtaattattaaggaagttgaacaacttttcatatgtttattggccatattaaaattctttcttaaagtgcccatttaatctcttgcccatttccctttgaggtttagtctttcttttatggactagtatatgcttttcatatattttggatatgtgccctttggcagatatgttagcaaataccttcacccatctgtagcttgcctttggaatttctcagagatacctactgataaagagaaggtcttaattttgttgtagaccaatttagtctagtcctttttaagcattactggattttatttgctaatattttgttaagagtttggttttccacttatgtttctgagtgaaattggcctgtaattctcttgtataatgcctttttttttttaagaaggcactgcagtggctggtatatagcattcttgtgaatatatctaactgggatacaagttgaggtagaaatatttcaaatgtccttaaaaaaataagtaacagagttcttcctggactcttctttaatcacaagcctcagattgatcccaaaatgacacacagctactctacctaatacccacatcacggtaaagttggtcgctctcctgttaaaaattcagactttaagaactggaagggacctgggtagtcatgcccaaccagtggggttttgatataaagatttatgctaattcacataaggagttggggtatatgttagtttcctagggatgccttaacaaattactggaaacttggtggcttaaaacaacagaaatttattctctaacagttctggaggtcagaagtccaaaatcaaggaggcacatcctcagggccacactccatctggaggctttaggagaaaatcctctttgcctcttccagcatctggtggctccaggctctccgtggcatttgttggcttgtagttgtgtatctgcaatttttgccttcatcttcacatgacctccctctctgtgtcttcttcttttccatctcttataaggacagtcatcatcagacttcggacttattctaatccaggatgaccttattttgaaatccttatcttgacatctgtgaagacctttattcaaataaagtcacattttgacattctgcctggacatatcttttggggccacagttcaacccaccacagggtgcatttcctttttgttattctctgcgatatttgggtaggatgtcttatttctccccttaaatatttgctagtagagcaaattgctagtaaagctatctgagactggggtttctttggtggaaatttttttaagttatatttttattattaaattttctcctctacatattagtgaacaacttttttcagtctttttagtggctaccctagaaattataaaatacaactttgacttaccaaagtctaaggtttacttccctcctgcataatacttcaagtccacaataatttcacctcttgatttaagtgaccgttttgtcattatttgaatttcatatatattttaaacacacaagacataattattattgttttatatatataaatatatacttagacttacccacattttcacaattttctttgttcatatttgcgatgtctttattatatcaatataaagactgtaataatgtagacaattatttaaaaactaacaatgcctttattcttatttttaatggctataaaataatcttataaagaatataataacatgaaaatcactaaacaagtgtttactgtgtgctaggaactcttctaggacttatcagagctagtatcttgcagaattaattccagcggccaccattcacaaaaattatgtgaaaataatgcctctggagttgcttgtaaatgatgctccctaaagatgtacaaatcagtggtcctaacagaagataataagatacaaaaatatactaacttattatatttatgtttaaaataattccctatgcctggataaaaatcctgaagtgaacatttaagcacacacagagtcttaataggactatgggtgacttcttttacatatttttctcctttctaaaacttctgaattaatgttaaaaatgtaagttatttgcctccttctgcctctaggtcaggttatgctaaagttctcccaaacaggaagaccagcagaggttgcatctgttgataaaggtctctcttcttttttttttttttttggtgatgcggagtctcactctgtcgccaggctagagtgctgtggcgccatctcagctcactgcaacctccagctccctggttcaagggattctcctgcctccgcctcctgagtagctgggattacgggcatgcaccatcattcctggctaatttttgtatttttagtagagatggggtttcactatgttggccaggatggtctcgatctcctgacctcgtgatccacccacctcagcctcccaaagtgctgggattacaggcgtgagccaccacgcccggccaaaggtctcttaataaactgttttgatagcctctttatctcatcactgcaagaaattctttcttaaactcaaaatttcttcaaaatgtatttaagaattgtatgggatcttgaaagccatctatctgaaccacccaattactgcttgaattatctgctacaacattacttccaaagtgttgcctagctcctttttactgaaatatagtttgtgaacaagcagcatcagcatcacctgggactttattagaaatgcagaatcaggccctgctccagatcttccgaaatagaatcaaccctttaacaagatccccaagtaattcatatgcataataaaagtcagcagcactggtctagaccatgcccaagcacttataactataggagctcattgcctgccaaggcaattcatcccacatttgaacatcttttaccattagaaagatcttcattatattgaatcaaaatattttccccaaatcctaatcttggtttaaacctgagatactttataggcaaattgaattccttttctatatggcaattcatcaaatatatgaagagaaaaattatgtcccatccctttttcctccaataaactttcctaattccttaacccttccatcacatgacaaaattccaagttttctcgcattaaaacacatgtggtgtggcttcaagtctggctctatacccagggagagtggacagcagcattatcccataaccagtgtccccaaaatgtgttgaattaatgacttccctattgtaagtgatggcatccgcatcttacaaggatgtggtctcaatttattttgaggtctttgtccaggaattgtggattttaattcgttcaaagtaacatcaacaaatatcagctgaagagtttatttttatgtgtccaatactgttctgtggggagtacaaaaatatatggcttatttctcaaggaatgtatagactggagaaacaacacataaatatatcagaatgttttaaatatagcatagagtgccataaagtgtaaattagactttaaatacctgaggaatttaagtaagggataggtcattataagttgagtgaccaaaaaagaatagtgatagaaaggacatgcaccttaagttaaaagagcttaaggcaggggactcccggctttgacacttcttgcttgtacacattaggaaaacatttggtctttctgagctgtagcttcatcatcggtgaagtgtgagtaacaacagtatttaacacagagtggttgtgaggcaaatgagaagacatatgtgaggaggaggaagagtaggaatggcagtgtgggatgcaagaattctgcatgtagccggtgatatgagtgagacggactaaattctgttgctattctgtcccctccagctgcccctgtaagagccacaccaatttcagtttcttgtgaggaagacatttaaaacatttgagaagcactgacaatggatgaggctgccttgggaggttgtgcaccccaagacgccacttggggagtccaagcaaagcctggggaattgagttccagagaatttgggggagaattcccacctgagaaggaggttggaccaaatgactggaaggaactttctgcctcaagtcttcttgagtctgtgcttctctatcggagagttgggtgagaactagctctctctgttcagctaatctgctttctttgcttctcttgtagcctcttcagaagaaacttgactccttcccagctcaggacccttgcaccaccatatatgttgctgccacagagcctgtcccagagtctgtccaggtgaggcatctctctgcctactctccgtagagagggaatacatgaaggaggggaaaatgaggaagttttttttttttaaggtgggaagagggagaggatcagggaaaatagctattgggcactaggcttcatacctaggtgacaaaatactgtgtataacaaatcctcatgacacaagtttacctatgtaacaaaccagcacatgtacgcctgaacttaaaataaaagtaaaaaaaaaaaaattaaaacaaaaacaaattaaatgaaacagattgatgagtcctggactggggaagggaggccacagcatgcaggcaaaaaggagtctctgtggctttggttttccagtttccatgaagcccccaatacctgctcacacggggccactgctaaccccctgctggccagtgtttccctgagagttgtccaaggaccacatcagaatcagccagcgtacttgttaaaaataaagattcctagggacttccacctaggattctgttaaatgaaaatgtctatggagagtagccatagacctacgtatttaaaaaacccacaccccaggtaattctgatacacactcaagtttaagaacagcagctggagtccaggagttctcaactccagctacaaaacagaatcaccagggaagcattgtaaaaatgctcatgcctagactctgtgcagccccatttaatcagaatatttaggggtggagatctgcataggtgttaagcctagaagagaatatggggtgcagctcaaaatgatacttgcatattctaccctattgcaagatcagcagggactaagtttacttcggacaggaatctttcctttactgaatgaatagaaataaattctgggctgaaatctttgctccatttgggctctttcagaagagagcccaggatgatagaggcacaaaggtcacacaaatgcctgcatccaccttatttttcaaagctcctaccgcacacacactcatccagaaatgcctgggcaggtgccctatatttcaagatgaaaccaatcttcaacttgaggtccattctcacttcactgtcatatctaagaaggaagtaaaaatataaacctgacttcaaagcttcaaaaaaatacatagatttttaatgaagtttacttaaggacaaaaacagtatgctatagttaacattttatggcaaaacccttaaattctattttctttgtttctttgacatgagagatctttgcgcataaccctcttctccccttcctctctcctgccaataccacttttctcttctccctttgagtcccactagactttttaaaaactcaataatttacaactctcttggcttcccagattgtgacccatatgtaacagcaaaacaaatggttttccttacaaggggatggaaggggagagggcaaagagggagacagggcactgagtgctggtcctcagatcatgctccccataatagcatgcttatgcttggaagggagctgtggcccttgttgcaggtggagaagcagtgtgggaacccaagtgctgtcccagcaaggccctgtctgtgacagaccctgcacaagccatgatctctaagaccctttccttttcctcagcagtgctgttttcatttgcattctgtgaagtgagtatccagtccctctactcacagacttctgctttgtccccaggaaacaaattccatcacagtctatgctagtgtgacacttccagagagctgacaccagagaccaacaaagggactttctgaaggaaaatggaaaaaccaaaatgaacactgaacttggccacaggcccaagtttcctctggcagacatgctgcacgtctgtacccttctcagatcaactccctggtgatgtttcttccacatacatctgtgaaatgaacaaggaagtgaggcttcccaagaatttagcttgctgtgcagtggctgcaggcgcagaacagagcgttacttgataacagcgttccatctttgtgttgtagcagatgaaatggacagtaatgtgagttcagactttgggcatcttgctcttggctggaactggataataaaaatcagactgaaagccaggacatctgagtacctatctcacacactggaccaccagtcacaaagtctggaaaagtttacattttggctatctttactttgttctgggagctgatcatgataacctgcagacctgatcaagcctctgtgcctcagtttctctctcaggataaagagtgaatagaggctgaagggtgaatttcttattatacataaaacactctgatattattgtataaaggaagctaagaatattattttatttgcaaaacccagaagctaaaaagtcaataaacagaaagaatgattttgagatctctgagttttgaacagtggactggaaaccatgtaagagccttaaaagtacagttctgtgcaaatggcattcagttttaaagaaaaacgtagcaaatgtttgatggtgctgttacaaaggagcttggaatactcagaggaacttgtcccatggtgatttttcacttctcaaaatgatgtttaaatcccagttctctgttgattcccttgaacaacaaacctggaacctcagctaagactctctgtgaccagattctgaacctcttatatccagggcttcaaggggtattgcaggtcaaggtctttcctaggcactttctactccctgcatacctctcctcacactaaatttatcctctagtagaaaattaagttattttggtctaacagcttcaaatctttgaatgctcaataacttattttgcaagctgcaggcagaaagagactttttaagtaaagtcctttgttttttcctattctctgcttttagacaggctgtcctcaatttaagccctgctttttcttattgtttcttatataaacttggtaagtactgtaagaaacagccactatcataccattgcataataaggagcaccaacttcccagctcaaaactcaggtccttattgccttgtatcttacctcctctatgaggtcaattcacattgtaagcctgttgcttagtgcatctcgtttcctggtaccagcttctttaatagagttcttagttgcaatcaacagaagctggctttggcttttttatgtagaaaaggaacctattgaaaagatactgattggttccaataactgctagaagtttctgcaaaaccatgctttgaaagtgagcaggaaaagaagagactaggctgtggctgggagcacagccaaaattacaaaaccagcccagggatgatgatcctgttcatgcacagccactgtccccagcactaggcacagactctaccactgcctcactgtctctgctggacttggaaacttgatattactgttactgctgcactgtctgccatgaaaatgaattctccagggtcccttcttcatcctttcatctctagcttataattcaaagtctgggattgagtggccaatcctaggtcacatgtccatgtcctatctccaaggggggctgggaattgaatatctggcattttccactttcacttcttatgaattaaggaattctacaaataatagaagtgggattcaggtggtaggcagacaaaaaagcctcacaattatccactacgccacccttgtataaccttaccctcattcactgtctactctcaaaactgtggagctactaatgaagatttgtaaacccgggcttatgagcacccattcctttactacaactcagattgctctagaagctcagttcccagcacttggatttttccagtagctgaattctacctgaaggaagggcagaaacaaagggtgaagaagaggctatcacttccaagtatcctgcacccctgggctcaagacctcactggggagggagtcttttgggccacccaccaaacagcactggcattatgcctctcaccctagaccatggttacacgtggtaaaacaaccccttctggtgatacattcacaactctctagtttcccccaaatggcactatggggagcgggagcttgccttttcctcagacttaaaacaataagttttccccgtgtttcccctctaatgctgttttcttttgaccaagcatgtctgaattctagagaagtcaggaggaacacacccattctcggtttgaagggactgatgttctgaagtacaactgggcacagtcccaggctcttcaggacgcttcctccattcacacagcggggatgtgattgttacagcgggtggtgtgtgctggctgagaagccactgtgaattgattcttcttctgaagtttatgtttctactttttggaaatgaataaattacagccagtccatcaaggaaattgcaat

SEQ ID NO: 2 - DNA, 42112 bp, Homo sapiens
gcaagagcactgtccctggctgtggtgttgtttctctagtcagttcccctttctgtatttgagttctaccgtcagtcctggcattatttctctctctacaaggagccttaggaggtacggggagctcgcaaatactccttttggtttattcttaccaccttgcttctgtgttccttgggaatgctgctgtgcttatgcatctggtctctttttggagctacagtggacaggcatttgtgacaggtatgtttgtggaggctcagacgcctagggagtggcatgagataaagctgcaagctgcatctggggcagaaatgctgatgtgctaatggccggccagagaatgagtaaaagggattgcagagagcatgcttaaaacctctgaccatcaggtttgcttctcagattgactacattggaggtgggatattacaaaaatctgtctcttcctgccagatcccttcatctgtttttcgtgagctaagagacaaaataggcaggaaatagaaggtgccacttaccaaataattggcagctgttcttggctttggggtgctggggtctccgagcagcctctgctctagaagaagcagtccaaagatgtcagctcgcctcgcctgagtcccctgtgccagtgggaaatccagagaagggggatttcctcctcttgcagcctctctgcaatggacttacttggctttcctgtttgacctttcccttctctggtccagagacccttccccaatatttcttcccatccaagtgccccatcccaatattagccccacttggcaccagagaccaagatctaatttaaaaagaaatattcttgggtcaaaaaagagcccaagcaagtgattgaacataatgtgtttcacatacggtgaacctatttgcatttgcatttgcaaacgggcttaaaatatcatctctattaatagcaatttaaggttctggagagccaggtgaaaatagtttttgacaaagggaacttcctactccccttaaactgtaataatgaaggaaatgaactgtttatcttacatgtaacctcaatcttgggactaaggccctgtactaaaatgcgtctatttatgtgctcagacttgcagttcgtgttatgtctgctgctgcagataccgttaatattatttatgtgagctatcctgtgtataatggaagcttttataaatctctatttatttattcctaatatagttattaagtgcttgctatgttccaggtactagggacttaacaggtagcataaaagacataaggaaaagctgcactcttgttttctagcctagtggggaaatcacattaatttaatcacactaaacatgactacatagcaatagtgctttaaagggaaggaaattgttctatgtgactatatcagctgattaattaccaagcctttgcatttgatattttggttagtctattcttcttgaatttcatatgcctcttcctgggtgggggtgaggatgggattttatggagttgaggctagggcaggtagggagaaaacatgagaaagatgaagagataagccaagccagattcttcagcagaaaaatcaaggttgaaataccatgtttcaaaaatcagactgaggtgggagttgaggttaggggtccctaggccaggggattgaagcttcaaagagataaaactagagcaaaagcaagcacagagagtggcagagaggtccctgggcatttttccacagtccattctagtgctggcaatccacctttcatggccaggcaggtaagagtatttgtggggtgggagaaaggacagggccataggctgggcacacagccctttactggcccttatctctcctctcttctcctatacagtgctgtttccgaactgtacattggcttacactcgggctgaggtttgggaaataggcgccattttgaatatgtgtggaggaagaaaagtgtgtcttcagcactttccacctccccatcacggccctgagacctcaacaccgggaagcatctcgttccctatcggtcctcctttattcatggacggatatgattcctttctaagttccatgtcctttttagataaattaacttgaacctaatgcctaatggcttaaaaacaaacaaaaaaaaccctcttccttccagctagcatttgcattttaacaggggctttcaaaaaatgccttagcccaaggaatgagtaatgtgggaattccaagcagcagggtaggactggtgcacagtatggggagagaaggcccctcaagttgtggccctgaaatgttggcttcctctctttgaccatgatgctgtttctgagaaaacaagaatcaggctaccttaggggaccaggatgggcatggctcccttttagtgagttctatgagcctcatacctgacagtcagagccctcgagtggatgagcacagactagaagaagcactgtgaaactttgcatgatccttacctttttggcaaaaaggaaaaaaaatcgttctcaaattcatcaatagtttgaaatagggtgtgccttgattcagaaagtttcgattctagatacaactcggagaactaggcgtgtcttgtacacagatttgctcttgggggaccggaaaagctaaatgctatcgccatgctatgctccttcttctaggccagtgaggggaacgcattcttcattttaatatttcagttgcctacaatattggaaggtggataaaagcaccctctgctccttctaaatctgcgaagacatttcttctctgcacctactcatccttgatgcagcttcctcatgtctgtatggaaacactgtgctctcaaatgagtttcagaaagaacaactcacgaaagaaaacaagcattcggtcagaaaaatctccacaaatggggaataagggggatttgctccaaggagagactggaaaccaagtcagacataaaatccagcctaagctagaaggagacatggctggtgggagcttgaggaaaacagagctcaggatggaggacgtctccacctccagtcatgtcctctgtccaccagacaccaagaagtgttcatgttccatcgaggcagccctcacacccatcccttcctcatcatgccgactgcctctttactgcttcaggctcacatctcaagtcgacgagcctgtaatactggctttcttgatcaccctgataccagccgtcacctcttgacaggcttattttctttaagctgtcattacaccatttttctgctcccaaactattaattccaaacttccaattttctgttaaattaaatatgaattccttatttgactttccatgccctattaggctatcttgctccttgctttacttatagaaactaatctcccattatttatccaaagacaacctctgctgcaggccagtcagcttttcttactgtcctgtaaaaattccatggtcactcctccatttccatgtgtccttaaaaactgttatttgattgtgtctcagaaagtcgtcaaagaatatataccaatgaaaagcatcaaaaaggttatacttgatgttatgtgtgtatcaaaaatatggctgaaatatttatccagtgaaactcaatcaacactaaaaagtggttctttcggaagcatcagttctttgagacccattaaacagatgcctcggatgcagggttatatattatcaggaatctgtctagggaagaattattggaagcttgcaaagcctttcaaggacagaggacgatagctaccacgttgagttctaggaaattaaccattgttattgttaaaggaagacagcgtttctcagaggaagactgttaaacagtgcagtggcccaggctaacagccctcataagtgggagtatcagaatgagtggacttaattacttaaaaccaatacagggtggaacttcatctgctataacagaaatcaactcgtgcaagttctaacatgcagggtacagttctgagaccaagtctgactcacctgtcaaagctcagctcaactattaccacctttacaccacccttccaagctgtaggagtgcttgctgttctccatgtcttctgaagccctggatcacttgtagccagctcagcagactctacccagacagggatcctttaaatgtaccatattgtctactgtgttaaaaatgagaggaactgactcagggtgagagcgatggagtgtccagatgttctcctttatttctccttattcctggaaatgtaatgagaatcttagaggtgaactgaaaagttatgagttcaaccacttactcaattcgagattcgctcctaaaatgtctcttctgtgttatcacccccactttggtttgaatagtacttgtgacagggagcttatcacctcacaagaaaatccagtcattgcttgtagctctctattaaaagttttccatcatctggaactgaaatctggctccctgtaacttttagttattggaactacttgcccttcagcaacagtgtatgtatcctcccatggaagggcccttacatatttgcagacacccagcatatacttgcaatcttttcttcttcaggttcattaccctagtccttttagttgttcttcatttgacataatttcattattcactagtgaaccttgctgcccttccccttgataaaccgaatttgtcagtgtcattcaagtataactgacctcacagaacgtgataccacaagcgatgtggtctgattagcacagagttcagtgaatgaatcctacactaggattggatgaaatttacttagccataccacactaacacttatgtgatttttatgtttactatggatagactatttctcctgtgtccacttcttcctcttacacagttgttatttcaaaactgaagtacagattcttacacttaccctcaggagattcatcatgttagtattagtctctcttttcaggctttatgaatgttaattcagctaactcatttttgagctatctgtctcattttgtgccatctgcacagcataagtttgatttctgttgcttttattagtagttttactaaatacataaaagtgaaatagtgaaacacagagtcttgtagcatccactgtgggatcagtcttttagacaagaatgatgcagttgctgagtcaaatgaataaatgaataaatcaaacaatactttgtcctcatttcccatattgatctatcaccatatcctgttaattataattctaaatatttcttgatctatccacttttcccttacttcacctgctactatcccagaccaaacagccatcttctttcactcaaacaattgcagtagccaactgattggtcttcctgcatctgtcctggcttccctatcatccatttgctacacagaaaccatggtcatcttttcaaaatgcaaatctgatgatatcagtctcagctctaatttctttggtggttcacatataaagactgaaatctttaactgaccaataacacacgtgtgatctggcccctgctcacctcttcagccttgtctttcacctgtctcttcattttggccacagggacctcctcgtaccttctctcacgtgccctcctgcctcagcgcctttgcatatgctgttccctttgccgagaactcttcctgtcaactcccaagcccttcacctacttagcacctacctattcaatctgttctgtttgcctcttggtatgttacaaactgtctccaaacttagcagcttagaacaatgaatcctttaccctctctcacaatgtttggggtcaggaatttgagcgggccttggctgatttttctgttcctcatgccatcaattgatatcacctgatgttattaagctgatggatgggctgatctggagatgcactgtccagtttggtagccactggttacctgaaatgcagccagtcctaattgagatgtgctataactataaaacacccacatgattattgaagatttggtgccaccaaaaaatttaaaatattcgttaataatttgtattctgattacatgttgagattataatatttcacatacatcagataacataaaatgtcattaaaattaatgtcacctatttctttttaatttctttaatgtgactactacaagttttcaaattatatctgtggcttgtaattgtggcttgtattgtattctttttttctgagatggagtcttactctgttgcccaggctggagtgcagtggcgagatctctgctcatcgcaagctctgcctcccaggttcaagtgattctcctgcctcagcctcctgagtagctgaaattacaggtgcccgccactatgcccagctaatttttgtatttttagtagagacggggtttccccataatggccaggctggtctcaaactcctgacctcaggtaatctgcccacctcggcctcccaaagtgctgggattacaagcatgagccaccacacctggcctgttttatattcttactggacagtgctgatctagagcaggagtcaagcagttttttctatgaaaggccacatagaaaatgttttcagctttgcaggccatgcagtctccatcatagctgttcaactcttccattgcactgcaaaagcagccatagataataatttacaatagacatagcagtgttccagtacaactattaataaaaataggtggtagccagatttggcctacaggctgtagtttgctgacccctgatctagaagatccaagattttattcatatgtctggtggcttggcagggataggtggaaggctcagctgggaccattgacccaaacagctatacagtcctctccagcatgatggtctcggggtagtgggacatcttacgtggtggctcagaactccagataaggtactcccagagagacaggtagaagctgtgaggcttcttatgaccaagctctcgaagtcccagaatatcccttgtactgtattctatggtcaaacaggtcactcaggctagcccagattcaaagagaggagatccaactctacctcttcatgggaggaggagtagccaaggatatgtgtttctttttaatctattatatcattcttcagatctcagtttaggctggtcctgttatgggctctcaaagtaccatgaacctctcttttgtagcacttgtcatagctagttttacatttctctgtatgattacttgatcactatcttgcttttctactaaactgtaggcaaccacgtgaagaggaactgtttctggttttgctcattatattcctagcaccaaacacaatgcttggttcaataaatatttgtggaagaaacgaatgaatgaatgaaccaatagcaaatgaatgaatgagtaataactgtatcaatattaatcctacatttctccatattgctgtcacgtatatcataagatactctgtcagaagccttgctaaaattcaaatatatttgattcccagtaaccttcttattttgtagttcagaaactttataaagaaggaaataagcctatcttactcttcccagtatctcaaagagggtttctgccctgagctgctcaagagggtttctgccctgagctgctgttcattctgcaaacactgctcgaatacccactgtgtgccaggtacagagagttcttctctgctgtaatctggacaggcaccagcttcccagcgtgggtttaggcttcaggtgcacactactgtgtaccgtctaagccacacctagaagagctctggggaaatatgactacttgggcagaaaaggaaggaactaagaagaggtatctttgtgtctgaggtctgaaggagcgtgtgggctcttgttcaggcaaagggcaggatgaggggaggtggggtggcagcagccagtaatggggtgggacagcggaatgcagaggatgaaacttcaggtcctggtgctctgagaagtaacgctgtgcagcatgtcacacccagaggcaaaccaaggccccagggagctgatgttgcactggagctctactctcctctcagcgagctggtgacgtgccagtccagcaggcctggcttatccaaccacaagtatgaatcggcagaaggcaatgagctgggccctgagtgctgctgggctgaggccgacctaatccttcctccacagagactgtggtgtcccctgctttgctcagggtaagaactcttgtatacctcacaagaagccaaggactacctaccaccttccacactggccctggagcctgcattgtagttatttgtggacactttttcttctctttagtgccaggtgggggaccaaggcctacatgtctttacaacccctcaatctctagaacaagtctgacactgagtagatgtagcaaatgtttgcctgaaagactacctcaataaataaccttctgaggcaccagcaaacttctcagcatttttcctgatactccggttaccactaacattctacacaaagttgtgaaataagtctttttctttgttgctctccaacatctactgtggacccctcctctcacttcctgtttcatcctctctgcactcccctgtcccaccccattactggctgctgccattccacctccctcatcctgccctttgcctgaatgagagcccacatgctccttcagacctcagatacaaagataccccttcttagttccttccttttctgcccaagtagtcatatttccccagagctcttctagatatggcttagatggtccacagtagtgtgcacctgaagcctaaatccacgctgggaagctggtgcctgtccaggttaaagtggagaagtactctctgtacctggcacacagtgggtattcgagcagtgtttgcagaatgaacagcagctcagggcagaaaccctcttgatgcaaagggatactttggggccccttcttctcccaccccagtctgtctctctgagagtcctctcgattccaggagccaccatcacacctggccctaggctgtgctgctcccgtctgtctcagaggctagataacatcagagtcctttccactggctcctgtggcagagcaaaaactggttggcatttttaaacgtgctacaccagtgtgtgaaagaaacacaggctgcatgggtttaaatctcagctgtaccatttactagctgggcagcctagggcaagtactgtgacctctctgagactccattccttcatctgtaacatggggacaaataatctcaccctgttgtgagcagtaataatatgattaatcatttagccaactcttattcatgttctctgatgggccagacatacaaagtaagtgaaagtggattacggcaggtgctcttcttggtttctggagtgaacctccatttacatggaggctcctctttttagatttctgactagttcacccaccttattcatagaccttattctgtgcttagctgacagaaatctcctctcagagaatccccccggtaaattcttaggttctttcctcttccattcccctttttgctctctccctccgaaggcaagagtttccactttacaggcccactggagaaagttatggcttctggttgtggttggaggttcattcctgagggagtggggacatttctacacttcttcacggccaatgacattggagaaactggcttcctaacccagcccacaccctcgcacacacacatcacacatcatggctagaatggagagaaattcttcatatggggcacttgtacttcatgaaagaaaatcatatcaatcttgagtattttaacatcctattacagcagggtcactgataaactaagtgtccagagtgttttctaggatggtgtgtggtctccaaattaacattagtgaagcttactggaaggattgttactcctgggccaggccaggattttgaggagagatgtgtttgctgtcaccaaatccttgacagactttggcagaagtgtgttaggcttactctggatagcttcagaggacaaaactagtattgacggaaggaaggtaaggagaagcagcttctaacccaggggaagagagagtttccaaactgagaaatcaaaaatggtactgattccttgtcagggtcagtgcttctccccactgtgtgaattacaggggccatttgtccaagattccttagagcaatactgatttcatgtaattatttgaatgaaaggtgatttgttaaatttatagtaaaatataatttgatttgtgtccctgtttgtcatgccaccccagaagaaaaattgtctttggttaggtcgaacataatggttttttggtttgcaaaccatgagcgattcccatattaggtgggagttcagattcaaagggccctcttttttttttttttttttttgtagtagccagcctaatgagtaggaagttgttctcactgtcattttatattgaatttcttttattttgagtatgaccatcttttcaaatgtatgagatagttatttccagttccacatactatctgtacatttcttttgcccgcttttagtttgggtctttggcctttttcttattgatttatagaagctcttttatacatagaaaattaatactttgtgactagttgcaaatattttcagttgctgaaatacacagtaggtgttccatgtaagagctgaacagctggttcctgattgctgtctccctcccttccagccaatagatttcagagtttgggcattacctattgagccaaagctgacaccacacaagcgcagagtatgggaacagagttctctgtctgattcctgtgagcttcctcatactaaatcaccaacagcaacctacttatcacagaatatgagaattgaacaagtgttggcaaggatgtggagaaattggagctcttgttccagttgtcgatgggaatgtaaagtgatgtcgctgctatggaaaatagtgtagcagttcctcagaaaattaaaaatagaatgaccacatgatctagcaattccccttctgggtatatacccaaaagaactgaaagcagagtcttaaagagatattcatacagccttgttcataccagcattatgcacaatagccaaaaggtggaagcaactcaaatgtccatcaaaaatgaatggataaacaaaatgtagtatgtacatacagtggaatatcatttagtcttagaaagaaaggaaattcaaacacatgctacaatgtggatggcccttgaatacattatactaagtgaaataagccagtcacaaaaagacaaatactgtatgagtttacttataccctaagcagtcaaattcatggaaacagaaggtggaatggtggttggcaagagctgagaggaggagagaaagaagagttattgtttaataggtatagaggcttagttttgcaagatgaaagagttctgaagatggatgtagtgatgactgtacaacaatgtgaatgtatttcataccactgtacactcaaaaggtgaagatggcaaattttatgtgtattatgccacaactaataaagatttctaaaacttatgagatctaatttcaccgtttcctattgctaaagatcacaaattagaaaacacgttggcaaaaggtacatgaaaataagcactcttgtgttgatcagagcataaacgtataatctcataaactaataaagatttctaaataacaaagatttctaaaacttatgagatgtaatttcaccatttcctattgctaaagatcacaaattagaaaacatgttggcaaaaggtacatgaaaataagcactcttgtgttgatcagagcataaacgtataatctcaggggagaacaatttgcaactattcttcaaccctttggtcaaacgattctgcttctaggaatatagcttactcccacctgtgtgatatggcatataatcaaggttttccattgcaacaaaagattggaaacaacgttaagtatccatcactagtggtctggaaatatatatatattattgtcatccaatagaatacaatagactaatatgcaacttttagcatgaggatactcgttacatgctgatacagaataatctccaaggtagtcatatgtgtgcaaaaccgtacatagtatgctaccatttgtgcttaaaaataaaaagaaaacagaatatgggtcaatgtttttgtttagttttgtctaaagtaactttaagtagaggcaagaaactggtaacatgtaacagtgatcacccctgttacctctgtggaagaaaactagacagctaagggacaaggctgggaggcagacttgctttccactatttatcacctttatctttcaaatttagtaccatctacatttagtaccatgatctattcaaaaatatttattaaaaaaagaaaaggtatagtctagaaggaaaaaaaacataacagacacttctagcccaatgtcctgcactgggtgctatgagagcagaggaaagaaacacatatggcttctagacaacaccgtctggggcatacatttctgctattcgatcaagaatagttgtgcatcttttcctggaaagaattgatttgtttttatcaacagacctatgaatttagtggacagacctgtgaattaattcactggttaggttttcctttttacattggctgttaaaaagctataagccaaatttatgtccccctcagtgcaaattgggcagatttctagggcaagcatttagcactggccttgtccttggctctgtatcatattcctgtatttggtttgcttttccacctgtttctcatgttggtcatctttcctgtgtatggccataccatcctgaatgtgcctgatcgcatctaatgttggtcacctctccttattctttgcttccttataagccactaagcagcctttttggtgctagttagggtaagtgcgtgggtagtgaaggagggaggagggagaggaagaaagaagatagaggttataaagcaaagcatatcctttttcttggcttcatcatgtagattaagtgaattgctctcaaagcgtggtccttaggccggcagcattgtcatcaccttatgttgttaaacataaaaattcatgggtttcatcccaacttactaagccagactttctgtggttgaggcccaggaaactctccaggtgatttttactcacattcaagtttgagaaccacaggaaaacaaaaggaaggcagatttctaagcgtaaatgcaatactaaccgattgcccccatcatgcctgttatgttggtcaagataaataatactagctactgcaataatcaatccctcaaattttattttttgccaatatcacaatccattgtagatcagttgtgggagaggtgtaaagagagctgctttattagtttattaagcaaaccagatctcttccattgtgagactttgcgattttctaggcccttggacatttcctctggatcccctgctgctaagaaggcaggagagggaggaaagagaagagactttagcagccagatctggaagaaacatcttttctgcccacaattccattggctagaagccagtctcatggcctgtataactgcaggggaggctgggaaatgtgacctatcgatggagctaagagcaaaaggaaatggctttgatgaagccctggcattgtctctgcacacccgagaacccaagtgaatcccaaactccacgtccaggtcatgttttggtgaacatcggttttcagtttccttttctaatcaagttttacctttttttttctcgactctagcactatgggactgagtaacattctctttgtgatggccttcctgctctctggtaagaacctttcagctttgttaagtcctggaatcctactgtctcctgatgagtctgaccacagcaagcccaggcctgagacttggtgggttttactcactttctactgagcattgtacaagaccacatgcaaaaaagactttcctggagaagaaggaagtgttatgattgagagcagctgatggcaggcagctgggatggagctctcccccccgtgtgcttcttcctcctctgcagtctcacatcagtgagcctagatgctcagagtagggtagcctggcccatcccatggggatgggggaaggctgctgcactgaggcccctgagacttgactcttttgttccacacatattctcttctggtcttctctgaccctgtttctgtctttctcaggctcctaggaaacaactgacagaattccaaaagtctcccttcattcggagcactggctttcacgtccctgacttccctaccctctctcactcccttccctacagcccatgcacatacctcatggttgccacggcttcctgacaactatggatgttcagctaattgtgtcagctgatttatagtggagccaatgaagctgaagcttcagagccctccatttgcacaaccctttctaaatccccctcaagaccctgtgaagggccccctagcagtgtggtcacctgtcttatgctttggtaaaatttgaataagtaagatattgtaaccacaataagttatgaccactgtctccttcctctgcaacttttccctccatgccattctcctgtctggtggtgttagcagtcaggggcattttgtatttgaattctacattctttttcttaactatccaccacctcccctcaaaattttaacagcatccagcctcacaaaactcagatcttccctgtttacagttccactttgagtttcagtttcttcatctataaacaggagttggctgcggtccctgccatgtatcctgtgactcagtgtctcgtagttactcctggcccaccccttcctgctgctccttgtctccacctgcaggcctgagagggaagccaccccactaagacagggaggtgaactgagcctgaagtttggctacagcacccacaggccaccagccatgagttcacctcctccagatggccacacaccaggcccttggccactgtccccatgtctgctgtggatgatgaggagtcagggaactacaaagagatggtccctcagatccatgctggctgggataagccttttcagatttctgtttttctgcttagcaccttgagcttgtggagtccttgagtgcaaggtctgtagatgtgccagctgatcactgacttaggtaacaacagcagcttccaacccccagggcccatgacctgctaccttagctcctggggatgtgggaggtatgtgtgtgtcagagagcaaggcaagaagactctagagaacattatccagtaagattcccttctcatcccacttcttatttatttattttatttattttattttttgagacagcatctttctctgtcacccaggctggagtacagtggcacagtcacagctcactgtggcctcgattacctgggctcaagcaattctcccacctcagcctccccaagtgctagaattatatgcatgagccatcgcacatgacttattttatttatttgataaatgcatatatacacacagtcatgaatcgtttaacaacaggggtacgttctgagaaacacattattaggcgattttgtcattgtataatcatcatagggtgtccttacacaaaactagatagcatagcctgctccatacttaggctacctggcacagcctattgctcctaggctacaagcctgcacagcatgttactgtgctgaatactgtaggtgttgtaacacaatggtatgtatttttgtatctgaacatatctaagcatagaaaagatacagtaaaaatatggtgttataatcttatgggaccaccattgtatatgactgaaatgtggctgtgcaatacatgacagtatatgcatatatatatatatcccttactttgtgcctggtactgttctaagtacctcataaatattaactcatttgagcctcacaataactctctgctttaggtcttgttgttatttcccattttaagatgtggacactaaagcccagagagatgaagtaatttacccaagatcgacagagctactaagtggcagagcttggattcacacccagcaatgtagatttagcattcgttcacttgactcttctcctaactcttgtggtaaaccatgaataagtggtaagacttcttccatggggcctgaacagctttggtggataatatagcttctgcctcatccgtgttcatccagtgcctcctccccatcacctgcagctgacacctcagttgacccaagagcttgggcccaagcccttctcatcaaagtgaccagcccagctctcaagatctgggagagaaggaagaaaaatgccctggaaacacatttccagaaaacactaaactggaacaccatttcccaccaaattttctgactccgcacactgaaagtgagaaagtaaagccgagacactctatgaaaactgagttcaggtgtcacttttgcccttgatttgccattgacacttcttagaagtttcttagctcctgagaaaagagttaccaatattgaaagcaacaacctcaaatggtaaccgtttaagttttatggtggtgagagaataagtgactatatttttggcagtacaattttaaagtggaatagaaagcccatgacatcagatcagaaaataacattgccagtaattcacacacgatgaaaagcaacaaaaaatcagattctatttgaattctttcttctcagggcacacctctgcttactgggctggtgaacagtgacctagccacagggccggcttccaaagggagaaaggagatgcaattggcccacataatccaccctcaaaatgtagagctgaataattcatttcatggcatagaaatagcaatacagtgaagcaattctgtttaacttttccctccctatattttgtgtcctctgtcatggaaatttgacacagtagtatttgctgcccctgctcttgaggataaaattggatgggagtttaagactgaaacgggcacctgtggccttgcagaattaggttacagtttgtgccttgtatttacaaagcgaaaggaattcctagtgccacctgcagaggcacttctaactttcaagctctgtttgccactgtcctggcacctccatcacacttttaggctggagccagagaggtttttgaaaaatcagtagctcccacatcaggaggaagtatctttccagtttgagttttggtagctgctctctttttgtctgagggttctctgggtcctagggctttctcatttctcttgaacaacacctctagttaatttcatgtacctggagtggtagttggaatatttcttcactttaagattttttttttttttttttgagatggagtctcactctgttgcccaggctaaagtgcaatggcatgatcttggctcacggcaacccccgcctcccaggttcaagtgattctcttgcctcagcctcccaagtagctgggattacacctaccaccacaaaatacaaaaatacacaaataatttttgtatttttggtagagacggggtttcaccatgttggccatgctagtctcgaactcctgacctcaggtgatctgcccgcctcgacctcccaaagtgctgggattacagacaggcatgagccactgcgcccggcccaccttaagatttatgtaagattggctcaaaagctcattcctgtggaaaggtccactgttttcctcccaagatttttgcagatatctgcgtgggtggttacttttgactcccatttcctgctgttgttgatagccctcattaaaaccatcacctggaggtgaatagacagtcgagacctatcattcccaaagaattgtcatggagcctaatagttctattggattcacccctttatgttaagccaccatttcagtgtttttcaaaatagatatatgttatctagtagggagtatcttacccccaaattagttgattgtttcaggagggcttttagtgggttccagagaaaatgagcaatcagacaagttgatttagtggaagacagtcactgaataggatgtgtatagggttgtttgggagcaagagtgaaattggtatggaacagagaggctcccaaggcaagcagacattttttttggaagaagcaagtgtttgagagactgtggcttatttttcctttgtgagaggggagttttaataccatttccaaaatatgtaacctggtattttgtccccagaagtactgttgagatttatggaagcaaaaaactctgtcacccaggctagaggagtgcagtggtgctatcaaagcttactgcagcctctaattcccaggctcaagagatgtttctgcctcagccacctgaatagctggcactataagtacatgccaccatgcctggctagttttttttgttgttgttttgttttgctttagagacggggtctcgctttgtgcccaggctggtcttgaactccttttaagtgattatctcttctcagcttcttaaagtcctgggattataggcatggcctatctatttttatgttttataatttcttgtactttttgatgttacttcaaatatctttttaagtatcctaaatatacttatttaaattttttttgagtaaatttatctataaattattgattttatgtcgatagacattgttctctatcattaataatgttaaaaataaataaaaaaacaaaaacaagtaaatcaattaatgcttaccacaggccagtatttgatccaacactaactcaaatattcatttctttaatcctcacaacaaacctatgaggtaggtaccattattgttcctgctttttgcaagaggaaactgagacacagggaagttaagtaatttgcctatggtaacacaggcagtgagtagttgagctgagattgaactcacgctgtccagaatccatgctattagttataatagtgtactgccctatagctttctgtttcacagctacatggcattactttgtatggatgtatcattatttgttaaaccatttaacttatttccagtgtattgttcttataaacaatgaatacctgtgtacctctaattttgtgcacatgtatctttttgtagaatgaattcttaagaaattgagttgctaagtcaatgcttaagcccataattaattttcttacatattaccaactgtcctccaaaaaggttgtaccaatttagaattttaccagcagtaaattcagcagttaggacccattttcctaacactctcgcggacactgggtattaccagtattttttttaatacgtgccaatcaaatgggcaaaaagaatggtttctcactgaggtttaaattgcatttccctagttattcttgagatttttcctttcctttcttcaacaattacttattgagtgcttcatatttgtaagggacaattgcaggtactggaaatgtcacagtgagaaaagtgacaaagcccctgctgtcatggagcttattctaatgggagatgtcaggtgctcagctgagctgggagagagagagctgagttgtcaggtgtcagaggagccaattatagcagcaaaacaaaaataaaatagttcagcttttaatctcttactacgacggtataatcaagaggctaaaatgggaggaagggcagactctgcctgttccatttccccacatagagtgagtataccagtcgagggtcaggtaatcagtgcagacttagggggtcgccttaccattgaagaagccccaaatgaaaggctctagcagttttatggacctgggggtggaggaatccaagggtggggagaattcatgaggaaaatgaggtgagagggctaggagtggaaaagtacaaagtactgagttagcgtggggaatagtgtctttagggctaggagtggaaaaaatactaggtactgagtcagagtggaaaacagtgtcttcaaggcagggagtggaaaagtgctaggtactgagtccgagtggagaaaagtgtcttctctatgatgaggaggcttcagcagaggtgcctgaagacctcaccccagagcctcagataaagagacctaagaatgagggtgcctgggctaagattgcaagtatgtgaaaaagcatgactggcgggaggctgagatcttgattgcagcccccttcagagactgccatgcactgactgtgcaccaagtctgctgtagaaagggcaacttcctcagcaaggcttgtcagattaagcctctttaattgcctgtggtcaggtctgaaaaatcacacatagatttttaatcagaacccagacatctcaggagagacagacaataaccaaacataccgtgtcatgtcatgtcatgataagtaccacaataaatataagtcagcatgagggacagaatgcccaggatgctatcttcaatagaatggttagagaaatctccctgggaggtagcatttaatgaaagacctacatgaagtgaaggagaagctatgagactgtctggaggaagaaccttctggacagagggaacaacatgagaagaggacttgagacagagtgtgtgatcttttggaggaatgtcaagggaggcagtgtggctggggagagtaagcaggggaaagaggcctgataggtactggggacccaattacatgaggtcttgtaaggccaggggaaggactttggatgtagttctcagtgtgaggggaagggatctggatatatttttcagtttggtggaaggcatcagaggcttctgaacaggaggattatgtgattggagctgtatttttaagggatcattttggcttgagaaactagacccggggacaaggacggagcaggcagatgagttaggagacaattacattagtctcctctacccttttcttaacatattggagttcagctctggctgtagtagttctagatctcctcagacacacttgtgtagagcctctgttgggtattttgggtacacaaatgattcatcttggttatacagatgatttagatgattgtagacagaagagggttgtctggtcattcccagacaggggagcattccttgagatagagtagaggaaggctgaaggggaggaagacagtacctgttgctatctagatagagacatccagcaggaagttgaatacaggtatctgaaactctagtgaaagttataggctggcaataagcacctgggagttattagcttttacttgacagttgaatccgtggggctagaggagaaaaaccaggaaagtatggagaataagaagaccaagaacatgcactcaaggttaccaaaattaaagagtgatttgagaaaattaacaaggaaatcagagattgggaaagaatagagcatttcaatgaggagagatgccaacacttgcatttgacacagcggtcaaatgagttgagatctgaaaagagctcaagccttggccatggtgtgaagtcaccaacaacctttgtcagggagtttcagtagagaggtgggggtgggaggctgggaataaaggcagcaattgctgcttactctttcagggagtttgactccaagggaaagagaaactaaaagcagtagcacaaggtttgtgtttgaagtaatggaggtgaaccaggtgaatagcctggaggccgagtgaagtgagacaggacactgcagatttggaatgtcaccagtccgcacaactgaataatttcctccagaactgctcaattgcccagttgtaagaacagatatgtagaccaaaagtagagtgtccccagggtaaattttatagagacaaaggggtgtgtttattgaagttgtggaaaggaataattacaaagacatactattgttgcattgtccaatataataaccactagccatatgtgactacttaaatttcaattaattaaaattaaataagattaaaaattcatcttctcagtcatactagctatgtatcaattgctcaatagccacaggggctggtggctatcatattgttcagcacagagacagagcatttccattatcactaagagttcttgtggaaaacactgcactacagggtctggataaagctgaggtcttgattaagttgaacaacagttgtagaaggagtaagcaagagcaaaacctggatgaataggaggttgtggacggagattagtatattgagattaagattctagggactgagctgctccaggtgaaaagtttcagggttatgtcataagaaggtggggggcagctgctgaaatagtctgcgggtgtagacctgtggagttgacaagatcaaagaaatttgaggcaaggttgttagactcattcatgaagaagtcacccaaattgttagcaagaccttgcatctaatgccaaaatcctcatttagcaaggtggtagtgacttagtagctacaagcaatgagaaagtcagacacctcaaaaggggaaggtgttgctcaaagtccccacaaagtgtgataaaacaaacagtagctggggctggagcaagtggcttcctttgggtgaagccagatttcactgaaataataacctcagggaaacagtcaatgaaggggttaaagatgtgggagagtttccttgtagtaagtaatggaatgaggctttcaaagggccaagtaaaactttggaggaagtttagtaaaagaaggaattttttttagtacagataagcataggaacataaagaagagataattcttaaacatataagatatgcatttggggatagcagccagggaacactgaagtcccagtggggtcagagacttcataaggctagcaaattacagtttttgagtggcattccaacagtagagtgtattgctcaggaagtccttaattatcctttgaaacaaattccttcagctgattacgaaggcatctagctggattcttgagcgacttgttcctgacatcatagcaacccattgtaactagacttcgaccattcctcttacccaagtgctggggaagggagagattctcaatgcttacccacctatggaatcccagtaagtccagttgctaggtggcttgaggtctggggtcataaaatggaaggcctgaagtcatttggtgatcacagaccttgagccaaactttccccatttagtcagagaaaggattagcagcatcccccatgcctggctctgtgtgagatcatggaagccagtggttggtgaggtgctatggagtataaattgcaaaatactttcagttccactcagaatggatttcaaagtgatttccaccccatggggaggagagggagtctgaggagggatggatggaaaaaaaattttcatgtcattttctgtgatccactctggagacagaggcagagattctctacaacagctgctcaaactatagctcttgttaaaatggaggttctgaatcagtaagtcttgggtggggccagagattccgtgtttcagaccagcccacatgtgacgtgaatctcattggtccatacatcacactttcagttgctaggtgaagaagggagcactcgatgagtggaagagaaagccgttgtaatctttgggagaaggggcctgggtcagcggagttagactggtctgtgagtggacagaatggatgggaaggaaagaagatactgtgaggctctacagaaaaaaaaaaaaaaaaaaatatatatatatatatatatatatatatgtaaatcaagaagacagaagcagctaaagacgaagtcatttccaggtccagaaggcacaactgacagctgagtaataacataacattgactgttaattggcagaatttttaactgtgtgtttggtttctccatcaggtcatctgtcctatattacatgacaatttagactaaaaccagtatttcctcagagacaatgctagaagcttttacagtagggggcactcttgcattacattaagagctcagcaaagaagatgcagaagcctcaggtttgccttgtaaggtgattcataaacacactaaatcttccttaggtctccctttcactgtcagggtacgcatatagattttccttcctccctccaataccggtacgcatcctctacaggtggtgcattttatacctcaagtacttcacagggtcctagtgagtgtagtgaaataggcagtgattcatatttgtgcaaactcccactgatgcctgctgtctgcttccctaagagttcaagaccaccaccaaccccttgattatgtgttctcactgggccactctgtacacagtttagtttgacaagtgcatgtcactgttatctgtccttctattccctctttcaagagaaaccacatcaatttaattactcccccacttagaactcttcaaatgaagctcctctcatctctctcatcaacccatctcctccctttcctcctcaatgtcaacatgccttcacataaatcctgaatgatgaaattttatttagaacttacactaacttcctctccaaggtggcatctaacttcatattaagtaagaaacagccttcccactctccacccccgcacttctcacccaccactgcttactttttttttttttttttttttttttttgccaagtctcaagtaattctgtaacctagaaaaggtcctacacaaaccccgtgatcattcacatttaagtagttgggtggcccacatccttcccacaaaccccaaagtgtcctcaaggactaaagcctttctctcaacccttccagcatgatgtctatggttgtaaaattgtccagggtcagtgcatactgggagcagcaagtttgtggtgcctggggtttccccaatactcccaaagcacatcctcacctgcccatctatgattcattttcagcatttcactcatgtgccttaaatggtcattgaccaccacaatccgaaaacagccatcaaatttgcccagttctctttctgatctctgaaagagcttagagaggtcactgaaaataaaggccttggttcactatcgaagtcatttctaaagcatttgacatccttggaagtgctggccatgggagcagcagtcataggggaagttctgtaaagggagctatttgaatttcaaagatgttactcaacgtgattccccaactaatgaagtataataaaggggggctataatttattaccattatcagcaatcttttcaccatagcagaccaaggaatatgtggatgggaggggaggggaaagcttttggtgatggtgtagaagttatggaacctgtaacagctacagtgatgaaaactaaaattaaggttataggaaggtaactggtgggtgaatgggttgtctaactctactggtttttccctgtcttgcaatttaaattcacagaaccacagtactagaaagacccttggaacatttagtcaaccacttcattaatcagatgaggaaactgaggctcataaagattgcagtttgtacaaggccacacatttagtcagcggtgaagcaaggacaaaggtcctaatctccagatgccaagcagatgtgcacagttccagagcttaatatcttattcttcagcatgattactgataagatagtatctgggtattgtataaagagaaatggaggttttttcccctttcctcttgtttctccctccctaatccttaaccttcttttttaggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttggtatgtggtcaatggtgtgtgttcagattcttagccttctcagatgagactgcaaatgagttagaaaaacactggagggggacttgaggggcccaggggaaaaggggggtctatagagagaaggcagaggacagccacttctgggaagtgcatttgaagggagtgtagagtctgggagtagggaactgaaagtcttttgtactttttatagtctgcttctgaaggatcagtaaaaatctgctttggggaaaaaatagagctaattgaacaaagataatatggctaattacctatagtaaaaaccatggataatttggccatcacaaagtttatataaccataaaggcctcagatgtcttacattcattttttccttgggtccaagatttttcacctactaaatctttgcctggagctcctagcaaagcggacagctgacacatttgggttttcccttcagcctcctctaggttgcttatgagttgtttgctgccacaaccatgagcctggtagacagaagggaaaaaaacccaacaaacataacccacaaacttacaaaccagctcctctgcttcacgagaccttggaaggcctaaatgccactacagatttttttaaaactatcacacagtaaaattatttttttttgttttgatatactgttctactgattgtatagatcttgtatagatttaggtaaccgccacaggacatagagcatttctatcaccctaaaaatttccctcaggctgtcccttcatagagtcataccctgtctgcactcataacccttgttgggcatcctatagttttgtctttttgacagtgtcacataagtgaagccacacagtatgtaaccttttaagcctggcttctttcgtttagcgcgccttcgagattcacccaagttgttgcacatatcgagcttgtccctttttattgctgagtagcattttattgtttatccattcaactcagtaaaagacattgggttgtttctggtttggggctcttatgaataaggctgctgtaaacgttcatgtacaggtttttgtgtgaacataagttctcagttctctagaggaaatacccaggtgtggtattactggatccaggttaatttttgatgaaacttgaaaaggcagatcaacacctattctaaaaccatagagtaaaacagaagcaaaagtaaaaatagaatggagagctgctccctttgaaccctgtgtgatttaaactaggctgcagggctttaggaatagttaaccaagtgctaaatccgtgttttcaaaatgtggtcaggtaccattggaaatgttttaggtgggacacagataagcattttgaaaagccatgttgtatttgttttaatgtatattagaaaaactctaacttacgcaacatgtgatttcacagatcttgttaatgaagctaaacacggtctggcaattcaccttctacaggccacatagactccaagaagactgctcaaatagtacactgatatagcaaaacttataaagatgacatgcaaatgacagaccttttagtaagaatacactaaattataaattagtttgtagaacctgcaaactacctagtaactataaaagaacaagggattttttctgacagaaggcacatgacacaggtctagggactccatgccagtgatcctgaacagccagaaaagtgagaatggcaaaggcaagagaaacactgtgtttattaagatcatgtatttttccctaaaatagctggatttggccttcttcttagagtatgttatgaagacactttgatgctcatgccaaaaatcagtgttctgaatttcgaattccaaaatatccacccactcacttaccacaatcctgcttgggtttctgaaagatatgacgcagggcatctcagcaccatgaactctgtcagttcctggtgagactccagctcaattccttcctgctctcttagtctggggagctggaatgtgccccatgggacacctgggccctagagtcagaccacttctccttccaaagactctactccctggaaacagtggcttcattgtaaatctttggtgactcaattacagccctcctgtcacttagagagcacccctttgatttggataagcaggaagtaagcatggctgcaaactctattgttgaaaaataaacatgaagtcattatgtggcactcaccttgggctgagggtcacattttagacaccctgaggctcccaggtgtgccccaatgagccccagatcaagtacccagttatttgctattccctcctagatacatctaaacttagattgatttttttttatctctcttctgctttcagctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctataggtaaagctgttttccaagactatttctttcagcaggtattatacacaaatgcttaaggcagatcatccaatgtccccgacttgctaggaaacctccaactgggccattttatgacgctgttaggaaggacccagatggaggtctcctgcttctcctgagtgatgcagggtccaggaggctacgagcctatgttgcacttgaagaaatatgcttttagccctgaaactgactcagtctcttggtttacctttggatggaggattctgaagttttgatttaaaaatacaggattcctccaggctagaattctttctttgattacaacacatacatgcgcttgcacacacacacacacacacacacacacacaccatgcatacatgcagacatacaaatgatatttattgtgagtatagaaccatttgggacattattggtcacaggagtgaaaacaaaaagatatgacaccccctctgcccttgaggaccttccaatagaatcagaaccctgtaatgtgcacacatgaaaaactggatttttaaaaggttgaattggaatctaaattttattccatggaaatatctgactaaatttaaaataaaagtgactggtaatgagatttatgggcattcagaggtaggcaagatccctgagggtcagggaatggttcctaaaggaaggggtaccttgtaacatgtaaaataaattattggggttaataaatgtggtgaggaggggagggcattctggatgacaggttcccaaaactgtggtgacttccgtagctgaaaaaatttgagacagtatctgggctaagcaggtgagaggaccacagtggatcagctgtatctgacgtaagtgcaggaggtatgtcaaagaaagccttggaggcagaaatgcttgtgtgttcacaagtattcttcagggacaagttcagtggaggaaaggattgaaactaagcagtagccactaataggagcctgacattttaaagtcctggctttacccaggagggcatgtgtctatatttgactcctcttttaagaagctgtaactgcaagattccctcctggaataaaggtggtctgcatctaccctgtcccatcactgcctgtgctgaccttgacacccacatctgccttcttcttaccttgaccccttctccagcggtgatttcttggcttgccccctccagtgacatccatccaactccttgctccataccctggctttgtcacctcctttctcccagtgtcttgttgttcagatataacttggtctgtgaacagcccacggggccagtccccatgaaccaactttacaactgggccaatctcatctcctgctactgacttcttcctattcagacacttcagcctctgagaatccagtaaatggtggagccaactcgtcctgtcccagttgcttctcctgtatcctctcttggccagatagaagcctctccaagctatgcctgaagttcagtacctccttcaatgtgtaattagtttgattggtggccacaagatggccatatatgacatgccccagggccctctgttacggctcccatagtctacaaattaacaggggcttgccaccactataacctcatcatggctcaccttcctgctgcttctcaactactgttctgccaaacttcaacaggtacccccatcttcagaaatgtttcagctctagctgcctcaggaagatggggcttgcctctctgggtttcccttctatcgcttgatcagagataggttagaccctgagtcaaggggccttttttgcatgttaaaaggtagcagcctccacgttagtaagtataacccctaaccccctttactgggagtgccaaactggctcaagtggaatagactgggacagactcaaaagggattaaatatggcctgcaatgccaacaacttcttaacatcccagaaacagggcatgtgtctacaaattatagctaagctaatagatcagctggtcctaattttcctgaaatttgggattagctaccagaactgttcccaaaaatgtctttaaagtgggcgactccgttctaagttttccccacaaagcctgttttccaactccccagaaacttaggagttctcatgtaaggaagtagttcctgaaggcgtgaaggttcctcaaggcatgaagaaacatcaaaggtttttcagtagatgagatatgctgaaagccatgcagaggaaacctgctgtgacctcagtaggaaaaaactaaacaaacaagcaaatgaaaactagaggtaggggcctgtggaagctgttccatttgtccaagtgagaggtgtctggagattatagtggacagaagaatcatcacgagaggaacttcagggcctgggaactgactgcagaggggggcaggatagcaggcacggcacaaatgactgcacgtgcagagcctcagcacagacacctcacccagattccagaatcacgggccaggctgaccctcttcttcctgatcatggtcggtgttatccccacctccatgaaggcatggcagctcagtccaggcatttggccagaggcatgggctcgattcttaggtcgctgctgaggccctgagcctgggactttctatggcctcctattgtggatttcaggcttctctggccttagagccctggggagaggctggcaggtaaataaagagaagagcagctagcagaaaccttttgtaaatgactctcctggctgattgaaaatttgtggtcatttgtagagcttgaggaccctcagcctcccccagaccacattccttggattacagctgtacttccaacagttattatatgtgtgatggttttctgtctaattctatggaaatggaagaagaagaagcggcctcgcaactcttataaatgtggtgagtgagtccttgtcctccccacagactgtcactttgcacctacttcccaatcggctggctgccttccggagcttgttggctgagcctagactggcaaaaagtcaggaagttgttgggaaaaaaggttttcccttggagttttgagcctatacagactggcagtagcagataatgctgctcttggacttcaaagaaaggcgacatttctaacctctggtttacaaatgtacttctggtttccagggaaaactgattattacttgctttatctacctcacttcatgaggttactgtgacatatacataaagtaaaatggtgaaaccactcctaaatgttaaagattgtggacctggtggtgtttaagcagggatatttgctaaatgaccacaagaatcagcttctcgtctctaaaaaaatctaggtttcttatgaaataagttagatgaattattgcccattgacttataacaaacaatattaactttaactaatttctaagtaatacatatccattatcatatataccaaaaataaaataatctataactccactaataagaaaaaatgattacacaaatatttttggtgcctatctttaagatttttctgtgtatcaatctatgttgttttccataattaggattatcataagggttatttttcacaatttggataatatatgtactgtgttctaattttgttatactaaatgtagcaagacaattttcaatgtcataaatatcattctacagcatcatttttaatggctgcaagatattcccttttgtggatacaccataatttatttatttaaccaacctcattttttggacacttgagttagtccaatagttttgttattataaacaccctccccactgacttctgttataaaaatgtttcatggggacaaagtggtccctaactttataataatgccatgcctttttgtagtttggtctggttctaagctaagattggactttatctcagtaattgcctccagtagtaattagtttgattggtgctaataattaaggtaaccttctaactcacttatggtagaaagcacaagatgagtattgcctctggccagcatcttgtttttcagtatactgattttaaaatctaactagaaaatagatggatgacattagcagtcattcaatgcatcctgctgtactttaaaaataagaaattggggagcaacgatcgaatttaaataaattaacacaaagcatgtggcagagccattcaaactgccaatgtatggagtgtgctgcgagatttctatgatataaaagtataaaattcctagcacagatgtaaagacatatcatgcttgtccaggctttgacttttcaaggtgagagttttgagcttcactttctttcaacctcattgccatttaaaattagtcaaatatgaagaagtgacttacatcttgggaataagctgtttgctagatttttcttcacattagaatgatcagcttacaaatgaaacaaagaagggttggagaaaaagattaaggatgtttcttcctccatgaggcaatcagaaaaaaatcaggagactagataggggagataaagaggatatgtgtgttcacatgagagaagttagaaggtggttaaataagctctgtaggtacagatgagatggtcagattgggctgagtggcacatacatgacccctaagaatgtaatgaagaatattggtaagaaaaagttatttattcagacagtcatccatgccactgagtttgatcaaagagagaagccttgctatcactgtagggagggaggtgcaacaggtataactatgccattatagatatgatatatttgtaaatttggattctgtaacttcagcaatatctgccattgctttgtgggtactcctggcattggctatgtgataggtaaaataatgccccccacaagacgtccacctcctatactccagaacctgtaatatgttatcttacatggcaaaaggaacttcacataggtgattaaggcaccaagcttgagatggtgagattaacctggattatccaggtgggcccaatgtaatcacatgagtcagagaacctttcctagctgggatggagaaatgaactggaagaaggagagatctgaaacttgagaagctcaacccagcatttctagctttgaagatggaaggaggaagccatgagccaaggaatgtaagtagcttctagaagctggaagtggctctcagttgacagccagccattaaggaaattaggatctcagttctgcaactataaggagctgaattctgccaagagaccaatgtggaaacagcagatccctccacagagacacaagcttactgataactggtaggaatttctccaaaagtggagcttcctcctactccagtgttaatccctttctcagaggagacggtcctcaaactaactaacttggcaccaaaagtcctatccagtgttttctcattatagtttttctatgcctcaactgtatatatttacccagtttaggctgtttaaatgaataaaaaggaaatgccatagttattctagccagtttccaatctctcttctctttttttgttttgtcaaatagggcagataaggcatgagaatttataactatgaattactgtcttttcccaaacagaaatcaccctatcagcttacccattgggagaaaaactaaaatagctccccctgaaattttacttcctcatttgggtcttgtgtgactgaaatctgtatacaatgccctagcaacaacggtttttacagcttgcctccctagaacaaacctaggagtctcagctgtttcaggaatgatttcttaaaggtaaagtgcctttttcaaaagaaattattattatttttttttaattttttttttgtgtgtgtgtgagacagagcctcactctgtcaccaggctggagtgcagtggcacgatctcagcacactgcaacctctgcctcccaggttcaagcgattctcctgcctcagcctcccaagtagctgggactacaggcacgtgccaccaagcccaggtaatttttgtattttcagtagagatgggttttcaccatgttggccaggatggtctcgatctcttgacctcgtgatccgtttttaaccaacatttaaacagaaatattcacaggcttaaagactgaaagttagtgatatcatcacatttccccttcaaaatgctgaatttgtaagcaaatttaaaagtttagaatctaccttttaattgtctgctttcatttttttgacagtggctttttttgatatggtgactattttgtcatgggtataaaaggataattcattttgtgttaatctgaagacatctgaaatactgtattcaactataagtacctttttttacatttataagattctttttcaaaatttttatttgaatagttttttgggaactactgaactaaactaggtggtttttggttacatggataagttatttagtggtgatttctgagactttggtgccacctgtcactcgagcagtgtacactgcaccagtgtgtagtcttttatctctcacccctcccactctttcctctgagtccccaaagtccattatattattcttatgtctttgcatcctcatagtttagctcccacttatcagtgaaaacatacaatatttgtttctccattcttgagttacttcacttagaataatggtctctggttccatcaaagttgctgcaaatgccattattttgtttctttttatggctgagtaatattccatgagggatatttaccacattttccttatccactcatgggttgatggacatttaggttggttccttatttttggaattgcaaattgtgctgctataaacatgcgtgtgcatgtgtctttttcatataatgaattattttcctttgggtatatacccagtagtaggattgctgaattaaatagtagagttctacttttagttctttaaggaatctccatactgttttccatagtgtttgtactagtttacattcccaccagcagtgtaaacatgttcccttttcaccacatccatgccaacatctattattttttgattttttaataatggccattcttgcaggagtaaggtggtatctcatggtggttttaatttgcatttccctgatagttagtgatattgaactttttttcatgtttgttggccatttgtatattttcttttcagaattgtctattcatgtccttataaacaccattatttttaagaagaaactttacaaaaatagaacataaccagatttataaagcatctgggaactcagtcaattaagaaatagctcaagtaactgatgatgcttcacctgaaagaaggcctggagagaacagagatactgtcttcaaatatctgaagagctaccatgggatgcaaagattgagcttgatggtatgactctgaagggcatctctatgaatgaaggttatgagagggtataaggaattaagagagacttttctaacaattaaaaggtcttttaggccaggggtggtggctcacacctgtaatcccagcacttttggaggctgaggcaggcagatcaccttagatcaggagttcgagacccgcctggccaacatggtgaaaccccatttctactaaacatacaaaaattagctgggtgtggtggcaggcacctgtaatcccagctacttgggaggctgagagaggagaatcgcttgaacctgggaggcagaggttgcagtgagccaagatcacaccactgcactccagcctgggtgacagaagatcaagattccgtcttaaaaaatataaataaataaataaataaataaatagtctttaaaattgtatagaagaagtagacttctgcttcctccaacaaaggattaactgctataggaattgccctctttccataaacaactagaaagcagacaaaatatatgaaacaactgttttcagagatcggatgacagacagcagaaaactgtagtccctgagtgaaggaaagaaaaaatgagataagccctatgattgctctagtttgctgcctggagccagtgtccaggcccctctgaaggcaggggagccctgatactgaactaggaaaagacattgcaagaaaagaaaactacaaacatctctcgtgaaatgcttaacaaaattagcaactaaaatctagcaatatgttaaaagtataatacatcatgatcaagtggggtttattcaagaaacacaggtaagctcaacattcaaaaatcaggcaataacctttactacataaataaactaaaaagaaaaaaacatatgatcatgtcaatggatacaggaaaaacttttgacaaaattaatacccattcatagttttaaatggaaagaaaagctctcataaaaataggaatacaagatgacttcctcaacctgacaaaggacatctaccaaaaattcttctgttagcataatatttcatgatagaagactgattgcttttaccttaagatggcgaatgtggggaggatgtctactctctctacttttgttccacattgtactggaggtcatagccagagaaacaagactagaaaaagaaataaaagacatacagattggaaaggaagtaaaactgtcttttttcacagataatgatcatgcttgtagaaaatcctgaggaatctatcaaaaacctattaaaactgataagtgagtgtagcaaagacacaggatacaaagtcaatacacaaaatcaattatttctatatactaacaaaagcaattgtacattgaaaaaaattaatagcatttataatagcatcaaataatattaaaaacttggaaataaatttaacaaaacaagtacaaggtctatatactgaaaactatacaatattactactggagaaattaaagtaaaccaaaataaatggagacataggccatgtttatgaatcagaagactagatgttaagataaccattctctccaagttgatctatggattaaatgtaatcacaatcaaaatcctggtaagctctctaatagatactaaaaatcttactcgaaaagttatagggaaatgcaaagaatctacaattgccaaaacaattctgaaaaataagaacaaaggttaaaaatacaaaattagccaggcatggtggcgcatgcctgtaatcccagctactctggaggctgaggcaggagaattgcttgaacccgggaggcagaggttgctgtgagctgagatcgtgccattgcactccagcctgggcaacaagagtgaaactccctctcaaaaaaaaaaaaaaaaaaaaaaaaaaagaacaaaggtggacttaacctacctaatttcaatatttactatatatagtaattaatacagtgtgatattggtaaaaggacagacatatcagtcaatggaacaaaatagagagtcaaaaatagattcacactgttgacaaagctaccaaggtaattccatgcagaaaggatagtattttcaacaaatagtgttgggacaattagatatccacatggaaaaagtatgaacctagacacacacaaagtaacttatatattaagaattaaaatgaaaggacttccaaaagaaaacagaggagaaaatctttgtaaccttaagttaggcaagtcttcttagataggacacagaaagcaaaaaccatatcataaaaagataaaatggatgtcatcaatatggaaaacttttgttctttgactttgtttaaaaaacgaaaagtcaaaccacagacagggagaaaacgtttgcaaaatatatatctgataaggacttgtatccagtatataattacatattgctactcattagtaagaagacaatccatttaataaaaggcaagaagaagagacttgaacagatacataacagaagaagatatacagatggccgatgagcacagtcacaacatcattagtcatcagggaagtacaaattaaaacgataatgagataccactgcacaccctctagaatggctaaaattaaaaggtctgataaacatcaagtgttggagaggatatgaagcaactgaaactctcatatactgctatacaacccagaaatcctagacatttaccaaacagaaattttaaaaaatttaaaaatatataaagactcatacacaaatgttcatagcagcttgcttcataataccaaacctggcattctaaattttcatcagttggcggtggtatatttatacaatgaaatactgcaaagctatagaaaggaatggactactaataatacacaagaacatagataaatttcaaaagcattatgctaagtgaaacaatccaggcacaagaagaatacacattatacaatttcatgtatatgaaatttgagaaaaagcaaaactattttaagtagattcatggttatccatgggatgggggaaaggaatcagctgaaaagcgaactattttggcttataaaaatgttctcgatcttgattgtggtggtggttacgtgactatatatattcgttaaaatcaccaaactctaaactgaaaatgattgggttttattatttattaattatacctccataaagctgattgtttttatcttttatttttattttatttcaatagtttttggggaacagatggttttcggttacatggatgagttctttagtggtgatttctgagattttgatgcacctgtcacccgagcaatgtccactgtacccaatgtgtagtcttttatccttcatccacctctctctcactcttccccccaagtacccaagtccattatatcattcttatgactttgtggcctcataaaagctgattgtttttaaatacacacatacacacataaaagagaacttccagtgacaggaagtgttcaagaatgctctatttagtaaagacagaatcacaaaaccatcagaggtattgttgagtggattcttgtggtctataaatacctccatggacacccaggttagcaacctgttggagtttacgtgggacaatagcatcatcacaacagtcagcctagagaaatttacatcccaagttgtgtcagtagcaagtccctatcaatagcaactcaggctttgtgaggtctagctggctagaaatttcccacttggccttgcccatgcaacattgtgtaatattcttagcaccatctggctagccgatttaggcatcaacatcttcaagacttcttctcctcctccttataaaccttgctttcagaaaaggattagaaactcttccaatcacaaaatgattgctaaaactaaatatattacccctcccaatggtattttttggttagccaggatagagatataagtgaaaaatctatttccagtgttagaatttaaggcacagtgagaaagggaaggcatatactttttgaatgcaagaaacttcttcccaatccccctgaaattgcatcatttgagtaactatctcttccatatataaagtcacacaatttctctctcagtcccagaactttgaagccttttcaaactttccttcttttggtatctaggaggaatacatttttgaagattgttcttggtgtctttcaggaaccaacacaatggagagggaagagagtgaacagaccaagaaaaggtaaatcctgaccctgagacattgatgagagagaggtataatccccagagtgcctgttacttgaataggcttatgcctaacatatgttgagacctcagcaaacctgaactaatggagagggagaggaaaataaaactagttaagaactggaagaaaataacctgataatggatgacagggtatccaatgcacaatgcccagaaagcatgacaagctctgtcatggtcaagtaaaagtcaataccaaagacttcagaggtggtgaacatgggcttcatcttatctgccacagtaaccccagtacctggcacagtgcctagattagtgggcatcctacatgtgtggaatgaataaatgaagaagtggggaatgataacatgtttgcttcagcctgagcatcttagtatttgctatggccctgtttagatgttcttctgccacttctttacctcattcttcagatcttgcctcaagcagcactttcttaaaaaccctttcccaaactagaaaatgtcaacttgttacagtgtcatgtggatcccttggctttttcttaataacaccagattatgcttacatatttgtgtaattatcttattaaactctataaactagacttaactaaatcctatgaagagcagagaccataccagttaagctcatcattgtgctgctagcacttagcatggtgcctggcatatagcaggttctcaataaatgttgaaagaatgattgatgcatgatgaatacataaaagttcgtggtgatcagtcctttcacaacgtgaagctatcagatagtctgtacctctatccctcctgagaaattaagctctcaggaatatcaaggctctgactgcatacccataggatcaaagcaaccctcagtcacaagcctggtttcagagatagggtcataacccccagggtgcagagacaaccgagagtacccagcactaatccagatataccagccactgtgattctagcaacaaaactaataattccgggcacccttggacaatgagaaagggtgctgaaatcctgcctaccctgtcacactcagtttcagaaatggtctggaagagcctgcagagggcaggcagcagagaaccggcagagggcatgggaagggccaggcagaaataaagggtagctcttgaagcatagatgacagtgtagaccgtggttcttttctcttgctttctccacctttctcttcaatagtttgtttctcctcattgctgttccaatggcaacctctattctgccctatcattgaaatctagaaaaagaaagtagctcaaatgtgaaatatcacctaatcttttcttctatttctccagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaat

SEQ ID NO: 3 - DNA, 23192 bp, Homo sapiens
ttagataggcagaaatttaaaaagatctggctgggcacgtggctcacacctgtaatcccagtaccttgggaggccaaggtaggaggatctcttgagcccaggaatttgagaccagcctgagcaacatagtgagaccctatctttaaagaaaaaaatctgatcatgctaagacctgctgaggggagtgtaaatgggcatgtgcattttggataataagacggcaatatttaacaatgcagtgtaattactgagctagagtgttggaagactttcagctcccctgcaacattgtttataatcaggaaaaactgaaaagaagcataaatggctaggtatgagatctggcagaggacacatagtgggtctcaaaagaccatcctggctaacacggtgaaaccccgtctaaaaatacacacacaaaaaaattagtcgggcgtggtggcgggctcctgtagtcccagctactcgggaggctgaggcaggagaatggcgtgaacccgggaggcggagcttgcagtaagctcagatcacggccactgcactccagcctgggagacagatcgagactccgtctcaaaaaaaaaaaaaaaaaaaaaaaaagagggtctcgaaaatgttagtactgttttatttctcaagaataaattgtatacagatgtgttcaattccatattttctatacttattttgtatgcttaacattttcacaattaaaaaattaatttggtgaggctgctggagaaaaggtactcacacaagctggcgggactgtcaattgatataactacttccaagagcagattagaactggtggtatagtgatgccactctttttaacctcttggatggacaaagatagaaaggttggataacagtttgtgttggcaaacaggcactctctttgcagatgggaatatagattgaagacacctccttgcaggtaattttttggcaatatttgacaaaattggaaactccccttcacctagcacaatttccttgaggtatttattctaagaaaataagcaattttagagcaaagatttatctacactgaagtttcccatagcaatcacagtattgtttctaatattagtaatacaaaaagaaacaacctgtatgtctaacactaatcgattctaatttatggtgcaactgaacaatggaccaaaatgatgctgttggaagtttttaatgatgtggaaccgcttgcaaattattaagctaaaagaaagtaggttacaagatagcaggaagaataaaccattaaaaataccaatctgtgcactgacaaatgttataaatattttacgttatgttatgttataaacattttataatataaaaaaatgttaactgaagttacttcctggatgaattacaggtgatttcattgtcttctagaattttcttttccaaaaatgttgtgtatgcgtgtaattattattttaataggagacactctcctttggtgatataatttaaacaggacggtactgactgataacctcccggggaaggcagggagccaagtactacagacttgtatgtttccatggaaatctaacgcgcctttgattatcacagattctggagaagagtgaggacttgggttcaccagtgcgttcccaaggacaggctgggcttctgaggaagttgcccaccctctcggaatctggtttggcctccgtaaaatgggcagatcccgctcggatggcccggttcccggcttccttttgcgggtcaacggcagcgtcacgcgcgcgagcgcggtctgcaaagcccccagcgctgggcgtcacgcggggattgctgtcgccgctgccagccgcagcagcgacgcgaactcggggcgcccggcccgggcgcgcgggggcggggacgcgcacgcggcgagggcggcgggtgcgacgggggcggggacgggggcggggacgggggcgaagggggcggggacgggggcgccccggcctaagcgggactaggagggcgcgccacccgcttccgctgcccgccggggaatcccccgggctggcgcgcagggaagttcccgaacgcgcgggcataaaagggcagccggcgcccgcgcgccacagctctgcagctcgtggcagcggcgcagcgctccagccatgtcgcgcggcctccagcttctgctcctgagctgcggtagggctcgcgagcgcctgtctcgcctgtcgccccccgcccctccacgacaccccctcccgtcggtcgcttgctcacgacgcgctctctctttcttgtagcctacagcctggctcccgcgacgccggaggtgaaggtggcttgctccgaagatgtggacttgccctgcaccgccccctgggatccgcaggttccctacacggtctcctgggtcaaggtaggtgctgcgatacccacgggctggggtttggtgggctcatttgaagacagcaggaaccatctcccctaggctggcgaccctctgtggctgccaggtgggggcgaggggcgtctcccgcagctgaacttggagtacccagcctcccgtcgcgcctcccccaccccatccgcatccaggtacagggccgaattaggttttgctctccgcagacctcaatccccttcctgtcactgaaggtggcctgagatgaatgatccacttaagatgttttggaagggcagagactctcatttggattaattctggaggccacctgtggttgtgggccagcaggtcaggaagaaagcaacagggacctagatttgggcattggacagggggaatgtctccagacttctgatttcttgtgttttgtgactgtgatgcccatgatacatgggagggggagggggcaatttgaaaggaaaggctaagacacagaagtgacttaggccatttcatccatggtagttatcagtggtcatctcctttgtgggatacccttggcttcctcccctagccctcctcctccttcctctggcagccttgagagcatcaggtggatgcatgagccggagcccgcatgtgtaagaacaggccttgctgctcctactgtaagtggactgagtgacaaggaggctttttcaaggtttcctcttgactgaaacattctcagattctaagatggcaatgatggtgtcattccaaagccaagcagctactgtttgatatcactggtccttctttaagtcaggccactgctaccacagcacctccattttaacccaaatgaatatgatattacaaccttactctgtagctctcactgatttgctgtcttaccacgggggcaaatctctgcacttgtagctttccccaaaatgcagggcgttcttctgcccaccataaaagatactataagaaactgtacgtctttggccacttaacagtacaaggcatcattgcggtgatctctttgtgtgtgtgtctcctaactggatggtcagttccctggggggcagtggctgtatccatacttctgtgtattcttcacggcacctaatttttgccctataaattgcaaaggtgctctgtgaattcagcccagcacttcatgagttatgcatgacggggatggtgctgctgcctcagagcattgtattgtgtataaaagtaaggtgttaaatattcctacttcattggtaccttacttactgtgggatcagagaacacaacaattccgaaattgttctcatagtcaaaacaatagtatttttaaaaatattgtaaaaacaatttttgaatgctcaccacgtgccaagctccaaggtaaatatttacatacattatccatttccatccatcggaagaatggacttagggattagtactgttactattcctactttacaggtgaggaaactgagccttagggagggaaataacttgtccacttttgcacagctagctaaatggtggagttgggatttgaacgaagcagtctgattccaaatcctgagttgttagaggtctatcttgatctctgttttctcccttaataacttaagataaagaaaatcaaagtgcccctgggctaaccaggcagggacttagttatctcaaagaacggggaaaaacatgaaaccactatcccttccagagagtaactatttaataaagaaaacattattaatacccccaggggagtaattaaaaagtactcatgaaacaagtagatgaaatttcaggctgtgaagttcaaacagttctggagtgaaagcttcttgcacagggtcatttggaatggtccactaaaccatagcaattaaccttggacttctccttggatgtcagctggtgacgtaactcggtaacgcatgagcttgtttattggacagaattcttgcgagatttacccccaaggtctttgaaagctctgtcaagaaaaaaagggacagcagtctctaggcgttctttttttcctgttgatccatggaatagtgccaatgaaaagtcataccgtagttattttttgagaagtaaatggtgattgagattcgtgggtaggagagttatgctataccaataaacgaatcaggtgcctcgaaagtgacatatattgttcctttaagcattttttttaaaacagctctcagcatgttctgtagatacttattattttccagcccaataattatactttttcattgattatgcttatacaacaaaaatggatagagtgttctggagacaaggccagtggtgaaatgccaaaatacttcattttacagaatgttaagcatctggtcatttttctataagtttcttgtaaaatgtttcatcaaagtggaggggtagccacaaagggaggaatttcattttggtaaccagaaccagcttatcccatcctactcacttcatcatcactaccctggctttgtaaaacctgttttgccagcttaggagggggcttcatactgggcaaggaaagcagagtcccttgcagtgggttttcaccatccaccagattgaagcacattctgcaggctgtctgcatatcataagtatggttataatgactcacaatttaaaattctattcaccactcaatcctccggcaccatgtagcatcttgcctttgtccatttggcactgatacttgtaattaacaaaaggacccatgtaaaccatgtgttttttatcatatgcctttgaccagaaaactcaaaacagacagcatccaatctgtttgcaacattagggttgggaaggaagagtgttcattctgttctctctgtttcaaagatgcagtgagatgggctagaggggacttaatagacacatgtgcaagaggctaaaggtgaagccaaaagtggacagagatatcccaattcctgttggcccagctcttctcttctatggaccatgtcctcttaactgggatccaacaaagggtcctcttctcatcccttcctcccttatactttttaaggcataatgggtgattgagaagaaatagaaaagttaatacattatattcattaggatagtagctcaatttagctttatgtttattttttgagacagagtgtcaccctgtttcccaagctggagtacagtggcatgaagatggctcactgcagcctcgacttcctgggctcgagtaatcctcccacctcagcctcccaagtagctgagactacaagggcgtaccaccacacctggctaatttttatgtttttaattttttgtagggacaagatttcaatacattgcccaggctggtctccaactcctgagctcaagccatcctcccacttcagcctcccaaagtgctaggattacaggcatgagccaatcgatttatcttttaaagttgtaatagactgggtgtggtggctgaggcttatgcctgtaatcccagcattttgggaggcgaagatgggaggatcacttgagcccaggagtttgaggccagcctgggcaatgcagtgagacctgtctctaccaaaaaaaaaaaaaaaaaaaaaaaaagttgtaatagatgtggttctttgaggaggtattttgagaaaatatgcaaatagactttgatccatgacttttcttccactggccatgacctgtgattaaattccagcataaaagggcatagcacaatatcatgtctgtgaggagtaaagccatgcattaaagggctgcatgtggacttcatgaaaagcgtcgctgtgtctacactctctttaatgtaggtttggagagagaggatgactttggttggagtactttgggcctggttgataatcactaaagatagtaatgagtgatcatttatcccagagttgcaatgccttcttgtatcatgctaggagccctgacagcctatgggtgatgcaaaacgaaagaggatatatggtgtcatctctgggtgatgctgcgggggtgaggagagtgaagcatcacaagacaagtgcccttttcagatgatttccaaaggaagggagaaaagggaagtaagagtgtgacttcatataaaagtctactataaatagactttataatattgagaagagccccagctggggcagatcatgggccatccatggagtgttctgcttctgacattaacactaaggaaactgttggagagcaggttaatggcttgcgtgaggccacttcaaaagttcaaggctgtcttccgtgtatgttgctaaacttctttttggtggagttatgttttctgtctctaccatcttgtgtgataatgagctacaaaaccagggatactgaggagagcagagtgccttaggagggcctagagttgataagcggttggggcagatgtaatctgtacagccagagaccttcatagcccatggaaggagccagtactgaacacttactgtgcttccttgattccagaatgattctgttgtaaggtggatttaagaacatgttttaggacaaaaaggaaacatttctacattaaatgtagaaccattgaattatgaaaacaatgtatgttagaattaaaaaaaaaaaatcgtactgtccccattggcacctatagtacttgacctggttgaatcacttttatgggctcctccctaggtcaaaccatgaaagatgtaaagttgcttttcagatgtctctcatatttacactttcattgtttagtagatacttctaagtcccaaatgtgtgccccatcctgggcctggcattggccatctcaggatcaatgtagaacttttgccagaggaccatcttgagcaaaggcctgggaatccactaagactttttgggaaccattgaggtaaccagtgatgtagaagggagacttaaacagcagatatggctgagagataacattagaaagtaggctagagacagattgtgaggggccttgaatgcccagcaacaatgacttgacctttatccttttggcagtaaggagccattgaaggattttttgtttgtttgtttgtttttgttttttttttttttgagacagagttttgctcttgtcgcccaggctggaatactgtggtgtgatctcagctcactgcaaccccctcttccaaggttcaagcgattcccctgccttagcctcctgagtagctgggattacaggtgcccaccaccatgcccggctacttttttgtatttttagtagagacagggtttcaccatgttggccaggctggtctcgagctcctgacctcaggtgatccacctgcctcagcctcccaaagtgctgggattacaggcgtaagccaccacgcccggcctcactgaaggattttaagcaaagacaatggcataatgcaaaatatgcctaaagcaaagcatatttctcctggtgttggatagaatatgattcatcttagaagatgagtctcagagggagacttcattcttttccttcttttcctcttggtcaccagtcctgtccatgtagttctgcggaggagtgggcaaggaagaatgaggccgcctctgagtggctatagaagaagtctcatctagatgagaatggtggatcactgagatttttggacaatagtggaacagagcacaagttgccaaaatcttttagcttgataatggggagggaggaagaaagcagctgagagttaaattgaaaaaaaaaaaaaaaaaagctaaacaaaaaaaccaacttgttttccattaataaaagggggaacctgagtcacatgaggactggattgtcttagctacgtacttggcaatgtcactacacaaagaagaggaagtttggagaaggtctcagtgacataagggaaagttttatgtagggcaagactaaaagcagattgattacctaaaaaaagtttcctccctctaaagatgtttccgtaatcccttcctggctactcctggaataaccctaaattttgtatcaacaatcattagctcaaaatagagctgggcagaaaatacttccctaagattcttttatactcataagcatgtttttgtttttcattttgttttgttttgcactgaggtgtatttgggtaaaatttccgtgtgtgtcatgtgggactagtacagacttgggagcccaaggcttgttaatatcacttgatgctttcttggaggaccagtctactgcatatcccaaattgggacaatttggagaagtgttccagttcttagcttccagtggttgccagcagtcctcggggttaccgattagaatcggtattaccgatagaattgaggttaccgattctagaagagctggtagctgcctaggattatgggtccacatagggaaaacctttaggaaaagaaggatgctggtttccataaacagttcataatcaccttggaccagcagttctggagaacagaggttctgattcaaatcaggccttgaggtctcattccccaaggagtgggaggcatgtaagcccaggggacaaagcaggactggcctcgaggctggagccatgtgccaatagccccctacgtaccaaccttatttacatggtggtgcggggtgccttatcattaggagtctttcagttgtgagggattgtaaatccaatcaaaactagcctaaagagaaggaaatatattggcttatatataattggaatgggaaaaaattgaaaaatcaaaatacagttcacatttcagttatggatggcgttgtggcttgaattgtgccccccccaaaagatcagaagttctaatttctgatgcttgtgattgtgactttattttgaaatagagtctttgcaaatgtaatcaaattgagatgaggtgctacctgactagggtgggccctacttcagtatgaatgatgtcctgataggagaaaacacacactgacacagacaacagggagaaagctatttgaagacagacacagggattggagtgatgtgtctacaagccaagcaacgccgaggactgctggcaaccactagaagctaagagaaaggcacagaacagattctcccctaaagccttcagagagcttggccctggcaacaccttgattttggacttctggcctcctgaactgtgagagaatacatttctattgtttcagccacccagtttgtggtgctctgcagccctggcaaatgaatatagctaggcttagaggttcatgaatgtccccaggacttggtgactttccatctgtcaactctgccttcctttacattggttctgtgtccaacctctacatagcagccagatcacagccagtaactacagagttggacaagttgcacatcctttatctgaaatgcctgggagcagaagtgtttcagatttttggattagggatgctcaacctgtatatccttccagaagcaagtgcaaaggagaaggttgtgtttctcttcaaatatctcaacttatgtctgattattctcaagggactttgactgggtcacgtgcctatcagagccagtctccatgatcgtggggtaccaggcctgagttaagttgcctcctctagaacctaggtgtggagttcttcagaggacatgaactcagagcttgtaatggtacctcttccaggtccagcaggccccacgggatgctaatagaagagagatgattggcatgaacaatgaagggtccaacattgccttcaaatctcagttccaaaggggttttgatacattattatgatggtgctttaaaaaatacagaatgttgtggatattttgaagacatcatatgtggaaaaaacagtttctccctagagcagagattgggacttctaggacaactttcccagaggagacgggaagtgtcagtggtaaggaaatgacagagtgggtggatggtgtggaaagctatcacagacaagaataattttattaccagcattaccaattatacagcacttttcttgttttctcacttgattttataataaccccatgagcaagtaagggagctccagatcacgaaatggggctcagaggtgaagtgacatatggaagatgacccagctaacacatggagaaactgggattgacttcagacctttgattccaaagctagtgctcttgtaactttctcactctttctaaaattcacgcattcattcagtaaatactttttcaatacgtcttatattcagggaactatttagtatgcagaatgaaaccttggttataaaaaaaaggagagagagagagtagtaacatcttcagggctttctgtgtgatcgatatgatgcttagggtctgtatacatcatcataagtatcttcacgccagctcagtgagatgtgatcacccccagattccagtggagctatccaagcctgagagtggttcagtcagttggccaagagcagaggtagccgtgggagggctggggtttggttccagctcagtccaatgccaatgcctgtgctcttaattatgttgcctctgctatactcataactctgttaacagccataaatccagctctgtctgttagacccagtaaatttcaaagtagaaaatcatttttctaataaaactacgcatagaaaaaaagatattaatgctcatacattctaccctcattatgacatcaacctctgagccaaactattttgcacattataaagagctgtttttatgatgaatgggaattatattggcactttaattgagttagaaaccaaggtacatgaatgttagtgcacaagaaatgcgataaaaaaagctgctcaatgtggttggaatacatcagattaatttaataccaactttaaatccttacaatctatacccttaaatatgttttatcaaattattagatgaaatttttatactgtttttttttcttttaggtaggtacctattgcacattccccccacccctgcttttatttttttatttttttttaagacggagtcatgctctgtcacccaggctggagcgcaatggcacaatcttggctcactgcaaccttcgcctcctgggttcaagcgattctcctgcctcagcctcccaagtagctggaattacagatgtccactaccacgcccagctaatattttgtatttgtggtggagatgtggtttcaccatgttggcaaggctggtcttgaactcctgacctcatgtgatccacctgcctcagcctcccaaagtgctgggattacaggcatgagccaccgtgcctggccctgcattcttaacaaatctgctatatgataaatttagatttcaattttgtgatcaaaactctttttttgctataaaatgaaactattgccctcttagcttcaaatatggtaatgtaggaggttggcatatatttggataaaattatgtaaacttaaaaaaaaacactttccacaataggatgttttaatattggttcagtttcagccataattaatgatttattttatgtcttttgttttagttcaaattagttcatcattaaaaaaaaaaaactgactccattcagtgcccatacaattagtacttgtgtttgcttgatttagcattctgcaaatgaaagagagtttgttttaatttagggcctgtgctttccttaaggtcaaatctccatttgagagaaagaatatggtatttaaataatttagtcaaattggaggccttgagacaagtcagagtccccaggctttctgaaaatgagatgtcccacgtttgcacttttccagcccaaccaaaaatgatagagttgtcagcataaaagttaatgtacaacatgtggatttttaaaacatgattgggatgagtttttgagtaattaatttgctgaaattgtgttgtgctttagcgcactgtactacaatattagcattgtgaagcgtgcattaaatagttcctgtcaattatggttggctgtgaatgaatctgagggttccttttgttataaattactatttcctaaaatggttttgcagagaagcaatggaacacttttagatttggaatgtttaaagagctgttcttgccagtggttgattttgagtgagctccaatgtttatgagaactcataaaacaaagcaaagtggggatggcccatttgctgttactccttttcctcccactgaaatttccctccagtttttggtggtgcctctgccacagttagctcatctgataaagcagggtgatagctgcctggccacgtatctgatgataatgatatgagcttttgcatacggtcccttgatcctgctagggccccacccccattctgagcatgcaacattaacataaaaactaccacgccttttgcagctgtggataaaccccaaattccacagctgggggtcacaagagaaagtttagctgaaaatgtatatacctaaaactggaagttagagggagggttatgaaatatttccaggtgcaatgtatgaatttacagggaattctttttgctgtagttagttattaggcaaacagcgctgttcattggtttggcaagagttcctaggttttgcggatagttctctgggtcatttaggaaaaggggtgtttggaagatgaccctgtgagagttgagatattttgccatgatcccctggtggcagcacatcagaattctgcaggtcgctttgaggttctttgttttgccttctcccttgattcttccttctgttcttatggctcaccctgcctttgttttgccatttaaaaataactagcggcccactgacggttttgccagaggcccttggaaatctaaccgtcaaataaattttattggtgttgctgctgatttttaaaatgaattctctgcaaataggcagaagttactgccagccagttttgatcaccagcacctttttgcttcaacagttcccagcagctaacacaataatggggccatctttatgtaaatagacacaatagtttatgtttctaccagctccagaggggttcacagtgttgatcttgactttcagatgggcctttctgagctgagggaggggttgctggatgggagaggagcttcccaggagaaaaccatgggtgaataatctcaaaacggttgttgcagctacactcgcatttggaggttaatttagaaaaagaaaagcaagattggacatcggaatggggactgcagggactgggccgagctaattatttcaaactggcctttcaggccatcctagacacagattggccctggatgggcctcggtctctggtctcttgaaagcccttgcctggtaggaagaagccgctctgccaggcagcggaagggagaggcaagcagtgtgagcccatgacgaggcttcagtttatggtttacttaggcttgaaaagggaaaaatggtgctaaattagatgtgttctggaatcagatggacactgttagtttcctctaaatttccttggccccacctcctttttgtgctttatttttgcacacctatgggccccagtcttttagcttcctcccatagattcttgattatttaggaaggaatctttccacacaaaaaggaccatcaagaaatgggatttatgtccgcagactcggcctgagaagagccgttcatctcagctcagggctgggagggagctgagcaggttttcttgcaggagcgatcaatctgccaccagatgtctctgtagcccactctacaggaatgctcacaaacaccagggctggagcctgagctttccggtgaccttgtggtatatgctctgaattaataaatgaagcagaaatgactgtgtgtgtgtgtgtgtgtgtgtgtgtgtgtatacgagtgcacacgtgcccatgtgtatgtattttctttcctgagttgcttctcagagtattcccctaactccttggttatctctttccctacactgagttccttcctaaaagtcagagaagagttgtagggtgctcccagaacgggagattcatcattgataggtgcaagcaaagacagtggcagtgggccctgataatctctgtctccttccctaaggtggctcttgggtgcagttatcatgctagggacaggtaaggaatgtcacttaatcctgggctccctgctggtccccagccaaccagcaaagggaaactcaggtgctgctaggggatgtcattgttgaagggctgcccaggaaggctgaaaacaaggatttgctttactgcatgtgtacattcattttagaagctttaaagtatttcaatggaggagcaacttagcaagttaattaggcaaattaaaaatatgtctaggaaagagagaattaatggtgaatgtggtatgagcctaatctatgcagtgggagatgctgtggacactccaccagtttgatcacaaagaattcacagaaagcaagccgcgcacggtggctcacgcctgtaatcccagaactttgggaggccgaggcgggcggatcgcttgagtctgggagttcaagaccagcttgggcaacagagcaagacactgcctctaaaaaaacaaacaaataaaaattagccaggtgtggtggcacaggcttctagtcctagctactggagaggctgaggtgggagtaccagaaggttgactcaggaggccaagggtgcagtgagccatgatcaagccagttctctccagcctgggtgatagagcgagaccctgtctcaaaaaagaaaaaaagaaagccagccagataaaatgtctggctaaattgggcatctccccaagtccggctggtctgtcctgagtacagtgaagtcagctgttactgcccttccatgtggagtttgatatgagcagcaaatgatctgacacatacactctctataaagcatgcttctcagctgtctcactgccatagtacagagaaaaggtgtggcacattcggcgacgtcaggcatgaccacacagagacacagcccttgaggaacaaggtgactgttcgcaggaggcgttgctcatctgcttatctgattttagttgaattgtctggcaaggatcataacagatttaggaatttttccaaataaaaggctgggatacaaaaataggaatcattcagtgggtgagttggtatctgaagaaaacaagagaacatttaatacagaacagtcctatctatacatgtatacatagacacaaaatataatccagcaagattcacacacagcatatcactgtcatcaacagtgactctctctccctaatatagggtggaaattgggatacttatgatagaatcatgagatgtagtcctgatatattccgaagatgtagccttgggaattttcatagatctttcctcccccagaggtcacacacacacaaaagcatcacgtcttgttttacaaacataagttgaggctggatcttctgaaaacaaaatggaaacattggtgtcgagttggagtgcttgcagtggaccgtgatgcgctctgattccttcttcacagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggtgaggtgacctgctgcacttgttttcttcttgaacaatgcatgtgtacttcctttaggtcctaaaatcgttcctctcttttggagtgtagctctagagctttggatcacatctgtggctgaaagtggaaatccgctgcaagcatgtcaccattttctctttctgtggcttaaatgatgccttttgtttgacttttgcccaacacttgttaggggctgagggtggaaatgataaaaatgtggtcacagagcccctgattccgtacaaccgttgatttctccttctgtcagggatctgaaaggaattggacttcgggtaatattattacacctgcaagagtacagtccctgtttaagggggcagtgtgtgctttttgcttagtgttgtatgcacacacctcccttaggccctcctggatctccagcccttcatcctggtttctttgtttcctggtacttagtacaactggcatgttatgtatggattgatttactgtctgtctccccaagagaacaagaacctctgcgttttcttctctgatgtatctgggcacatagtaggccctcaatgaatattcacctgaatgagaggaaccttgcagaggagagtggagagggcaggcatgtcctgcagggagtggagagaaaatgaagagaatagctgattttctctccttttcctcttccatggcgatattgcctacaacttaaggggtcagagtctacagtcacttagatctggctcaaatattaactctgccttttgttagtgtgtgaccttgagcaaatcacggattgacactaagcctcagttctctaatctctaaaatggaagtaacgtctacaacataggcttgttgtgaaggttaaatgagaagttgcgttaaaatgctgagtgtagtgcccgggatagactgaatgaccaatacatattagggaccatgaggacaatggctctcattacccacggctgtgagaatccatccctcgactgctgcacaaaatgtcgaatccattttcaggggttgacatctctggagatctagccattggctccaatggcagaaccccctccgctcacttgcactccacctgctcctgcctggggcatcaagcagattctgtttgcaagcacactatagccaaagctcaacttgcttccccaaacagcacattgggtgttgcacctgagtggggagaggcacctcccttcatgtctgtccctgggctaaaggcctcgctgctcttaccctcccttcgtgctgcaccaaaccctttaacagccctgagggagttgttcttccacccaaccatgctggcacccttgccgaaagagcttgaatgattctagaaaaatctgttgacgtatttggcaatatcagggcagctcccctgcttcctttcatagtccctgaaacctcctgaggtgaggacacaccacagtctacccaacagtgatgaagttaagataatttctggattaacaagtggtggttcatctggtaggaggacaaaataagccaggaaaggcttgacatccgaagtgcaggcagacaggccgcaggataagcctggaccagctgtctggctcagccgccttggtccttggtccttgaccctctctgagcctcggctctttcatctgtaaaatgggactactcaggcctgttctgaagattcggaaagatgccatgtgagagtcgcatgcagccagacacagagcgacagtgcgcgccggctgctgctgtcaccttcactgtcactgttactgtcgttcatcctgatggtgggaagaggagacaagcaggactccaggaccaaggaacaaagcattcttagctttttttcatggtagaaaaatcctgttaaaatggcttcacatgtcgcttacttttttaaaggatgccctgcacagcgtaaagaagagacttttaagaaatacagagcggagattgtcctgctgctggctctggttattttctacttaacactcatcattttcacttgtgtaagtatcttcttaaaacatcttctcttattaaaagattacccagggcaccaatccaagtatctcttgcagatagtgcgaatcatttaataatggtgagagagattattctttgaaccctggactttttgaggcccctagactgggagaatcattacaggaagctccctgaaatatttccagcttttgtctagtggctacgtttagagcattgtggaaaaaaaaaacaaagtaagatataggaaggacgtttgggaaatgacaaggggttctatgcaagagcagaggccctgtaggcgcagtgctagaagttgcagcgctgagggtcccccatcccagagcagaggccccgctcttcctgtgggtgagggagtgggccccactgccccagggatgccaggggatagatcagcctcctttggctgccttcaaactatttctcgtgggggttctccccttctatttttggtatttctgcccatgccttaagaattaatcccaagaagccagagcagtgaggcacagtgggaggcttccggggtgcaggatggctggccggtgctcaggcaccctagacatgcccatgagctgttggttgcaggttctggctcaaagccctcagagattctttctgcatggctgctcacctgtgttgatgatggttgtgggagagtagggccacatgtgtgtctgacccctctaggaagtgatctgccccctttgtctccatccaccaggcagggctggctacctaggggccaggacagacttcacccaggagctaccccaggactggttcttgccactcactgtgtccctctattcacttacttgcctctctggctgtgcactcatctctctggtttctattttagataccagtcaatcagagactccagtgagcacctactatgttcaaggcattatgctaggcactgtacagggcataaaaaggtgtaagacattgttcctgccctcaaggagcttacagttaggatgttagggttatttgcgtataagaagataattagagttaccaggcagtatgttttaaacatgaatgactttagcttcttgttggaaaatgcctgcttctgtgggcattgactttccatacagagacctaacagtagggggtcgaaatggccacaatcagtgaatctcctggtccaagtttagagacgccagtgaaatggttggtacaaatcccttgtggagcgagtgaggcagtgagtatgagagcttccagaatgggttgtctagccagctcttagtgaatagagtttaaaaggaggtgacaactgctgaatttttccaattattcacttcacatttctttcatttctttttagaagtttgcacggctacagagtatcttcccagatttttctaaagctggcatggaacgagcttttctcccagttacctccccaaataagcatttagggctagtgactcctcacaagacagaactggtatgagcaggatttctgcaggttcttcttcctgaagctgaggctcaggggtgtgcctgtctgttacactggaggagagaagaatgagcctacgctgaagatggcatcctgtgaagtccttcacctcactgaaaacatctggaaggggatcccaccccattttctgtgggcaggcctcgaaaaccatcacatgaccacatagcatgaggccactgctgcttctccatggccaccttttcagcgatgtatgcagctatctggtcaacctcctggacattttttcagtcatataaaagctatggtgagatgcagctggaaaagggtcttgggaaatatgaatgcccccagctggcccgtgacagactcctgaggacagctgtcctcttctgcatcttggggacatctctttgaattttctgtgttttgctgtaccagcccagatgttttacgtctgggagaaattgacagatcaagctgtgagacagtgggaaatatttagcaaataatttcctggtgtgaaggtcctgctattactaaggagtaatctgtgtacaaagaaataacaagtcgatgaactattccccagcagggtcttttcatctgggaaagacatccataaagaagcaataaagaagagtgccacatttatttttatatctatatgtacttgtcaaagaaggtttgtgtttttctgcttttgaaatctgtatctgtagtgagatagcattgtgaactgacaggcagcctggacatagagagggagaagaagtcagagagggtgacaagatagagagctatttaatggccggctggaaatgctgggctgacggtgcagtctgggtgctcgcccacttgtcccactatctgggtgcatgatcttgagcaagttccttctggtgtctgctttctccattgtaaaccacaaggctgttgcatgggctaatgaagatcatatacgtgaaaattatttgaaaacatataaagcactatacagattcgaaactccattgagtcattatccttgctatgatgatggtgttttggggatgagagggtgctatccatttctcatgttttccattgtttgaaacaaagaaggttaccaagaagcctttcctgtagccttctgtaggaattcttttggggaagtgaggaagccaggtccacggtctgttcttgaagcagtagcctaacacactccaagatatggacacacgggagccgctggcagaagggacttcacgaagtgttgcatggatgttttagccattgttggctttcccttatcaaacttgggcccttcccttcttggtttccaaaggcattttattgcttgagttatatgttcactgtccccctaatattagggagtaaaacggataccaagttgatttagtgtttttacctctgtcttggctttcatgttattaaacgtatgcatgtgaagaaagggtgtttttctgttttatattcaactcataagactttgggataggaaaaatgagtaatggttactaggcttaatacctgggtgattacataatctgtacaatgaacccccatgatgtaagtttacctatgtaacaaacctgcacttatacccatgaacttaaaatgaaagttaaaaataaaaaacatatacaaataaaaaaatcccgactttgggatgagtgctaggatgttgtaaaccagtttgagaatcagaatccaaaatgagagctgaaagattggctgagtctttctcggagggagggcatgctggcagacagagctttgtaaacagcatcctccttcccagagatgcttctgcttccatcctggggccacgttgctacccagtacatgagcagctcatactaacatgcacggtcatgggtgggcgggatggagggagggtttctgcttcagaaagatgtgtaacatcaggggctttgtgcctggattcatgggtttcactcaagattctcaaataggtcccttccccccaaaatgttaagaacgatgtggtctaagtagttgtaatagttataaaagcatcaggccaggcacggtgactcatgcctgtaatcccagcactttgggaggccgaggcaggcggataacgaggtcaggagatcgagaccatcctggctgacacggtgaaaccccgtgtctactaaaaatacaaaaaattagccgggcgtggtggcgggtgcctgtagtcccagctactcaggaggctgagacaggagaatggcatgaaccctggaggcagagcttgcagtgagccgtgattgtgccactgcactgcagcctgggcgacagagcaagactccgtctcaaaaaaaaaaaaagcatcataagtggaagtctctttacaaagatgaatacacataaaatgtctctaaaagctgtggaatcactttcaatggaatcaagtctgttctcaaatgctttaccaaaagtgccagggcatggtaattgagagttcacagagctcctagtcacctgagtgtgtagcccagcttcaagatttggaagttatatttccttgggcagaggacttacccctctaagccttagctggccaatctttaaaataagaatagtatctgcctaataggtttattgtgaggattaaataagataatatatagaagcagtaagcctagtgtgtagcaaaaggtaagcctttgactgatattagaacaagaaaggagaaaaaggtagcagagaaagtatcagtaaccataaatctttgacaaagtggttttgttaaaaggaatgaattggcttggtgaaggagtcatgctgctttcagaggattaatactcagtgtactaaaattcttcgtggccattagaattacagtacaggacacaccaggaagaagggttgccctttgtcagtttggactgaattaagctggaaacatgatggaaatttgagagcaggcggactcaatgtttcagacctagtctttggtataagaaaaagtttgtgtgtggcggggcacggtggctcacatctgtaatcccagcactttgggaggccaaggcgggcggataatgaggtcaggagtttgagagtagcctggccagtatagtgaaacctgtctctactaaaaacacaaaaattggccaggcgtggcggcgtgtgcctgtggtcccagctacttgggaggctgaggcaggagaatcacttgaacccgggaggcggaggttgcagtgagccgagatcgcaccactgcactccagcctgggcaacagagtgagactccatcttaaaaaaaaaaaaaatgtgtgtgtgtgaggcagagagagagagagagagagagagaagggggtgtagaagagaatggagggcagaatttgtcaaggagagtggactggtctcaactgcctcgattgaggcctacgaagatgtttcagaggaaggcagatgatcatggaccatatttattcttcatctccattgccagggaaagctttgtattcaaggctgtcccttgtctatgaaattagttctagagttataataattttgccttgggatgtcccagggcacaaatacagatgtgactatcagctccacattcttccaaaagaaagcctgtggttttttcgtatttataataatacttaggaggtttcctcgtagaaaatac

SEQ ID NO: 4 - DNA, 14016 bp, Homo sapiens
aaagcatctcataagggggtagacctatgttttttcagggagcagttcggactctcaacagggcaataggcctttcgactctccctgatgagggtggatgcacggcatgtggtactcccattttctttaggttgtttgttggtttttctgcgcactctgaaacgatctgcaacttgtctagcaagggtataaattcctacgcatccataaactctgaggactgcatcacacatagcttgggggccccagtgagttccttgatgtagctgtgacaatacctctcgcatgaagggcttagacagcatttccttcttgtctggttgtacccatcttccctcgtgactttctttggctcctatttcttttaacttttctttttctctgggggagaaaacagggactgtagctggagggggaagataaggggttaggtggaaaatgggtgctgcttgagaagaggcaacatgtttagccacttggtcagctagattattccctcagctctcaaaggaaagattcttctagtgacctggaacatgaacaactgctgtctcttctggcggctgtaagttctctagtacttggattatcaagtctctatggactaagttttggcctttactgttaataaggcctcgctctgcccaaattttcccaaaggtgtggactactccaaaggcatacgtggagtcagtataaatagttccttcctggttttgcagaaattttaaggcttgatttagtgtaaacaactcacatgtttgctcagaccagtcattaggtagggtgtctccgtctactgctgagtacctgttatgccttttcccttttattacttgagaagaaccatctacaaaaaggtgtcttccggtttgaaagggagtttatctaaacatctatgcccaagttcttctggtctggggcatgggtttttttctgcgtttgaatttcctgttaagaaggcagcagggttaagtgaatcatctgtagttcggattaaatcatctttttctaacaagatagccttgtattttaaaattcttgagtcagtaagcaacctttctgccttctgatttaggatagttctgttctggtgaggtgtgctcacaatggggtttcctccaaaagttatttttctactttcttctgttagcaaagtagttgccgctacagattgaatgcatttggaccatccatgggttactgggttaagaatttttgacaggaagcctacgggttgggagtggcctccgtgcttttgggtaagtactcccaaggctacgcccttgtttacattgacgaaaagatggaatggctgcttagggagggtaaagctaggacaggggcagttactaatagatgttttaacctttctaccttttggatttctggtaattgccaaatgatggggtctggctcgtcttgtgtgagctttttgtatgagagttctgtttctagggcataagagtctatccatagacggcagtatctgactaatcctccttcaatccattcaagcccaattttccatttgcctttgtaattaaatgccctaaatattttacttcaggttctacaaattggagtttgtttttcgaggccgttaacccttcatcccacagaaaatttaagacatgggttgagaaagctgctacttcttttctatcatctcctgaaattagaagatcatccatgtactggaggggacatatgcacgagggcagggaaaatttctctaggacttgttctaatatttgactaagtaaatatggagactccgtaaacccccggggtaagactgtccatcagtattgctgttttcaaccggagtgagggtctttccactcaaaggcaagtaggtcctggctgccctctgctaatggacaagcccagaaggcatcatttaaatctattactgtactatctgattaatagctctaaggtcttgcactaaccagtatgacctgtctggcttctttaaaggcagtattggagtgttacagggagacatacaggatttaagaagcccatcatggagaagaccttcaattacaggttttaaatttaccctggcttctaaaggaatagggtattgctttctctttactacttccccaggggttttaaatttaacatgaatcagagaaatctgtaactttccttgatcccatcttttgaccatacctcgggataaatgtgttcttcgtctgcggtggtgagcaagattagggaggggaggggaggaattttccgtgattgatttggaggcctaagcctaattttagtattaaatcacttcctaatagatttgtccctgcttctggaattaacagaaatttgctgctagctgatcagttttcatatttcacttttgtctcctctaagatttttgctctaaatccttctccttttactccagagataaaaagtttttcttgtgaacaagttacactagatggaagataacagactgaggagtgagctgcttctgactcgattaaaaaggtaataagcttaggtttaggtcccacttctaaatttaccaagggctgttggtgggactcaagagacaaagatggagcccctgacctccctagtcttcttcaaaagctgtaagtgggatgactttttcttctttttcccatttgggacattgtctttcaaaatgacctatttttccacatttgaaacatttgttctgtgatttcttagtaatatctcgccagctattggtgacaaagtggagctttaacattccttgtccgagggggtcttctgattctaggccagcgtatttttctcatttgctctttaagcctttctaaaaattctgtcggtccctcatcttttccctgttttatattaaaggccttggtaagattttgggtgcggggcactaattctcaaattccttttattaccatctccctaaggtctctcatatttccttgatgggctatattgttgttatctcattgaggatcctgggctgggaattatgttcagccgctggaacgttctgaccgggaggatgttcacgttcccgaatggtcatagcagccctttgtatcatgctcctttcttctcctgagaataagatgtctaagatagacattaactcgtctaaagtatatatctggggtcctaaaactgatcgatctgatctgccactccataagggtcatctaagagtggtttaagctccttttttaggtttaggggaagggtctgtggtggcagctgcttgggggagaggaaggttcttcagtccaaacaaaacagcagttttttatcatttgctgctttttcttgtgtttggtccttccattatccttccaatattctaacgttaggcctaggggactatcagagggtatattatcatgatcatgatatttcctatcttttgtcttacttgctgtatttcccatcctggagaaagagtttttccctgagtccatggggctcaatctctcttactagagatttcttgcaccctagtgagtctgtggggctcaacctctcctactagagatttttcacactcttcagcttttgctttatccttctccatatgcttctcttgcggaaattttcaagtccctcttagcataggcaggttggtataaaccccacaacaggcaagctgcctttaagccatatgaggtgactacagaaccagatccggactctgcacttgctctgcactcaattgtgtgtcttactcacacactttcaacctccaggatgtcctgaccaccaaggaaatacttcactgcccccaaggtttttcttaccttggtctatgcacagagttacctggtcgccacagtatctgtctgccttttcttccctcattgctagagtccaggtttattcatcacaccaggtgggtctcgatcccttacccttgaggccaccgcaacaaagcagcgggctgcgtctcctcacgagaaatgatctgagaccctccccggaggagaatgggaatcccagatgaacccccaagtttgttagaaacaagtgcctggtgccacaaagaaaaacagcacataggcagaaaattcctcagcaaggcaaatttacttctgcagaagggtgcagcttgtgctagtcacaatcgcaagagcacaccaagcagggtagggcaggggtttttaatccctaatgcagttcctagcacttctgtgtcctttccgcattggctggggttggacttcacaatctaagctaattcgattggctaagatttaaaattgaatagggtctattaggtgggaaggaagaggaactatccgttactaggtgggaaggcatatctggacttgtctgggcctggcgaaggcaggaaggctgtttacagaacaggtagctaggagacaaggatgtacaaggaagttggtcttaagaaacaaagaacagagaactaaacctttttgaagaggaatttatcatctctgacaggaggctgcagtgagctgagatcacgccattgcactccagcttgggcaatgagagtgaaactccgtctcaaattaaaattaaaattaaaaaataaaaaataacgtaaaataaaaaatggtttctctcccctctatgtgccagacaatgaggaaaagagaaaaaggagacacctctggaggccagggagctgagagccaccttgagaatgccaagctggggaagtgtttaggggaactacttcctgcttccttccgagcaaaacagtaaaaaataaaaatccctgagacaatacttccttagccttatgaaccccgaaaatctgagataggtctcagttaatttggaaagtttattttgccaaggttgaggacgcacacccatgacacagcaacaggaggtcctgacgatgtgcccaaagtggtcagagcacagtttggttttatacattctagggagacatgagacatcaatcaatatatgcaagatgaacattccttaggtctgggaaaggcaggacaactggaagccgggaggaggcttccaggtcttaggaagataagagacagatggttgcattcttttgagtttctgattagcctctccaaaagaggcaatcagatatgcatttatctcagtgagcagaggtctgacttcgaacagaatgggaggcgggtttgccctaagcagttcccaacttgacttttccctttaccttaagtgattttggggccccaagttattttcctttcacagcctactttcttccttccagaagtgactgtggacaattccacagggtttggacttgatcagggcagaaggtgaagctgcaaggtattagatgtgggaatggagaaaaatacaggctggagctgtgggtttgagtgttgtcctcataggaggtgatggctgaggggtaggtaagtgagaggatgagatccccgaggccgacagcacagagtgacaggagcatagggcaggactttgggtcacccaaggagacagtgatgcttttgaagaagtcagaggaggccccatcagcaatcagaggattgctctgattggcacctcagagctggaggacatcaaaaaataccgctgtaagaaagagacctggaaaagtctttagagattgtctatcccaccctacccatttgacacatgagaagatggaggccaagagatcactgagaaaataaatggtagagcttgggcaaaatcagtgctgcccaaaatggtgtttttccaacaaagacatttaaaaggttccttccacaaggatcaaacaccttggggttttgatttttatcttaaaaagttatataaatttagccttctacaggccaggcacggtggctcacacctataatcccagcactttgggaggctgaggtgggtggatcatgaggtcaggagatcaaaaggatcctggctgatatggtgaaaccccatctctactaaaaatacaaaaattagctgggcgtggtggtgggcgcatgtaatcccagctactcaggaggctgaggcaggagaattacttgaacctgggaggcagaggttgcagtgaaccgagatcgcgccattgcactccagtctggcgacagagcgagactccgtctctaaataaataaataaataaatttagccttctactcaagaacttatctggctttgtcttaatgtaaaaataatttctttttgctaaattattgagagaaatttactatttattagtgtttatcagttttctttaaactcaccactttttgatgaatatgaaaatctaaaaacttggccgggcgcagtggctcacacctgtaatctcagcactttgggaggccaaggtgggcggatcatctgaggtcaggagttcaagatcagcctgaccaacatggtgaaaccccttctctactaaaaatacaaaaattagctgggcgtggtggtgggtgcctgtaattgtagctactcgggaggctaaggcatgagaatcacttgaacccagaaagcagaggttgcagtgagctgagatggtgccactgcactccagcctgggcgacagagtgagactctgtcctaaaaaaaaaaaaaaaaaaaatggctgggcgtggtgcctcatgcctgtaatcccagcactttgggagtccagcgtgggtggatcacctgaggtcaggagttcaagtccagcctgaccaacatggtgaaaccccgtctctactaaaaaaatacaaaaaaaatagccgggtgtggtggcacactcctgtaatcccagctactcaggaggctgaggcaggagaatcacttgaatttgggagctggagattgtagtgagccaagatggtgccattgcactccagtctgggtgacagagtgagactccatctcaaaaaaaaaaaaaaaatcttaaaaactccttccagaagatttaatacttactttcacccaaccacccgacttgagtatcaccaataacagaggatacagtccgttttcagtagagccttagtagcaaagggttttcatttttatttttcagatacaggatcttgccctgtcacccaagctggagtgcagtgatgtgatcatagctgactgcagcctcctgagtagctaggactataggtgtattataggacaatttttaaaaaatttcattgtaaagacaggattccactgtgttgcccaggctgcaagtcttggcctcaagtgatcattccacctttaactcttgccctcaagcaatcctcccacctcagactcccaaaatgctgggattatgggtgtgagccaccatttccagcctactagcaagggtcttgttacatattacttggcatgatttatgtaatttaaaaaaattgtttgtttttcaaatagaaaagtaaaataacgaatatgcttttccaataacataatccccttctcacttgagaattttcctctaaaaagatatgctagatttatttcatgctttatgtgcctctggtgtgtccccttataacctcctccatatcatttagggatggtctcagctgcaagtaagaactgccacaacagtgatgtaagccaaaaaaaaaaaaaaaaaaaaaagcaaagccaagcaaaacaaagcccatttaattatttcccataataataagtctgggagaagaagattccagagttggctcagcagcttagtgacagcaaggccctaggctggcattttcttggccttcccgatggtcccaagatgactctcatggcctcaaacatcacttcctcacatcctgtcagggagaaagaggcaagtgagcaacaacaatttttgttgttttgatcatttgtcagagaggaagaacgttcctaaaaactccgcctctgctgtttgacatcctcatcctattccttggccatggtggtatctcatggtcactcctctatctgccactgtaaagaggaactggattgctatattctgcttagacacatgaggatgcagcccaccttcccagaacatgtgcggaattagatttctacaaacacatttgtcttgcttctgcccaactctctcactagaatgcacattccataggggcaaacatttttgtctattttgttcacagctatattctcaacacctagaagagtgacagaaattcaataaatagttgttaagtgagcaaatgaatgcatgaataaggaaaagggtacatggctattgagtaggtaaccagcagtgttgatcacccccaacagcatacaactccagtctgatgaacatcatgctactaagtggccactcatcacccaagtctctgaccttactttttctctcttttctcccagggagtgagccataactggtggctgctcttgcgccaatgagcctccccaattcctcctgcctcttagaagacaagatgtgtgagggcaacaagaccactatggccagcccccagctgatgcccctggtggtggtcctgagcactatctgcttggtcacagtagggctcaacctgctggtgctgtatgccgtacggagtgagcggaagctccacactgtggggaacctgtacatcgtcagcctctcggtggcggacttgatcgtgggtgccgtcgtcatgcctatgaacatcctctacctgctcatgtccaagtggtcactgggccgtcctctctgcctcttttggctttccatggactatgtggccagcacagcgtccattttcagtgtcttcatcctgtgcattgatcgctaccgctctgtccagcagcccctcaggtaccttaagtatcgtaccaagacccgagcctcggccaccattctgggggcctggtttctctcttttctgtgggttattcccattctaggctggaatcacttcatgcagcagacctcggtgcgccgagaggacaagtgtgagacagacttctatgatgtcacctggttcaaggtcatgactgccatcatcaacttctacctgcccaccttgctcatgctctggttctatgccaagatctacaaggccgtacgacaacactgccagcaccgggagctcatcaataggtccctcccttccttctcagaaattaagctgaggccagagaaccccaagggggatgccaagaaaccagggaaggagtctccctgggaggttctgaaaaggaagccaaaagatgctggtggtggatctgtcttgaagtcaccatcccaaacccccaaggagatgaaatccccagttgtcttcagccaagaggatgatagagaagtagacaaactctactgctttccacttgatattgtgcacatgcaggctgcggcagaggggagtagcagggactatgtagccgtcaaccggagccatggccagctcaagacagatgagcagggcctgaacacacatggggccagcgagatatcagaggatcagatgttaggtgatagccaatccttctctcgaacggactcagataccaccacagagacagcaccaggcaaaggcaaattgaggagtgggtctaacacaggcctggattacatcaagtttacttggaagaggctccgctcgcattcaagacagtatgtatctgggttgcacatgaaccgcgaaaggaaggccgccaaacagttgggttttatcatggcagccttcatcctctgctggatcccttatttcatcttcttcatggtcattgccttctgcaagaactgttgcaatgaacatttgcacatgttcaccatctggctgggctacatcaactccacactgaaccccctcatctaccccttgtgcaatgagaacttcaagaagacattcaagagaattctgcatattcgctcctaagggaggctctgaggggatgcaacaaaatgatccttatgatgtccaacaaggaaatagaggacgaaggcctgtgtgttgccaggcaggcacctgggctttctggaatccaaaccacagtcttaggggcttggtagtttggaaagttcttaggcaccatagaagaacagcagatggcggtgatcagcagagagattgaactttgaggaggaagcagaatctttgcaagaaagtcagacctgtttcttgtaactgggttcaaaaagaaaaaaataataaaaataaaagagagagagaatcagacctgggtggaactctcctgctcctcaggaactatgggagcctcagactcattgtaattcaagctttccgagtcaagtgattgacaactgaagagacacgtggctagggttccactggagaattgaaaaggactcttgagccctcctggaatggagctgtataactgtgcagagactttatccatgccaatagttgctgtccccttccaggggtcaccttgagaggcatgacagctgttccacaggggctatcccttctcagaaaacttctcttctgagcctctttaacagctttctccagaaccagtgtctgaaccaccctggaaattctgccttattatttcttactcaaacatgtttagagtggatagaaaattatgcagcttgcacacccatcgtctttaaccccaaatttcctttggctattaaaaaagtggtggcaaaagacatcctcaaaagaaagagaaatgaaatatttttgaatggttgcacgttaaaaattaaaagaaggaatgggggcagaatgccatatttttgagggctgtactaggtttatctcatttaagccccacaacaccccacaggagggtaattttctaactctagtttgcagaggagcaaattgaggttcagcaaggtgagagaggtacccaaggtcacatagctagttatgtgagaaagttagagtacagatcctctggggtttcagcttattgtagcatattttctccgaaaggcaaaaatgtgcccttttggccgggcatggtagctcaagcctataatcccagcatgttgagaggctgaggtgggcagatcatttgaggccaggagttcaagaccagtctggccaatatggagaaaccttgtctctactaaaaacacaaaaattatctgggcatggtggggcatgcctgtagtcccacttacttgggaggccgaggcacgagaattgcttgaacccgggaggtggaggttgccgtgagccaagatcacgccactgcactccagcctgggcaacagagcaagactctgtctcaaaaaaaaaaatacaatattttaacaatgtgccctcttaagtgtgcacagatacacatacacggtattcccaagagtggtggcagctcaaaatgatatgtttgagtagacgaacagctgacatggagttcccgtgcacctacggaaggggacgctttgaaggaaccaagtgcatttttatctgtgagttctgttgtgtttgtcaaaaagtcattgtaatctttcatagccatacctggtaagcaaaaactagtaaagacataggaacatgtagttttacttggtgtttatgttgcaatctggttgtgatttatattttaaagcttggtgctaaaccacaatatgtatagcatatggagtgcctgtacaagctgatgttttgtattttgtgttcctctttgcatgatctgtcaaagtgagatatttttacctgcctaaaatatgatgtttaaaagcatactctatgtgatttatttatttctacctttctgagtctcttggactaagaagatgttttgaaatgtaccatcaaatgttaacagagtttgatatgggctttctctttggtttctcatcacatttgtaaatgtcttttcaaaaggatttactttttgtaaaaagcttcattctcactctgctttgcatcccccaaacttcttgttcaaaacggggggagtttaggagactttaatcccggtttcagaagctgcagctggtctgtttccaggtcagaaaccattgttcagaagacctccctgtgagagagttgctcctcagggtccctcaggaccaaagaacactcgaaaagagcacttcacacagacaagtggctaagtgtccattatttaccttgaacaatcaaggcaactagtggagagaactgattgtgagctctgcctctgggtcagagagacctggatttgagtctgacaagaacaagaaatggtcaataaatataaattaccagcgtctaaggaacaaggtctatgcattattgtatacagtgtctctagtgcttgtatagtgtctggtatacagagggcactcctatgcatttttaaaacatgctgagcacataccatgtgccaggctttgtgttttatctaatgttatctaatggtattggtgccattatgtaatgttgcctttacaacaacctcatgagggagatttccatctttacaaataggcaaactgaggcccagagagattgaggaactgctccgaggtctgattctggaatgtgcttcctttccactttatcaatctgctcttcgtactcctgtctgaacgatggaaattaatttttgaatgtataaaagacaacagactatgatacagaaatgtcagccccagcccactaagaaagccccagcccatcagtggctaatggctttaataaattggtcatttggctacttggcttgtggacaatctctgacctcttttgaagatgggcactgcatggacttccaggaggtggatttaatagtcttaactcagcatgaaaaagatgctgggatgctcctggctatttatgcaccctaagtgccatagagacatgctgttggcaaggcatggtggctcatgcctgtaatcccagcacactgggaggctgacgcgggcagatggctggagtccaggagttcgagacaagcctgggcaccatggtgaaatcctgtctctactaaaaataaaaaattagccaggggctgtgacgcacacctgcagtcccaactacttggggggctgaggcaggaggatcacttgagcccagaaagttgaggctgcagtgaaccaagattgggccactgcactccagcctgggtgacagagagagactctgtcttaaaatgaaatgaaatgaaatgaaatataaaataaaataaaatatagaaacatgctgttaaagatcttatttgccaatatttatcattccacaatttgtcaggctttcaaagcctagcttgacgtgacatataattctcattgtggggagcatgtactcttctcaactcagatgcaagacaaatgatgaaggtggattgacctgaatcactgtagccttgaataagtgtcacagggcctcatgaccctgctgtgtctgagaacattctctgcctctttaagtctcctgggtctgcatctttcttaatgctccatggtcttggagccccaatggtctgcctatcccattccaggcagcagaggcaggtcttcttccttagcctcaccctatcttcctgctaacaaggaagcctcatttgtcgtctgagcaatcattagctctggtccccatatctattttggattcccagaccttatctgttaattaacaaatatttttccagcacttcttatgtctggcctgagccagaaagacatgatttcaaccctgtggagctacttcaggtttgcaagtgacagaaatcaactttgtgaatagttactagagtatcttaagtctgttttgtgctcctataacagaatatcacagactgggtaatttataatgaatagaaatttattggctcacagttctggaggctggtaagtctaatatcaatgtgctggcatctggtgaggaccttcctgatgcatcatgacatgatggaagagcaaagagagggcaagagagagcaaaagggagcaatcccactcctgtaataaagaactcactcccatgataacagcattagtgcattcatgagagtggaaacccatgacctaaacacttcttaaagatcccacttcccaatatgatcacaatggcaattaaattttaacatgagttttggagaggacaaatgttcaagccacatagcatggcatgctttgtggaatctagtgatactttggatgactttgccttgaggagggcttgagacaagtca

SEQ ID NO: 5 - DNA, 9246 bp, Homo sapiens
gatgtgtcagacgtgagaaagcgaaagtatgtcacagcgaatgtagcttttccacacgtatttcaagaaagaaatgaaaaagccaacttctataatggtgcctactgtgcattaacagagataaactaggggtctaagaactcagttttctacagggtcccagaagtatagccatatattgccccattctctaatggaaatagccagagaaatagaaatatcaagactggagaacatcaaatacctcattggaaaagcccccacataggaaaatgtgtgggcttgaattcttccattctggaagggtaaaggcctgagtgatgatgctgggattagacactgaaactctttagagaagcaaaacaagtataataaagctgtactttattatattaaataaataacacacagactaccaaatagcctgccccttataacagcgttaatgtgattttgatctgaaatgtatagagacattttgcattttttcgtataaaaagttcatgagatttggccctaatctgaccttttcttcatttttttttctacttgagggactataatctttatttttaaatttgttttatattctccgaacattacctaacgcatagaaaactcttattgaaccatttttctctgttctttgtaaaatattacatttgactgttccttagactgctttaatcattcctgcctatgcaccctcctcaaaatccagtttaaattaattgttccttattcaagattccttatatccacctcccttggggcagcaatcacctatcacccaggactacacttgtgtatgtacatatcttccctattacaaatcaggttctttgaaaaaatacaaatggtaagagagtggatttttggagtcagaacattctcttttcaaatccttcttctgccccttactggcaataagggctgagtgacctagagcaaattacttaacttctctgagcctcagttttctaatctgcaaaataggagccatcacttcacaagtctgtaagacttatattagactaagtgcctgcctgtacactgttctcttttctctctttctatatacctgaaggcattataggtgctagatgtctgtttaaagaccagacaatattgtcttaaaaaaacaaacaaaaacacagacaataccatctttaaaaaaaaaaaaaaagtccaggtaagaaataaataaggccatagaatggaagctttacaaggactctctctgagacaggatctcctcaagtgtccccaggttaaattagaagtatatatccgtacaattgttcagccagtttgtgcactgtactgaggatgaatgaacacctatcctaaatatcctagtcttctgactaaaaacaagatcatatttcataacgattattgttacattcatagtgtcccaggtgatttagaggataaataaaaatccattaaagaggtaaagacataaaaacgagaaacatggactggtttacacataacacatacaaagtctattataaaactagcatcagtatccttgaatgcaaacctttttctgagtatttaacaatcgcaccctttaaaaaatgtacaatagacattaagagacttaaacagatatataatcattttaaattaaaatagcgttaaacagtacctcaagctcaataagcattttaagtattctaatcttagtatttctctagctgacatgtaagaagcaatctatcttattgtatgcaattagctcattgtgtggataaaaaggtaaaaccattctgaaacaggaaaccaatacacttcctgttttatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaatgtaagtatatttcctttcttactaaaattattacatttagtaatctagctggagatcatttcttaataacaatgcattatactttcttagaattacaagaatcccaaactcaccaggatgctcacatttaagttttacatgcccaagaaggtaagtacaatattttatgttcaatttctgttttaataaaattcaaagtaatatgaaaatttgcacagatgggactaatagcagctcatctgaggtaaagagtaactttaatttgtttttttgaaaacccaagtttgataatgaagcctctattaaaacagttttacctatatttttaatatatatttgtgtgttggtgggggtgggaagaaaacataaaaataatattctcactttatcgataagacaattctaaacaaaaatgttcatttatggtttcatttaaaaatgtaaaactctaaaatatttgattatgtcattttagtatgtaaaataccaaaatctatttccaaggagcccacttttaaaaatcttttcttgttttaggaaaggtttctaagtgagaggcagcataacactaatagcacagagtctggggccagatatctgaagtgaaatctcagctctgccatgtcctagctttcatgatctttggcaaattacctactctgtttgtgattcagtttcatgtctacttaaatgaataactgtatatacttaatatggctttgtgagaattagtaagtaaatgtaaagcactcagaaccgtgtctggcataaggtaaataccatacaagcattagctattattagtagtattaaagataaaattttcactgagaaatacaaagtaaaattttggactttatctttttaccaatagaacttgagatttataatgctatatgacttattttccaagattaaaagcttcattaggttgtttttggattcagatagagcataagcataatcatccaagctcctaggctacattaggtgtgtaaagctacctagtagctgtgccagttaagagagaatgaacaaaatctggtgccagaaagagcttgtgccagggtgaatccaagcccagaaaataataggatttaaggggacacagatgcaatcccattgactcaaattctattaattcaagagaaatctgcttctaactacccttctgaaagatgtaaaggagacagcttacagatgttactctagtttaatcagagccacataatgcaactccagcaacataaagatactagatgctgttttctgaagaaaatttctccacattgttcatgccaaaaacttaaacccgaatttgtagaatttgtagtggtgaattgaaagcgcaatagatggacatatcaggggattggtattgtcttgacctacctttcccactaaagagtgttagaaagatgagattatgtgcataatttagggggtggtagaattcatggaaatctaagtttgaaaccaaaagtaatgataaactctattcatttgttcatttaaccctcattgcacatttacaaaagattttagaaactaataaaaatatttgattccaaggatgctatgttaatgctataatgagaaagaaatgaaatctaattctggctctacctacttatgtggtcaaattctgagatttagtgtgcttatttataaagtggagatgatacttcactgcctacttcaaaagatgactgtgagaagtaaatgggcctattttggagaaaattcttttaaattgtaatataccatagaaatatgaaatattatatataatatagaatcaagaggcctgtccaaaagtcctcccaaagtattataattttttatttcactgggacaaacatttttaaaatgcatcttaatgtagtgattgtagaaaagtaaaaatttaagacatatttaaaaatgtgtcttgctcaaggctatattgagagccactactacatgattattgttacctagtgtaaaatgttgggattgtgatagatggcatccaagagttccttctctctcaacattctgtgattcttaactcttagactatcaaatattataatcatagaatgtgatttttatgcttccacattctaactcatctggttctaatgattttctatgcagattggaaaagtaatcagcctacatctgtaataggcatttagatgcagaaagtctaacattttgcaaagccaaattaagctaaaaccagtgagtcaactatcacttaacgctagtcataggtacttgagccctagtttttccagttttataatgtaaactctactggtccatctttacagtgacattgagaacagagagaatggtaaaaactacatactgctactccaaataaaataaattggaaattaatttctgattctgacctctatgtaaactgagctgatgataattattattctaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaaggtaaggcattactttatttgctctcctggaaataaaaaaaaaaaagtagggggaaaagtaccacattttaaagtgacataacatttttggtatttgtaaagtacccatgcatgtaattagcctacattttaagtacactgtgaacatgaatcatttctaatgttaaatgattaactggggagtataagctactgagtttgcacctaccatctactaatggacaagcctcatcccaaactccatcacctttcatattaacacaaaactgggagtgagagaaggtactgagttgagtttcacagaaagcaggcagattttactatatatttttcaattccttcagatcatttactggaatagccaatactgattacctgaaaggcttttcaaatggtgtttccttatcatttgatggaaggactacccataagagatttgtcttaaaaaaaaaaactggagccattaaaatggccagtggactaaacaaacaacaatctttttagaggcaatccccactttcagaatcttaagtatttttaaatgcacaggaagcataaaatatgcaagggactcaggtgatgtaaaagagattcacttttgtctttttatatcccgtctcctaaggtataaaattcatgagttaataggtatcctaaataagcagcataagtatagtagtaaaagacattcctaaaagtaactccagttgtgtccaaatgaatcacttattagtggactgtttcagttgaattaaaaaaatacattgagatcaatgtcatctagacattgacagattcagttccttatctatggcaagagttttactctaaaataattaacatcagaaaactcattcttaactcttgatacaaatttaagacaaaaccatgcaaaaatctgaaaactgtgtttcaaaagccaaacactttttaaaataaaaaaatcccaagatatgacaatatttaaacaattatgcttaagaggatacagaacactgcaacagttttttaaaagagaatacttatttaaagggaacactctatctcacctgcttttgttcccagggtaggaatcacttcaaatttgaaaagctctcttttaaatctcactatatatcaaaatatttcctccttagcttatcaactagaggaagcgtttaaatagctcctttcagcagagaagcctaatttctaaaaagccagtccacagaacaaaatttctaatgtttaaacttttaaaagttggcaaattcacctgcattgatactatgatggggtagggataggtgtaagtatttatgaagatgttcttcacacaaatttatcccaaacagaagcatgtcctagcttactctagtgtagttctgttctgctttggggaaaatataaggagattcacttaagtagaaaaataggagactctaatcaagatttagaaaagaagaaagtataatgtgcatatcaattcatacatttaacttacacaaatataggtgtacattcagaggaaaagcgatcaagtttatttcacatccagcatttaatatttgtctagatctatttttatttaaatctttatttgcacccaatttagggaaaaaatttttgtgttcattgactgaattaacaaatgaggaaaatctcagcttctgtgttactatcatttggtatcataacaaaatatgtaattttggcattcattttgatcatttcaagaaaatgtgaataattaatatgtttggtaagcttgaaaataaaggcaacaggcctataagacttcaattgggaataactgtatataaggtaaactactctgtactttaaaaaattaacatttttcttttatagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaagcctggatatttgttattttggaaacagcacagagtaagcatttaaatatttcttagttacttgtgtgaactgtaggatggttaaaatgcttacaaaagtcactctttctctgaagaaatatgtagaacagagatgtagacttctcaaaagcccttgctttgtcctttcaagggctgatcagacccttagttctggcatctcttagcagattatattttccttcttcttaaaatgccaaacacaaacactcttgaaactcttcatagatttggtgtggctatgaattctccaatatcttacaccctgcccagtgctgtgaggaggctcacctgtatggcctatatcaaaggtcttccctgccctttggctttccattgggtcctgccactggggagtgctggtaggaactatgaggaacataagagattcccttgactccctccttgtggagtagacccaggatggctgtgtctctcaagcaaggaacccagattacctcaaggtggcactctgggtactttttccttctgagtgattctggtaatcttcccttgtccctttaagcctagggagggtggtacttttgctgttagcaactccagggtacttgtaccatcccttgcagtttccctgaactctgaccatagctttttaaatagtccttttattaaatcctccttttgattgagtatgccatctatttcctgctgggactcagatacagtaattgtatcagaaatagccccagaaaatagaccctcaaaataggattctgggactgggttgttcatatattcaaggaatgcaaggataataggacatgggaaatctacggaatgtagtagcatcgcaattactgaacttatcatcaatggtagaatgggatgaaatgcagacagatggcaagatgttgtgaggtcaaatggctgtggcacttagttgctacagaaacaacagttataaaaattatgattattacctagattcttttgatgatgatgaccccagacagagaacaaaggaaaaaaaaagttatcaacatacaattaaaaacatacatgggcaaccagaatgcctcttcagcagctttgaagaagtcgttcctctcttcaaaattgccaaggagtagaagtaaaagggacccttctcattaaatacctcacttggaagatttttttttcacatctcatccactaaatcttatcttggtcagttttaaggtcttagtgctcaatgaggcattcttctaccaggtgccttgacttctaccagagaactgatgaaatggctgagactaccttttggccatttaggggttcttcatatagctgaaccaacaagcacgtaaaggaccaccgtactgagcagggtgactgattttgatgaaaagggggaaactcagtgccttctatagaaccgggcaacgactacacaataggtaatactatatttggaactcaggaaattcagtggggaatctcttagctttctatccttagtagtattggtcaatggaaaactgtaacaatccttaaaattcaagaacatcaaagactcagatttcacagaagtgagatatagagtataccaccaggtaaataatgccacccaaccaaagtaatggcagagggtaaaggggaacacgcaaagggtagtggaagaatgcagctgtactaaccaattttcatgactagctattgaggcagagaacagacttgagcagttttgtttttctccattttttatactttactatgtcaagttggacttgacatcgtctcttattctttatgtgaagaacactggtgatacctaacattttagatttcaggaaaagtattgctgaattgacattaccctataatgatacaataactgatgggatttcatgtgtctcctttgctaggaacacaaaccttcttcccaaaggaaggaagagagcacatgctgaggaatgaaggtgtgaaccgtgtatcttctactttt

SEQ ID NO: 6 - DNA, 27294 bp, Homo sapiens
ggtcttaccccagtcagacccaacacctcacttttataacaaatatttggtaatgcaccctttgttatatgaaaggagatatttgtggataatgtaaccccagtcttcatgataaacaaaaaggcccagctgatttccaaaatgcaccccagtttagaatcagtctggcaagtatcacatgaaatcctattggtatttgattgggatcacacagcatttgcaaatcaatttaagcaacatttctatttttacaatattgtggcctctagccccaataacaattatttctcttcatttacttatatctttgtgtctgggcagggtcctgccaggaaacagacggcatgttaaagtgagaaactgatgagttcagcaaagtgactatttatatatttggacaggatttaaggaagtaagaaaggatgatgcaacacttcagaggggagtctttccgacctcaggctgaaggagaaggaacgattactggaattcagggaggagagcatcaccaaacaagagcttcattagaggactgcagccaacgcagggccaggcggagggagccagagggaggcaggctctgctctccctcttcctgcccttcagtttccaaccacggcctcctattggccaaacccaaccagaagccagcgagcaaggggctactgatgaagcacatatagctcagcctccagagacacagaacaggataaaaggctgagacagtgggtctggtggggcaaagagaaagcttgcacctgccaggtaaagcattatagtccccatcctccccccaccaccttagttcttgtgcatttcccatcagttttcttccaaagcatttcagatctcactgatttgaatgggacctcttcttctattaccaatttggatcagtaattatttatgtatgagaaaactattgatttttacatatcgttgcatagcagagtggtttaataaggagacatttggtttttactgcctgtgtgggtatcacttgctatgtgacttgaggcaaatccaatatttcttctgttataaattccagtatttgtaaaaaatgggtaataagatctctattttatatagttttagtatttaatgagataatacatataaagtcattaaaacagtgtctggctcataaaaaaccctcaataaatgtcacttattactgtatctggtttttgagctgctctattgcaccattgagttttcagcccagtatatgttaaccctgatcattatctgcagaagtccccgtgccacactctacatcatccaaattctctccaggtggactaagtagattaaagaactttaaacataactaccatattttggctctatctacaaaatgtccaataatcagttaagaaaggaacaattctcttggggcccacactttgagaagcaaatgcagctgaacttttttagaggaaagtgagtgaaccaactggtagctttgccactgcttaaaaaccagcatcctttccagctgggtctaagacagaataaggtaaatttagatatgtctctaatatatctatagaacagtggttctcaacccggggtgtttttgccccttaggggataatttgcaatgtctggagacatctgtgattgtcataactggaagggggcagtgctattggcatctagtgggtatagagcaagggtgctaccaaatatcctatggtgcaacagagaattatctggtcaaaaatgtaaatagtgctgagggtgagaaaccctgctataaaaacgaaagaaatttggtctacagagttgtttggatttagacaagacgttgccccaatagtggtgatagaaataagaggaaccccgtgcttttgcaaagcccatatctggggtggcttaaataatcatgctcctccccatcccccgacctgatctttgtagttggaaactccagggctggctgcctgtagtctttgtgactacacttcctgcctcccatcacttcatctcagaagactccagatataggatcactccatgccatcaagaaaggtattttaaacattggaacacatatagataatttaagtaggtagatgtatgtgctgttataaggaagtggggaggagagaagagggaaccgaaatcatatgcacaaaaattttttttagaatataaataaaaaatgtggtagtctaaaatgtcaattcttcaaagataaagttaggctttcagtaacgttagaaatggttttctggaatatgtctccagtctacctaactttgaggaagtaaatactgtaaatagatgtttcaaacgcattttaaagcaatgatcctagcatgtctttaagctacagtattgtgctgtctttgaaatgtaaactttgatgtcttctctttctcttagttgatgctattgggcccatctcaagctgatcttggcacctctcatgctctgctctcttcaaccagacctctacattccattttggaagaagactaaaaatggtaagaacagctcagagaaccttaaaaagtgttatctgtaatctttgtggaaacaactgaaaccagctggcaagagcaatattgaagaatctgtacttaggttatttgctgggggaaagtgcttcctgatatttcacaattggcattaatgaagggggcatgtcacaatttcagattaatcaacgcttgctctgttcaacttcctacaagaattaaatatgtgctgtggggaggaggagcagatgtttgaattggggacatagcttctatgtatctcatttcttcagcctacaattttggctttaaagccataacaaatcactgaattactgaagttactttgtgctttttccagcatatggtgttgtcttaatgactgtgtggatgaaagtgtgtgggcaggctcatagcaataaaatacgggaaatccccgggcttgagtgctgtcaaagaaaactaaatttggacagtagataaagatactatcaggactattgcaatcggcagaaagagacctcagtatagaaaggggctcaattccaaatacagccaaagaccagtaaagatttctggccaaggagtagagtgggggtcagtggatggaaaattactaagaggaaacatcaagggtaaaaggattctggctaaaccgacctgacaggattcttgctgaagacaggccagggtgatcagacctcacctgtggatggtgggagatgaggaatttgatcagatattgagggtgatcacataccaagaggagtggattatcaataaaatgacttagcaggattcctgcttgaactgggcaatgcaaagatggacatgaagccaaaggccgaagcctaggggtgtagtagagcctgattaagttgaattaaggagagtctttgtcagcgctggctctcccagtcactagttgggggggccttgtgcctgtcatcaaagtcctctgaaactcaatttctctgactatgaaataggcattagaatccctcccctgttgccttccagggccactgtgaggctcaaataatagactatttttcaagtcctttgcaagtggtatgatgcaagtgtgagttattaggtatgccaaaacttagtcggaaaaagacgtcaagggcctttttctgaaattattttgtcacttaaatcagacacattctagatccgaatgttagctcctaggctcattttgtgtcaaagttctaatgaagcattaaccatggggctattgttacaaaggaaacaactgcttacggtttcatttcctagaaacccagatgtctattttaatgcaaacctatgcccacatctgtctttgccccttgatgggtggcataatgggaatgatagtaatacagagagctcacatttcttgaccactcaactatcatgctgagggctagatagacatgattctattttggcctcaaagtagccctataaggtagagataacgaaactggggctttgagaggttaaggagcttgggtggctctgaaagctgtgctgaagactcttctgttcttcctagaccaagcccagcacacacgcaataaagatgaggttggatatgatggcttcctactcaagtacaaaggggaaatagtatatcttttctaagaaaagacgtgaaaataattttcaatataagaaattcaaaaggcaaaaaagcacagggaaaatattcaactgtattgagtcatatggcagatcctttgatctagagattacacttttagaaactcttcttaaagaagtgaccatgagactggataaaaaaatgtggcacatatacaccatggaatactatgcagccataaaaaggaatgagatcatgtcctttgcagggacattgatgaagctggaagccattatcctcagcaaactaacacaggaacaaaaaaccaaacaccgcatgttctcacttataagtgggagctgaacagtgagaacacatggacacagggaggggaacaacactcactgaggcctgtaggaggagggtggggcaggagagagcattagggtaaaaagctaatgcatgctgggcttaatacctaggtgatgggttgatctgtgcagcaaaccaccatggcacgtttaactatgtaacaaacctgcacatcctgcacatgtaccccagaacttaaaaaaacaagcaataaaataattttaaaaaaacaaaagaagtgatcgtggacatggaaaactatttaccaagatggtcagtgcagccaggcaaaaaaaaaaaaaaaaaaaaaatcatgtcccatgttgggaaggggtgaattaattgtagtagactcattaaatggaatattatgtaatcatcaaatcatgttttttaaaataatactgaatgacctaagaaagcactcatggtataatgttaaatgaaaaaagcaagctagaaatggataagtaccgtgtattcctcatgtttttactgcacctgctaggcaaatactagatgctcactaaatgttggataatctgtgatgatggtttacataaacacatgtgttgcatattctaatttcattcaacatccctactttataaccattttacagttggcaaatcagaggctcatgaggtcaagtgatttatgaaagtcagagagctcttacatgacagaacaaggacttaaaaccaaatttttgtactgacaaagccttggctgttactagaatgcttctcaccatgtgaaatagatgcagggatgggaaattactattagaagggaccatctcccaaaatgtcaatagtggttcagcaaatttaaaagtaaaaatattattctgctcttaacctataggaaatttctttatggctaaaaaaaggttattaagtaatcaatttattaaattaatacaatctgattatttaaaaatttggaacgctgtactaaaattaaaaatcatcattacagattaaccagccagtacctctgcaccccaagaataaataatgtatatccccgaaactcaccgaagtttagggctggggttggcaaactatggcccatgggctatatcccacctgctgtacagctcatgagctaaggggtttttttttaattgttgtttttaaaagactgaaaaatatcagagcaaaattactattttgtgacatataaaagttacattcaagtttcagtgtttacaaatggttttattgtttgagtatttgtttacttattgttgataagtgcttttgcactacgatggcaaactattcaaggagttgggtagtgtgacagagaacctgatggcctgcaaagattaaaccatttactaactggccctttacagaaaaagtacgtcaggccggggcttatagaaaacaaagggataaggtataaggtcaaataggtttgagagccctatggtctttggtgactgttgtgatgcataatagctgttgagttcctaatttatgtaagacaactttatatccttttattcttttagtttgaaaactaagtctgttgggctaaaatgataggaagtaaatgataactctctcctttttttaaaaaaaagcaagtggtttacaaccttgtacttaaacgttttggtgacataatgaaactgatattcatggtatttgtactttacagagattaaactaaaattaaaaatatttcaaaattcacaaataggggatatttgttaataaatctatttgggaaattcctagcagaggctcagtctataaaatgaatagcatttcagcaacttcccttattcacagtgcttggttattctctagggagacatacacaacacatctctagttaccaaacaattcagtgtgatataaacatggcaaaaagtcaatgaatttgagggcaaggtttccagcaatcgccccggccattgcttacttcttccatgccctttctaagttttcttcagccaggcagccatcccctctggtttctcccagacccccgctgcaggctccccgccatcacagaaagcccctcgctcacacgtcttggctcaagcaactctttgtcttagaaatgcagatcccaacatttccttttaaactcaggcaacttggcttttttctgctctgtgatcttgaaagtcgcttggaggaacagctgagtgcatggggctgttgtcctctcagggctaacatgttgtagcccagggggtgcccaggggcctttctgactggttggttagttgggtaaaagagtagagtcaggagagcaggaaatcctttcttaactcactataaaaataaaagcgttccccaggcctcaaatagtctcatctcaagataaatttccttttgccaagattgctgctgaaaataatccattgtagccagataatagctatgcaaagaatatataatagactggcaggggcatgcctaccgattcaatacagaaaggtgagggtttcatttgctggggtgtagtgggtgggagaattccttattgcaatcacactctacttctccatccagaaaactctccaaccctcctggaggactctccattttctcctctttctcctccttgtgtacctacctagaccatctgctcccatatgtcctgtctgacttcctgttccagttacctatcactgcgtaagagatcacctcaaaatgcaatggcttcaaacaacaacaatcatatactgctttctatcatgggtccaggagttgactggactcattaggcagctctcccacagggtctctcttggggtggcagtcaggcggtgactgcgactggaatcacctgaagactcactctccaggtctgatgcctgggctaggagactcaacagctaggtgccgaagcagctgcagctcctcaagtgtctctgtctccatgtggtctctctaatatggtggttgtcgtatagccaggcttcttacaagggtgatgactcaggactccaaagcaagtgggtgagagaaagggagagagggagaaacagggagagagagagagaaagtgtgtgtgtgccagtacgcgcgaggtgaaagctgtattgcctgtgaactacccaccatgtctttcgtcctcttgacaggaaacctcctagaaatgtttgctgtctccaaatccctctccttacgttcttccaagaactttgaagtcatattttatgtagctactccttcaaaacatatctggtgttcggccagttcttacgccctccagcactgctacctgggacttctgcttgaatgactgtaatagcctctcaactagtctccctgctttcacccttgcccctcactgtctattctcaacacagcagccagcagcatccttctcaaatgtaagtcagaccaactgattgtcagctcaaaaatttgcaatgcatctgcattccacccagagcagagaccgccatccatggaatggtagagaaagcccaacatgctcagggacactccctctctgacttcatctcctattgttctcctacaccccctgcttcagcaatattggccccgttgccatttttgtgaatattctagcatgttttcaccttggggcctttgctccaggctaatccatctgtctggaatgcatttcccctggatgtctgttatggatgactttgtcctttccttgaggtctttgtttagatatcaacttcttaatgatgcctatccaagctgccctatttatcgtcacaatcctaccccacattcctgatccttttcactctgccctgttttctttttcagtaacacttatcacttgacatgcaatatcatttctgacagttatatatttttgtgattatttagagaacataagctatagttgagtggaaatcttttctattttgtccactgatgtcccaaacacctagagaagtacctggcatgttgcaggcatcaataaatacttgttgaatttttcctttttcacaatttccttctacgttgttatgatgagatcttatttcctctgtaatttgattttaaaagttttaataaaaaacaatacatattatttatgataaaaagtcaaagagtagagaagggtataacataaaaatagaagtccccctcttcccagggaaggcccctttataccactgcccagaagaaattgctattaaaggtttcttgtgtattctttcctacttttctctgcaaatacaaatatatgcatatatatttatcataaatgcattatatgttatatgttattttaatgctgctttaaaaatcccctttattttttgtaacttagtagtagatcatgcatagctttttatgtcgatacccacagctctaccacattctttttaagggacatttgatattttactattggtagtttcccatttttaaccattctctcaaatcaatggattgtcatgtaattcttcctattcttactatttcagaaagctgaatcaaactagcaaaatagttttatctaaagacatataaggccgggcgtagtggctcttgcctgtaatcccagcactttgggaggctgaggcaggcagaccacctgaagtcaggagtttgagaccagcctggccaacatggtgaaaccccgtctctgctaaaaatacaaaaattagctgggagtggtggcggctgtctgtaatcccagatactcaggaggctgaggcaggagaatcacttgaaccgggtaggcagaggttgcggtgatccaagatcgggccagtgtactccagcctgggcgacagagtgagactctgtctcaaaataaataaataaataaataataaagacatataatgcttactttaaagaaaaacaaaacaaaacatgtactagttatttttttcctccctctgtggaattcttagaaggtttatggtagtttgaagctttgcatggaccattttgaaacagcagcagcctgaggttccagggggttatgaagactcccagctgaggacagaccctggcagataagtttcagggggctctacaccaaccattagagtcatagaataagcacaatagaaaaggaccattaaggtcagttagccaaactccagagtttgttgatgagaaagtcaaggttcaggataattcagttggtagccctgtagcagacagagagactgaaaacaaatctgactttcagttcacgtggtgctaacccctagaataaataaacacgaggagaaatcagactaatcccagtcttcttctaacttgtcacaagacacaaaccacttaccttcacttcctcattttttccatctaatagttcccagttatatacatgtccttctcactcctctgattgcaaccagacatctcttacaagtttacaaagttttgaagataaaaacgctatttggaaagcgtaaagttaaaaacagcttggtaaatgtttttttttttttctattagtaattcgatctctacaactgtaaatattgtggtaggaatctaatacagatctaaaatcagtaaaattcaatcttgaatatgggcttcagtcctgccatcaaaatagtgcatccaggtggataggttttgccaccttgaagagttgtttattcaaacttttgtttgaagagtaggaaagcagtgttacctttaggcctgacttagcccttgccccacaatctattgttttttctcaccatagatttccctgacagcagagagagagttctgtgctcaagagatacacacagcttctgacaatagagcagcagagtatttggttcctaattgagcaggaatggtgtttgactcatcatcatttccctactttgtctagcacagtaccttgcacagagtagattctcaataatgtttgttgaatgactgtgggagcatataattcataatggagacaaagctcaatgaggctttaaatttctaaatccacaaaatgccctcatgtaacattgctggatgatatggtttagctgtgtccccacctaaatctcaccttgaattgtagctcccataatccccacgtgttgtgggagggacccagtgggaggtaattgaatcatgggggcgggtttttcccatgctgttctcatgatagtggataagtctcacaagatctgatggtttcataaacggcagttcccctgcacatgctctcttgcctgacgccatgtaagacgtaattttgctcctccttcaccttccaccatgattgtgaggcctcctcagtcatgtggaactgtgagtccattaaatctctttttctttataaattacccaaactcggatatgtttttattagcagcatgagaacagactaatacaatggacattggatgcaattcatttaaaaaatcatcttaaaaatatctttcttttttctccctcaagttggtcccactcaaaacataaacacaccatttttttttttttttgtcttgagacagagtcttgctctgtcacccaggctggagtgcagtggtatgatcgtggcttactgcaacctctgcctcccgagttcaagcaattctcctgcgtcagcctcctgagtagctgggattacaggtgcatgccaccatgcccggctaattttgtatttttagtagaaatagggtttcaccatgttggccatgctggtctcaaactcctcacctcaggtgatcctcccgccttggactcccaaagtgctgggatttcatgtgtgagccagtgtgcccagccaccattttttaatacttgtaaatttttcctataaaaacaaaccaatttctctatgccccaaaaccgctaagtagcacaaaatagaaacattagagtaccaagaatacttgaactgaaaaggaaattaatcaaaatgcagacacacattataccaagtgcatttgctgtagctgtgtaaggcaacttgaatagaattggtcaacaatgagtctgaatcttggtttgaaattgcctgtctgatctctgcttcctcatcagtaaaatgagaatatttatatggcctttcaacttcagtgtgagggatcaatgatgtaatataaacaacaagtctgccttagaacctggcacaccataagtaataaaaggcagccaatattttaaaaaatacacaaatcatggtctgatggctgtccaatataaattctctattttccattttaactaaagagacgatatattgagaaaatagaaacacctgtgtgtatgaaatcacccattcccatttttacaataattagtttgctaattgagcatccaaatttacccagtgtatttgcatgtgtaattagctgtgattcaataccaaagccaggcctatcatggtatactatgctattttacaagtcaaattactgaaagatgcatgtctttaggcaatcattacaaataaaaaaaaaaaaaccgaagcaaaacaaaataacatagattatttgtatcagatggacaaaacagacctggcttgatgccgaacccttaaatctcaaaataacgatagttgaagctaaggttccagcttaagtctgaagcaggtagtttccaatggcttgaaaggagaaatttctacactgaaggaaatttccattggaataaaggaatatttcacacttttaagtcatcttctctagatggtcttttgggtatactttctctttaaataacagatttagaagcactttgttcatttgtttagaattaattccattcacaagtttaacacagcctaaggtttggtctagaccaggggtctgccagctatgacctctgggctaaatctgtcccttcacctgctttttttttttttttttttccaacctgtgagctaagaatgggttttactattctaataaatagtgagttcatttttctccctcacctgcttgatcagagcccaactttctcattgcagttaatcttccttctggcatggatcttggaatgcaaacttgctgggatctccgagttccaggcttcccgtgcagccggtgtggagagccaagagatgttttgtttggcataaagcattccaagggtcagtgggcttgggctcaactattgagcataggacaagggcagccccatcctgactgtgactcttcccacaagagacaaacgagctctgtgctttcactggggtttcaggttcaaagggacagagcgtctgagaaaaaggattatgaaagagtccgtctgcagctccacttcccgtgcccttccaatgataccatcctcgtttcttctgtggcatgctccccacttcaatccttccttcagaggccccaaaccctcctggtctctccttgtcaccttgtgaaaatctgatcttcagggaaaaattccttactatttatactagtataatgtgaatcttctatgggattttaagaaagttcaaagccttggtttactcagcaaatatttagcttgcactcactatgtggcgggcatcctaatgatggagtatatgtaaagacaaaaaaagtttccggacctcaaagtgttctccatctataggggcagatgactgagttgacatctcgagaagtagaatagcagagtggctaagagtgccagctctgtctcaatcacctaggtctcacctcagcattaatttcactttcctcattgtaaatgagcatatctcttagaattgggataagcattaaataatatagacttggaatgaatttgcttagaactaattccatgcacaagtttatcacagcctaaggtttggtctagaccagaggtctgccaagtatgacctgtgggctcaatctgtcccactacctattgttgttgttgctgttgttttttaatgacctgtgagctaagaatgggttttactattctaattagttacattctcaatggttatttaagtacctccataatatcctcaattttgcctaaaatatttaccatctggccctttacagaataagtttgctgacttattggtctggaccaatgctatctaataaaactttctgcaatgatgaaaatggtctctatctgtacccttgaatacagcagccactagcctaatgtggctttttgagctcttgaaatatagttagtgtgactaagagattgaattttaattaatttaaatttatggagccacatgtgactatgacattagagcagctctagacagcctgaagtctaaagactctatgctttgtcggtgctcccctctctcaattgaatcaactaccctgaggctgcatgagtcaaggggaaggccacactcttcaatcagattttttgccctggactggctttcattgtctactagaaaatgcttaatgggaagtgcttagaaaatgtacatgggcatacacttaattaatctaagttgctgctttgtctgtatccattaaatctgctttattttggggtaaactacagtagaagttggctttttcaaccctgcaaagccttaaaattcaggatgtcttactcaacttaaagtgtagagttgcagccagagcacaactgtatttccttctagccctgcttgcagaatggctaacttcagtcctatttcatttctcttgtaagactgctaaaaacagtaagaagccaccaacatcattatgaatattgccaaatcatttcgcctaagagtaaagtcacagttggcatgtgttctgccctccaagacaagatagcataggtgacagttttatcagatatcttgtgatggcataatataggccacccagctttccagcctctgatatctgagtcttcccaatagcctgatgacatccgcatcacatattttaggttcgctcatggacagtaacttatttccaaattctatactggttaaaattaggtttgcatttgtgcaatagaaaatccaattgacattggcttagcataacaattttttgatttctcataaactcttggcagtcagcaggtccaagccattatttctgctctgctctctgaggtcatataaggaaggatctggatgctctgggtcatctacgtcatctaactggttgctgtgccatccctagctcatttttctcatgtgcattgcccaagatggctggctacaacatccacattacaagaagccaggtggaagcagacaggagaaagaggagaaaggggaactgccccaccgtttaaggacatgtcccagaaactgtacacctcacttcctcccaaatttcactggctatcacttagtcatatagccacacttagctgcaagtgtgtctgggagatataattatttttcacagtggtatatgcccaactacaaatggaggttctgtcattatgagatgagagaaaggcagaaaacatgttgagagatgtgtagcaatctctggactccacggggataaaaaagaattgagagtatcaaaattcaggatcaaaatcaaaattaaagataaaaaatatcaataactatcacctggaataagaacaacgtacagttcagctacacatatacaagtggcagcatcttgtctggaaggaactaatggtctttctacattgtattttagatatgtatttttttttctcccttccacaggattttgagctccttaagggcagagactttgtgtctcctgctcctagtaggcatccaacacgtatctgtcaactgaaagaatgaatatgagtcagtagatacatattagaattctaatatccactggctgggtccttggtgtgtcccatattgttgtttctgtgtccatcattcttttgcagggtatcttctactgggcacagaacctgcctcagaggggcatatgggtaatgaactaccaagaaaggagtagaacccagttcttccaaccctccacccagagtgcttttcacaacctcatgtgtaataagtgcagtaggagatgagaggagggagtgattacttctgtctggtttgatcccagaaggttttttgaagaaagtgtttttgaatgagacattatgaaaacagagcttcttaaaccttttcccccaaggaatccctgggcagatagaagagacagaaatctgacctctgcttagtctgggggtatagactgaaggaacctactcaaaggagaaatttttctcatttttctttacttcacgattcatatatgcaggcattcattctttcattcatgtatctcacagacataacgaggtcctaattaagtgccaggcattgttttacatgagaccacaagaggccctaccctcttgcagcttacattcttgtacagaatagacatcatacgaataagcaacataaatcatcaagataatttctgaccgtggtaagggctatgaccgaaatcaaacagggtagtcagttacagagtgcatatacctctctgtgcctcagttgactcatctgtaaaatggagataataatagaggtctaggctaggcatggtggctcatgcctgtaatcccagcactttgggaggccgaggtgggtggttcacttggggtcaggcattccagaccagcctaaccaacatggtgaaaccccgtctctactaaaaatacaaaaattagccaggcctggtggtgcatacctgtaatcccagctacttgggaggctgaagcaggagaatcgcttgaacccgggaggtggaggttgcagtgaaccgagattatgccattgcactccagcctgggcaataagagcgaaactcagtctcaaataataataataataataataataataataatagtctataattccaaaacccaaaactgaaagctttgtcctaactcagttgattgcaaacataatatgatctgaatgcatttggaggtagatcttgacctgaactgaagttatttattctttttaataaataaatgagttatttattctttttaataaataaatgagtcatttattctttttaataaatgagttattctttttaataaataataaactgagttatttattctttttaataaataataaataactgagttatttattctttttaataaataataaatgagttatttattctttttaataaataataaataactgagttatttattcttttttttttaataattccacttagagtggacaatcctatatgtcactgcagaaattttgtgtgtttgattatggaatgctgccccaggcctcaatagttattacataatttagggtacatgtagcgtattaccttctaaaatttgaaaaattccgaattccaaaacacatgtagcaccaaaggtttcggataagggattgaagacctgtagtatccattattgtgaggattaaatgaatgaatatatggaaaacacttaaaatgatgcctggcatgtggtaagtgctacgtaagttaactactattactattattatcactattcttacatgagaagatatttagataagttggtcagggaaagcctctctgaggatgtgtcacttgaataggcaactaaggggtggtaatgaccgggctgtgggaagaggaggagaaagatgatttcaggaataggaaacagcaagtgccaagactgtggtggttacaaggctggcttgaatgcagaacagaaaacagaccagatggctgatatgtggtaaaggaggggaaagatggctcaaggtcagagaggtaggctgaagtcagaacacccttgatataagcaatggtagagactttggatttcatttaaagtgtaataggaagacattatagttgatctgattcaggtttataaagaacgctctgatgctgttggatgaatgaattatagaggagaagggggagcagggagagcaatttggagtctagcatagtggtccagatgagacctaatgactaattggagttgggaggtggtaatagtcaaagagaaaagtggacaggtgcgagaaaaaagtttagaaataagtggggggcgggggaggttttctgattaatttgcattctaatttataatatgtcactgtgtagaggctaaaaatttcacagtcattgtctcaggtgtgttaaggccagtggcgtgctggaccccacttgaaattggccatggagggaatatttacactatagaaattgacaaatgctacaaatcaagacaacaaatcaggcaaagcttcttgttaaacatttaccatcacaccactggtgaaggtgacttgatttttccacaactaaacttccttcatttcacagcctccattttccctgatcacgaaaacacttaaactaggcacatcctcggaaacgcagtatgaggactgctgtgtcaatcacttcatgtttttaactcaattcagcgatcctcccacttcttcccaggctctcatttaggtacatgggaatgggatgggaagagggacctggttcatgattgtcatttacccaccttggccccctctgaagtacaactccactctctgctttacaatatcactctgggcagcattaccaattgcctcctgatagtgggatctatgaacccattatgtctttggacaaaagcatagccaggggttgggtccagggcctgggatcctataaccgtacaaatcctattatcagggactataaaatcctattatcagggaccatagccatccctctatcttgactcaactcctcctccctgagtagtgaacatttttcctaaatctctgagaaagactggtgctctagaaagatgtaccatatttatttaagggcttcctgtacccactggcatattgccatatattctgaggtatctgagtgctccttttgagaaacatagccttaaaggataagtagaaatctggtgggtgaaaatggtagggaagaggacttctaacggagggacttgcaagtcagggaacttgggtttatcgactagtgaggctagtagaggaattcaatcaggtaagccggacaagtagacagggtacaaattatggaagactttggatgccatgataaaaagcttcagctcatactgtaaaaaataaaataaaataagaaggttgggtgcagtggctcatgactgtaatttcagcactttgggaggctgaggtgggacgatcgcttgagcctgggaaacaatttcaaggagttcacagcaagaaactgactgattaaggtttgggaagcttgatagatagggtagactgggaaagtgagagaggaggctttggagtggaccaaggatagagggatctcagctgatattatgtcagctaaaacctcaaagcaaggaggatgttaagaacaatgaaggaggtcagctggactctcaatgtttttaacgatagggaggaaaagataggggggtgacaagaagaagagacaattttgtacctctaactccaacaaactttagacctgaaaaatcccttctgagccatcttgcattggagaaaaaaaattgcttatttacctccaattagaggaattaagggaagtaggatttttttgtttttcttttgagacagggtcttgctctgtcaccctggctggggtgcagtggtgtgatcacggctcactgcaacctcaaactcttgggcttaagaggtcctcccaactcaacctcccgagtagctgaactacagttgtgtgccaccatgcccagctaattttttattttctgtagagagaggggtctcacgctatgttgcccaggctagtcttgaactctggcctcaagcgatccgcctgccttgtcctcccaaagcgttggtattagaggcatgagccaccacatctggtggaagtaggcatttggtttcttagataacaacatgattggttgattcagtcacttgggaagataaaagcattaactgagctagatccctatggtagagacacaggctggaccactccatgcgtaagtactaaactaaaaccagtgttctggagtagacattgctagaaatcctgaaacttgagagccagtccacggttaaagcattctgtaaggcagagccagtggaaggtaataaggtgatttttaaagctcttctgcacttcccatattcccttttagggcctttctccctagggtcccagtgtctgtcatgctaaacctagatgcacaacaatcatctttatgggtagtttcccatatgtcccagtttgcctgacagactcttggtttatgcctatagtcttggtgtaattattaccagccccacttcattcttgtaagtatactaatggatcagttatacggttcctctgattatgtatcacctaggcagtgccctgactctactactatctcctctccaaatttatgtaatgtaaacccaatgtgtagggaaaatgctcatcctaaaatctccttggaggggataatttgcaagattctttgcaaaaacaatccaagacaagagccagattatggaatgtcagtgccagaatggcaggaatgtatgttttctaatcaaatgccacttactactgggtaaccttgggctaatcagttaatattgctgagcgatgtcttcatttgtaaaacgggaatcttagaatattctgagactcaaatactatgaaagactcatgtaatgtgtaccagggcaggtttagcaggccgacataaattgcactaaagtcttcatgtgttatttttcatgggtgtatccatattctaacatttcttcaccctccaaatttcagactttggcagtgaatctatggctctgcaattttagtgttccatgtaacaacgaataggaaaatgctgcttctaccctctcgaaagctattttgctaaagagctaagatgctaaaagctaaatatgtaactaaatagttgcaaatctcagtaactgacaaatacagtcatggggttggggatgctgtttagacagctgaaaataagacctgaattgtttatttttaaaatgttgcaaaagagaggcagcaaatgggaatttttaattctgattcttggtatgttttagaacaatgatttgttctttcttatactttcaggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaactatttcacaacttctgtaagctttattgtgtttcatagtttccgttcttcttctgtgagaacaaggataatggcattaaaaaatcagcttttggtcattataaattgtcttctattaaaacacatatacacataaaatcacttgaagacaatttaaacatcttctgaaatggatcaagaggaagggaaactgaaaataatgcaactcagaaaccacagagtattttgacatgaggttaagcaccgtggtttgttgtaggaaaataacagcacaccaacagatggtttttatctgaattctttggtaatcttgacatgtcattcttctaactttctgagggccctcagtgcagttttgtaggactggagctgttcacagacggtccccacaaagctctgaacgtggggcttctctgctgactggcctctggttggctccaccccggaaggaactcccagattctccatgaattccgcttccaccatcaagccttggtccaagcccctttcaaccttgacttggccaggaagtgtcctttctcttcagatagatactacaccttagcaagacttggcatttttagaatccaagccaagggaggcacttggcaaggcaaatgttatggatgagaaaaaggcaaaacaagtgtctgcagtttgtagaggagagagaggatgagtctgattgtagccctgaccctgagtcaggatctctcggccccatttgcaggtctacttccagctccatctgtctggacactcttttaggtccagatcatctcttacatgtggccaaggaatatagagtatgcaaggggatgtagcgacctgagagtgtgagtaacttgtgcccatctccaaggaagctgtgatggggatagcaaggacacacactcttcttatttataatgcctttccccctcccatgagatatgctttttatttacttcctccttctcatcctaagtcgggtgaacaagaggaccaggttgcacatcctactacttatttatggcccaattttaacatgggggtggagttgaggttggaattgttcctccgcctctgctgcacatgctcagtaagcaagaacactgttgatgggaaaggcttagtcacagacagtgggagcacatccctccttggagctttgggtcgctgtgctccagaaacagttagttatagcacaccctgctcctggcatctactggaaggtgaagcccttgaccctaagaaacattgggaatgatttgtaccctccaaagtccaatagctatgtcggagggaaacgatcaaagaacatgattgaggagactcaaacagagatgtgcttcagacaacaccaagacagaaaattaatccattttaccaagttaacaatgtactgaaggcgaacaagagaccaacccacctgccaaccaacgctatgaagaaggagggttgatcagtctggctaacatggtgaaaccccatctctactaaatatacaaaattagctgggcgtggtggcacactcctgtaatcccagctactcgggaggctgaggcaggagaatcgcttgaacctgggaggcagaggttgcagtgagctaggatcaagccactgcactccagcctgggtgagagagtgagacttggtctccaaaaaaaaaaaaaaaagaaggagggttaaaaagagaataagtcccaaactcataagatggtgtggaaagggccctggtgacataggggccacccatgccagtgagaatgaaatcacaacagggcagtttcacactgtttcaggtttttattttttcttcttcttctttccctcctttcttcttttgcctccccctccctctcggtttcctttttggctctagacacccacagcaagtgtcaagcaatgtacaagaatgaaaagaagacagccgttgttgcaggtggatgcttctgtttggaaggtgtggttttgtgtgcacttttggttggaaacctatgtctctctcacacacatgtcccccacctgcttcagtgagca

SEQ ID NO: 7 - DNA, 20529 bp, Homo sapiens
atatatatcatatatacatgatgatacacacacacacacacacacacacacacacacatatatatatatacgtatacaagcatgctttacaaggccaattgactggtctacaattggctgacacttggtggcctagaagccagggtatgtgagtctcgcttttctagaaagctgacaaactctccagttccaaggatccttgctcagtcaacggctggaagtcatttttacttcgctgttttttgtttgtttgtttgtttgtttttttagacaaagtctcattctgtcacccaggctagagtgcagtggcactatcatggctcactgcaatctccacctcctgggctcaagcgatcctcccacctcagccacccgagtaactgggactacaggtgcacaccaccatgcctggctaatttttgtatttttttagagacaaggttttgccatgttgcccgggttggtctcaaacccctgagcacaagtgatcctcctgcctcggcctctacaaagtgctggaattacaggtgtgagccactgcactcgatccattcttacttactttctttactttatttccaagcaaatgtttggagggaaaccaagagacttggatgcggccagccgaggcctttgggtttacaatcacaaatgtttttggtttgcccatgaaggcccaggctgcactctctgatgtcacaggaatcacctctcaaaccatgcaccaggtcttgaattcccttagggtgtgatctttagaggtccatctaggtatacccacccaagccattctttgactgctgacaggccttccttcataacaaggtgttccacagtccatttatatatggatgtcatctctgcccaccctgctgccaatttggttttctcccactcctggggtgtaaggcaagatgaaacatatcacatcccgttctaaactttattcttgtggccaggggtcagcaaactttttctgtaaagggccagatggcaaatatcttaggttttacaggccaagaagcaaatttggcatattatgtagctacttatatagtaaaataaaaatttccacaattatgtaattgatgaaactcaaaatgtaataataataatcgaaggcagtttttttgtagtataggtttaataatgagaagaatggaatcatttttggaggtgctaacattctgcttggttggaatttaaagttagtgttctgtatcagcaaatccattgccaatgttcatctaaaaatgttttcacttctgggccggatttcgttcaaaggctgcagtttgctgacctctgctcttggttacaccttttgaggcccttgctctccgagcataaaatggaatccatttatcagactaaatcgggaagattaaattttccagcctcacgaatgctcagccattgactcactcgttcatacaatgaacactcattgagcttatactacatgccaggtgctggaggaggcatggggcgcccaggagaaagatgctcgctttgcggccacagcccagtgggagggagacccatacctaccggtgctgtctcagaaacttgtggaacaaagatgaagcaatgttcatgttattcgcctacatctgtgaattacacaaggaagacgagtttgagaaatccgaagttcagtacaaatttatggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtcaattaatgatttggcaacagctaagaaatcacaagttcccttcttttcatgtaaacttctgtaaaacacacgctacgttctgctgatggtaaatagagccatttcaggaagttagccagtttctcttctcggccacctcctgcatagagggtaccattctgcgctgctgcaagttacggaatgaaaaattagaacaacagaaacatggtaagccacttctatttctttagcaaagctttccaacagaatatggggtttctgacccagaaatctgggttggtggcaaatggtgtgagcctagaaagtaataaatgggcaaataaggataaaaattaaagatcgaaacaactgtaaatgcaggtaaagcggcttgctatgatctttaatttgtgcacacgttagtataaaggaattagagagtaaattttgaaaatcaaatgcagtgatgatcttactaatttggacaggaaaataagaaaatttcaagttagaaattgaactggaaatattacttactggccctaccagagacaatatcctcttccagaacaacagggttggaagagaaggtgagggaaatattcttcctttgctatttctgtagaaaaggacaaactctcttccttcacatacataggtcaattgctagatcctagtgaagcctgagcttaacctactgttggaggcttaaagttcgacattaattgctacttttcttggtcagagttttaaataattaggttggtacaaaaaactgtgattacttttccaccaacctaataacatgctacaatttctgtaattattattttacactgtcaagacatagcaggtggtccgtttttgttattgtcaagaactgtcagactaaaaatgaactttacacttctttttaaatgatacattttctagaaaattcaatgaggtttaagagcaattgaaaagtctgatttcaagagagtctcatccaaaatgtactatatatttttccccaaagtccttggagttaattttgacaacaatttaaagtacacttaagtcttttgaagttaatgggtctgccacccaggttggagtgcagtggcgtgatctcagctcactgcaacctccgcctcccgggttcaagcgattctcctgcctcaacctcccaagtagctgggactacaggtgtgtgccaccacgcctggctaatttttgtatttttagtagagacggggtttctccatgttggccaggctggtctcgaactcctgacctcaggtgatccgcctgtctcagcctcccaaagtgctgggattacaggcatgagccaccgcgcccggcctgaagttaatttttatacccacctaatgttcattatggatcttgaaggtaaattaattctgcactaaaattttacaatgctttacaaaatgactgtaggtggcccatatggaattcggtcaactgggccaatgacacatatgggattgcagttgaaattatccaattcctacttgatatttgtaagctgctgtgatagccagtataattgtactgtaagaatgtggtaaatagccggggcccggtggctcacgcctataatcccagcactttgggaagccgacgtgggcggatcacttgaggtcagtaggtagagaccagcccggtcaacacggcaaaacctcgtctctactaaaaatacaaaaattagccaggtgtggtggtacgcacctgtagtcccagctactcaggaggctgaggcaggagaatcgcttgagcccatgaggtggatgttgcagtgagcaaagatcgcaccattgtactccagcctgggcaacggagtaagactctgtttcaaaacaacaacaacaacaacaacaacagattggtaaatagagtaataataaaatcaaattaaacttgcaaaaaatggccactttgctcccactggtggccaatggaggtcaaggacctggctgacctcctgcctaaaggcagaggttgttagccttcgcaatggactcaaatcagagggggagctttcaaaactcctgctgcccagactgaaccccagatcaatgaaaccaaaatctctggatacagggcttggcatttgtagcttttagagttcctaagtatctctactgtgcagccaaagttaagaatcagtgccttagaacatcaacagttttttggtccttttgttaaaaagcacagtccgtttttttaggtggctagaaatgctccaggaagagctgaaatgtatttaccagccaccttggtttgattttagaaagcaaaatagaagttctaagtatgctttctctgaaaagctgagactgcagataagagtgagggcagttgatggagttcattctcctctttcaatcactgcttctcatcctttcattataataatctaagaatctcagagattatgaaagagaaagcagtcttatggaagaccccagactcacagaatattagggtgtgtttcacagggaaggatgtcattacccacagttagtctttgaaacgcagttggacattatttgtaagtgcatcatagtgtcgcctccaggttccattgaggggaacgtcattccaatgcaacatctctgagttcatctgggttattaaatggggttgagggatttgttatttttaaattagtagccccaatttaggactactcaagaccataggacaagcctgtccaaccctcggcctgcgggctgcatatggccgaggacagctttgaatgcagcccaagacaaattcataaactttctgaaaatattatgcatttgttttttagcccatcagctactgttagtgttagtgtattttatgtgtggcccaagacaattcttcttcttccagtgtggcccagagaagctgaaagattggacacccctgctataagacacagtaatataaatacataacctgtggttctggattggcattagcagatacaggctgtgttgattttgcagaaagttacaaagagctgctagttggtgtgtatgtctaaaatcagtagatttcctgtggttctaaggaatgacaaagaatctggaagttctctgtggtagcctgctcagtgcagaaagggaacgtggaaaatccgccaccagcatttgagtcttggaggttccacatagggctatcaggtctctgctgatcactgaaaccagatcatggccaactagccccttggcttcagccctcccaattcattaactactcaggtaaatctagggtcactttcaactctaccacctaccatctgagtgaccttgaaaacattcatctctctgagcctcaggtcccatgtctgtaaagcaggggcctcatggacttctttgggtttttttgtttttgtttttgtttctgaggattaaacaaatgctccctaccctatttcccagcatccagtaacacagtttttcatatttttgtgtatgttaagtcaggacccatctctttaatgataagtgcacttaatgtggtcatgttttcttttgtcttccaaagctgttagtgaatccattgaatttgggatgggtaaaataaagtatctattattaattgtaaatttcatctaaagtgacaaatcctacctgcataaccatttcttaatttcctttcatcatgtatcagtggtcaacattgttaactgcgaatgaatcagaatccatcaaaaattagaactatttccagtctggcaaaaattcagctctggttgaatccaaacattgtgctgaagcagctaagtaattcaactgaggagattaattacatgttataatcaataggttctcttgacacttcagtgttagggaacatcagcaagacccatcccaggagaccttgaaggaagcctttgaaagggagaatgaaggagtcatctttgcaaaatagctcctgcagcctgggaaaggagactaaaaaggtaaaaagctgttaattccaggaagacagctttacgcccctcccagaccacctgcactgcacactacgtggaatttattttagtctcacatggcagcgtccctacctttgtgcccacacatctggtctccgccctggctgcagccctccccttcaggcgaattctgggtgtgtcctatctgctcattgcaactcccagcgaatgagttttcagcgaaggcagactttctgacctgttcttcaaactgcactggtcttttaaaaacgtgtttggtggccatcagcatccaatttcagaagaaagatttgggtgaggactgagagaggctgttgttgttgtgctgtctgtttccttcagaatctgcagaagaaaattggcaggtcatgtactgtggacctaaccaaaggacaaatgatgtatggaaaatagaaaaactgttgtgaaattgcttcctcattagcaataactgtatttggcagggagaggagaagttgggcacatttttttttcttttttttttcatgattcatacgttttctttaaagaagtgggttttgcttttcactgggtgctctaagacaaccccagtgaaagatctggaccacgaagacccagtcatcctcataagggtgttcattgcagcaagctcaagggcatgccaggcaaaggccttttttctggcagcttgaacttgtctcagcagagggtttcacagaacaactgtcatttacctgttctctgctcttacttgattcgtttcccaggactgctgaaacaaagtaccacaaacttggtggatcaaaacagcagaaatatatcctctcacagttctggaaaccacaagtcagaaaccaatgtgttgttggcagggttggttccttcttaaggggctagagggaaaatctgtttcatgctcctctcccagcttctggtggtagctagcaattcttgatgctctctggcttgccgctgcatctctctagccttcacctctcctcatgtgggtggccttctttcctgtgtgtctatttccaaattccccttttcttataaggggaccagttattggatcagggcccaccttaattcagtagatcccattttaacttgatgacatcagcaaagtccaaataaggttgtattcacaggtaccaggggttagaacttcaagttatctattaggggacacaattcaacctaaaaactccccttttttgattctctattctgccacttctactcaatccaggttcttcacttcatcagctcccaatctaatacttatcttatttctagtaagcatctcttccttatcttaactggtccctggggcctggcccgagccccattataccatcagctgttgacatcaagggtggacttctctttcggcacagaaggcacagggctgtaggcttcagccttctctgctttgctctgccccatctactgttcatccacctgctttccattttgctaaactttgtagaaaattcttgtcagctgttgtctcctcctacactttctttgatcttagaggattctattcttttactatggctttaatcggagcacccgactgttaggttcaaccaacagaagttggttgtgctctctcactctttctttctctctctctctctttctctctatttgcatagtggtattttttttttcctctattttattggcagaattgccatttctctaagttattgtagagttgctgtttctctattttatttgcatatttctcttctgccaggctggattgtttctattgattggttctgctgtaatgagggtgacttctcattagtatccttctcacttcatctgggaccagatgccctttgatatccttttggagccacaacttttggtagtcagaggcatgggtgtggctcaaaggaagaacttggctcagaaggtgcagctcttgctgggcctttggtctctgctctgtcttctgagatcagtggctgctgggacctggggttcccccatgccgggcatggtcacacagcactcctatggacttgagcagagcaccctgcaaagtgagcattagcaatccattccaactctgtgcagtcctgcacggaatatagaaggtggagcaatgacagtctccccaacttctctgcaagcaacctgctcaccatttcttgcccttcccatttatgtacttttcaaaatcaggttatttggaatttgtcgactcatgtttcttacttcagtacttttttgggagggcagcattagaaacctcaaactcttaactaaaaaatgtctttgggaatgttctggccattttcatggcccacaatttgctttaagctgctttagactctcccagaggctattttcatcccgaaagaacagagcagagctcaaaagactccagttttggtctctagcagcccctagaggatttccccctcaattcctctctgccttgtatgaaatagaattggatttgaaatcggatgttgaggccttacctccaggctagtgaggccacacaagatggatcctctggacccgcccaagtgtccacctaaacatgagttaccaactaacaatgttttgtttagcatgcaaagggagtggtctggaatctggccttgccctgacatattctccttgggcctttttaaaaaaataatttgtgttaatctgtagttaaaaattataataaggacctgacaaacactacctcagtcagatgatcaaggtacacataaatagtgaaagtcatgttgatagcatgcacccttcatatgatatggctagaatggccctgcacttctgtgatcttcctcccctagactcatcagctcgatctaatcataacaaaagcatcagataagtccccgcccagggacattctacataaccatttcccttcccagttatatttttctccacaatactttccaccatctaacattctatctttcaaaatgggcaagtattttagcctggtttgttcattgttttatctgcaactcaaatacagttcctgaaataaaatatctgcctaataaatatttaatgaatgaatgaatatagcattgccttatccgtttaattgccacatggtatttcattgtgtgaacataatatcgtttatttacccagactactactcataggcatttagattatttccggtcttttgctattgctaacagcctttgcaatgaacatccttgtatacagacatttgcatatatgagggtgtgtctttaggatctacttctagaattgaaattgccaactccaagtatatgtttccaattgtgatagatattacacattaccctccatcttagaggtggtgttaatttagattcctgccagcaaaatttaagagtgtttgtttccccatatcctcaactgcctaacagaatcagtgaaaaatggtatgacagtgtaatttttgagtgaggttgagtatcttttcctatgctttaagagcaatttatgtttcctttttatgtgaactgtctgttaatatattttttcaatttttctattgggttatttgtcttttcattaatgcatatacctgttacatatttataccaagtatgtattaaatactaacatattgatgaaacagagcaaaaagcctagaaatagatccaaataacagaagagttagtatgtgatacaggaagcctataaaatcagtgagcaaaagaccatccaattaataacgttagggtaaatgggtctccatttagaaaaaaataatgtgggtctacacctcacattttatacctaaacaattccagtgggataagaaaatgaaatcataaaaaattactaggaaaaagatgagaaaattgttcataaaactgaagtgtggaagatcctttatgccttacactgccctgagtgatctcattcatacccatggcttcaattgtcatgaatcccaaattcattcctctgtcagaactctcttctgagcttcagacccacatactcagctgcctactggacacctctacttgaatatcacaaactcaactcaaaagcaaacctgtcaaatttaattactagtagccctaccccaaacaatcttcctgctcagtgaatgacacccatccctccaggtgcacagaccaggaacctagaagtcactctgattgcatccctctccctcacaacctctacctccctttattcatccattgctatgtctctcaaatgtacctcccaaatatctcttgaacgcgttcttttctatctctattgccaccaccctagttcaaactcccatcatctcatgactgaagttctgtgccctcttgccagtgaacactgtagaatcaatctaaacatggtgccaccctgcttaaaaaccttcaaaggctcacatcacttctcagatgaagagattggggagacgttggtaataggacacaaaatttcagttaggcaggaggaaaaagttctattgaagaactctattgtacaatatggtgactatagttaataacaacatattatacacttgaaaatcactaagagagtccattttaagtgttctcatgaccaaaaaatgataagtatatgaggtaatgcatatgtgaattagcttgactgaggcattctacatgtatacatatttcgaaacatcatgttgtacatcataaatgcatacactttttagttgtcaatttaattaatattttttaaacctactctggcctttttttccttttttgagacgggtggtctctgtcccccatgctagagtgcagtgcgcaatcatggctcactgcagcctccacctcccagtctcaggcgattctccagtctcagcctcccaagtagctgggaccacaagcatgagccaccatgccccgctatttgtttttgtattttttgtagagatgggatctcgccacatggcccagtctggtgtccaactcctgagctccagtgatccacctgcctcagcttcccaaactgctgggattacaggcgtgagccactgtgcctggtccactctggtctttactcaagtccctggctttctctcagtctcttaaacttatgtgcttagtaagatgaggactgaaaaatgtccacagaacatagtgacatggagatactgagaacctcaacgacatctccattagccacttcctctgtgccattccagtcctctgggccccactgtggcaagcagtcctaccatggcaaacatgaaagctgatgtgccttgtcttagacccacaccatatctctctgaattcctgtcccagggcttctctggaggtacagcctgggaaactcacgggaatagacacagggcctttgcacatgctgctcccttttcctgaaaaattcctttgacatcttggttgtgccttacacatgcctactcaaccttaggattgcagttcaggtttcactccttttttttttttctttttgagacggagtttcactcttgttgcccaggctggagtgcaatggtgtgatcctggctcaccacaacctctgcctcctgggttcaagtgattctcctgcctcaacctcctgagtagctgggattatagtcatgcaccaccacgcccagctaattttgtatttttagtagagacagtgtttctctatgttggccaggctggtctcgaactcccgacctcaggtgatcggcccgcctcggcctaggttccacttctttatggaaatcttccccagttgccttgactaggccaaagtcccctcttcttaggctcttacagtgtcatgcacttcttttttatcacagtgtaaaccttgtaatgttgtgtttaagtcatatctgttgtacccatgagactgggagccaattcatatattgtgagtgtaatcgaacagacttcccaggccacccactagctaatcaaggcagggatgagtccggaaagtgactttgaaatctagcaatgttggaacttggaaatcacacaggctgagatctgctcaggtgcctgaacaaatatagcattgcctgtggcgtctccctcaaagtgccttgcatgtctgagccccgttgccccttcctttggtgtgcctgtgtctcccggtacagatgtgaagcctggagacctgtggctgcctctgcaggagctccatgttttcaagccataaatcatcttagaattcatagcatctagatatattagttttctattactgcagaacaaatcgctcccaaatgtagaggcttcaaagaatgcccattgattggccttaatttctgtaagttagaatctgggcaggtttgcctgagttctccactccaagtctcataaagccaagctgggctgtcatctggaggctctgagtaaaaatttgtttccaggttcatccagattgtcaggtgatttcagttccttgcagttgttgttcgactcactaccccaccaccaccccgaaaacctcatttccttgctagctgcctgcagagagccactctcagcttccacaggctgcttgcattccttgttgtggggccgctacctcctcaagccagaaatagggcatccagttcttctcatgcatcctacccctctgacttttccttctgccgataaccagaaaaaacgttccgccttcaaacgctcgtatgattagactaagcccatccagataaattcccatatgccatatactataatgtcatcacagcagtaatacccgggacaaaattcatgggggtcatcttaaaattctgcctatcacaccaggtatagtagaggcttgttttagtgcaagttaaacattaagcagcaacatcacgatagtgctgcatttgaaaataactactagcaactgaacatgtctgggagttctgctccactttaatttccatctcaaaaggagctgggttttccttggctgttacaaatgggcaataatgattgagcttaagaataatcaatgtccacataaaaatcttttataacatagtgagagtgtgacatataaaggtgttagttcaccggccctaaattttaggagaatttttaaaaaggcacttatctggtttaatccataataaagacatgagttgggctttagtgaaaaatctaggctggtttctgtgttcagtgaaagaagatttgagagttctcttaattacaacccttgatcaaacctaccacattaatctgtttattgcattgtatggttaccaaaagtgatatattcagccctctatttattaagaaacagttacagaaagtgaggcactctcctgtgttactgagggtgcataaaaatataaagcaccatgtgtcttccctagagaagtttcaaaactagcaagcaaatagctattaatgctaatgtttgtgtgatagggaacatatgagtagtaattattccacaaacaattttttgagtgctgtttacatttgaggcacagttcaggcacgaggatttcaaaaggagattgtgtagcatgatggcttgttaaaaatatgattttggaatcagatttgctcaagtcccagtgctacagcataccatccttcaaaaaggtacttaagtctctgagtttgttttctcatctgcaaaatataaataataagaggacctactgcgtcatgttcttgtgagcattaatgtgggtgatgaaatgtttatgaagcacttagcacaatacctgacattttgtttgttattattatcaacataaagtgcccactttccagtcatgcaagaagaaaacataatatatgtcaccatagaagtatagaacaattgtgggaaataccagtaagagagatatagctgtataaataaggtaaagatgactgcctagaagatctaggatgataccatattagaagttgcatctgaactctccttggggactggccaaagtttcatcaagtgtcatgtcagtaggttggtgctataaatatatagcttgcaaagctatagacttactataaaccatagctgtggtccagcttagactcattatggtggtggagtatcttgattaatggcctctgcagaagcttcccaggtcttctcatcatcataatctcagatagcttcatcttcaacttccttttttttgttgtttttgagacagggtctcactctgtcatccaggatggagtgcagtggcacaatcatggctcactgcagcctcgacctcaggagctcaagccatcctcccacttcagcctcccgagtagttgggactacaggcatgcaccactacgcccggctaattttttcatttttttgtagagtcagggtctccctatgctgcccagtctggtctcaaactcctgggctcaaaccatctttccacctcggcctcccaaaatgttgggattacaggtgtgagccaccacacacagcccatcttcaacttcttttagcaccatgaagctgaacatagtaaaaaagtaaaatcattctggacctaatctgatgcaatttatttaattgttaagtgaatgcacacatcaaaattcatacaagtatggggcagcgctgctaatttatttacaaaacacctggcaaatactgctactctaatactgtgcttccacttttgattttccttaggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggatccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaactgacgttaagtcatgatttcgcgccataataaagatgcaaaggaatgacatttctgtattagttatctattgctatgtaacaaattatcccaaaacttagtggtttaaaacaacacatttgctggcccacagtttttgagggtcaggagtccaggcccagcataactgggtcctctgctcagggtgtctcagaggctgcaatgtaggtgttcaccagagacataggcatcactggggtcacactcatgtggttgttttctggattcaattcctcctgggctattggccaaaggctatactcatgtaagccatgcgagcctctcccacaaggcagcttgcttcatcagagctagcaaaaaagagaggttgctagcaagatgaagtcacaatcttttgtaatcgaatcaaaaaagtgatatctcatcactttggccatattctatttgttagaagtaaaccacaggtcccaccagctccatgggagtgaccacctcagtccagggaaaacagctgaagaccaagatggtgagctctgattgcttcagttggtcatcaactattttcccttgactgctgtcctgggatggcctgctatcttgatgatagattgtgaatatcaggaggcagggatcactgtggaccatcttagcagttgacctaacacatcttcttttcaatatctaagaacttttgccactgtgactaatggtcctaatattaagctgttgtttatatttatcatatatctatggctacatggttatattatgctgtggttgcgttcggttttatttacagttgcttttacaaatatttgctgtaacatttgacttctaaggtttagatgccatttaagaactgagatggatagcttttaaagcatcttttacttcttaccattttttaaaagtatgcagctaaattcgaagcttttggtctatattgttaattgccattgctgtaaatcttaaaatgaatgaataaaaatgtttcattttacaagaggagtgtatgataaatatatcatagagaaattggtctttaatataaaagaaattgccatatacactgaattttttcagaactctttttaaaaaactatttggtagaaatcaaaggggaagcagttttcatgacacttttactttaagatacttattaatagataaattctatcttgattccctactcagaagacataaagtcagaatgcctggctgttggtagcctttgtgcaattcccccaaatgaaacaactttggcaaccctttccacttctactgtccccttggttcctctgcatcagtccatagcatcctctatccagtatgaatcttgagatatctaatgaaatttacctgagaataactagaaattatccaagcataagaaaaggaagttgcttcagaatgaaaagaagataaacctccaatataccatctttcctttttagttaaatcttacagcatgagttaccttttaatatgtgcttctaagaaactgaccaaaataatgtgtcatagtgttatttaatacgcacaaagtggaaagcagtgcaagtttgccaaggacaatttaattttgtcacattgcatgctgttttgtgaccatgaagagtttatacaaagatgtttatgcttgtgcttgttgaggtatagggacaaatatctaaaagcaagatcagatgggtgtggtatctcacacctataatccttggattaaaatctacctcaattgtaggactaccagttgaaccacatgcttcccactgccctcagcaaagggcaccttagttagaggaaaggtagagcctttctatggaggaggaatttgtgaggtttgagttttatcagctacctgggagtcagaccctgatagattctccttcacactccctggaccttttcctgccaagtggaggctctcactcagaggaaatctccattcttttgatgcaggtcattcatactcagatattctgcactgttcaagcaataaaaattgaatgagcacctattatgtacaccagttggcactgtgtcaaaatgtacttgtgcagagaccttggatcattggtgacaggtcttcttctcctctgcatttttctcaagaccaggcctcagtgtagcatgtttccatggagtgaaagaggggaaggaagagtgggctttggaaagtggcagctgtgtcatagcagtcagcctctgtgtatgtgaaggactttccagagcccccccactaaagcctccatgctcctcctgggactgccacagttcttgaaactatccatacagtcttcatgagttatttttaatttttttttcttcttttctctttcctccttttccccttttccccactccctagttagatctttaaaaatgcaattgtaacctttatcttcccttcaccagacactccctacagggcaagcttatgtatacgcttacctaaaagctccagagccagaaatctctcccactcggggactgcctcaagagacagcagtcaatttacaacctaaagcatgcccacaacaaaactctctcccacctggaggatatcttgaggcaatggtcactttacaacctagttctgcctgcaatggcaccagctcaaccacctggtacataagacacaaaagcaagttgcatagacctcaccttctcactcccttccctgcatgccattaatgccaactccccctttaaaagcccctgctttctgccccaaaagcaaagtgatacccttaaagtcaggagcctatacttcttccccctaagctaatttttggaataaaagtcattttattgagaacctccataaactgttggtgggaatataaattagtaaaccatgatggagaacagtttggagtttcctcaaagaactaaaaatcgaattaccatatgacccagcaatcccactgctgggtatacacccaaaagaaaggaagtaattatattgaagagatatctgcactcccatgtttgctgcagc

SEQ ID NO: 8 - DNA, 7270 bp, Homo sapiens
tcagcccatcatctacattaggtatttctcctaatgctattcctcccctagccccccaccccctgacaggccccggtgtgtgtcaatgtgttctcattgttcaactcccagttatgagtgagaacatgtggtgtttggttttctgttcttgtattagtttgctgagaatgatggtttccagattcatccatgtccctgcaaaggacatgaacacattcttttttatggctgcatagtattccatggtgtatatgtgccatattttctttatccagtctatcattgatgggcatttgggttggttccaagtctttgctgttgtgaatagtgctgcaataaacacgtgtgtgcatgtatctttatagtagaatgatttataatcctttggatagatacccagtaatggcattgcaggatcaaatagtatttctagttctagatccttgaggaatcgccacattgtcttccacaattgttgagctaatttacacccccaccaacagtgtaaaagcgttcctatttctctacaccctctccagcacctgttgtctcctgactttttaatgatcaccattctaacaggcatgagatggtatctcattgtggttttgatttgcatttctctaatgaccagtgatcatgagctttttttcatatgtttgttggctgcataaatgtcttcttttgagaagtgcctgttcatatccttcacccactttttgatggggttgtttttttcttgtaaattttttaatgttctttatagattctgggtattagccctttgtcagatggacagattgcaaaaattttctcccattctataggttgcctcaaacagaggaatctttaaaatgtatgtcagaacctgtcattcctggactctaaatcttctgctggtttcttatttcagtcagaggaaaattgccaagttcttataagatccgacctcttctctgattccgtcccctaactccactccaggtcttcctcacattttccaagcacatcaggatctttaaatttgtacttgctgttctctctctctccaaaatactctttccccagacaacaagtgtcttgcttctttggcccctttagatttctgcatgaagatcactatcagggaggccatttttgatcattctataaaaaagaaaatcactccccagtctctctctgtttcccttatcttagttatttttccttcaagacaatatcactgcctgatattggtccccacccaaatctcatcttgaactgtagctcccataattcccacatgttgtgggagggacctggtgggaagtaattgaatcatgggggcgggtctttcccatgctgttctcatgatagtgaataagtctcatgagatctggtggttttataaagaggaggttccctgcacatgctctcttgcctgctgccacataagacatgactttgctcctcattcgccttccaccatgattgtgaggcctccccaggcatgtggaattgtgagtcataacatataaacgtattgttttgtttgcagtctctcttttcttaacttctttctagaatataagctatgtagaaacagaaatctcttctgttcactgctacttccccagtgcctagaaaagtttctggcagaataggtacttaataaatatcttgaataatgaatatcgtaaaatcttagtactccaactacctctgttctacgtctaatccaaccacgtgaagcctggcacatctcccaaagtcctcagaattctatatccttcaatttcctcatctatcaaatgggagtagtagtacttccctcacagagtatggtgataaataaatgagataatatacatgaagcaattagtatgtatcttggcacattgaaatctaacctgaaagctttgattctatgccataacagaattcagcagctgaatatcaagacctttgaattcaacaagaagttaagacatttatagttgtctaacaacagactgaagattgtggcttggtattcactggcaggtttcagacatttagatctttcttttaatgactaacaccatgcctatctgtggagaagctggcaacatgtcacacctggaaattgtttttcaacattaatactattatttggcagtaatccagattgcttttgccaccaacctgaagacatatagaggcagaaggacaggaataattctatttgtttcctgttttgaaacttccatctgtaaggtaagtgttgaaagtcagatattggctccagggactttctatatccacaaatacaaaaattgaggggtaactccttgatatcaagtcaaaggctcacaatgtctggtaataaaacaaattactttcaattttcttgaaatcttcaggctatcaaaaggagatgtgagagagggtattgagtctggcctgacaatgcagttcttaaaccaaaggtccattatgcttctcctctctgagaatcctgacttacctcaacaacggagacatggcacagtagccagcttggagacttctcagccaatgctctgagatcaagtcgaagacccaatatacaggttggaaccttactccaacctcttgatgaatgtagtcagatgttggcattttttttgcaaataaaaatcctacaggatttaacaaaccaaataaaaatctaatattatatacttttttttagggttttgagctcatcttcatcattcatatgaggaaataagtggtaaaatccttggaaatacaatgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaactaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataaaatcccacagtccttgggaagttggggaccacatacactgttgggatgtacattgatacaacctttatgatggcaatttgacaatatttattaaaataaaaaatggttattcccttcatatcagtttctagaaggatttctaagaatgtatcctatagaaacaccttcacaagtttataagggcttatggaaaaaggtgttcatcccaggattgtttataatcatgaaaaatgtggccaggtgcagtggctcactcttgtaatcccagcactatgggaggccaaggtgggtgacccacgaggtcaagagatggagaccatcctggccaacatggtgaaaccctgtctctactaaaaatacaaaaattagctgggcgtgatggtgcacgcctgtagtcccagctacttgggaggctgaggcaggagaatcgcttgaacccgggaggtggcagttgcagtgagctgagatcgagccactgcactccagcctggtgacagagcgagactccatctcaaaaaaaagaaaaaaaaaaaagaaaaaaatggaaaacatcctcatggccacaaaataaggtctaattcaataaattatagtacattaatgtaatataatattacatgccactaaaaagaataaggtagctgtatatttcctggtatggaaaaaacatattaatatgttataaactattaggttggtgcaaaactaattgtggtttttgccattgaaatggcattgaaataaaagtgtaaagaaatctataccagatgtagtaacagtggtttgggtctgggaggttggattacagggagcatttgatttctatgttgtgtatttctataatgtttgaattgtttagaatgaatctgtatttcttttataagtagaaaaaaaataaagatagtttttacagcctacacatcctactcatttggcttgattcttctttctggtctcacaggtcacaggaagaaaagcactcctgaaatataatttttgcaaaattatatttcaaaaatgacaattttgcaaaattatatttcaaaaacaaacatcatgtcacttctctggttagaaaaaaattttgtggcttaaacacatgattcagggagagaatgtcatgctcctttaagatctgacagcaatctccttttatatccttgcatcttctttatttttaatttttagagactagctcttgctctgtcacccaggctggaatgcagtggtgcgatcatagctcactgcagtattgagctcctggcctcaaatgatcctcctgtcttggactcccgaagtgctgggattacaggtgtgagccaccacacccagcccctccttgcatcctatcattgggccctatggagctactggcccttccccagaactttcagtgttctttcatggctccagagcccagatttcacatcatgcctgctgtaatgccttccctacttgggtttgttcaggaaatcttacagttctctcaggacacaatccacatatcgactcttctttgaaatcatcctcactctttcccgtaagcatgatgcttcttgattctcttccacactttggacatatttctatcaccaacctaattatgtacatttttaaagtttcaatttcccactagactatgaactcctcaaaggctaagacagacttacatctgcccttgtgtctgcagcagtccctggttcagaactggtgctcaaagaatgtttatggaatggatgttgggttggctagaggagcttagtgggaactcaactggcttaaggatagatggtggaatttaaaggcatattctgagaagctcaggaagagcaggaataggtaaaactcaggtaagaagacagagaatccagaattgtaggattcctaagtagagctcacgtcatgtgaaattgccaaaatttggttgctctcgacctagaaaagcatctacttttaaaaatctcattccatctgtattagggttctctagagggacagaactaataggatatatatatatatatatatatcacacaatactatatctatatctatatctatatctatatatatatattgc

SEQ ID NO: 9 - DNA, 88648 bp, Homo sapiens
attttgtggcccgctatggcggcggtgttgaggttgggtacgggatgcggggtctttgactgaaggggtaggccaagtggaggtatcagggacgtcgcgcggcacagaagaggaccagcctggacgccggggacgctgtcatgtacggcgcgagcgggggccgcgccaaacccgagaggaaaagcggcgcgaaggaggaggccgggccaggcggtgccggcggtgggggcagccgagtggagctcttggttttcggctatgcctgcaagctgttccgggacgacgagcgggccctggctcaggaacagggacagcacctcatcccctggatgggggaccacaagatcctcatcgacaggtcggttcctctccccacccgtcgatccttcccttccctcacccgcttgatctcgtctgatgttgacttgactgcaaggactgcagagagttttctggagccagcggggatctgggggacaccccctcccctgtccccacctcctcctggtgttctggtggggagggggacggtgaaacctgccctaaggcactggctggaattgcgtgccgcgtccgtctccggagggatcgtctctggtcccgcagcccctctcgacccctcaccctgtcgctgggctgcagttggcgattccgcgcggtgaaagcagccagtgcccagggtcttttcctgagtgcacctgggcctgccgcccggcgatgccatggggtcgtgcgctgcttttctacttgccgcgctctcactgctcggtgtactgggagggtaccctgggaggcgtgcctttattcttccgaaccgccgctcactgagacagtggctagaagtgtctcttggacctgtgagttagccttaacctgttatgcccccagagccctcagtggagcgcccgtactttgccggcatgacgtttgatttcccggtgataatccgacgagtttgacagattgaggtagtgagcaaagttgcccgtcagttggtggccacttgacttcgtgcggaccctggccttgctcttggaagagatagtgttcttagggctggtttcactgtctcttaagactgaagggtggagctgggatatagatgtgttgtttcttttcaaatcaaacctgctttaggtcgtcactcgagggtgtctagcgattatgggcagtgggggcctgggattagggatttctaaaggcgtttgatttgaaaaggataacattacatgatgtaggtggtttgctcccctctttcttcccattttttcaatcccccttcccctcgtctctgggttttggggttgttgtggggtggttttttttcttttttgcctgttcggctacttctggggcccggactgaaaagctaaccatgaccaaccattaaactgtggaatagtctctccacgtgaagaaagcccatcgtttgagaccattaaaactggattcttcatagccctggagcatgactgtagggatgacctctgagctggccagaatggacacattaatgaccaaataggcctttttccatccctgacgtttccttttggaattagagctcgaaaacgagaactggtgaagggagggccgcggaatcagatcatgtctggatctgatggctccgtgctgtgctcaagcgtgctgtgccttcacaccatggtttatattgaatgtgtcgcctgagttgtcaggcttgcttttccaaagtgtcacttgtgttatttatcattaaagtttggtaagcaatgaagtctgagctctttgtacagttttcctatcattctgtacatgatttgagttaggtcttccaaaactggtggggagcaaacgccgcacatgtacatgtataatatttttaataataatctacatttgtaagttaaggaggtttacatcaaaatccaattaattttgaaatttaatgaaatgcagtaacttgactaccttggaattttgggcctttttcctgtaaatgtcttttttggtctacattaatatttttggttcccattacaaaagtcagcattaaaaaaataagcagacttttgtttgtttctctacatttgtttttgaaaccctaaacctgagtgttttaagtaaagttcactaactcattcatttattatctgacagttacacgttgacagcatcctcattgaatcctttatgttaaaagcatagcagaaagtgctcccattacttatttggccaaactactgtttggtccatgcaagagaaacatggaagtgtcttcatgtatgttatttccttgagggtataaagttcagaaggaaatattgataccatatcttctaatagttttgctctgttcccagtgaacctccttaaactgcatgtatatgtcactgttccaatgtatgtgtgtctctctatcacgtaacccagcacttatttcctcagccaagtggctagggggcgagcctagccaagattttacctccaatggacgcaagtttctttggtgaagatctctcctgagagttcgggactagcagaaagaagcgaggaaatttcgaccgtttggttcttacggataggtatttatgtatttggtttgtgtgaatgtaagctatgatatttaacttttcagaaaaaaataataattttttgcaagtggcattgaatggttgaccaaaccataatggtaagaactgccagtgaagtgggtaccatttttgctattaatggattgtttgcctttagttataaatgttatcttactgtggaaaggaatttagagtttgttaagataacttgagtttaaaagtaggtgagatatgactatccaaattaaatataaatctgggaagagtttttatacttgttttaatatttttgtttattttaatcggtaagtatgtgtgtgtatatatatatacatatataaaacatcacacacgcacaccagtctagacgttaatttccttttattgaccagcttgttcacattacagatatgatggacgtggtcacctgcatgacctttctgagtacgatgctgagtattccacgtggaacagagattatcagctgtctgaagaggaggcgcgaatagaggccctgtgtgatgaagagaggtatttagccttgcatacggacttgcttgaggaggaggcaaggcaaggtactgctcaagacaaacttacttcagcaacaaactttttaaaatttttaagtatttaaaaatttactcccattcatttttttatactcactctttctgatattatcttgacagtacccagtggattggaaaaacaggagtctttgcgttctgagaggacctcaggatagtttatatatagagccacaaagaattttcccagcttttgagggcagactgggatttgaaaaaaacaaaaaccaaactctttaactgttcttctttaacagtatcgtataaataaaattgatgttcttgtctttgccgtaacagtctttaatacagttcttaatcccaaaattttctcagcaggaagaaattttccacaaaagacgtgtattcagctgtctgtgggtaaacatgtactgacaaaagtacataatgatagatataaagtgtgaatttttaaaactattttacctcaaaagtaggttgaaaaaagtatgttgtatgctttactgatagctacaactttagaaatatataaagtttttctcagtaattttctatttttgttgataaaattctcatttttattcaagaggaagaatacaagcgattgagtgaagcactagcagaggatgggagctacaatgccgtggggttcacttacggtagcgactattacgacccgtcagagccgacggaggaggaggagccttccaaacagagaggtgagtggggagctgcctggactgctggtgtagggctacacgtgtacgcacaggctgcatgcaccgtggtccagtctgcagaacacatctctggcactcatgatagcaccactatgaccacaggagaaaacgggagtgatattccttcttttggtaaaacgaagttaaaaactagaatgattaatggaggtggaaagtgaatgcgttggattatttatttctcattgattcgggtaacagaattactcattcaggattatttgtttctagattggtaacatgttcattaatatcctcagggattcattcttgaggcagtgaaagaataggtgttaactgggataagttaacatcaccgccctctcactgacctgcttccccatatccctccacaactgagacagtgacacatgcccagtggaaggacacagtgagggagtttctactccccagaaaacagcacagcttcctggtagccttgatgccacctagggcatatacttaccacagtattttaaattaaagatttggaatttatgcttttctggattaacatgggaaactttgaatataaaaaatagtgctgctgaaaaacctgggctcgtgtagtatagacacaaatatcctcaatcacttcactaagcgtcgagagctccactaccacagcgctgcatcatggtcagtcgttaattagcagtaatgctaacatgaacctgacaccttaaagacgggtcagtatattcaggatattctgtttaaaaagaagaagaacattaacttagaaacattcaaatgtttacattacatcaaatggagatttaattgtagagctaatttaatctgttattctgaacttcatcggtttctccttaagtaacactttttatctttttaaatttttttattaaaatacacaataatttaaaaaaagagatggggtctcactgtgttgcccaagctagtctcaaactcctgagctcaagtgatcctactgccttggcctcccaaagcactgggattacaggcatgagccaccacatccagccaagtgacacatcttttaacaagtatgaagcaattatagcactttagtagtaaagcaaaatgatgtttgcccttccatcctgtgactgcactatggttctacccatcggcactctccaagggctgcgatcctaacggaatgataggacgtggggcaaacgcacacaccggctttccttttgccctgtctttagtcctgctccttactttgtgggcacaagaattactgttgcacagctctattttatgagcttttagagaaactttcaagtgtaattgtaattatactgagttaaaggccagttaaggtatttaagactttttgcattgactttcaaacctacccatccctcagaagttacgatgcactagaaatgttctatcaggtctaaaacgtaaacacccatttatttatccagaataagctctccttcctcgggttctggatagttctgattttgttgtcttatctctaagccacacacatgagttcagctttctatctgtggtgtttttatcagaaggaaggaatagatactatagccacttcacaaataaagagttgaaatacagtcagcttattgggtccacatctgtggattcaaccaaccacagatctaaagtattggaaagaaaataacaaagttttattggaacacagcaatgctcatttgtttacatattgtctgttgccactttcgcacttcagcaacagagttgaagaaatgaaacaccatatggcccacaaaaccagaaatatttattaatactgtctggcattttatagagtttgtcagcccctattctagatgatggaccattgtctcggcgtaattattgggctaaatgatgttcagtttgttataattattgaatcttgagaacttcagcatgacttagcttatcatctgagtattagtttgctttccccttaagataaagttctctttagtattttacaatgttacttcttttctttctgtaatcgtgttctcagaacattgccttatatactgattaatttcgttaatggaaattgggcccacataaaacttagagcttgacatttcgtgtttaacttgcattaatataagtgaaacacctaacacacacacacacatacgtgcatattgtaatagaatccagtaccactaacagccccattgagcgtcacattctgttaaaataaaattttttttcctgagccatcaatatgtctacgtatgtcttgattttcaaaattactgtattgtattgtttgttagtattttaaagccttgtgatactagccaaaagcattttgatggtgcctccatctctgatctttactattttcagtcaagtttttatcctttagatgttcataatttttcatcattattctatatccatttttttccctcttttttaggggaataatggggcggggacaggccctcactgctatatatccattttttaaacaaaaggttatttgaatttatttaaatctgagtttgtagtgcaatggttggtttttattttgtgctactaaagctgtttttttgtaaataaaggtatatataagaatagaccaaatctgtttaacccatcaatcccaaaaagctatttcaattaaaatgccttgatttttatgaataacttaacattaaggagaagctatttgcctagacaatgttttaatcatttttttcattttaggaaaatatagtaaaagttgtatttttaaatttactttgttttacttttttgagacagagtctcgctctgtcacccaggctggaatgcagtggtgcggtgtcagctcactgcaacctccacctcccgggttcaagcaattcttgtgctttagcctcccaaatagctgggattacaggcgcccgccaccacaactggctaatttttctatttttaatggagacaggatttcaccatgttggccagactggtctcaaactcctgacctcaagtgatccgcctgcctcggcctcccaaagtgctgggattccaggcgtgagccaccacacccggttacaggtgtgagccactgagcccggcttcatctctgatttttgaaagaacaggggactcaaacaaatggatgggacggtgttaaataactgtaacttaatagggattgtaatcaacttatatctgatcagactggaatacccaagtttttgtataccaggaaacctgcttaaaattcttctttggtttcacggaatgaggtttgacaggagatctttgcaaattattgatcgcttcaagagcctttactgtatatgatagaaacacttattttgatgaagatttaaggtttgtttctttaatgtcatctgtttggaaataagaacctcaatagatcattgaaatccttaaaaatgttacctttttaaagtttgctatgatatttttgtacatttcagtgtgtctttttaaactggtaatcatctgagttactgagatgtacttaggtaccttagaatacagaaagataatgtgtagtacgttgtctaccacatagtagacaagtatttgttaagtgaatggttaatgaatacatagaaatggaaaaataattgattatttgtgaaagaggtagtttgcttgggtggaggaatcttgatagttatgcccaggtggtttacaattcaaagatgaaaatcagttatctaggaattgactaccttatgtagtgtcatgctgtcaggaatccacagaaatagttggagagaaatcttagcgataccaattaaatacatacatacctgaaagagcagcggaggggaaaaggaacatggattgagcacctgctaagtgtggaggattatggtagagatgttcacatgggctatctcacctaagccccacttggccctgggagtggatgtgatgacttcacaaccagtgaggaaacagagaacagcagaatcccggagctaacaagcggcagggcaggagtttaactacagttaacccttgaacaacacaggtttgaattgcgtaggtccgcttgtatgaggatttttttcagccaaacactgatcagattgagggatgtgagacccgcatatatggagggtcagcttctttatatatgtgggttcaaatggaccaaatgcagaattcgagcacgtgtggatattggtgtctgcaagggtcctagaaccagtcccctgcgtataccaagggaataccgtgcagtctgaccctatacacactccttctgctgcaccccacctccccccaccaccccacctcatttggaattttgtaaagtgagtttcacttgcgttgtgggtagaagagaaagctgaagatggcttctaggagtaacaacagaaggtaaagaagcagggacagccagtcacatgcttccatcttgctgcctgctgttagcaggtgccttcctccctgcattgttctgaattttttaattttctttttatgcagaaaaaaatgaggccgaaaatttagaggaaaatgaagagcccttcgttgcccccttaggattgagcgtcccgtctgacgtggagttggtatgtgtcctgcatgagcactagttgtcgtcattattatttatcataattcactcctgcttgtgggaaagctcaataatgattatagctgctttttaggcatataatgctttaaaatggtttgtgagttaatggagaaaaagatcacaccctatttattttccccaaaggaaaagggaagaattatagcaaaagagctagactggagcatcagggacttgaggagttgggtgtgattcagcggccatgtagttgaatccccagattttactagcttagaaaaactaaatcaggatcagtggcgagtgggctgctccccacacatagatgtaaaagcactcaagatcaagacagcgttgatttcagtaacgttgctttgttctggctttttaaagtgtgatttttggggtcacttcacgttacattttcttagcagttttctgtgttgtgataggtccctgtggcatctccaggcccaggccacccttccactgatgaaggggaattccaccctggttttcctcatctgagggctttgcaactggttactgtctgttcagcattgacctttcctgctatttcagttatatcacattaaattaagttataacaggtgttaaagcccaaaccaggatttttcctttttttctgaatattcattgagggatttccccattccagtcactgtgctaagctgttttttacatattatcttatttaatgctaacaaccctataagcgaactactgtttactatccctcttgtgccagtgaagaaactgagacttaggaaagtcaagaatttggctaataaatagcaactgggaacgttggtcttaactatgatgccatcttcagtcaccgtgctttatgggatttttatatgtttactgggaaggttgaaaatctttttgttgtgtgtgtatacttgggaaggactcttaagtgttcgtgcctagcaggaagttttttcttggacattttcgtaactggattgcaagtggcatcgatgcaggcattctcaattcttgtttgtgtcccacatcctgaatcactcactgcatggtagatgccgggaaagctccgcacagagagagcatctctcacctcccactgcgatcactcgctgccactctaattgagttcagcgtgaatttgatggttcttacccttcattaatctgatgaagggcaatataaaaatagcccttttaattcctgcctccaacgctttccttctcttcccttattcatttacatatcctctccctctcttttattctttcaaatatgggtaaaataactttttggattttgcctagtataattactacttgtattggtctgttctcacacagctataaaaaaatacctgagactgggtaatttataaagaagaggtttaattggctcacgattctgcaggctgtacaggaagccgggcagcatctgcttctggggaggcctcaggaaacttatagtcatggcggaaggtgaggagggagcaggcatgtctaagcatgtccagagcaggaggaaggcagggaggtgctacacactttcaaatgaccagatcttacgacagctcattcactgtctatcacgggagcagcaccgaggagatggtgcgaaactattcatgaaggatccaccccatgattcattcacctcccaccaggcactgcctccaacattgggaatgacaattctacaggagatttggctggggacacagatccaaactgatccaaactatattactacatatgtttgtttctccatttctagtattgatcattttgctgtagttaaagctgaaattacccaaagatttgatatcctgagacttgtattaatatattttccatgtattatatatattgtattcctatttgttctgaaatatgtttattatgcatgagagacacattaacatgaagctttaaaaaatcacagttgctccatttttattaaatgctaagtgctccatctctatttaatgctaaaaagtttatatgaagttgactatatggaattttacttgtttttagtgttaaaaattttttaattttttattcaaatttaaatatagaggtacaatggaattgtgttgccttaattcctattaaaatatttaatggctttgtgttctcagccaaaataagcatcactaagctcttgatagtctgccagatcaaacatacttgtcactcattggagagcaaagtaagtcttagtgtgtagcaacttgctgtcttatcattagagtttcttctaatgatattatagaaaggcctcttgaatgttgttttgactttgtggaaactgagtgcttgattgagtctctcatttgcgtctttcatttattttatggcagtgtcagtatttcattctcataattattatgtgttttttggcagtaattcattgtgtaaattatacaccgtggtgtccatgttagtggagaaaatgtagaagacagaagtgtctgcattataagttgttttagtgactaggcctcagaattgttgaattgtggttaagtagactattgctgcttaagggggcaggacatggtttgactcactgacaagagaagattggagtgattgggaaagacagcaggtacttcaggaggttcttggtttttaaactaactgttggtttagaacctaatgatgacaggatccttgaggcttttggatgaagagtaagaagtagttagaaattacagcaccccaggctgggtacagtggctcacacctgtaatcccagctctttgggagactaaggttggaggatcacttgaggccaggagttcaagactagcctgggcaacatagtgagatcctgtctctacaaacaagtaaaaataataggccagtgtggtggcacgtgcctgtagtcccagacctgttagggcgcagttccaagggaaatgtgcttgctcgaacacattttatggaaagtggggaaggattcgatagttgctgttgtgtgcaacgcttattctgttgatgaataataacatagaaccagcctttatgaagcacttactgtgtaccagacagtgtactaagtgcttctctaggcatatctctcagttaatactcaaaataattttacaggccaggtgcagcggctcatgtctgtgatcccagcactttgggaggccgaggtgggagaatcgcttgagcccaggagttcaagaccagcctgggcaacatggtgaaactccgcctctacaaaaaatgaaaaaaattagccaggcgtggtggtacatgcctgtagtctcagctactcaggaggccaaggcgggaggatggtttgagccctggaggtggaggttgtagtgagctaatacggtactgctgcactccagcctagacaacagagccagatcctgtctcaaaaaaataaaaatacaaaaataactctatgaagcaaatacagttgttgccagatgttaaagtttagaaagttaagagtaactgccctaagttacatgtgtgagggtcaggcctggggttctagccaagggaccgactccagagctctgaaccactaaagttaagctttatcgaatttgtgcagattagagcattatttcatcataatttaggtactgtattgtcacagaaggtgggtggggagggaaaaaatgttgatttattcttgaattactgtggagtgactggccttttgttcagattcgtaaggactcttgacgtctaatgagccttaactcttggtccccaatgtgtcttgcaggtattttctccccgcactttgttttctaagtgattgtacgactctctgtgcagaatttaagtatagagtgatatatgtccctctattccttatggcttcagaattttaaagcttattttggaaggcttcccacccacaagagtttgaaaatattttcctatatttactgctggtacttctgtatttgtatttttgcctttgaattttgactcaatctgatacttacttagggctctgggcaaagcaggtatttgattttttttctcccctaaccccgtgaggagagggctggccccactgcccctggggttccttgctaatttcccctcttcatgtcatgccacctccttcctggtcccccgatgggtctgagctcaagccttttccaaagctcttaggaaaccgtgcatttgtgtgggttttctgacctgtagatacctttctcctgtttcccatgcctcctctggagcttaaattcacattaatggaatttccagggaaggggaggaggtgttagcccccttccggggctctgtcggcttccttaaacaaaaggcttccccactggatttaaattagaaaaacagttttccttttcttaggccattgtaacatatgcatatttatatgttgactgtattttttaaatctcattgtgtagtaggagtgatcatgatattcttaaatggaaaatgatttacctaaagtctgcctaataaagtaaagaatggctttttccaaaccagatactttttaatcttattcagggttggtagccaaccttgaaatatgtccgtaagatgctttgtttttttgtaaaattacttacacattgctttttaaccatggtaatagaagtagtatataacaaataggcctacttttaagaaattttggatcaacaagttggtttggtcaataaagaaagcctaaactgggccagtatcatattttcctttaagggtcattactatgaagtgatcattaattattgatgtgtttatggaatattctgttttaacaatgaataaccagagcccctgaaaatccaagtcgtggacatgcatacagtgggcctttaacatggaatttaaattatttggggattaatgaataattgtagacatctatcctttttaagtgtgaaggatgttattagttcaaaaattaaattagagaattaagccatttatattttatgaaggatgaagccctgaattcttaaccatccatttttaatgagaataattgccagatttatttgcaaataatttcctagtgttaatctctttgttgatatgaaaggtattttagatgtgtggctttcagcctttggtctaacaagatccatttgtgggcaggaaatgctaagagcatgaggttcgttgaagtgactccttagcagcacatgaagggatgggggagggctttgatgctgggaagaaactcacctggagtgtcccatctgctctggccagaccacacctaggtggtcttcagcttgggatactgcttttaagtgggaatggcagtgagccaggaactgtgcaggggctggggagcagggctcaggtgcagaagaaaagacatggcgtactcttgtggcttttgttggatgtccagaagggctgttttgtaagagggagcctccaagctgtgaaacctagtccccgctaagaagaaggaaagagcagtgattctggttattgagaaccatggaatgtataccctctccctagaaaagtgcttgtttgtaaaattcacatgcacagaggattcacaaaccccctgaattccatcatagagctgaacttaaatatatagaaaaatgttgacttggtgcacaaagaagtcacctcccatgggtctgtaccatggtggagtggcctgcctcagctggcgagctttctcccctgcaaaatcctgtcaagatttggagatgaggagtccgaacagcctgggctcttccagcttaaagtctcgtatctcttaaaattgacagtaaaaccagagtcatttctatgttttaatgaaatcacgtggccggtggacaagaggacaaatgggtgacgtgaatatgtgtgttttcccgtagccaccaaccgctaaaatgcacgccatcatcgagcgcacggccagcttcgtgtgcaggcagggagcacagtttgagatcatgctgaaggccaagcaggcccggaactcccagtttgactttctgcgcttcgaccactacctcaacccctactataagttcatccagaaagccatgaaagagggacgctacactgtcctggcagaaaacaaaagtgacgagaaaaaaagtaggtcccactgcgtctgttccgtccagactttgggcctgtgttgtgggggcggcaggctgggtggttctgggaaaagtgtgaagatacacattcttacagatgcatggttgaaagccagactcgaatttctagaatgtgtctgaaatcctgcagctaaggcgtgatcgttacccctgctggtgcacctttattaaatctttggttaatattttatagataaatgaaatataactaaatattgatgctgtcagaacataatcatctgggtgggaaatttttgccctcattttgcccacttaacatttcatagagaaaacagttatatatcctctcttggattattcaagtaccacagtgttcagggctgtatagctcaattatacatggccacaaaagtgaaaattttacttggattatctattttaagctattatttttataacagtgtctctattttggagttcttactgccaaagccagttagctgtattttgaataaagatggtattttgacaagtctattcatatatatgtatatatatacacacacacacacatcttccattgaatttttttttttttaattggcgacagagtctcgctcttgtcgcccaggctggagtgcaatggcgtgagcgcgatcttggcttactgcaacctccgcctcccgggttcaagcaattctcatgcctcagcctcccgagtagctgggatcgtgggcacgtgccaccacgcccggctaatttttgtatttttagtagagacaaggtttcaccatgttggccaggctggtctcaaactcctgacctcaggtgatccacccgcctcggcctcccagagtgctgggattacaggcgtgagccactgtgcccggcccttccactgaattctgttctcttcagccaaaataagtttcaaatcagttgtgtaaatcttaatgcagatctcatcttcagttttgttgtagttgtttatttctgttgctattattttgcttttcataaatcagtacaatttttgcccttttttaaaaaaaggaaaaaaaaggcagagagaaagaaagcatacagagccccagaccagctggtgctcgatgctggcaaggagtcaccaaatgggcaaaggtcgcaatcctttttatctggccttcttctggacaacttgggtgactctagggagaaatttctaaaagtgttttttcgacagataaccaagataacttggctgcttctaagtttttgcataattaatttgtactttttttctcaccaaacaccaaaatcttgaaatgtgattttgatttcagaatcaggagtcagctctgacaatgaagatgatgatgatgaagaagatgggaattaccttcatccctctctctttgcctccaagaagtgtaaccgccttgaagagctgatgaaggtttttatctcattgttgaactatatttttatgccaccacaaaacttctgctaatgtaattttggaaaatttgaagcatgtcattcttgtgtgttacagttgtatcttattttatcatcattgaggtgtatttgcatttttgtttttagctgggtgacaaagcatctgcttctttggtttcttacctgtctagcttataaaattcgtgagcatttgctcaggataattttaccattttattacaattttactcctttgagatatttagagttccaagtagagtgttggttaagacttgaaaattgttttgttgtgcgggtgtggtggcttacacatgtaatcctagcactttgggaggctgaggcaggtggatcacctgacatcaggagttcaagaccagctggccagcatggcgaaaccctatcactactaaaaatacaaaaattagccaggcgtggtgggtacacctgtagttccagctactcgggaggctgaggcacgagaattgcttgaacctagtaagtggaggttgcagtgagccaagatcacactactgcacgccagcctgggagacagagtgagactcaaaaagaaaaaaaaattgtttagttgtgatatcatcataggattggattttataggtgatcagaatatatgcatcttcgagtcctatgttaccatcatagattgtttttaaataaatattttcacttctaattctcccctcatctgtgtgaagaaaccactcagcattatcttgtggttaattcacaccactctgccattcgcgacataaaaaacaggagtctattagatttaagcatctggttttcagcagttgtgcattgtgggtgaccttttgtgggaatgattgctgattgattggactggaaaagctattggtgattaaaaatcagaaactcctataaggaaagacagtttcaaattttgcatggggttagacattcacactttaattggtgtcaaactagtcttagttgttcgtctgtccttttcttggtagttattttggaaattgaaaccctgtgttcactcagttcctctgagacagccagctggggcatttggccacaactcgttaggacctccatgggtgcgtgcatgtgtgtgttttttctaaggcatgtacactgagtcctaaaggtgagccttttgcagcagaagagttctgcatggttcagaatattgaatgctaaggctgtgtcttctctgtttccagcccttgaaggtagtggacccagatcatcccctcgcagcacttgttcgtaaggcacaggctgacagttccactcccaccccacacaacgcagacggtgcgcctgtgcagccctcccaggtggagtacacggcagactgtgagtactcactgtgtatgtcctgacctgtgttcagctgcctgtgacagagccagctacagggctctaaaccccaagtgttctgtcctccaagtgtaacaagtatggaagcaggcggcccagagcctgcacatggtcccaagggagagtgccacgaggctgccctttgcttggcccagtgttggcaagatggctgccctactccagcattagctgtgcattccaggaaggaggaggaccggcaaaggtagctggctgcctctgcccttctttcttttttttttttttttgaggcgaggtttcactctgtcacccaggccagagtgcagggatgcaagcatggttcacggcagcctgacctcccaaactcaaacgatcctcccacctcggtctctctagtaggtgggactacaagcacgataacactgggctaattttgattttttgatagggatggggtctcactatgttgcccaggctggtctcgagctcctgggcttaagcagtcctcccgccttggccttcaaaagtgctaggattgcaggtttgagccactgctcctggccctggctctgcccttctttaaatatccacccaagccatagctagtggcttcccttacctctcagtggctagtgtcctgtcacatggtctccccactcagcagaggcaatggggcctccgttaaacacgttgctgccctaaacaaagtcaaatgctggtaagaacagggagaatgggagacacatagtattgtctaacacagttgctttctttaaaaaggttcacagcaggccaggcgtggtggctaatgcctataatcccaacactttgggaggctgaggtgggaggatcacttacgcacaggagtttgagaccagcctggacaacatactaaaaccgcatcttgacaaaaaataaaaaaaaattagctgggcatggtggtgtgcctgtggtctgagctacttgggaggctgaggtgagaggatggcttagccccaggaggttaaggctgcagtgagctgagattgcactgccacactctagcctgggcgatagagcagtaccctgtctcaaaaagaagaaagaaaaagaggcttacagcataagttaacatatgcactgagaaattacatttctttttctcgctgattgcagttcttttatggtattcattaaaggtaagtcttgaaggtccatgcaggagatcatttgaaagtgtttgacgttggttccagcgtcaggtctttctgtaattgttttattcagaggttaaatatggaatgaggaagctttagcagagccgaggaaccacctgctgagtctgcttcccaggcagctctggtaccctgactccatttgtaaagcttatctccttcagttcagccggagatgaattgttaaaacatcagctcctctttatttgggacaagcttttgtaaacatcacagctgtgttctttgcacttcccttttagcactggcacatactaaacgtttttagactttaaaaaactagttactagagtgaactttctgcatgtgtccccccaaaaaccttttaaagctgagaatgtctttaaatgattaaatcaagtcatatcaaatttcactgaatgttcaaatcagaggtcagctctactgctacagaggtgcgtgttcaatagtgtaggcagccagctgtctgaggtgctctgtagatcactcctaacgcccagtcctcactgcatggatttttggatagacggccgcacacctttaagtcttgagccccactcggcagcctgtgaagctcccgccctggagtcatggggcgctgtgctctgcccaggatgcctgcccactgagggaccatccctctgcttcctcctttccttttccaagcctgtcgttgagtttgcttgaaccaaatgcattgtccgtgcacgtccaccagatccctgaagctgctgcaaagcagaggactagaaattcagggcgggctgaccttgattatttgctgtgctaatcactggtggaagaacagccatgtgcagaccccgcaggaccaggcaggatggtggagccggctggtagtggccgttctgtgacacacagcatcccctggtctggtgggaatgtgatctgaactgaggcatgcaggggtggcattgtgagctgtctgggtcagaaggcttggtacattcccaagggttcaccgcagggcggccagagcccacacactttggtttcttcccacctgctgatggctcccgagaccactgataagccgtgacagcctctgcaggaaccctaagcttactctgttcaggcccctgactaccaccaccctgggcactgacggcaccccaccctatcctcccaactgcaagggctctagtagggggtgccctctcctcccctcaatacggtgccgttgttttgaaactcatcgtctcccctcgacacagcaagagtagtggatacacacatgtgagagtaagggtgcctgggggctggtgaaagcatgcgtgtctgctgttagggtctgtgggttttagacatatgctcctgcatcagccataggggtcagagccctcctatgagcctcctcgctgagcacagcactcagggccaccaccacagtcccacccatcttggatctggagggtcagaaggtgggggaggtgtcctcatccagtttccaagaagagccaagagctagaactttggctctaaatcactgtaaaacctagcagaaatcagtataaacctgtactcaggcgctcagccttatgggatgagtggctgtggcgtggcgttacgtcgggtcctccagcaccacgcaagcccgggcagtgcggccattccagaatctgcagaggttccagggcgcctgactcacacgcacctccctgcctgccgtcttcctctgctaccctttgagtaccttgttctgcctgcctcatgcttctgtgtgctgttgaagtttcgtgggtgaaagtccctcatgacctcgtcttcacttcctgggttttcagtgaagttgttgcagaatttggggtcctgtgtggcaggttgttggcagttgcaggtggagacagcagtcattgatctacccaggttggtcatgattagggaactgcctgtaattcatggactgactactatgtggttattggtttgtaatcagtcattgataacagcatttatttacaaatacagttcaaatagagggaacactggtcatagtttttgggttgagttccgtcatgctaaagttcaagataatattgttacgttcattatatgtagtttccaaaagtattaatgcagtgggatctcaactatgcttaaaataataaactggagagagctgtgcaaaaaatactatgaggctcagagctacctctcaaattggcatttggttgattttttttctctgcatacttttttcattttcatgatgtttttgagtatgcattactattttacttttttttttttttttttttttttttgagacggagtctctctgtgttacccaggctggagtgcagtggtgcaatctcggctcactgcagcctccacctcctgggttcaagcgagtctcctgcctcagcctcccaagtagctgggatcacaggcgctcgtcaccacgcctggctaatttttgtatttttagtagagacggggtttcaccatgttggccaggctggtctcaaactcctgacctcaggtgatccgcccacctcagcctcccaaagtgctgggattacaggctgagccaccgcacccagccgtgttgctattatacttaagaaacaaagtaaaatacaaagttcatagaataactgtaacgtttgcaatgccggacagtgagggcaagagcagcccatggcttggcctgagtttgtggcaagcccaagcctgtacagatctcccgaagttccttctcagactgttgtgaggacgtcgctgagttgcttcaagaaaagacctaaactcatgggctctgtctgatgagcctttgtgaatgtagtgtatgaggttggtgggctattttgaaattcctgcttcagccagcacagaggaaggtttgaggggcccctttctgtcttgggcacacccagccctgctcaggagagcttgagaagcaggtctgcggattctgctgcccttggctgctctcggcttgctcctcgtctccgcctaatgtacccagtgtgttcacggaagtgttgtcccatggggtttcatatacagcctgttattcctgtatctctaatgtgtgattttccatgctctgggcatgcatagctttgtttcttaaacagccagctttctacagagaaggcacaaccgtcagaggcattgaagtaattttcagaagagggcttaaattgtgggctttgcacttgggaagtactctagtaggatactagagagaaagctgtctggaaaattactaacattactgataacattttgggagactctcagttggggcaaacctggggccccgtggggtctggaaaagggcggggctagtgtccttggagcacgtcagcttccacagcagccagttacttttcctgaggacagaggagttgcatgtgagggaggaggcgtgatttaaagcatgaagagaatcatgccccacaatgaaaccagagccctgtggcccgcgtttcagaccactgccaaccatggacaccagagacaagacaaaggacattttggccatggacttgaaacgtcagctgtatgagagcgggcgggggatggcgcggtctccctggtacttactgggcaggtgcgtatcgtcaggagctcctcaccctgccctgtgagaactttcgtatgtgtgtctctgccatctcctcctcctcccattcctgacctgttgagccaggggtggattggcaggcctataaggcgcctttcacattgagggtcttaggatttgcagtccagctttgcagggagcggcagtttgtcatttgtaggaggaaatttcacgatcataaagcacggcatgcatcctgagagccaggcagcgacgctgcctgcactgccccaccgctcagagggccacaggagcagggcttcctccttgcctctgagcagtggagccaaggctggaggtgggcgcagctccatgttctcgggggatttcttcactgtgtttcttgggggctcaccgactgcagccgtattcctggagagagaaggaggcctgtcacagcatctgtgacagcccggaaggaaacagcagtccatacagtcccctcaggacaggcacagaggactccaccctggagtcacaggcttggtgaggtgggggacaggcaggggtgggccccgaggtgtgcagagtgtgtgttcaggcttgtcttcctgccgcagcgcagcagccctcccatgcctgggtcctggcacctgcccctccactccccatgcagcttcatcctccagggcgtggtctccagatgacttacctcctagatacagacaagaccccaaacacacacatgggagccctgagcccaccctggggcagggtgacacatgggagcaggtcagtgccctgtgtgtggcttgccaccatcatttgggaactattcttctgtcctaggtgagtgcccaccctgtggcactgagacccaacagctcaggtgacagtgacacctgcagcggaggctggggaagcatcagagcctctgctgtggtggacgccaggtggcccctggcacagagagcgtgttcatcgctggctcctgccgccctcgaggacttgaaggctgacgttgggctgggtgtggctcgtacataggacagggcccacacactggattcacgtttttcctcacaacttagaatagcaaagttacagactttggattcttacgaagacaagaatgaatgtcttggctaaccatgatcttacccaagaggacttaaaatgaatgtgcaggagaaatgagaagaggacttaagatcaaaaagagagtgactaggaggtcagagaggcagccgcccaccccacgcactccctgcttgtaagccggggccgcattgttgtgttaccacacttctgttttagagcctgttacggttttgagttacacagacatgtgtgggcttgtgcatgtttgaatgccctgtggacccggagctctgtgaggcaaaggctgggactgtcttactagccagcgtgctccttgcacctcgatccaaggggccacggggctcccaggaacattcaccgagtaacttcagaaaagtgaagagcagaagttccaaaagcacctggtgcttcctgggagaagtcacctgcacaggtaccttggatccaactgacaggtgagatgaacgagctctccctgcgtgcgcacgtctacgtacgctcgtgtatgctgaggagcaggcattggaacatgacggagctgctgctgctgcagccgcagataccatctcagccggcatggcgcatgggggtggggtggggcagtgagggggggcccgctccgagagacagacaggtcaggccggaagcgactgtccgtgaaggtgacgctcataccgtaaccttagcagcaggctgttgccacacagtcacaaaagtgggaggcagcagcagagcaatgtggccacaggcactcggactccagagagctggcgagaggctttcctggctgaaagcagtgacaagtatctgggtctgggggacaagggaaacttggaaatgacagaaaagcccaaaaatcaagtccccacaacctcccccagcgatgagagccgtcgtagccatgggctgtgcatcatccctacacgcccgcgcctgaagatttatgcgcgctttctgacgaacagcctttgcagttgggctttgttgtggctggatgactctgagccctttgctttgcttctctaggcctcagttttcccatttgtaccattagggtattaatttaaataacagaagcactctatcgtattctgaatgggacaccagttaattctggaacattttggaggtttcccattgtttcctgtgaaccccagagagagtttgagaaacagatgataaagggaagacaagatcgtaaagtgtgatactgccatcgaaggtctcgagcctcatagttggcgctttaagcaaaataggcggttaaaaacaggtctacacatgctgtgtgtggacccaaaccatgaacacatgctgggccccagcccgtctgttgctgttcccttggtcttggcgtcctgtggtcctcacgtgagctgcacgcagcgagcagagccctgacttccagtctggatttctgtaaagtgatgccgggcttatattatttcaaggacttcatggttacttctccctcctggagttgctctatggctttttaaagcagctgactttttatccatcttctcaaagtattcagcttcattttcacagaaatgataattctcatctctcactcaaattttatgtttgcataaattttcatcaaacacataattacagtaagtttaactggaaaaaataagagagactctactgttaaaagcaaaaaggcccaggcttctgaagagacgcgccttctcccctggtgtttgtcatggcaccagccaacacagcaagatggagccaccagtccagccagggagcttctgcagtgtttcaaacaaggcggcaccagcacagaaatccacaggccctgcagtgggaagggatgaatgagtccccaaaaacagatccaaataaaaataaggacggagaaaaggaaacaccaagctaacaggaaggggtgttctaagacacaaagagctatttggaaaacatcacataagagcctcactttcacccagtatcagaatcacttccagatgtaaatgccagttgtggaaattcttaggaaagtaaggatgcttcagcagaatgaagaatcatctattttcagccagcagttatattcaattaattttttaaaaaatgaggggaaaaaaaaagcctgtatgtctaaacagcttttacaatcaaataggaaaaaattctgatagtctagtagaaaaaagatctgcagtgcgaataattcagaggcaaaaatgcccatacggtttttaaagactcaccctcactgcctgctcacaggcatcgtgtccgccgaccgtagaacctgaaatccgtagtaaacacccctccagcgctttggttcagctcagctccagcgaatgttaggatgtgaggcttcgtgttacagtagaaaggagcgcactcataggcatacagaacactgtgcaggtctaagacttaggagaagacggagatttctaggctgttggaatgttattttgtatatgcgagtttgggtagcttaataatagagattaaataaaagaaatgcagacaaaacctacaatggagtgccatttttcagctgtcactcaagcagagaaaaggtgacagacgtttattgtcggtgggggtgtagatttgtgccagcgatgtggtacttgtggagggccagcgggcagtctatcagtctaaaacacacgtgcctccaacccaggatgtctgctctcatttactagtatatgggtgaacatagccacatagatactcatcatagtacagttttagtagtagcaaaagactgaggcgacgcacatcctcggtggaggactaatggagtccctctggtgtgctctcgcggtgggctgcagagctctgggggtgggccagggcccgagcgctgcagggccacccaccggaatggctcttcctgagggtggaggcgaggtgactcaaggggagggcagcacgaggcagtctgttcacgtcagaaggggagggaagtgtctgtagtgcttctgtatcggcacctaaaagggcaggtaagaaggaccagtggccagcacagagaggggaactggaaggacgaggtcgcagacaggagtggctgactcacctgcccgtttgcgctgcttgcattttttcagcgtgactcgtcaccctctcaaaacatgcagattgacgtggtacttattttgagactaaactggtatcctgagttaattccttacaactctgtattttaataatacgtgcttttatcattttgtttcatcagctcagtgtgtttttgtttaggcggaagtggcccccgtaacatctttcccccgtagagacttgcatacccagtactgctgtgtctcgtgggaggctgctgggtcactaagcttttctggctttcatacgctgggtatttaataatcaccttaggatatactcagtcgttctttcttatttacttcctactgatggagattttcctacattttggcagcctgggaagaaaagcatctatttttttccttaaagtccagcagaattttatatatatataaaatatgtgtataaaataatttttctagaagctcattaaatatagatgtgtaaaactaacaatttatttcatttattaattttcctgacaatgaactacttttcatgctctttatttttcattacttgtcttctgctatttagctataaatccttatagaagagtataaataaataaaagtaattatgcaggaggcagtcatagtgaaatgctgcccactgtgatagcctgtgggttttttttaataaaatgccaactcagtttttcttaatattctataaatatctgaagtgaaattaaaccattgcgcatggctactatagatattttcttgcttctatccctgttttttaaatgtgcctttgctgtttatggtatatttttcctgcacatcatgactttgaagttctttacttactttccaaacccatttttaaaaatggttttattgtatgtcaagagaagaaggaagaaagcaaaaatgttacccaggattccaccatccagaaatagccattagtgaacatcattagcaaagtggaaaacactgaggtcatcgtgctaaataaaaaggaaagaaagaagcttgttatctgtggaacacaaagatcatctttatgcacgaatatcaattaaaatgttggatgtgtctctagaaatacttacgttaaagtggaaataaacttaattttacttaaacagaagagcctgcaatctaaaaatgaagtaactgtcgaacttcggatgaaagtttcttttatgcctaaagaattcagttctgaaaaaaggttaggagaacattgagaggttgtcattgtagatattttttaaagctgtgtatttttcctgatttttgttagaattattcaactttttctttgcatttaatatatctcgaacatctttgtattaacagtgcatgtgtatctctctcttctagctgtagcaactacctagtatcctgttgtatagatttattgtgaaacagccctccattaattgataatttgattatttgtgacttccaattatttctacttttccagtgctgtaatgaacattattcttttttttttttttttttttttttgagacagggtctctcgctctgtcacctaggctggagtccagtagcgtgatcttggctcactgcaacctcaagctatcctcccacctcagcctcctgaccagctgggaccacaggcatgtgccaccacacctggctgactttttagataaatttttagagggtcttgctatgttgcccaggctgatgttaaactcctgggctcaagtgatccacccaccttggcctcccaaagtgctgggattacaggcatgagccactgtgccctgccaattatttattacttttaagcactaatgtggtataattctatatgcaagataaaaatcttagaaaataaacagctaggtcaaagagtatgtgcatttgtttaaagtattaccagtgactgagcagttgccttctgaaacattgtgtcaatatgcattgccaccgctaggatatgagtgcttcttagttttgtaaccatttaaaatgatttgaatatgctgatatagaaatatatttaagagtgagtaggacagtcaatatactatatttatgactttggttcctttaaagtatagaaatattatttttatattatgagagtttataaatagtatttgcattctattattccccagttgcttttttttttttttttttttttttttttggatggagtcttgctctgtctcccaggctggagtgcagtggcgcaatctcggctcactgcaagctctgcctcctgggttcacgccattcttctgcctcagcctcccgagcagctgggagtacaggcgcccgccaccatgcccgattaattttttgtatttttagtagacagggtttcactgtactagccaggatggtctcaatctcctgacctcgtgattcacctgccttggcctcccaaagtgctgggattacaggcatgagccactgcgcccagcccccagttgcttacttttagttttatggttgactggattcgttttttccctacagcttctccttttgagttatttattcgattcatcttttcttgatcatttgaatttcataaacaagtacgtttttacaagggctgtagttcatgaattctgcctgtcgaaaaatgcctgcctttgacctttatgtgaacaggacggcttgtctgggtataaaactcttgggtaatgctgtgggcctctcagatctctgggaactagaatctgacactgtcctttccactgcagggtggcccggggtagctggatgggtcatgactacctatttgggtttgttcattggttggtttgctttcattgctcctatgctgctctctgagttttttcacgcttgaaaatctttctcagctgccttttaacataaatagcaatttgcagggaacgatgcaccgagtcgcttgcctaggattcagaagtggaattgaacgttgctgtggaaagggcgaggccagccctgtcttccctacctgccctgggaggtggatgctctttctgacccaacacatctgaggaagtcttgctttacccttaacttttattaacttaattgcccatttttcatggactatattgtatacctttcagtcagagattcagttctcatttctgggaagttcttttctgttgtgcctcgagcacctttcctgtcccacgcattggtgcctctgcttgaaggacacagtctcttggttcgggtcacctttcttcgagcctgtgctccctgtgtttctctttggcactcagcaggactgtgtccatcttccctgtcagcgacttttttcagccatgtctatttattccttgtagtttaaattccattggttttgcagtggtattgtttggatccttggctggttttctaagctttacagcagggccccacagcctttctatataaaggtgcatgtcttcagccctgcagctgcccagcctctatgcagcccctgctctgtccctggggtgtggaggcagctgcagatggttgtagacccatgagtgtggcagggcttcactaaagcttaattgatggatacgaggcttttcatttctttttttttcttttttttcttttttttttttttttttttttttgagatagagtctcactctgttgcctaagctggagtgcagtggcacgatctcagcttactgcaacctccacctcctgggttcaagcaattctctgcctcagcctctcgagtagctgggattacaggcgtccgacaccacgcttggctaatttttgtatttttagtagagacagggtttcaccatcttggccaggctgggaactcctgacctcgtgatccacctgcctcggcccctcaaagtgctgggattacaggcgcaagccaccgtgcccagcctagggttttaatttcatacaattttgatgtcataaaatattattttgtttgtttgtattttttcaaccatttaaaaatgtaaaaaccaggccagccatggtggctcacacctgtagtcccagcactttgggaggccaaggtgggtggatctcttgagctcaggaggtcaagaccaggctgggcaacatagtgagactgtctctacaaaaaaaattaagaaaattaagtgaccataatggcgcacacctgtggtcccagctgctggggagagtgaggtgtgggggttggcagaggggagggcatcgaggagttccatgctgcagggagcaatggttgtgccactgtactccagcctgggcaacacagcgagactctgtccaaaaaaaaaaaaaaaacagtaaacaccattctgctgatggctgtacaagaacagggagagcgcctgctggaccctgcctcacagcctccccctctgttgcatttggttatgttaccttatttttgtgctttgttgaattcctgtcttcccagattcatctgtggctcagagagctcaaaggttcctcgggtcacatgctcctgtagcctgagatgccattcacatgccatgctacttccctccgctgcttttcctgggggcgtgtgcagggtctcatgccgtctggtgctccttcttccctggtgtgcaagcctgtgtgttcttggtgtgggtggattctccttgatgctctctcaccttctcttagcacctttttcttcccttccaacagccttcttgggaagacctatcctctgctgtcttttgtgagattctaaaaatgtcctagattggatttccttcccccagtgagggaactacagggagagacgttcttgagtatcacagcatatgtgtcaggcagggccccaggtccacaagccccgttctcctcactgtcaggatccccacggcaggtcattggcatttccacctgcttctttccatggtggggcccaggtctcacttcagccacttgctctctttacccacaactctctggaacctatttttatgtaagaagtcttcaaaacctcagtacagcattaaaaattgaaagctttttactttgagggtcactgatgaaaatggtaagttatgtttagagacaggcttttttttttctagaggaaagttttatttgccagaaagaggtgacttttaagcacagtgggctaaaattccaaatagctggttaaatgcccaaaacggattcattttggtagtttcccagtttgacaaatgagtaatcttgcatcactacagaaatcattcaggtttccctaatccaatttggtgatgtcaaaacaagtcttctcttgttgggggacttttttttttttttaagatactaggtcgtcgggaggttacaacaaaatacagtgtggttgtgatggactgcatgttaagtgattttattgtaagtcttggcatataagaacccattaacagatcattggaaaccattctgtgttgtgatatggatagcctcatggtttatattagtctgttttcacactgctgataaagacatacccgagactgaggaggagaagaagaggtttaatggacttacagttccacatggctggggaggcctcacaatcatgacagaaggcaaggaggagcaagtcatgtcttacatggatggcagctggcaaagagtttgtgcagagagactcctgtttttgagactatcagatctcataagactcattcactattataagaataatgcgggaaagacccgcccccataattcagtcacctcccaccaggttcctcccacaacatgtgggaatagtggtagttataattcaagatgagatttgggtggggacatggccaaaccatatcatcccctctcacccctcccaagtctcacatcctcacatttcaaaaccagtcatgctttcccagcagtcccccaaagtcttaactcatttcagcattaactcaaagtccacagtccaacatctcatctgagacaaggcaagttccttctgcctaccagcctataaaatcaaaagcaagttagttgttttctaaatataatgggggtacaggcattgggtaaatacaaccgtccatatgagagaaattggccaaaacagaggggctgcacaggccctgtacaagtccaaaatctagcaaggcagtcaaatcataaagctccaaaatgacctttgactccatgtctcgcatccaggtcacgctgatgcaagaggtgtgttcccatggtcttgggcagctccgcgcctgtggctctgcagggtacaacctccctcccggctgctttcacaggctggtgttgagtgtggcttttccaggagcacggtgcaagctgttggtggatctaccattctggggtctggaggatggtggccctcttctcacagctgcactaggcagtaccccagtagggactctctgtgggggctccgacctcacatttcccctccacactgccctagcaaaggttctcgatgagggccctgcccctgccacaaacttctgcctgggcatccaggcatttccatacatcctgtgaaatctagatggaggttcccaaacctcagttcttgacttctgggcacttgcaggctcaacaccacatggaagctgccaaggcttagggcttccaccctctgaagccacagcctaagctgtaccttggccccttttagtcatggctggagcagctgggacacagggtaccaagtccctaggctgcacacggcacagggaccctgggcccagaccacgaaaccgttttttcttcctaggcctccaggcctgtgatgggaggggctgccatgaagacctctgacatgttctagagacattttctgcattgtcttggggattcacattcggctcctggttacttatgcaaatttctgcagccagcctgaatttctcctcagaaaatgagatattcttttctattgtcagactgcaaattttccagacctttatgctgtgtttccttataaaactgaatgcctttaacagcacccaagtcacctctcaaatgcattgctgcttagaaatttctttcaccagataccctaaatcatctctctcaagtttaaagttccacagatctctagggcaggggcagaatgccaccagtgtttttgccaaaacataagaagtcacctttgcctcagttcccaacaagttcctcatctccatctaagaccacctcagcctggaccttattgttcctgtcactatcagcattttgggcaaagccattcagcaaatctctgggaagttccaaactttccctaattttcctgtcttcttttgagccctccaaactgttctaacctctgcctgttacccagttccaaagtcacttccacattctggggttatcttttcagcagtaccccaattctggtaccaaattactggattagtccattttcacactgctgataaagacatacctgagactggagagaaaaagaggtttaatggacttaacagttccacatggctggggaggcctcacaatcatggcagaaggcaaggaggagcaaagtcatgtcttacatggatggcagcaggcaaagagaggctgtacagagaagctcctgtttttgaaactatcagatcttgtgggactcattcattaacatgagaacagcgcaggaaagacccacccccataattctgtcacctcctaccaggctcctcccacaacacatgggaattgtgctagttactaatcaagatgagatgtgggtggggacacagccaaaccataccagttttatctcagttggaaaatacttggacacaatgtgtgatgagccaaataataaatgcttttaagtatttgggaggatgggaaggaagatcatattttcttaaaaactttgggcttacatcttaaggagtttttggtttgttttaccatttttattcttgcaatatgagatttatgttatagagagctagtagataaccaccctgcctaaaacgaacaattgccagaagggatcttttaggaattcttgaaaattatctgagttcaggagatgaagtcagaagtcatgagaatggagataattgagtggaaaagagaaacttgcagaaggagaaagagtttctccgccctgatttctctcattcacttctagaggaccttgaaggtttctaacatcccctgggtgtattaggcactttcctcattcttgagaatgcagaattcagtaataaaaacaattattcttgaatcgtgttgtcagtgcctgacatttacatgcatagaatgtggacctctcctggggtgcaggtcttcactgtgaataaggcagcactctaactataggcagagtaagattctcaaatcaggcaggctggcacagtctgaaggacctaaaaatacctgtttcagggatctgcatcttcagatggtaatgaaacttttagtaaggctttttttttttttggcaaaaaaaaaaaggtagtattgtagaattttacattaaatagtggaattgccatgaaaacaatttattctgacattgatccagcagccgaataagcctgcagggaatggcgactcttggcagcgggtcaggctgtgggcttcagagtgggccgcttcctggcttaccagccctgctagggtaaatctgctctcagcggcttcctcctagaatccagcttgaaaattaaatggaaataaacaaacatcggttatggtctgggaaatttgctacatattgcatgttctgtatgacacactaatacatgtacatgcgtagtattacagatgactgcatatattggtgaaccattagcctgaattggagaggagatctcaggtgagtattgggaacatgcacatcatctttgcagtgcagcccaccttgtatctctgagaagtcagtgtgcatgtggagaaagaatggaagggaatgcaggcaagttaagatcacccttgagaggtggttctcagatggagctgtgccgccttcctagctgaggacctacagcgttgttgcagtgtagactcatgtaatggtgccatcttttaagcaagtcttgacttttgatgcctcatttgctgctgctagacccaggcggagcaagcttctctggcatgtgggtcgtttgtttgcagtgtgcatttggtgaaattgacagctgggtttccctgtcccccgtccccgcctggaacatcactgttctgagcctgtagccagtgcttttctgtgacttctctttctttcctgtgttcattcctgttcttgttgcttgtatgtttacttctgtattttgctggagcacatcctccagtagtttcccaagaaagggtacataggaacacaaagttttttaaattcttggatatctgaaaatgccttaattttgccttcccatttgacaggtagtttggaagcacctagaattgcggggtgggtatgactttccctaagaatgtgggtgctggatgccgccatctgcaggagcctttgctgccatggagaagctcatgctggccgggcatgctggctcacgcctataatcccagcactttgggaggccaaggcgggcagatcatgaggtcaggagatcgagaccatcctggctaacacggtgaaacccggtctctactaaaaatacaaaaaattagccgggtgtggtggcgggtgcctgtagtcccagctactcgggaggctgaagcaggagaatggcatcaacccgggaggcagagcttgcagtgagccgagatcgcaccactgcactccagcctgggcaacagagcgagactctgtctcaaaaaaaaaaaaaaaagctcatgccatgctcgttaccattctctcctgtgtaacttgtacaggtgttgagcgatttgcatcatgctctgccatgccaggaacacagtggacaaacctccttctgccatggagcttatgttctggagggcagagccagacagtgacagtggacatgtgactaagagcgatggagaaagtggccatgacaaggggaccagggttctggggaggccagcagtgctggcatcatggggacagggaggcctcctgtgaccagagaccagaggaagtgcaggtgagcccagcaattaccatgtgcaggatgggggatgggacagcaatgggacatgaggtaggagaagcaggcaggtgggtttgcaggagggcttcccagacaagggacttagcttgaccctggttgagagaggtcgccatgggagggattcaggcagaaactgtggaggcaagggtggaaaacccggggcaggcagcagccaagaggctgtacacatggagggcaggaggtgctgcagctggagggcaggactcagagctgaatcatcgggcgtcagccttggggtctgccagatgaactggatggatgaggggtgtggccacctcctgcattgggggactacagaggagaggcatggggagaaatcaggggctctgttctggacacattcggcttgaaatatgtacgagacatcccagtgggaatgttgagtaggtggttaatgcacaagttcaagttcagctcagggctggagaagtgaattttgcagccatcaagtataaatagaattcaaagccactgaacttagaagagttcctgtcaacaggatttagatccaggaaaagagaccgagaggcatggccgctgccgaggaagagcctggggctgtgggagcagcgaggccaccatctgactctggatgcctggagagccgggagacaggaaggctggcttgttcctgcctctcagatgtgctcagctagttacatttgcctggctaaaacacaggggccatctctttaacatttcttattaaaataggtgtgtgttttcagaatatctatacttatctccatagaactcttaactattttaattctttttttttttttttgagacagtctggctctgatctcagctcgctgcagcctccacctcccgggttcaagtgattctcctgccacagcctcccaagtagctaatatttttttgtattttttagtagagacggagtttcaccatgttggccaggctggtctggaactcctggcctcaagtgatcccccgaccttggcctcccaaagtgctgggatgacaggtgtaagccaccctggccagcctattttcattcttaatatacacattgttcatcctccctgacttagctcttccagaaaggtggttgctcaccaatctcctctctaagaaccttctcagcacaggagttctgttctgtgtgttaaattcacacgagattaagatcatgcagagatacgagagaactggctctgatttttgcaagaagccagttgaatagagggccttgggagataattaggcagatttctctgacctatgttaagtagctctgcacgtttcagaggaggcagtattggagaaggacttacaaatgtgcttcctgcttttaagcagcttggttctcgtcatacaactatacttgcctttagggactgtgtaggtacctattggaatttctttcttggatttatttggagtaggctttcgtagtactcatagcgtttattagagtaacattacgtcagcatttaacttagtttaaaacgtagtcccctttgggaaattcaatataaaatcctaagaacagcaacaaacctaacaagatatatgtggtcccagcttactgagggttcaactcgatgatggtgcacatgcaatttgcattcagtagaacatcagtaaaatgcttgagatactaaaaactttattataaaataggcttcgtgttagatgatgtcatccagccgtcagctactgtaggtgccctgagcacatttaaggcaggtgaggctgtgccatggtgttcggtgggttataggtggattctgtgcattttccacttcacggtgttgtcagtgtatggtggggttgtcgggaagtagcctcgctataagcccaggagaatcccggcatgtcgtggcagcctgaggacagcaggagccccttggcacactgtgccctcccccgttcatgactagtaatggcacagttattgtaaagctgatgtggcttttgccagcccagacttcagtttgtagactacagcccagcttgtagattttatttctgttgtcaccctgtactagtccagaaattcttaaaatttagtgttcacgagaattgctgtgtacaacatacaaggggctgtatacaaaatccctgtgtcctatagttggtagtcagtttaaagggcttcagtccagttaaagggttctgtgagctgtatggtgccaccattgtgtgcggcacgtgtcaagcagcttcatggtcactgcaggatattttagcactgaggcattttagaagcagtccaggccgtgccaccagctggcatgaactcactcattaaacacttactgagggcctgctcatgccaggagctgtgtgagcggctaaggttttgtggtcactatttggagatatggagtccttgggaacatagctgcacaccagtcctagtggcgcaggagttgccatagggcgttgtttacagggtccccacgcgagcccagagcaaaggcctcctgagtctgccaaggaggcagaggcttcccgaaagaggtggcactggagataagctgaataggggcctcatggcaggcagagaccctgtgaggccgtgcaaaggacagagacctggggaacagaagccagggcaaggggtgggctgggcaagcggcagaagcccttcgggaggctggccctgggctgctccagatgacttgtgcccgtcctgcctcccaccagggccacagtgtctggggaaggatggatctgacgtcctcccttagatcttcacatccctgacaccctatgaagtgagaatctgggagaagcaacccaggaacggtgtagcggaattcatgaaccactgtggtgttggttcccgggctgcctccgagcatggcagtgccataggacacgtcccacattctctgtcggcagacagagaagtgttttcatctcatcaagcaacacatactttatttctcttggagtccttttgagagacaggatgattttcaaatttgattaaaaccttggagagaatcacaggtgtgtgtggggaagaggtgacagcagcagtggctagcagcagaccgcctcacagaggctgcgcgtgtctcggcttcacagctctcctctgtgagaatctcctgggtctgggtcaagggtgtgcccagagcattgtcagcctgagtggtttttagcgtggagcctctgaagcaagttgtggacctaggctaggatgtccctggagtgttttcagatttgggcatttgtttcatttttacacctacaggcagcctttttttctttttgtgagctcagctcaggggctcactccatcacccaagctggagcacaatggtgccatcaacacctcactgcagccccaaactcctgggctcaagccatcctcctgccacagcctcccaagtagctgggactgtagatgtgtaccatgcccagctagtttattttattttattttttggagatggggtctgatggtattgcccaggcctgaagcatcctcccacctcagcctaccaaagtgctgggattataggtgtgacccatggcacccagcctaaatttttcaaattagctgacatttttgacatttgtagtggatgagtctctgagcagtctgccattttgccggcactgctattttttttaacacttcgtttttatttaacaagatggaaggctcaggaaggtcatatagactaacagtctgcgtgttctttaaaggaatggcgctcagctttgaaaacagtttcttcatctctgttgtgttccagtgtgattgcactttacacagttacataaagaatgcaggtatcaggttggagctgcataatatgtactactagttgaaataattataaaccgttttgttttgtttgtttttgtgaattcagatcccgtcctttgtggccccagtttaaaacatgtttggacactttttaggggtgagactgactgtccagagcaggacatggggtttccgtccttcctgctgaggtgggaggctggagacctgacagtagccagtcggtagtggggtcagttccgcctggccctccccagagctaagcacacactgggctgcactctctcccctggagtgctggcttcgccctggctgagaggaagcatccatacatagtagcctgatggctccagcagggagtgggtggaagcagcagctccccccttccagggatgacgttgtctcttacagaagcacatgcttatattcggattcctgattttgataggaagcctatgttggaccatcaggtcagttcgttggtccagcacatactctgctcaatgcagaggctgcagagacagtgaagacaggacctgccgctgcaggagcctcagagacggtgcctgccctgctgtcagcctcccattgacatccaagggtctcatcccctgctcccggccttttctcagaaatgttgctcagatatatctgtgttgacgataatgtggagcacatcgaacccacttatcttattttgaaaatttggagtattactgtttctgtgtcatggtggttggtgtgtaacatggagctagagaacaacggtttaggagttcatcatgtataattaatttaaataagtcattagcagctggggaatatgcctacagcacataggaattatgctgcctcgccaatctaagatggaaaggtcaagatagtctaagttgtacttctgaaatttttctctgcatagcatacattactggaaaccatagttaagcttttactgtttttcaatgttattgttttaaggtgaattgattgaaagtgaagataaaagttcttaattcgaaaaatatttttgccatctcctaataaagaggaaattaaatctctgtgtagtcagaactacttgcttatctacaacaggactggaaattaaatttcgtaattaatcattgaatcttctgtgattcgtggttctgaacatttaaccccaaaaaggataaatgtacaggatttttaattgttaagacagcgtgcctctaccctacagatacctgcttgtgtgcacagcataggtggcaagacggcatacatcactgtctgtgatggaaaggtccagacacagcctcagtgcccctgggaacttttatttactgaataaattcctgcacagcctgtgttgctggggccgggggctgacgccagggttgccaggagcagctgccttactgaggggatggtttccggattaacgtgtgaatggagggagcagcgtgcctggggaatgaaagcaggtgtcagcgcggggagctagccaaaggcatttcctcacatgtgcatttaggagcataggtggccttcgtgggccgtgtgagcaaagggatgactggttgccgctagagaggagactgttcccaacctgcacattttgaagttaaggaggacattaatttgtctagagagtattcatatctggtgcctttgaatgtcctcatgccattcgctttccatctgtctttggatgcgtgttgtggctttgcctggttcttttaaattgcatattgtgcagacaactttttgtatcagaaaaatctagaaaacagcatggttggaagtgagcagaggcaaggctgcatcttgccgggggaagggctcttgtggctgcattgtggactcatggaccagcctgtggccggccatgctcactccggggcaatgtgtctccacagcgaccgtggcagccatgtattacagctactacatgctaccggacggcacttactgcctggcgccgccccctcccggaatcgacgtgactacttactacagcacccttcctgctggcgtgaccgtgtctaactcccctggagtgacgaccaccgccccaccacctcctgggaccacaccactaccgcccccaaccacagcagagactagcagcggggccacctccacaaccaccaccacaaggtaggtgcagcgtccaccgctgcctgctgtgtgagtcactcagcactgcagtcactggggccgtctgtgtctccatggggggcttgtaatctagatcatatacaggggtccccattgtctgagtagttattattccaaatccccaagttacaaagttgacaggaaaacagaaatggttgtagcacaaactttttagcattgaagttaaaccacttataaagttgaattcatttcacgtcgcacgctggccccagatctccagcatctgttcttgcgctttgtgtcagagtctcagttgagctgtgctaggcaaaatcagtatgcagtgaagctgcagttgtttgcaaaacatgcaggttcataaagttgacgcaggtgatgttggggtgcttcatgagtctctcccaagctgttggccaccaggggaccctggcagctactttagttaacctgtgaagccatcggcagagccctagcttctccagcagcgagggcccccagtgttcaggggacgagtatgagacaggcgctttaccagtgggcctggaatgccctgccttgaaaggagactcctgggaaatggaatgaaacacgcgagtttctgtgaaaacgactctttctggtcatgctgagcaagtcagacaggaaatgaaggaggttgaaccatgcttgccgacttgttttcaatataacaacaacaacaacaaactgcttattctttgttatttctaagaattagcttgtgattggggggaaatgttaattagtaggaaaaatgcaccttttatcactaaaatccccatttttcactcttgacaacaatcctgtctagttgactttagtttctgtcgtgtgcatcaccttcaacaagagcctcccctaacacactgtttataactcacatgtctctccgggcatctgaggcggtgaggacccccgagcagccaggactgagcttggcgagcccctgaagcccaggggtctcacagactcttctcctgcagtgcacttgcccccgtggccgccatcatccccccgccccccgacgtccagcccgtgattgacaagctggccgagtatgtcgccaggaacggcctgaagttcgagaccagtgttcgtgccaagaatgatcaaaggtcagaagaagaattttatatgttaggtatatggcatttgggggtttcgtttagcctttttttaaaaaaatgtaggtacagaattaatttttttatatatttttaagccttttcttggctcaaatgtcttttttttttttttttttttttttgagatggagtcttgctctgtcacccaggctggagtgcagtggcgcgatcttgattgactgtaacctctacctcctgggctcaagcaattctcctgtctcagcctcctgagtagctgggactacaggcgcgcaccaccacgcctggttaatttttgtattttggtagagacagggtttcactgtgttggccaggctggtctcaaactcctgacctcaagtgatccacttgcctcagcctcccaaagtgctaggattacagatgtgagccaccacgcccaaccaatgtctttagataaatacattttttaattggcttgttaaattgcttagacttgggtggtgtttttaaattatgttacctgttttttgtttcattttttaagtaggaattttgaagctacctaaaataaaagcctataattcatggttttcaagaatctgccttaaaaatctagacacaaacccttctttttaaaaaccaagcaatgtcccacgcctcagtactaataaaacgtaaagatatgttgtcacatttgcagcgtgacctgtgtaaccccgggcaagcgatttcgaccccctgtgtgcagtctccctcgtctataagatgagtagctaaaacagtaaccaccttgtgggattgttgagatcagtaaagagctaggagaacagggcctgttgttacttcagtgagcttgtcttggtaaatgacccattttctttctttttctgctcagatttgagttcctgcagccgtggcaccagtataatgcttattatgagtttaagaagcagttcttcctccagaaagaagggggcgatagcatgcaggtacgtgtctgaatgcagggaggctgtgaagctcttagaggtggctccgccttccagatcagaagtcgctttctgtttcttctcctacaggtgaaagggctgggtgattcttcacctttttttaatgtgtgtctggcatactccatctttcacgtcccccttagctctggaacctgatctgttgaaagcatctgcccacgttcacagcattgatgattgtttgtccagcacgttctaaacaaacaaaaaaaatcctgttccttcaactgttcgatgttttggccgtctacagttactagctacctttcatgacagccgggtaccttgcttctgttgtgttaacatgtatgaaatatataaaatataagtgggcgcctcatgcctggccagctggtgctgggggtgtcctgcagcacggcctctgcctgtgcctgcacgcccttccccctcaccagatccccagcgtggtgctggcgcacttggaagtgctttttgtcctacagccccctcttctgcctttgctctgctcttctcagttatatagacaccctgacatttttgtaaagccagttttggtgaggagatgacatgggccttacttctcaggagatttcttcagacccttatctccaatagcccacactgaaagaaactgactcctctgtaggtgatggggataatttggtatttttaaagaattctgagtaatcagtgtccaaagaaaagatactgaaaattggttcccaaggcagtattagggcttcaaagagtatagtgttttttcagacaggagaaaatcttccattcctctttgatacattccattgtaagaaaaaacagcagatctggatttggaagtctgttcccagtgctgcttgggcagtaatgtacaattgccgttgtccagtgaaacatataccgtatacatctctctttttttaaaatttctgtataatttcctgctgacagtttatagtgacatttaatctctaggctgtgtctgcaccagaagaggctcccacagactctgctcccgagaagccaagtgatgctggggaggatggcgcgcctgaagacgcagccgaggtgggagcacgggcaggctcaggcgggaagaaggaggcatcgtccagtaagaccgtcccggacgggaagctggtgaaaggtatgctgccacttgcatgttggccttgcacattccaccataagttggcaagcgtaggatcctcggtgacctcagactcagcgccctcacctgcaggctggggtggggttggcggccccctggaggttgctgtggtgaaacctctgccttccatgctgtgtcatgcttgcctcgcgtggcattggaggtaacgtgagtgtgagcagcccttaggtatgtgtctgtttaacagtctgttcagtgtactggacatttgtacagaaagtttcaaataatcctttgtactccctgggacttctgaaactatttatatgcaaactgttgtaccagtgaaattcatttattaatttgtcaaagcagattccttgagaatctctaccaggcaatacttcactcactcgatttcagttactttgttatgttcttggagcaagactttgatgtcacaggacagacaggcatgtaaaaatacaaagtcagtgtaattaaaaagcagacagaagcaaaggccagagcaggcccttagccaggaacctcgtggagcagcagtgggctccccccgcgggagggaggttctgtggagtagaggcgttcagctggtgttgcgagaggaacgggaagctctgaggcaggggtgcagccctaggcaggagccccgtggtgcgagctgcccggccccgtgttgagatgcggtaggtggtcagcagtgacttcgggggtggctggtgaaggagccttggccagcttgccccggtgcaccctgtcggggaggggccagcacatctgacaggctttaggtcagcggaataactttatccagtctggtgactttgtgatgcggttaagccactggagcgacttcagagatttctggtggcattggtggcctggaatggagtgtgacaggtgtggcagtggggtgaggtgtggcagtggggcgaggcgacagctcttgggtcagaaggaaaggcagagtggagacaagagattgaggaagtgggctggggtgataagaggaccgtcctttgcataaagatgccttgttgtatgagaatggtgatcattcagcgaaaccaaatccatgtggatgaaccgctaactaggcaattcactatatgtgtctttgggcctctcattcgagtaggttacctgagcacaagtgatccagctctcacccttcccggccacccgcatactctcactgggataatcaaaggaatgtaataagtagaggaggaaaatggttactgctctagaaacccggggagaggtactgtctataggtcagggtaaggcagcatacctggagcttgcagagaagtacccttgagactcagggcagtgactccaggggagtcgactgtcagccacagaggggcaggcaggaggctggaacaagctgggagcttcccagaggcagcagtacctcatcccttctcacaccccagaacacaaccacagcccgagcctgcctgctgccccagggtttgtgagcccagggaaggcgcctgacccagccagctatggggtgtgcagaggggttgtgagcccagggaaggcacctgacccctgccagctgtggggcccgcagaggagcagccctgcccacaaggctgctgccaaccagcgtgaccttctccacacttccctgattgtcctagaacctggcagatgaaacaacacaccgagaagtttaccgtctacatccacccaagcctgagacacttgaacagagatctactcaatatctgacaaaacccatgctaactctcagttctcaaaagcacaggccagcctctctttgaaaagatgcggagacagaaatgtcattgcgcccacagagattccaaagttcgggagacacagctgagcctccaggcatatgggcatctctgaaacagactcttgcgtaacaggagaaaaatcttttaagtctctaatttgtattctacaaaatggaaaatattataaaactagtgctactggtaatcagacatggaaaagattgcttagaaattgctgtggagtgtggtggctcacgcccataatgccagcacatagggaggccagggcaggcagatcacttgagtccaggaattcgagaccagcctgggcaaatgacaaaaccccgtctctgctaaaaatacaaaaatgtagccgggcatggtggcacatgcctgtagcaccagctcctcacagaggctgtgaagtgggaggatcacttaagccggggagatagaaaccagcctggacaacatcgtgagaccatgtctctacaaaaaattaaattaaattagccaggcatggtggcacccatctgtgctcccagctacttgggaggctaaggtgggaggatcttttgagcccaggagacggaggttgcagtgagccaagaccacgccactgtgttccagcctgcgtgacagaggaagaaaattataggtatctttttaagtacacaagctacaactagaagttaacactagaaaataaagtatatgagaacatatattattttaaatttgaatgaatttgaaaatcagtgaaaaatatcgctttctaagaaaacataagtgctgaaattatttcatgaagaaatcaggaaccaacatagacctatagacctgaaagaattttgaaaataattggttagtgaaatacctctcaacccaggcagtcagccaagacaaattaaaggtcaagttaattcaccctcaagaaatagaatgcctacatcattaaagccactccagagcattaagaaggatggaaaagtatgaagctctcaaagcttggcaatctgtagaccacgatcacttacaagtatagatgttaaaatattaaactgaattcagctatgtatgaaaatagtaggccgggcgcggtggctcacgcttgtaatcccagcactttgggaggccgaggcgggtggatcacgaggtcaggagatcgagaccatcctggctaacacggtgaaaccccgtctctactaaaaataaaaaaaaaattagccgggcgtggtggcgggcgcctgtagtcccagctactcggagaggctgaggcaggagaatggcgtgaacccgggaggcggagcttgcagtgaaccgagactgcgccactgtactccagcctgggtgacagagcgagactccgtctcaaaaaaaaaaaaaagaaaaaagaaaatagtaatagatgatgagcaagtaggattgttccaggaatcccagcatgtcacaaaatgagaagaccttctagtatagtttacatattaacagatgagtggctttctcagatgccgagaaagcagcaacattctctcagatgttgatgatgaaacttctttagtataacactaattaatgttggaaaaacaggctctccattttgaaaaataataagatatcacatatttgacaaccataattccatataaatccagtatttaaatgtaaaacataaaactagaaaagtagaaaaatataaaggtaattttccttttaaaataatcttggagctggaaagaagtgtcttaagaactgaagctgtaaatcatagggaaagattggtaggtctaatgggggcatggcctgaataaagttaaaagacaaatacagaaatggcagtaactgctaatacataaaaataaaggttaacatcctcactatagaaagagcttttataaatcaatacaaaaagacagtcactgacctagagaaatagacaaaggacatgaaaatatggttccaaaaagaaaaatcgatggctagtaaatatattttttcaatgtagcctcattaacaagtttttattttttgcctctcaaattgataaagtttaaaaaaagtaacaatgagacagaggctgtgggcagtaggaatactgtttttgagtgtaagtcgatagaacccagtaatatggactgagttttaaatgaaaatgaccttctactcctagaaacgtgttttaccaagatgtctatacatggatgttcgttgtagggcatgtttgttttttaaaattaaaaaaaaacttggaaacagttatgggaaattgtgaattgacatgtccatactacttattgctgtctgtttaagtggtctgtgttgacacagaaacctgtctacacagtaaagaaaggagttgctggggccagacatggtggctcacgcttgtaatcccagcactttgggaggccgaggggggcagatcacgaagtcgggatttcgagaccagcctggtcaacatggtgaaaccctgtctctactaaaaatccaaaaatcagctaggtgtgatggcgggcacctgtaatcccagctaatcaggaggctgaggcaggagaatcgcttgaaactggaaggtggaagttgcagtgagcctagatcgcgccactgcactccagcctgggcgaaagagcgaaactccgtctcaaagtgaaaatagcaggttatgaaacgagctgtgcccccattttacacacgtgtgccattgtacacttctgcctgggaatccactgattatgttcagatgattttttttccatattggaattgcaattgatctatttgctcatatgttttcaaaatctcccacagaaaatgtatattacttttgaagttagaagttagcaataagagttgttagctaaaaaacagaaacctatttgctatggaagatggcgggttcactcaggggtgcccgatgccatgttagccatgcatctgtccccgcatggtcccgtcctccaccgcctaggagatagtggaccatcagtgcctgaatgcaaatcatagtgagtggtgtgcaggaaagaggttgggcagggcctgtctgaggaggcatcgtgggggccgatccttgaagaatgtgaaagggacaggagaagagcaggcagctctcaggcagaggtgaggggcagtgcaaacgtggagcagtggccccgatcactcagggacgtggcagccttgggaaggaactgggtttattctgaatgcagcgtgagacctactcactgaagctgtaggacatgctttccatgtgctgtgacgtgatctgcaaggaagattctaggcagaagcaacaattttgtgattgaaaaattccacataaagaagcaattcctgattccctgtactgacctgaggtacctggagaaacttagttaatcttttcagcctcggttttcccatctgtaaaatgggaagcctctcagtgtccatcctgtggagctgtaaaggctgagtaagggaggcctgtgggctgtgtgccataggccactcttagagtgagtagctgtggttttggctttgtgtttggtttgcatagatactagctttaaaatgtctacttgacaggccggacaaggtggatcacctgaggtcaggagttcgagaccagcctggccaacatggtgaaacccatctctactaaaaatacaaaaaattagctgggcatggtggcgagcacctgtaatcccagctactcaagaggctgaggcaggagaatcgcttgaacctgggaggcagaagttgcagtgagctgagatcgcgcctttgcacttcagcctgggcgacagagcgagactccgtctccaaaaaaaaaacaaaaaaaaagtctacttgaggctgggcacggtggctcatgcttgtcatcccagcacttcaggaggctcaggcaggaggattgcttgaggccaggaagtcaaggctgcaataagctatgattgcaccactgcactgcagcctgggcaacacagtgaaaccctttctcaaaaaaaataaaataaaatgtaaaatgaaataagcattgctagaaggtgttctggaagctttcatcttaatactcttatttgttgattgcgtatttttctaatttggggagatggtttggaaataattgttattaaatcattttgtgatatattttagtccagccccttgtttgttttgttttgttttttgttttttttgagatagaatctcactctgtcacccaggctggagtagtgcagtggcgtgatctcagcccactgtaacctctgcctctgcctcccgggttcaagtgattcccctgtctcagcctcccaagtagctgcgattacaggcgcccgccaccacacccagctaatttgtgtatttttagtagagacagggtttcaccgtgttggccctgctggtcttgaactcctgatcttgtgatctacctgccttggcctcccaaagtgctgggattacaggcgtaagccactgcacccggccaattttttttttcttatggaaaatctcaaacatgtacgaaaacagaatatcacataatgaccactcacgccccacacacgtgctcatcatctggcttcagctgccggccctgctgctttttattttgatgttataaaatactgctgtcccggtatcagtatctttgtgtgtgccctgtcccctgaggcagttgttgatgaaaagtggttatatttcagagggtctgggctatcacatgcttaactgtctacttcacatcacacgcgggtttggagccataaaatgctaaagcggaaggacctccgccaggggccagccaggtagcccaccctgtgctacagaggtggcatcacaaacataagttgcagcccttccagaagcggccttgtttacccagaacccacttccctctcagtcacctggtttgcggtgcacttagcatccccttcattgtgggtgccttgaatattcttcataaataacaactgatggttttttaaaatactgtatcttattgcaaccagttagctcttgtcaagagccatttatcacagcatctgaaagagaaagggactctgtgttcattgagtggtggggcgggaaagatgattttttctttagggcctccatatttcccttaaatttaagccttctggatattctaagaggagggattgcttctaaacttctgtcacgctgggtttgacattttcttacaggtgtggaaaatggtctaacataatgcctgtcacaaagtaggtaaaaatgtttgctgaataaaggcatggattctgtaatttttgctttgtaagaaaaggctattttttatcatggggaattttttaaagagacctgtttatagtggagtcacatcatatgcctcctgaagcaaatttagatatatgctgagccatgaatttttttttttttttttaaagaaaaatgaggccgggcacagtggctcacgtctataatcccagcactttgggaggccgaggcaggcggatcacgaggtcaggagatcaagaccatcctggctaacatggtgaaaccccatctctactaaaaatagaaaaatttagccaggcatggcagcgggcgcctgtactcccagctactcaggaggctgaggcaggagaatcgcttgaacctaggaggcagaggttgcagtgagccgagatcgtgccactgcactccagcctgggcgacagagcgagactccatctcaaaaagaaaagaaaagaaaaatgacaagaattggccatttaaaattgcaggtgactgccctggcatcagcgagtgtgcccttgccatgaagtccccagtcagtgcggttctcacagcatggttcaggggctcaccccagccccacgccatgcagtgcacatctgcacaggtctgctctgacggcacggcgtcccccaccgtagaccctgcatatgatgtggctccatgctagtcatccccttcccagcagccgatgctcaggtgggtagcagggcctgcaaagatttccactctgtaacatgtatcataattctcacctttcctcaatagcttcctttgctccaataagctttgcaatcaaggccaaagaaaatgatctgcttcccctggaaaaaaatcgtgttaagctagatgatgacagtgatgatgatgaagaaagcaaagaaggccaagaaagttctagtagtgctgcaaacactaacccagcagttgccccaccctgtgtagttgttgaggagaagaagcctcaacttacccaggaggagctagaagcaaagcaaggtttgttgatagcttttaaacttcttgaaagaaaggaaatacacaaatataagatttatctgctaagccaaaaaatctcgaggctgccaactagaatctgaagcctttggaaatcgacctatttgggagttgtgtaacatgtctgaggttttgaaacgttctcttttagaggaatgagctctgctcttcactgagcctcaaatgcagtgccgctggcagtttgttttcgaagaaactgagttggccgtcttagctctaatgcgccacagtggaatgcattaatggcagctcactttgcacttggctggcagccccagggtaaaaggctcagcctgtcttcccagctcaggaaccaaactaggagatgccctcttgtgaggctgcctacccacagaaccattgggcccttgaaggtggtgtgtccccagctggttttccggctgcggctcatcttcatgggccgcagtgtggccaccacacccaccccaacactgctggcagcatggggacagcatgtagtcttcccatcccgactccagaataaattctgctctgcattaaagcagtcaaataatggttgctgcattgtggttgttatctattctaactgattttcttaaattgcttttcctgtatacacacattcagatcaagcaacatttgaaagaggccaattttcaggccaggcgcggtagctcatgcctgtaatcccagcactttgggaggctaaggtgggtggatcacctgaggtcagaagttagaaaccagcctggccaacatggtgagaccccatctctactgaaaaaacaaaattagccgggcgtggtggcacacgcctgtaatcccagctacttgggaggctgaggcaggagaatcgcttgaacccgggaggcagaggttgcagtgagctgagatcacgccattgcactacagcctgggcaacaagagcaaaaactccgtctcaaagaaaaaaaaaaaaagccatttttcaaccacaatccaccatcaagaacttccattgtgctgtggtgttctccctaagcaaacttgtactcatgcctgtacatctgaatctgtccttcctgtgtgtaaactaaccaactgtcggatcatttggaataaaacacttatagagtattcattgcctggtgtgaatattttggatatatgctgagagccactctgaggttttcattattccagctttcgttagtgtagagtctcaccaaccttctaactctgaaagtaaaatgtccaaaaaagggcacgttataaactaattctctcaaaatttgatttgtccaatgtatgtacctattcagaaactttaactaactgcattgtatgacacttttgcaacctgtgaaaattaagatcagataaaatactgtttgctctaaacttctcttttttctttgtttattccttaagcaaagcaaaagctggaagatcgcctcgcagctgctgcccgggaaaagctggcccaggcgtctaaggagtcaaaagagaaacagcttcaagcagaacgtaaaaggaaagcggcgttatttttacagaccctcaaaaatcctctgccggaagcagaagctgggaaaattgaggagagtcctttcagtgtcgaggtatagtaaaatcccacattggtatctgcggggctgtgtgatacatagaggcagggaggatgtgtctccctccagctgccctagtctctggcctgagtgagggatatgagctcccagctcttcctcccgacatggttgagtggcttttactctatagcagtgaatctaagagtttgccagcagtctcccccgtcagtgcacagtcacgccagcagcaaacactgcccgcgatttcaggggagcctctgcttcacggctgcccttatggggctggcaggagggcttggggagtgcctcccatgggtcctgctggggaaatgtggtggacacacttcactgaagccccgcctccgcagcagcaccagtattgcgctcacacgtggggcagaaatccttttgccacggtctgtatcaatgtcagcactttaattaaagagaaaaaggaagagggagttaagagaacagactccaggagtacatggctccttcctcagtggtgtgagcaggaatagggccttacatgggggtcatcacgtggctgccttacaagtctccctgccaaggagggggtgctcagaacagtgcctcagaccagaggccttcagtagacactggctcctgagtgccaaggggattgctcccttgtgtgtccgagaccagaggccttcagtagacactggctcctgtgccaaggggattgctcccatgcgtgtccgagaccagaggccttcagtagacactggctcctgtgccaaggggattgctcccatgcgtgtccgagaccagaggccttcagtagacactggctcctgtgccaaggggattgctcccatgcgtgtccgagaccagaggccttcagtagacactggctcctgtgccaaggggattgctcccatgcgtgtccgagaccagaggccttcaggaaacacatgccttccgcagcagcagcacagcaattaatcataatcagcaaaaactctacttttttttttgtcacatcaatttagaatcttttaagtttaattttagattctttatagtagttatgtctctgaattttattttgtatttaaactacaagaatatgcagaaattctttggggagtttaggagcattttggagacataactcttaaagtaagaaaaataatagagtaggacacatcctttgaggattaaaggagggttgtctttgtatcaataaactgtgacaaaactgggcattttagtagctagtcctgtaattgtaggtgaattaaaagctgacaacatttgaactataatattagaatgggtttacatctacaattagacaatagctaaaaagttgtggttttatgttatttcaagaacacttaaaaatcattttataaaatctttctcaacctaatctctctctttaaaaaaatgaatgaacacaggaacagaaaatcagacaccacatgttctcgcttataagtgggagctaaacattgagcacacatggacacagagaagagaacagcagactcgagggcttcattgagggtggagggagggagggaggagggtgaagatcaaaaacctccctgttagctactatgctcactgcctgggtgatgaaagaaataaaagttggaaagaataaaaaaggtagtaactccgggaattttactttttgaaaagtttcaaaccttcaaaaaattggaaagaatgggaagatgcccccagcaccccaggcgattgcatgcgcgtgctcgctcatctatatgtgcgcacgttgacccacgcgtgctcgccctctctgagagtcgttgcatatgtggtgactgctctgccctgaatactgcagctgcatttcccatgaagggccttctcctgggaaacacagcactgcatgcagattgtccaccgatggtgtccatcacttcctctgcaggccgcagccacgttgctccaagtggccccacgtgtcttttgtagatctttttttcccaaagtacagaatgagcctttcactttaattatattgacgtttctaagagtccagggccattattgaaaactgattttctgcttgaagtcacttcgcttatttttctgtggaaaacaacattctaagctcagacttttcaaatgatgctgaaggctgaatcagctttcttgttttgggagtcagtctgaaatcctctcacatctggcaggaggcctcagaaataataactgacgggcaaggaggggagaattagaagagcagagaagatgagtttgtgtgagaccctgtcgagtccccgagtgccgcagggtgggctcctgccctgagtcccgagtgctctggccacccgctgtagcctcagctcctctgagccatttgacatgccagccccagaaacgaacattttcaggcaaggtgggaacccccagcagccccccgggacgccgtctcacagcctttccacagctcttcagagtcggggctgcctcctggctcctcacttcagccagttatggccgaaggatctgtggtcattccttagctttaataggatttcttggctggacgtggtggctcatacctgtaatccgaacactttgggaggccaaggcgggtggatcgcttgaggccaggagttcgagatcagtctggggtcaacatggtgaaacctcgtctctactaaaaatacaaaaaattagccgagcgtggtggagcatgcctgtaatcccagctactcgggaggctgaggcaggagaatctcttgaacctggcaggcagaggttgcagtgagccaagactgcaccactgtactccagcctgggcgacagagcgagactccttctcaaaaaaaaaaaggatgttctgcagcaataaggggatgaaatacacaacaacaaaaatgatcatgaggacgcttgtagccacacagaaaatgcttctgatgtaataagcaggagaagcacagtataaaatatatccacttctgtggttacagccatgaaaatatgcatgtagcaaggagggaagggaatttaagaaagtaagggacctgttacagtggcgtacgggttctcatgttttgatatcgtttgtgcagcgggtaaaggggttaattgaaagacattcacaggaatgctttaaccagttacattacatgactatacgtgtatgtcgtcataaaatttccagtgaaactcagtcacaagtataatttatcactagcccagtttttcccaatctgctgtagttccgcatcacagcaaccagaattatttccttataaacataagatatgttacagcttaggtctgtgtcctatttatttattttattttatttatttatttatttgtttgttttttgagacagagtcttgctctctcgcccaggctggagtgcagtggcgcagtcttggctcactgcaacctccgcctcccgggttcacaccattctcctgcctcagcctcccgagtagctgggactacaggcacccgccaccacgcccggctaatttttttgtatttttagtagagacggggtttcaccatgttagccaagatggtctcaatctgtcctatttatttttacacgtaccctctcacctctcctgtttgcaggcattggtttttgaatctgtagaacatagaaatgagcgtttaaatcactaggatgctctccctggatatatgtgtgtgtctgtgtatgcagattacagctaccaagccatttcaacaaaaatgtaatggttgtagcagatgatgataaatgtctttaattgcttctgaaacaaaaatacttgtaattaaattggcaattgccataaagaaaattcaaactcgaaaatatttttagcctaaaacaacttctgggacaggttacccttgactttactaagtattctagcatctgctttactcgctgatgttgagacatttgacccagctatgtagttgtgaaattctcggagtccaggaggacttgagacaagaccacattcggccaccgcacgccctgggtgaggaagcctgcgtggctgagggcacgtcggcaccaggaggctcatggcacccccaggtctgtcggggccgtggctagctcgggctggctctgcagggtggcatgaggacactcccttacacaaggcctggcataacatggcaggaattttgctgtcaccttaaagttaactgaaaacagccacagtgcagcttatgtgcctgaaggacagtcacttctctgtctttactttctataaaactgatgtatacatatgatttttaaagttccaatgctagagaaaggtataaaacaaagaggagaggtcctttctttcttgtgtatttttttaattcctgtggaaatggcactttttaaaattcctccaattctctcccttctgtagagtttgagtttttaacataaaggttaccattttaccgtttttaactgtgcagtttatgctggcattaagtatattcacactgtgcaactattaccacccaaccgttcccaggatgtccatcttctaaaaccaaaactctgtagccattaaatagtaactctctgccctcccctcccccagccctggcacccacctgcttgcagcctctgtgaacgggactcctggggaccgcatgtgtgtgggattctgcagtgtctgtgcttctgtgcctggctgacttcacctagcgtggtatcctctgggtccagccatgcagcagccgcattggacccccttcctttttacagttgaatggtgctcggtcgtgtgcatctaccccgtttgtttccgaacactttgggaggccaagtgaatccttgaggggaaaaactcaattcgattcatgtatgtggaggtgtaaaacctcataggggatcataccctacatatttttctgacaacttttttcacttacagtagagatccttctgttatcaatgcttacagtagtgtttggtgtgttacaggcactcggtaggtatttagtgaccgaatgactttaagtagtttacctgtaaatcaccccctctgtgtgtgcccctcccctgctgtgggaaatgtgagctgtgggtctgtctttgtgaacgatgccgctttcacccaatctgtgtcctgtgtcttgtgtactccagtgagtgcacccacgggggaaatcctcaccatggaattccttttttaatttgagatactgctcagttgacctctgaagggaccgtagcagttttatttgcccccgtagggtgtgcgaactatttcttcatgtccctgtcggcattggttaccatcagcctttcggtgggaaagcagcatctgctttaacttacatatttaatggtaagtaacactgagtcattttgctaatgtctcttttttttttttttttttttttgagacggagtctcgctctgtctccaggctggagtgcagtagcgcgatctcggctcaattggctcactgcaacctctgcctcccgggttcaagtgattctcctgcctcagcctcccaagtagctgggactataggcgggcgccaccacgcccaggtaatttttgtatttttagtagagacagggtttcaccatgttggccaggatggtctcaatctcttgacctcgtgatcagcccgcctcagcctctcaaagtgctgggattacaagcctgagccaccgctcccggccactttgctaatgtttcttggcattgtctgctgccttttgaactggctttcccttagcctgggcccatgtttcttcgacctaggaagggccctttctttgtcttatgctgcgtcatgttagctgtttgtcatcagtgtcacaaacattttttccccggtacatttctgacctgccatacttggaatttatttgagtttgaggagcacgtcactatcgatgcaacgcattcactaagtcatacatcttttccccagtgactcacagagccttctttacaacgtgctgaattcccatttgtactcgatctttctgcatccatgttcctgttcagtcccgttgatcgttaggaaaggtgattttacatattcaggtacagttgtaaatgatgtcctaaagtgtgcctattggcatggaaagatattggcagcactctaaatttttcaagtggcatataaaatatataaaagcatatgcaaaaatcatgcatataaacctgcataggagactggagagttcccttgacccttctcaggactggcacagggggtggctcgttttctcggctgccactcaatcccttacgggagggaccacacgaacggacaggtgcgggaaccagagcaaaggaactctcctctctggcgggagcaggctctgcgctggcctcacggcagcctccaagcatattacaatgctcttttagctctgccatctgggagtgggtgtctgtgacccctggagcctcagaaagcctgtgttacaatcagtgttgagtgttaatcagctcagtggagggtcagggtgacagcctttacaccctgccctcttggtacctgagttcttgtccggcgtccagcaagaatcaggtcacacgaacgaattaaagggtggtgaatatggaggactttattgagctgtggaagtggctctcagcagaaagggaagctgacaagggggtacagcaggaagataattttcccctggagtctggccatccctcagccaaactcctctccaacatccagctgcttcctctcctctctttgctcagatgctttctcttctgtgtgtgtcccctttgtctggagtctggggttcttatgggcacaggatagggggcagagcaggccaaaaggcaacattcaggtgggaaaacagggatagttgtcactttgggccacgggtccaggcttgagtgtgaagccctcaccagtatttccctgcctcctgcctgtatcacatacattctctcttcattcctagtgtgtgttcgtaatccaggatgaagagaagggagaagtcttcattgaaccccttttttcatgcggtcatttacatagtagcaacagactgcaggatgatttcttagattccacaaattttttttctttttctttttgagagagtctcactctgtcgcccgagctagaatgcagtggtaccatcacagctcactgcagccttgacctcccggcctgaagcactcctcctacctcagcctccaaagtagctaggacttacatgcacttgccaccatgcccagctaaatttttttgtatttttggtagagatgggattttgctatattgcccagactggtcttgaactcctggcctcaagcagttctcccgccttggcctctcaagatgctaggatgacaggcatgagccactgcacccagcccacaaatgttttcaagttactgatctgccaagtttacaattccagtaagagtttgaaaaggaaataggaactgaaacctgcctgtgtttgctgaatctctgctgtgtgctaggtgctgaggtgctttgggatatgcattagaagcttgcttgttaaccagtgaccatgactgcatttgagctgttgctgttcacacatgggcatttccatcaggacagcacagccaggaggagagtggcggctccgggacctggggctcaggcgaggccttgaggagcttaccagaatagtgagggcccacgagggccaaagacccacaagtggtaaaggacaggtggccccactcaggaagacactttctcaggcagaaccggaatgacaatgggaggccagttgtggagagcctgggacgccagaataagtgagcacgagagaccgacaggatgagagccgcatttccgctgagacagtgtggctgcggggcacggggcgctggagcagagtggaggcaggggtgggaggatgcacctgggcaggacgtggtagggcagtggggctgggtgaagggatggagagcaacgccgcagtgttggttatccctttcatagttaatgtagtgtccttcacaaataagatttcttttattttcaaatacaatcagatacaaagtcagtctgcttttgagcggtttgttttgccacagtaggaaataatcgttgctggttcatgtgctaattttgttgccaaatacttcatcgtgacacagggggactaatcaatgttaatttccagtgttacagaagtggccggcggtaagctgttaatgctctcataaatgaccatttttcagaagttatttgctttgtcccggactctacctaaaccaatgtacgtctgcccccctacattcaaacatgacttccgttttgatcatttttgctggaatattaaaaatgcatctcaaaggcagctgtggtttctgggaagctgtgtttggcatcagtccttgttcacttttagcacttgaagctgaaaaaagcagtaatgtcaacataatgaaccatcttaattcagcctggcagaggtcacaacagctctagttttcaccttcatggtgaaggatgatcgtgttgttggaataaatagacctggacttgattacaagtgacatttgaaagtgttgattcagattgtcccgtcgcttcaaaatggagccctagtctttaagcacagtggtgagataagtattattaatgacaggcattagttaggataaaggcaaaaaaaaaagtttggaggctcaaatcattaagttggcagtagaaatatgaatagaaactcagctggagagttgactcctcgcactcctgtttgtcttgactgtgcctcagatggcgtctcgcgcccgtttggttttgtctttcacagacgtttgccagggaccatgtttttccatctcccctctgttttaacacagcgccttaccaatcacacaccaaattagtgcagtgattttgtgagcgtggagagagtaaatgaggagagttcttcaccagaaaaagacagcaaagacgtgtttctcttccttctcgtcacagaacaaactccttactcgagggtggagtatgtgtctcagctctccttctcttcagctctctctttgttttcctggggaaatcccgggccttgttgaaaggacctgcagcagctctgacttcccgaacactcacaggtgcccgtgttgaggttcccaatggcgtctttcagcccctgggccggcttgctttctgcgcagcgtgtgctcctgatgtagaggccgtggatactggcatttttttagtgcatcagctgatttctctggtgtccacccagggctcgcctcagaggatgtgctcagctcgcaaacctgtgttctttgctctttgcagaatggaagccctctccctttcggtgtgtatgggagaggccatagctaggatgttgagcctctgaagttgtaaagcttactacctttttatttattgtatgtttaatttaaaggatcatttagcattgcttgtgggcaaatcctgactaatgccagagtgggggtgttcttggatatagagcttgctttgtcattggacgtttgtgtgttagaattatgtaagcaataaaatattttagctgggcacggtggttcacgcccgtaatctcagcactttgggatgctgaggtgtgcagttcacttgaggccaggagtttgagaccagcctggccaaaatagcaaaaccctttctctactaaaaatacaaaaaaaaaaaaaaaaattagctgagcatgatggcacatgcctgtaatcccagctactcaggaggctgaggcacaagaatcacttgaggccggaaggcggaggttgcggtgagctgagatcacgccactgcactccaccccggcaacagagcaagactctgcctcaaaacaaacaaaaataataaaatatttaaaagtttgacctgaaaaatattgttacacttaacagaattttaaatgagaaagacctttttgataagaactgtcccacagtaaagtggatttttttgccaaaatgtccctggagataatttaggcagagacttaaagatgaacctcatagcggccatcagatcccaaggaggaattcatccctgccctcttgcccgccgcacacccacaaccagggaggggcattagagagcacagtgtaaacggaaacagcaaggaggctgaacagagggctgagaaatcaccgtgccatcataaagcagccagctcaagtggaaactcatcttaaattggggcctgccccaccagggctctgctgaattgcttttgatctcaaagccaaagcaagaagcataactgtagaagaatcgtttctacagtgttttcccgcagccagttggccttgccacagcggacctaaggagaggaaagaagggagggaagcccccttaccactttgcctttcacagatgccgtcctgcgcacactgccgcgggctgggctggagctctccccggggagcagctgggggcagcctgggagactgggtcccaccccagcacctaacctgaatttcttcgaggcacaaaggataaattgcagatttttcactgtgtctaaaggtgtgaaatgtttaacagctataatttaaaattcacttgaagtgaggagagagtgagctttctgggtaaagaggggcaggctgcaggcctatgctgttgaagggtgctgtctcctgatctggttccgatgcgctgtggtggaaatgtgtcagcatgcattgaagattcatatgctcttctgtatgtatgtaacactcagatggagaggttttaaaacatcaaaggggagcctagaccttctttaaaaattattgtcagagtagtgccgatactcatttaaaaacctaacatcggaggtttgaggaatctctcctctggtagttaaaactgttttttttgtttttccttaagaactatttttttttattatactttaagttttagggtacatgtgcacaacgtgcaggttagttacatatgtatacatgtgccatgttggtgtgctgcacccattaactcatcatttaacgttaggtatatctcctaatgctatccctccccgctcaccccaccccacaacaggccccggtgtgtgatgttccccttcctgtgaccatgtgttctcgttgttcagttcccacctatgagtgagaacatgcggtgtttggttttttgtccttggtgatagtttgctgagaatgatggtttccagcttcatccatgtccctacaaaggacatgaactcatcattttttatggctgcatagtattccatggtgtatatgtgccgcattttcttaatccagtctatcattgttggacgtttgcgttggttcgaaatctttgctattgtgaagagtgccacaataaacatacgtgtgcatgtgtctttatagcagcatgatttacaatcctttgggtatatacccagtaatgggatggctgggtcaaatggtatttctggttctagatccctgaggaatggccacactgacttccacaatgggtgaactaaaaaggaacgtattttttcccagcgtagcatctctaatactctaatactgtgctcctcttgttggctccggctgtccacagcctgggggctgggaagagagtgctgcctgtggaaatgctcgggaaccagagggttcactttctccttttgcatcctgggaggtgacaaggaggtcactctggatagccacaggaggagactttctaagagatggttgctgtgtttgttggtgtgaggggcccaaagttgaaattttatagatatacatcttcaatgttctgttttccctgttaacacccagattttccttttattcttaggaatccagcactacgccctgccctctactgactggaggcaggcctctgcctactttagaagttaaaccacccgataggccttcgagcaaaagcaaagatccaccgagagaagaagagaaagaaaagaaaaagaaaaagcacaaaaaaagatctcgaacaagatcacgttctcccaagtaccattcgtcatccaagtccaggtctagatcacactcaaaagcaaagcattctcttcccagtgcctatcggacagtgcggcggtcgaggtgggtgtgaagggggcagcacctctggtaccctcatgacccccatgtccttcacaggacacccagtagagctaggtagaacgtttaaaatcagtgccgctttcattaagcagacgcgtgtatgcatgtgcatgtgtgccctgcaagtccaagtaagatctttttcagatttttgtttgttttatacttaactttttcttttttgagacagagttttgttcttgttgcccaggctagagtgcagtggtgcgatcttggctcactgcaacctccgcttcccaggttcaagtgattctcctgcctcagcctcctgagtagctgggattacaggtgcccaccaccacgcctggctaatttttgtatttttagtagagacggggtttcaccgtgtcggccaggctggtcttaaactcctgacctcaggtaatccacccaccttggcctcccagagtgctgggattacaggcctaagccaccgcgcaggcctatacttaacttttcaaagttcataaactactgccaggtttttaaaaattggtttgtttaaattctaatggttcctggaagcaagcctaccacatttgccgattgtgtgaaagattcacagggtggtgtgctgggggtcttttgttttatttgtataagtgaagtttcccatgctaatttgtctcaaatgtgtaaagttgcaagacaggagaactctttagcactggttctgggtttggattctctgctctgcacacgcactcaccggcaccgcactctgcacatacactcaccggtgccacactctgcacacacttcgtgtggcaccggtgagcgtgtgtgcagagatgcagcgacggtgagtgtgtgtgaagagggcagcgcggatgagtgtgtgtgaagagggcggcgcgggtgagcgtgtgtgaagagggcggcgcgggtgagtgtgtgtgaagagggcggcgcgggtgggtgtgtgtgaagagggcggcgctggtgcagaatgtttcctctccaccctccctccaggagtcactattaaaccaaaggccttcttgatgaggagccagtttttcagaaagcaggttaacatttctggcagcagaaattaaaaatgtaaaaacatttaagagtcacagaatttacatcttggtgaaaaccactttttaaaaacaaaacagtggctgacctacaggaggttggcacagcttgccctgttttcagaaccccgttacaccttgggttcgctgctgaacactggctgactctcctcggtttctctaacgccgcactgactgtgctcatctagtttttcttctggaattggtgttagctcttatgtttctgtgggaaaaatacacatgccttgggagctttacgggctttttaagtgtaattttacacatttgcctctctgaatatatcctaaaaacaatatgcttgctttctttacttatttatttatttattcatttatttatttagagacggagtttttgctcttgtttcccaggtgggagtgcaatggcacgatcttggctcactgcaacctctgcctcccaggttcaagtgattcttctgcctcagcctcccaagtagctgggattacaggcatgtgccaccacgcccagctaattttgtttttttagtagagattgggtttcaccatgttggccaggctggtctcgaactcctgacctcaggtgacccacccacctcagcctcccacagtgctggggttacaggcgtgagccactgtgcccagcctgctttccttatttttaccctggccaacacttaaagtttgacaagcatttacactcctctgcagtgaaattggatttgactccatgataaatcaatttgatctttcactctacatttttgcgagtgttttaaacgtttcatcacttcatacccttatacacgcaaaaaagaaaccttgctattttctaatcaaatgaacagttttgctaatatatcttcaatttttgaaggctcccaggaacttgtattgtatatcgaagctttttaaaaatttctcatttgaggccaggcacaatggctcacacctgaaattccagtgctttgggaagccaagatgagaggatcactttgaggcctggagttcaagactagctttggcaacatagtgaaaacctatctctacaaaatatttttttttaattagccaggcatggcagtggatgctttgaactcctgagctcaagcgtagagtctgaggtggaaggattgcttgagctgagctcaggagtttgaggctgcagtgagctatgatcacgccactgcactccagcctgggtgacagagcgagaccttgcctctaaatgcaattaaatgattaaaataaaaaatttcccacttgaatatgtttcttacgacattacatagctgaagataggcataaacaagccctcctagtaaccacattcagtaaaattcttcccaattttccttttctacaggctcaaaaggaagcataattccttcctaaatcccaaaccttgggggaccgatcattgtaagagctgttcatggtgtttctttagcgtaagaaattagctcagctttcatgtggggagtttttgcaaacacagcggatgtgatgtctgatatttccgggtatcctaccattcacctctaaagacaggtgatgccgtggcccccagcttttcccacattggcatattcagagctgaaaggcttcacctaacacttggaatttcaggtttctaagttgtacatcctttttgttgactggtctatagtagaaaaggtcattttacatattatttgaatgatttattttagaatcgatttagagttacatatttttgaataatttagaatagctttagttacatattacttcacatatgcaaatatatcttattatttttttttttttttttttttttgagacagagtctcgctgtcgcctaggctggagtgcagtggcgcgatctctgctcactgcaagctctgcctcccgggttcacaccattctcctgtctcagcctcccgagtagctgggactacaggcgcccgccacctcgcccggctaattttttgtatttttagtagagacggggtttcaccgtgttagccaggatggtctcgatctcctgacctcatgatccacccgcctcggcctcccaaagtgctgggattacaggcgtgagccaccgcgcccggcctatattatttttatatattcactgctgacaagtccaagaagcaaaatcctactcatttgtttgtaactttcagttaaaagaaaaaattaaggtaaaagttacctgagtgtggtttccaccgtgatggtaggctaccaattttaatccgacctacgtttaaaacactttacagcgtcagcagagcaaagtgtttccagaacactccaatttttaattagtctccatggccaaggaggtagtatctacatacttctagttaattttagttaaataagggatttaaaagcatttgattttgcaactgagacaaaatatgaaggcaaagtgcaagcttattataaaatgaaaataatattataaaacaaaaccttccaggtgttggattgtctagcaagttctaccgtgggtgctggcccctggcattggttcccctccacagggccaagggcatagctgggtgcagagaccggcagtgccgtggtctctggagtctgaggacataagttaaacaagctagtcaagccccagatgcttgggaggcagaggcaggaggattccttgagcccaggagatcgaatctagcctgatcaacatattctctatgacaaaagaacaagaagaagaagaagctggtggtttctcaccataaccttttcttgtggaattctgcctcagctcttctgggaacagtgagtgcgtgttttatttagtaggattgcatttttctaaactggctgcaaacctgcctcctccatccaagctctgccagcaataatcatttccagggatccaagtggctttaaaatgcaagttagaaatgggaggggtggtgatctcctcagtaatatgaattattggagtataaaagataactaaattttaaccaaaatattgaaagtgttaatgctgttgttatcagatagaataaactgttacaaacgcagcctccactcagaatggatcggacttgtcacttgggcctgaacagacctaattgatcatttttcatgactgctgccagcccacagtagaataccgcagttgttaatatttctaattgggtaggatgctacatggaatgtattttgttttatatattaaattactaaaattctatataaaatacagaaagttaagattagaaagccttcttacagcacaacgaatatttatttaatggctatactgttcctgtggttgaagtcccatgtatttagtatgtctaagttatgggcgactctggatctccaaaggcaaattagtcatggaagaatctttagttttggaaaatcactatgttgcttctcaaaaagtatactagttacgacaaggtagtatttagtgtcttttacatcaacattgaggctggcacggtggctcacgcctataatcccggctcttaaggaggctgatgcaggtggatcacctgaggtcaggagtttaaggccagcctggccaacatggtaaaaccccatctctactaaaaatacaaaaatcagccaggcgtgttggtgtgcgcctataatcccagctactcgggaggctgaggcaggagaattgcttgaacccgggaggtggagattgcagtgagccaagatcgtgtcactgctctccagcctgggcaatagagcaagactccgtctaaaaaaaaaaaaaaaaaaagattaaagtaaaatacttttattgtctgttttcatttgtattttgatattgtatctggttctctatgttaatggaatgaagaagtactcatgtagttcatttacaacctgaaattaaattttaataagtatcagcttgaaactaagtttatttttaaaacttttgctaagatagtctcttgtgttcatttagttatctaaatgcatcttcagagttagcctgggcttctgggagttctagatagatctttgaatgttgtcattttaagatatcttccagtatagagagctatatgataaaaatatatttctggccgggcgtggtggcccacgcctataatcccagcactttgggaggctgaggcagacggatcatgaggttgagaccatcctggctaacacggtgaaaccccgtctctactaaaaatacaaaaaattagccgggcgtggtggtgcctatagtcccagctgctcaggaagctgaggcaggagaatggcgtgaacccgggaggcggtgcttgcagtgagccaagatcgcgccactgcactccatcctgggcaacagagtgagactccgtctcaaaaaaaaacattttatatatatatatatatatatatatatataattctttgtagaaattagctccctaaatacttggggttggtgaaggagactggggatttggaagacttttcttaggagtcttgtttagcattcagaagggactcaggccacactgggtttctattttaggttgaaagttgtggctcctcactgccctttttacccacaataaattgcatagcaaatccgtaaaagcgatgactcatctcctaatcctgccccttaaagggggaaaccagatgcttgcagttccccaagtggtagtgttgatcatgccaaggtgaggaccgtcgttccatcccttgcaaagtgaatcaaagtgaattgtagccaaacacagataagaccagagggtgtctgcactgagcagtccaggaaggaggggagctgcagtggctgtcaccgggctgggacacgaggaggaattgcaggtgaaatcagatccagtttcaacttgaggaaaattcagccccgggagctgctggtagagcccagaccttgatgctgagtcatctgcacagagaattccgtgacagaaaggccgtgggtagagacgtgaatggaggaagtggagtagatgaaatggttaaatgttggagaaaagaggctatttatgaatatgaccactgtcattcagataaaatttctggactgttatcattgaaaaaagtctcattatgtttctatttgaaagcaaaccattatgcttttttgaggaaaaaaaaaaactgtgagtcacgttatgcttgcaagtgtttaattcagaccatttcatctttaagaaggcccctggtcacattatacggatgatttgcttattaaatggaactcctgtttcttgcaccatgttgtggggtcaatatgagaagcctaattaacagaataaaaagcattaaagcttctttagctaaagtcaaacttagagaattgtctaatggtatgtagcccctcgttctaagatgggcgttttccccagataacttgaaaatctactggtaacagccacttccctttaaagaattctattactaatagccatgacaaaatggtattgtatttcaaagttaagaatttgcaggccttaaaaactaacttatttttcctgattattgagtttattgtagaattctacgtgtaagcattccccagccgctatagctttgaataagcagagcttttttcagagttctggtagcgcccagcccagcaccttttattctgaatgtgaagtgtgtgcctccgtgtcacagagtcacagcctccccagggacgctgcgcgcggagccctgtcagagcagcgcgtcagtgacagcggcagccgagccaggaagttatcaggcagcctcgaccaccaccagatttgactccgcgagctcttttgagggaaaacctggtaaaacgtcaaggtgtctaactgacctcgcctttatcatctgttctgtaaatcttaggaaaggtctgggaaaaaatcaaaacgattctgtccgttaaagggcagccactcctggccctccaggatgccggggtctgagtgatcccgagctgatctgcagaagcacagcctgtggcatttgcggtttattgtcatgaaaatgattcaacgtagaactttttcaaatggcaaaatcaaaccgctcttctttatattgtttttgaatgagttgtcatggaaacaaaatggaaataaatggtgttttttttccagatttgtgctcattgcaggtcttcccaaaatagtagctttactgaatgaacaaagaactaaaatgaaggtcccaaactcatcgctaaggggcctccactaaagagcatcacccctggaggggcgcgggtctcagggtccttggccgcgtgtggattatgtcaccacaggagagggacgagtcctttccaggcacatgaggaggaggaatcagtgttaatgggtggctttgcatctgtgaaatcgcataaacttaagttagctgaagctgtcgtgagactggcatttccaaattggattgaaggtttcaggcttcatgccagcgcaccacagcctgttcctgagtatctgtgctgagaggctgtaagattagtgtgaacaggagaaatttccaggtaggcctctagcttcattaccgttgggtttcttactgccggtattcagacaggtagacatgactcgctggagtttgattgccttttcttacctcatgttggtagaaacatcaatgagctgaaatgtatagggagataaaatgggcagaggcaggaggaaggaagaggaagcgccagcctgaggtggtcatgaactgcatactcagaccgtggctcatggggaattggttgccattgaccacgtgaagcagctccagcctccacgccagttgcatgttggttaaaagtttgtccttggtgcgataagtgtgtggaacgggagagagaccatctctgcctctgagattggattcgggtttcagttcgttgtcggtaaagtagtgaagtgtggcaggggttctctgaagcctcagggtctacacaggcaccaccctgaggagcagcctctgcagacggggcctgatctctgccagggcagtaggaagcatgacacgtcccgccagccaggccacagagctgaacactgcctcctcccctgtccaggtcccgctcccggtcccctcggaggagagcccactcccctgagagacggagggaagagaggagtgtgcccactgcctaccgcgtgagccgcagccctggggccagtaggaagcggacccgctccaggtaggccactgggtgtgcacgcaggtgctggatgtgggccaggtttccctgggtggaaagggcgtctgaaggtcgggtatctgtgagcagagctgtggatgaccagagggaggtgctgagtcccccaccacccccccacccccagtggcatggccatcactgttgacacttgatcacactgagctcctgtgtctggtgggcgggggtcacttacccaccggggctctgcacggcctggcttcgtgtccagctttccactgtgctggtacctcggctgggtccacatgcagctgctgcccctctacctgctggtggagaggacaggaaggcacaaacagaaggaaaatgcaagcttccggtcctaaagcctcctggtctcaagggcagtcactgtggttgcctggctgctgtgtgacggtgactacggcccaggctggagctcccaggagaggccacagagtcctgttggggcctagagggcagggagcatccatcgcttacctcttgaccactaaggagagcctgtcttggttggagcaggagatggagggaggttagcattcatgttcatcaagtagaagccccagccgtggtgcctggcagggcctctgacagcccagggtgccacgggctcacccctcactcagtgcctggcactcagtagaggttccacctttcacttcaggaaataggtccaccatctgtccgctcaccccggcttccagtagctgtggacggccacctccattggtgccgccagtgagcactaccctctcggccgtgggggtgccatctcacgagcgcctcctctggttctcacccactgatgtcaccacccagtgccttgcgtggggcagccgtgcatttccactctttccaagcacaaggagcttgttttgtgtccccatgtggagttcgtgcagcctcctggctgtgtgggtggaccgtgtctgcgtctggagctacacagagaaggatggagcattgcacatcgtagccttgagcttcataacacggcactgctaagtgcatgggggtcaggacactcagggtcccagagccttccagaggacgagccttacattgccaggatcacccacacactgggaccctcctgctcctgggacggatggtcccagccatcacccacactgcctagccacaaggcacacactaggcagagagccacagcaggtcctccccacagcaccctgggcaagaagaccgtgctgcggttggcctagtaccacggttccctccgttgacaagatgtgatttttttcttaaaacagaaaaattagcaaaggaactataaagcggatagataccagcaatgtttcatgtacacctggctctgtttataaattacattttgttccttagtaatcctacactgagcattcatgtctgctctcatacaatctgatgaaaattaaaatgttagcatccatcccttaaacaagtaatttcacatcagaaattcaccatcacctttggtatatgtgaagggcatggttagaaattaattccgtctcaacagaagaggccttgctttgccttcacattaacctttgctttaagagagacctcgtgtgagcaagtagtgattgtatctggaagtagcagcgtcctgatggccagccagcacactcagacgccagactcgcgtgacctgctgacattctcaccgagcactaacaggtcacacaagagaagcaaagggttagactcagtgcagtgctgagccctgagctgccgtgcccagacagacggaattaaacctgcaaaccaaagtctgcggagtgttaaactgtgattcactaggaactcaatagaggtgaatacgtgtgtaattactggttaattttgtattcttaattacaagcccccagttagtctataaatccagaatatgggtttggttttgttttcttttgggggcgttttttttttttgagacagggtctcaccctgtttcccaggctggagtgcagtggcgtaatcacagctcactgcagcttctacctcctgggctcaagccatcctcccacctcagcctcctgagttgctggggccacaggctgtcaccaccatgcctggctggctgctctcaagctcctggcctcgagtgatcaacctgcctcaacctccaaaagtactgggattgcagacatgagctcccatgcctggtacagaatatgttttattagcaatcattatattaatcctacagccagcccgtgtccctgtctcagagcgggcgtccacttccttgctgtggcttagtgcacataattcagctaccaagttgctgtcactttaatgctgtgacagcaccagaccaaacccagggaaatgcccactaccgagatttgctgctttttttctttttcttttttatttttatttgagatagggtctcactcccattgcgctggctggagtgcagtggcacaatctcagctcactgcggcctcaacctcctgggttcaactcgtcctcccacctcagcctcctgtgtagctgagactacaggcacatggaaccatgcccagctaattttttgtatttttagtagagacagggttttgccatgttgcccaggatggtctcaaaatcctgagctcaagcagtctgcctatctcagcctcccaaagtgctggcataaaccaccatgcccggccctgaagggtcatttctgtaaactgattattgcctgattctttcactgacttctcacttggaaacttttttaacttataggcaagtttttaaaatagtacaatggggccagattcagtagctcacacctataatcccagcacttggaggccaagatagcaggatcacttgagctcaggagttggaggctgtagtgtgggctgtgatcgtgcctgtgaatagccactgcaccccccacctgggtaacagagtgaaaccctctctttcaaaaaaaagtgtacaataaacacccatatgcataaaatctgtagctcagttccacaagagctgacattttgccacattgctctctctcaccccttcccatcccgcccatcccatccactcccctccctccctcctccgttcgtgtgtgtatttcatgaccttggcattcctgagaattccaggccagctccactatagatggtcccacagttgggcttcgtcttgctgtgtccccgtggctgggttcagggcaaatgttttggctgcgtaggcgacattgcgtagcttcccattgcatcacagatcaggacacacagaagtgtccatttgtcccatcattcatgatgctaagtttgaccacttgattaagtctgcatctgccccttcgtctccccaccagcgaggaatccaggaggtgacactgaagcagcgcggctctcctgctcccagcagctgtcttctcatttgtctcagcatccctgggtgacccctgcctgaatcagttcttacactgctgactgcaaaatagtgactttccccctctcttcttccttctgtgtttatgctgaagaccctgcccctttgtttaaatctcaccgtggactcaggagcatttttggttttgattttttatttgttgtgtgataatccattgctattattattctattagatggtgacattgtctccagtttggccagtggcaacccttccaagtcagttctgttcttttgacacctcccatagttctttgcattcttgcgtttggtacaagatgttccaggtttactgggcattttccctgctccagccctggaatctaccatttcttcaaggacctctggttccttttagtgaatatttgaaaatccagatgtggacgtatgaggaatttttaggagtaaaatttggtacagtgtggaaatatataaaacaacattcatgaaagttattttgagtatgtcataaaagtgtttttcagccaggcacaatggcgggcacctacagccgcagctacttggagggctgagtggatctcttgagcctaggagttcacatccagggcttttcacaagaatattgaccaaatcttctggtagcacacttcaacaagatgtcccggttatcttattgtagcaaatacaatgaatgattagttacaagtttttcccattgagtttctagtacttaacactgcacgaggcacatggacaactgtttgttgagtgagtgaatgggagttcactgctgcagtaaagatctgcctttatacatgaaatgttaattccaggtagactttgctaagcgaaggatgcataacctaattccctagagcaaccactaaaaacaaaaatgtagctaaaaagccaatagcagatataaagtaggattctagatgctttcttaaattcatgaaacagcagaaaagggcaggtgggggaaagaacaaatgggacaaataaaaacaagattgtagacttaaaaccatctgtaaaataattacattaaatgtaagaagactaaagactagttaaaaggcagtgattgtggagtggattaaagagcaagacctggcctggcgcggtggctcatacctgtaatctcagcacttcaggaggccaaggcaggtggatcacctggggtcaggagttcaagaccagcctggccaacatggtgaaaccccgtcactactaaaaatataaaaattaggtgtggtggcaagtgcctgtaatcccagctactcgggaggctgaggcaggagaattgcttgaacctgggaggcggaggctgcagtgagccaagatcgtgccactgcactccagcctgggtgacaaagtgagactctatctcaaagaaaaataaacgaaacttttccaccaaactccagtcccagatggcttcaccagtgaattctaacattcaagaaaggaggggccaggcacgatggttcacatctgtaatcccagcacttcaggaggctgaggcaggtggatcacgaggtcaggagtttgagaccagtctggccaacatagtgaaactctgtctctactataagtacaaaaaattaaccgggtgtggtagtgtgcgtctgtaatcccagctacctgggaggctgaggcaggagaataacttgaactcgggaggcggaggttgcagtgagccaagattgcgttccagcccgcgacagtgcaagactccgtctcaaaaaacaaaaagaaagaaagaagggatactcttttttaaaaaatagatgaaggaacacttcccatctcatctcttgagtccatcataactctcatacctaagccagataaggattctgtgtttgggggagggggtgtgcacatgcacccttgtctgttcacagatcagtactgtgtgcacccgtgtgtgttcacggatcagtactgtgtgcacacgtgtgtgttcactggtcattactgtgtgtgcacccgtgtgtgtgcacagaccagtacagtgtgtgcactcgtgtgtgttcacggatcagtactgtgtgtgtgcacgtgtgtgttcacggatcattactgtgtgtgcgcccatgtgtgttcacggatcagtactgtgtgtgtgcacgtgtgtgttcacggatcgttactgtgtgtgcacccgtgtgtgttcacagatcattactgtgtgtgcgcccgtgtgtgttcacggatcattactgtgtgtgcgcccgtgtgtgttcacagaccagtactgtgtgtgcatatgtgtgtattcacagatcagtactgtgtgtgcacccgtgtgtgttcacagaccagtactgtgtgtgcatatgtgtgtgttcacagatcagtactgtgtgtgcgcccgtgtgtgttcacagatcagtactggtgtgcatgtgtgtgctcacagaccagtactgtgtgtgcatatgtgtgtgttcacagatcagtactgtgtgtgcacccgtgtgtgttcacagaccagtactgtgtgtgcatacgtgtgtgttcacagatcagtactgtgtgtgcgcccgtgtgtgttcacagatcagtactggtgtgcatgcgtgttaacagaccagtgctgtgtgtgcacatgtgtgttcacagatcagtactggtgcacatgcatgtgtgttcacagaccagtgctgtgtgtgcccataagtatatgttcacagaccaggactctcaagaacatagatgcaaaaatacttcacaaaatattagccaactaagtattactgagactcctgttctccacaagttgacgcagagatgcagtgcagtcccactcagagctcccacggcttttctagaaattggcacacaaactccaaagcgtgtgtggaaatgcagatgacctgggagacccaaaacaacctccttgacaaagagcaggatttcaagacttaccagaaagctacagtaaccaaggcagtgtggtgtcagcatgaggatacaatagagcagtgggatggaatagaaagtacagaaaaaaaattccatacccaaagggcagggggccgggaccacagccacagcgattcagtgaggaaaaagagaaaggaaagtcttttttttttttgagacagggtctcactctgttgcccaagctggagtgcagcagtggtgtgatctcgactcagcccggctgactgcagcctcctgggctcaaggaatcctcccacctcagctgggaccacaggcacacaccaccatgcccagctaatttttttttattgtgtgtagagacagggtctcgctatgttgcccaggctgatgttgaactcccaggctcaagcagtcctcctaccttggcctccgaaaatgctgtgactgcaggcatgagccacagcacccagccaggaaactctttccaacaaaacttgcatgaacagctggatatcggaatggggaaaaagtgcactgcatgctgtatgcaaaatttaattcagggcggatcagagatctaaacaaaaactagaaccattaagctttttgaagaaaacacagaatatgttcatgaatttgagggtggcaaagattccttaagatgtagaaactcctctgataagaggaaaaaaccaattagacttcattgaagtttaaaaacttctctcaaaaggcacagttaagaagatgaataggcaggccgcaggctttgctgcatgtgtctctgacaaaagcctgtgtcagtaccaaaaagacaaaggacccaattagaagggggcagatgaagccagccgacttgacagaaggatctcttaaatagccggtacacacatggaaagatgtggaacggcatgagtcaccagtcagggacgtgctgatgcaaccaacgagacaggactagacgggggtcacccgtccctaaaaaccaggacgggctcgggggagagtgggcacgggcccagcggctgcgctctcagacactggattgggaaacgtgtgcagtttcttgtgacgttaagtacacacctactccctgaccagctgtcctgttcctagctgtgaactcctctataaagtcaacatttaaccaaaaacactttgattcataattaccgaaaactggaaacaaccaaatctctattaacaggagaatgaatcaacagataatggtagcgtcctgtcctgtaatactattcatcggtaaaaggaacaaattgaggatcaccctgcgtcgtggaggagtctcagacatgctttgctgagcaaaagcagccagacacaggccagccacagtggctcacacctgtgatcccagcactttgggaggccaaggcaggaggattgcttgagcccaggatttgcaggctttttttttttttggtagagacccccatctctacttaaaaaaaaaaaaaattagccatttgtggtggcgtctgcctgtcgtcccagctacttgggaggctgaggcaagaggatcactggagcctgggaggtcaaggctacagtgagcagggattatgcccctgcactccagtttgggcaacagagggaaactgagaaacaaacaacagaaaaccaagaagccaaaccaacaaacaaacacagacatagcgtggggtttgtctacatagagctttaagctgtgtcctagaaaccagagcagtggggaacgctgagggtggagaaggggtatagacggacttgaagtggcattgaggagccttctggaatgaagggacgcccctgcgtggataaggcccaggtgtcagggtgtgcgcacttgccaagctcagcggcagcaccgaggacagcgtttcacccaatggacagtggcacctcggtgctttaaaaaaaaatgaatgagttgctccattccttcagcaagggcttagatcagattgtagcagaattgaaccagtttgcagttaaggattagtaacctgccttttgttcattatgcagccacataaactcagctggatttggggagtaagtcattttggacacatgtcacatgctggtatatgttttatttatttgccgcttcctttgaaatcctggcatgtgtttacagacaacaatttcacaaaacattttgcagtttagaaaaatgactctttcgtgcaggtcccacatgcgtgtgttgaacagtaaacaacatgttgtcctcactgggcacgtcaggcaggcttccagaagatgccaagtcatctgcccgggcccagctcaccagggacagcccctccagcagctggatttaagctgccagcgagcaccgtctctggcaggtcccgccttgtttgaatggagctgggtgggagcgccacaggtctggcgctgctgcttaggtcacttcactggcaccaacacagtctgctcacgcccagaaccacacaagggagcccggacagaaacgctcagtccccccctgcatatcggggctgtccctaccagggcatgctgtggtccctggctaccgcagctctgtctaagttctgcagggccagacactggtgaggtcctagagatgggtagagggcacagcccctcgatggggtctgcaccccagactctgagcacagccccagccattaagcaagaatgtcccagatatcggggggtggcacaagaaatgcatgaagtccggaggccctgatgaggggcagggcttggggtaactgggcctgtgcacaggccctggaggtctccctggaaggcagaggaggccaggctgggaaggggcttcgtggcacgcagaatcataagggaggccagacgcttgcagctgtgcaaatagcaaccccaggagagagtcagacaccagcagagaaccacggttcccccttcaggttggcacattgagcagtttgggtccacctggataacgagcgtgaggctgagccagggagtccccctggcagcttctgcagcagagggccccgcagccctactcctgggatctgtcctgcccaggcaccagcaagcaggacgggaggggagggataggggaggggaggggagagggggaggggaggggaggggagagggggaggggaggggaggggagcggagagggggaggggaggggagggaaggaaggaaggaaggaaatcagtgatgcaaatgacccatgcaaagactctccaagaaacactgtactcagggccagaagcgcaggctgcagcgtctgttacagacgaattctgaaagaagatgccaggtagggcacctcagggcctggagggcctcacaggaagggctcaggcctgtctgcctttaccaagtacatgttcactctcttaggtgtttgtaggggagtggccaagacagccacgtggctcaggtgtggaatgaagctagaccaggtggaagccgaagggtcggcctctccaggcaggagagaaggatgatctaagggcaggtgcaggccagaatgtctggaaagcatttctggtgcgggattgccagtttggtgacgtggactctgggaagcaaggggacaggggacagcagtcagagctgagctgctgcccacagagcaggctccactgcccagaggctaagcggtatcaccaagcggcggacaactggcaggtcaggaagaagtgccactccagcctggacaacagagtgagaccccatctcttaagaaaaaggaagaagcagcaccagaagctgcgccccctagtcttaactgtctgggaggctgaggcaggaggttgcttgaggtcaggaggtgaaggctgcagtgagctgtaatggcaccactgcactccagcctggacagcagcacgagaccttgtctgttttttcaaaaaaaaggaacactaaactttgatgtattgatactttaataaatttcctgtatctttttggaaatttttattgatgaaacataagtggcaaagcactatgaactgcctgtggtggcttatcttaggtattttacatgtaaataaaatgctggttgcatcttaaataccacaaatattttacttgaggtcctaaatggggacgcgtcatctgttatcagttaaatgaaataagtagctttaagagaagttaatgggtttggagtggttccgtccctgaattgtgccttgatgaactcttagccaaaaactggctcagatccgagcttctccctttgtgccctgcctttaaaccaaagctgcatctctcacagaaactcttgcctttcagaagtccccacgagaagaagaagaagaggcggtcccggtcgcggaccaagtccaaggccaggtctcagtcggtgtcacccagcaagcaggcagcgccccggcccgcggcccccgcggcccactcggcgcactcagccagcgtctcccctgtggagagtcggggctccagccaggagcgctccaggtaacccctgtcctccagcagctctctctggggaaaggcaaggggcggccagcaggactctccctcctccctgagtccttgcctatgtcagtactcgcctgtgtccagggggcgccagccacaaagccaaaccgcaccccctctagcaaggaagtcgccctagatgtggcttctcacaatccatgagcgctcagaggagcaggtcctgtactggggagaccctcctgcagagcccaggagtggagcagtccacttgaagcagcccaagtgtcacacacgtgcctgatgcccaccaggcacactgggctgtgcaatgaccagtagaccgggaactgtcaccaggtccccaggctgccgtggctggagcaggtccccaggctgcaacggccagggccaaatgacgccaacctgtcaccgggcatcacacctgggcagcagcacagacgtgggcgtcccagtcccgggctaggtgataatgacttcaagtcagacaccctccgctgcccaggcacccacaccctggggggaccagagagggcagcatctgggaacagctgctccctttaaactgattgcttccataaatgtcaatcatgggagtaacgcgcaactgttccattctagtggcagaggcctcagctaatttgagatggattagaatctaagaggtggcacctttagagttaaaatgtaaatcaggctgggcgccgtggctcatacctgtaatcccagcactttgggaggccagggcaggaatttgagaccagtctggacaacatggcaggaccttgtctctactaaaaataggtggcacgcgtctgtaatcccagctactcaggaggctaaggtgagaggattgcttgagcccaggaggtggaggctgctgtgagccatgacggcaccactgcacatcagcctgggtgacagagagagaccctgtttctgaaaatgtaataatgataaaatgtacatcagtgtaggaggctgagcatcgctgcggggagggggtgttggctccagcacacagacgcctcatgcacaggccgagggcacctacagccaaggccgtggttctgggaaggctccaccgttctgctgagtctttcctttctttgtttcttttttcctttgtgtttaaggtaattttatatgaaaatctttttgagttagattgcaatttgtaaacatttcagatgagtataacacagcatgtttatgatgccaagttttattgaaggatactggaggggtgggcgcggcggctcacgcctataatcccagcactttgggaggccaaggcgggtggatcacctgaggtcaggagttcgagaccaccctgaccaatatggtgaaaccccgtccctactgaaaatacaaaaattagccgggcatggtggcacacgcctgcaatcccagctactcaggaggctgaggcaggagaattgcttgaatctgggaggcagaagttgcagtgagctgagaacgtgccattgcactccagcctgggtgacagagtgaaactcttgtctggaaaaaaaaaaaaagatactggaagcagatgcagtgggcacttctcagttctagagttggggttcggaggtggggatgctgttcactggccttggctcagcatcttcacacggttgtaagctctgctctctctctctctgcattaggggagtctctcaggaaaaagaagcccagatctcttcagcaatcgtttcttccgtgcagagcaaaatcactcaggtcagtgggcacgcccccctcccgctcccagcctttcatcaaggggcctcgtggtttctctgttgctaattttcattccctgtccctcctgtccctgtcatgggacagggatctcgggcaaaataccacaggctctgggtgaggccgagggcaaagccgtgtggcccgcaccctgcacagccaggctcctccgccgcccccacggtgctagcaccgtctggtcttgaccaccaactcgttgatgaatttcttcaccacgtgggttgtctggccaggtcttcacaggttctcctctgtgtctcgccctgcacaggatctcatggccaaagtcagagcgatgcttgcagcttccaaaaacctgcaaaccagcgcttcctgagacggggccagcggaggcagagccgggaggctgcgtgggcttctgggcaggctcacgcagacgccggccacaccatccacctggccgcctccatggacccttggtggcttttgtaaattaatttttgatgacattttgagttttaagatttctgaccagcagtctcttacctgtatatttgtaaatatatcatgtttctgtgaaaatgtattatgaaataaaatgggaggaaacaccttttctagctag

SEQ ID NO: 10 - DNA, 1008 bp, Homo sapiens
atggatcccaaggggctcctctccttgaccttcgtgctgtttctctccctggcttttggggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagaggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtgtgacacttccagagagctga

SEQ ID NO: 11 - DNA, 1405 bp, Homo sapiens
aggagccttaggaggtacggggagctcgcaaatactccttttggtttattcttaccaccttgcttctgtgttccttgggaatgctgctgtgcttatgcatctggtctctttttggagctacagtggacaggcatttgtgacagcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaata

SEQ ID NO: 12 - DNA, 618 bp, Homo sapiens
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactcctcacaagacagaactggtatga

SEQ ID NO: 13 - DNA, 1464 bp, Homo sapiens
atgagcctccccaattcctcctgcctcttagaagacaagatgtgtgagggcaacaagaccactatggccagcccccagctgatgcccctggtggtggtcctgagcactatctgcttggtcacagtagggctcaacctgctggtgctgtatgccgtacggagtgagcggaagctccacactgtggggaacctgtacatcgtcagcctctcggtggcggacttgatcgtgggtgccgtcgtcatgcctatgaacatcctctacctgctcatgtccaagtggtcactgggccgtcctctctgcctcttttggctttccatggactatgtggccagcacagcgtccattttcagtgtcttcatcctgtgcattgatcgctaccgctctgtccagcagcccctcaggtaccttaagtatcgtaccaagacccgagcctcggccaccattctgggggcctggtttctctcttttctgtgggttattcccattctaggctggaatcacttcatgcagcagacctcggtgcgccgagaggacaagtgtgagacagacttctatgatgtcacctggttcaaggtcatgactgccatcatcaacttctacctgcccaccttgctcatgctctggttctatgccaagatctacaaggccgtacgacaacactgccagcaccgggagctcatcaataggtccctcccttccttctcagaaattaagctgaggccagagaaccccaagggggatgccaagaaaccagggaaggagtctccctgggaggttctgaaaaggaagccaaaagatgctggtggtggatctgtcttgaagtcaccatcccaaacccccaaggagatgaaatccccagttgtcttcagccaagaggatgatagagaagtagacaaactctactgctttccacttgatattgtgcacatgcaggctgcggcagaggggagtagcagggactatgtagccgtcaaccggagccatggccagctcaagacagatgagcagggcctgaacacacatggggccagcgagatatcagaggatcagatgttaggtgatagccaatccttctctcgaacggactcagataccaccacagagacagcaccaggcaaaggcaaattgaggagtgggtctaacacaggcctggattacatcaagtttacttggaagaggctccgctcgcattcaagacagtatgtatctgggttgcacatgaaccgcgaaaggaaggccgccaaacagttgggttttatcatggcagccttcatcctctgctggatcccttatttcatcttcttcatggtcattgccttctgcaagaactgttgcaatgaacatttgcacatgttcaccatctggctgggctacatcaactccacactgaaccccctcatctaccccttgtgcaatgagaacttcaagaagacattcaagagaattctgcatattcgctcctaa

SEQ ID NO: 14 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 15 - DNA, 3150 bp, Homo sapiens
atggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctag

SEQ ID NO: 16 - DNA, 3126 bp, Homo sapiens
atggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggatccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaa

SEQ ID NO: 17 - DNA, 2436 bp, Homo sapiens
atgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaactaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataa

SEQ ID NO: 18 - DNA, 3245 bp, Homo sapiens
attttgtggcccgctatggcggcggtgttgaggttgggtacgggatgcggggtctttgactgaaggggtaggccaagtggaggtatcagggacgtcgcgcggcacagaagaggaccagcctggacgccggggacgctgtcatgtacggcgcgagcgggggccgcgccaaacccgagaggaaaagcggcgcgaaggaggaggccgggccaggcggtgccggcggtgggggcagccgagtggagctcttggttttcggctatgcctgcaagctgttccgggacgacgagcgggccctggctcaggaacagggacagcacctcatcccctggatgggggaccacaagatcctcatcgacagatatgatggacgtggtcacctgcatgacctttctgagtacgatgctgagtattccacgtggaacagagattatcagctgtctgaagaggaggcgcgaatagaggccctgtgtgatgaagagaggtatttagccttgcatacggacttgcttgaggaggaggcaaggcaagaggaagaatacaagcgattgagtgaagcactagcagaggatgggagctacaatgccgtggggttcacttacggtagcgactattacgacccgtcagagccgacggaggaggaggagccttccaaacagagagaaaaaaatgaggccgaaaatttagaggaaaatgaagagcccttcgttgcccccttaggattgagcgtcccgtctgacgtggagttgccaccaaccgctaaaatgcacgccatcatcgagcgcacggccagcttcgtgtgcaggcagggagcacagtttgagatcatgctgaaggccaagcaggcccggaactcccagtttgactttctgcgcttcgaccactacctcaacccctactataagttcatccagaaagccatgaaagagggacgctacactgtcctggcagaaaacaaaagtgacgagaaaaaaaaatcaggagtcagctctgacaatgaagatgatgatgatgaagaagatgggaattaccttcatccctctctctttgcctccaagaagtgtaaccgccttgaagagctgatgaagcccttgaaggtagtggacccagatcatcccctcgcagcacttgttcgtaaggcacaggctgacagttccactcccaccccacacaacgcagacggtgcgcctgtgcagccctcccaggtggagtacacggcagactcgaccgtggcagccatgtattacagctactacatgctaccggacggcacttactgcctggcgccgccccctcccggaatcgacgtgactacttactacagcacccttcctgctggcgtgaccgtgtctaactcccctggagtgacgaccaccgccccaccacctcctgggaccacaccactaccgcccccaaccacagcagagactagcagcggggccacctccacaaccaccaccacaagtgcacttgcccccgtggccgccatcatccccccgccccccgacgtccagcccgtgattgacaagctggccgagtatgtcgccaggaacggcctgaagttcgagaccagtgttcgtgccaagaatgatcaaagatttgagttcctgcagccgtggcaccagtataatgcttattatgagtttaagaagcagttcttcctccagaaagaagggggcgatagcatgcaggctgtgtctgcaccagaagaggctcccacagactctgctcccgagaagccaagtgatgctggggaggatggcgcgcctgaagacgcagccgaggtgggagcacgggcaggctcaggcgggaagaaggaggcatcgtccagtaagaccgtcccggacgggaagctggtgaaagcttcctttgctccaataagctttgcaatcaaggccaaagaaaatgatctgcttcccctggaaaaaaatcgtgttaagctagatgatgacagtgatgatgatgaagaaagcaaagaaggccaagaaagttctagtagtgctgcaaacactaacccagcagttgccccaccctgtgtagttgttgaggagaagaagcctcaacttacccaggaggagctagaagcaaagcaagcaaagcaaaagctggaagatcgcctcgcagctgctgcccgggaaaagctggcccaggcgtctaaggagtcaaaagagaaacagcttcaagcagaacgtaaaaggaaagcggcgttatttttacagaccctcaaaaatcctctgccggaagcagaagctgggaaaattgaggagagtcctttcagtgtcgaggaatccagcactacgccctgccctctactgactggaggcaggcctctgcctactttagaagttaaaccacccgataggccttcgagcaaaagcaaagatccaccgagagaagaagagaaagaaaagaaaaagaaaaagcacaaaaaaagatctcgaacaagatcacgttctcccaagtaccattcgtcatccaagtccaggtctagatcacactcaaaagcaaagcattctcttcccagtgcctatcggacagtgcggcggtcgaggtcccgctcccggtcccctcggaggagagcccactcccctgagagacggagggaagagaggagtgtgcccactgcctaccgcgtgagccgcagccctggggccagcaggaagcggacccgctccagaagtccccacgagaagaagaagaagaggcggtcccggtcgcggaccaagtccaaggccaggtctcagtcggtgtcacccagcaagcaggcagcgccccggcccgcggcccccgcggcccactcggcgcactcagccagcgtctcccctgtggagagtcggggctccagccaggagcgctccaggggagtctctcaggaaaaagaagcccagatctcttcagcaatcgtttcttccgtgcagagcaaaatcactcaggatctcatggccaaagtcagagcgatgcttgcagcttccaaaaacctgcaaaccagcgcttcctgagacggggccagcggaggcagagccgggaggctgcgtgggcttctgggcaggctcacgcagacgccggccacaccatccacctggccgcctccatggacccttggtggcttttgtaaattaatttttgatgacattttgagttttaagatttctgaccagcagtctcttacctgtatatttgtaaatatatcatgtttctgtgaaaatgtattatgaaataaaatgggaggaaacaccttttctagctag

SEQ ID NO: 19 - DNA, 20 bp, Artificial Sequence
  forward PCR primer
tgatctctaagaccctttcc

SEQ ID NO: 20 - DNA, 20 bp, Artificial Sequence
  reversed PCR primer
caggttatcatgatcagctc

SEQ ID NO: 21 - DNA, 20 bp, Artificial Sequence
  primers for the single base extension detection method
tctatgctagtgtgacactt

SEQ ID NO: 22 - DNA, 19 bp, Artificial Sequence
  forward PCR primer
aagtgcctggcttcttgag

SEQ ID NO: 23 - DNA, 20 bp, Artificial Sequence
  reversed PCR primer
aaggaagagtgaccaaacac

SEQ ID NO: 24 - DNA, 20 bp, Artificial Sequence
  primers for the single base extension detection method
gccagggagagaaacagcac

SEQ ID NO: 25 - DNA, 19 bp, Artificial Sequence
  forward PCR primer
aagtgcctggcttcttgag

SEQ ID NO: 26 - DNA, 20 bp, Artificial Sequence
  reversed PCR primer
aaggaagagtgaccaaacac

SEQ ID NO: 27 - DNA, 20 bp, Artificial Sequence
  primers for the single base extension detection method
cccttgggatccatcagcca

SEQ ID NO: 28 - DNA, 19 bp, Artificial Sequence
  forward PCR primer
aagtgcctggcttcttgag

SEQ ID NO: 29 - DNA, 20 bp, Artificial Sequence
  reversed PCR primer
aaggaagagtgaccaaacac

SEQ ID NO: 30 - DNA, 20 bp, Artificial Sequence
  primers for the single base extension detection method
tgtgagcagctgccaggctc

SEQ ID NO: 31 - DNA, 21 bp, Artificial Sequence
  forward PCR primer
agttatctaagttcagctgtg

SEQ ID NO: 32 - DNA, 19 bp, Artificial Sequence
  reversed PCR primer
cagaagcaagcttcgtgtc

SEQ ID NO: 33 - DNA, 20 bp, Artificial Sequence
  primers for the single base extension detection method
gggggtgtgtagtcacctcg

SEQ ID NO: 34 - DNA, 21 bp, Artificial Sequence
  forward PCR primer
agttatctaagttcagctgtg

SEQ ID NO: 35 - DNA, 19 bp, Artificial Sequence
  reversed PCR primer
cagaagcaagcttcgtgtc

SEQ ID NO: 36 - DNA, 20 bp, Artificial Sequence
  primers for the single base extension detection method
cggctttgggcagaaacatg

SEQ ID NO: 37 - DNA, 20 bp, Artificial Sequence
  forward PCR primer
ataccaatctgtgcactgac

SEQ ID NO: 38 - DNA, 19 bp, Artificial Sequence
  reversed PCR primer
gttgacccgcaaaaggaag

SEQ ID NO: 39 - DNA, 20 bp, Artificial Sequence
  primers for the single base extension detection method
atgttaactgaagttacttc

SEQ ID NO: 40 - DNA, 20 bp, Artificial Sequence
  forward PCR primer
tgtaacactccaatactgcc

SEQ ID NO: 41 - DNA, 20 bp, Artificial Sequence
  reversed PCR primer
tatccatagacggcagtatc

SEQ ID NO: 42 - DNA, 20 bp, Artificial Sequence
  primers for the single base extension detection method
ctttctcaacccatgtctta

SEQ ID NO: 43 - DNA, 20 bp, Artificial Sequence
  forward PCR primer
tgaaggtcttctccatgatg

SEQ ID NO: 44 - DNA, 21 bp, Artificial Sequence
  reversed PCR primer
tctggtaattgccaaatgatg

SEQ ID NO: 45 - DNA, 20 bp, Artificial Sequence
  primers for the single base extension detection method
taatcagatagtacagtaat

SEQ ID NO: 46 - DNA, 20 bp, Artificial Sequence
  forward PCR primer
catcttgtcttctaagaggc

SEQ ID NO: 47 - DNA, 20 bp, Artificial Sequence
  reversed PCR primer
catacaactccagtctgatg

SEQ ID NO: 48 - DNA, 20 bp, Artificial Sequence
  primers for the single base extension detection method
agggagtgagccataactgg

SEQ ID NO: 49 - DNA, 20 bp, Artificial Sequence
  forward PCR primer
acagtatgtatctgggttgc

SEQ ID NO: 50 - DNA, 21 bp, Artificial Sequence
  reversed PCR primer
ttgaagttctcattgcacaag

SEQ ID NO: 51 - DNA, 18 bp, Artificial Sequence
  primers for the single base extension detection method
actgttgcaatgaacatt

SEQ ID NO: 52 - DNA, 20 bp, Artificial Sequence
  forward PCR primer
gttccctatcactctttaat

SEQ ID NO: 53 - DNA, 24 bp, Artificial Sequence
  reversed PCR primer
tttcatattactttgaattttatt

SEQ ID NO: 54 - DNA, 20 bp, Artificial Sequence
  primers for the single base extension detection method
aaaatcatctgtaaatccag

SEQ ID NO: 55 - DNA, 21 bp, Artificial Sequence
  forward PCR primer
tgtacatagacattaagagac

SEQ ID NO: 56 - DNA, 21 bp, Artificial Sequence
  reversed PCR primer
agcccacacttaggtgatagc

SEQ ID NO: 57 - DNA, 21 bp, Artificial Sequence
  primers for the single base extension detection method
cacatgttcagtgtagtttta

SEQ ID NO: 58 - DNA, 20 bp, Artificial Sequence
  forward PCR primer
caaaagagaggcagcaaatg

SEQ ID NO: 59 - DNA, 20 bp, Artificial Sequence
  reversed PCR primer
cacagttgcatgtgaaatcg

SEQ ID NO: 60 - DNA, 20 bp, Artificial Sequence
  primers for the single base extension detection method
aatgtggacactgaagagac

SEQ ID NO: 61 - DNA, 20 bp, Artificial Sequence
  forward PCR primer
aaagcctgaaaattctgcgg

SEQ ID NO: 62 - DNA, 20 bp, Artificial Sequence
  reversed PCR primer
tacttagatccaaggtctgc

SEQ ID NO: 63 - DNA, 20 bp, Artificial Sequence
  primers for the single base extension detection method
aactttctacagaagttctg

SEQ ID NO: 64 - DNA, 20 bp, Artificial Sequence
  forward PCR primer
ttgcgatatctggatctcag

SEQ ID NO: 65 - DNA, 20 bp, Artificial Sequence
  reversed PCR primer
tgacttgctgtcatcatcac

SEQ ID NO: 66 - DNA, 20 bp, Artificial Sequence
  primers for the single base extension detection method
gtctggtgggttaaccatac

SEQ ID NO: 67 - DNA, 20 bp, Artificial Sequence
  forward PCR primer
gtcaccattctgcttggttg

SEQ ID NO: 68 - DNA, 20 bp, Artificial Sequence
  reversed PCR primer
acaagtttctgagacagcac

SEQ ID NO: 69 - DNA, 18 bp, Artificial Sequence
  primers for the single base extension detection method
cctcctccagcacctggc

SEQ ID NO: 70 - DNA, 20 bp, Artificial Sequence
  forward PCR primer
tgtgtgtctgatttgggttg

SEQ ID NO: 71 - DNA, 20 bp, Artificial Sequence
  reversed PCR primer
ttctaggctcacaccatttg

SEQ ID NO: 72 - DNA, 20 bp, Artificial Sequence
  primers for the single base extension detection method
cttctgtaaaacacacgcta

SEQ ID NO: 73 - DNA, 20 bp, Artificial Sequence
  forward PCR primer
tgtgtgtctgatttgggttg

SEQ ID NO: 74 - DNA, 20 bp, Artificial Sequence
  reversed PCR primer
ttctaggctcacaccatttg

SEQ ID NO: 75 - DNA, 20 bp, Artificial Sequence
  primers for the single base extension detection method
aaaattagaacaacagaaac

SEQ ID NO: 76 - DNA, 21 bp, Artificial Sequence
  forward PCR primer
cattctggacctaatctgatg

SEQ ID NO: 77 - DNA, 21 bp, Artificial Sequence
  reversed PCR primer
tatcagacaggtctagttctg

SEQ ID NO: 78 - DNA, 20 bp, Artificial Sequence
  primers for the single base extension detection method
caggaaaatgcaggtcagca

SEQ ID NO: 79 - DNA, 20 bp, Artificial Sequence
  forward PCR primer
atgtgacagaactagacctg

SEQ ID NO: 80 - DNA, 21 bp, Artificial Sequence
  reversed PCR primer
tataagtcttgaaatgccctc

SEQ ID NO: 81 - DNA, 20 bp, Artificial Sequence
  primers for the single base extension detection method
aatggcttgaatatcacaga

SEQ ID NO: 82 - DNA, 20 bp, Artificial Sequence
  forward PCR primer
ctatttcagattagcaggcg

SEQ ID NO: 83 - DNA, 20 bp, Artificial Sequence
  reversed PCR primer
aaactgctggagtaatgtcc

SEQ ID NO: 84 - DNA, 20 bp, Artificial Sequence
  primers for the single base extension detection method
gatttatcccttaataggct

SEQ ID NO: 85 - DNA, 20 bp, Artificial Sequence
  forward PCR primer
aattgctcatggccagaaac

SEQ ID NO: 86 - DNA, 20 bp, Artificial Sequence
  reversed PCR primer
agggtattcacaggtgtatg

SEQ ID NO: 87 - DNA, 20 bp, Artificial Sequence
  primers for the single base extension detection method
ggccttacgagaactaaata

SEQ ID NO: 88 - DNA, 20 bp, Artificial Sequence
  forward PCR primer
ggagcatgtacatttcagag

SEQ ID NO: 89 - DNA, 20 bp, Artificial Sequence
  reversed PCR primer
acctgaagacagaatcagac

SEQ ID NO: 90 - DNA, 20 bp, Artificial Sequence
  primers for the single base extension detection method
gaaaactctcattttgaatg

SEQ ID NO: 91 - DNA, 20 bp, Artificial Sequence
  forward PCR primer
ggagcatgtacatttcagag

SEQ ID NO: 92 - DNA, 20 bp, Artificial Sequence
  reversed PCR primer
acctgaagacagaatcagac

SEQ ID NO: 93 - DNA, 20 bp, Artificial Sequence
  primers for the single base extension detection method
tttcaagtgaggcagttgga

SEQ ID NO: 94 - DNA, 20 bp, Artificial Sequence
  forward PCR primer
ggagcatgtacatttcagag

SEQ ID NO: 95 - DNA, 20 bp, Artificial Sequence
  reversed PCR primer
acctgaagacagaatcagac

SEQ ID NO: 96 - DNA, 20 bp, Artificial Sequence
  primers for the single base extension detection method
atgccacacatgcttttccc

SEQ ID NO: 97 - DNA, 20 bp, Artificial Sequence
  forward PCR primer
ctgcccatcttaaacacaac

SEQ ID NO: 98 - DNA, 21 bp, Artificial Sequence
  reversed PCR primer
attgtcaggttttctatgtcc

SEQ ID NO: 99 - DNA, 20 bp, Artificial Sequence
  primers for the single base extension detection method
aacgaaatcttagtttagaa

SEQ ID NO: 100 - DNA, 20 bp, Artificial Sequence
  forward PCR primer
aaccttactccaacctcttg

SEQ ID NO: 101 - DNA, 20 bp, Artificial Sequence
  reversed PCR primer
gagatccagctgttgaattc

SEQ ID NO: 102 - DNA, 20 bp, Artificial Sequence
  primers for the single base extension detection method
catcattcatatgaggaaat

SEQ ID NO: 103 - DNA, 20 bp, Artificial Sequence
  forward PCR primer
gtttctggcagaataggtac

SEQ ID NO: 104 - DNA, 20 bp, Artificial Sequence
  reversed PCR primer
agataggcatggtgttagtc

SEQ ID NO: 105 - DNA, 20 bp, Artificial Sequence
  primers for the single base extension detection method
tcccaaagtcctcagaattc

SEQ ID NO: 106 - DNA, 20 bp, Artificial Sequence
  forward PCR primer
gtttctggcagaataggtac

SEQ ID NO: 107 - DNA, 20 bp, Artificial Sequence
  reversed PCR primer
agataggcatggtgttagtc

SEQ ID NO: 108 - DNA, 18 bp, Artificial Sequence
  primers for the single base extension detection method
caactacctctgttctac

SEQ ID NO: 109 - DNA, 20 bp, Artificial Sequence
  forward PCR primer
tgctattccctcctagatac

SEQ ID NO: 110 - DNA, 20 bp, Artificial Sequence
  reversed PCR primer
ttggatgatctgccttaagc

SEQ ID NO: 111 - DNA, 21 bp, Artificial Sequence
  forward PCR primer
gaccgtggcagccatgtatta

SEQ ID NO: 112 - DNA, 19 bp, Artificial Sequence
  reversed PCR primer
ggtcgtcactccaggggag

SEQ ID NO: 113 - DNA, 27 bp, Artificial Sequence
  TAQMAN probes
ccctcccggaatcgacgtgacttamra

SEQ ID NO: 114 - DNA, 28 bp, Artificial Sequence
  TAQMAN probes
cccctcccggaatcgatgtgacttamra

SEQ ID NO: 115 - DNA, 18 bp, Artificial Sequence
  forward PCR primer
ctggaagatcgcctcgca

SEQ ID NO: 116 - DNA, 19 bp, Artificial Sequence
  reversed PCR primer
tctgcttccggcagaggat

SEQ ID NO: 117 - DNA, 24 bp, Artificial Sequence
  TAQMAN probes
tgcccgggaaaagctggcctamra

SEQ ID NO: 118 - DNA, 24 bp, Artificial Sequence
  TAQMAN probes
tgcccgggaaaagctcgcctamra

SEQ ID NO: 119 - DNA, 16 bp, Artificial Sequence
  forward PCR primer
cgccaccctgggcaga

SEQ ID NO: 120 - DNA, 18 bp, Artificial Sequence
  reversed PCR primer
tgctgcagcctgccacat

SEQ ID NO: 121 - DNA, 25 bp, Artificial Sequence
  TAQMAN probes
cctccgcgtccctcaccatgtamra

SEQ ID NO: 122 - DNA, 23 bp, Artificial Sequence
  TAQMAN probes
agcctccgcgcccctcactamra

SEQ ID NO: 123 - DNA, 21 bp, Artificial Sequence
  forward PCR primer
tgagtcaaaccatgtcctgcc

SEQ ID NO: 124 - DNA, 22 bp, Artificial Sequence
  reversed PCR primer
cgtggtgtccatgttagtggag

SEQ ID NO: 125 - DNA, 18 bp, Artificial Sequence
  TAQMAN probes
gcctagtcactaaaamgb

SEQ ID NO: 126 - DNA, 19 bp, Artificial Sequence
  TAQMAN probes
gcctagtcactagaacmgb

(19) United States
(12) Patent Application Publication
(10) Pub. No.: US 2009/0297563 A1
(43) Pub. Date: Dec. 3, 2009

(54) Diagnosis And Treatment of Immune-Related Diseases

(75) Inventors: Anders Borglum, Arhus C (DK); Torben Kruse, Svendborg (DK); Annette Haagerup, Ry (DK); Charlotte Brasch Andersen, Valby (DK)

Correspondence Address: GIFFORD, KRASS, SPRINKLE,ANDERSON & CITKOWSKI, P.C, PO BOX 7021, TROY, MI, 48007-7021, US

(73) Assignee: Anders Borglum, Arhus C (DK)

(21) Appl. No.: 12/067,443

(22) PCT Filed: Oct. 27, 2005
(86) PCT No.: PCT/DK2005/000693

§ 371 Date: Aug. 27, 2008

(30) Foreign Application Priority Data
Oct. 27, 2004 (DK) PA 2004 01646
Oct. 27, 2004 (DK) PA 2004 01647
Sep. 1, 2005 (DK) PA 2005 01215

(51) Int. Cl. A61K 39/35; C12Q 1/68; C12N 15/63; A61K 31/7088; C07K 14/00; C07K 16/00; C07H 21/04; C07H 21/02; C07H 21/00

(52) U.S. Cl. 424/275.1; 435/6; 435/320.1; 514/44 R; 514/44 A; 530/350; 530/387.9; 536/23.1; 536/24.5

(57) ABSTRACT

The present invention relates to association of one or more polymorphisms located in the human SFRS8, CD83, SLAMF1, CD86, HRH1, IL2, TLR7, TLR8 and TLR10 genes to the occurrence of allergic diseases such as rhinitis, asthma, and atopic dermatitis, auto-immune diseases, infectious diseases, and graft/host incompatibilities. The invention relates both to methods for diagnosing a predisposition to said diseases, classifying said diseases and to methods and compositions for treating subjects with said diseases. Furthermore the invention relates to screens for identifying compounds effective in treating said diseases. The invention describes specific single nucleotide polymorphisms the presence of which in the genome of an individual is strongly associated with the predisposition of said individual to an immune related disease.

### FIELD OF INVENTION

The present invention relates to association of one or more polymorphisms located in the human SFRS8, CD83, SLAMF1, CD86, HRH1, IL2, TLR7, TLR8 and TLR10 genes to the occurrence of allergic diseases such as rhinitis, asthma, and atopic dermatitis, auto-immune diseases, infectious diseases, and graft/host incompatibilities. The invention relates both to methods for diagnosing a predisposition to said diseases, classifying said diseases and to methods and compositions for treating subjects with said diseases. Furthermore the invention relates to screens for identifying compounds effective in treating said diseases.

### BACKGROUND OF INVENTION

### Polymorphisms

DNA polymorphisms provide an efficient way to study the association of genes and diseases by analysis of linkage and linkage disequilibrium. With the sequencing of the human genome a myriad of hitherto unknown genetic polymorphisms among people have been detected. Most common among these are the single nucleotide polymorphisms, also called SNPs, of which we now know several millions. Other examples are variable number of tandem repeat polymorphisms, insertions, deletions and block modifications. Tandem repeats often have multiple different alleles (variants), whereas the other groups of polymorphisms usually have just two alleles. Some of these genetic polymorphisms probably play a direct role in the biology of the individuals, including their risk of developing disease, but the virtue of the majority is that they can serve as markers for the surrounding DNA.

The association of an allele of one sequence polymorphism with particular alleles of other sequence polymorphisms in the surrounding DNA has two origins, known in the genetic field as linkage and linkage disequilibrium, respectively. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the cells of the germline, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less that 10000 bases in the DNA will have tended to stay together since modern man arose. Linkage disequilbrium in limited populations, for instance Europeans, often extends over longer distances, e.g. over more than 1,000,000 bases. This can be the result of newer mutations, but can also be a consequence of one or more “bottlenecks” with small population sizes and considerable inbreeding in the history of the current population. Two obvious possibilities for “bottlenecks” in Europeans are the exodus from Africa and the repopulation of Europe after the last ice age.

### Genes

### SFRS8

The human SFRS8 gene has been mapped to chromosome 12q24. The gene encodes a 951-amino acid polypeptide containing putative nuclear localization sequences, an arginine- and serine-rich (R/S) domain, and 2 repeated modules, known as surp modules, which are homologous to regions in the constitutive splicing factor SPP91/PRP21. Denhez and Lafyatis (1994) found that the SFRS8 mRNAs are alternatively spliced, showing that SFRS8 expression is regulated, presumably autogeneously, by control of splicing of the first 2 introns. Sarkissian et al. (1996) demonstrated that SFRS8 protein not only regulates its own splicing but also the splicing of fibronectin and CD45.

CD45, which is also known as T200 glycoprotein or leukocyte-common antigen (LCA), is a major high molecular weight leukocyte cell surface protein tyrosine phosphotase receptor-like molecule. The receptor is essential for the activation of T and B cells by mediating cell-to-cell contacts and regulating protein-tyrosine kinases involved in signal transduction. CD45 is also involved in integrin-mediated adhesion and migration of immune cells.

The CD45 gene contains 35 exons. The CD45 protein exists in multiple isoforms, depending on alternative splicing of exons 4, 5, and 6. The corresponding protein domains are characterized by the binding of monoclonal antibodies specific for CD45RA (exon 4), CD45RB (exon 5), CD45RC (exon 6), and CD45RO (exons 4 to 6 spliced out). In T cells, the alternative splicing of CD45 is regulated so that naive or unprimed T cells predominantly express CD45RA-positive isoforms and switch to expression of CD45RO upon activation. CD45RO expression is correlated with the memory T-cell phenotype (Akbar et al., 1988). Mice and humans lacking CD45 expression are characterized by a block of T-cell maturation (Kishihara et al., 1993; Kung et al., 2000). Among other important functions of CD45 in immune cells is the ability of the protein to suppress JAK kinases (Irie-Sasaki et al., 2001) and down regulate cytokine receptor signaling. Targeted disruption of the CD45 gene has been shown to result in the enhanced cytokine and interferon receptor-mediated activation of JAKs and STAT proteins.

### CD83

The human CD83 gene has been mapped to chromosome 6p23 (Olavesen, et al. 1997).

Using differential hybridization with labeled cDNAs from B- and T-cell lines to screen a human tonsil cDNA library, Zhou et al. (1992) isolated a full-length cDNA clone for CD83, which they termed HB15. The predicted 205-amino acid protein contains 6 cysteine residues in the extracellular region and 1 in the membrane-spanning domain. A pair of cysteine residues are in positions to permit the disulfide bonding that delineates an Ig-like domain. Using flow cytometry on B and T cell lines, CD83 was expressed variably on cells that were proliferating maximally but not on circulating peripheral blood lymphocytes or monocytes. By immunohistologic analysis, Zhou et al. (1992) observed CD83 expression in lymph nodes, spleen, and tonsils and high expression on scattered interfollicular cells. Expression was also noted on a subpopulation of dendritic cells in the epidermis.

Using subtractive cDNA cloning, Koziow et al. (1993) isolated a cDNA clone, BL11, that is expressed selectively or exclusively on activated B lymphocytes. BL11 is identical to CD83.

Zhou and Tedder (1995) found by FACS analysis that CD83 is strongly expressed on a phenotypically homogeneous subpopulation of plastic nonadherent peripheralblood cells that express high levels of MHC class II molecules and are morphologically identical to antigen-presenting dendritic cells.

Berchtold et al. (1999) cloned a cDNA from a mouse bone marrow-derived dendritic cell (BM-DC) cDNA library. The cDNA encodes a 196-amino acid protein that has 63% amino acid identity with human CD83 and contains a 21-amino acid signal sequence. Northern blot analysis revealed strong expression in BM-DC that was upregulated following stimulation by lipopolysaccharide or TNFα. They also showed that CD83 is glycosylated when expressed in COS cells.

It has also been shown that
1. 20% of chronic lymphocytic leukemia & 5/7 mantle-cell lymphoma patients have significantly elevated levels of soluble CD83. sCD83 may have an immunoregulatory role in vivo & functional significance in hematological malignancies, like CLL and MCL;
2. induction of the CD83 promoter by LMP1 of Epstein-barr virus is mediated by the activation of NF-kappaB signal pathway in B cells;
3. Increased expression of DC-SIGN+IL-12+IL-18+ and CD83+IL-12-IL-18-dendritic cell populations in the colonic mucosa of patients with Crohn's disease;
4. the soluble extracellular CD83 domain inhibits DC-mediated T-cell proliferation.

### SLAMF1

Cocks et al. (1995) found that SLAM is constitutively expressed on peripheral blood memory T cells, T-cell clones, immature thymocytes, and a proportion of B cells, and is rapidly induced on naive T cells after activation.

Punnonen et al. (1997) found that activated B cells express the membrane-bound form of SLAM and the soluble and cytoplasmic isoforms of SLAM, and that the expression levels of membrane-bound SLAM on B cells are rapidly regulated after activation in vitro. They presented data suggesting that signaling through homophilic SLAM-SLAM binding during B-B and B-T cell interactions enhances the expansion and differentiation of activated B cells.

The expression of SLAM in rheumatoid arthritis was studied by Isomaki et al. (1997) and in acute multiple sclerosis by Ferrante et al. (1998).

Tatsuo et al. (2000) found that in MV-resistant cell lines infection with clinical MV and expression of SLAM, but not CD46, caused cytopathic effects (CPE). Likewise, anti-SLAM antibody protected cells from CPE when challenged with MV. Lymphoid cell lines expressing SLAM, but not lymphoid and myelomonocytic cell lines devoid of SLAM, were shown to be susceptible to MV. Tatsuo et al. (2000) noted that the expression of SLAM on activated B and T lymphocytes correlates with the pathology of MV infection in humans and monkeys, in which lymphoid organs are the chief sites of MV replication. They proposed that binding of MV to SLAM may impair the signaling functions of SLAM in lymphocyte activation and inhibit Th0/Th1 cytokine production, thereby promoting Th2 cytokine production.

Latour et al. (2001) reported that antibody-mediated ligation of SLAM on thymocytes triggered a protein tyrosine phosphorylation signal in T cells in a SAP-dependent manner. This signal also involved SHIP; the adaptor molecules DOK2, DOK1, and SHC; and RASGAP. SAP was crucial for this pathway because it selectively recruited and activated the T-cell isoform of FYN.

It has also been shown that

1. SLAM mRNA expression in PBMC is modulated during the course of specific immunotherapy, and an early and transient increase of SLAM mRNA expression is associated with clinical symptom improvement;
2. direct correlation between the amount of hSLAM expressed on the cells' surface and the degree of measles virus infection; MV infection induced downregulation of receptor hSLAM and inhibited cell division and proliferation of hSLAM(+)T cells;
3. SLAM expression correlates directly with T cell responsiveness to Mycobacterium tuberculosis antigen;
4. effect of X-linked lymphoproliferative syndrome gene product SAP/SH2D1A on signaling through signaling lymphocyte activation molecule family of immune receptors;
5. susceptibility of human dendritic cells (DCs) to measles virus (MV) depends on their activation stages in conjunction with the level of CDw150: role of Toll stimulators in DC maturation and MV amplification;
6. SLAM contributes to the enhanced immunostimulatory functions of dendritic cells that are observed following the addition of IL-1 in vitro.

### HRH1

Le Coniat et al. (1994) assigned the human histamine H1-receptor gene to chromosome 3 by Southern blot analysis of human/hamster somatic cell hybrids. The assignment was confirmed and refined to 3p21-p14 by isotopic in situ hybridization. Inoue et al. (1996) concluded that the mouse histamine H1 receptor gene (Hrh1) is a single locus and is located in the central portion of mouse chromosome 6 in a region of homology with human chromosome 3p.

The HRH1 gene encodes a G protein-coupled receptor that mediates diverse neuronal and peripheral actions of histamine. Histamine is a ubiquitous messenger molecule released from mast cells, enterochromaffin-like cells, and neurons. Its various actions are mediated by 3 pharmacologically defined receptors termed the H1, H2, and H3 receptors. The H1 receptor was the first member of this family to be pharmacologically defined with the design of selective antagonists, the ‘antihistamines,’ which are used to treat allergic and inflammatory reactions. The H1 receptor is expressed by various peripheral tissues, such as smooth muscle, and by neurons in the brain, where histamine may be involved in the control of wakefulness, mood, and hormone secretion. Yamashita et al. (1991) cloned a bovine H1 receptor cDNA and established its nucleotide sequence. Its homology with the corresponding sequence of other receptors confirmed that it belongs to the superfamily of receptors coupled with G proteins with 7 putative transmembrane domains.

In addition to their expression in neuronal, gastric, and muscular tissue, the G protein-coupled receptors HRH1 and HRH2 are also expressed on T-helper lymphocytes and trigger different intracellular events upon activation. Using flow cytometric analysis, Jutel et al. (2001) demonstrated that histamine binds more strongly to Th1 than to Th2 cells.

Flow cytometry and RT-PCR analysis showed that HRH1 is predominantly expressed on Th1 cells in an IL3-upregulatable manner, while HRH2 is predominant on Th2 cells. Stimulation of naive, CD45RA+ T cells with IL12 resulted in preferential expression of HRH1, but stimulation with IL4 resulted in suppressed expression of HRH1, demonstrating that mature CD45RO+ Th1 and Th2 lymphocytes preferentially but not exclusively express HRH1 and HRH2, and that HRH1 and HRH2 are regulated by cytokines present in the immune environment. Histamine stimulation of Th1 cells resulted in significant calcium flux that could be blocked by an HRH1 antagonist, while stimulation of Th2 cells led to cAMP formation that could be blocked by an HRH2, but not an HRH1, antagonist. Furthermore, histamine enhanced Th1 but inhibited Th2 responses to anti-CD3. Histamine also enhanced peripheral blood mononuclear cell responses in sensitized individuals to a predominantly Th1 antigen, but suppressed responses to Th2 allergens.

Jutel et al. (2001) noted that HRH1 or HRH2 deletions are reported to result in abnormalities in the central nervous and gastrointestinal systems. Mice lacking Hrh1 have lower, whereas Hrh2-deficient mice have higher, percentages of Ifngproducing cells, compared to wildtype mice. Mice lacking either receptor tended to have a higher frequency of 114-producing cells. Hrh1-deficient mice produced higher levels of antigen-specific IgG1 and IgE compared to wildtype mice, whereas levels of these immunoglobulins are reduced in Hrh2 knockout mice, indicating that Ifngmediated suppression of IgE production predominated over the enhancement otherwise seen with enhanced IL4 or IL13 production. Jutel et al. (2001) concluded that histamine secreted from inflammation effector cells potently influences Th1 and Th2 responses as well as antibody isotypes as a regulatory loop in inflammatory reactions.

### TLR7

Toll-like receptors (TLRs), such as TLR7, are a critical part of the evolutionarily conserved innate immune system. TLRs have specificity for different bacterial components, such as lipopolysaccharide (TLR4), bacterial lipoproteins (TLR2), and unmethylated CpG dinucleotides (TLR9).

By genomic sequence analysis, Chuang and Ulevitch (2000) and Du et al. (2000) determined that the TLR7 gene contains 3 exons. However, only the initiator methionine is encoded on exon 2, and the remainder of the protein is encoded on exon 3. Du et al. (2000) stated that the TLR7 gene spans approximately 23 kb.

By genomic DNA database searching for open reading frames with homology to the cytoplasmic domain of TLR4, followed by 5-prime RACE and PCR on a placenta cDNA library, Chuang and Ulevitch (2000) and Du et al. (2000) obtained cDNAs encoding TLR7, TLR8, and TLR9. Sequence analysis predicted that the 1,049-amino acid TLR7 type I transmembrane protein has a signal peptide, multiple leucine-rich repeats (LRRs) and a cysteine-rich region in its extracellular domain.

Its cytoplasmic domain has the characteristic TLR-IL1R (TIR) sequences found in this family of proteins. By PCR on cDNA libraries, Chuang and Ulevitch (2000) detected predominant expression of TLR7 in lung, placenta, and spleen, with lower expression in lymph node and tonsil. By RT-PCR analysis, Du et al. (2000) found expression in lung, brain, spleen, small intestine, and stomach.

Using RT-PCR and ELISA analysis, Kadowaki et al. (2001) defined the differential expression of TLR1 through TLR10 and the pathogen-associated molecular pattern recognition profiles and cytokine production patterns of monocytes and dendritic cell precursors. They concluded that neither monocytes nor dendritic cell precursors can respond to all microbial antigens and that they have limited functional plasticity.

Using luciferase analysis, Chuang and Ulevitch (2000) showed that expression of a chimeric TLR7 containing its transmembrane and cytoplasmic domains, but not overexpression of full-length TLR7, activated nuclear factor kappa-B (NFKB).

Imidazoquinolines are potent synthetic activators of immune cells with antiviral and antitumor properties. Using macrophages from wildtype and Myd88-deficient mice, Hemmi et al. (2002) showed that 2 imidazoquinolines, imiquimod and resiquimod, which are active against genital warts and genital herpes,

respectively, induce tumor necrosis factor (TNF) and interleukin-12 (IL12) cytokines and activate NFKB only in wildtype cells, implying that the activation is through a TLR. Macrophages from mice deficient in Tlr7 but not other Tlrs produced no detectable cytokines in response to these imidazoquinolines. In addition, the imidazoquinolines induced dose-dependent proliferation of splenic B cells and the activation of intracellular signaling cascades in cells from wildtype but not Tlr7−/− mice. Luciferase analysis established that expression of human TLR7, but not TLR2 or TLR4, in human embryonic kidney cells results in NFKB activation in response to resiquimod. Injection of this compound into wildtype but not Tlr7−/− mice induced increased serum concentration of cytokines. Hemmi et al. (2002) concluded that TLR7 is required for imidazoquinoline-induced immune responses and signal cascade activation. They suggested that viral products may themselves activate TLR7 or that viral infection may generate an endogenous ligand that interacts with TLR7 in a manner analogous to that seen in Drosophila.

Using luciferase analysis, Lee et al. (2003) showed that a number of antiviral guanine analogs that induce NFKB activation, cytokine production, and expression of costimulatory molecules do so through stimulation of TLR7, but not other TLRs, in an endosomal acidification-dependent manner.

Diebold et al. (2004) confirmed that mouse plasmacytoid dendritic cells (PDCs) expressing B220 (PTPRC) but not Cd11b (ITGAM) were resistant to suppression of Ifna production mediated by influenza virus NS1 protein, suggesting that PDCs use a dsRNA-independent pathway for recognizing influenza. Chloroquine inhibited influenza-induced Ifna production, indicating that recognition of the virus occurs in the endosomal compartment. Ifna production in response to live or inactivated influenza virus or to viral genomic or host ssRNA required the presence of Myd88 and Tlr7, but not other TLRs.

Heil et al. (2004) showed that GU nucleosides, but not other nucleoside combinations, and the GU-rich sequence from the U5 region of HIV-1 induced TNF, IFNα, IL12p40, and IL6 production by CD123 (IL3RA)-positive or BDCA4-positive PDCs. Mouse DCs deficient in Tlr7, but not those deficient in Tlr3 or Tlr9, were unable to respond to GU-rich ssRNA. In contrast, TLR8 was required for responsiveness to ssRNA in transfected human cells, supporting the observation of species-specific differences for TLR7 and TLR8. Heil et al. (2004) concluded that single-stranded GU-rich RNA is a natural ligand for mouse Tlr7 and human TLR8. They proposed that recognition occurs in endosomal or lysosomal compartments, because Tlr7 and TLR8 signaling requires acidification of these compartments.

### TLR8

By genomic sequence analysis, Chuang and Ulevitch (2000) determined that the TLR8 gene contains 2 exons, with the initiator methionine encoded on exon 1, and the remainder of the protein encoded on exon 2. However, Du et al. (2000) stated that the gene spans approximately 15.5 kb and contains 3 exons, with exon 3 being the major coding exon. Chuang and Ulevitch (2000) and Du et al. (2000) mapped the TLR8 gene to Xp22.3-p22.2, approximately 16 kb telomeric to the TLR7 gene.

The protein encoded by this gene is a member of the Toll-like receptor (TLR) family which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity. The various TLRs exhibit different patterns of expression. This gene is predominantly expressed in lung and peripheral blood leukocytes, and lies in close proximity to another family member, TLR7, on chromosome X.

Heil et al. (2004) showed that GU nucleosides, but not other nucleoside combinations, and the GU-rich sequence from the U5 region of HIV-1 induced TNF, IFNα, IL12p40, and IL6 production by CD123-positive or BDCA4-positive plasmacytoid dendritic cells (PDCs).

Mouse DCs deficient in Tlr7, but not those deficient in Tlr3 or Tlr9, were unable to respond to GU-rich ssRNA. In contrast, TLR8 was required for responsiveness to ssRNA in transfected human cells, supporting the observation of species-specific differences for TLR7 and TLR8. Heil et al. (2004) concluded that single-stranded GU-rich RNA is a natural ligand for mouse Tlr7 and human TLR8. They proposed that recognition occurs in endosomal or lysosomal compartments, because Tlr7 and TLR8 signaling requires acidification of these compartments.

### TLR10

By searching DNA and EST databases, followed by 5-prime RACE and PCR on a spleen cDNA library, Chuang and Ulevitch (2001) isolated a cDNA encoding TLR10. Sequence analysis predicted that the 811-amino acid protein, which is approximately 50% identical to TLR1 and TLR6, contains a signal peptide, multiple leucine-rich repeats, a cysteine-rich domain, a transmembrane domain, and a cytoplasmic TIR domain. RT-PCR analysis detected expression of TLR10 predominantly in immune cell-rich tissues, such as spleen, lymph node, thymus, and tonsil, as well as in lung. Expression was also detected in immune cell lines, although a T-cell line failed to show expression of TLR10.

Using RT-PCR and ELISA analysis, Kadowaki et al. (2001) defined the differential expression of TLR1 through TLR10 and the pathogen-associated molecular pattern recognition profiles and cytokine production patterns of monocytes and dendritic cell precursors. They concluded that neither monocytes nor dendritic cell precursors can respond to all microbial antigens and that they have limited functional plasticity.

### IL2

Interleukin-2 (IL2), formerly referred to as T-cell growth factor, is a powerfull immunoregulatory lymphokine that is produced by lectin- or antigen-activated T cells. Not only is it produced by mature T lymphocytes on stimulation but also constitutively by certain T-cell lymphoma cell lines. It is useful in the study of the molecular nature of T-cell differentiation and because, like interferons, it augments natural killer cell α-tivity, it might have use in the treatment of cancer. Lowenthal et al. (1985) presented evidence that IL2 can act as a growth hormone for both B and T lymphocytes. Thus, IL2 is a better designation than TCGF (See review by Smith (1988). IL2 has a molecular weight of 15,000. Taniguchi et al. (1983) cloned the human IL2 gene. Fujita et al. (1983) found that the IL2 gene has a promoter sequence homologous to that of the human gamma interferon gene.

Using a cloned human TCGF gene in somatic cell hybridization studies, Seigel et al. (1984) assigned the TCGF locus to chromosome 4. In situ hybridization narrowed the assignment to 4q26-q28. Evidence was presented to indicate that TCGF and RAF2 (164760), the pseudogene form of the oncogene RAF1, is not closely linked to TCGF although it is on chromosome 4. Fiorentino et al. (1989) assigned the 112 locus to mouse chromosome 3 by Southern analysis of Chinese hamster/mouse somatic cell hybrid cells, and Webb et al. (1990) localized it to bands B-C by in situ hybridization.

Since interleukin-2 and interleukin-2 receptor act as required for the proliferation of T cells, defects in either the ligand or the receptor would be expected to cause severe combined immunodeficiency. Weinberg and Parkman (1990) described a male Salvadoran infant with severe combined immunodeficiency and a specific absence of IL2 mRNA. The IL2 gene was present, indicating that the defect was not due to a sizable deletion. The infant died following bone marrow transplantation. The use of recombinant interleukin-2 in the treatment of such patients was discussed.

Using fluorescence in situ hybridization and single-cell PCR in cells with different IL2 alleles, Hollander et al. (1998) demonstrated that in mature thymocytes and T cells, IL2 expression is monoallelic. Since IL2 is encoded at a nonimprinted autosomal locus, this result indicated an unusual mechanism for regulating the expression of a single gene.

Memory T cells maintain their numbers for long periods after antigen exposure. Ku et al. (2000) demonstrated that CD8+ T cells of memory phenotype divide slowly in animals. This division requires interleukin-15 (600554) and is markedly increased by inhibition of interleukin-2. The authors therefore suggested that the numbers of CD8+ memory T cells in animals are controlled by a balance between IL15 and IL2.

Yang et al. (2001) analyzed T-cell subsets and levels of cytokine IL2 and soluble IL2 receptor in the peripheral blood of patients with normal pressure glaucoma (NPG) and primary open angle glaucoma (POAG) and compared them to values in agematched controls. They found increased frequency of CD8+/HLA-DR+lymphocytes in patients with NPG and increased CD3+/CD8+ lymphocytes in both NPG and POAG patients. CD5+ lymphocytes were higher only in POAG patients. The mean concentration of soluble IL2R was higher in NPG and POAG patients than in controls although the IL2 concentration was similar in patients and controls. The authors concluded that the immune system might play an important role in initiation or progression of glaucomatous optic neuropathy in some patients.

Helicobacter pylori vacuolating cytotoxin VacA induces cellular vacuolation in epithelial cells. Gebert et al. (2003) found that VacA could efficiently block proliferation of T cells by inducing a G1/S cell cycle arrest. VacA interfered with the T cell receptor/IL2 signaling pathway at the level of the calcium-calmodulin-dependent phosphatase calcineurin. Nuclear translocation of NFAT was abrogated, resulting in downregulation of IL2 transcription. VacA partially mimicked the activity of the immunosuppressive drug FK506 by possibly inducing a local immune suppression, explaining the extraordinary chronicity of Helicobacter pylori infections.

### CD86

Induction of an immune response requires that T cells receive 2 sets of signals from antigen-presenting cells. The first signal is delivered through the T-cell receptor complex, while the second is provided by the B-cell activation antigens B7-1, or CD80, and B7-2, or CD86, by interaction with the T-cell surface molecules, CD28 and CTLA4. A cDNA for B7-2 was obtained by Freeman et al. (1993). B7-2 mRNA is constitutively expressed in unstimulated B cells. The predicted protein is a type I membrane protein of the immunoglobin superfamily.

Jeannin et al. (2000) detected a soluble form of CD86 in human serum that could be generated either by shedding of the membrane form or through alternative splicing. RT-PCR analysis revealed the expression of 2 transcripts in nonstimulated monocytes but only the full-length transmembrane form in activated monocytes. The smallest transcript, 828 bp, which the authors termed CD86delta™, has a deletion from nucleotide 686 to nucleotide 829 (i.e., exon 6) and encodes a 275-amino acid protein. SDS-PAGE and Western blot analysis detected expression of CD86 and CD86delta™ in COS cells as 65- and 48-kD proteins, respectively. FACS analysis detected only CD86 transfected cells and ELISA analysis detected only CD86delta™ in cell-free supernatants. Binding analysis demonstrated that CD86delta™ binds to CD28- or CTLA4-expressing cells. Functional analysis indicated that CD86delta™ enhances proliferation and cytokine production by both naive and memory T cells.

Resting eosinophils express neither MHC class II proteins or co-stimulatory B7 molecules and fail to induce proliferation of T cells to antigens. Celestin et al. (2001) reported that IL3 induces expression of HLA-DR and B7.2 on eosinophils, but, unlike IL5 and GMCSF (CSF2), it does not induce expression of B7.1. IL3-treated eosinophils supported modest T-cell proliferation in response to superantigen toxic shock syndrome-1 antigen, as well as proliferation of HLA-DR-restricted T-cell clones to tetanus toxoid (TT) and influenza virus antigenic peptides. The response was blocked by anti-B7.2 monoclonal antibody. IL3-treated eosinophils were unable to present native TT antigen to either resting or TT-specific cloned T cells. Parallel experiments established that IL5 and GMCSF induce T-cell proliferation to peptides but not to native TT antigen. Celestin et al. (2001) suggested that eosinophils activated by IL3 may contribute to T-cell activation in allergic and parasitic diseases by presenting superantigens and peptides to T cells.

An immune response against thyroid carcinoma could be important for long-term survival. Gupta et al. (2001) reported that infiltration of thyroid carcinoma by proliferating lymphocytes is associated with improved disease-free survival. Shah et al. (2002) hypothesized that the antigen presentation co-activators B71 and B72, which are important in other immune-mediated thyroid diseases, might be important in lymphocytic infiltration of thyroid carcinoma. To test this, they determined B71 and B72 expression by immunohistochemistry in 27 papillary (PTC) and 8 follicular (FTC) thyroid carcinomas and 9 benign thyroid lesions. B72 expression was of similar intensity in benign and malignant tumors, but was more intense than in presumably normal adjacent thyroid. B72 expression also correlated with the number of tumor-associated lymphocytes per high-power field. Recurrence developed exclusively from tumors that expressed B72, and intense B72 expression was associated with a reduced probability of remission. Shah et al. (2002) concluded that these data support the hypothesis that the antigen presentation co-activators B71 and B72 may be important for lymphocytic infiltration and the immune response against thyroid carcinoma.

Jellis et al. (1995) isolated the gene for CD86 (B7-2), which is composed of 8 exons and spans more than 22 kb. The authors found that alternatively spliced cDNAs result from the use of either exon 1 or 2. Exon 3 corresponds to the signal peptide, exon 4 to an IgV-like domain, exon 5 to an IgC-like domain and exon 6 corresponds to the transmembrane region and part of the cytoplasmic tail. Exons 7 and 8 encode the remainder of the tail.

Reeves et al. (1997) demonstrated that the CD86 and CD80 genes are linked on human chromosome 3 and mouse chromosome 16. Reeves et al. (1997) used fluorescence in situ hybridization mapping to show that CD86, like CD80, maps to human 3q21 and mouse chromosome 16, band B5.

### REFERENCES

Akbar, A. N.; Terry, L.; Timms, A.; Beverley, P. C.; Janossy, G. Loss of CD45R and gain of UCHL1 reactivity is a feature of primed T cells. J. Immun. 140: 2171-2178, 1988.
Berchtold, S.; Muhl-Zurbes, P.; Heufler, C.; Winklehner, P.; Schuler, G.; Steinkasserer, A. Cloning, recombinant expression and biochemical characterization of the murine CD83 molecule which is specifically upregulated during dendritic cell maturation. FEBS Lett. 461: 211-216, 1999.
Celestin, J.; Rotschke, O.; Falk, K.; Ramesh, N.; Jabara, H.; Strominger, J.; Geha, R. S. IL-3 induces B7.2 (CD86) expression and costimulatory activity in human eosinophils. J. Immun. 167: 6097-6104, 2001.
Chuang, T.-H.; Ulevitch, R. J. Cloning and characterization of a sub-family of human Toll-like receptors: hTLR7, hTLR8 and hTLR9. Europ. Cytokine Netw. 11: 372-378, 2000.
Cocks, B. G.; Chang, C.-C. J.; Carballido, J. M.; Yssel, H.; de Vries, J. E.; Aversa, G. A novel receptor involved in T-cell activation. Nature 376: 260-263, 1995.
Denhez, F.; Lafyatis, R. Conservation of regulated alternative splicing and identification of functional domains in vertebrate homologs to the Drosophila splicing regulator, suppressor-of-white-apricot. J. Biol. Chem. 269: 16170-16179, 1994.
Diebold, S. S.; Kaisho, T.; Hemmi, H.; Akira, S.; Reis e Sousa, C. Innate antiviral responses by means of TLR7-mediated recognition of single-stranded RNA. Science 303: 1529-1531, 2004.
Du, X.; Poltorak, A.; Wei, Y.; Beutler, B. Three novel mammalian toll-like receptors: gene structure, expression, and evolution. Europ. Cytokine Netw. 11: 362-371, 2000.
Fiorentino, L.; Austen, D.; Pravtcheva, D.; Ruddle, F. H.; Brownell, E. Assignment of the interleukin-2 locus to mouse chromosome 3. Genomics 5: 651-653, 1989.
Freeman, G. J.; Gribben, J. G.; Boussiotis, V. A.; Ng, J. W.; Restivo, V. A.; Lombard, L. A.; Gray, G. S.; Nadler, L. M. Cloning of B7-2: a CTLA-4 counter-receptor that costimulates human T cell proliferation. Science 262: 909-911, 1993.
Fujita, T.; Takaoka, C.; Matsui, H.; Taniguchi, T. Structure of the human interleukin 2 gene. Proc. Nat. Acad. Sci. 80: 7437-7441, 1983.
Gebert, B.; Fischer, W.; Weiss, E.; Hoffmann, R.; Haas, R. Helicobacter pylori vacuolating cytotoxin inhibits T lymphocyte activation. Science 301: 1099-1102, 2003.
Gupta, S.; Patel, A.; Folstad, A.; Fenton, C.; Dinauer, C. A.; Tuttle, R. M.; Conran, R.; Francis, G. L. Infiltration of differentiated thyroid carcinoma by proliferating lymphocytes is associated with improved disease-free survival for children and young adults. J. Clin. Endocr. Metab. 86: 1346-1354, 2001.
Heil, F.; Hemmi, H.; Hochrein, H.; Ampenberger, F.; Kirschning, C.; Akira, S.; Lipford, G.; Wagner, H.; Bauer, S. Species-specific recognition of single-stranded RNA via Toll-like receptor 7 and 8. Science 303: 1526-1529, 2004.
Hemmi, H.; Kaisho, T.; Takeuchi, O.; Sato, S.; Sanjo, H.; Hoshino, K.; Horiuchi, T.; Tomizawa, H.; Takeda, K.; Akira, S. Small anti-viral compounds activate immune cells via the TLR7MyD88-dependent signaling pathway. Nature Immun. 3: 196-200, 2002.
Hollander, G. A.; Zuklys, S.; Morel, C.; Mizoguchi, E.; Mobisson, K.; Simpson, S.; Terhorst, C.; Wishart, W.; Golan, D. E.; Bhan, A. K.; Burakoff, S. J. Monoallelic expression of the interleukin-2 locus. Science 279: 2118-2121, 1998.
Jeannin, P.; Magistrelli, G.; Aubry, J.-P.; Caron, G.; Gauchat, J.-F.; Renno, T.; Herbault, N.; Goetsch, L.; Blaecke, A.; Dietrich, P.-Y.; Bonnefoy, J.-Y.; Delneste, Y. Soluble CD86 is a costimulatory molecule for human T lymphocytes. Immunity 13: 303-312, 2000.
Jellis, C. L.; Wang, S. S.; Rennert, P.; Borriello, F.; Sharpe, A. H.; Green, N. R.; Gray, G. S. Genomic organization of the gene coding for the costimulatory human B-lymphocyte antigen B7-2 (CD86). Immunogenetics 42: 85-89, 1995.
Jutel, M.; Watanabe, T.; Klunker, S.; Akdis, M.; Thomet, 0. A. R.; Malolepszy, J.; Zak-Nejmark, T.; Koga, R.; Kobayashi, T.; Blaser, K.; Akdis, C. A. Histamine regulates T-cell and antibody responses by differential expression of H1 and H2 receptors. Nature 413: 420-425, 2001.
Irie-Sasaki, J.; Sasaki, T.; Matsumoto, W.; Opavsky, A.; Cheng, M.; Welstead, G.; Griffiths, E.; Krawczyk, C.; Richardson, C. D.; Aitken, K.; Iscove, N.; Koretzky, G.; Johnson, P.; Liu, P.; Rothstein, D. M.; Penninger, J. M. CD45 is a JAK phosphatase and negatively regulates cytokine receptor signalling. Nature 409: 349-354, 2001.
Inoue, I.; Taniuchi, I.; Kitamura, D.; Jenkins, N. A.; Gilbert, D. J.; Copeland, N. G.; Watanabe, T. Characteristics of the mouse genomic histamine H1 receptor gene. Genomics 36: 178-181, 1996.
Kadowaki, N.; Ho, S.; Antonenko, S.; de Waal Malefyt, R.; Kastelein, R. A.; Bazan, F.; Liu, Y.-J. Subsets of human dendritic cell precursors express different Toll-like receptors and respond to different microbial antigens. J. Exp. Med. 194: 863-869, 2001.
Kishihara, K.; Penninger, J.; Wallace, V. A.; Kundig, T. M.; Kawai, K.; Wakeham, A.; Timms, E.; Pfeffer, K.; Ohashi, P. S.; Thomas, M. L. Normal lymphocyte development but impaired cell maturation in CD45-exon6 protein tyrosine phosphatasedeficient mice. Cell 74: 143-156, 1993.
Kozlow, E. J.; Wilson, G. L.; Fox, C. H.; Kehrl, J. H. Subtractive cDNA cloning of a novel member of the Ig gene superfamily expressed at high levels in activated B lymphocytes. Blood 81: 454-461, 1993.
Ku, C. C.; Murakami, M.; Sakamoto, A.; Kappler, J.; Marrack, P. Control of homeostasis of CD8+ memory T cells by opposing cytokines. Science 288: 675-678, 2000.
Kung, C.; Pingel, J. T.; Heikinheimo, M.; Klemola, T.; Varkila, K.; Yoo, L. I.; Vuopala, K.; Poyhonen, M.; Uhari, M.; Rogers, M.; Speck, S. H.; Chatila, T.; Thomas, M. L. Mutations in the tyrosine phosphatase CD45 gene in a child with severe combined immunodeficiency disease. Nature Med. 6: 343-345, 2000.
Latour, S.; Gish, G.; Helgason, C. D.; Humphries, R. K.; Pawson, T.; Veillette, A. Regulation of SLAM-mediated signal transduction by SAP, the X-linked lymphoproliferative gene product. Nature Immun. 2: 681-690, 2001.
Le Coniat, M.; Traiffort, E.; Ruat, M.; Arrang, J.-M.; Berger, R. Chromosomal localization of the human histamine H1-receptor gene. Hum. Genet. 94: 186-188, 1994.
Lee, J.; Chuang, T.-H.; Redecke, V.; She, L.; Pitha, P. M.; Carson, D. A.; Raz, E.; Cottam, H. B. Molecular basis for the immunostimulatory activity of guanine nucleoside analogs: activation of Toll-like receptor 7. Proc. Nat. Acad. Sci. 100: 6646-6651, 2003.
Lowenthal, J. W.; Zubler, R. H.; Nabholz, M.; MacDonald, H. R. Similarities between interleukin-2 receptor number and affinity on activated B and T lymphocytes. Nature 315: 669-672, 1985.
Olavesen, M. G.; Bentley, E.; Mason, R. V.; Stephens, R. J.; Ragoussis, J. Fine mapping of 39 ESTs on human chromosome 6p23-p25. Genomics 46: 303-306, 1997.
Punnonen, J.; Cocks, B. G.; Carballido, J. M.; Bennett, B.; Peterson, D.; Aversa, G.; de Vries, J. E. Soluble and membrane-bound forms of signaling lymphocytic activation molecule (SLAM) induce proliferation and Ig synthesis by activated human B lymphocytes. J. Exp. Med. 185: 993-1004, 1997.
Reeves, R. H.; Patch, D.; Sharpe, A. H.; Borriello, F.; Freeman, G. J.; Edelhoff, S.; Disteche, C. The costimulatory genes Cd80 and Cd86 are linked on mouse chromosome 16 and human chromosome 3. Mammalian Genome 8: 581-582, 1997.
Sarkissian, M.; Winne, A.; Lafyatis, R. The mammalian homolog of suppressor-ofwhite-apricot regulates alternative mRNA splicing of CD45 exon 4 and fibronectin IIICS. J. Biol. Chem. 271: 31106-31114, 1996.
Seigel, L. J.; Harper, M. E.; Wong-Staal, F.; Gallo, R. C.; Nash, W. G.; O'Brien, S. J. Gene for T-cell growth factor: location on human chromosome 4q and feline chromosome B1. Science 223: 175-178, 1984.
Shah, R.; Banks, K.; Patel, A.; Dogra, S.; Terrell, R.; Powers, P. A.; Fenton, C.; Dinauer, C. A.; Tuttle, R. M.; Francis, G. L. Intense expression of the B7-2 antigen presentation coactivator is an unfavorable prognostic indicator for differentiated thyroid carcinoma of children and adolescents. J. Clin. Endocr. Metab. 87: 4391-4397, 2002.
Smith, K. A. Interleukin-2: inception, impact, and implications. Science 240: 1169-1176, 1988.
Taniguchi, T.; Matsui, H.; Fujita, T.; Takaoka, C.; Kashima, N.; Yoshimoto, R.; Hamuro, J. Structure and expression of a cloned cDNA for human interleukin-2. Nature 302: 305-310, 1983.
Tatsuo, H.; Ono, N.; Tanaka, K.; Yanagi, Y. SLAM (CDw150) is a cellular receptor for measles virus. Nature 406: 893-897, 2000.
Webb, G. C.; Campbell, H. D.; Lee, J. S.; Young, I. G. Mapping the gene for murine T-cell growth factor, II-2, to bands B-C on chromosome 3 and for the alpha chain of the IL2-receptor, II-2ra, to bands A2-A3 on chromosome 2. Cytogenet. Cell Genet. 54: 164-168, 1990.
Weinberg, K.; Parkman, R. Severe combined immunodeficiency due to a specific defect in the production of interleukin-2. New Eng. J. Med. 322: 1718-1723, 1990.
Yamashita, M.; Fukui, H.; Sugama, K.; Horio, Y.; Ito, S.; Mizuguchi, H.; Wada, H. Expression cloning of a cDNA encoding the bovine histamine H1 receptor. Proc. Nat. Acad. Sci. 88: 11515-11519, 1991.
Yang, J.; Patil, R. V.; Yu, H.; Gordon, M.; Wax, M. B. T cell subsets and slL-2R/IL-2 levels in patients with glaucoma. Am. J. Ophthal. 131: 421-426, 2001.
Zhou, L. J.; Schwarting, R.; Smith, H. M.; Tedder, T. F. A novel cell-surface molecule expressed by human interdigitating reticulum cells, Langerhans cells, and activated lymphocytes is a new member of the Ig superfamily. J. Immun. 149: 735-742, 1992.
Zhou, L.-J.; Tedder, T. F. Human blood dendritic cells selectively express CD83, a member of the immunoglobulin superfamily. J. Immun. 154: 3821-3835, 1995.

### SUMMARY OF INVENTION

A number of SNPs has been associated with induction of different immune responses. Some of the identified polymorphisms have been suggested in patent literature as useful in diagnosis of different immune system related disseases (see for example WO2002232928 related to polymorphisms in HRH1 gene, US2002090680 related to an allelic variant of IL-2, or WO2003045318 related to a mutation in the CD83 gene). The authors of the present invention for the first time describe herein
1) an association of the polymorphism of the SFRS8, SLAMF1, CD86, TLR7, TLR8 and TLR10 genes with a predisposition to an immune related disease;
2) an association of specific haplotypes of the identified polymorphisms with a predisposition to a particular immune related disease;
3) polymorphisms of the genes of the adjacent chromosome areas, which are in linkage disequilibrium with the identified polymorphisms, as diagnostic markers of a predisposition to an immune related desiase;
4) novel polymorphisms of the CD83, IL2 and HRH1 genes associated with a predisposition to an immune related disease;
5) a method of determining a predisposition to an immune related disease comprising determining a polymorphism of the SFRS8, SLAMF1, CD83, CD86, IL2, HRH1, TLR7, TLR8 and/or TLR10 gene;
6) a method of treating an individual having a predisposition to an immune related disease comprising inhibiting expression of a gene selected from the SFRS8, SLAMF1, CD83, CD86, IL2, HRH1, TLR7, TLR8 and/or TLR10 gene, said gene comprising a polymorphism described herein.

Accordingly, in the first aspect the invention relates to a method for determining a predisposition to an immune-related disease in a subject comprising determining in a biological sample isolated from said subject one or more polymorphisms in the chromosome regions containing the CD83 and/or SLAMF1, and/or CD86, and/or HRH1, and/or IL2, and/or TLR7, and/or TLR8, and/or TLR10 genes, or in a translational or transcriptional product from said regions, said polymorphism being indicative of said predisposition.

The inventors of the present application have discovered that polymorphisms, such as SNPs, identified in the coding and/or non-coding regions of the SFRS8 and/or CD83 and/or SLAMF1, and/or CD86, and/or IL2, and/or HRH1, and/or TLR7, and/or TLR8, and/or TLR10 genes are strongly associated to the presence or absence of a range of immune-related diseases including type 1 allergy, asthma, atopic dermatitis and rhinitis. Thus, detecting the presence or absence of the SNPs of the present invention amounts to determining a predisposition for having or not having an immune-related disease. It thus follows that determining the presence of the wild-type allele amounts to determining a predisposition for having/not having an immune-related disease. The strength of the association between the presence/absence of at least two polymorphisms in the above genes and the diseases is very strong.

Diagnosis of individuals for genetic predisposition to immuno-related diseases is important so that they can be given the best treatment and adapt their lifestyle according to their genetic predisposition.

The authors of the present invention performed haplotype analysis of the identified SNPs and found out that the coincidence of some haplotypes in association with a particular disease is higher then the coincidence of another haplotype and the disease. Thus, the invention also relates to specific haplotypes of the identified SNPs. Moreover, it is expected that with the information made available by the inventors, more polymorphisms in the SFRS8, CD83, SLAMF1, CD86, HRH1, IL2, TLR7, TLR8, and TLR10 genes will be found predisposing to immune related diseases. Therefore, all polymorphisms being in linkage disequilibrium with the identified in the application SNPs in the chromosome regions as defined in the present application are included in the scope of the protection as diagnostic markers of the predisposition for an immune-related disease, in particular an allergic disease.

In a further aspect the invention relates to isolated oligonucleotide sequences comprising at least 10 contiguous nucleotides being 100% identical to a subsequence of the SFRS8, CD83, SLAMF1, CD86, IL2, HRH1, TLR7, TLR8, and/or TLR10 genes comprising or adjacent to a polymorphism of the invention, said polymorphism or mutation being associated to an immune-related disease.

As the present inventors have determined that the SFRS8, CD83, SLAMF1, CD86, HRH1, IL2, TLR7, TLR8, and TLR10 genes are etiological factors in immune-related diseases it is important to be able to detect and correct or suppress any polymorphism in the genes which is correlated to these diseases. The isolated oligonucleotides may be used as probes for detection of the polymorphisms and/or as primer pairs for amplification of a target nucleotide sequence and/or as part of a gene therapy vector for administration to a patient suffering from immune-related diseases.

In a further aspect the invention relates to a kit for predicting an increased risk of a subject of developing immune related diseases or for other diagnostic and classification purposes of immune related diseases comprising at least one probe comprising at least two nucleic acid sequences as defined above.

These kits which may further comprise buffers and primers and reagents can be used for diagnosing the polymorphisms and mutations which correlate to immune-related diseases.

The invention also relates to SFRS8, SLAMF1, CD86, IL2, HRH1, TLR7, TLR8, and TLR10 variant proteins comprising mutations which correspond to the identified in the application polymorphisms of the corresponding genes. These variant proteins may also be used for diagnosis of immune-related diseases.

According to a further aspect the invention relates to antibodies capable of selectively binding to the variant proteins as defined above with a different (such as lower or higher) binding affinity than when binding to the polypeptide having the amino acid sequence of wild type protein.

These antibodies may be used in diagnosing individuals with the polymorphisms. It is also envisaged that such specific antibodies may be used for treating patients carrying the mutated protein.

In further aspects the present invention relates to methods of treating patients suffering from immune related disorders, in particular allergic disorders. Among the therapeutic methods, one method relates to a method of treating immune related diseases in a subject being diagnosed as having a predisposition according to the invention, comprising administering to said subject a therapeutically effective amount of a gene therapy vector. The invention also relates to a gene therapy vector itself, said vector being capable of altering the polymorphism in cells of a subject being diagnosed as having a predisposition according to the invention, or being capable of correcting, suppressing, supporting or changing the expression of the SFRS8, CD83, SLAMF1, CD86, IL2, HRH1, TLR7, TLR8, and/or TLR10 genes in cells of a subject suffering from said diseases.

With the advent of gene therapy it has become possible to suppress and/or to eliminate the effects of a polymorphism by administering to a subject a gene therapy vector which either alters the polymorphism or suppresses the transcription and/or translation from the gene. Such gene therapy vectors have the advantage of being highly specific.

The present invention also relates to
a compound capable of inhibiting expression of a gene selected from the SFRS8, CD83, SLAMF1, CD86, IL2, HRH1, TLR7, TLR8, and/or TLR10 genes, wherein said gene comprises a SNP indicative of a predisposition to an immune related disease, and/or capable of inhibiting the activity of a product of said gene.
use of a compound as above for the manufacture of a medicament for treatment of an immune related disease selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema.
a pharmaceutical composition for the treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, comprising a compound of above.
a method of treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, comprising administering a compound or a pharmaceutical composition as above.

Further, the invention relates
to a method of screening for a candidate compound for therapeutic treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said method comprising an in vitro or in vivo model system comprising an immune related gene wherein the gene is comprising a polymorphism associated with said immune related disease,
to a method for prognosis of the likelihood of development of an immune related disease comprising determining a polymorphism associated with predisposition to said immune related disease,
to a method of predicting the likelihood of a subject to respond to a therapeutic treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said method comprising determining the genotype of said subject in the chromosome areas comprising the SFRS8, CD83, SLAMF1, CD86, HRH1, IL2, TLR7, TLR8 and/or TLR10 gene.

### FIGURE LEGENDS

FIG. 1 Statistical analysis of the association of different haplotypes of the SNPs identified in the SLAMF1 gene and SNPs of the CD84 and CD48 gene being in linkage disequilibrium with the SNPs of the SLAMF1 gene with predisposition to asthma in two independent samples of 100 and 143 Danish sibpair families (AIA and VB correspondingly) showing p-values obtained by the transmission disequilibrium test (TDT). Alleles of the individual SNPs are indicated for every phenotype; indicates under expression of a haplotype, “+”-over expression of a haplotype.

FIG. 2 Statistical analysis of the association of different haplotypes of the SNPs identified in the SLAMF1 gene and SNPs of the CD84 and CD48 gene being in linkage disequilibrium with the SNPs of the SLAMF1 gene with predisposition to asthma accompanied with increased specific IgE (RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 3. Statistical analysis of the association of different haplotypes of the SNPs identified in the SLAMF1 gene and SNPs of the CD84 and CD48 gene being in linkage disequilibrium with the SNPs of the SLAMF1 gene with predisposition to increased specific IgE (RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 4. Statistical analysis of the association of different haplotypes of the SNPs identified in the SLAMF1 gene and SNPs of the CD84 and CD48 gene being in linkage disequilibrium with the SNPs of the SLAMF1 gene with predisposition to atopic dermatitis and/or atopic dermatitis (AD) accompanied with the increased specific IgE (RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 5 Statistical analysis of the association of different haplotypes of the SNPs identified in the SLAMF1 gene and SNPs of the CD84 and CD48 gene being in linkage disequilibrium with the SNPs of the SLAMF1 gene with predisposition to rhinitis (RH) and/or rhinitis accompanied with the increased specific IgE (RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 6 Statistical analysis of the association of different haplotypes of the SNPs identified in the SLAMF1 gene and SNPs of the CD84 and CD48 gene being in linkage disequilibrium with the SNPs of the SLAMF1 gene with predisposition to positive skin test (skin) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 7 Statistical analysis of the association of different haplotypes of the SNPs identified in the SLAMF1 gene and SNPs of the CD84 and CD48 gene being in linkage disequilibrium with the SNPs of the SLAMF1 gene with predisposition to increased specific IgE (RAST) and/or Type 1 allergy (Type 1) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 8 Statistical analysis of the association of different haplotypes of the SNPs identified in the HRH1 gene with predisposition to asthma (Asthma) and/or asthma accompanied with the increased specific IgE (Asthma+RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 9 Statistical analysis of the association of different haplotypes of the SNPs identified in the HRH1 gene with predisposition to increased specific IgE (RAST) and/or positive skin test (skin) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 10 Statistical analysis of the association of different haplotypes of the SNPs identified in the HRH1 gene with predisposition to atopic dermatitis (AD) and/or atopic dermatitis accompanied with the increased specific IgE (AD+RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 11. Statistical analysis of the association of different haplotypes of the SNPs identified in the HRH1 gene with predisposition to rhinitis (RH) and/or rhinitis accompanied with the increased specific IgE (RH+RAST) in two independent samples of 100 and 143 Danish sibpair families (VB and AIA correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 12 Statistical analysis of the association of different haplotypes of the SNPs identified in the TLR7 gene and SNPs of the TLR8 gene with predisposition to asthma in two independent samples of 100 and 143 Danish sibpair families (AIA and VB correspondingly) showing p-values obtained by the transmission disequilibrium test (TDT). Alleles of the individual SNPs are indicated for every phenotype; indicates under expression of a haplotype, “+”—over expression of a haplotype.

FIG. 13 Statistical analysis of the association of different haplotypes of the SNPs identified in the TLR7 gene and SNPs of the TLR8 gene with predisposition asthma accompanied with the increased specific IgE (Asthma+RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 14 Statistical analysis of the association of different haplotypes of the SNPs identified in the TLR7 gene and SNPs of the TLR8 gene with predisposition to increased specific IgE (RAST) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 15 Statistical analysis of the association of different haplotypes of the SNPs identified in the TLR7 gene and SNPs of the TLR8 gene with predisposition to increased specific IgE (RAST), Type 1 allergy (Type 1) and/or positive skin teast (skin) in two independent samples of 100 and 143 Danish sibpair families (AIA and VB correspondingly) showing p-values obtained by the transmission disequilibrium test (TDT). Alleles of the individual SNPs are indicated for every phenotype; indicates under expression of a haplotype, “+”—over expression of a haplotype.

FIG. 16 Statistical analysis of the association of different haplotypes of the SNPs identified in the TLR7 gene and SNPs of the TLR8 gene with predisposition to rhinitis (RH) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 17 Statistical analysis of the association of different haplotypes of the SNPs identified in the TLR7 gene and SNPs of the TLR8 gene with predisposition to rhinitis (RH) accompanied with the increased specific IgE (RH+rast) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 18 Statistical analysis of the association of different haplotypes of the SNPs identified in the TLR7 gene and SNPs of the TLR8 gene with predisposition to atopic dermatitis (AD) and/or atopic dermatitis accompanied with the increased specific IgE (AD+rast) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”-over expression of a haplotype.

FIG. 19 Statistical analysis of the association of different haplotypes of the SNPs identified in the TLR10 gene with predisposition to asthma and/or asthma accompanied with the increased specific IgE (Asthma+rast) in two independent samples of 100 and 143 Danish sibpair families (AIA and VB correspondingly) showing p-values obtained by the transmission disequilibrium test (TDT). Alleles of the individual SNPs are indicated for every phenotype; “−” indicates under expression of a haplotype, “+”—over expression of a haplotype.

FIG. 20 Statistical analysis of the association of different haplotypes of the SNPs identified in the TLR10 gene with predisposition to atopic dermatitis (AD) and/or atopic dermatitis accompanied with the increased specific IgE (AD+rast) in two independent samples of 100 and 143 Danish sibpair families (AIA and VB correspondingly) showing p-values obtained by the transmission disequilibrium test (TDT). Alleles of the individual SNPs are indicated for every phenotype.; “−” indicates under expression of a haplotype, “+”—over expression of a haplotype.

FIG. 21 Statistical analysis of the association of different haplotypes of the SNPs identified in the TLR10 gene with predisposition to increased specific IgE (RAST), rinitis (RH), rhinitic accompanied with the increased specific IgE (ARH+rast), positive skin test (skin), and/or type 1 allergy (Type 1) in two independent samples of 100 and 143 Danish sibpair families (AIA and VB correspondingly) showing p-values obtained by the transmission disequilibrium test (TDT). Alleles of the individual SNPs are indicated for every phenotype.

FIG. 22. Statistical analysis of the association between CD86 ile179val and allergy phenotypes, showing p-values obtained by the transmission disequilibrium test (TDT) Sample 1 and 2 represent two independent samples of 100 and 143 Danish sibpair families, respectively. (abbreviations: AD—atopic dermatitis; rast—increased specific IgE (RAST≧1+); Ast—asthma; Rh—Rhinitis; NS—not significant)

### DEFINITIONS

### Gene/Gene Sequence

A compilation of:
the genomic sequences which are transcribed into a transcriptional entity
the genomic sequences in between
the genomic sequences involved in regulation of expression and splicing of the gene comprising at least 2000 bp upstream and downstream from the transcribed entity.

“Immune related gene” is in the present context a gene which expression is associated with normal and/or pathologic activity of the immune system, in particular is associated with proliferation, maturation and/or activation of T and/or B lymphocytes.

The present invention relates to the genes identified in the NCBI database (http://www.ncbi.nlm.nih.gov) as

### GeneID: 6504 (SLAMF1)

### GeneID: 942 (CD86)

### GeneID: 9308 (CD83)

### GeneID: 3269 (HRH1)

### GeneID: 3358 (IL2)

### GeneID: 51284 (TLR7)

### GeneID: 51311 (TLR8)

### GeneID: 81793 (TLR10)

### GeneID: 51284 (TLR7)

### GeneID: 6433 (SFRS8)

Genomic sequences of the above genes (http://genome.ucsc.edu/) are identified in the present invention as

| | |
|---|---|
| SLAMF1 gene | SEQ ID NO: 1 |
| CD86 gene | SEQ ID NO: 2 |
| CD83 gene | SEQ ID NO: 3 |
| HRH1 gene | SEQ ID NO: 4 |
| IL2 gene | SEQ ID NO: 5 |
| TLR7 gene | SEQ ID NO: 6 |
| TLR8 gene | SEQ ID NO: 7 |
| TLR10 gene | SEQ ID NO: 8 |
| SFRS8 gene | SEQ ID NO: 9 |

The term “chromosome region containing a gene” means a part of a human chromosome containing a gene of the invention and the nucleotide sequences adjacent to both ends of the gene, i.e. SEQ ID NO: 1-8 or 9, wherein one end of the gene corresponds to the first nucleotide of the gene sequence, and another end corresponds to the last nucleotide of the gene sequence.

The term “adjacent” is used in connection with
(i) a gene sequence to indicate a nucleotide sequence/chromosome region that is located sufficiently close to said gene sequence in a chromosome, such as for instance less then 10 000, e.g. less then 9 000, such as less then 8 000, e.g. less then 7 000, such as less then 6 000, e.g. from 1 000 to 5 000, e.g. 2 000 or 1 000 nucleotide positions. It is preferred that the adjacent region is in linkage disequilibrium with said gene sequence;
(ii) a oligonucleotide sequence to indicate that the oligonucleotide recognises a sequence that is sufficiently closely located to a specific nucleotide of interest for the oligonucleotide to be suitable for the desired detection technique, such as for instance as a primer for amplification of a target nucleotide sequence. Preferably, adjacent means less than 500, such as less than 400, e.g. less than 300, such as less than 200, e.g. less than 100, such as less than 50 nucleotide positions away from the nucleotide or nucleotide sequence of interest.

As used herein, the term “coding sequence” refers to that portion of a gene that encodes an amino acid sequence of a protein. Exons constitute the coding sequence of the gene.

Coding sequences of the above genes are identified in the present invention as SEQ ID NO: 10 (SLAMF1), SEQ ID NO: 11 (CD86), SEQ ID NO: 12 (CD83), SEQ ID NO: 13 (HRH1), SEQ ID NO: 14 (IL2), SEQ ID NO:15 (TLR7), SEQ ID NO: 16 (TLR8), SEQ ID NO: 17 (TLR10), SEQ ID NO: 18 (SFRS8).

The promoter and intron regions referred herein as the “non-coding region(s)/sequence(s)” of the given genes. As used herein, “intron” refers to a DNA sequence present in a given gene that is spliced out during mRNA maturation. The term “promoter region” refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase.

The term “fragment” when used in connection with nucleotide sequences means any fragment of the nucleotide sequence consisting of at least 20 consecutive nucleotides of that sequence.

As used herein, the term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. Such polymorphism is referred herein as “single nucleotide polymorphism” or SNP. A polymorphic region also can be several nucleotides in length. The present invention relates to polymorphisms which may be an insertion, deletion and/or substitution of one or more additional nucleotides in the sequence of a gene. A gene having at least one polymorphic region is referred as “polymorphic gene”.

SNPs, which are known in the art, are identified herein with the numbers corresponding to the refSNP ID NOs (rs numbers) of the NCBI SNP database (http://www.ncbi.nlm.nih.gov/SNP/) and UCSC Genome SNP database (http://www.genome.ucsc.edu/), for example such as rs3796504, rs2295619, rs12076998, rs1000807, rs2295613, rs1171285, rs346074, rs901865, rs2069763, rs2069762, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233, rs1379049.

SNPs, which are not described in the art and do not have refSNP ID NOs in the NCBI database, are identified herein with the names indicating their location in the gene structure, for example “ex 3a”, “prom 2” or “ex 3c”, wherein “ex” or “prom” means the exon or promoter correspondingly, “3a”, “2” or “3c” indicates a particular exon or promoter of the gene. It is to be understood that the SNPs identified hereinwith the latter names are described herein for the first time,

As used herein, “allele”, which is used interchangeably herein with “allelic variant” refers to alternative forms of a gene or portions thereof. Alleles occupy the same locus or position on homologous chromosomes. When an individual has two identical alleles of a gene, the individual is said to be homozygous for the gene or allele. When an individual has two different alleles of a gene, the individual is said to be heterozygous for the gene or alleles. Alleles of a specific gene can differ from each other in a single nucleotide, or several nucleotides, and can include substitutions, deletions, and insertions of nucleotides. An allele of a gene also can be a form of a gene containing a mutation.

As used herein, “predisposition” means that an individual having a particular geno-type and/or haplotype has a higher likelihood than one not having such a genotype and/or haplotype for a particular condition/disease as one of the described herein.

As used herein, the term “haplotype” refers to a set of closely linked genetic markers present on one chromosome which tend to be inherited together (not easily separable by recombination). Some haplotypes may be in linkage disequilibrium.

As used herein, the term “genetic marker” refers to an identifiable physical location on a chromosome (e.g., single nucleotide polymorphism (SNP), restriction enzyme cutting site) whose inheritance can be monitored. Markers can be expressed regions of DNA (genes) or some segment of DNA with no known coding function but whose pattern of inheritance can be determined.

As used herein, the term “linkage” refers to an association in inheritance between genetic markers such that the parental genetic marker combinations appear among the progeny more often than the non-parental.

As used herein, the term “linkage disequilibrium” (LD) means that the observed frequencies of haplotypes in a population does not agree with haplotype frequencies predicted by multiplying the frequencies of individual genetic markers in each haplotype; LD means that there exist correlations among neighbouring alleles, reflecting ‘haplotypes’ descended from single, ancestral chromosomes.

### Allergic Diseases/Disorders:

Asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

### Immune-Related Diseases:

All of the above allergic diseases and infectious diseases, autoimmune diseases, graft/host incompatibilities.

ASTHMA (MIM 600807) comprises a syndrome of bronchial inflammation, hyperesponsiveness and airflow obstruction. The use of the term allergic asthma as the basic term for asthma mediated by immunologic mechanisms seems relevant and may outdate the classic classification of intrinsic versus extrinsic asthma.

BRONCHIAL HYPERRESPONSIVENESS (BHR) is by convention demonstrated if an individual's FEV decreases by 20% form the baseline after inhaled histamine or metacholine in standard concentrations. In some studies BHR is used to strengthen the asthma diagnosis since it is included in the asthma definition utilised by the American Thoracic Society.

RHINITIS (MIM 607154) or hay fever is defined as an inflamation of the lining of the nose and is characterized by nasal itching and blockage, rhinorrhea and sneezing. Rhinoconjunctivitis also includes conjunctival itching and increased tear fluid in addition to symptoms of rhinitis. Symptoms are in some definitions considered abnormal if lasting for at least one hour a day on most days.

ATOPIC DERMATITIS (MIM 603165) is a chronic relapsing dermatitis associated with high levels of IgE and often co-existing with specific allergies. It is diagnosed according to the Hanifin-Rajka criteria or later established diagnostic criteria.

ATOPY is a commonly used phenotype in the investigation of allergy genetics. Generally atopy is regarded as a disorder of IgE response to common environmental allergens, associated with clinical allergic disease, and detectable by measurement of either total serum IgE, specific IgE or skin prick test. A recent attempt to reserve the word atopy to describe a clinical trait and predisposition proposed the definition: Atopy is a personal or familial tendency to produce IgE antibodies in response to low doses of allergens, usually proteins, and to develop typical symptoms such as asthma, rhinoconjunctivitis, or eczema/dermatitis.

The TOTAL SERUM IGE level is associated with allergy and can be analysed as a quantitative or semi-quantitative trait and solely or in combination with other phenotypes. Usually a total serum IgE level of 100 kU/I is considered to be increased.

Target nucleic acid: a nucleic acid isolated from an individual and comprising at least one polymorphism identified in the present invention as well as further nucleotides upstream or downstream. The target nucleic acid can be used for hybridisation, for sequencing or other analytical purposes.

Alignment. When reference is made to alignment of protein sequences alignment is carried out using the MultAlin algorithm with default settings (“Multiple sequence alignment with hierarchical clustering”, F Corpet, 1988, Nucl. Acids Res., 16 (22), 10881-10890), which is available at the internet address: http:/prodes.toulouse.inra.fr/multalin/multalin.html.

### Amino Acid Substitutions:

Substitutions within the below identified groups of amino acids are considered as conservative amino acid substitutions; substitutions of amino acids between the different groups are considered as non-conservative amino acid substitutions:

P, A, G, S, T (neutral, weakly hydrophobic)
Q, N, E, D, B, Z (hydrophilic, acid amine)
H, K, R (hydrophilic, basic)
F, Y, W (hydrophobic, aromatic)
L, I, V, M (hydrophobic)
C (cross-link forming)

### DETAILED DESCRIPTION OF THE INVENTION

### 1. Gene Polymorphism

The first aspect of the invention relates to a method for determining a predisposition to an immune-related disease or condition in a subject comprising determining in a biological sample isolated from said subject two or more polymorphisms in the chromosome regions containing an immune related gene such as the SFRS8 and/or SLAMF1, and/or CD86, and/or CD83, and/or HRH1, and/or IL2, and/or TLR7, and/or TLR8, and/or TLR10 genes, or in a translational or transcriptional product from said regions, said polymorphism being indicative of said predisposition.

### 1.1 Position of Polymorphisms

In one embodiment the present invention relates to two or more polymorphisms in the above identified genes, wherein the polymorphisms are located in the non-coding regions of the genes, such as an intron region or a region controlling expression of the genes, e.g. a promotor region. Such polymorphisms according to the invention may influence expression of the gene or affect the splicing or maturation of the gene transcript, mRNA.

In another embodiment the invention relates to polymorphisms locates in the coding regions of the gene, such as an exon. Such polymorphisms according to invention may lead to the production of variant proteins.

Variant proteins are the proteins amino acid sequence of which contains an amino acid change, e.g. an amino acid substitution, insertion and/or deletion, which corresponds to the polymorphism of a gene. A variant protein may have an altered functional activity due to the latter polymorphism.

Thus, in one aspect the present invention relates to a method for determining a predisposition to an immune related disease comprising determining two or more polymorphisms in the chromosome regions containing the SFRS8, SLAMF1, CD86, TLR7, TLR8, TLR10, CD83, IL2 and/or HRH1 genes and relating said polymorphisms to a predisposition to an immune related disease. Two or more polymorphisms may be located either/both in a coding region and/or non-coding region of any of said genes. In one embodiment the polymorphisms may be located in one individual gene selected from the SFRS8, SLAMF1, CD86, TLR7, TLR8, TLR10, CD83, IL2 and/or HRH1 genes. In another embodiment the polymorphisms may be located in two or more different genes selected from the latter genes. According to these embodimnets at least two polymorphisms in the identified genes are to be determined.

In other embodiments of the invention, a method for determining a predisposition to an immune-related disease may comprise determining one or more polymorphisms in the above identified genes. Thus, in these embodiments the determining a predisposition to an immune-related disease may comprise determining one single polymorphism in any of the above identified genes. The polymorphism may be located i) in a coding region of the gene, ii) in a non-coding region of the gene. The examples of such polymorphhisms are discussed below.

Thus, according to the invention a predisposition to an immune-related disease may comprises determining two or more polymorphisms in any of the identified herein genes, or it may be determined by determined a single polymorphism in a gene selected form the genes identified above.

When determining at least two polymorphisms, in one embodiment the polymorphisms may be located within the nucleotide sequences of the SLAMF1 and CD86 genes. In another embodiment the polymorphisms may be located in the sequences of the SLAMF1 and HRH1 genes. In another embodiment the polymorphisms located in the nucleotide sequences of the SLAMF1 and TLR7 genes may be determined. In still another embodiment the invention relates to determining the polymorphisms located in the nucleotide sequences of the SLAMF1 and TLR8 genes. In yet another embodiment the invention relates to determining the polymorphisms located in the SLAMF1 and TLR10 genes. In still yet another embodiment the invention relates to determining the polymorphisms located in the SLAMF1 and IL2 genes. Also, the at least two polymorphisms may be determined in the SLAMF1 and CD83 genes or in the SLAMF1 and SFRS8 genes.

In other embodiments of the invention may concern determining at least two polymorphisms located in the sequences containing the genes
i) CD86 and HRH1, or
ii) CD86 and IL2, or
iii) CD86 and CD83, or
iv) CD86 and TLR7, or
v) CD86 and TLR8, or
vi) CD86 and TLR10,
vii) CD86 and SFRS8.

Still, in other embodiments, the at least two polymorphisms may be located in any two genes selected from the SFRS8, SLAMF1, CD86, CD83, HRH1, IL2, TLR7, TLR8, and TLR10 genes.

In a preferred embodiment the invention relates to polymorphisms, wherein at least one of the polymorphisms is a single nucleotide polymorphism, SNP.

The invention relates to SNPs having refSNP Nos rs3796504, rs2295619, rs12076998, rs1000807, rs2295613, rs1171285, rs346074, rs901865, rs2069763, rs2069762, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992 and rs755437.

In some embodiments a preferred SNP may be selected from the SNPs having refSNP Nos: rs3796504, rs2295612, rs12076998, rs1000807, rs2295613, rs179008, rs5743781, rs864058, rs5741883, rs3764879, rs3764880, rs5744077, rs2159377, rs2407992, rs11466657, rs11466655, rs11096955, rs11096956, rs11096957, rs11466645, rs11466642, rs1171285, rs346074 or rs901865

In other embodiments a preferred SNP may be selected from the SNPs having refSPN Nos. rs3796504, rs2295612, rs12076998, rs1000807, rs2295613, rs179008, rs5743781, rs864058, rs5741883, rs3764879, rs3764880, rs5744077, rs2159377, rs2407992, rs11466657, rs11466655, rs11096955, rs11096956, rs11096957, rs11466645 and rs11466642.

In still some other embodiments a preferred SNP may be selected from the SNOs having refSPN Nos. rs755437, rs1051219, rs1051233, rs1379049 or rs378288.

A preferred SNP may also be an SNP identified herein as

ex 1b (of the SLAMF1 gene),
prom 2 (of the CD83 gene),
ex 5 (of the CD 86 gene) or
ex 3a (of the TLR10 gene).

The latter SNPs are particular preferred when a method for determining a predisposition for an immune related disease comprises determining at least one polymorphism in the SFRS8, SLAMF1, CD86, or TLR10 genes or in the chromosome regions containing the SFRS8, SLAMF1, CD86, or TLR10 genes.

Thus, a particular SNP or a group of SNPs may be selected when a particular immune related gene of the invention is concerned. For example
rs3795504, rs2295612, rs12076998, rs1000807 and/or rs2295613 may be determined when a method for determining a predisposition to an immune related disease is concerned the determining a polymorphism in the chromosome regions containing the SLAMF1 gene and/or and/or in the chromosome regions containing the SLAMF1 and relating said polymorphism to the predisposition;
rs2067470, rs901865, rs346074 and/or rs1171285 may be determined when a method for determining a predisposition to an immune related disease is concerned the determining a polymorphism the HRH1 gene and/or in the chromosome regions containing the HRH1 and relating said polymorphism to the predisposition;
rs864058, rs5743781 and/or rs179008 may be preferred when a method for determining a predisposition to an immune related disease is concerned the determining a polymorphism in the chromosome regions containing the TLR7 gene and relating said polymorphism to the predisposition;
rs2407992, rs2159377, rs5744077, rs3764880, rs3764879 and/or rs5741883 may be determined when a method for determining a predisposition to an immune related disease is concerned the determining a polymorphism in the TLR8 gene or in the chromosome regions containing the TLR8 gene and relating said polymorphism to the predisposition;
rs11466642, rs11466645, rs1109696, rs11096955, rs11466655, and/or rs11466657 may be determined when a method for determining a predisposition to an immune related disease is concerned the determining a polymorphism in the TLR10 gene and/or in the chromosome regions containing the TLR10 gene and relating said polymorphism to the predisposition,
rs755437, rs378288, rs1051219, rs1051233 and/or rs1379049 of the SFRS8 gene may be determined when a method for determining a predisposition to an immune related disease is concerned the determining a polymorphism in the SFRS8 gene and/or in the chromosome regions containing the SFRS8 gene and relating said one polymorphism to the predisposition;
rs2069763 or rs2069762 of the IL2 gene may be determined when a method for determining a predisposition to an immune related disease is concerned the determining a polymorphism in the IL2 gene and/or in the chromosome regions containing the IL2 gene and relating said one polymorphism to the predisposition.

Positions of the above identified SNPs within the genomic sequences of the genes (SEQ ID NOS: 1-9) are identified in Table 1 below:

| | | | Nucleotide No | |
|---|---|---|---|---|
| | SEQ | | (position | |
| Gene | ID NO | SNP ID | of SNP) | SNP |
| SLAMF1 | 1 | rs3796504 | 157797341 | C/A (reverse strand) |
| SLAMF1 | 1 | rs2295612 | 157833495 | C/A (reverse strand) |
| SLAMF1 | 1 | ex 1b | 157833534 | G/T |
| SLAMF1 | 1 | rs12076998 | 157833560 | T/C (reverse strand) |
| SLAMF1 | 1 | rs1000807 | 157833820 | G/T |
| SLAMF1 | 1 | rs2295613 | 157833923 | C/T |
| CD86 | 2 | ex 5 | 52986 | A/G |
| CD83 | 3 | prom 2 | 14225259 | C/T |
| HRH1 | 4 | rs1171285 | 11269027 | C/A (reverse strand) |
| HRH1 | 4 | rs346074 | 11269310 | G/A (reverse strand) |
| HRH1 | 4 | rs901865 | 11275707 | G/A (reverse strand) |
| IL2 | 5 | rs2069763 | 123836303 | A/C |
| IL2 | 5 | rs2069762 | 123836801 | G/T |
| TLR7 | 6 | rs179008 | 12265085 | A/T |
| TLR7 | 6 | rs5743781 | 12266396 | G/A (reverse strand) |
| TLR7 | 6 | rs864058 | 12267456 | A/G |
| TLR8 | 7 | rs5741883 | 12285647 | G/A (reverse strand) |
| TLR8 | 7 | rs3764879 | 12286123 | C/G |
| TLR8 | 7 | rs3764880 | 12286252 | A/G |
| TLR8 | 7 | rs5744077 | 12298613 | T/C (reverse strand) |
| TLR8 | 7 | rs2159377 | 12298939 | C/T |
| TLR8 | 7 | rs2407992 | 12300538 | C/G |
| SFRS8 | 9 | rs755437 | 130926532 | C/T |
| SFRS8 | 9 | rs1051219 | 130732199 | C/T |
| SFRS8 | 9 | rs1051233 | 130745161 | G/C |
| SFRS8 | 9 | rs1379049 | 130701038 | G/A |
| SFRS8 | 9 | rs3782288 | 130872819 | A/G |
| TLR10 | 8 | rs11466657 | 38672974 | C/T |
| TLR10 | 8 | rs11466655 | 38673250 | A/G |
| TLR10 | 8 | rs11096955 | 38673287 | T/G (reverse strand) |
| TLR10 | 8 | rs11096956 | 38673360 | T/G (reverse strand) |
| TLR10 | 8 | rs11096957 | 38673671 | C/A (reverse strand) |
| TLR10 | 8 | ex 3a | 38674558 | A/C |
| TLR10 | 8 | rs11466645 | 38675383 | A/T |
| TLR10 | 8 | rs11466642 | 38675435 | A/G |

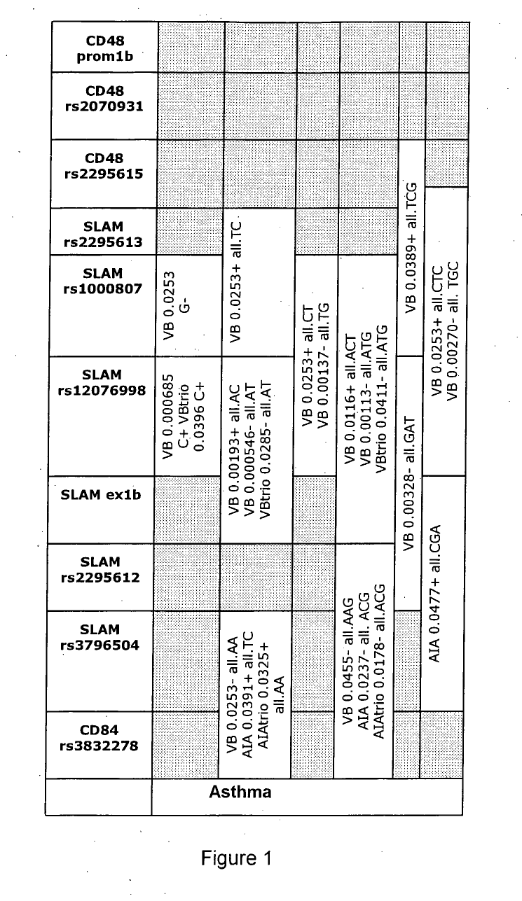

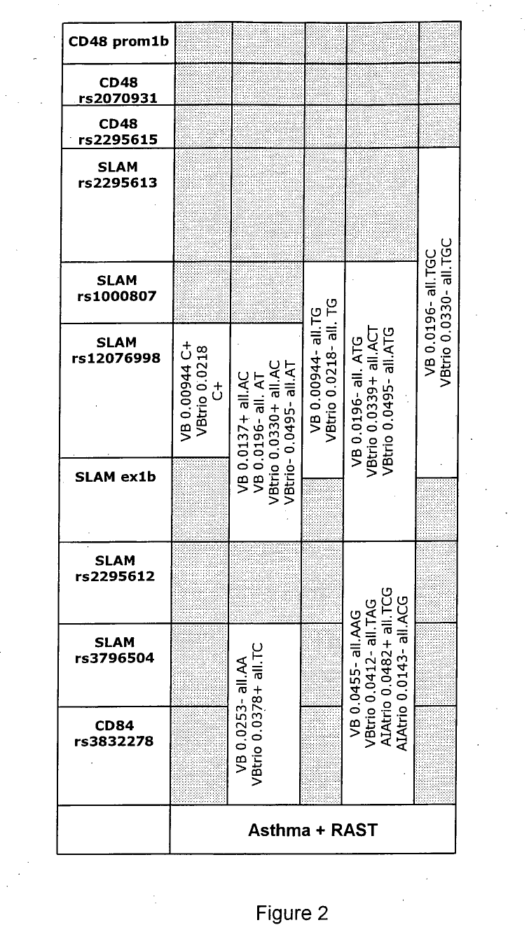

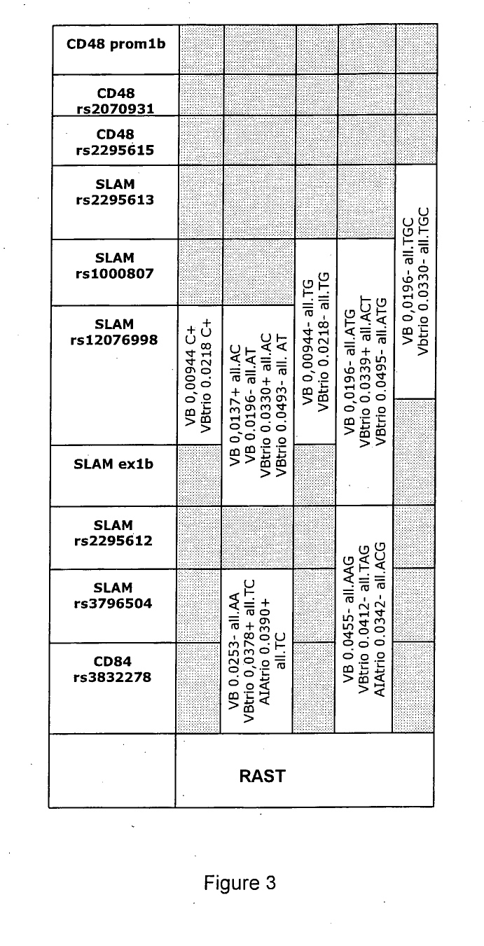

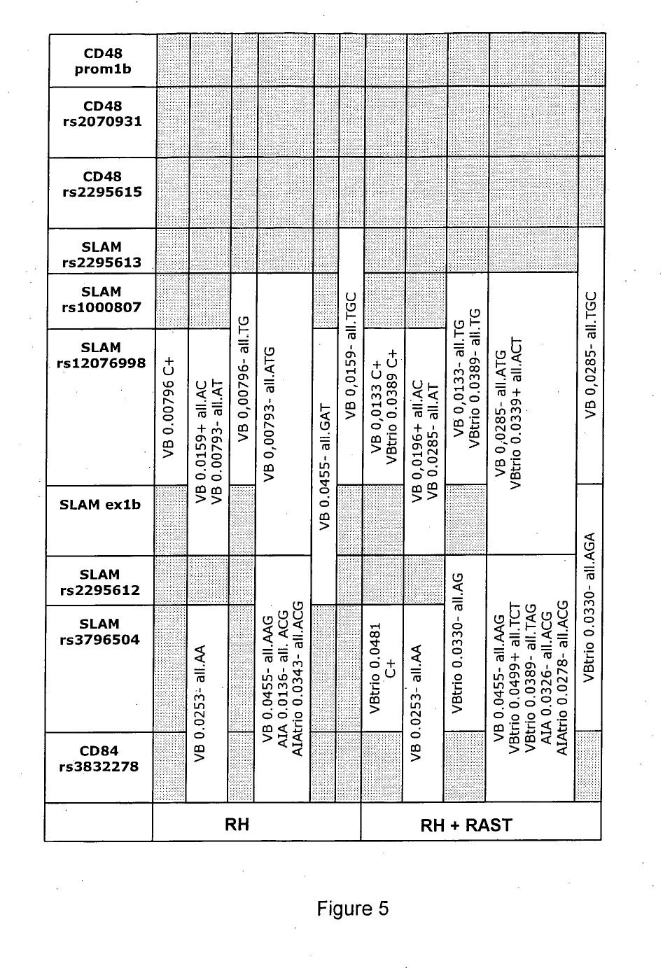

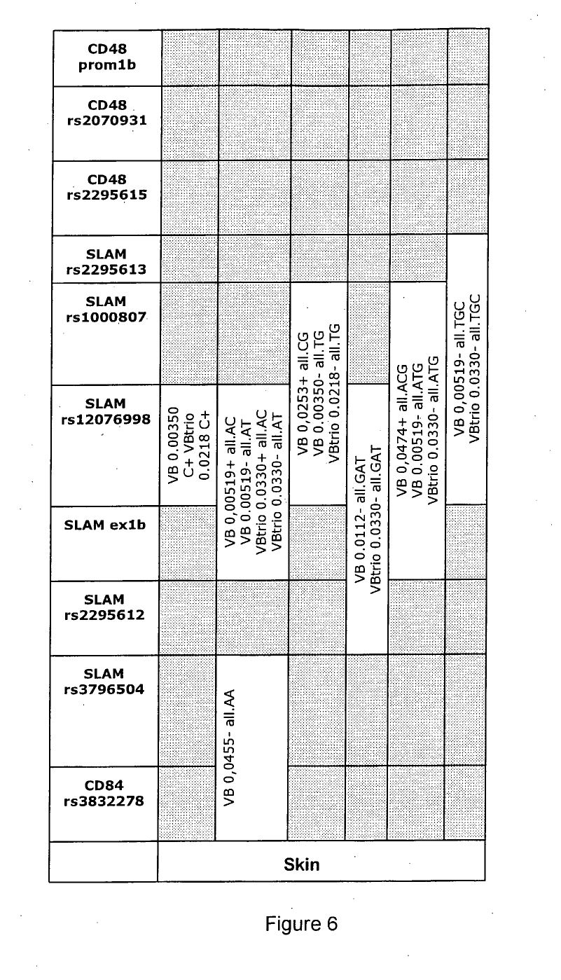

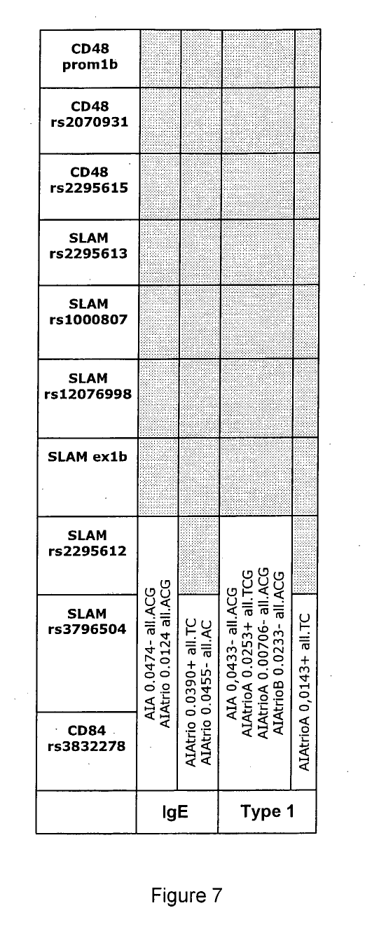

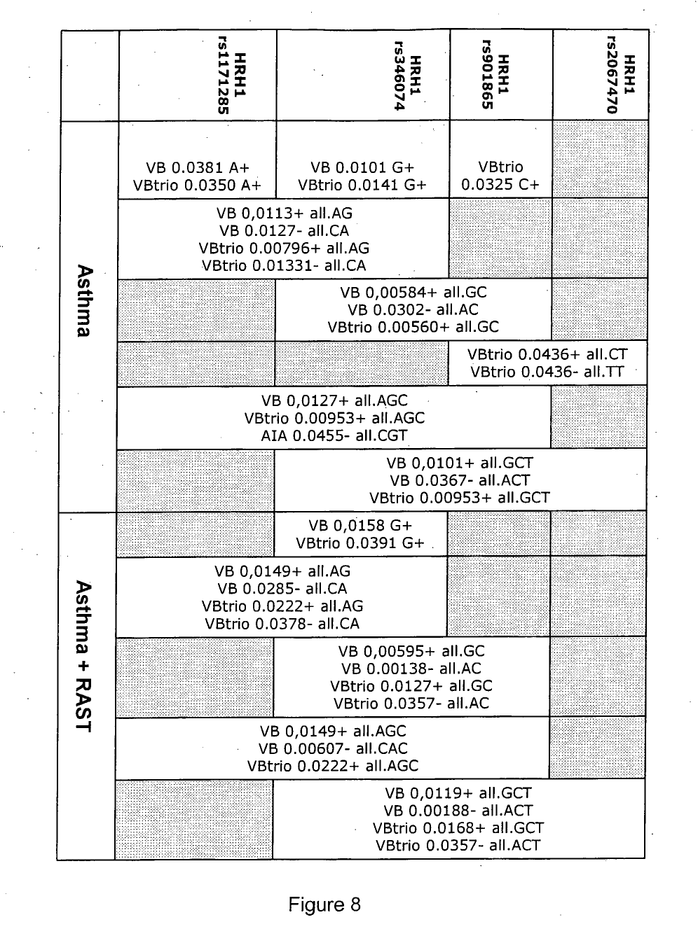

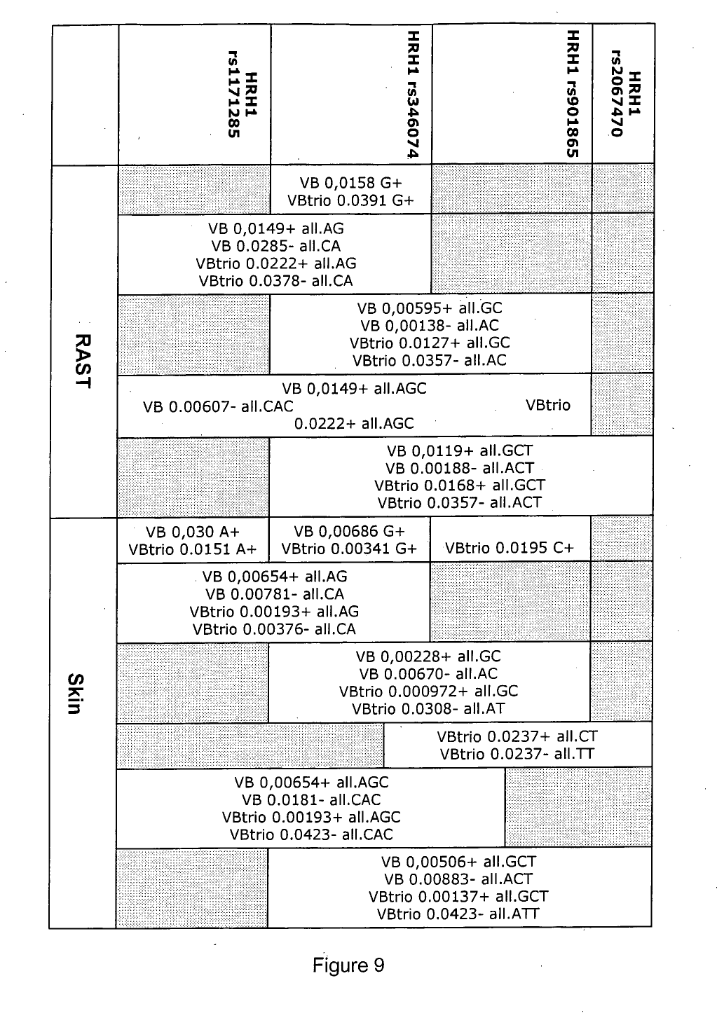

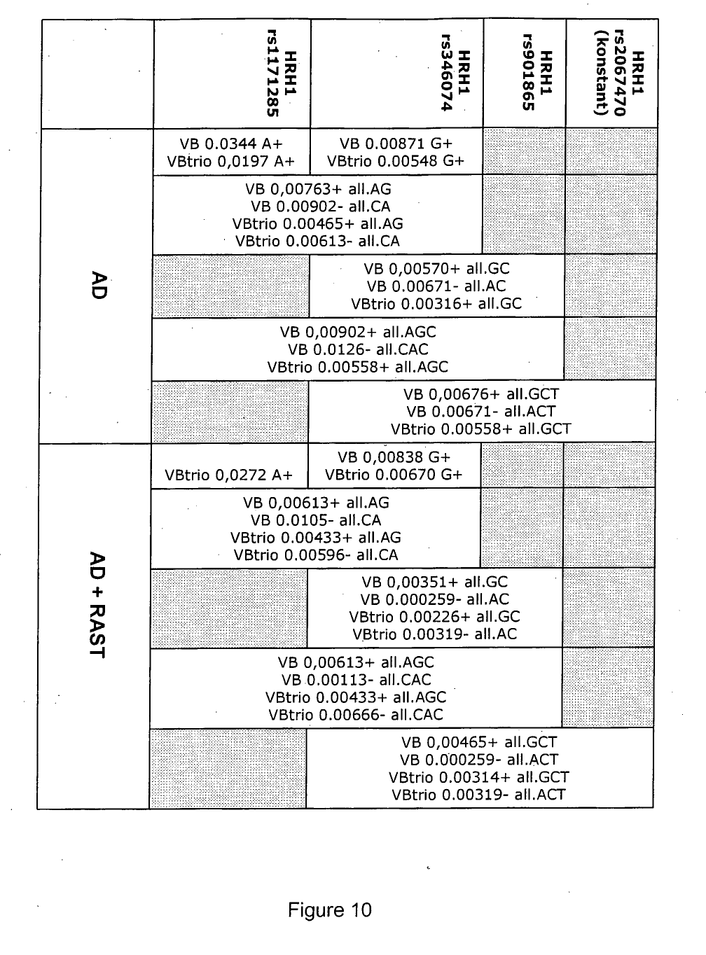

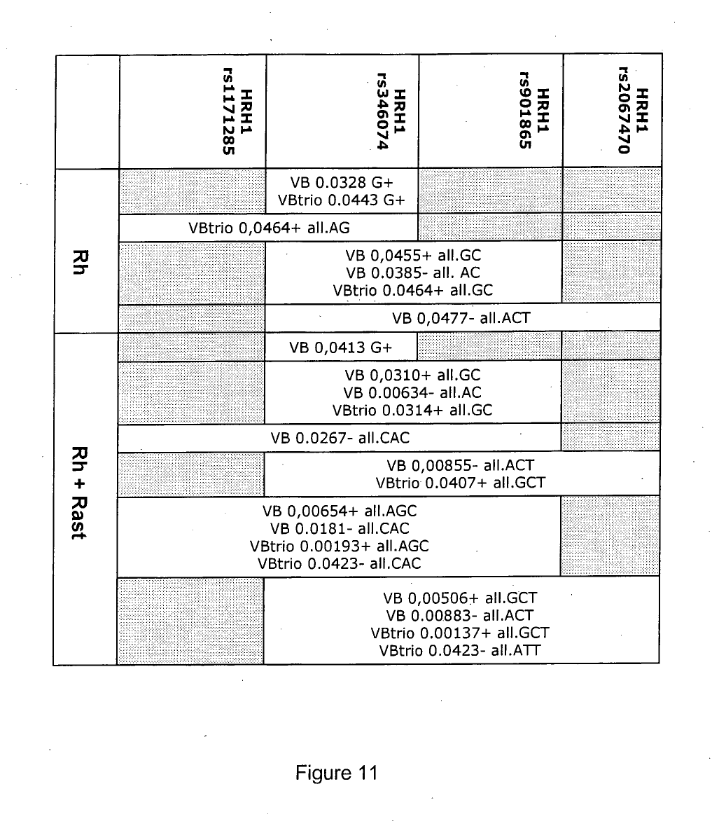

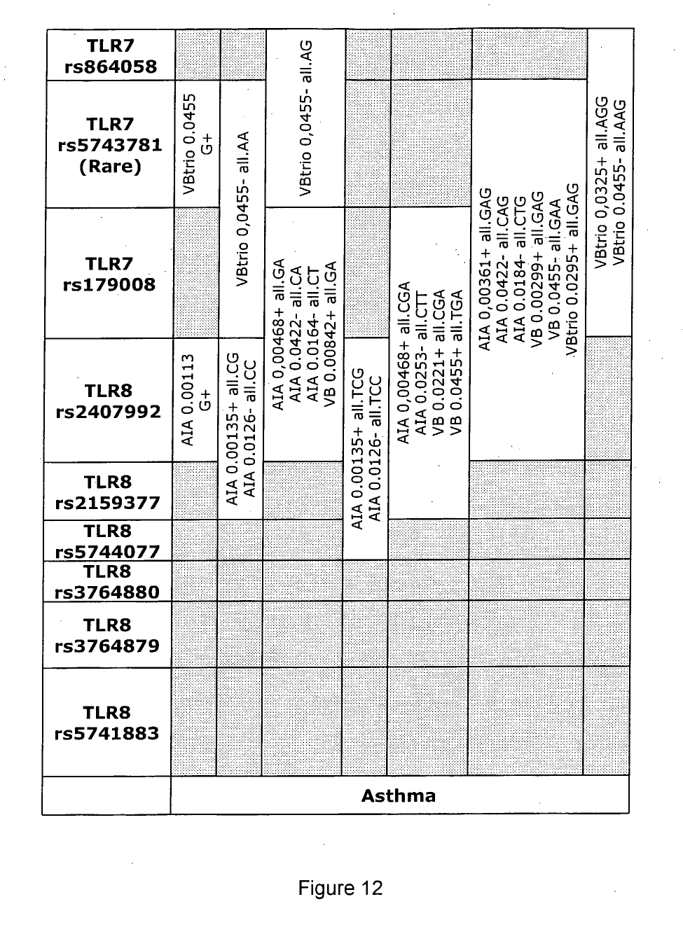

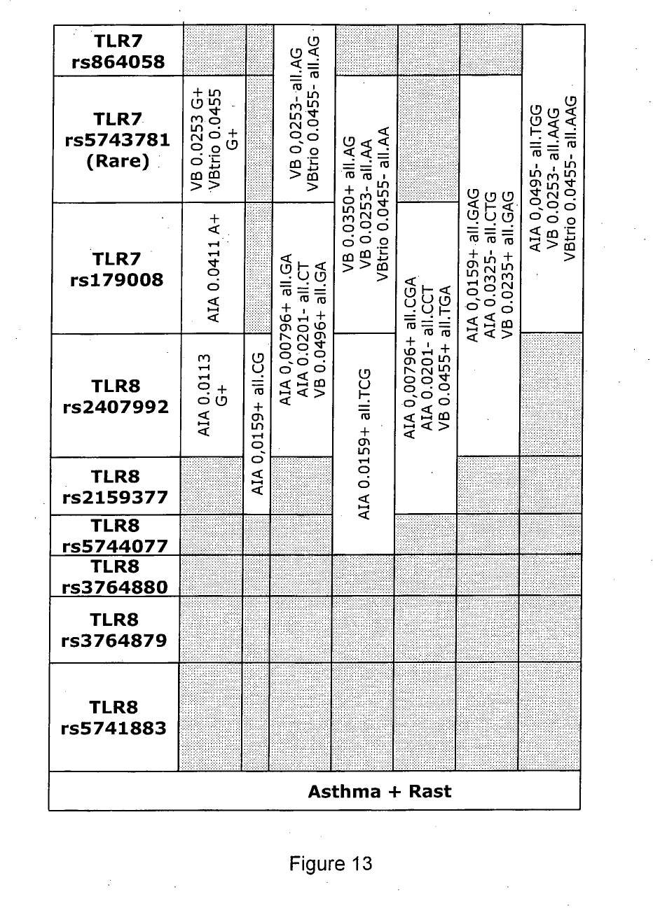

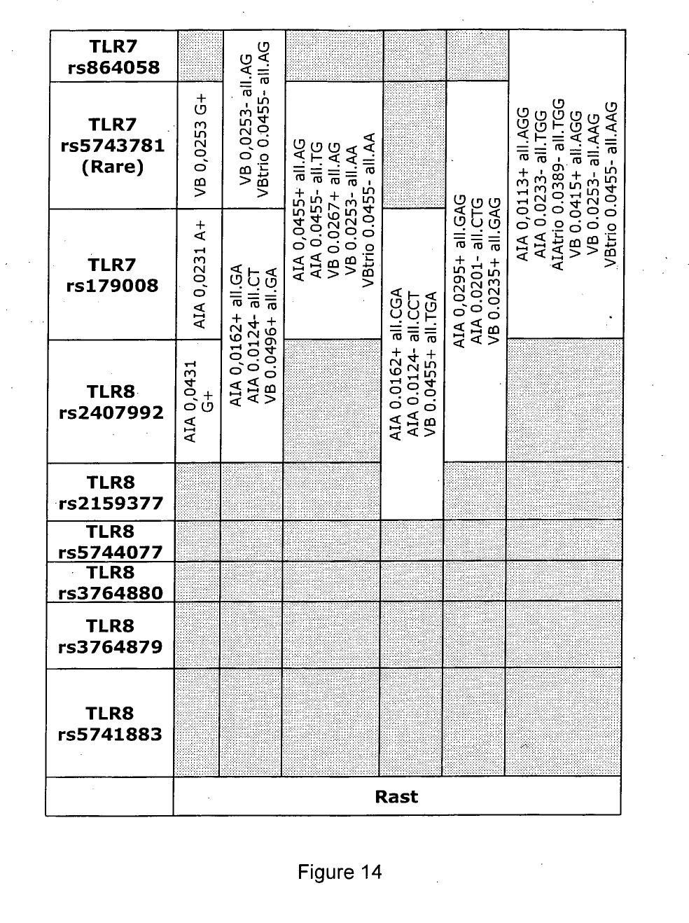

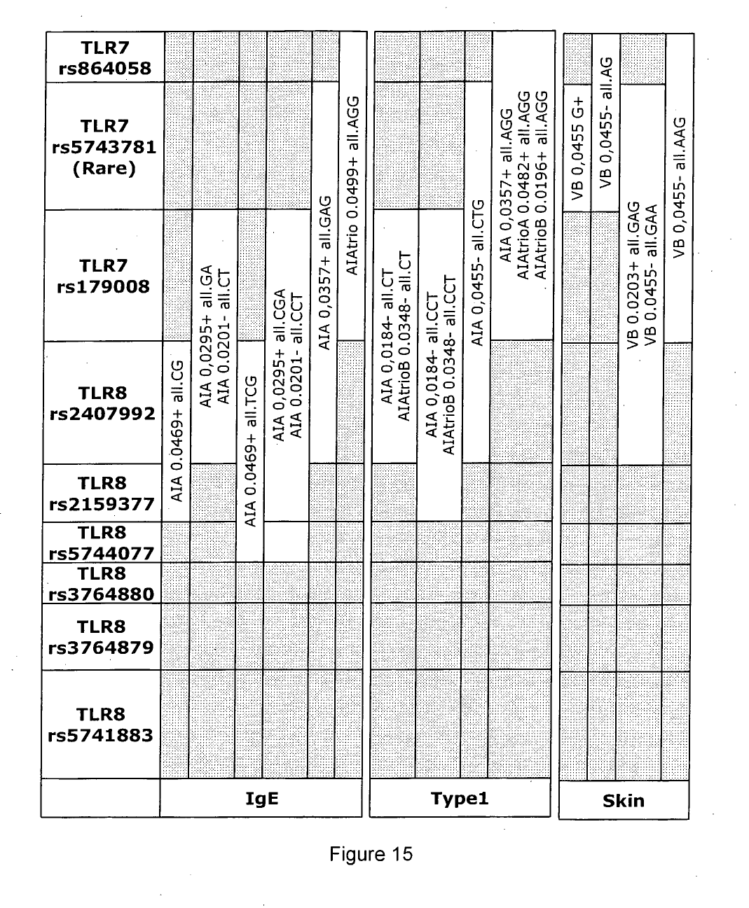

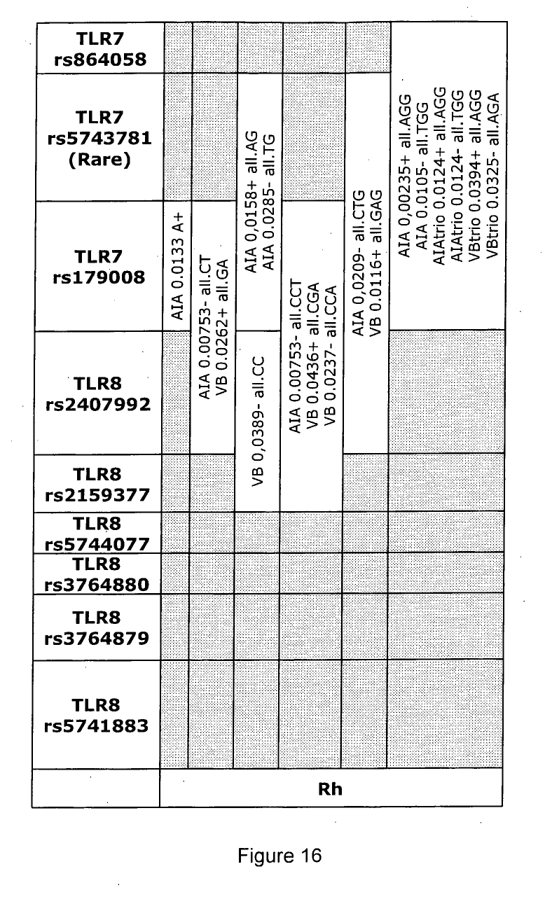

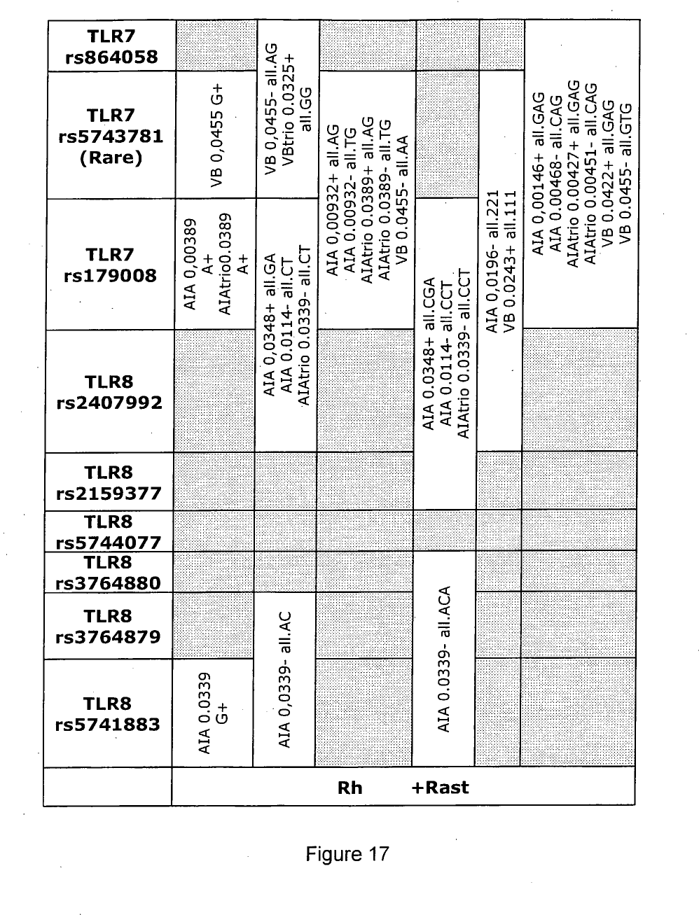

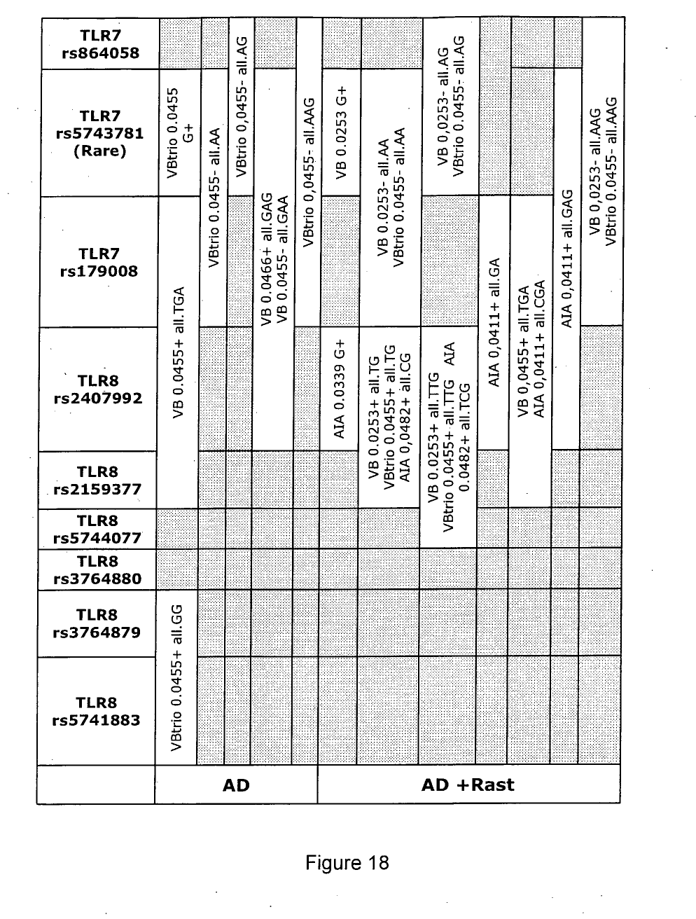

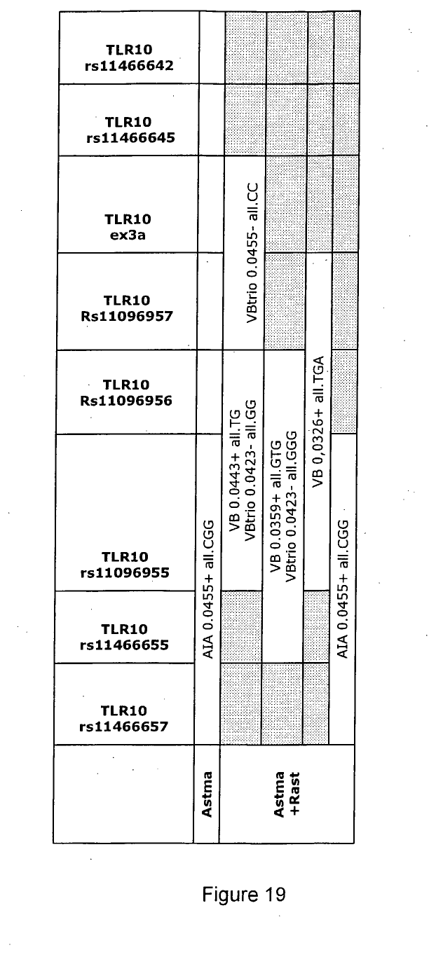

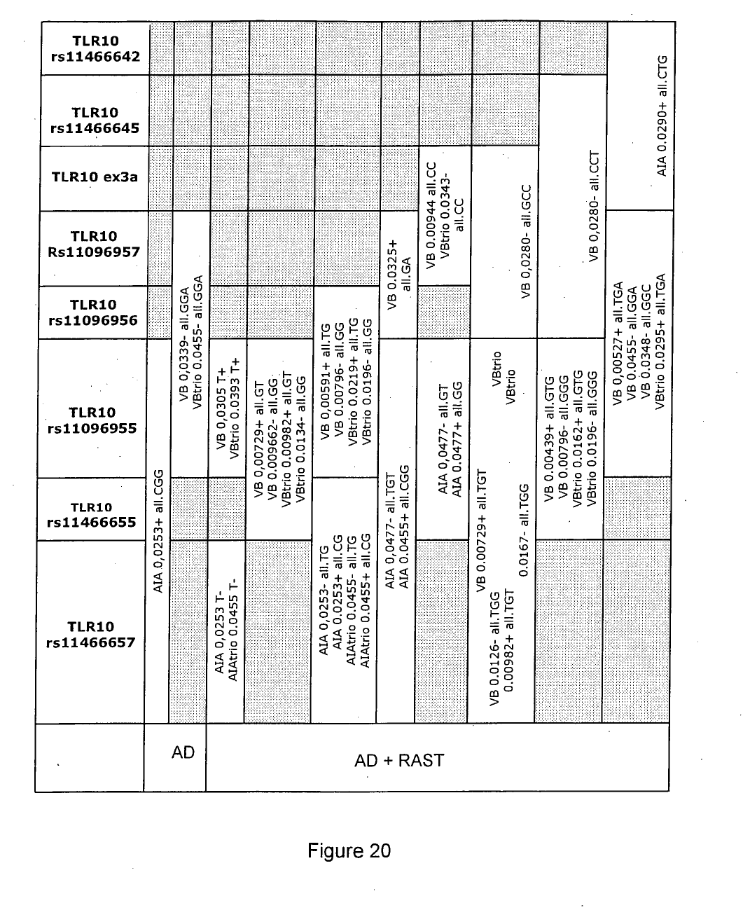

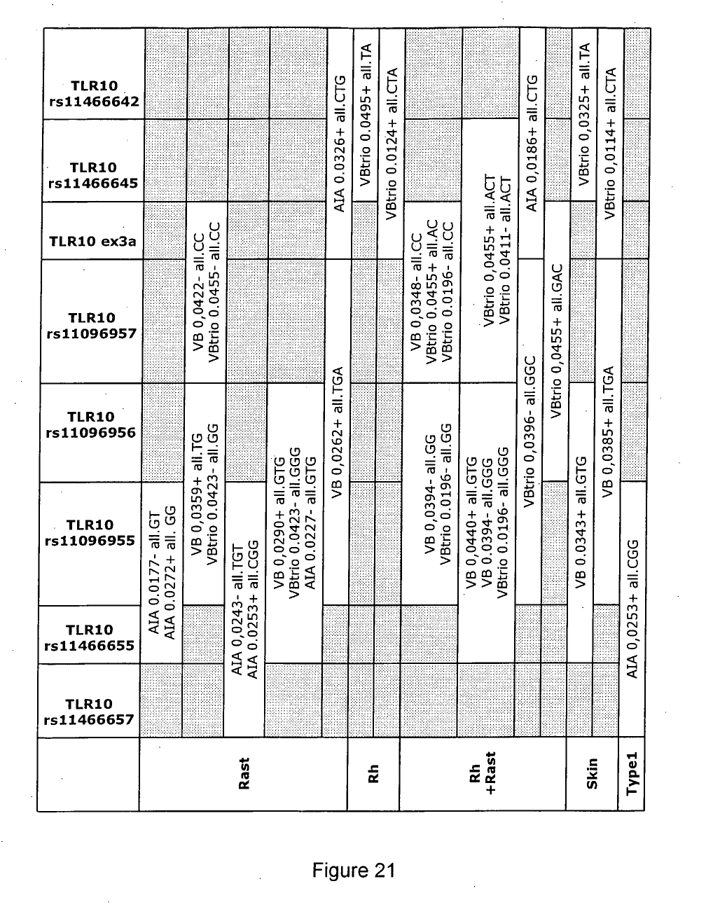

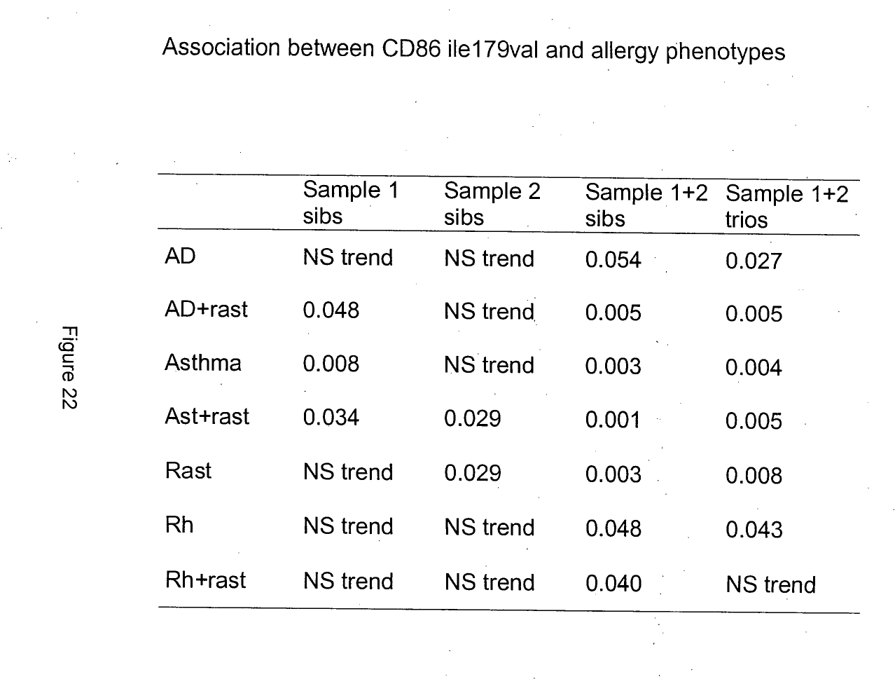

According to the invention the above SNPs are genetic markers of immune-related diseases of the invention described below. The invention also features haplotypes of the above SNPs the presence of which is strongly correlated with a particular immune related disease. Thus, the invention also relates to haplotypes which are in linkage disequilibrium. Examples of particular haplotypes of the invention which are associated with particular immune-related diseases are presented in FIGS. 1-22 of the present application and Table 5 below.

In another aspect the invention relates to polymorphisms located in the chromosome regions containing the above identified genes, wherein said polymorphisms are in linkage disequilibrium with at least one of the above identified SNPs. Thus, the invention relates to any polymorphisms in the regions of human chromosomes 1q22-q23, 3q21, 4p14, 12q24, 6p23, 3p21-p14, Xp22.3, Xp22, containing a gene of the invention which are in linkage disequilibrium with any of the SNPs identified above, for example, such as polymorphisms in the human chromosome 3q which are in linkage disequilibrium with the CD86 gene, such as polymorphisms in the CD80 gene. The present inventors have determined a signal from the region containing the CD80 gene. This gene is located approximately 2.5 Mb from the CD86 gene and it is possible that this signal is linked to the polymorphism detected in the CD86 gene. It may also be that the signal from CD80 contributes independently to the physiological condition of the subjects. However, any polymorphism in a region of the human chromosome 3q adjacent to the CD86 gene which is in linkage disequilibrium with the CD86 gene and correlated to a predisposition for a disease or a protection against immune-related diseases is included in the scope of the invention.

The invention includes in the scope any polymorphism in any SFRS8, SLAMF1, CD83, CD86, TLR7, TLR8, TLR10, IL2 or HRH1 neighbouring gene located within approximately 2.5 Mb upstream or downstream to said genes, said neighbouring gene being in linkage disequilibrium with any of the genes of the invention. For example, the invention relates to polymorphisms in the regions of the human chromosome 1q which are in linkage disequilibrium with the SLAMF1 gene, such as polymorphisms in the CD48 and CD84 genes. The CD48 and CD84 are the SLAMF1 neighbouring genes. The invention preferably relates to single nucleotide polymorphisms in the latter genes. More particular the invention relates to SNPs having refSNP Nos. rs3832278, rs2295615, rs2070931 and rs 2295613. However, the invention relates to any polymorphism of the human chromosome 1q within approximately 2.5 Mb upstream or dowmstream of the SLAMF1 gene in case this polymorphism is in linkage disequilibrium with the SLAMF1 gene and if the polymorphism correlates with a predisposition to a immune related disease or a protection against an immune related disease described in the present application.

Any polymorphism of the genes being adjacent to the genes of the invention, such as polymorphisms located within the distantce of 500 to 10 000 nucleotides to/from an immune reletaed gene of the invention and is in linkage disequilibrium with the SNPs identified above, is in the scope of the invention.

A polymorphism being a SNP located within the sequence of 2000-2500 nucleotides juxtaposed to the first and/or to the last nucleotide of a genomic sequence identified herein as SEQ ID NOs: 1-9 are preferred. However, polymorphism of non-immune or other immune related genes, which interact with any of the genes of the invention, such as presented in the following table are also included in the scope of the invention as indicative of the presence of a predisposition to an immune related disease of the invention:

| | | Allele | | |
|---|---|---|---|---|
| Gene | Variation | Protective | Risk | Reference |
| GSTM1 | deletion of large | having | having | Brasch-Andersen C et |
| | part of gene | two | zero | al. Hum Mutat. 2004 |
| | | copies | copies | September; 24(3): 208-14. |
| GSTT1 | deletion of large | having | having | Brasch-Andersen C_et |
| | part of gene | two | zero | al. Hum Mutat. 2004 |
| | | copies | copies | September; 24(3): 208-14. |
| PHF11 | haplotype | | | Zhang Y et al., Nat |
| | | | | Genet. 2003(2): 181-6. |
| DPP10 | haplotype | | | Allen M et al., Nat |
| | | | | Genet. 2003(3): 258- |
| | | | | 63. |
| HLA-G | SNP C1489T | | | Nicolae, D. et al. Am. |
| | haplotype | | | J. Hum. Genet. 76: |
| | | | | 349-357, 2005 |
| | | | | Nicolae, D. et al. Am. |
| | | | | J. Hum. Genet. 76: |
| | | | | 349-357, 2005 |
| ADAM33 | Haplotype | | | Van Eerdewegh, P et |
| | | | | al. Nature 418: 426- |
| | | | | 430, 2002 |
| Interleukin-2B | SNP 4237G-A | A | G | Randolph et al Am. J. |
| | | | | Hum. Genet. 75: 709- |
| | | | | 715, 2004 |
| Interleukin-9 | sDF2*10 | | sDF2*10 | Kauppi, P et al., Eur. |
| receptor | | | | J. Hum. Genet. 8: 788- |
| | | | | 792, 2000 |
| KCNS3 | SNP rs1031771 | | G | Hao K et al. Hum |
| | SNP rs1031772 | | T | Genet. 2005 |
| | | | | April; 116(5): 378-83 |
| Interleukin-4 | −589C/T | | | Sandford A J et al., J |
| | | | | Allergy Clin Immunol |
| | | | | 2000; 106: 135-40 |
| Interleukin-4R | SNP S503P¹ | Q | R | Howard, T. D et al., |
| | SNP Q576R | | Ile | Am. J. Hum. Genet. |
| | Ile50Val | | | 70: 230-236, 2002 |
| | | | | Khurana Hershey, G K |
| | | | | et al., New Eng. J. |
| | | | | Med. 337: 1720-1725, |
| | | | | 1997 & Deichmann, |
| | | | | K A et al., Clin. Exp. |
| | | | | Allergy 28: 151-155, |
| | | | | 1998 |
| | | | | Mitsuyasu, H. et al., |
| | | | | Nature Genet. 19: |
| | | | | 119-120, 1998 |
| Interleukin-13 | SNP A4464G | A | G | Heinzmann, H et al., |
| | SNP Arg130Gln | Arg | Gln | Hum. Molec. Genet. 9: |
| | SNP −1111C/T | C | T | 549-559, 2000 |
| | | | | Vladich, F et al., J. |
| | | | | Clin. Invest. 115: 747- |
| | | | | 754, 2005 & Wang, M |
| | | | | et al., Hum. Genet. |
| | | | | 113: 387-390, 2003. |
| | | | | Howard, T D et al., Am. |
| | | | | J. Resp. Cell Molec. |
| | | | | Biol. 25: 377-384, |
| | | | | 2001 |
| Tumor necrosis | SNP −308G/A | | A | Witte, J S et al., Eur. J. |
| factor | | | | Hum. Genet. 10: 82- |
| | | | | 85, 2002 |
| STAT6 | GT repeat in | 16-GT | 13-GT | Gao, P. S et al., J. |
| | exon1 | | | Med. Genet. 41: 535- |
| | | | | 539, 2004 |
| GRPA | SNP522363 | | C | Laitinen t et al., |
| | | | | Science. 2004 |
| | | | | 304(5668): 300-4. |
| FcεRI-β | SNP I181L | I | L | Shirakawa T et al., Nat |
| | SNP E237G | E | G | Genet 7(2): 125-9, |
| | SNP −109C/T | C | T | 1994 |
| | | | | Hill M R & Cooksom |
| | | | | WOCM Hum Mol |
| | | | | Genet 5: 959-62, 1996 |
| | | | | Hizawa N et al., Am J |
| | | | | Repir Crit Care Med |
| | | | | 161: 906-9, 2000 |
| β2Adrenoreceptor | Gly16Arg | | Gly | Ramsay C E et al., |
| | | | | Hum Genet 1999; |
| | | | | 104: 269-274 |
| STAT6 | SNP G2964A | | A | Gao P S et al., J Med |
| | | | | Genet 2000; 37(5): 380-2 |

¹A significant gene-gene interaction between S503P in IL4RA and the −1111 promoter variation in IL13 was also been detected. Individuals with the risk genotype for both genes were at almost 5 times greater risk for the development of asthma compared to individuals with both nonrisk genotypes. Howard, T. D et al., Am. J. Hum. Genet 70: 230-236, 2002

By the term “interacting gene” is meant a gene which activity or activity of a product of which is dependent on the activity of a gene of the invention; or a gene which activity or activity of a product of which is synergistic or antagonistic with activity of a gene of the invention. The invention relates to an immune related gene activity, such as for example activity associated with proliferation, differentiation and/or activation of T and/or B lymphocytes.

### 1.2 Products of the Genes

The invention relates to a method for determining a predisposition to an immune related disease comprising determining two or more polymorphisms in any of the above described genes or in transcriptional or translational products of the genes, or determining at least one of the SNPs identified herein.

As used herein, the term “transcriptional product of the gene” refers to an premessenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

As used herein, the term “translational product of the gene” refers to a protein, which is encoded by the gene.

Thus, the invention includes in the scope of protection nucleic acids comprising the coding nucleotide sequences of the above genes comprising a polymorphism and proteins comprising a polymorphism corresponding to the polymorphism of the encoding nucleic acid sequence.

In particular, the invention relates to transcriptional products of the above genes being
(i) nucleic acid sequences identified in the invention as SEQ ID NO: 10-18, or fragments thereof,
(ii) nucleic acid sequences having at least 90% identity with SEQ ID NO: 10-18, or fragments thereof,
(iii) nucleic acid sequences being complementary to any of the sequences of (i) or (ii),
   said nucleic acid sequences comprising the polymorphisms of the genomic sequences described above associated with a predisposition with an immune related disease.

Translational products of the genes of the invention are defined as
(i) variant proteins corresponding to the proteins identified under in the NCBI database under Ass. Nos.: NP_{—}003028 (SLAMF1), NP_{—}999387 (CD86), NP_{—}004224 (CD83), NP_{—}000852 (HRH1), NP_{—}000577 (IL2), NP_{—}057646 (TLR7), NP_{—}619542 (TLR8), NP_{—}112218 (TLR10), NP_{—}004583 (SFRS8) or fragments thereof, said variant proteins, fragments thereof comprising polymorphisms corresponding to the polymorphisms of the corresponding genomic sequences or transcriptional products thereof;
(ii) polypeptide sequences having at least 90% identity with the variant proteins, or fragments thereof, of (i), said polypeptide sequences comprising polymorphisms corresponding to the polymorphisms of the corresponding variant proteins.

Selected, but non-limited examples of variant proteins of the invention are given in Table 2 below:

| Gene | SNP ID. | Protein polymorphism |
|---|---|---|
| SLAMF1 | rs3796504 | Pro333Thr |
| SLAMF1 | rs2295612 | Phe11Leu |
| TLR7 | rs179008 | Gln11Leu |
| TLR7 | rs5743781 | Val448Ala |
| TLR10 | rs11466657 | Ile473Thr |
| TLR10 | rs11466655 | Gly381Asp |
| TLR10 | rs11096955 | Ile369Leu |
| TLR10 | rs11096957 | Asn241His |
| CD86 | ex 5 | Ile179Val |

A method for determining a predisposition to an immune related disease according to the invention may include the mesuating expression level of a gene of the invention, such as mesuaring expression level a transcriptional produt of the gene, or it may include mesuaring activity of another gene which is dependednt on activity of a gene of the invention. For example the expression level of the SFRS8 gene and/or the activity of the product of the SFRS8 gene may be mesuared, e.g. by monitoring the alternative splicing of the SFRS8 target gene, the CD45-gene or products thereof.

### 2. Methods of Determining Polymorphisms

### 2.1 SNP

Many methods (see Table 3 below) are known in the prior art for determining the presence of particular nucleotide sequences or for determining particular proteins having particular amino acid sequences. All of these methods may be adapted for determining the polymorphisms according to the present invention.

TABLE 3

| Method | Result |
|---|---|
| Restriction fragment length | Cleavage or non-cleavage based on |
| polymorphism | SNP results in difference in length |
| Amplified fragment length | Cleavage or non-cleavage based on |
| polymorphism | SNP results in difference in length |
| Mass spectrometry | Difference in molecular weight of hybrids |
| | between a probe and the different alleles |
| Single strand conformation | Different separation in gel based on |
| polymorphism (SSCP). SSCP | different conformation caused by single |
| heteroduplex. | nucleotide polymorphism. |
| single nucleotide extension | Difference in signal through incorporation |
| | of differently labelled nucleotide or |
| | labelled/non-labelled nucleotide |
| sequencing | Difference in sequence |
| hybridisation | Hybridisation or non-hybridisation at high |
| | stringency. Often detected by using |
| | differently labelled probes. |
| Determination of Tₘ profile | difference in Tₘ profile between target |
| | and homologous vs. non-homologous |
| | probe. |
| Cleavage of single-stranded | |
| DNA | |
| Denaturing HPLC | DHPLC is based on resolving |
| | heteroduplex from homoduplex DNA |
| | fragments produced by PCR |
| | amplification using temperature- |
| | modulated heteroduplex analysis. |
| TAQMAN | PCR based technique. |

One common method for detecting SNPs comprises the use of a probe bound to a detectable label. By carrying out hybridisation under conditions of high stringency it is ensured that the probe only hybridises to a sequence which is 100% complementary to the probe. According to the present invention this method comprises hybridising a probe to a target nucleic acid sequence comprising at least one of the SNPs at the positions identified in Table 1 (see above). For other polymorphisms or mutations within the defined region, similar probes can be designed by the skilled practitioner and used for hybridisation to a target nucleic acid sequence. The design and optimisation of probes and hybridisation conditions lies within the capabilities of the skilled practitioner.

In the scope of the present invention the term “hybridisation” signifies hybridisation under conventional hybridising conditions, preferably under stringent conditions, as described for example in Sambrook et al., Molecular Cloning, A Laboratory Manual, 2nd Edition (1989) Cold Spring Harbor Laboratory Press, Cold Spring Harbor, N.Y.). The term “stringent” when used in conjunction with hybridisation conditions is as defined in the art, i.e. 15-20° C. under the melting point Tₘ, cf. Sambrook et al, 1989, pages 11.45-11.49. Preferably, the conditions are “highly stringent”, i.e. 5-10° C. under the melting point Tₘ. Under highly stringent conditions hybridisation only occurs if the identity between the oligonucleotide sequence and the locus of interest is 100%, while no hybridisation occurs if there is just one mismatch between oligonucleotide and DNA locus. Such optimised hybridisation results are reached by adjusting the temperature and/or the ionic strength of the hybridisation buffer as described in the art. However, equally high specificity may be obtained using high-affinity DNA analogues. One such high-affinity DNA analogues has been termed “locked nucleic acid” (LNA). LNA is a novel class of bicyclic nucleic acid analogues in which the furanose ring conformation is restricted in by a methylene linker that connects the 2′-O position to the 4′-C position. Common to all of these LNA variants is an affinity toward complementary nucleic acids, which is by far the highest reported for a DNA analogue (Orum et al. (1999) Clinical Chemistry 45, 1898-1905; WO 99/14226 EXIQON). LNA probes are commercially available from Proligo LLC, Boulder, Colo., USA. Another high-affinity DNA analogue is the so-called protein nucleic acid (PNA). In PNA compounds, the sugar backbone of an oligonucleotide is replaced with an amide containing backbone, in particular an aminoethylglycine backbone. The nucleobases are retained and are bound directly or indirectly to aza nitrogen atoms of the amide portion of the backbone (Science (1991) 254: 1497-1500).

Various different labels can be coupled to the probe. Among these fluorescent reporter groups are preferred because they result in a high signal/noise ratio.

Suitable examples of the fluorescent group include fluorescein, Cy2, Cy3, Cy3.5, Cy5, Cy5.5, Cy7, acridin, Hoechst 33258, Rhodamine, Rhodamine Green, Tetramethylrhodamine, Texas Red, Cascade Blue, Oregon Green, Alexa Fluor, europium and samarium.

Another type of labels are enzyme tags. After hybridisation to the target nucleic acid sequence a substrate for the enzyme is added and the formation of a coloured product is measured. Examples of enzyme tags include a beta-Galactosidase, a peroxidase, horseradish peroxidase, a urease, a glycosidase, alkaline phosphatase, chloramphenicol acetyltransferase and a luciferase.

A further group of labels include chemiluminescent group, such as hydrazides such as luminol and oxalate esters.

A still further possibility is to use a radioisotope and detect the hybrid using scintillation counting. The radioisotope may be selected from the group consisting of ³²P, ³³P, ³⁵S, ¹²⁵I, ⁴⁵Ca, ¹⁴C and ³H.

One particularly preferred embodiment of the probe based detection comprises the use of a capture probe for capturing a target nucleic acid sequence. The capture probe is bound to a solid surface such as a bead, a well or a stick. The captured target nucleic acid sequence can then be contacted with the detection probe under conditions of high stringency and the allele be detected.

One embodiment of the probe based technique based on TAQMAN technique. This is a method for measuring PCR product accumulation using a dual-labeled fluorogenic oligonucleotide probe called a TAQMAN® probe. This probe is composed of a short (ca. 20-25 bases) oligodeoxynucleotide that is labeled with two different flourescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two fluorophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity.

Other suitable methods include using mass spectrometry, single base extension, determining the Tm profile of a hybrid between a probe and a target nucleic acid sequence, using single strand conformation polymorphism, using single strand conformation polymorphism heteroduplex, using RFLP or RAPD, using HPLC, using sequencing of a target nucleic acid sequence from said biological sample.

Denaturing high-performance liquid chromatography (DHPLC) has been proven useful in human and animal genetic studies for detecting single nucleotide polymorphisms (SNPs). In contrary to most SNP detection methods that are currently in used, SNP detection by DHPLC is not based on a re-sequencing strategy that is expensive to implement, nor does it require gel-based genotyping procedures. Instead, SNP detection by DHPLC is based on resolving heteroduplex from homoduplex DNA fragments produced by PCR amplification using temperature-modulated heteroduplex analysis.

In connection with several of these methods there is a need for amplifying the amount of target nucleic acid in the biological sample isolated from the subject. Amplification may be performed by any known method including methods selected from the group consisting of polymerase chain reaction (PCR), Ligase Chain Reaction (LCR), Nucleic Acid Sequence-Based Amplification (NASBA), strand displacement amplification, rolling circle amplification, and T7-polymerase amplification.

More particularly, PCR-based amplification can be carried out using for example a primer pair comprising appropriate sequences selected from the sequences identified in Table 4 below:

| | | | | Primer |
|---|---|---|---|---|
| | | | | SEQ ID |
| Gene | SNP Rs ID No. | | Primer | NO |
| SLAM | rs3796504 | F | TGATCTCTAAGACCCTTTCC | 19 |
| | | R | CAGGTTATCATGATCAGCTC | 20 |
| | | snp | TCTATGCTAGTGTGACACTT | 21 |
| | rs2295612 | F | AAGTGCCTGGCTTCTTGAG | 22 |
| | | R | AAGGAAGAGTGACCAAACAC | 23 |
| | | snp | GCCAGGGAGAGAAACAGCAC | 24 |
| | ex 1b | F | AAGTGCCTGGCTTCTTGAG | 25 |
| | | R | AAGGAAGAGTGACCAAACAC | 26 |
| | | snp | CCCTTGGGATCCATCAGCCA | 27 |
| | rs12076998 | F | AAGTGCCTGGCTTCTTGAG | 28 |
| | | R | AAGGAAGAGTGACCAAACAC | 29 |
| | | snp | TGTGAGCAGCTGCCAGGCTC | 30 |
| | rs1000807 | F | AGTTATCTAAGTTCAGCTGTG | 31 |
| | | R | CAGAAGCAAGCTTCGTGTC | 32 |
| | | snp | GGGGGTGTGTAGTCACCTCG | 33 |
| | rs2295613 | F | AGTTATCTAAGTTCAGCTGTG | 34 |
| | | R | CAGAAGCAAGCTTCGTGTC | 35 |
| | | snp | CGGCTTTGGGCAGAAACATG | 36 |
| CD383 | prom 2 | F | ATACCAATCTGTGCACTGAC | 37 |
| | | R | GTTGACCCGCAAAAGGAAG | 38 |
| | | snp | ATGTTAACTGAAGTTACTTC | 39 |
| HRH1 | rs1171285 | F | TGTAACACTCCAATACTGCC | 40 |
| | | R | TATCCATAGACGGCAGTATC | 41 |
| | | snp | CTTTCTCAACCCATGTCTTA | 42 |
| | rs346074 | F | TGAAGGTCTTCTCCATGATG | 43 |
| | | R | TCTGGTAATTGCCAAATGATG | 44 |
| | | snp | TAATCAGATAGTACAGTAAT | 45 |
| | rs901865 | F | CATCTTGTCTTCTAAGAGGC | 46 |
| | | R | CATACAACTCCAGTCTGATG | 47 |
| | | snp | AGGGAGTGAGCCATAACTGG | 48 |
| | rs2067470 | F | ACAGTATGTATCTGGGTTGC | 49 |
| | | R | TTGAAGTTCTCATTGCACAAG | 50 |
| | | snp | ACTGTTGCAATGAACATT | 51 |
| IL2 | rs2069763 | F | GTTCCCTATCACTCTTTAAT | 52 |
| | | R | TTTCATATTACTTTGAATTTT | 53 |
| | | | ATT | |
| | | snp | AAAATCATCTGTAAATCCAG | 54 |
| | rs2069762 | F | TGTACATAGACATTAAGAGAC | 55 |
| | | R | AGCCCACACTTAGGTGATAGC | 56 |
| | | snp | CACATGTTCAGTGTAGTTTTA | 57 |
| TLR7 | rs179008 | F | CAAAAGAGAGGCAGCAAATG | 58 |
| | | R | CACAGTTGCATGTGAAATCG | 59 |
| | | snp | AATGTGGACACTGAAGAGAC | 60 |
| | rs5743781 | F | AAAGCCTGAAAATTCTGCGG | 61 |
| | | R | TACTTAGATCCAAGGTCTGC | 62 |
| | | snp | AACTTTCTACAGAAGTTCTG | 63 |
| | rs864058 | F | TTGCGATATCTGGATCTCAG | 64 |
| | | R | TGACTTGCTGTCATCATCAC | 65 |
| | | snp | GTCTGGTGGGTTAACCATAC | 66 |
| TLR8 | rs5741883 | F | GTCACCATTCTGCTTGGTTG | 67 |
| | | R | ACAAGTTTCTGAGACAGCAC | 68 |
| | | snp | CCTCCTCCAGCACCTGGC | 69 |
| | rs3764879 | F | TGTGTGTCTGATTTGGGTTG | 70 |
| | | R | TTCTAGGCTCACACCATTTG | 71 |
| | | snp | CTTCTGTAAAACACACGCTA | 72 |
| | rs3764880 | F | TGTGTGTCTGATTTGGGTTG | 73 |
| | | R | TTCTAGGCTCACACCATTTG | 74 |
| | | snp | AAAATTAGAACAACAGAAAC | 75 |
| | rs5744077 | F | CATTCTGGACCTAATCTGATG | 76 |
| | | R | TATCAGACAGGTCTAGTTCTG | 77 |
| | | snp | CAGGAAAATGCAGGTCAGCA | 78 |
| | rs2159377 | F | ATGTGACAGAACTAGACCTG | 79 |
| | | R | TATAAGTCTTGAAATGCCCTC | 80 |
| | | snp | AATGGCTTGAATATCACAGA | 81 |
| | rs2407992 | F | CTATTTCAGATTAGCAGGCG | 82 |
| | | R | AAACTGCTGGAGTAATGTCC | 83 |
| | | snp | GATTTATCCCTTAATAGGCT | 84 |
| TLR10 | rs11466657 | F | AATTGCTCATGGCCAGAAAC | 85 |
| | | R | AGGGTATTCACAGGTGTATG | 86 |
| | | snp | GGCCTTACGAGAACTAAATA | 87 |
| | rs11466655 | F | GGAGCATGTACATTTCAGAG | 88 |
| | | R | ACCTGAAGACAGAATCAGAC | 89 |
| | | snp | GAAAACTCTCATTTTGAATG | 90 |
| | rs11096955 | F | GGAGCATGTACATTTCAGAG | 91 |
| | | R | ACCTGAAGACAGAATCAGAC | 92 |
| | | snp | TTTCAAGTGAGGCAGTTGGA | 93 |
| | rs11096956 | F | GGAGCATGTACATTTCAGAG | 94 |
| | | R | ACCTGAAGACAGAATCAGAC | 95 |
| | | snp | ATGCCACACATGCTTTTCCC | 96 |
| | rs11096957 | F | CTGCCCATCTTAAACACAAC | 97 |
| | | R | ATTGTCAGGTTTTCTATGTCC | 98 |
| | | snp | AACGAAATCTTAGTTTAGAA | 99 |
| | none | F | AACCTTACTCCAACCTCTTG | 100 |
| | | R | GAGATCCAGCTGTTGAATTC | 101 |
| | | snp | CATCATTCATATGAGGAAAT | 102 |
| | rs11466645 | F | GTTTCTGGCAGAATAGGTAC | 103 |
| | | R | AGATAGGCATGGTGTTAGTC | 104 |
| | | snp | TCCCAAAGTCCTCAGAATTC | 105 |
| | rs11466642 | F | GTTTCTGGCAGAATAGGTAC | 106 |
| | | R | AGATAGGCATGGTGTTAGTC | 107 |
| | | snp | CAACTACCTCTGTTCTAC | 108 |
| CD86 | ex 5 | F | TGCTATTCCCTCCTAGATAC | 109 |
| | | R | TTGGATGATCTGCCTTAAGC | 110 |
| SFRS8 | rs1051219 | F | GACCGTGGCAGCCATGTATTA | 111 |
| | | R | GGTCGTCACTCCAGGGGAGT | 112 |
| | Probe 1 | (A) | Fam-ccctcccggaatcgacgt | 113 |
| | | | gact-Tamra | |
| | Probe 2 | (G) | Joe-cccctcccggaatcgat | 114 |
| | | | gtgact-Tamra | |
| | rs1051233 | F | CTGGAAGATCGCCTCGCA | 115 |
| | | R | TCTGCTTCCGGCAGAGGAT | 116 |
| | Probe 1 | (A) | Fam-tgcccgggaaaag | 117 |
| | | | ctggcc-Tamra | |
| | Probe 2 | (G) | Joe-tgcccgggaaaag | 118 |
| | | | ctcgcc-Tamra | |
| | rs1379049 | F | CGCCACCCTGGGCAGA | 119 |
| | | R | TGCTGCAGCCTGCCACAT | 120 |
| | Probe 1 | (A) | Fam-cctccgcgtccctcacc | 121 |
| | | | atg-Tamra | |
| | Probe 2 | (G) | Vic-agcctccgcgcccctca | 122 |
| | | | c-Tamra | |
| | rs378288 | F | TGAGTCAAACCATGTCCTGCC | 123 |
| | | R | CGTGGTGTCCATGTTAGTGGAG | 124 |
| | Probe 1 | (A) | Fam-gcctagtcactaaaa- | 125 |
| | | | MGB | |
| | Probe 2 | (G) | Vic-gcctagtcactagaac- | 126 |
| | | | MGB | |

F—forward PCR primer
R—reversed PCR primer
snp - primers for the single base extension detection method
Probe 1 and 2 - TAQMAN ® probes

One of the primers may comprise a moiety for subsequent immobilisation of the amplified fragments.

It is understood that the primers identified above may also be used as probes for determining the polymorphisms of the invention in a nucleic acid sequence using any of the methods known in the art and featured above.

To the extent that the polymorphisms as defined in the present invention are present in DNA sequences transcribed as mRNA transcripts these transcripts constitute a suitable target sequence for detection of the polymorphisms. Commercial protocols are available for isolation of total mRNA. Through the use of suitable primers the target mRNA can be amplified and the presence or absence of polymorphisms be detected with any of the techniques described above for detection of polymorphisms in a DNA sequence.

### 3.2 Proteins

Genetic polymorphism can also be detected as a polymorphism of a protein product of the gene, or a change in a biological response, e.g. immune response, where the protein is involved.

For example, the genetic polymorphisms according to the present invention may influence the co-stimulatory signalling in T cell activation or are linked to polymorphisms having this physiological effect, the diagnosis may also be carried out by measuring the relative amount of cytokines expressed downstream from the co-stimulatory signal in immune response pathway in a biological sample from a subject suffering from said diseases.

More particularly the signalling may be measured by measuring the relative amount of cytokines selected from the group comprising IL4, IL5, IL10, and IL13. It is expected that the result of a predisposing allele of a polymorphism as defined in the present invention is that the relative amount of IL4, IL5 and IL13 is increased and the relative level of IL10 decreases.

The polymorphism located for example in the CD86 gene, SLAMF1, TLR7, TLR10 or CD83 genes may also be detected by isolating a variant protein from a biological sample and determining the presence or absence of the mutated residue (according to Table 2 above) by sequencing said protein, or determining the presence or absence of another polymorphic amino acid of a variant potein by sequencing a transcriptional peroduct of the corresponding gene. The polymorphism of any of the variant proteins of the invention may be detected likewise.

Determining the polymorphism of the SFRS8 gene may be for example related to determining isoform profile or activity of CD45 protein.

The presence or absence of the valine residue in the mutated CD86 protein may for example be detected by isolating the protein from a biological sample and determining the binding affinity towards the CD86 and/or the CTLA4 receptor relative to the binding affinity of wildtype CD86 protein. Assays for determining this binding affinity are known e.g. from Jeannin et al 2000 (Immunity, vol 13:303-312). Another example of a competitive binding assay is the following based on competitive binding between biotinylated wildtype CD86 and mutant CD86.

The ability of CTLA4 or CD28 to bind to CD86 is assessed in a competitive binding ELISA assay as follows. Purified recombinant CTLA4 (20 μg/ml in PBS) is bound to a Costar EIA/RIA 96 well microtiter dish (Costar Corp, Cambridge Mass., USA) in 50 μL overnight at room temperature. The wells are washed three times with 200 μL of PBS and the unbound sites blocked by the addition of 1% BSA in PBS (200 PI/well) for 1 hour at room temperature. The wells are washed as above. Biotinylated CD86 (1 μg/ml serially diluted in twofold steps to 15.6 ng/mL; 50 μL) is added to each well and incubated for 2.5 hours at room temperature. The wells are washed as above. The bound biotinylated CD86 is detected by the addition of 50 μl/well of a 1:2000 dilution of streptavidin-HRP (Pierce Chemical Co., Rockford, Ill.) for 30 minutes at room temperature. The wells are washed as above and 50 μL of ABTS (Zymed, Calif.) added and the developing blue colour monitored at 405 nm after 30 min. The ability of unlabelled CD86 to compete with biotinylated CD86, respectively, is assessed by mixing varying amounts of the competing protein with a quantity of biotinylated CD86 shown to be non-saturating (i.e., 70 ng/mL; 1.5 nM) and performing the binding assays as described above. A reduction in the signal (Abs 405 nm) expected for biotinylated CD86 indicates a competition for binding to immobilised CTLA4.

Polymorphism of a gene of the invention may also be identified by using an antibody raised against a variant protein expressed by the polymorphic gene, e.g. a variant protein of Table 2 above. By using an antibody which is able to recognise an epitope comprising a region of the variant protein comprising a polymorphism corresponding to the polymorphism of the gene it is possible to determine a predisposition of an individual to an immune related disease of the invention without screening the genetic material. Thus, an antibody which is capable of specifically binding to an epitope comprising a polymorphism of the invention is also in the scope of the invention.

Antibodies within the invention include polyclonal antibodies, monoclonal antibodies, humanized or chimeric antibodies, single chain antibodies, Fab′ fragments, F(ab′)₂ fragments, and molecules produced using a Fab expression library, and antibodies or fragments produced by phage display techniques.

Polyclonal and/or monoclonal antibodies, which are homogeneous populations of antibodies to a particular antigen, can be prepared using variant proteins (natural or recombinant) or fragment of these proteins which contain the polymorphism by standard technologies.

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. (In the case of chckens, the immunoglobulin class can also be IgY.) The hybridoma producing the mAb of this invention may be cultivated in vitro or in vivo. The ability to produce high titers of mAbs in vivo makes this the presently preferred method of production, but in some cases, in vitro production will be preferred to avoid introducing cancer cells into live animals, for example, in cases where the presence of normal immunoglobulins coming from the acitis fluids are unwanted, or in cases involving ethical considerations.

Once produced, polyclonal, monoclonal, or phage-derived antibodies are tested for specific recognition of the above described epitope by Western blot or immunoprecipitation in samples containing the polypeptides comprising the binding site or fragments thereof, e.g., as described in Ausubel et al., supra. Antibodies that specifically recognise a polymorphism of the variant protein are useful in the invention.

Such antibodies can be used in an immunoassay to monitor the spectrum of the expressed protein of interst or a level of expression a variant protein in a sample collected from an individual. An antibody with is capable to inhibit an immune related activity of a variant protein is of a particular interest as a candidate compound for the treatment of an immune related disease of the invention.

The antibody may also be used in a screening assay for measuring activity of a polymorphic gene of the invention, for example as a part of a diagnostic assay. Depending on the detection technique the antibody may be coupled to a compound comprising a detectable marker. The markers or labels may be selected from any markers and labels known in the art. The antibody may also be used for determining the concentration of a substance comprising an epitope or epitope in a solution of said substance or said epitope. A wide spectrum of detection and labelling techniques is available now in the art and the techniques may therefore be selected depending on skills of the artisan practising the antibodies or on the purpose of using thereof.

In addition, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778, 4,946,778, and 4,704,692) can be adapted to produce single chain antibodies against a variant protein of the invention or a fragment thereof comprising a polymorphim. Single chain antibodies are formed by linking the heavy and light chain fragments of the Fv region via an amino acid bridge, resulting in a single chain polypeptide.

Antibody fragments that recognise and bind to specific epitopes can be generated by known techniques. For example, such fragments include but are not limited to F(ab′)₂ fragments that can be produced by pepsin digestion of the antibody molecule, and Fab′ fragments that can be generated by reducing the disulfide bridges of F(ab′)₂ fragments. Alternatively, Fab′ expression libraries can be constructed (Huse et al., Science, 246:1275, 1989) to allow rapid and easy identification of monoclonal Fab′ fragments with the desired specificity.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

Thus, isolated/identified variant proteins expressed by any of the other polymorphic genes of the invention may be used as alternative diagnostic markers of the genetic polymorphism associated with a predisposition to an immune related disease of the invention.

### 4. Biological Sample

The biological sample used in the present invention may be any suitable biological sample comprising genetic material and/or proteins involved in induction of the immune response as described previously. In a preferred embodiment the sample is a blood sample, a tissue sample, a secretion sample, semen, ovum, hairs, nails, tears, and urine. The most convenient sample type is a blood sample.

### 5. Isolated Oligonucleotides

In one aspect the invention relates to an isolated oligonucleotide comprising at least 10 contiguous nucleotides being 100% identical to a subsequence of the genes of the invention comprising or adjacent to a polymorphism or mutation being correlated to an immune-related disease, or being 100% identical to a subsequence of the human genome which is in linkage disequilibrium with any of the genes of the invention comprising or adjacent to a polymorphism or mutation being correlated to an immune-related disease. As explained in the summary, such probes may be used for detecting the presence of a polymorphism of interest and/or they may constitute part of a primer pair and/or they may form part of a gene therapy vector used for treating the immune-related diseases.

Preferably the isolated oligonucleotide comprises at least 10 contiguous bases of a sequence identified as SEQ ID NOs: 10-18 or the corresponding complementary strand, or a strand sharing at least 90% sequence identity more preferably at least 95% sequence identity with SEQ ID NOs: 10-18 or a complementary strand thereof, said isolated oligonucleotide comprising a polymorphism of the invention.

Further preferred isolated oligonucleotide may comprise at least 10 contiguous bases of any of the sequences identified as SEQ ID NOS: 1-9 or the corresponding complementary strand thereof, or a strand sharing at least 90% sequence identity more preferably at least 95% sequence identity with any of the SEQ ID NOS: 1-9 or a complementary strand thereof, said isolated oligonucleotide comprising a polymorphism of the invention.

These particular oligonucleotides may be used as probes for assessing the polymorphisms in the human SFRS8, CD83, SLAMF1, CD86, HRH1, IL2, TLR7, TLR8 or TLR10 genes which are strongly correlated with immune-related diseases of the invention.

The length of the isolated oligonucleotide depends on the purpose. When being used for amplification from a sample of genomic DNA, the length of the primers should be at least 15 and more preferably even longer to ensure specific amplification of the desired target nucleotide sequence. When being used for amplification from mRNA the length of the primers can be shorter while still ensuring specific amplification. In one particular embodiment one of the pair of primers may be an allele specific primer in which case amplification only occurs if the specific allele is present in the sample. When the isolated oligonucleotides are used as hybridisation probes for detection, the length is preferably in the range of 10-15 nucleotides. This is enough to ensure specific hybridisation in a sample with an amplified target nucleic acid sequence. When using nucleotides which bind stronger than DNA (e.g. LNA and/or PNA), the length of the probe can be somewhat shorter, e.g. down to 7-8 bases.

The length may be at least 15 contiguous nucleotides, such as at least 20 nucleotides. An upper limit preferably determines the maximum length of the isolated oligonucleotide. Accordingly, the isolated oligonucleotide may be less than 1000 nucleotides, more preferably less than 500 nucleotides, more preferably less than 100 nucleotides, such as less than 75 nucleotides, for example less than 50 nucleotides, such as less than 40 nucleotides, for example less than 30 nucleotides, such as less than 20 nucleotides.

The isolated oligonucleotide may comprise from 10 to 50 nucleotides, such as from 10 to 15, from 15 to 20, from 20 to 25, or comprising from 20 to 30 nucleotides, or from 15 to 25 nucleotides.

Depending on the use the polymorphism may be located in the centre of the nucleic acid sequence, in the 5′ end of the nucleic acid sequence, or in the 3′ end of the nucleic acid sequence.

For detection based on single base extension the sequence of the oligonucleotide is adjacent to the mutation/polymorphism, either in the 3′ or 5′ direction.

The isolated oligonucleotide sequence may be complementary to a sub-sequence of the coding strand of a target nucleotide sequence or to a sub-sequence to the non-coding strand of a target nucleotide sequence as the polymorphism may be assessed with similar efficiency in the coding and the non-coding strand.

The isolated oligonucleotide sequence may be made from RNA, DNA, LNA, PNA monomers or from chemically modified nucleotides capable of hybridising to a target nucleic acid sequence. The oligonucleotides may also be made from mixtures of said monomers.

### 6. Kits

In one aspect there is provided a kit for predicting the risk of a subject for developing immune related diseases or for other diagnostic and classification purposes of immune related diseases comprising at least one probe comprising a nucleic acid sequence as defined in the previous section.

In one embodiment the probe is linked to a detectable label.

In another embodiment based on single nucleotide extension the kit further comprises at least one nucleotide monomer labelled with a detectable label, a polymerase and suitable buffers and reagents.

The kit preferably also comprises set of primers for amplifying a region comprising at least two of the identified above polymorphisms in any of the genes selected from the SFRS8, CD83, SLAMF1, CD86, HRH1, IL2, TLR7, TLR8 and TLR10 genes or transcriptional products of said genes, or the corresponding complementary strands.

The primers preferably are at least 15 bases long and may be coupled to an entity suitable for subsequent immobilisation.

A kit may also comprise an antibody capable of recognising the polumorphism of the invention.

### 7. Immune-Related Disease

The invention related to association of two or more polymorphisms in the above genes, or association of at least one of the above identified SNPs with a predisposition to an immune related disease. In particular, the invention relates to a predisposition to a disease selected from asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

Allergic conditions in connection with infectious diseases, autoimmune diseases, graft/host incompatibilities are also in the scope of the invention.

As follows from the results of haplotype analysis presented in FIGS. 1-22 of the present application, the association of certain diseases with the presence of different haplotypes of SNPs described herein is not identical. Table 5 below shows selected but non-limited examples of the association of certain SNPs with particular immune-related diseases.

TABLE 5

| | | | | Ast- | | AD- | | Rh- | |
|---|---|---|---|---|---|---|---|---|---|
| Gene | SNP | Rast | Ast | rast | AD | rast | Rh | rast | Skin |
| TLR8 | rs5741883 | | | | | | | 0.034 | |
| | rs2407992 | 0.043 | 0.001 | 0.011 | | 0.034 | | | |
| TLR10 | rs11466657 | | | | | 0.025 | | | |
| | rs11096955 | | | | | 0.030 | | | |
| CD83 | prom 2 | 0.023 | 0.0094 | | | 0.025 | 0.014 | | |
| HRH1 | rs1171285 | | 0.038 | | 0.034 | 0.027 | | | 0.030 |
| | rs346074 | 0.016 | 0.010 | 0.016 | 0.0087 | 0.0084 | 0.033 | 0.041 | 0.0069 |
| | rs901865 | | 0.033 | | | | | | 0.020 |
| IL2 | rs2069763 | | | | 0.030 | 0.027 | | | |
| | rs2069762 | 0.018 | 0.018 | 0.023 | 0.027 | 0.043 | 0.012 | | |
| SLAMF1 | rs3796504 | | | | | | | 0.048 | |
| | rs12076998 | 0.009 | 0.00068 | 0.0094 | 0.029 | 0.028 | 0.0080 | 0.013 | 0.0035 |
| | rs1000807 | | 0.025 | | | | | | |
| | rs2295613 | | | | | 0.020 | | | |
| TLRL7 | rs179008 | 0.023 | | 0.041 | | | 0.013 | 0.0039 | |
| | rs5743781 | 0.025 | | | | | | | |
| SFRS8 | rs755437 | | 0.017 | 0.036 | 0.0018 | | | 0.0006 | |
| | | | 0.0079 | | | | | | |
| | rs1051219 | | | 0.0067 | | | | 0.0063 | |
| | rs1051233 | | | 0.014 | 0.0088 | | | 0.013 | |
| | rs1379049 | | | | 0.28 | | | | |
| | rs3782288 | | | | 0.28 | | | | |

The association is expressed as p-values obtained by the transmission disequilibrium test (TDT).

### Ast: Asthma

AD: Atopic dermatitis
Rast: Elevated specific serum IgE

### Rh Rhinitis

Ast-rast: Asthma and elevated specific serum IgE
Rh-rast: Rhinitis and elevated specific serum IgE
AD-rast: Atopic dermatitis and elevated specific serum IgE
Skin: Positive skin test

According to the invention an association of a SNP of table 5 with a particular disease indicates the association of expression of a particular allele of said SNP with a predisposition to said disease. The protective/risky alleles of the above SNP are indicated in Table 6 below.

TABLE 6

| | |
|---|---|
| SEQ | Allele |

| Gene | ID NO | SNP No | protective | risky |
|---|---|---|---|---|
| SLAMF1 | 1 | rs3796504 | A | C |
| | | rs12076998 | T | C |
| | | rs1000807 | G | T |
| | | rs2295613 | T | C |
| CD86 | 2 | ex 5 | G | A |
| CD83 | 3 | prom 2 | T | C |
| HRH1 | 4 | rs1171285 | C | A |
| | | rs346074 | A | G |
| | | rs901865 | A | G |
| IL2 | 5 | rs2069763 | C | A |
| | | rs2069762 | G | T |
| TLR7 | 6 | rs179008 | T | A |
| | | rs5743781 | A | G |
| TLR8 | 7 | rs5741883 | A | G |
| | | rs2407992 | C | G |
| TLR10 | 8 | rs11466657 | T | C |
| | | rs11096955 | C | A |
| SFRS8 | 9 | rs755437 | C | T |
| | | rs1051219 | C | T |
| | | rs1051233 | G | C |
| | | rs1379049 | A | G |
| | | rs3782288 | G | A |

According to the invention individuals carrying the protective alleles of SNPs identified in the table are less likely to develop an immune-related disease of the invention. In contrary, the presence of the risky allele is indicative of a predisposition to an immune-related disease.

Thus, in one embodiment the invention relates to a method for determining a predisposition of an individual for asthma, said method comprising determining at least one SNP selected from the SNPs identified herein as prom2, rs2407992, rs1171285, rs346074, rs901865, rs2069762, rs12076998, rs1000807 and rs755437. In another embodiment the determining a predisposition of an individual for asthma comprises determining an SNP selected from the group consisting of SNPs identified herein as prom2, rs2407992, rs12076998, rs1000807 and rs755437.

In another embodiment the invention relates to a method for determining a predisposition of an individual to rhinitis, said method comprising determining at least one SNP selected from the SNPs identified herein as prom 2, rs346074, rs2069762, rs12076998, rs179008, rs755437, rs1051219, rs1051233. In another embodiment the determining a predisposition to rhinitis comprises determining a SNP selected from the SNPs identified as prom 2, rs346074, rs12076998, rs179008. In still another embodiment the determining a predisposition to rhinitis may comprise determining an SNP selected from the group consisting of SNPs having the Ref. Id: rs755437, rs1051219, rs1051233

In still another embodiment, the invention relates to a method for determining a predisposition of an individual to atopic dermatitis, said method comprising determining at least one SNP selected from the SNPs identified above as rs1171285, rs346074, rs2069763, rs2069762, rs12076998. In another embodiment the determining a predisposition of an individual to atopic dermatitis comprises determining an SNP selected from the group consisting of SNPs having the Ref. Id: rs1171285, rs12076998. In still another embodiment the determining a predisposition to atopic dermatitis may comprise determining an SNP selected from the group consisting of SNPs identified as rs755437, rs1051233, rs1379049, rs3782288.

In yet another embodiment, the invention relates to a method for determining a predisposition of an individual to the elevated level of specific serum IgE, said method comprising determining at least one SNP selected from the SNPs identified herein as prom 2, rs2407992, rs346074, rs2069762, rs12076998, rs179008, rs5743781.

In yet another embodiment, the invention relates to a method for determining a predisposition of an individual to the positive skin test, said method comprising determining at least one SNP selected from the SNPs identified herein as rs1171285, rs346074, rs901865, rs12076998.

Other embodiments of the invention concern methods for determining a predisposition of an individual to
i) Asthma and elevated specific serum IgE, said method comprising determining at least one SNP selected from the SNPs identified above as rs2407992, rs346074, rs2069762, rs12076998, rs179008, rs755437, rs1051219, rs1051233;
ii) Rhinitis and elevated specific serum IgE, said method comprising determining at least one SNP selected from the SNPs identified above as rs346074, rs12076998, rs179008;
iii) Atopic dermatitis and elevated specific serum IgE, said method comprising determining at least ove SNP selected from the SNPs identified above as rs2407992, rs11466657, rs11096955, prom 2, rs1171285, rs346074, rs2069763, rs2069762, rs12076998, rs2295613, rs755437, rs1051219, rs1051233.

In some embodiments a method for determining a predisposition to any immune related disease of the invention may concern the determining two or more of the SNPs identified in Table 5. However, in some embodiments the determining a single of the above SNPs may be sufficient for the determining a predisposition to the disease.

### 8. Medical Treatment

The present invention relates to a SFRS8, CD83, SLAMF1, CD86, HRH1, IL2, TLR7, TLR8 and/or TLR10 gene associated disorder in particular immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

Having identified a group of subjects having a polymorphism as described in the present invention, the invention also relates to the use of compounds directed to decreasing or modulating the effect of the polymorphism for the preparation of a medicament for the treatment of immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema in said subjects.

The compounds that bind to a SFRS8, CD83, SLAMF1, CD86, HRH1, IL2, TLR7, TLR8 and/or TLR10 gene product, intracellular proteins or portions of proteins that interact with a SFRS8, CD83, SLAMF1, CD86, HRH1, IL2, TLR7, TLR8 and/or TLR10 gene product, compounds that interfere with the interaction of a SFRS8, CD83, SLAMF1, CD86, HRH1, IL2, TLR7, TLR8 and/or TLR10 gene product with intracellular proteins and compounds that modulate the activity of the SFRS8, CD83, SLAMF1, CD86, HRH1, IL2, TLR7, TLR8 and TLR10 genes (i.e. modulate the level of the SFRS8, CD83, SLAMF1, CD86, HRH1, IL2, TLR7, TLR8 and TLR10 gene expression and/or modulate the level of the SFRS8, CD83, SLAMF1, CD86, HRH1, IL2, TLR7, TLR8 and TLR10 gene product activity) are considered to be good candidates for the manufacture of a medicament for treatment of a SFRS8, CD83, SLAMF1, CD86, HRH1, IL2, TLR7, TLR8 and/or TLR10 gene associated disorder.

It is to be understood that compounds that considered by the invention to be good candidates for the manufacture of a medicament for treatment of a a SFRS8, CD83, SLAMF1, CD86, HRH1, IL2, TLR7, TLR8 and/or TLR10 gene associated disorder described in the application are the compounds that can modulate the level of the polymorphic SFRS8, CD83, SLAMF1, CD86, HRH1, IL2, TLR7, TLR8 and TLR10 gene expression and/or modulate the level of the polymorphic SFRS8, CD83, SLAMF1, CD86, HRH1, IL2, TLR7, TLR8 and TLR10 gene product activity, wherein the polymorphism is as the described above.

Assays may additionally be utilized that identify compounds that bind to the SFRS8, CD83, SLAMF1, CD86, HRH1, IL2, TLR7, TLR8 and/or TLR10 gene regulatory sequences (e.g., promoter sequences; see e.g., Platt, 1994, J. Biol. Chem. 269, 28558-28562), and that may modulate the level of SFRS8, CD83, SLAMF1, CD86, HRH1, IL2, TLR7, TLR8 and/or TLR10 gene expression. Compounds may include, but are not limited to, small organic molecules, such as ones that are able to gain entry into an appropriate cell and affect expression of the SFRS8, CD83, SLAMF1, CD86, HRH1, IL2, TLR7, TLR8 and/or TLR10 gene or some other gene involved in a SFRS8, CD83, SLAMF1, CD86, HRH1, IL2, TLR7, TLR8 and/or TLR10 gene dependent regulatory pathway (such as for example the genes described in the application), or intracellular proteins. Such intracellular proteins may for example be involved in the control and/or regulation of the immune response to an allergen. Further, among these compounds are compounds that affect the level of SFRS8, CD83, SLAMF1, CD86, HRH1, IL2, TLR7, TLR8 and/or TLR10 gene expression and/or the SFRS8, CD83, SLAMF1, CD86, HRH1, IL2, TLR7, TLR8 and/or TLR10 gene product activity and that can be used as medicaments in the therapeutic treatment of the SFRS8, CD83, SLAMF1, CD86, HRH1, IL2, TLR7, TLR8 and/or TLR10 gene associated disorders, for example an immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

Compounds may include, but are not limited to, peptides such as, for example, soluble peptides, including but not limited to, Ig-tailed fusion peptides, and members of random peptide libraries; (see, e.g., Lam, et al., 1991, Nature 354, 82-84; Houghten, et al., 1991, Nature 354, 84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72, 767-778), anti-bodies (including, but not limited to, polyclonal, monoclonal, humanized, antiidiotypic, chimeric or single chain antibodies, and FAb, F(ab′).sub.2 and Fab expression library fragments, and epitope-binding fragments thereof), and small organic or inorganic molecules. Such compounds may further comprise compounds, in particular drugs or members of classes or families of drugs, known to ameliorate or exacerbate the symptoms of immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, such as anti-inflammatory drugs, glucocorticoids, antihistamines, allergen-specific immuno preparates, sympatomimetics, anti-astma compounds, such as alpha1, alpha 2, beta1 and beta2 antagonists, leukotrien receptor antagonist, such as montelukast, parasympatolytics, such as ipratropium, theophyllin and theophyllamin, croglicat, nedocromil and methorexat. Many of these drugs can be or have been used in combination.

Compounds identified via assays such as those described herein may be useful, for example, in elaborating the biological function of the SFRS8, CD83, SLAMF1, CD86, HRH1, IL2, TLR7, TLR8 and TLR10 gene products, and for ameliorating the SFRS8, CD83, SLAMF1, CD86, HRH1, IL2, TLR7, TLR8 and TLR10 gene associated disorders, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

### Inhibitory Antisense, Ribozyme and Triple Helix Approaches

In another embodiment, symptoms of certain immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, may be ameliorated by decreasing the level of SFRS8, CD83, SLAMF1, CD86, HRH1, IL2, TLR7, TLR8 and/or TLR10 gene expression and/or the SFRS8, CD83, SLAMF1, CD86, HRH1, IL2, TLR7, TLR8 and/or TLR10 gene product activity by using the SFRS8, CD83, SLAMF1, CD86, HRH1, IL2, TLR7, TLR8 and/or TLR10 gene derived nucleotide sequences in conjunction with well-known antisense, gene “knockout,” ribozyme and/or triple helix methods to decrease the level of SFRS8, CD83, SLAMF1, CD86, HRH1, IL2, TLR7, TLR8 and/or TLR10 gene expression. Among the compounds that may exhibit the ability to modulate the activity, expression of the SFRS8, CD83, SLAMF1, CD86, HRH1, IL2, TLR7, TLR8 and/or TLR10 gene and/or synthesis the gene products, including the ability to ameliorate the symptoms of a SFRS8, CD83, SLAMF1, CD86, HRH1, IL2, TLR7, TLR8 and/or TLR10 gene disorder, are antisense, ribozyme, and triple helix molecules. Such molecules may be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targetted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of a RNA sequence, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the SFRS8, CD83, SLAMF1, CD86, HRH1, IL2, TLR7, TLR8 and/or TLR10 gene could be used in an antisense approach to inhibit translation of endogenous SFRS8, CD83, SLAMF1, CD86, HRH1, IL2, TLR7, TLR8 and/or TLR10 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N²-carboxypropyl) uracil, (acp3)_{w}, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a form acetal or analog thereof.

In yet another embodiment, the antisense oligonucleotide is an .alpha.-anomeric oligonucleotide. An alpha.-anomeric oligonucleotide forms specific double-stranded hybrids with complementary RNA in which, contrary to the usual .beta.-units, the strands run parallel to each other (Gautier, et al., 1987, Nucl. Acids Res. 15, 6625-6641). The oligonucleotide is a 2′-O-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred. For example, antisense oligonucleotides having the following sequences can be utilized in accordance with the invention:

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290, 304-310), the promoter contained in the 31 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ri-bozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

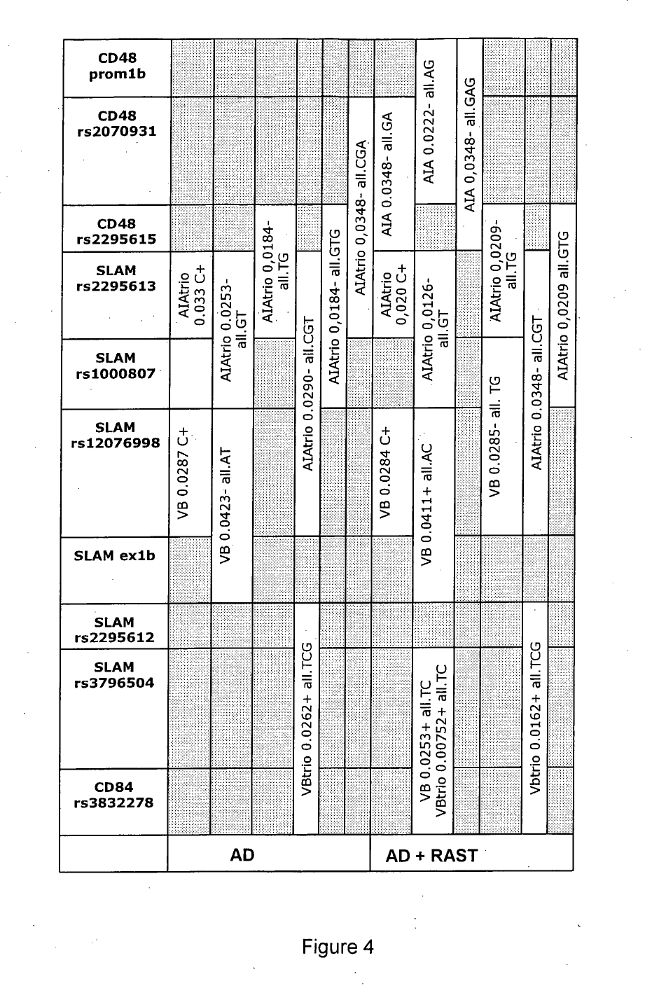

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially Figure. 4, page 833) and in Haseloff and Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts. For example, hammerhead ribozymes having the following sequences can be utilized. The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence where after cleavage of the target RNA takes place.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol 11 promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC.sup.+triplets across the three associated strands of the resulting triple helix. The pyrimidinerich molecules provide base complementarity to a purine-rich region of a single strand of the duplex in a parallel orientation to that strand. In addition, nucleic acid molecules may be chosen that are purine-rich, for example, that contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.9.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense

RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### Gene Therapy

Having identified polymorphism(s) as the cause of a disease it is also rendered possible with the present invention to provide a genetic therapy for subjects being diagnosed as having a predisposition according to the invention, said therapy comprising administering to said subject a therapeutically effective amount of a gene therapy vector. The gene therapy vectors carry the protective allele of the genes. The protective allele means in the present content that expression of this allele in an individual indicates no predisposition to an immune related disease of the invention. Selected, but not limited examples of protective/risky alleles of the nucleotides at positions associated with a predisposition to an immune related disease are shown in Table 5.

Having discovered the SFRS8, CD83, SLAMF1, CD86, HRH1, IL2, TLR7, TLR8 and TLR10 genes as etiological factors in immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, the inventors also provide methods for gene therapy and gene therapy vectors for use in subjects irrespective of whether they carry any of the susceptibility or protective alleles/haplotypes described in the present invention. In particular the invention relates to a gene therapy vector comprising i) a DNA sequence selected from the sequences identified as SEQ ID NO 1-9, or a fragment thereof, or ii) a DNA sequence selected from the sequences identified as SEQ ID NOs: 10-18, or a fragment of said DNA sequence, wherein the DNA sequence or the fragment thereof comprises the protective allele of an SNP selected from the SNPs identified as rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233, rs1379049.

There are various different methods of gene therapy for the subjects defined in the present invention.

The first two are based on activation of the repair system of the cells by introducing into those cells a gene therapy vector which causes “correction” of the polymorphism by presenting the repair mechanism with a template for carrying out the correction. One such type includes the RNA/DNA chimeraplast, said chimeraplast being capable of correcting the polymorphism in cells of said subject. Examples of the design of such chimeraplasts can be found in e.g. U.S. Pat. No. 5,760,012; U.S. Pat. No. 5,888,983; U.S. Pat. No. 5,731,181; U.S. Pat. No. 6,010,970; U.S. Pat. No. 6,211,351.

The second method is based on application of single stranded oligonucleotides, wherein the terminal nucleotides is protected from degradation by using 3′ and 5′ phosphorothioat-linkage of the monomers. This gene therapy vector is also capable of “correcting” the polymorphism by replacing one nucleotide with another.

These first two types of gene therapy vectors comprise a small sequence (less than 50 bases) which overlaps with the polymorphism in question. Suitable sequences for this purpose are genomic sequences located around the polymorphism.

Other types of gene therapy include the use of retrovirus (RNA-virus). Retrovirus can be used to target many cells and integrate stably into the genome. Adenovirus and adeno-associated virus can also be used. A suitable retrovirus or adenovirus for this purpose comprises an expression construct with the wildtype gene under the control of the wildtype promoter or a constitutive promoter or a regulatable promoter such as a repressible and/or inducible promoter or a promoter comprising both repressible and inducible elements.

A further group of gene therapy vectors includes vectors comprising interfering RNA (RNAi) for catalytic breakdown of mRNA carrying the polymorphism. RNAi can be used for lowering the expression of a given gene for a relatively short period of time. In particular these RNAi oligos may be used for therapy for both subjects carrying a susceptibility allele as described in the present invention as well as for subjects which do not carry such an allele.

Interfering RNA (“RNAi”) is double stranded RNA that results in catalytic degradation of specific mRNAs, and can also be used to lower gene expression.

Described below are methods and compositions whereby a SFRS8, CD83, SLAMF1, CD86, HRH1, IL2, TLR7, TLR8 and/or TLR10 gene disorder, in particular immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, may be treated.

With respect to an increase in the level of normal SFRS8, CD83, SLAMF1, CD86, HRH1, IL2, TLR7, TLR8 and TLR10 gene expression and/or SFRS8, CD83, SLAMF1, CD86, HRH1, IL2, TLR7, TLR8 and TLR10GENE product activity, the SFRS8, CD83, SLAMF1, CD86, HRH1, IL2, TLR7, TLR8 and TLR10 gene derived nucleotide sequences, for example, be utilized for the treatment of a SFRS8, CD83, SLAMF1, CD86, HRH1, IL2, TLR7, TLR8 and/or TLR10 gene associated disorder such as SCH and/or BPD. Such treatment can be performed, for example, in the form of gene replacement therapy. Specifically, one or more copies of a normal SFRS8, CD83, SLAMF1, CD86, HRH1, IL2, TLR7, TLR8 and/or TLR10 gene or a portion of said gene that directs the production of a gene product exhibiting normal SFRS8, CD83, SLAMF1, CD86, HRH1, IL2, TLR7, TLR8 and/or TLR10 gene function, may be inserted into the appropriate cells within a patient, using vectors that include, but are not limited to adenovirus, adeno-associated virus, and retrovirus vectors, in addition to other particles that introduce DNA into cells, such as liposomes.

Gene replacement therapy techniques should be capable delivering the SFRS8, CD83, SLAMF1, CD86, HRH1, IL2, TLR7, TLR8 and/or TLR10 gene sequences to cells expressing the corresponding gene within patients. Thus, in one embodiment, techniques that are well known to those of skill in the art (see, e.g., PCT Publication No. WO89/10134, published Apr. 25, 1988) can be used to enable the SFRS8, CD83, SLAMF1, CD86, HRH1, IL2, TLR7, TLR8 and/or TLR10 gene sequences to be uptaken by the cells. Viral vectors may advantageously be used for the purpose. Also included are methods using liposomes either in vivo ex vivo or in vitro. Wherein the SFRS8, CD83, SLAMF1, CD86, HRH1, IL2, TLR7, TLR8 and/or TLR10 gene sense or antisense DNA is delivered to the cytoplasm and nucleus of target cells. Liposomes can deliver the SFRS8, CD83, SLAMF1, CD86, HRH1, IL2, TLR7, TLR8 and TLR10 gene sense or nonsense RNA to humans and the lungs or skin through intrathecal delivery either as part of a viral vector or as DNA conjugated with nuclear localizing proteins or other proteins that increase take up into the cell nucleus.

In another embodiment, techniques for delivery involve direct administration of such SFRS8, CD83, SLAMF1, CD86, HRH1, IL2, TLR7, TLR8 and/or TLR10 gene sequences to the site of the cells in which the SFRS8, CD83, SLAMF1, CD86, HRH1, IL2, TLR7, TLR8 and/or TLR10 gene sequences are to be expressed, in particular the lungs and skin. Additional methods that may be utilized to increase the overall level of the SFRS8, CD83, SLAMF1, CD86, HRH1, IL2, TLR7, TLR8 and/or TLR10 gene expression and/or the SFRS8, CD83, SLAMF1, CD86, HRH1, IL2, TLR7, TLR8 and/or TLR10 gene product activity include the introduction of appropriate SFRS8, CD83, SLAMF1, CD86, HRH1, IL2, TLR7, TLR8 and/or TLR10 geneexpressing cells, preferably autologous cells, into a patient at positions and in numbers that are sufficient to ameliorate the symptoms of a SFRS8, CD83, SLAMF1, CD86, HRH1, IL2, TLR7, TLR8 and/or TLR10 gene associated disorder, such as SCH and/or BPD. Such cells may be either recombinant or non-recombinant.

Among the cells that can be administered to increase the overall level of SFRS8, CD83, SLAMF1, CD86, HRH1, IL2, TLR7, TLR8 and/or TLR10 gene expression in a patient are normal cells, preferably brain cells and also choroid plexus cells within the CNS which are accessible through intrathecal injections. Alternatively, cells, preferably autologous cells, can be engineered to express SFRS8, CD83, SLAMF1, CD86, HRH1, IL2, TLR7, TLR8 and/or TLR10 gene sequences, and may then be introduced into a patient in positions appropriate for the amelioration of the symptoms of a SFRS8, CD83, SLAMF1, CD86, HRH1, IL2, TLR7, TLR8 and/or TLR10 gene asoociated disorder. Alternately, cells that express an unimpaired SFRS8, CD83, SLAMF1, CD86, HRH1, IL2, TLR7, TLR8 and/or TLR10 gene and that are from a MHC matched individual can be utilized, and may include, for example, brain cells. The expression of the SFRS8, CD83, SLAMF1, CD86, HRH1, IL2, TLR7, TLR8 and/or TLR10 gene derived sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above that are capable of modulating the SFRS8, CD83, SLAMF1, CD86, HRH1, IL2, TLR7, TLR8 and/or TLR10 gene product activity can be administered using standard techniques that are well known to those of skill in the art.

### Modulation Co-Stimulatory Signal in T Cell Activation

One of non-limited examples of disorders where therapeutic compounds, such as described herein, may be used for treatment is a disorder involving initiation of co-stimulatory signal in T cell activation described below.

Induction of an immune response requires that T cells receive 2 sets of signals from antigen-presenting cells. The first signal is delivered through the T-cell receptor complex, while the second, or co-stimulatory, signal is provided by the B-cell activation antigens B7-1, or CD80, and B7-2, or CD86, by interaction with the T-cell surface molecules, CD28 and CTLA4.

The B7 molecules (CD80 and CD86) are homodimeric members of the immunoglobulin superfamily that are found exclusively on the surface of cells that can stimulate T-cell proliferation. Their role in co-stimulation has been demonstrated by transfecting fibroblasts that express a T-cell ligand with genes encoding B7 molecules and showing that the fibroblasts could then stimulate the clonal expansion of naïve T cells. The receptor for B7 molecules on the T cell is CD28, yet another member of the immunoglobulin superfamily. Ligation of CD28 by B7 molecules or by anti-CD28 antibodies co-stimulates the clonal expansion of naïve T cells, whereas anti-B7 antibodies, which inhibit the binding of B7 molecules to CD28, inhibit T cell responses. Although other molecules have been reported to co-stimulate naïve T cells, so far only the B7 molecules have been shown definitively to provide co-stimulatory signals for naïve T cells in normal immune responses.

Once a naïve T cell is activated, however, it expresses a number of proteins that contribute to sustaining or modifying the co-stimulatory signal that drives clonal expansion and differentiation. One such protein is CD40 ligand, so-called because it binds to CD40 on antigen-presenting cells. Binding of CD40 ligand by CD40 transmits activating signals to the T cell and also activates the antigen-presenting cell to express B7 molecules, thus stimulating further T-cell proliferation. CD40 and CD40 ligand belong to the TNF family of receptors and ligand and have a central role in the effector function of fully differentiated T cells. Their earlier role in sustaining the development of a T-cell response is demonstrated by mice lacking CD40 ligand; when these mice are immunized, the clonal expansion of responding T cells is curtailed at an early stage. Another pair of TNF family molecules that appear to contribute to co-stimulation of T cells are the T-cell molecule 4-1 BB (CD137) and its ligand 4-1 BBI, which is expressed on activated dendritic cells, macrophages, and B cells. As with CD40L and CD40, the effects of this receptor-ligand interaction are bi-directional, with both T cell and the antigen-presenting cell receiving activating signals; this process is sometimes referred to as the T-cell/antigen-presenting cell dialogue.

CD28-related proteins are also induced on activated T cells and serve to modify the co-stimulatory signal as the T-cell response develops. One is CTLA-4 (CD152), an additional receptor for B7 molecules. CTLA-4 closely resembles CD28 in sequence, and the two proteins are encoded by closely linked genes. However, CTLA-4 binds B7 molecules about 20 times more avidly than does CD28 and delivers an inhibitory signal to the activated T cell. This makes the activated progeny of a naïve T cell less sensitive to stimulation by the antigen-presenting cell and limits the amount of an autocrine T-cell growth factor, interleukin-2 (IL-2), that is produced. Thus, binding of CTLA-4 to B7 molecules is essential for limiting the proliferative response of activated T cells to antigen and B7. This was confirmed by producing mice with disrupted CTLA-4 gene; such mice develop a fatal disorder characterized by massive lymphocyte proliferation.

A third CD28-related protein is induced on activated T cells and can enhance T-cell responses; this inducible co-stimulator, or ICOS, binds a ligand known as LICOS, the ligand of ICOS, which is distinct from B7.1 and B7.2. LICOS is produced on activated dendritic cells, monocytes and B cells, but its contribution to immune responses has not yet been clearly defined. Although it resembles CD28 in driving T-cell growth, it differs from CD28 in not inducing IL-2; instead, it induces IL-10.

Thus, antigen-presenting cells engage in a co-stimulatory dialogue with T cells that recognize the antigens they display. This dialogue involves the delivery and receipt of signals through a number of different molecules, but appears to be initiated through the binding of B7 molecules to CD28 on a naïve T cell. Antigen-presenting cells are activated to express B7 molecules on detecting the presence of infection through receptors of the innate immune system. The requirement for the simultaneous delivery of antigen-specific and co-stimulatory signals by one cell in the activation of naïve T cells means that only such activated antigen-presenting cells, principally the dendritic cells that migrate into lymphoid tissue after being activated by binding and ingesting pathogens, can initiate T-cell responses. This is important, because not all potentially self-reactive T cells are deleted in the thymus; peptides derived from proteins made only in specialized cells in peripheral tissues might not be encountered during negative selection of thymocytes. Self-tolerance could be broken if naïve autoreactive T cells could recognize self antigens on tissue cells and then be co-stimulated by an antigen-presenting cells, either locally or at a distant site. Thus, the requirement that the same cell presents both the specific antigen and the co-stimulatory signal is important in preventing destructive immune responses to self tissues. Indeed, antigen binding to the T-cell receptor in the absence of co-stimulation not only fails to activate the cell, it instead leads to a state called anergy, in which the T cell becomes refractory to activation by specific antigen even when the antigen is subsequently presented to it by a professional antigen-presenting cell.

B7-2 mRNA is constitutively expressed in unstimulated B cells. The predicted protein is a type I membrane protein of the immunoglobin superfamily.

A soluble form of CD86 in human serum can be generated either by shedding of the membrane form or through alternative splicing. RT-PCR analysis revealed the expression of 2 transcripts in nonstimulated monocytes but only the full-length transmembrane form in activated monocytes. The smallest transcript, 828 bp, which the authors termed CD86delta™, has a deletion from nucleotide 686 to nucleotide 829 (i.e., exon 6) and encodes a 275-amino acid protein. SDS-PAGE and Western blot analysis detected expression of CD86 and CD86delta™ in COS cells as 65- and 48-kD proteins, respectively. FACS analysis detected only CD86 transfected cells and ELISA analysis detected only CD86delta™ in cell-free supernatants. Binding analysis demonstrated that CD86delta™ binds to CD28- or CTLA4-expressing cells. Functional analysis indicated that CD86delta™ enhances proliferation and cytokine production by both naive and memory T cells.

Resting eosinophils express neither MHC class II proteins nor costimulatory B7 molecules and fail to induce proliferation of T cells to antigens. It is known that IL3 induces expression of HLA-DR and B7.2 on eosinophils, but, unlike IL5 and GMCSF, it does not induce expression of B7.1. IL3-treated eosinophils supported modest T-cell proliferation in response to superantigen toxic shock syndrome-1 antigen, as well as proliferation of HLA-DR-restricted T-cell clones to tetanus toxoid (TT) and influenza virus antigenic peptides. The response was blocked by anti-B7.2 monoclonal antibody. IL3-treated eosinophils were unable to present native TT antigen to either resting or TT-specific cloned T cells. Parallel experiments established that IL5 and GMCSF induce T-cell proliferation to peptides but not to native TT antigen. It was suggested that eosinophils activated by IL3 may contribute to T-cell activation in allergic and parasitic diseases by presenting superantigens and peptides to T cells (Celestin et al., J. Immun. 167: 6097-6104, 2001).

The B7-2 gene is composed of 8 exons and spans more than 22 kb. The authors found that alternatively spliced cDNAs result from the use of either exon 1 or 2. Exon 3 corresponds to the signal peptide, exon 4 to an IgV-like domain, exon 5 to an IgC-like domain and exon 6 corresponds to the transmembrane region and part of the cytoplasmic tail. Exons 7 and 8 encode the remainder of the tail.

The B7-1 gene has 6 exons that span approximately 32 kb of genomic DNA. Exon 1 is not translated, and exon 2 contains the initiation ATG codon and encodes a predicted signal peptide. Exons 3 and 4 correspond to 21 g-like domains, whereas exons 5 and 6, respectively, encode the transmembrane portion and the cytoplasmic tail. This close relationship between exons and functional domains is a characteristic feature of genes of the Ig superfamily.

It was demonstrated that the CD86 and CD80 genes are linked on human chromosome 3 and mouse chromosome 16 (Reeves et al., Mammalian Genome 8: 581-582, 1997).

Thus, it is an aspect of the invention to use a compound capable of decreasing or modulating the co-stimulatory signal in T-cell activation for the preparation of a medicament for the treatment of allergy related diseases in a subject being diagnosed as having a predisposition to an immune related disease selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema.

In one embodiment the compound may be selected from corticosteroids, antihistamins, or brochodilatators. In another embodiment the compound may be a soluble variant llel 79Val B7-2 protein or an antibody directed against wild-type B7-2 protein such as described above.

It is understood that the immune related disease as above is determined according to a method of the invention.

### 9. Immunotherapy

The subjects carrying the mutations as defined in the present invention may also be treated using immunotherapy. The principles behind immunotherapy are described in short below.

The concept of vaccination is based on two fundamental characteristics of the immune system: specificity and memory. Vaccination primes the recipient's immune system and, upon repeated exposure to the same proteins, the immune system is in a position to respond more vigorously to the challenge of, for example, a microbial infection. Vaccines are mixtures of proteins for use in generating such protective immune responses in the recipient. The protection comprises only components present in the vaccine.

### Specific Allergy Vaccination

The aim of specific allergy vaccination is the generation of a protective immune response in the recipient, which will reduce or abolish allergic reactions. The vaccination strategy is based on the two features of the immune system referred to in the introduction: specificity and memory. However, patients with allergies already experience an adverse immunological reaction to the proteins relevant to vaccination. For this reason, a different protocol is used in specific allergy vaccination. Instead of administering one or a few high-dose injections, several low-dose injections are given. The protocol may be divided into two parts, an updosing phase and a maintenance phase. In the updosing phase, doses of increasing size are given under careful supervision. A higher, well tolerated dose is selected for the maintenance phase and given over a prolonged period, to attain an effective accumulated dose. Specific allergy vaccination is the only current treatment that permanently modifies the basic pathophysiological mechanisms of allergic patients' immune responses.

### Long-Term Effects of Specific Allergy Vaccination

The long-term clinical effect after termination of two to three years of specific allergy vaccination has been shown for grass pollen, tree pollen as well as animal hair and dander. In a study with patients allergic to grass pollen, it was shown that patients suffering from rhinoconjunctivitis with or without mild-to-moderate seasonal asthma had persistently and significantly fewer symptoms during seasonal exposure five years after termination of specific allergy vaccination when standardised allergen vaccine was used. A similar study with patients allergic to birch pollen showed an effect on asthma and hay-fever symptoms as well as nasal sensitivity after two years of specific allergy vaccination. This study confirms that the clinical effect persists for a period of at least 6 years after termination of treatment. The patients had significantly fewer symptoms compared with the level at the termination of treatment, despite the fact that exposure during the follow-up season was 75 times higher than in the season of inclusion. Another interesting result from this study was that none of the patients who initially suffered only from hay-fever developed asthma during the study period [Jacobsen L, Nüchel Petersen B, Wihl J Å, Løwenstein H, Ipsen H: Immunotherapy with partially purified and standardised tree pollen extracts. IV. Results from long-term (6-year) follow-up. Allergy 52:914-920, 1997].

Patients allergic to cats who have mild to moderate asthma have been shown not only to reduce their reactivity to cat allergen but also to reduce non-specific hyperreactivity and hypersensitivity estimated using a histamine challenge test. In the follow-up study five years after termination of specific allergy vaccination, the effect was persistent with regard to exposure to cats as well as non-specific hyperreactivity [Hedlin G, Heilborn H, Lilja G, Norrlind K, Pegelow K O, Schou C, Løwenstein H. Long-term follow-up of patients treated with a three-year course of cat or dog immunotherapy. J Allergy Clin Immunol 96:879-885, 1995].

### Anti-Inflammatory Effect of Specific Allergy Vaccination

In asthmatic people allergic to birch pollen, specific allergy vaccination has been found to cause a significant suppression of the increase in eosinophilic cationic protein (ECP) during the season. Furthermore, patients treated with specific allergy vaccination had significantly improved lung function (FEV1, PEF, and PC20) during seasonal exposure when compared to patients treated with placebo [Hakansson L, Heinrich C, Rak S, Venge P: Priming of eosinophil adhesion in patients with birch pollen allergy during pollen season: effect of immunotherapy. J Allergy Clin Immunol 99:551-62, 1997].

It has been demonstrated that late-phase skin reaction after intracutaenous challenge with allergens is significantly reduced in actively treated patients compared with placebo. During the four-year period of specific allergy vaccination, a persistent reduction in late-phase skin reaction was observed, while the early skin reaction returned to initial values despite the clinical improvements.

The hypothesis that specific allergy vaccination has an anti-inflammatory effect has been brought forward and it is proposed that a switch in T-helper cells from TH2 to TH1, followed by an increase in interferon gamma production, might be a part of the basic effector mechanism of specific allergy vaccination.

### Preventive Allergy Treatment

Studies on the long-term effect of specific allergy vaccination have indicated that the treatment may prevent exacerbation from hay-fever to asthma. A study has shown that fewer patients developed non-specific bronchial hypersensitivity if they were treated by specific allergy vaccination.

### 10. Drug Discovery

A cell line based on cells isolated from a subject carrying a polymorphism according to the invention may also be cultured and used for the screening purposes.

The vector may comprise part(s) of the nucleotide sequence of SEQ ID NOs: 1-9, or SEQ ID NOs: 10-18, said sequence comprising a polymorphism associated with an immune-related disease. Using this vector more precisely mimics the expression in vivo due to the presence of introns and possibly the native promoter of the genes.

According to some embodiments the vector may comprise a constitutive promoter. According to other embodiments the vector may comprise a promoter sequence comprising a regulatable promoter such as a viral promoter sequence.

The vector may be transferred into a host cell which can be used for screening purposes in drug discovery. The host cells may be selected from a bacterial cell, a yeast cell, a mammalian cell line, more preferably a human cell line. More preferably, the host cell is a human immortalised cell line such as human melanocyte.

Screening of compounds for a functionality related to immune response can be carried out by exposing a cell as described above to a drug candidate and measuring a response related to the co-stimulatory signal and induction of immune response.

The response may for example be selected from the group comprising: T-cell activation, proliferation of T-cells, a change in the relative amount of CD45 splice isoforms or cytokines, preferably, the cytokines are selected from the group comprising IL4, IL5, IL10, and IL13, activation of JAK-STAT signalling pathways, or binding of B7-2 to CD28 and/or to CTLA4.

Screening methods for compounds with are capable of modulating the SFRS8, CD83, SLAMF1, CD86, HRH1, IL2, TLR7, TLR8 and/or TLR10 protein-protein interactions are within the scope of the invention.

For the purpose of below discussion molecules that produced in the cells due to activity of the SFRS8, CD83, SLAMF1, CD86, HRH1, IL2, TLR7, TLR8 and/or TLR10 genes, such as transcriptional and translational products of the genes, are termed herein “gene products”, if not specified otherwise.

Any method suitable for detecting protein-protein interactions may be employed for identifying the SFRS8, CD83, SLAMF1, CD86, HRH1, IL2, TLR7, TLR8 and/or TLR10 protein-protein interactions.

Among the traditional methods that may be employed are co-immunoprecipitation, cross-linking and co-purification through gradients or chromatographic columns. Utilizing procedures such as these allows for the identification of proteins, including intracellular proteins, which interact with SFRS8, CD83, SLAMF1, CD86, HRH1, IL2, TLR7, TLR8 and/or TLR10 proteins. Once isolated, such a protein can be identified and can be used in conjunction with standard techniques, to identify proteins it interacts with. For example, at least a portion of the amino acid sequence of a protein that interacts with SFRS8, CD83, SLAMF1, CD86, HRH1, IL2, TLR7, TLR8 and/or TLR10 protein can be ascertained using techniques well known to those of skill in the art, such as via the Edman degradation technique (see, e.g., Creighton, 1983, “Proteins: Structures and Molecular Principles,” W.H. Freeman & Co., N.Y., pp. 34-49). The amino acid sequence obtained may be used as a guide for the generation of oligonucleotide mixtures that can be used to screen for gene sequences encoding such proteins. Screening made be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of genes that encode a protein which interacts with SFRS8, CD83, SLAMF1, CD86, HRH1, IL2, TLR7, TLR8 and/or TLR10 protein. These methods include, for example, probing expression libraries with labelled SFRS8, CD83, SLAMF1, CD86, HRH1, IL2, TLR7, TLR8 and/or TLR10 polypeptides, using SFRS8, CD83, SLAMF1, CD86, HRH1, IL2, TLR7, TLR8 and/or TLR10 proteins in a manner similar to the well known technique of antibody probing of lambda.gtll and lambda.gt10 libraries.

One method that detects protein interactions in vivo, the two-hybrid system, is described in detail for illustration only and not by way of limitation. One version of this system has been described (Chien, et al., 1991, Proc. Natl. Acad. Sci. USA, 88, 9578-9582) and is commercially available from Clontech (Palo Alto, Calif.).

Briefly, utilizing such a system, plasmids are constructed that encode two hybrid proteins: one consists of the DNA-binding domain of a transcription activator protein fused to the SFRS8, CD83, SLAMF1, CD86, HRH1, IL2, TLR7, TLR8 and/or TLR10 gene peptide product and the other consists of the transcription activator protein's activation domain fused to an unknown protein that is encoded by a cDNA that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, SFRS8, CD83, SLAMF1, CD86, HRH1, IL2, TLR7, TLR8 and/or TLR10 gene derived peptide products may be used as the bait gene product. Total genomic or cDNA sequences are fused to the DNA encoding an activation domain. This library and a plasmid encoding a hybrid of a bait SFRS8, CD83, SLAMF1, CD86, HRH1, IL2, TLR7, TLR8 and/or TLR10 protein, or a fragment thereof, fused to the DNA-binding domain are co-transformed into a yeast reporter strain, and the resulting transformants are screened for those that express the reporter gene. For example, and not by way of limitation, a bait SFRS8, CD83, SLAMF1, CD86, HRH1, IL2, TLR7, TLR8 and/or TLR10 gene sequence, such as the open reading frame of the SFRS8, CD83, SLAMF1, CD86, HRH1, IL2, TLR7, TLR8 and/or TLR10 gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait SFRS8, CD83, SLAMF1, CD86, HRH1, IL2, TLR7, TLR8 and/or TLR10 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait SFRS8, CD83, SLAMF1, CD86, HRH1, IL2, TLR7, TLR8 and/or TLR10 gene sequence-GAL4 fusion plasmid into a yeast strain that contains a lacZ gene driven by a promoter that contains GAL4 activation sequence. A cDNA encoded protein, fused to GAL4 transcriptional activation domain, that interacts with bait SFRS8, CD83, SLAMF1, CD86, HRH1, IL2, TLR7, TLR8 and/or TLR10 gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait SFRS8, CD83, SLAMF1, CD86, HRH1, IL2, TLR7, TLR8 and/or TLR10 protein-interacting protein using techniques routinely practiced in the art.

The invention also related to screening assays for compounds that interfere with the SFRS8, CD83, SLAMF1, CD86, HRH1, IL2, TLR7, TLR8 and/or TLR10 gene products macromolecule interaction.

The SFRS8, CD83, SLAMF1, CD86, HRH1, IL2, TLR7, TLR8 and/or TLR10 gene products of the invention may, in vivo, interact with one or more macromolecules, including intracellular macromolecules, such as proteins. Such macromolecules may include, but are not limited to, nucleic acid molecules and those proteins identified via methods such as those described above. For purposes of this discussion, the macromolecules are referred to herein as “binding partners”. Compounds that are able to disrupt the SFRS8, CD83, SLAMF1, CD86, HRH1, IL2, TLR7, TLR8 and TLR10 gene products binding in this way may be useful in regulating the activity of products of the SFRS8, CD83, SLAMF1, CD86, HRH1, IL2, TLR7, TLR8 and TLR10 genes, especially variant SFRS8, CD83, SLAMF1, CD86, HRH1, IL2, TLR7, TLR8 and TLR10 proteins and thereof derived peptide products. Such compounds may include, but are not limited to molecules such as peptides, and the like, which would be capable of gaining access to a SFRS8, CD83, SLAMF1, CD86, HRH1, IL2, TLR7, TLR8 and/or TLR10 gene product.

The basic principle of the assay systems used to identify compounds that interfere with the interaction between SFRS8, CD83, SLAMF1, CD86, HRH1, IL2, TLR7, TLR8 and TLR10 gene products and their binding partner or partners involves preparing a reaction mixture containing the SFRS8, CD83, SLAMF1, CD86, HRH1, IL2, TLR7, TLR8 and/or TLR10 gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of SFRS8, CD83, SLAMF1, CD86, HRH1, IL2, TLR7, TLR8 and/or TLR10 gene product and its binding partner. Control reaction mixtures are incubated without the test compound or with a placebo. The formation of any complexes between the SFRS8, CD83, SLAMF1, CD86, HRH1, IL2, TLR7, TLR8 and/or TLR10 gene product and the binding partner is then detected. The formation of a complex in the control reaction, but not in the reaction mixture containing the test compound, indicates that the compound interferes with the interaction of the SFRS8, CD83, SLAMF1, CD86, HRH1, IL2, TLR7, TLR8 and/or TLR10 gene product and the interactive binding partner. Additionally, complex formation within reaction mixtures containing the test compound and for example normal (wild type) SFRS8, CD83, SLAMF1, CD86, HRH1, IL2, TLR7, TLR8 and/or TLR10 protein may also be compared to complex formation within reaction mixtures containing the test compound and a variant SFRS8, CD83, SLAMF1, CD86, HRH1, IL2, TLR7, TLR8 and/or TLR10 protein. This comparison may be important in those cases wherein it is desirable to identify compounds that disrupt interactions of mutant but not wild type SFRS8, CD83, SLAMF1, CD86, HRH1, IL2, TLR7, TLR8 and/or TLR10 protein.

The assay for compounds that interfere with the interaction of SFRS8, CD83, SLAMF1, CD86, HRH1, IL2, TLR7, TLR8 and/or TLR10 gene products and their binding partners can be conducted in a heterogeneous or homogeneous format. Heterogeneous assays involve anchoring either the SFRS8, CD83, SLAMF1, CD86, HRH1, IL2, TLR7, TLR8 and/or TLR10 gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the SFRS8, CD83, SLAMF1, CD86, HRH1, IL2, TLR7, TLR8 and/or TLR10 gene products and the binding partners, e.g., by competition, can be identified by conducting the reaction in the presence of the test substance; i.e., by adding the test substance to the reaction mixture prior to or simultaneously with the SFRS8, CD83, SLAMF1, CD86, HRH1, IL2, TLR7, TLR8 and/or TLR10 gene protein and interactive intracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the SFRS8, CD83, SLAMF1, CD86, HRH1, IL2, TLR7, TLR8 and/or TLR10 gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the SFRS8, CD83, SLAMF1, CD86, HRH1, IL2, TLR7, TLR8 and/or TLR10 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labelled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of a SFRS8, CD83, SLAMF1, CD86, HRH1, IL2, TLR7, TLR8 and/or TLR10 gene product and the interactive binding partner is prepared in which either the SFRS8, CD83, SLAMF1, CD86, HRH1, IL2, TLR7, TLR8 and/or TLR10 gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt the SFRS8, CD83, SLAMF1, CD86, HRH1, IL2, TLR7, TLR8 and TLR10 gene product/binding partner interaction can be identified.

In another embodiment, the SFRS8, CD83, SLAMF1, CD86, HRH1, IL2, TLR7, TLR8 and/or TLR10 gene product can be prepared for immobilization using recombinant DNA techniques. For example, the SFRS8, CD83, SLAMF1, CD86, HRH1, IL2, TLR7, TLR8 and/or TLR10 gene coding region can be fused to the glutathioneS-transferase (GST) gene using a fusion vector, such as pGEX-5×−1, in such a manner that its binding activity is maintained in the resulting fusion protein. The interactive binding partner can be purified and used to raise an antibody, using methods routinely practiced in the art. The antibody can then be labeled with a radioactive isotope such as .sup.125 I, for example, by methods routinely practiced in the art. In a heterogeneous assay, e.g., the GST-SFRS8, CD83, SLAMF1, CD86, HRH1, IL2, TLR7, TLR8 and/or TLR10 fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the SFRS8, CD83, SLAMF1, CD86, HRH1, IL2, TLR7, TLR8 and/or TLR10 gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-SFRS8, CD83, SLAMF1, CD86, HRH1, IL2, TLR7, TLR8 and/or TLR10 fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the SFRS8, CD83, SLAMF1, CD86, HRH1, IL2, TLR7, TLR8 and/or TLR10 gene product/binding partner interaction can be detected by adding the labelled antibody and measuring the radioactivity associated with the beads.

In still another embodiment of the invention, these same techniques can be employed using peptide fragments that correspond to the binding domains of SFRS8, CD83, SLAMF1, CD86, HRH1, IL2, TLR7, TLR8 and/or TLR10 proteins and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labelled peptide comprising the binding domain may remain associated with the solid material, which can be isolated and identified by amino acid sequencing. Also, once the gene coding for the segments can be engineered to express peptide fragments of the protein, which can then be tested for binding activity and purified or synthesized.

For example, and not by way of limitation, a SFRS8, CD83, SLAMF1, CD86, HRH1, IL2, TLR7, TLR8 and/or TLR10 gene product can be anchored to a solid material as described above by making a GST-SFRS8, CD83, SLAMF1, CD86, HRH1, IL2, TLR7, TLR8 and/or TLR10 fusion protein and allowing it to bind to glutathione agarose beads. The interactive binding partner obtained can be labeled with a radioactive isotope, such as .sup.35 S, and cleaved with a proteolytic enzyme such as trypsin. Cleavage products can then be added to the anchored GST-SFRS8, CD83, SLAMF1, CD86, HRH1, IL2, TLR7, TLR8 and/or TLR10 fusion protein and allowed to bind. After washing away unbound peptides, labelled bound material, representing the binding partner binding domain, can be eluted, purified, and analyzed for amino acid sequence by well-known methods. Peptides so identified can be produced synthetically or fused to appropriate facilitative proteins using recombinant DNA technology.

The invention also provides assays for identification of compounds that ameliorate the SFRS8, CD83, SLAMF1, CD86, HRH1, IL2, TLR7, TLR8 and TLR10 gene associated disorders, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

Compounds, including but not limited to binding compounds identified via assay techniques such as those described above can be tested for the ability to ameliorate symptoms of a SFRS8, CD83, SLAMF1, CD86, HRH1, IL2, TLR7, TLR8 and/or TLR10 gene associated disorder including immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

It should be noted that the assays described herein can identify compounds that affect the SFRS8, CD83, SLAMF1, CD86, HRH1, IL2, TLR7, TLR8 and/or TLR10 gene activity by either affecting SFRS8, CD83, SLAMF1, CD86, HRH1, IL2, TLR7, TLR8 and TLR10 gene expression or by affecting the level of SFRS8, CD83, SLAMF1, CD86, HRH1, IL2, TLR7, TLR8 and TLR10 gene product activity. For example, compounds may be identified that are involved in another step in the pathway in which the SFRS8, CD83, SLAMF1, CD86, HRH1, IL2, TLR7, TLR8 and/or TLR10 gene and/or the SFRS8, CD83, SLAMF1, CD86, HRH1, IL2, TLR7, TLR8 and/or TLR10 gene product is involved and, by affecting this same pathway may modulate the effect of the SFRS8, CD83, SLAMF1, CD86, HRH1, IL2, TLR7, TLR8 and/or TLR10 gene on the development of immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema. Such compounds can be used as part of a therapeutic method for the treatment of the disorder.

Described below are cell-based and animal model-based assays for the identification of compounds exhibiting such an ability to ameliorate symptoms of the SFRS8, CD83, SLAMF1, CD86, HRH1, IL2, TLR7, TLR8 and TLR10 gene activity associated with immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema.

First, cell-based systems can be used to identify compounds that may act to ameliorate symptoms of a SFRS8, CD83, SLAMF1, CD86, HRH1, IL2, TLR7, TLR8 and/or TLR10 gene associated disorder, such immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema. Such cell systems can include, for example, recombinant or non-recombinant cell, such as cell lines, that express the SFRS8, CD83, SLAMF1, CD86, HRH1, IL2, TLR7, TLR8 and/or TLR10 gene.

In utilizing such cell systems, cells that express the SFRS8, CD83, SLAMF1, CD86, HRH1, IL2, TLR7, TLR8 and/or TLR10 gene may be exposed to a compound suspected of exhibiting an ability to ameliorate symptoms of a SFRS8, CD83, SLAMF1, CD86, HRH1, IL2, TLR7, TLR8 and/or TLR10 gene disorder, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, at a sufficient concentration and for a sufficient time to elicit such an amelioration of such symptoms in the exposed cells. After exposure, the cells can be assayed to measure alterations in the expression of the SFRS8, CD83, SLAMF1, CD86, HRH1, IL2, TLR7, TLR8 and/or TLR10 gene, e.g., by assaying cell lysates for the presence of SFRS8, CD83, SLAMF1, CD86, HRH1, IL2, TLR7, TLR8 and/or TLR10 gene transcripts (e.g., by Northern analysis) or for the SFRS8, CD83, SLAMF1, CD86, HRH1, IL2, TLR7, TLR8 and TLR10 gene translation products expressed by the cell. Compounds that modulate expression of the SFRS8, CD83, SLAMF1, CD86, HRH1, IL2, TLR7, TLR8 and/or TLR10 gene are considered to be good candidates as therapeutics.

Alternatively, the cells are examined to determine whether one or more cellular phenotypes associated with a SFRS8, CD83, SLAMF1, CD86, HRH1, IL2, TLR7, TLR8 and/or TLR10 gene disorder, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, has been altered to resemble a more normal or unimpaired, unaffected phenotype, or a phenotype more likely to produce a lower incidence or severity of disorder symptoms.

In addition, animal-based systems or models for a SFRS8, CD83, SLAMF1, CD86, HRH1, IL2, TLR7, TLR8 and/or TLR10 gene associated disorder, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, which may include, for example mice, may be used to identify compounds capable of ameliorating symptoms of the disorder. Such animal models may be used as test substrates for the identification of drugs, pharmaceuticals, therapies and interventions that may be effective in treating such disorders. For example, animal models may be exposed to a compound suspected of exhibiting an ability to ameliorate symptoms, at a sufficient concentration and for a sufficient time to elicit such an amelioration of symptoms of a SFRS8, CD83, SLAMF1, CD86, HRH1, IL2, TLR7, TLR8 and/or TLR10 gene associated disorder, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, in the exposed animals. The response of the animals to the exposure may be monitored by assessing the reversal of such symptoms.

With regard to intervention, any treatments that reverse any aspect of symptoms of a SFRS8, CD83, SLAMF1, CD86, HRH1, IL2, TLR7, TLR8 and/or TLR10 gene associated disorder, such as immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, should be considered as candidates for human therapeutic intervention in such a disorder. In particular, the invention concerns candidate compounds capable of
i) modulating expression of a gene selected from the genes of the invention, said compound being selected from an isolated antisense nucleotide sequence or an nucleotide sequence complementary to the regulatory region of said gene, said nucleotide sequence being capable of forming triple helix structures that prevent transcription of said gene, and/or
ii) modulating activity of a transcriptional product of a gene selected from the genes of the invention, said transcriptional product being (1) a nucleotide sequence selected from SEQ ID NOs: 1-9, (2) a sequence having at least 90% sequence identity with SEQ ID NOs: 1-9, or a fragment thereof, and/or (3) a sequence complementary to one of these sequences or a fragment thereof,
   wherein said candidate compound is preferably selected from an isolated antisense sequence or a ribozyme molecule, and/or
iii) modulating activity of translational products of the genes of the invention, said translational products being variant proteins discussed above,
   wherein said candidate compound is preferably selected from an antibody molecule against said translational product, or a molecule capable of interfering with biological activity of said translational product.

The term “modulating” is meant in the present context both inhibiting and stimulating

By inhibiting or modulating the expression of the SFRS8 gene or products thereof it is possible modulating the alternative splicing of the CD45 gene or modulating the effect of the various splice-isoforms of CD45.

Accordingly, in another embodiment the invention relates to a compound with is capable of directly or indirectly modulate the activity of a gene interacting with a gene of the invention. The examples of the genes, activity of which is dependent on the activity of the genes of the invention or is related to the activity of one or more genes of the invention is described above.

The invention further relates to a pharmaceutical composition comprising a compound of the invention.

### 11. Pharmaceutical Composition

Once the candidate compound(s) of the invention has been identified it is further within the scope of the invention to provide a pharmaceutical composition comprising one or more compound(s). In the present context the term pharmaceutical composition is used synonymously with the term medicament.

The invention is further related to a pharmaceutical composition capable of preventing the symptoms of a SFRS8, CD83, SLAMF1, CD86, HRH1, IL2, TLR7, TLR8 and/or TLR10 gene associated disorder, such as an immune-related disorder including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, said composition comprising an effective amount of one or more of the compounds described above. The parmaceutical composition may further comprise compounds, in particular drugs or members of classes or families of drugs, known to ameliorate or exacerbate the symptoms of immune-related disorders including asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, with the use of anti-inflammatory drugs, glucocorticoids, antihistamines, allergen-specific immuno preparates, sympatomimetics, anti-astma compounds, such as alpha1, alpha 2, beta1 and beta2 antagonists, leukotrien receptor antagonist, such as montelukast, parasympatolytics, such as ipratropium, theophyllin and theophyllamin, croglicat, nedocromil and methorexat. The medicament of the invention may also comprise an effective amount of one or more of the compounds as defined above in combination with pharmaceutically acceptable additives.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 5000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. In particular, when administering nasally smaller dosages are used than when administering by other routes. Administration may be performed once or may be followed by subsequent administrations. The dosage will also depend on the route of administration and will vary with the age and weight of the subject to be treated. A preferred dosage of multimeric forms would be in the interval 1 mg to 70 mg per 70 kg body weight.

For some indications a localised or substantially localised application is preferred.

For other indications, intranasal application is preferred.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to development of an immune related disease of the invention. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In another aspect the invention relates to a process of producing a pharmaceutical composition, comprising mixing an effective amount of one or more of the compounds of the invention, or a pharmaceutical composition according to the invention with one or more pharmaceutically acceptable additives or carriers, and administer an effective amount of at least one of said compound, or said pharmaceutical composition to a subject.

In yet a further aspect the invention relates to a method of treating an individual suffering from one or more of the diseases discussed above by administering the said individual a compound as described herein or a pharmaceutical composition comprising said compound.

### 12. Therapeutic and Diagnostic Methods

As already discussed above, information provided by the present invention is to be used for diagnostic and therapeutic purposes.

In one embodiment the invention relates to a method for determining a predisposition for an immune-related disease or condition in a subject comprising determining in a biological sample isolated from said subject one or more polymorphisms in the chromosome regions containing the SFRS8, SLAMF1, CD83, CD86, TLR7, TLR8, and/or TLR10 genes or in a translational or transcriptional product from said regions, or comprising determining two or more polymorphisms in the SFRS8, SLAMF1, CD86, TLR7, TLR8, TLR10, IL2, CD83, and/or HRH1 genes or in a translational or transcriptional product of said gene, preferably determining the presence of an SNP(s) discussed above.

In another embodiment the invention relates to a method for determining a predisposition for not having an immune-related disease in a subject comprising determining in a biological sample isolated from said subject the protective allele of a polymorphism in the SFRS8, SLAMF1, CD86, TLR7, TLR8, TLR10, IL2, CD83, and/or HRH1 gene which was associated with an immune related disease of the invention, preferably a protective allent of a SNP(s) discussed above.

In still another embodiment the invention relates to a method for determining a protection against an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema, in a subject comprising determining in a biological sample isolated from said subject a protective allele of an SNP(s) selected form the SNP(s).

Further, the invention relates to a method for prognosis of the likelihood of development of an immune related disease comprising determining a polymorphism of a gene selected from the SFRS8, SLAMF1, CD86, TLR7, TLR8, TLR10, IL2, CD83 and/or HRH1 genes, said polymorphism being preferably an SNP associated with an immune related disease of the inventionas selected from the SNPs discussed above.

A method for prognosis of the likelihood of development of an immune related disease comprising determining a polymorphism of a gene selected from the genes of the invention, wherein the polymorphism is an SNP selected from the SNPs discussed above, is also in the scope of the invention.

Other embodiments of the invention relate to methods for treatment of an immune related disease, such as asthma, bronchial hyperresponsiveness, rhinitis/hayfever, conjunctivitis/rhino conjuntivitis, atopic dermatitis/eczema, systemic anaphylaxis, contact dermatitis, urticaria, hypersensitivity reactions types I-IV, oral allergy syndrome, allergic gastrointestinal reactions, systemic reactions after insect stings, angio oedema, in a subject being diagnosed as having a predisposition according to the invention, comprising
1) administering to said subject a therapeutically effective amount of a gene therapy vector, said gene therapy vector comprising the protective allele of an SNP associated with the immune related disease (discussed above), and/or
2) administering to said subject a therapeutically effective amount of a candidate drug compound of the invention (discussed above) or a pharmaceutical composition comprising thereof.

The invention also relates to a method for predicting the likelihood of a subject to respond to a therapeutic treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said method comprising determining the genotype of said subject in the chromosome areas comprising the SFRS8, CD83, SLAMF1, CD86, HRH1, IL2, TLR7, TLR8 and/or TLR10 gene.

With the knowledge of the present invention it is possible to design pharmaceutical treatment of the diagnosed subjects more precisely, because pharmaceuticals can be designed and used to decrease the expression of the genes and thus decrease the effect of the gene polymorphism. Thus, a patient having an immune related disease described in the application may be more effectively and without undesirable side effects treated.

### EXAMPLES

In order to identify potential susceptibility variants in the SFRS8, SLAMF1, CD86, CD83, HRH1, IL2, TLR7, TLR8, and TLR10 genes, the genes were sequenced in a subset of patients with allergic disorders. The genomic sequences containing upstream promoter sequences, intronic sequences close to the exon/intron boundaries and coding sequences were analysed. The identified variants were analysed in two independent Danish samples comprising, respectively, 100 (Sample 1) and 143 (Sample 2) families with at least two siblings suffering from allergic disorders.

### Sample 1 (AIA)

Nuclear families were recruited through four paediatric and one adult outpatient allergy clinics in Aalborg, Viborg, Herning and Aarhus all in the western part of Denmark. A family was selected for the study if at least two full siblings had doctors diagnosed symptoms of atopy, i.e. asthma, rhinitis or atopic dermatitis, and reported effect of appropriate medication. Participation of both the biological parents was necessary to qualify the family for the project. The total of 424 individuals, 200 parents and 224 children, were all clinically examined and questionnaire tested by one doctor. Each person had blood drawn for DNA analysis and for serum measurements of total IgE and specific IgE, RAST, to 11 common allergens. Mean age among the offspring was 10.8 years and male/female sex ratio was 1.2 equal to random distribution (p=0.35). All participants and/or their parents gave informed consent.

| | Parents | Offspring |
|---|---|---|
| Total number | 200 | 224 |
| Male/female ratio | 1 | 1.2 |
| Mean age (years) | 41.1 | 10.8 |
| Asthma | 31 | 158 |
| Atopic dermatitis | 34 | 118 |
| Rhinitis | 60 | 130 |
| Total IgE (100 kU/l | 69 | 137 |
| RAST (1+ | 66 | 139 |

Clinical features of the 100 sib-pair families of Sample 1. The number of individuals with each phenotype is listed for both parents and offspring. RAST (1+ indicates specific allergy to at least one of the eleven allergens tested.

### Sample 2 (VB)

143 nuclear families including 246 parents and 246 affected siblings suffering from asthma and other atopic disorders were ascertained. All individuals were clinically examined and questionnaire tested by a medical doctor. Each person had blood drawn for DNA analysis and for serum measurements of specific IgE, RAST. Individuals with asthmatic symptoms were tested for bronchial hyperresponsiveness. All participants and/or their parents gave informed consent.

The table in FIGS. 1-22 reports the statistical analysis of the association between the presence of specific alleles and allergy phenotypes, showing p-values obtained by the transmission disequilibrium test (TDT). Results are shown from analysis of each sample separately and from the combined analysis of both samples. “Sibs” signifies that both affected siblings were included in the analysis, whereas “trios” signifies that only a single, randomly chosen, affected child from each family was included.

The analysis presents evidence that SFRS8, SLAMF1, CD86, CD83, HRH1, IL2, TLR7, TLR8, and TLR10 are susceptibility genes for allergy phenotypes (and possibly other immune related disorders). The susceptibility effect appears to be mediated through the gene variants containing one or more SNPs. The effect is observed when the risky allele of a particular SNP is expressed. Alternatively, or additionally, the observed susceptibility may be mediated by accumulative effect of the presence of multiple SNPs in one or different individual genes, when these SNPs represent individual specific haplotypes, which tend to be inherited together. Moreover, some of the haplotypes observed are in linkage disequilibrium.

### Description of the Gene Sequences of the Invention

In the following DNA sequences a coding sequence is indicated by capital letters and non-coding sequence by small cases.

SLAMF1 genomic sequence

SEQ ID NO: 1

ccacaaatggtggggttacaggcgtgccactgtgcccatccagattcctgaaaatttaacaattttatgagttggtacatgctgactc
gagcacacaccactgggaatagttgtgaggaggacagttgagtgctggggaaaggaaggaagaaaacagtgaggataaag
ttcacatatctcaccagcttttattacctgatccccatggggaggcccatcagagagtgcctatgacctgttacaatggactctaaaa
acacttccctactctttcaagtctccctgtgagcattggttacacttccagtatcccattcttatagtttaactcatgaaaaagggcggg
atcctccttctgccaatactagttccttctcctcaatgaaaagttagacacaaactccaaaataaaggcaactcccagaatacaac
acagccccaattaaattaaaatggcttttatccaaaagacaggtaataacaaatgctgacaaggatgtggagaaaaagtaccct
tgtacactgttgttgggaatataagttagtacaaccactgtggagaatggtttgaaggttcctcaaaaaactaaaaatagagctacc
atatgatccacaatctcactggtaggtatacacctaaaagaaaagaaatcagtatattgaagagatatctgtactcccatgtttatta
cagcactattcacaatagccaaggttggaagcgacctaagcgtctatcaccgatgagtggataaagaaaatgtggtacatatac
acaatgaagtactattcagctaaaaaagaattagatcctgtcattcacaatgacatggatggaattgaagatcattatgttacgtga
aataagccaggcacagacagacaaactttgcacgttctcacttgcttgtgagaggtaaaaattaaaacaattgaacttgtgggcat
agagagtagaaggatggttaccagaggctgaagggtagtgggggttggggaagaagtggggatggttaatgggtacaaaaa
aatagaaagaatgaataagaactagtatttgataatacaacagtgtgactatagtcaataataatttaattgcacatttaaaaataa
aaatataattgcactgtttgtaacacaaaggataaatatttgaggtgatggatatcccatttaccctgtgtgattattacatattgcatgc
ctctatcaacatatctcatataccccatacaaatatatgcataccccatacatatatatatacatacacacacacacacacacaca
cacacacacacacacacacacacatatatgtatatctactatgtacccacagacgttaaaaattagaggagaaaacacacaca
caacaaggagactgagctggaaggatggagctctgggatagatttgtcctacatccctgcctgggagggaatccacacacatg
caagaagacaaactaggagcatgggctactaaattataccacattgcactcatcggggtcacagggtttcttccaagtgacccgc
acatgcccttcccatctctgtgtgacagtggcacctgcaccagactgcatgttgaggtgtcatctgaaattatgaaataaaacagaa
gtaagaggtctattagctcatcaaaatgcagttatctaagttcagctgtgaactgccaaatttgaggagtgatccaatgaaacatctt
ttctttgcaatccaagaagacttaccggagagaactgctcagagaatctgcaacatccggttcctggagacagctaaggaaaga
agctggggcgcatgtttctgcccaaagccgggttttggccgaggtgactacacaccccctttcctggctcccataggctaagtgcct
ggcttcttgagaagcctgcttcttgagaacaaaaaagtgatttaaagcctcatgggagatgagcaatcctcaagacacaagcag
aaaaagtcccagtgatacaggaagcgGGTTCAGGAACCTGCTGGTTCCTGATACATAAATCAGACA
GCCTCTGCTGCATGACACGAAGCTTGCTTCTGCCTGGCATCTGTGAGCAGCTGCCAGG
CTCCGGCCAGGATCCCTTCCTTCTCCTCATTGGCTGATGGATCCCAAGGGGCTCCTCT
CCTTGACCTTCGTGCTGTTTCTCTCCCTGGCTTTTGGGGCAAGCTACGGAACAGgtgagtg
ttcatctgcctgatggtttgagtcccatgttagctgccaggaatcagcgtatcttcgtggatggagagaaggtgcagggctgggtatt
gtgtttggtcactcttccttagggactggctgtcagtttcaactgcctctttcaaagaggaaggaacattataagttcctgggcccttgg
gtttccaagactcagccccaccaaccccagtttccaaggaaatgaggggctctaagccaaaggctccagtcacttttctgaccag
tcttagggtgacaggccctggtagaagtcttgcttgagtggttggttttacatgggcatcttctggcaaagacccagcctagagaga
ctgagctggatggactgagctctgggagaagatttgccctacatccctgccctgggagggaatctgtgcacatgcaggctgacaa
accaggagcatgggtcaacagaaagcattggctagagtgggaagagagagtagaagtgaaaactccaggcttttggctgaga
accagcagtggccacagtgcggtcatactggtgtgtattttcttggaagagaaggtccaagaaagcaagagggaagaagttgg
gatttctgaaggctagggctggttacagtatgtgggaaatgcaaattgggaaccctcagagagtagctccagcaggaaggcca
gacaagagctacctttggatctggactctgttcctgtctttctgtctatcttcttcccaaggcaggctattgctttctgtttagaagtatcag
ggctatgagaaaaggtatttgagaaagaaaaagccaagcaagaagtggactttggactgcctgtgtgagtggggtgagaatct
ccttctgcttatttgtttagactgtgggaggtagcctggagtagaagaggtggcattacggacacggggggaaatcctgaggccca
gggtgttttaagcttggggttttcaagaccgcaaatccaatatggacttttccaggaaaagcaccgtgatatgccagggatgtggg
ggtgctgcacaatggatgtgtcttttaccagacagccagacgaacagggcttgctcagcccactttcttttggaatctgcagatccat
ggctcgtacttcccaaggtctaggggaggaagaactgagctcggggctcagaaaaccaaatcgagccactttaagtggtcaca
gggaaagccaagcctccgttgttgcaaccaatttgtgactgcaccatttctggagcacctcttggtgactgtaaggtgtgatggagt
gatggtgctgaactgtgaactggacttttccatctctgtgcttgctagcctcttggccagcctggcccatagcatcttaggcactgctg
accaatagctcgtcttattgaggctttggaagtcgccggtcagggagaagcaacccagccccacaaggcaagtctatccaatcg
gaggctgctcacttcattgcatgttttcttctttgaatcttcacaaaagtttttcagtgtttttatttttaaatacacacccttttgtgaagcccc
aataaaacccagacagaaatgctttgcaaatggggcaggttagtcatgacagatttgcccaagcaagaagcttgattcttgtaaa
actggcatccactcccattctcatttctactcagctcaacttctaattcccagtcagaattgtaaaaatcaaaaagtccacatgtccctt
ccccaagtaaagtgaatttttcatttcccccgatgagatttgttttaatagactttattttttagagcagttttaggttcacagcaaagttgg
gcagaaagaacagagatttcccatatgccctctgccccatacatacatagcctcccctattatcaatatcccccaccaggatggta
catttgttataattgatgaccttatgtcaatgagttttttttattcctccatgcagttcctctgcacccccttcacacactttgggggaaggtg
agggacaagtgcgtgggttctagacttggcctaaccttgcgtgttcagtggcccattctatcccaagatgtcaatctaggcacttctat
ttctcaaaaatattaatactgtgatgtgatgctgtcactttcactgtctccctcactggactggaaatcagagtatcagggctaattggtt
taatccatagatttgactttgaatgatcccattcagcaactattaaggacacatgatgtggcagccactgtgcaagggtgcaaggtg
tggggaaaacaaaatgaacaacagccactgcccagcacctgacactgtgtctgtactgagagcctgtccacaaatatttgttgag
tgaataatactggtatatactgcatacttgccatataccaggcactgttctaaatgctttatgtgtgtcagtcatttcattttcacaccaac
cctataagaaatgtatgtattattggtaccattacaactttataaatgaggaaaggggcacagagtagtttagcaatttctctgcgctc
acatagctgcctgactccagaggcctcaatgagtaaaactggacaagcctatgtttgggaagcaggggtggagagaatgccaa
aatttgtatccaggtcccatggtaaaaattagaatgtgctatctataattgaaaaatatgagttgaattgaattggaataaattgaagt
atagaaatgtccaaaaggtgagagactgataaaaatcacagaagaacgtagagggcatataagaagtatttgacctggaattt
ggaaaatgaaaactttttttcatcatgcaaatgttcatttaatttttttttgttaactagtttgtttattgattatcacatctataaaacatgtcat
gtctatgaaacttcaacagtacacaagagtatttagtgaagactaatttcccttccatacctcatgctggaggcaaccactgtgacc
agtttcttgtgtgtctttccaaatgtataggtcttcgtgtttgatagatcagtacggtgactatagttaacaataatctattgtacatatcaa
aataactggaagagaataattcaaatgttcttagcataaagaaaagagaaatactcaaggtgatacatatcccaattaacccgat
ttgatctttacacaacatgtgagtgtatcagacagcacatgtgccctgaaaatatatacatctattatgtatcaattttttaacatggcag
agaagaaatcagagataaagagggtggggaaataaaacttctctcgacttttcagtgtcctggtgaagagtactagctctgacatt
ttttcataccataagaattaaatctgtagttatttgcataggtaattgctctgatccaaacgaataataaaatttttcccgagaggagca
aatggttatagcctgaacaaggtccctaggtagagcgcccagggtgccatgaagcctggagtcactatcttcctaagcaggcca
gcataagcttgtgccatcattatgcagcatgcaagaaggaatgagccccagaacttggagtcaagtcccaggacttgccataaa
agccaagacatgtaacggactatctggctcctggagagatttatctacctaccaaagtgttggaataaggagcagacctttaaga
cggggaggggggatagctgcctcctccctcttttataggtagggaaaataatttgtccttgtttcttacctatggagtgtctgtttactca
catagagcaattgaccttgctcttatcacatcatcctagggggaagtggggggccaaagcatttactatttactgtgagtcatttaata
agaaatttaactctaatccagtatatctcatgtgcacatttgggataaagttaataaaaatgaatattaaaaacttagccccaaataa
atgggtccgatgggcttgatttttatggaacttgagaaggagcgttctagaaggaggcacaaatgcagaggtaaagggtttcaag
tgttcttggcaagttgtctgttgtacctgaaacctagggtttatatttaaggcacttccatgtcctcagctggcaagtggggaaaagggt
ccccaccactttcttccataatatacctcttagggatactataaaggcaaatcagagtacattctgcttttggagggaggagaacttg
gactctgtgttgtcatttgctcatttttcattcatcccattctgttttattaattcacttgggcaacaaggatttactgagctcctactatgtccc
aggtgggtgttagggatactgtagtgaataaaacagacacagtccctactcttggaagcttataagagtgggggattacaggcatt
gaaccagagttgaacacgtgatgaatgatatgaaggagtgcgttcattgtccattggaagtctgtgacaggaaaacccaaccta
agtcaggagtcaggaaagtcttccctatgaaagagatgtcaaaatggagaccagaaagatggaagttgttagctaggcaaata
aagagtggttatagcctctaaggctagggaacgtacatactaatagtctaagatagaaagacccagcagtgccaaataataaa
aataggactttgactgatggggatacagtttaaaaagcaaacacagacaagatgtcttttctttctgagcctaaatttaccaaaaga
actgtggggtctgtaagttctgttccttctgtacctgaaagaatactgtagcaagatgctaaaagcacatatgaaagtgtcagggct
aggcaaaaaaatataatacaataaaacaaaaagagttatcattaggtagaagcccatcttgtgagagggttggctaaatcctact
attaataatttttgaccaaactccataggcccatgtgaatcactcatttttcgaagtagaattacccatgaaggaaagtgagttggtgt
taacagctacaaatgtttcctcccagactcttttagtaaataagggctggctgaatcacagacacactggaaaacactcatctagc
aggatgtttcaggagcagggacgccactcgaggggttttatgaaccactttaaagcccccacttatttttccaccttgtgcttatgtga
gggtgatctcaagtaccccctccagaccccaacactcacacactcaggtattgcgtcatcattctttatgtgggttgtggggtataag
ggtctcttcctgatgaagttttggttccactcctcatgactgagtgtgcataaaaccactcagcctctctcatctacccctcccttttcctcttcc
tctttctccttctatgttctttcgtttattttatttttttatttttttatttttttggttattccctacctctcttatatccctctttctcctcccccaat
caactccaagttctgaaagcaaccatggcgcaaagagtgtgcaaggttaggtggggaaggagtgcatgggagccattttggggagt
ggtggcgatgggttatggcctgaaaatgggattttttattctttttttctccctatcaaagttggtctttaaaaatcaacactacgctagca
atttttaatcttgttttgaatctcagatccctttaagagatggcatttatggatgtgctcccagaaaaatatgtatacgctcatctatacaa
ctttttatacaaaagtctggaagttcatacttgcacatatggctttaaattttttctcatttctttatacacagaagtttaggttcaggattca
agaagttactcttttaggtactgtgcctacaagtcaggtatgtagccaccaaaggggtcacattatctagacagtcaggcatccata
agtgtggtggaagaaaatccaacatgcttcccagtatattaatgtaaaaacaaccaccaccaccacaataactataatgttcctca
tgtcatcaagcagcaggggagagcactctgttttaagcttaatatattcgcaatattttaaaagacaaatgcctaattgcctttctcact
tttcctcaacaattaagaatttcaatcactctaggccagattttagcccagatagacttctttttcttcttccccaatcactgaatctctagt
ctactattagctgagccctttactgagcaacatggggatttcggggtattttggtgacaagaatatttgggccagtgtgtccaattttcc
aatagctcatcttagccacaagtcagttgtgaaagagtctcttctaggtagctgcattacttaagctgatggttctattttactctctgact
ttcttatcagctagaacaatctatgctctctttgagtcatgggctccttcttttatgaacactagcttatggttaagttcagatatatatatat
gtgtatgtatatatatatgtatgtgtgtgtgtatatatatatatgtgtgtgtgtgtgtgtatatatatatatatatatatatatatatatatatgac
aaacctaataacctaaataagaggctttggtcaggtattatggttttcagcattcattcattgaacagatatttattaaatgcctcctata
tactaagcacatagcacctgtttgtaggtcttggggtcaaaatagtgaacaaaatgaagttcttcctcttgaggcttttgcattctagtg
ggagagacaaaaataaaacaaacaaatatacagtataatataatgcagtgataagtgcagaaagaaacacaaagctatttta
gatagatggtcagaggaggcctcttggaggagaaactgttttgagcagatacctaaaataaagtgaaagaatgagctacccag
gtatggaagggaagaaattcttcagagagaggaacagcaaaagcaaaagttctgagacaggaatgttcttggtgggtttaaga
aacagccaggagccagtgtggccgtagcacagtgagcaaagaggagggcaggaaatggagttggaacagtgccacggact
gggcatgcagggcctttgaagccatatcaataatggactatggttttattctatcggtgctagaaagccacagaaatttaaaagca
ggagagagacaaaataggacatggtttttaaagatgattccatctattgtatgaatgcagggagatcagctggaagaagacggc
agtacccaggcctgggatgatggtggtggaaatgcaggaggtgaaaagggttcagataccagacatattttgaagtcagagcc
aggaggatttgctgttaaaatgagtgtggagtatggctgggcacagtggctcatgcctgtaatcccagcactttgggaggccgag
gcgggcagatcacttgaggtcaggagttcgaaaccagcctggccaacatggtgaaaccccgtctttactaaaaatacaaaaaa
ttagcagtgcatggtggcaggcacctgtaatcccagctacccaggagactgaggcaggggacttgcttgagcccgggaggcag
aggttgcagtgagccgagatcgcaacattgcactccagcctgggcgacagcacaagactccatctcacagaaaaaaaaaaat
tgagtttggagtatgcgagaaagaaaggaatcaaggatgtttccagtgttttggcctgacaaattggctgaattataatgtttgcaga
aggtgttctggaaccaagagtttgtttgctaagtttgaaatgccctttagacctccaagtcctgtcttgtgtaggcagttgggagtgcag
tgaaggttttggttgggagatataaccctgtagcatcccagaaatatgtcagactgtgcaattgggtgagaaactggatgagtgtgg
atgagaatgagaactccgagtactgagatgctccagtatttagaagtccagaagagcagaaggctcctgccaagaaaactgag
cagaggcaacctatataggataggagaaaaaccgggagagtatgttgttccctgagccaaatgatgacagcgtttgaaggagg
gatggatgaactatgtcaagtacccctgagaaagcaagtaagataagaactttgacttggcttagtggagtagacagtgaccttg
acaaaggtggttccagcgagcagtggggaagaacacctgtttatagtgggtccaaggaaaaatgggtctggaaatgggaaaa
gaaactataaacacacattaaagcactttgctgtaaaggaaaacagaaatggagaggtatctggggatggacctgggatcagg
ggagatagttttaatataaggaaactacaagtttatatgttgtgtattgatggaaataacctagtaaaaaaaaacctgataatgtgag
ggacagaggcaattgccgaaacaaagccttgaagtaggtgagtgctccgtggaggaagaggctcgacttaagtgggaatgta
gaccatccatccaggtaggtaggttgatttagtggtggtaataagtggaagttctctttttgtgttttctattttatacttcagtgaaacaaa
aagcaaagtcgtcacatgagagaggagggggaaaggcaggttgtgggtttgaggagagaggaggtgtgaaataatcagcag
caggaaccctcatagtggtttgaaaggctcttggtatttttttttttaaccttgttgttggctcagcttttttggaaaagagaaatacagtaa
tatctcactgtcgacattattaactatctcagggtgtttggagagagaggattccacagtttgaacactgggcttatcacttcctgactc
cacattcctcagatttttctgttttcctcatgatctgaaatgcttcctgggctcatgagctcagaatcacttttatttgctctccatccttcatc
ctgtatattcaatggtggaaaaaaccctggtagaggaattagcagaactgaattctaatcctgactctgccacttactagttaggga
agccatttaacttctctgtgctttttagatgcctgaacaataaatctgagttgataaagacccagtactctagttaatctatacaactcta
tcctaagtaatttgaagatttctattgacagttttgaagtattgagaatatagtggggatcctcaaggcagttcttatagaccacgaag
gacttggcaaccccagggatagccaaagaggaagagggagagcctccagtctgtccttcctgatctgctgacacgatgttgtcta
aaggccttaataataagggactctcttctcctccctcccacagGTGGGCGCATGATGAACTGCCCAAAGATTC
TCCGGCAGTTGGGAAGCAAAGTGCTGCTGCCCCTGACATATGAAAGGATAAATAAGAG
CATGAACAAAAGCATCCACATTGTCGTCACAATGGCAAAATCACTGGAGAACAGTGTCG
AGAACAAAATAGTGTCTCTTGATCCATCCGAAGCAGGCCCTCCACGTTATCTAGGAGAT
CGCTACAAGTTTTATCTGGAGAATCTCACCCTGGGGATACGGGAAAGCAGGAAGGAGG
ATGAGGGATGGTACCTTATGACCCTGGAGAAAAATGTTTCAGTTCAGCGCTTTTGCCTG
CAGTTGAGGCTTTATGgtaataatggcggcttccccagtccacactaaagggccaaggtgctcctttgaccaagaattt
aggtctctcttaaaagcaaagggtattcagaattggaagtaactagaatgatcttctagtttgggggtatttaaacctgctgcatgga
agacgttttaaaggttgacatttttttttccaaattgcatattgatggtagctgattaagcattagttactcttactcccatttcccaaagga
aaggggcacagctccttgtgggctggagggccgatagacccaaggatcttggtttgcaagtaatattttatttgaaaataggatttttt
tctgattaaaagaagttgaataccacagatcaaacccagtctctcctacatgaggacagtgaaatctaaccagaagcggttagc
acatttacacacatttgtgtaggtgtttcactgcactgggggttctggataaagatggctaaaattcagcccacacaccacttgttaa
gccctgcattccggcaccagatcatacctacttggtggaagaagtgccttttggcatttaaacaaaggctttggttataaagtctttta
gttgctgtacttaaactaggaaccaagtccacctgaatccaaggccagtgcttttttgagcctttttaactaccagtccctcttgagtgc
acccagggattgtgtctcttaggcccagagactcatctgaattcccagggatcctgatagccacatggggctttcctgcttcttcaaa
atgacttccttatctctggggatgggacaggaattcccacctaaccagcatttctttgaaattctcaaatatctagagggaaggcag
caatactctcaccaatcctccctcaacccagcattccctttccttcaaacagtgcctgcggaattcccatggccctcccccaggtac
ctgagagtcatttccagcagtggctccaggcacgactgccatgagcgtggaggctgcacatgatgcattttccaaaacggtgtgg
atgccagacattctgtcctttggttcctatgtttcctgtttttgtcacatcttgtgatcaaattcttactttggaaaatgtggtctctgcaaccat
ggcatttttctcaagccaaaggaagagtttggattttgaagtcagacagacctaggttcagatcttaacttggccacttagaagctgt
gagttgtaagatattccacctccctgggacttggctttctcatctataaaatggggaataattacaactagagttataattgttgagaa
gattaaaaaagatgatgaggtggctcacgcctgtaatctcagcactttgggaggccgaggcgggcggatcacaaggtcaggag
atcgagaccatcctggctatggtggtgaaaccccatctctactaaaaatacaaaaaaaaaaaaaattagctcagcatggtggtg
ggcacctgtagtcccagctactcgggaggctgaggcaggagaatggcgtgaacccaggaggtggagcttgcagtgagctgag
attgtgccactgcactccagcttaggtgacagagcgagactctgtctcaaaaaaaaaaaaaaaaaagatgatgaatgtgaaac
accagcactgtgcttgtcctataatagttgctaaataagcaagaatttaccttttatgtggcctatttcatggccttagagtgggatagat
tgatgaggcctatggttataattgaggacctatcactatctcagacacacaaaagcacttactacacacccacccactcactcacc
catgcgcccgtgtacatgcgcgcgcgcgcacgcacacacatacacacacacaccctcccacacacatcacgatagatgaaat
cccaccactaaaaagccattcttttaggtctaggaagtaacaacgtaagccaactaaaaaccatggtggattagttgacagcaa
actccactgataggagacaggagaatagcaacttaggtcaaggacatcaggaagggcgagtggagccctaacaatattggta
gaagaggcctaaaaagcaaattcttattttctattttatcccaaggtggtcttagataggatgtagtggggcatgatggacagtgtga
agcaatagattccccactagaaataaatcacattgagggggagggaaaatgccattaggctgtactttgttctaacaaaaaggtc
aagtgagaattcccaggggttcacttcagtgatggctcccttcctcccactcctgacagAGCAGGTCTCCACTCCAGA
AATTAAAGTTTTAAACAAGACCCAGGAGAACGGGACCTGCACCTTGATACTGGGCTGCA
CAGTGGAGAAGGGGGACCATGTGGCTTACAGCTGGAGTGAAAAGGCGGGCACCCACC
CACTGAACCCAGCCAACAGCTCCCACCTCCTGTCCCTCACCCTCGGCCCCCAGCATGC
TGACAATATCTACATCTGCACCGTGAGCAACCCTATCAGCAACAATTCCCAGACCTTCA
GCCCGTGGCCCGGATGCAGGACAGACCCCTCAGgtgagtacactggtggcagcctgtgtgccaccttaat
gagcatgggctcagtcttcacatggtccaattgctccccagccatggcattcaccttagtaacaatactttacattttctttatagtttgc
aaaagtttaccatgtgcatttagtgagctcactctcatattgaccagggtggcatttttatgctcagttcacagttgacgaaaccaaca
ggaggtgatagagacagacccagagcctaagtctccaaggcgtagccttctctgcctcttgccttgactccaacctgcaaggttg
gctgggtgagggattgcaggtgggagggcctggctcccaagtctatgctccactaatgctggggcagttctacaccgtcaattagt
atttactgatcacctacctggtaagaaattgtgaataaatatgatatgtgatctctcctctctaacaattcattgtaaagataagacgta
aaacattgcttaataatgaaacacaggatatagtgaaggtcctcgttttatggaatggactgtgaatcccatagcagggccaggg
gagtcagaacgagagactaagacctggactgtgaagtttgaggaaaaataataaattgcacagcgcttcatcattagcaaagc
actctcaactgtgtaatctcttttgatcctcacaataaccttgccagataggtgttatactttcattttacagatgaggaaattgacgttca
taccatttaagtaccctcttcaagtttctttggcatgtagatagtggagttaacccctaaacctacatcttctgactgttcttcctctagaa
agaaaagcatcattattctgtcagcaaaaggaagacagaacttactaagtaagcccatttaaccacttggatacaggacagaac
acaggcccttttcactaacagatcttgtgtccctgcctcaggcaggttcagggactgtaggaccagggtgttgctctgcaaggcatt
gcttacaccctgacattctcctctctgcatccactggggggacataggaatcgttccaatgggtcttctgcctacagtagccatggtgt
ccatgtggagggctttcccaggtggtcatggttgagggacaggggaactcagccaagtaatgcccttccacatagcactgcctgt
cacagagactcccctggagtattcccagatggactgctgggaaaatcccacctggcctccagtgtgcccctggaagttcttgaatg
agtctaaccccctgcatgtttcctccccagacatttcatagccagagtcccccgtcctctcacttactggatatgtctggctgcttgccc
accttgcatacaaaccacattcagaggctaccccccagttcagagcgctctccccacccacagtcatttagaaggtgctggcag
gacaagcaagctgtgcagcatagtgggtagcttacagaatgtgaccccggaagtcctagagccaatgctgccctttctacttaca
agggcaagtgcctcaatccctctgagatttagattcttcatctctaaataacagcaactggcgggtcatcatggtttactatgtgccag
ttatgtggcatctcagttaatcctcacagcaacaaaataatattgatagtatccccacttcacagatgaggaaacaaagctataac
atggttaagtaaggtcacacacagagcaagtgatggagctaggctttctgattctggaagcctcataggcattagtgaaagagat
aaagcacttaaaatgccttataaaccataataaaaatgtaatttttattataaaagctacaaaaatataatgtattttttaattgtaaatg
aggaagcagtagcccttacctcagcacagccctctctgggtatagctgccctattagagtacaaaaacagggcactgaatatttta
ccctggcctaacccaaaaaaggggcagaactttcttcatgctcctcaatgtagtttaaaaagaatttaattagggcataccaaagt
gttggtgggccataaagcttatactaagggcactcagctcaggacgttctaagaaaacatgtgaagatggacttacccatctaag
ccactctgaggaccaagaatgcaccagtgggaaccagatttactgagtaggaagctggttcttgtgaggagtggagggacagg
aagcagtagaaacctggcaccacaggaagggccctgtcaggatctcggctcggtttgtaagagaactgtccagccgttcccctc
tcttgggtctctgtttcatccccagtaaaatgaaaagggcgaacaaatgaagtccctcccagagtagacagtccttgattcagtgtg
tgtgtgtgtcaataagaactgccaatagaggcttgccactgtgtatgagtttgctaaggctgctgtaacaaaggaccacacactga
gtggcttaaacaatcaaaatgtattggctctcagttctggaggctagaagtctaaagtcaaggtgtcagcaggattgttcttctctga
gggctgtcagagaaggatctctcccaggcgcctctccctggcttgtaaatggctgtcttctccttgtctcttcatatcatcttctctctatgt
gtatttctgtgtccaaatttcctcttctcataaggacaacagtcatattggattagggcccacctgtctcagtttgctagggctgccataa
taaagtacaacagattgggtggtttaaataacagaaatttatttttctcctggttctggaggctagaagtttgaggtcaaggtgttggc
aggtttggtgtcttctgaggcctctcgccttagcttgcatatggctaccttctcactgtgttctcacctggtctttccttcgtgtactcacacc
ctgatctctctctctctctctttctctttcctggtgtctctttgtgtgtccagatttcctcttcttataaggacaccagttaggttagattagggg
ccaccctaatgacctcattttaacttaataacctttaaagaccttgcctccaaatacggtcatattctgaggtccttggggttagagctt
caacatagaaatttgggggaggggagacaaaattcagcccaaaacatctccctaatgacctcagttttactcaattaactctgtga
agacgctatgtccaaataaggaaatcaacatatgaatttgggaaaacacaattcaacatgtaataaaagtagagatgcctcctcc
ccaccctgccagcctgcagggataggggaccagaccttccctgcctcagaaccagatcacacagctggtttgtggcctgccctg
cgcagtataccagattgcctaaatactgaaaacagaattgtatcagtaccttgatttgtgtttgcgcatgaataacgaatactaccag
ttcttaaaacactgcacttagtttgacaaaacatttacactctacattgtgctaggggccagtacacaaagataaaaaagacatgat
ccttgccttaaggcagaagacagctgtataagtaaacaatgatccgagattagacagtgtgacggatgtaaaatggaaatatatg
caaggcaacaaacaggagggaattattaacactgtctcgggggattagagagagcatcccagagaatgtgacataaactggg
ttttaaaaataagtagaaattacccaagctgatgaaagacattccaggcagagggaggagcagatacagctgggtcttgctctgt
attatcttgtaggtgataggaactaataaagagttttagatagaggagtgacaccatcagtcttgcttttcaaagaggaactccagta
gtatagagaacagactggggcagggaagtggggaagaaagagaaaccagttcctgggggccattattgcagtcatccatcaa
aatgatgggacccgagccaaagcagcaactgtcagttaacaaagacatttttcctagggcatacaaaggaaaaccccagcctt
gggatgaaagggtggggtccgagggtttattagaggttgctttgccatctgtcatcaggacagtgattttaagacatttttcattttcatt
aatgaaaacctcacagcggttaatggtgtgatgagacggaatgcaatgtgatgtgagcactgaatcttgacaggactcacttaag
cgaaactgtgcaaaaacttatatgttccttgaaatctttttctttaggtgacatttgttcaggtcatgtattcatccttgttccaattgccattt
cagtgtgttaatgtctatcataatgaagcatctttattgcaaagtccaattcttagggtgctatgaagtactctggctaggtcatgtgaa
gccagtggatgtgggtcagctgtggacagtgtgtgacttgctgccatcctcgatgactgtattctgaaatagatatggctgtgctaga
atgaaggaatctagaaaggaatgcccctggaagctcatcttgaagagaggatctttttcagcagatcagcaaaccgctggctca
gcacctctgagttagctcagtgaaagaaaaggctgacgcctgccagtgagctccggaggcttcccctttctaacaaggtcatttctt
caaatagggagttcccattgtttcagagtcacttagatgttccaggcactaagacaggtctctctctagggtcttcccaatttagcgag
cgtaaaaacaatggtggaaaggaaaaacctggaaactttgcacagcccagagcctggtcatgggccacacccgctataagg
gaagctgagacacatagctcctagctgagcagctacatgcccagaaaagactcgtattaccacgaaagcatgagcgcaatctc
actggagctagtagcctctgcaatgctgggtgggataggcaggttgtaagtgatttttctggaagctgtgaactccgtaaaaatgttt
acttggatggtcccagaacttaaattagtatatggttcatgaggatccttccccacccccagttctgaatggaaactgccacgaaca
agaatgtatctcttgaagatggcagcctttgctgacagaaccacatgaaaggcaggaaggagatccggcacgctcccaccgtta
cgctaacgtcgcagtatctcctaggtgaactgcatttgtttctcagattctttttagttttctttttcatcttccctaaaaaaaatattaataat
aagattttgggacttgagaagagagagagagagagagacacgcttctgtgtttctgtgacaacactttcagagacaagaaaaaa
aacgccctctggctttttccttggatgtgtgactgtctgccaagttatcacgtttaaaccacagacaataggtggagagggcccagg
gtggagactcgagcaaagcactcttcccaaatggcatgtgagttattgaccagcctgctcggccgcctctaagagcctcgggagt
agggggagttccaaacctctggttcagaaatgttcaggtagcatttctttgtgaatgaaggagtcaggagcttctagaccccaaga
caactttgatttctcagcatcaccatccagagaggcctcactacatgactgagcaaagagaagaagagctggagcttctgccac
aggaaatggtggtttgaaaatgggagcacaggtgaagcgccgatggcacagacacacacttgcctcctggctccatcttgttatt
gtaaagtataagccaagtgggtcacttctccttccctttgattcctgccttgggccattcagcaggtgaccctgcattccttctggtaatt
tttaaacagaaagctacgtgacagtctttttctagatccatttttgtggactctcatttaatttaacttagttcatcgagtgcatattgagtgc
cctcctgccctatattgtttccggtggaatggaggatacaaataaagaataaggtacagggcctaccttcatggaatttgcaatcaa
agtgggacttctacatcttactagctagaaaaatataatatttaaagaaacatattataatcaaggaactgctactagaattcctcttt
gaaaaggaattgtatttgtttatgatagtaccttaataaatgctagaaggcaggtggagaccccccaggaatctgggtgtgggttgg
atggttctgtatgagaatggaggaagatgatacttgtgcagaaatgggaagagaaagagagagtctgaacctgctaggtggtga
aagctgcctggttcacaatggaatttgctccctgggacccttcaatcttcagcagagaacttaaacccacaaaattattggtgtaagt
ttttaaaaaaaagtttttttggtttgtttgtggaaactgattgtattagtccgctctcatcctgccaataaagacatacctgagactgggtg
atttataaaggaaagaggtttaattggctcatagttccacatggctggagaggcctcacaatcatggttgaaggcgaatgaggag
caaaatcatgtcttacgtggcagcaggcaagagagtttgtgcaggggagctcccatttataaaaccatcagatcttgtgagacttat
tcactcccatgagaacaacatgggggaaaccaccccatgattcaattatctccacctggccccacccttgacacatggggattatt
acaattcaaggtgagatttgggtgggggcgtggccaaaccatatcactgatgaagtgactaaaccttgcacccaaggaagcac
agagtagagcaagcagagttataggagcaaagacttagagaaccatgaggaaattactcccagaaattacagaaatcatgtg
cagcttgacctgaacaaactgtaatagtagcacttttttcatacttatccaaatttctaagagcatggggtctctgacatttgatttccat
gtaaatataattaaagaatagcaacaaatggatgagcaccaagtataaaaatacttgggcctactatacaggtagggaaactaa
gccataagtaaagaacagatgggactgaagcatctctggacactggtgaagagactcctttggacttaagatcaaactcattttctt
gtctttccaatcaatcaacaagaatttactgagactctattatgtactgagtactaagagagctgttaaagtagtgtaagagatggtct
ctggcctcctagaacctagcaactatttggagaattgaggctagcagaagtaattgacacttactgaccacatgatggattccaga
tattgctctaggcactttccatacattactatatgggtttctcagaacaacactgtgaacttattgttatgctcattttacagatgaggaag
ttgaagccacagagggtatgagtagcttatctgtagtcacagagctatcaagtggtagacccagaatttgaacttatctatctggctc
caaataccatcactgaaaatggtctgcatggtaaagatgatgttgccaaaactcaggttctaagatacatgacataaaccacagg
tgctgcaggagtccaagggacagggaagaacaagagctgggatggtcaggaaaggtgacacaaagaaaggaagattggc
ctgggcatcaaggctaggcaggcatggtggacagcttagggtgcagcaggaaggagagtatggagtggagcctggggccag
gaatgagcatgtgttggatggaggatgatgaaggatgggttggctgctcagagggcttttgactcaaaaggtttgagtcaaaagg
gctttgacatcactactgccttctttatggggaccgcatctccagaggctaaagcacaaaccacaaaatctgcagttcccatcttatc
cagctctgccactgactttttctatgactctggatactcctgtctgtgtctcagtgtcctcaaaataaaattagtgggttgggtgaaataa
gcactatactatagttcccttaaggttaaaaaggtctatgattcatatttgtatccaaagatgaggaaaaaaattagagtttatgaaat
atctttcaggaccatggccaacttgtctctcagatctagatggactggcagaagcttgtcataggacaaaggtagcagattgctttc
atcctctagagaccctagaaaagatagagagggccggcgttgtcatgtcctgaagccttggtgctgcacccagtcatcgttagtttc
tgtgagttggtgggcagcagagcaaccggcgtcgggcgcgagggagaggaggctgactcaccaggcattactggtgcagttttgtct
tttattttccagttcagaggtactactttgtctgttggttttatttttttaatctcaagtgaaattggaaagaaatattatcttttaaaatga
tataaatggtgggggtgtttcttctcaaatcagttgttgtattggaagttcccaaagtatctatgatagaagaagaaagaggaactag
tcaaaatagtaagtgctactataatggtttgctggatcagttccataggctgacgaaacacaaagttcaggctactggctttgcttctt
atcctagtattagagtgatttctccagtggttcctagtgtcgatatcataaaccttgaatgaatcaatctgtctcaaacacacacatac
acacatacacacacacacacacacacacacacacactcctgcacagagggttctcagtgaccataagtcactcagagtggag
ctgctccttcctccagcatcagcaatgattcaaaatgtcatgctttatacaaattcagaactctctgcctgcctcctaacttttttttttaatc
agagcataagactgttgaagttggtatctggcaaaattaaaacatttaatttaggggatagaacctataaccaaggtgtttgcaaa
gtcagttcagtgagattccttgggctaacttgatgtgtgaaaggcctaaggagaaaaagaatcttttcaaatccagaaggcaactt
cttgccagctatcaggctggaggcccctttggatcttgtaggctgcattttatgaattcattgagactgtctgtatctttggtcaactctgt
aaacatctgattgtgtccaccatgattctttcctttggaacccgactatttttctttcaatttctgccccacaaattcctcacaggttcaaca
acaagcaggcttattccacaatcatccttataagtttcccttacacattaatgttaacatctggtgttactctatttagaaccttagtgcga
atattctacttagaaccctagggcttcagctcggtccccactgttcattaccccggtataactttttccaagcccataagtctctctaact
ctccaagaagtctgtctttagtattcagccacatttctactactaaaccaagctctagttcttgaggttctccaggctgttttccttctccat
aaaatgagaataatgagtgtacctaccttgtaagattattgtgaggattaaatatgttagtacacatgatgcactaaaaatatgtggc
ccattgcaagtgctcaataattgttcattataatcttattgagctacatgtcttgtttactgggggtgataattctcattcactgtttgtcaaa
gtgttgctcctagttcaaaaggatttgataaagtgggtaaaggagagaaaacaataaaagttttctctctgattttgagccttgatgat
tagttctcgggctaattttaaacatgaagatgatttagaggaaagactaaatactttcctttcagttcaggtctgctgggttcaacccag
ttatttgcatgaaaggacaacaatagcactattatgtttatttttaaaaaagataagtagatctttcttcctcccagtgtctcatgagaat
agcgtgaattcacagggacggcacatggaaccattatattctctttacccaaaatggatacaggacacattagcaatcttaagatg
gagaaactgggcagagagattgacttaggagagatgaagataatttaatgttagacatgtggtagttgagttaaaaataaagcat
ttggatagaaaaatcttcatgaaattaagaatgtgaaagtatagtgagagaaattagaataagaaaacagatacaaaaattttca
gtggtctaaagctgacctctaaaaccatgaaaacaaacgtctcccttgggagagaatgcagaaatagaacatgaggctccatta
tcccactttcatgtaagatgtttttaagctcagaatacttttgagattgctctttgacttctttttttttccagAAACAAAACCATGGG
CAGTGTATGCTGGGCTGTTAGGGGGTGTCATCATGATTCTCATCATGGTGGTAATACTA
CAGTTGAGAAGAAGAGgtaggtgtctggcaataaatagattcttatcacactctctgtggtaagcaggggacctctctcc
acaggctcggacttgctctcacaactctggctttctgcatggggccacctttgcaaaaatagtagataaacatatcctgggaccttg
cttaattcagtctaattcaacatgtcttgatcccctctactaggctgtggaaagaaatagaagagccacaggtttctaatgtgagaga
cattattcagataatttcagtttagtgtgactagcactgccatcagggtaaacacaggatgctgaagaagtgaacaagaggtttaa
gagtattcactgggaacagaattcagaaaattattggatctcatccaaaaagtcaccagggttagaatgaaaccaataaggcac
aattattcccctgcagttgaagtgcctagaggtaccatcccctgtcctctcttccaaatttccctatgatacaatatctcagggcattgtg
ctcccctcagccaccttgactactaccaaccaatactggagtcaaaatgtcctgacccaagaccaggagagatgccccggctgc
cttcccatggtaaggatagaacttgatcctcataacactgagctgatgactgatttcattctcaagtagatcagtgtcatctacacaca
accttcttagaaaagcccttacctcagcactctgatgttggttttgcatatataaaaaaatctagatcatagcacagcgacctacttgt
gtctcatttcctccatctaagagttagccaggtaggagggatgggtgattcagatagaaattaggttgacagcctatggggctcgg
ggtagggcaatcacatttagctcatactataaggaaatagtgagatgacccaggatgagaaaactgaacttaacttatccacatt
aacctacctagtaaaattgctgggatcctacgccatactctttcctcaaccacacttggcttatcacatggttgtgctctaagggaata
gtgctccccatcccacaattccccactaccttccccaacacacatacccatcctcacctcaaccccattcaccatttgtcccttgtaa
gttagcaacacacaaaactgcctcaaacttgcggtaaaatttatatttagttgctgcacctttcataaaaccttgctaaagaaattata
ttggcagcttctaatgctataatcatcagaatgcagcctgacgctgaaggcttttcaatttcatgactctttggcaatttcatgtccagg
agaatacactgataaagaatgtgggtataggcattagacaaacttacattcagatgcagattttgctactgacaagctgtgtgatca
aatgacttaacttctcgtctgcaaaacaggggtaatactatgtacttcatggtattgtggtggagattggtatcaatacacagaaaac
actgaacacagtggttcccatcgatgggtgatagatagatagatacatagacagatagatacatagacagatagacagatctctt
agtgtagatgaattaaaatggcaatgtgtaagtgctatggccaggagaagctgcactggaagcatctggaaacaatacctagaa
cagattgaaaatattttaagtcatggtaacataagactttatgcttcaggtaaaagctgaaaaggatattagatactctatgccctcat
tttacagttatggtaagagaaaagacccattgagatgacgtgatttgtccaatgccacacagctaatgatggctacaatgtagatgt
cctaattttaaggccaagactttttccttagagcctaagaccttgctgacttggagccgagttaagcttactcctaaaaacctgttcttg
cactggggaaaataacctgagactaaattatcttggtccaatggtccttttaagcagcaacaatcaacctcacctcttccatctgtct
gaccatttaggactgtccttccagttctacatttgactctgagctgacctgcaagactgaaagtctttgaggactgtagtctgttctctac
tctatttgtagccactacagcacctaggagagtgctgggcaggcatgtcttactttgcaaacactcgtggggactaacttgaacctc
ctctgctacctccaactgcttcttgagtcctcccctccattttacacacacacacacacacacacacacacacacgcactcacgca
cactcctcagtcaggatcaactctgaccaaaaaagcgaagttgaaaccactaggcacaccgtgctcatacccacacacaaaa
aatcccatgttgactttccttgaattcctggaacttcatcagtgtctgccccacatttcctccccaagactcacaccctcacgcagcac
attccaccatgctcaccacatacacactgggcctttcccttccaaagaaaaatgtgcctctcctaaaaatgctatttcctcagagatgtgc
ctttttttttttttttttttttttgagatagattcttgctctgtcactcaggctggagtgcaatggcatgatctcggctcactgcaacctctgtc
tcctgggctcaagcagttcttctgtctcagcctcctgagtagctgaaattataagcgcgtgccaccatgcctggctaatttttgtattttta
gtagagacagggtttccccatgttggccaggctggtctcaaactcctgacctcgtgatctgcccacctcagcctcccatagtgctgt
gattataggcgtgagccactgcacccagcccagttttttaagagaataaattaactggtgttaaaataagtctaccttaaaggctgtg
attttctgggtccagcctccattgcctctgcctggactttgcaataatcccataataaacctccatccttcagtctgccactttcccacca
tccttactgctgcatgatgtatacaaaggatactgtgcaactttagaaagaatgagataggtctactgtgctaacatgaaaaatgtc
ctcaatacattttaagtgaaaagatcaagttacagagaagtgtgtgcagaatgacacctcttgtgtggaaaaaagtctatataagta
tagcaaatatccaaaactgcattgtctaatatggtagtcactagccacatgtggctttttaaatttaaattaatttgaattaaataaaattt
aaaattcagtgacattagtcacagttcaggtgctccatagccccgtgtctgtaagctgtattagacactgcagatatggaacatttcc
atcatctcagaaagttctgttgcacagagctgatctacagggatatacatcaaacttttaaaaatggtttcttcttttttttcccacttctttt
cacaggtattgaaaaatacggtttcttttgggaatgaaattgggttggttaatggaagaaggggatttatactttttactttatactttatat
atttcttcacaatttttattttatgatgagaataaattactcctataatttaaaaagaaagctttttaaaattggctaaaaattaaaatattct
gcaacttattaatttccagagaccctaggccctgagcaaaatttccagatggtgggcaacagaatgacattgttgctttattttctaaa
tagtcccaggtggaacatccctcttacacgtccccccgcccttacctcccacacatcaattcccccagaaatagggaggtgagaa
agctgtgagtgaagcaacatactaccagctggaaaatacaaaagaggtataaacaactagccctgccctcaaagaacttaga
atcctattaggagaccagatatgcacattgagcaacagagattaaagtaattgaatgtacaccaatgagaaaaacacctaatgc
gtattgggcatttgttatgcaccaggcagtgttctaaacactttacaagtggtatctcatttaattatcacaacagccccgtgaggcag
gtatttcaaatcccatttcacagataggcctagagtgatcaagtaactaacctaagacaatatgacaaatgtgcaggggggctgg
gactcagggctttgtttccattgtgcccttggggaaagtgggtatgcaaaggacagtaaagaccaggtctgagtaaggagctcctg
ctggggaccagagggagataaccattatggtttcttttcaccagGTAAAACGAACCATTACCAGACAACAGTG
GAAAAAAAAAGCCTTACGATCTATGCCCAAGTCCAGAAACCAGGTgtaagttctatattttgtttgaga
tgaacctgtcatgtttcctagagtattcctggccagtctaccttgcctgttggacattcacagttttccatccagagcagaggaaggta
gggaacaggagtcaagaacaagagttctcctaaagtcactaaacgtcagtgtttgaaataatgggcaacactggataattttctg
gtcatgagtcttcacaggaaaaaaatgaagaagctggaaatacatactgtatgactctttccagctctggcattgtaggagtctag
gttccatgttagtcaattatttccttttctagggaaaagagtgcaggcttgaggagagaggaggtttggaaaagctattgtgtgacat
gttggactgatccaagtttaggatttactaagtgcaaaagtgacaaggaaggtaggatcttcaaaattctagctagagtgtggttaa
agagatgaaagatgagatggaagaaagaaaactgtgacagagtgatcactggactaagaagtgaaggatggaaaaactgg
atgcatggtgaagttgagaagcagatatgcttgaaggaagggatagagacgctaaaaggatcgtggttagatgtagagacact
gtagtttttcaacatgaaggcaattcttggtattgtataggccagaatctggacatttggggtgtaggtagaggcaaattcttgagtaa
aggatgtgaaggtaaagatggttttgatagtaccttagaaaattgcatgaaaagacagcaaatgcacttctgagaaccaggaga
tggactcttgaacaaagttcttatttctgctgtcccctagtggcctggagggcttattacacaacccagctccatccttcccccaacta
aactccatttaaatagatgagaatcccaagagtaaccctttcaccccacgctctcatctgcctgtttaggtaaccaggttcaccttga
ccatagtgtcttccctcactactctatcctatgctgctagcatccctcttttttactgtgaagcatgacatatggtagtcactagccacatg
tagctttttaaatttaaattaatttgaattaaataaaatttaaaattcagtggcattcatcagttcaggactgtcctcccagttctacatttg
agtctgagctgacctgcaagactgaaagtctttgaggactggagtctgttctctactctatttgtagccactatacacctaggagagt
gctgggcaggcatgtcttactttgcaaacactcgaggggactaacttccacctcctctgctacttccagctgcttctaatcacactttta
gtcctctcctccattttacacacacacacacacacactcactctcacatacacacactcatgcatacccactcctcagtaaggatca
actctgaccaaaaaaatacacaacacattaatgtcagctcagtgagttacccttaaacacatatctcgatatttggtaaagcaagtctt
cctaatttgtttttctgcaaaagtttttggctattcttgttcctttatactttcatatgtattttagaatcaacttatcaagtaccacaaaaag
aaaaaaaaatattagaattgtattgagtctacagatctatatgaggagaaattacatttttcagtgttgcgtgttttttgttttttgttttttg
ttttttgacagagtcttgctttatcgcccaggctggagtgcagtggtgtgatctgggctcactacaacctccgcctcctgggttcaagtga
ttctcctgcctcagccttccaagtagctgagattacaggcacctgccaccacacccagctaatttttgtatctttagtagagatggggttt
caccatgttggccaggctggtctcaaactactaacctcaagtgatctgcccacctcagcctcccaaagtgctgggattacagatgt
gagccactgtgcctggcctcagtattgagtcttctaataccataaaactaccactcagatcaaagactagaacattgcccgtacttc
ctgaaggcctcctgtgccacttcccaatcattacttcctctctcctccccaaagataaccactatcctgacttctagaaaaataggttagct
ttttccttttttatttttgaactttataaaaattgaattctttattcttttttctctcatgtctgatttattttgctcagtattatctttatgagattcat
atatgtctttgaatttagatataatgcattctttttcattgcttcatagaatataaacgtatgaatatactagagtttatttatccagttgactat
tgatggacatgtgggttatttccagtttgaggctattatgaaagttgcagctgtgaacattcatatgcaagtcgttaagtggacatgtgc
acatatttttttgggtatatacctagatatacctggaagtagaactgctgaatcgtagagtatgcatacctccaaattgactagataag
gccgagctgtttttcaaagtgggcgtatccatttacttttctatcagctacatatgagagtctcaattgctatgccttttttttttaaattttttttt
gagacagagtttcactctgttgcctaggctggggtgcagtggcgtgatcttgtcttactgcaacctccgcctcctgggttcaagccatt
ctcctgtctcggcctcccaagcagctgggattacaggtacgcaccaccacacctggctcattgttgtatttttagtagagacagtattt
caccatgttggccagggtggtctcgaactcctgatctcaggagatctgcccgcctcagcatcccaaagtgctgggattacaggcat
gagccaccactcctggcctcaattgctatgcattctaatgaaaacttggtattaacagtctaattttagtcctactgttggatgtgtcttat
tatgcttttattccacatctctgtaattattaaggaagttgaacaacttttcatatgtttattggccatattaaaattctttcttaaagtgcccat
ttaatctcttgcccatttccctttgaggtttagtctttcttttatggactagtatatgcttttcatatattttggatatgtgccctttggcagatatgt
tagcaaataccttcacccatctgtagcttgcctttggaatttctcagagatacctactgataaagagaaggtcttaattttgttgtagac
caatttagtctagtcctttttaagcattactggattttatttgctaatattttgttaagagtttggttttccacttatgtttctgagtgaaattggcc
tgtaattctcttgtataatgcctttttttttttaagaaggcactgcagtggctggtatatagcattcttgtgaatatatctaactgggatacaa
gttgaggtagaaatatttcaaatgtccttaaaaaaataagtaacagagttcttcctggactcttctttaatcacaagcctcagattgatc
ccaaaatgacacacagctactctacctaatacccacatcacggtaaagttggtcgctctcctgttaaaaattcagactttaagaact
ggaagggacctgggtagtcatgcccaaccagtggggttttgatataaagatttatgctaattcacataaggagttggggtatatgtta
gtttcctagggatgccttaacaaattactggaaacttggtggcttaaaacaacagaaatttattctctaacagttctggaggtcagaa
gtccaaaatcaaggaggcacatcctcagggccacactccatctggaggctttaggagaaaatcctctttgcctcttccagcatctg
gtggctccaggctctccgtggcatttgttggcttgtagttgtgtatctgcaatttttgccttcatcttcacatgacctccctctctgtgtcttctt
cttttccatctcttataaggacagtcatcatcagacttcggacttattctaatccaggatgaccttattttgaaatccttatcttgacatctgt
gaagacctttattcaaataaagtcacattttgacattctgcctggacatatcttttggggccacagttcaacccaccacagggtgcatt
tcctttttgttattctctgcgatatttgggtaggatgtcttatttctccccttaaatatttgctagtagagcaaattgctagtaaagctatctga
gactggggtttctttggtggaaatttttttaagttatatttttattattaaattttctcctctacatattagtgaacaaacttttttcagtctttttagtg
gctaccctagaaattataaaatacaactttgacttaccaaagtctaaggtttacttccctcctgcataatacttcaagtccacaataatt
tcacctcttgatttaagtgaccgttttgtcattatttgaatttcatatatattttaaacacacaagacataattattattgttttatatatataaat
atatacttagacttacccacattttcacaattttctttgttcatatttgcgatgtctttattatatcaatataaagactgtaataatgtagaca
attatttaaaaactaacaatgcctttattcttatttttaatggctataaaataatcttataaagaatataataacatgaaaatcactaaac
aagtgtttactgtgtgctaggaactcttctaggacttatcagagctagtatcttgcagaattaattccagcggccaccattcacaaaa
attatgtgaaaataatgcctctggagttgcttgtaaatgatgctccctaaagatgtacaaatcagtggtcctaacagaagataataa
gatacaaaaatatactaacttattatatttatgtttaaaataattccctatgcctggataaaaatcctgaagtgaacatttaagcacac
acagagtcttaataggactatgggtgacttcttttacatatttttctcctttctaaaacttctgaattaatgttaaaaatgtaagttatttgcct
ccttctgcctctaggtcaggttatgctaaagttctcccaaacaggaagaccagcagaggttgcatctgttgataaaggtctctcttcttt
tttttttttttttggtgatgcggagtctcactctgtcgccaggctagagtgctgtggcgccatctcagctcactgcaacctccagctccctg
gttcaagggattctcctgcctccgcctcctgagtagctgggattacgggcatgcaccatcattcctggctaatttttgtatttttagtaga
gatggggtttcactatgttggccaggatggtctcgatctcctgacctcgtgatccacccacctcagcctcccaaagtgctgggattac
aggcgtgagccaccacgcccggccaaaggtctcttaataaactgttttgatagcctctttatctcatcactgcaagaaattctttctta
aactcaaaatttcttcaaaatgtatttaagaattgtatgggatcttgaaagccatctatctgaaccacccaattactgcttgaattatctg
ctacaacattacttccaaagtgttgcctagctcctttttactgaaatatagtttgtgaacaagcagcatcagcatcacctgggactttatt
agaaatgcagaatcaggccctgctccagatcttccgaaatagaatcaaccctttaacaagatccccaagtaattcatatgcataat
aaaagtcagcagcactggtctagaccatgcccaagcacttataactataggagctcattgcctgccaaggcaattcatcccacat
ttgaacatcttttaccattagaaagatcttcattatattgaatcaaaatattttccccaaatcctaatcttggtttaaacctgagatactttat
aggcaaattgaattccttttctatatggcaattcatcaaatatatgaagagaaaaattatgtcccatccctttttcctccaataaactttc
ctaattccttaacccttccatcacatgacaaaattccaagttttctcgcattaaaacacatgtggtgtggcttcaagtctggctctatac
ccagggagagtggacagcagcattatcccataaccagtgtccccaaaatgtgttgaattaatgacttccctattgtaagtgatggc
atccgcatcttacaaggatgtggtctcaatttattttgaggtctttgtccaggaattgtggattttaattcgttcaaagtaacatcaacaa
atatcagctgaagagtttatttttatgtgtccaatactgttctgtggggagtacaaaaatatatggcttatttctcaaggaatgtatagact
ggagaaacaacacataaatatatcagaatgttttaaatatagcatagagtgccataaagtgtaaattagactttaaatacctgagg
aatttaagtaagggataggtcattataagttgagtgaccaaaaaagaatagtgatagaaaggacatgcaccttaagttaaaaga
gcttaaggcaggggactcccggctttgacacttcttgcttgtacacattaggaaaacatttggtctttctgagctgtagcttcatcatcg
gtgaagtgtgagtaacaacagtatttaacacagagtggttgtgaggcaaatgagaagacatatgtgaggaggaggaagagtag
gaatggcagtgtgggatgcaagaattctgcatgtagccggtgatatgagtgagacggactaaattctgttgctattctgtcccctcca
gctgcccctgtaagagccacaccaatttcagtttcttgtgaggaagacatttaaaacatttgagaagcactgacaatggatgaggc
tgccttgggaggttgtgcaccccaagacgccacttggggagtccaagcaaagcctggggaattgagttccagagaatttggggg
agaattcccacctgagaaggaggttggaccaaatgactggaaggaactttctgcctcaagtcttcttgagtctgtgcttctctatcgg
agagttgggtgagaactagctctctctgttcagctaatctgctttctttgcttctcttgtagCCTCTTCAGAAGAAACTTGA
CTCCTTCCCAGCTCAGGACCCTTGCACCACCATATATGTTGCTGCCACAGAGCCTGTCC
CAGAGTCTGTCCAGgtgaggcatctctctgcctactctccgtagagagggaatacatgaaggaggggaaaatgagga
agttttttttttttaaggtgggaagagggagaggatcagggaaaatagctattgggcactaggcttcatacctaggtgacaaaatac
tgtgtataacaaatcctcatgacacaagtttacctatgtaacaaaccagcacatgtacgcctgaacttaaaataaaagtaaaaaa
aaaaaaattaaaacaaaaacaaattaaatgaaacagattgatgagtcctggactggggaagggaggccacagcatgcaggc
aaaaaggagtctctgtggctttggttttccagtttccatgaagcccccaatacctgctcacacggggccactgctaaccccctgctg
gccagtgtttccctgagagttgtccaaggaccacatcagaatcagccagcgtacttgttaaaaataaagattcctagggacttcca
cctaggattctgttaaatgaaaatgtctatggagagtagccatagacctacgtatttaaaaaacccacaccccaggtaattctgata
cacactcaagtttaagaacagcagctggagtccaggagttctcaactccagctacaaaacagaatcaccagggaagcattgta
aaaatgctcatgcctagactctgtgcagccccatttaatcagaatatttaggggtggagatctgcataggtgttaagcctagaagag
aatatggggtgcagctcaaaatgatacttgcatattctaccctattgcaagatcagcagggactaagtttacttcggacaggaatctt
tcctttactgaatgaatagaaataaattctgggctgaaatctttgctccatttgggctctttcagaagagagcccaggatgatagagg
cacaaaggtcacacaaatgcctgcatccaccttatttttcaaagctcctaccgcacacacactcatccagaaatgcctgggcagg
tgccctatatttcaagatgaaaccaatcttcaacttgaggtccattctcacttcactgtcatatctaagaaggaagtaaaaatataaa
cctgacttcaaagcttcaaaaaaatacatagatttttaatgaagtttacttaaggacaaaaacagtatgctatagttaacattttatgg
caaaacccttaaattctattttctttgtttctttgacatgagagatctttgcgcataaccctcttctccccttcctctctcctgccaataccact
tttctcttctccctttgagtcccactagactttttaaaaactcaataatttacaactctcttggcttcccagattgtgacccatatgtaacag
caaaacaaatggttttccttacaaggggatggaaggggagagggcaaagagggagacagggcactgagtgctggtcctcag
atcatgctccccataatagcatgcttatgcttggaagggagctgtggcccttgttgcaggtggagaagcagtgtgggaacccaagt
gctgtcccagcaaggccctgtctgtgacagaccctgcacaagccatgatctctaagaccctttccttttcctcagcagtgctgttttca
tttgcattctgtgaagtgagtatccagtccctctactcacagacttctgctttgtccccagGAAACAAATTCCATCACAGT
CTATGCTAGTGTGACACTTCCAGAGAGCTGACACCAGAGACCAACAAAGGGACTTTCTG
AAGGAAAATGGAAAAACCAAAATGAACACTGAACTTGGCCACAGGCCCAAGTTTCCTCT
GGCAGACATGCTGCACGTCTGTACCCTTCTCAGATCAACTCCCTGGTGATGTTTCTTCC
ACATACATCTGTGAAATGAACAAGGAAGTGAGGCTTCCCAAGAATTTAGCTTGCTGTGC
AGTGGCTGCAGGCGCAGAACAGAGCGTTACTTGATAACAGCGTTCCATCTTTGTGTTGT
AGCAGATGAAATGGACAGTAATGTGAGTTCAGACTTTGGGCATCTTGCTCTTGGCTGGA
ACTGGATAATAAAAATCAGACTGAAAGCCAGGACATCTGAGTACCTATCTCACACACTG
GACCACCAGTCACAAAGTCTGGAAAAGTTTACATTTTGGCTATCTTTACTTTGTTCTGGG
AGCTGATCATGATAACCTGCAGACCTGATCAAGCCTCTGTGCCTCAGTTTCTCTCTCAG
GATAAAGAGTGAATAGAGGCTGAAGGGTGAATTTCTTATTATACATAAAACACTCTGATA
TTATTGTATAAAGGAAGCTAAGAATATTATTTTATTTGCAAAACCCAGAAGCTAAAAAGTC
AATAAACAGAAAGAATGATTTTGAGAtctctgagttttgaacagtggactggaaaccatgtaagagccttaaaagt
acagttctgtgcaaatggcattcagttttaaagaaaaacgtagcaaatgtttgatggtgctgttacaaaggagcttggaatactcag
aggaacttgtcccatggtgatttttcacttctcaaaatgatgtttaaatcccagttctctgttgattcccttgaacaacaaacctggaacc
tcagctaagactctctgtgaccagattctgaacctcttatatccagggcttcaaggggtattgcaggtcaaggtctttcctaggcacttt
ctactccctgcatacctctcctcacactaaatttatcctctagtagaaaattaagttattttggtctaacagcttcaaatctttgaatgctc
aataacttattttgcaagctgcaggcagaaagagactttttaagtaaagtcctttgttttttcctattctctgcttttagacaggctgtcctc
aatttaagccctgctttttcttattgtttcttatataaacttggtaagtactgtaagaaacagccactatcataccattgcataataaggag
caccaacttcccagctcaaaactcaggtccttattgccttgtatcttacctcctctatgaggtcaattcacattgtaagcctgttgcttagt
gcatctcgtttcctggtaccagcttctttaatagagttcttagttgcaatcaacagaagctggctttggcttttttatgtagaaaaggaac
ctattgaaaagatactgattggttccaataactgctagaagtttctgcaaaaccatgctttgaaagtgagcaggaaaagaagaga
ctaggctgtggctgggagcacagccaaaattacaaaaccagcccagggatgatgatcctgttcatgcacagccactgtcccca
gcactaggcacagactctaccactgcctcactgtctctgctggacttggaaacttgatattactgttactgctgcactgtctgccatga
aaatgaattctccagggtcccttcttcatcctttcatctctagcttataattcaaagtctgggattgagtggccaatcctaggtcacatgt
ccatgtcctatctccaaggggggctgggaattgaatatctggcattttccactttcacttcttatgaattaaggaattctacaaataata
gaagtgggattcaggtggtaggcagacaaaaaagcctcacaattatccactacgccacccttgtataaccttaccctcattcactg
tctactctcaaaactgtggagctactaatgaagatttgtaaacccgggcttatgagcacccattcctttactacaactcagattgctct
agaagctcagttcccagcacttggatttttccagtagctgaattctacctgaaggaagggcagaaacaaagggtgaagaagag
gctatcacttccaagtatcctgcacccctgggctcaagacctcactggggagggagtcttttgggccacccaccaaacagcactg
gcattatgcctctcaccctagaccatggttacacgtggtaaaacaaccccttctggtgatacattcacaactctctagtttcccccaa
atggcactatggggagcgggagcttgccttttcctcagacttaaaacaataagttttccccgtgtttcccctctaatgctgttttcttttga
ccaagcatgtctgaattctagagaagtcaggaggaacacacccattctcggtttgaagggactgatgttctgaagtacaactggg
cacagtcccaggctcttcaggacgcttcctccattcacacagcggggatgtgattgttacagcgggtggtgtgtgctggctgagaa
gccactgtgaattgattcttcttctgaagtttatgtttctactttttggaaatgaataaattacagccagtccatcaaggaaattgcaat
CD86 genomic sequence

SEQ ID NO: 2

gcaagagcactgtccctggctgtggtgttgtttctctagtcagttcccctttctgtatttgagttctaccgtcagtcctggcattatttctctct
ctacaAGGAGCCTTAGGAGGTACGGGGAGCTCGCAAATACTCCTTTTGGTTTATTCTTAC
CACCTTGCTTCTGTGTTCCTTGGGAATGCTGCTGTGCTTATGCATCTGGTCTCTTTTTGG
AGCTACAGTGGACAGGCATTTGTGACAGgtatgtttgtggaggctcagacgcctagggagtggcatgagata
aagctgcaagctgcatctggggcagaaatgctgatgtgctaatggccggccagagaatgagtaaaagggattgcagagagca
tgcttaaaacctctgaccatcaggtttgcttctcagattgactacattggaggtgggatattacaaaaatctgtctcttcctgccagatc
ccttcatctgtttttcgtgagctaagagacaaaataggcaggaaatagaaggtgccacttaccaaataattggcagctgttcttggct
ttggggtgctggggtctccgagcagcctctgctctagaagaagcagtccaaagatgtcagctcgcctcgcctgagtcccctgtgcc
agtgggaaatccagagaagggggatttcctcctcttgcagcctctctgcaatggacttacttggctttcctgtttgacctttcccttctct
ggtccagagacccttccccaatatttcttcccatccaagtgccccatcccaatattagccccacttggcaccagagaccaagatct
aatttaaaaagaaatattcttgggtcaaaaaagagcccaagcaagtgattgaacataatgtgtttcacatacggtgaacctatttgc
atttgcatttgcaaacgggcttaaaatatcatctctattaatagcaatttaaggttctggagagccaggtgaaaatagtttttgacaaa
gggaacttcctactccccttaaactgtaataatgaaggaaatgaactgtttatcttacatgtaacctcaatcttgggactaaggccct
gtactaaaatgcgtctatttatgtgctcagacttgcagttcgtgttatgtctgctgctgcagataccgttaatattatttatgtgagctatcct
gtgtataatggaagcttttataaatctctatttatttattcctaatatagttattaagtgcttgctatgttccaggtactagggacttaacagg
tagcataaaagacataaggaaaagctgcactcttgttttctagcctagtggggaaatcacattaatttaatcacactaaacatgact
acatagcaatagtgctttaaagggaaggaaattgttctatgtgactatatcagctgattaattaccaagcctttgcatttgatattttggtt
agtctattcttcttgaatttcatatgcctcttcctgggtgggggtgaggatgggattttatggagttgaggctagggcaggtagggaga
aaacatgagaaagatgaagagataagccaagccagattcttcagcagaaaaatcaaggttgaaataccatgtttcaaaaatca
gactgaggtgggagttgaggttaggggtccctaggccaggggattgaagcttcaaagagataaaactagagcaaaagcaagc
acagagagtggcagagaggtccctgggcatttttccacagtccattctagtgctggcaatccacctttcatggccaggcaggtaag
agtatttgtggggtgggagaaaggacagggccataggctgggcacacagccctttactggcccttatctctcctctcttctcctatac
agtgctgtttccgaactgtacattggcttacactcgggctgaggtttgggaaataggcgccattttgaatatgtgtggaggaagaaa
agtgtgtcttcagcactttccacctccccatcacggccctgagacctcaacaccgggaagcatctcgttccctatcggtcctcctttat
tcatggacggatatgattcctttctaagttccatgtcctttttagataaattaacttgaacctaatgcctaatggcttaaaaacaaacaa
aaaaaaccctcttccttccagctagcatttgcattttaacaggggctttcaaaaaatgccttagcccaaggaatgagtaatgtggga
attccaagcagcagggtaggactggtgcacagtatggggagagaaggcccctcaagttgtggccctgaaatgttggcttcctctc
tttgaccatgatgctgtttctgagaaaacaagaatcaggctaccttaggggaccaggatgggcatggctcccttttagtgagttctat
gagcctcatacctgacagtcagagccctcgagtggatgagcacagactagaagaagcactgtgaaactttgcatgatccttacct
ttttggcaaaaaggaaaaaaaatcgttctcaaattcatcaatagtttgaaatagggtgtgccttgattcagaaagtttcgattctagat
acaactcggagaactaggcgtgtcttgtacacagatttgctcttgggggaccggaaaagctaaatgctatcgccatgctatgctcct
tcttctaggccagtgaggggaacgcattcttcattttaatatttcagttgcctacaatattggaaggtggataaaagcaccctctgctc
cttctaaatctgcgaagacatttcttctctgcacctactcatccttgatgcagcttcctcatgtctgtatggaaacactgtgctctcaaatg
agtttcagaaagaacaactcacgaaagaaaacaagcattcggtcagaaaaatctccacaaatggggaataagggggatttgc
tccaaggagagactggaaaccaagtcagacataaaatccagcctaagctagaaggagacatggctggtgggagcttgagga
aaacagagctcaggatggaggacgtctccacctccagtcatgtcctctgtccaccagacaccaagaagtgttcatgttccatcga
ggcagccctcacacccatcccttcctcatcatgccgactgcctctttactgcttcaggctcacatctcaagtcgacgagcctgtaata
ctggctttcttgatcaccctgataccagccgtcacctcttgacaggcttattttctttaagctgtcattacaccatttttctgctcccaaact
attaattccaaacttccaattttctgttaaattaaatatgaattccttatttgactttccatgccctattaggctatcttgctccttgctttacttat
agaaactaatctcccattatttatccaaagacaacctctgctgcaggccagtcagcttttcttactgtcctgtaaaaattccatggtca
ctcctccatttccatgtgtccttaaaaactgttatttgattgtgtctcagaaagtcgtcaaagaatatataccaatgaaaagcatcaaa
aaggttatacttgatgttatgtgtgtatcaaaaatatggctgaaatatttatccagtgaaactcaatcaacactaaaaagtggttctttc
ggaagcatcagttctttgagacccattaaacagatgcctcggatgcagggttatatattatcaggaatctgtctagggaagaattatt
ggaagcttgcaaagcctttcaaggacagaggacgatagctaccacgttgagttctaggaaattaaccattgttattgttaaaggaa
gacagcgtttctcagaggaagactgttaaacagtgcagtggcccaggctaacagccctcataagtgggagtatcagaatgagtg
gacttaattacttaaaaccaatacagggtggaacttcatctgctataacagaaatcaactcgtgcaagttctaacatgcagggtac
agttctgagaccaagtctgactcacctgtcaaagctcagctcaactattaccacctttacaccacccttccaagctgtaggagtgctt
gctgttctccatgtcttctgaagccctggatcacttgtagccagctcagcagactctacccagacagggatcctttaaatgtaccatat
tgtctactgtgttaaaaatgagaggaactgactcagggtgagagcgatggagtgtccagatgttctcctttatttctccttattcctgga
aatgtaatgagaatcttagaggtgaactgaaaagttatgagttcaaccacttactcaattcgagattcgctcctaaaatgtctcttctgt
gttatcacccccactttggtttgaatagtacttgtgacagggagcttatcacctcacaagaaaatccagtcattgcttgtagctctctatt
aaaagttttccatcatctggaactgaaatctggctccctgtaacttttagttattggaactacttgcccttcagcaacagtgtatgtatcct
cccatggaagggcccttacatatttgcagacacccagcatatacttgcaatcttttcttcttcaggttcattaccctagtccttttagttgtt
cttcatttgacataatttcattattcactagtgaaccttgctgcccttccccttgataaaccgaatttgtcagtgtcattcaagtataactga
cctcacagaacgtgataccacaagcgatgtggtctgattagcacagagttcagtgaatgaatcctacactaggattggatgaaatt
tacttagccataccacactaacacttatgtgatttttatgtttactatggatagactatttctcctgtgtccacttcttcctcttacacagttgtt
atttcaaaactgaagtacagattcttacacttaccctcaggagattcatcatgttagtattagtctctcttttcaggctttatgaatgttaatt
cagctaactcatttttgagctatctgtctcattttgtgccatctgcacagcataagtttgatttctgttgcttttattagtagttttactaaatac
ataaaagtgaaatagtgaaacacagagtcttgtagcatccactgtgggatcagtcttttagacaagaatgatgcagttgctgagtc
aaatgaataaatgaataaatcaaacaatactttgtcctcatttcccatattgatctatcaccatatcctgttaattataattctaaatatttc
ttgatctatccacttttcccttacttcacctgctactatcccagaccaaacagccatcttctttcactcaaacaattgcagtagccaactg
attggtcttcctgcatctgtcctggcttccctatcatccatttgctacacagaaaccatggtcatcttttcaaaatgcaaatctgatgatat
cagtctcagctctaatttctttggtggttcacatataaagactgaaatctttaactgaccaataacacacgtgtgatctggcccctgctc
acctcttcagccttgtctttcacctgtctcttcattttggccacagggacctcctcgtaccttctctcacgtgccctcctgcctcagcgcctt
tgcatatgctgttccctttgccgagaactcttcctgtcaactcccaagcccttcacctacttagcacctacctattcaatctgttctgtttgc
ctcttggtatgttacaaactgtctccaaacttagcagcttagaacaatgaatcctttaccctctctcacaatgtttggggtcaggaatttg
agcgggccttggctgatttttctgttcctcatgccatcaattgatatcacctgatgttattaagctgatggatgggctgatctggagatgc
actgtccagtttggtagccactggttacctgaaatgcagccagtcctaattgagatgtgctataactataaaacacccacatgattat
tgaagatttggtgccaccaaaaaatttaaaatattcgttaataatttgtattctgattacatgttgagattataatatttcacatacatcag
ataacataaaatgtcattaaaattaatgtcacctatttctttttaatttctttaatgtgactactacaagttttcaaattatatctgtggcttgta
attgtggcttgtattgtattctttttttctgagatggagtcttactctgttgcccaggctggagtgcagtggcgagatctctgctcatcgcaa
gctctgcctcccaggttcaagtgattctcctgcctcagcctcctgagtagctgaaattacaggtgcccgccactatgcccagctaatt
tttgtatttttagtagagacggggtttccccataatggccaggctggtctcaaactcctgacctcaggtaatctgcccacctcggcctc
ccaaagtgctgggattacaagcatgagccaccacacctggcctgttttatattcttactggacagtgctgatctagagcaggagtca
agcagttttttctatgaaaggccacatagaaaatgttttcagctttgcaggccatgcagtctccatcatagctgttcaactcttccattgc
actgcaaaagcagccatagataataatttacaatagacatagcagtgttccagtacaactattaataaaaataggtggtagccag
atttggcctacaggctgtagtttgctgacccctgatctagaagatccaagattttattcatatgtctggtggcttggcagggataggtgg
aaggctcagctgggaccattgacccaaacagctatacagtcctctccagcatgatggtctcggggtagtgggacatcttacgtggt
ggctcagaactccagataaggtactcccagagagacaggtagaagctgtgaggcttcttatgaccaagctctcgaagtcccaga
atatcccttgtactgtattctatggtcaaacaggtcactcaggctagcccagattcaaagagaggagatccaactctacctcttcatg
ggaggaggagtagccaaggatatgtgtttctttttaatctattatatcattcttcagatctcagtttaggctggtcctgttatgggctctca
aagtaccatgaacctctcttttgtagcacttgtcatagctagttttacatttctctgtatgattacttgatcactatcttgcttttctactaaact
gtaggcaaccacgtgaagaggaactgtttctggttttgctcattatattcctagcaccaaacacaatgcttggttcaataaatatttgtg
gaagaaacgaatgaatgaatgaaccaatagcaaatgaatgaatgagtaataactgtatcaatattaatcctacatttctccatattg
ctgtcacgtatatcataagatactctgtcagaagccttgctaaaattcaaatatatttgattcccagtaaccttcttattttgtagttcaga
aactttataaagaaggaaataagcctatcttactcttcccagtatctcaaagagggtttctgccctgagctgctcaagagggtttctg
ccctgagctgctgttcattctgcaaacactgctcgaatacccactgtgtgccaggtacagagagttcttctctgctgtaatctggaca
ggcaccagcttcccagcgtgggtttaggcttcaggtgcacactactgtgtaccgtctaagccacacctagaagagctctggggaa
atatgactacttgggcagaaaaggaaggaactaagaagaggtatctttgtgtctgaggtctgaaggagcgtgtgggctcttgttca
ggcaaagggcaggatgaggggaggtggggtggcagcagccagtaatggggtgggacagcggaatgcagaggatgaaact
tcaggtcctggtgctctgagaagtaacgctgtgcagcatgtcacacccagaggcaaaccaaggccccagggagctgatgttgc
actggagctctactctcctctcagcgagctggtgacgtgccagtccagcaggcctggcttatccaaccacaagtatgaatcggca
gaaggcaatgagctgggccctgagtgctgctgggctgaggccgacctaatccttcctccacagagactgtggtgtcccctgctttg
ctcagggtaagaactcttgtatacctcacaagaagccaaggactacctaccaccttccacactggccctggagcctgcattgtagt
tatttgtggacactttttcttctctttagtgccaggtgggggaccaaggcctacatgtctttacaacccctcaatctctagaacaagtctg
acactgagtagatgtagcaaatgtttgcctgaaagactacctcaataaataaccttctgaggcaccagcaaacttctcagcatttttc
ctgatactccggttaccactaacattctacacaaagttgtgaaataagtctttttctttgttgctctccaacatctactgtggacccctcct
ctcacttcctgtttcatcctctctgcactcccctgtcccaccccattactggctgctgccattccacctccctcatcctgccctttgcctga
atgagagcccacatgctccttcagacctcagatacaaagataccccttcttagttccttccttttctgcccaagtagtcatatttcccca
gagctcttctagatatggcttagatggtccacagtagtgtgcacctgaagcctaaatccacgctgggaagctggtgcctgtccaggt
taaagtggagaagtactctctgtacctggcacacagtgggtattcgagcagtgtttgcagaatgaacagcagctcagggcagaa
accctcttgatgcaaagggatactttggggccccttcttctcccaccccagtctgtctctctgagagtcctctcgattccaggagccac
catcacacctggccctaggctgtgctgctcccgtctgtctcagaggctagataacatcagagtcctttccactggctcctgtggcag
agcaaaaactggttggcatttttaaacgtgctacaccagtgtgtgaaagaaacacaggctgcatgggtttaaatctcagctgtacc
atttactagctgggcagcctagggcaagtactgtgacctctctgagactccattccttcatctgtaacatggggacaaataatctcac
cctgttgtgagcagtaataatatgattaatcatttagccaactcttattcatgttctctgatgggccagacatacaaagtaagtgaaag
tggattacggcaggtgctcttcttggtttctggagtgaacctccatttacatggaggctcctctttttagatttctgactagttcacccacct
tattcatagaccttattctgtgcttagctgacagaaatctcctctcagagaatccccccggtaaattcttaggttctttcctcttccattccc
ctttttgctctctccctccgaaggcaagagtttccactttacaggcccactggagaaagttatggcttctggttgtggttggaggttcatt
cctgagggagtggggacatttctacacttcttcacggccaatgacattggagaaactggcttcctaacccagcccacaccctcgc
acacacacatcacacatcatggctagaatggagagaaattcttcatatggggcacttgtacttcatgaaagaaaatcatatcaatc
ttgagtattttaacatcctattacagcagggtcactgataaactaagtgtccagagtgttttctaggatggtgtgtggtctccaaattaa
cattagtgaagcttactggaaggattgttactcctgggccaggccaggattttgaggagagatgtgtttgctgtcaccaaatccttga
cagactttggcagaagtgtgttaggcttactctggatagcttcagaggacaaaactagtattgacggaaggaaggtaaggagaa
gcagcttctaacccaggggaagagagagtttccaaactgagaaatcaaaaatggtactgattccttgtcagggtcagtgcttctcc
ccactgtgtgaattacaggggccatttgtccaagattccttagagcaatactgatttcatgtaattatttgaatgaaaggtgatttgttaa
atttatagtaaaatataatttgatttgtgtccctgtttgtcatgccaccccagaagaaaaattgtctttggttaggtcgaacataatggtttt
ttggtttgcaaaccatgagcgattcccatattaggtgggagttcagattcaaagggccctcttttttttttttttttttttgtagtagccagcct
aatgagtaggaagttgttctcactgtcattttatattgaatttcttttattttgagtatgaccatcttttcaaatgtatgagatagttatttccagt
tccacatactatctgtacatttcttttgcccgcttttagtttgggtctttggcctttttcttattgatttatagaagctcttttatacatagaaaatt
aatactttgtgactagttgcaaatattttcagttgctgaaatacacagtaggtgttccatgtaagagctgaacagctggttcctgattgc
tgtctccctcccttccagccaatagatttcagagtttgggcattacctattgagccaaagctgacaccacacaagcgcagagtatg
ggaacagagttctctgtctgattcctgtgagcttcctcatactaaatcaccaacagcaacctacttatcacagaatatgagaattgaa
caagtgttggcaaggatgtggagaaattggagctcttgttccagttgtcgatgggaatgtaaagtgatgtcgctgctatggaaaata
gtgtagcagttcctcagaaaattaaaaatagaatgaccacatgatctagcaattccccttctgggtatatacccaaaagaactgaa
agcagagtcttaaagagatattcatacagccttgttcataccagcattatgcacaatagccaaaaggtggaagcaactcaaatgt
ccatcaaaaatgaatggataaacaaaatgtagtatgtacatacagtggaatatcatttagtcttagaaagaaaggaaattcaaac
acatgctacaatgtggatggcccttgaatacattatactaagtgaaataagccagtcacaaaaagacaaatactgtatgagtttac
ttataccctaagcagtcaaattcatggaaacagaaggtggaatggtggttggcaagagctgagaggaggagagaaagaaga
gttattgtttaataggtatagaggcttagttttgcaagatgaaagagttctgaagatggatgtagtgatgactgtacaacaatgtgaat
gtatttcataccactgtacactcaaaaggtgaagatggcaaattttatgtgtattatgccacaactaataaagatttctaaaacttatg
agatctaatttcaccgtttcctattgctaaagatcacaaattagaaaacacgttggcaaaaggtacatgaaaataagcactcttgtgt
tgatcagagcataaacgtataatctcataaactaataaagatttctaaataacaaagatttctaaaacttatgagatgtaatttcacc
atttcctattgctaaagatcacaaattagaaaacatgttggcaaaaggtacatgaaaataagcactcttgtgttgatcagagcataa
acgtataatctcaggggagaacaatttgcaactattcttcaaccctttggtcaaacgattctgcttctaggaatatagcttactcccac
ctgtgtgatatggcatataatcaaggttttccattgcaacaaaagattggaaacaacgttaagtatccatcactagtggtctggaaat
atatatatattattgtcatccaatagaatacaatagactaatatgcaacttttagcatgaggatactcgttacatgctgatacagaata
atctccaaggtagtcatatgtgtgcaaaaccgtacatagtatgctaccatttgtgcttaaaaataaaaagaaaacagaatatgggt
caatgtttttgtttagttttgtctaaagtaactttaagtagaggcaagaaactggtaacatgtaacagtgatcacccctgttacctctgtg
gaagaaaactagacagctaagggacaaggctgggaggcagacttgctttccactatttatcacctttatctttcaaatttagtaccat
ctacatttagtaccatgatctattcaaaaatatttattaaaaaaagaaaaggtatagtctagaaggaaaaaaaacataacagaca
cttctagcccaatgtcctgcactgggtgctatgagagcagaggaaagaaacacatatggcttctagacaacaccgtctggggcat
acatttctgctattcgatcaagaatagttgtgcatcttttcctggaaagaattgatttgtttttatcaacagacctatgaatttagtggaca
gacctgtgaattaattcactggttaggttttcctttttacattggctgttaaaaagctataagccaaatttatgtccccctcagtgcaaatt
gggcagatttctagggcaagcatttagcactggccttgtccttggctctgtatcatattcctgtatttggtttgcttttccacctgtttctcatg
ttggtcatctttcctgtgtatggccataccatcctgaatgtgcctgatcgcatctaatgttggtcacctctccttattctttgcttccttataag
ccactaagcagcctttttggtgctagttagggtaagtgcgtgggtagtgaaggagggaggagggagaggaagaaagaagata
gaggttataaagcaaagcatatcctttttcttggcttcatcatgtagattaagtgaattgctctcaaagcgtggtccttaggccggcag
cattgtcatcaccttatgttgttaaacataaaaattcatgggtttcatcccaacttactaagccagactttctgtggttgaggcccagga
aactctccaggtgatttttactcacattcaagtttgagaaccacaggaaaacaaaaggaaggcagatttctaagcgtaaatgcaat
actaaccgattgcccccatcatgcctgttatgttggtcaagataaataatactagctactgcaataatcaatccctcaaattttattttttg
ccaatatcacaatccattgtagatcagttgtgggagaggtgtaaagagagctgctttattagtttattaagcaaaccagatctcttcca
ttgtgagactttgcgattttctaggcccttggacatttcctctggatcccctgctgctaagaaggcaggagagggaggaaagagaa
gagactttagcagccagatctggaagaaacatcttttctgcccacaattccattggctagaagccagtctcatggcctgtataactg
caggggaggctgggaaatgtgacctatcgatggagctaagagcaaaaggaaatggctttgatgaagccctggcattgtctctgc
acacccgagaacccaagtgaatcccaaactccacgtccaggtcatgttttggtgaacatcggttttcagtttccttttctaatcaagttt
tacctttttttttctcgactctagCACTATGGGACTGAGTAACATTCTCTTTGTGATGGCCTTCCTGCTC
TCTGgtaagaacctttcagctttgttaagtcctggaatcctactgtctcctgatgagtctgaccacagcaagcccaggcctgaga
cttggtgggttttactcactttctactgagcattgtacaagaccacatgcaaaaaagactttcctggagaagaaggaagtgttatgat
tgagagcagctgatggcaggcagctgggatggagctctcccccccgtgtgcttcttcctcctctgcagtctcacatcagtgagccta
gatgctcagagtagggtagcctggcccatcccatggggatgggggaaggctgctgcactgaggcccctgagacttgactcttttgt
tccacacatattctcttctggtcttctctgaccctgtttctgtctttctcaggctcctaggaaacaactgacagaattccaaaagtctccct
tcattcggagcactggctttcacgtccctgacttccctaccctctctcactcccttccctacagcccatgcacatacctcatggttgcca
cggcttcctgacaactatggatgttcagctaattgtgtcagctgatttatagtggagccaatgaagctgaagcttcagagccctccat
ttgcacaaccctttctaaatccccctcaagaccctgtgaagggccccctagcagtgtggtcacctgtcttatgctttggtaaaatttga
ataagtaagatattgtaaccacaataagttatgaccactgtctccttcctctgcaacttttccctccatgccattctcctgtctggtggtgt
tagcagtcaggggcattttgtatttgaattctacattctttttcttaactatccaccacctcccctcaaaattttaacagcatccagcctca
caaaactcagatcttccctgtttacagttccactttgagtttcagtttcttcatctataaacaggagttggctgcggtccctgccatgtatc
ctgtgactcagtgtctcgtagttactcctggcccaccccttcctgctgctccttgtctccacctgcaggcctgagagggaagccaccc
cactaagacagggaggtgaactgagcctgaagtttggctacagcacccacaggccaccagccatgagttcacctcctccagat
ggccacacaccaggcccttggccactgtccccatgtctgctgtggatgatgaggagtcagggaactacaaagagatggtccctc
agatccatgctggctgggataagccttttcagatttctgtttttctgcttagcaccttgagcttgtggagtccttgagtgcaaggtctgtag
atgtgccagctgatcactgacttaggtaacaacagcagcttccaacccccagggcccatgacctgctaccttagctcctggggat
gtgggaggtatgtgtgtgtcagagagcaaggcaagaagactctagagaacattatccagtaagattcccttctcatcccacttctta
tttatttattttatttattttattttttgagacagcatctttctctgtcacccaggctggagtacagtggcacagtcacagctcactgtggcctc
gattacctgggctcaagcaattctcccacctcagcctccccaagtgctagaattatatgcatgagccatcgcacatgacttattttattt
atttgataaatgcatatatacacacagtcatgaatcgtttaacaacaggggtacgttctgagaaacacattattaggcgattttgtcat
tgtataatcatcatagggtgtccttacacaaaactagatagcatagcctgctccatacttaggctacctggcacagcctattgctcct
aggctacaagcctgcacagcatgttactgtgctgaatactgtaggtgttgtaacacaatggtatgtatttttgtatctgaacatatctaa
gcatagaaaagatacagtaaaaatatggtgttataatcttatgggaccaccattgtatatgactgaaatgtggctgtgcaatacatg
acagtatatgcatatatatatatatcccttactttgtgcctggtactgttctaagtacctcataaatattaactcatttgagcctcacaata
actctctgctttaggtcttgttgttatttcccattttaagatgtggacactaaagcccagagagatgaagtaatttacccaagatcgaca
gagctactaagtggcagagcttggattcacacccagcaatgtagatttagcattcgttcacttgactcttctcctaactcttgtggtaaa
ccatgaataagtggtaagacttcttccatggggcctgaacagctttggtggataatatagcttctgcctcatccgtgttcatccagtgc
ctcctccccatcacctgcagctgacacctcagttgacccaagagcttgggcccaagcccttctcatcaaagtgaccagcccagct
ctcaagatctgggagagaaggaagaaaaatgccctggaaacacatttccagaaaacactaaactggaacaccatttcccacc
aaattttctgactccgcacactgaaagtgagaaagtaaagccgagacactctatgaaaactgagttcaggtgtcacttttgcccttg
atttgccattgacacttcttagaagtttcttagctcctgagaaaagagttaccaatattgaaagcaacaacctcaaatggtaaccgttt
aagttttatggtggtgagagaataagtgactatatttttggcagtacaattttaaagtggaatagaaagcccatgacatcagatcag
aaaataacattgccagtaattcacacacgatgaaaagcaacaaaaaatcagattctatttgaattctttcttctcagggcacacctc
tgcttactgggctggtgaacagtgacctagccacagggccggcttccaaagggagaaaggagatgcaattggcccacataatc
caccctcaaaatgtagagctgaataattcatttcatggcatagaaatagcaatacagtgaagcaattctgtttaacttttccctcccta
tattttgtgtcctctgtcatggaaatttgacacagtagtatttgctgcccctgctcttgaggataaaattggatgggagtttaagactgaa
acgggcacctgtggccttgcagaattaggttacagtttgtgccttgtatttacaaagcgaaaggaattcctagtgccacctgcagag
gcacttctaactttcaagctctgtttgccactgtcctggcacctccatcacacttttaggctggagccagagaggtttttgaaaaatca
gtagctcccacatcaggaggaagtatctttccagtttgagttttggtagctgctctctttttgtctgagggttctctgggtcctagggctttc
tcatttctcttgaacaacacctctagttaatttcatgtacctggagtggtagttggaatatttcttcactttaagattttttttttttttttttgagat
ggagtctcactctgttgcccaggctaaagtgcaatggcatgatcttggctcacggcaacccccgcctcccaggttcaagtgattctc
ttgcctcagcctcccaagtagctgggattacacctaccaccacaaaatacaaaaatacacaaataatttttgtatttttggtagagac
ggggtttcaccatgttggccatgctagtctcgaactcctgacctcaggtgatctgcccgcctcgacctcccaaagtgctgggattac
agacaggcatgagccactgcgcccggcccaccttaagatttatgtaagattggctcaaaagctcattcctgtggaaaggtccact
gttttcctcccaagatttttgcagatatctgcgtgggtggttacttttgactcccatttcctgctgttgttgatagccctcattaaaaccatca
cctggaggtgaatagacagtcgagacctatcattcccaaagaattgtcatggagcctaatagttctattggattcacccctttatgtta
agccaccatttcagtgtttttcaaaatagatatatgttatctagtagggagtatcttacccccaaattagttgattgtttcaggagggcttt
tagtgggttccagagaaaatgagcaatcagacaagttgatttagtggaagacagtcactgaataggatgtgtatagggttgtttgg
gagcaagagtgaaattggtatggaacagagaggctcccaaggcaagcagacattttttttggaagaagcaagtgtttgagagac
tgtggcttatttttcctttgtgagaggggagttttaataccatttccaaaatatgtaacctggtattttgtccccagaagtactgttgagattt
atggaagcaaaaaactctgtcacccaggctagaggagtgcagtggtgctatcaaagcttactgcagcctctaattcccaggctca
agagatgtttctgcctcagccacctgaatagctggcactataagtacatgccaccatgcctggctagttttttttgttgttgttttgttttgct
ttagagacggggtctcgctttgtgcccaggctggtcttgaactccttttaagtgattatctcttctcagcttcttaaagtcctgggattata
gagtaaatttatctataaattattgattttatgtcgatagacattgttctctatcattaataatgttaaaaataaataaaaaaacaaaaac
aagtaaatcaattaatgcttaccacaggccagtatttgatccaacactaactcaaatattcatttctttaatcctcacaacaaacctat
gaggtaggtaccattattgttcctgctttttgcaagaggaaactgagacacagggaagttaagtaatttgcctatggtaacacaggc
agtgagtagttgagctgagattgaactcacgctgtccagaatccatgctattagttataatagtgtactgccctatagctttctgtttcac
agctacatggcattactttgtatggatgtatcattatttgttaaaccatttaacttatttccagtgtattgttcttataaacaatgaatacctgt
gtacctctaattttgtgcacatgtatctttttgtagaatgaattcttaagaaattgagttgctaagtcaatgcttaagcccataattaattttc
ttacatattaccaactgtcctccaaaaaggttgtaccaatttagaattttaccagcagtaaattcagcagttaggacccattttcctaac
actctcgcggacactgggtattaccagtattttttttaatacgtgccaatcaaatgggcaaaaagaatggtttctcactgaggtttaaat
tgcatttccctagttattcttgagatttttcctttcctttcttcaacaattacttattgagtgcttcatatttgtaagggacaattgcaggtactg
gaaatgtcacagtgagaaaagtgacaaagcccctgctgtcatggagcttattctaatgggagatgtcaggtgctcagctgagctg
ggagagagagagctgagttgtcaggtgtcagaggagccaattatagcagcaaaacaaaaataaaatagttcagcttttaatctct
tactacgacggtataatcaagaggctaaaatgggaggaagggcagactctgcctgttccatttccccacatagagtgagtatacc
agtcgagggtcaggtaatcagtgcagacttagggggtcgccttaccattgaagaagccccaaatgaaaggctctagcagttttat
ggacctgggggtggaggaatccaagggtggggagaattcatgaggaaaatgaggtgagagggctaggagtggaaaagtac
aaagtactgagttagcgtggggaatagtgtctttagggctaggagtggaaaaaatactaggtactgagtcagagtggaaaacag
tgtcttcaaggcagggagtggaaaagtgctaggtactgagtccgagtggagaaaagtgtcttctctatgatgaggaggcttcagc
agaggtgcctgaagacctcaccccagagcctcagataaagagacctaagaatgagggtgcctgggctaagattgcaagtatgt
gaaaaagcatgactggcgggaggctgagatcttgattgcagcccccttcagagactgccatgcactgactgtgcaccaagtctg
ctgtagaaagggcaacttcctcagcaaggcttgtcagattaagcctctttaattgcctgtggtcaggtctgaaaaatcacacataga
tttttaatcagaacccagacatctcaggagagacagacaataaccaaacataccgtgtcatgtcatgtcatgataagtaccacaa
taaatataagtcagcatgagggacagaatgcccaggatgctatcttcaatagaatggttagagaaatctccctgggaggtagcat
ttaatgaaagacctacatgaagtgaaggagaagctatgagactgtctggaggaagaaccttctggacagagggaacaacatg
agaagaggacttgagacagagtgtgtgatcttttggaggaatgtcaagggaggcagtgtggctggggagagtaagcagggga
aagaggcctgataggtactggggacccaattacatgaggtcttgtaaggccaggggaaggactttggatgtagttctcagtgtga
ggggaagggatctggatatatttttcagtttggtggaaggcatcagaggcttctgaacaggaggattatgtgattggagctgtattttt
aagggatcattttggcttgagaaactagacccggggacaaggacggagcaggcagatgagttaggagacaattacattagtct
cctctacccttttcttaacatattggagttcagctctggctgtagtagttctagatctcctcagacacacttgtgtagagcctctgttgggt
attttgggtacacaaatgattcatcttggttatacagatgatttagatgattgtagacagaagagggttgtctggtcattcccagacag
gggagcattccttgagatagagtagaggaaggctgaaggggaggaagacagtacctgttgctatctagatagagacatccagc
aggaagttgaatacaggtatctgaaactctagtgaaagttataggctggcaataagcacctgggagttattagcttttacttgacagt
tgaatccgtggggctagaggagaaaaaccaggaaagtatggagaataagaagaccaagaacatgcactcaaggttaccaa
aattaaagagtgatttgagaaaattaacaaggaaatcagagattgggaaagaatagagcatttcaatgaggagagatgccaac
acttgcatttgacacagcggtcaaatgagttgagatctgaaaagagctcaagccttggccatggtgtgaagtcaccaacaaccttt
gtcagggagtttcagtagagaggtgggggtgggaggctgggaataaaggcagcaattgctgcttactctttcagggagtttgactc
caagggaaagagaaactaaaagcagtagcacaaggtttgtgtttgaagtaatggaggtgaaccaggtgaatagcctggaggc
cgagtgaagtgagacaggacactgcagatttggaatgtcaccagtccgcacaactgaataatttcctccagaactgctcaattgc
ccagttgtaagaacagatatgtagaccaaaagtagagtgtccccagggtaaattttatagagacaaaggggtgtgtttattgaagt
tgtggaaaggaataattacaaagacatactattgttgcattgtccaatataataaccactagccatatgtgactacttaaatttcaatt
aattaaaattaaataagattaaaaattcatcttctcagtcatactagctatgtatcaattgctcaatagccacaggggctggtggctat
catattgttcagcacagagacagagcatttccattatcactaagagttcttgtggaaaacactgcactacagggtctggataaagct
gaggtcttgattaagttgaacaacagttgtagaaggagtaagcaagagcaaaacctggatgaataggaggttgtggacggaga
ttagtatattgagattaagattctagggactgagctgctccaggtgaaaagtttcagggttatgtcataagaaggtggggggcagct
gctgaaatagtctgcgggtgtagacctgtggagttgacaagatcaaagaaatttgaggcaaggttgttagactcattcatgaagaa
gtcacccaaattgttagcaagaccttgcatctaatgccaaaatcctcatttagcaaggtggtagtgacttagtagctacaagcaatg
agaaagtcagacacctcaaaaggggaaggtgttgctcaaagtccccacaaagtgtgataaaacaaacagtagctggggctgg
agcaagtggcttcctttgggtgaagccagatttcactgaaataataacctcagggaaacagtcaatgaaggggttaaagatgtgg
gagagtttccttgtagtaagtaatggaatgaggctttcaaagggccaagtaaaactttggaggaagtttagtaaaagaaggaatttt
ttttagtacagataagcataggaacataaagaagagataattcttaaacatataagatatgcatttggggatagcagccagggaa
cactgaagtcccagtggggtcagagacttcataaggctagcaaattacagtttttgagtggcattccaacagtagagtgtattgctc
aggaagtccttaattatcctttgaaacaaattccttcagctgattacgaaggcatctagctggattcttgagcgacttgttcctgacatc
atagcaacccattgtaactagacttcgaccattcctcttacccaagtgctggggaagggagagattctcaatgcttacccacctatg
gaatcccagtaagtccagttgctaggtggcttgaggtctggggtcataaaatggaaggcctgaagtcatttggtgatcacagacctt
gagccaaactttccccatttagtcagagaaaggattagcagcatcccccatgcctggctctgtgtgagatcatggaagccagtggt
tggtgaggtgctatggagtataaattgcaaaatactttcagttccactcagaatggatttcaaagtgatttccaccccatggggagg
agagggagtctgaggagggatggatggaaaaaaaattttcatgtcattttctgtgatccactctggagacagaggcagagattctc
tacaacagctgctcaaactatagctcttgttaaaatggaggttctgaatcagtaagtcttgggtggggccagagattccgtgtttcag
accagcccacatgtgacgtgaatctcattggtccatacatcacactttcagttgctaggtgaagaagggagcactcgatgagtgg
aagagaaagccgttgtaatctttgggagaaggggcctgggtcagcggagttagactggtctgtgagtggacagaatggatggg
aaggaaagaagatactgtgaggctctacagaaaaaaaaaaaaaaaaaaatatatatatatatatatatatatatatgtaaatca
agaagacagaagcagctaaagacgaagtcatttccaggtccagaaggcacaactgacagctgagtaataacataacattgac
tgttaattggcagaatttttaactgtgtgtttggtttctccatcaggtcatctgtcctatattacatgacaatttagactaaaaccagtatttc
ctcagagacaatgctagaagcttttacagtagggggcactcttgcattacattaagagctcagcaaagaagatgcagaagcctc
aggtttgccttgtaaggtgattcataaacacactaaatcttccttaggtctccctttcactgtcagggtacgcatatagattttccttcctc
cctccaataccggtacgcatcctctacaggtggtgcattttatacctcaagtacttcacagggtcctagtgagtgtagtgaaataggc
agtgattcatatttgtgcaaactcccactgatgcctgctgtctgcttccctaagagttcaagaccaccaccaaccccttgattatgtgtt
ctcactgggccactctgtacacagtttagtttgacaagtgcatgtcactgttatctgtccttctattccctctttcaagagaaaccacatc
aatttaattactcccccacttagaactcttcaaatgaagctcctctcatctctctcatcaacccatctcctccctttcctcctcaatgtcaa
catgccttcacataaatcctgaatgatgaaattttatttagaacttacactaacttcctctccaaggtggcatctaacttcatattaagta
agaaacagccttcccactctccacccccgcacttctcacccaccactgcttactttttttttttttttttttttttttttgccaagtctcaagtaatt
ctgtaacctagaaaaggtcctacacaaaccccgtgatcattcacatttaagtagttgggtggcccacatccttcccacaaacccca
aagtgtcctcaaggactaaagcctttctctcaacccttccagcatgatgtctatggttgtaaaattgtccagggtcagtgcatactggg
agcagcaagtttgtggtgcctggggtttccccaatactcccaaagcacatcctcacctgcccatctatgattcattttcagcatttcact
catgtgccttaaatggtcattgaccaccacaatccgaaaacagccatcaaatttgcccagttctctttctgatctctgaaagagctta
gagaggtcactgaaaataaaggccttggttcactatcgaagtcatttctaaagcatttgacatccttggaagtgctggccatgggag
cagcagtcataggggaagttctgtaaagggagctatttgaatttcaaagatgttactcaacgtgattccccaactaatgaagtataa
taaaggggggctataatttattaccattatcagcaatcttttcaccatagcagaccaaggaatatgtggatgggaggggagggga
aagcttttggtgatggtgtagaagttatggaacctgtaacagctacagtgatgaaaactaaaattaaggttataggaaggtaactg
gtgggtgaatgggttgtctaactctactggtttttccctgtcttgcaatttaaattcacagaaccacagtactagaaagacccttggaa
catttagtcaaccacttcattaatcagatgaggaaactgaggctcataaagattgcagtttgtacaaggccacacatttagtcagcg
gtgaagcaaggacaaaggtcctaatctccagatgccaagcagatgtgcacagttccagagcttaatatcttattcttcagcatgatt
actgataagatagtatctgggtattgtataaagagaaatggaggttttttcccctttcctcttgtttctccctccctaatccttaaccttcttttt
tagGTGCTGCTCCTCTGAAGATTCAAGCTTATTTCAATGAGACTGCAGACCTGCCATGCC
AATTTGCAAACTCTCAAAACCAAAGCCTGAGTGAGCTAGTAGTATTTTGGCAGGACCAG
GAAAACTTGGTTCTGAATGAGGTATACTTAGGCAAAGAGAAATTTGACAGTGTTCATTCC
AAGTATATGGGCCGCACAAGTTTTGATTCGGACAGTTGGACCCTGAGACTTCACAATCT
TCAGATCAAGGACAAGGGCTTGTATCAATGTATCATCCATCACAAAAAGCCCACAGGAA
TGATTCGCATCCACCAGATGAATTCTGAACTGTCAGTGCTTGgtatgtggtcaatggtgtgtgttcaga
ttcttagccttctcagatgagactgcaaatgagttagaaaaacactggagggggacttgaggggcccaggggaaaaggggggt
ctatagagagaaggcagaggacagccacttctgggaagtgcatttgaagggagtgtagagtctgggagtagggaactgaaag
tcttttgtactttttatagtctgcttctgaaggatcagtaaaaatctgctttggggaaaaaatagagctaattgaacaaagataatatgg
ctaattacctatagtaaaaaccatggataatttggccatcacaaagtttatataaccataaaggcctcagatgtcttacattcattttttc
cttgggtccaagatttttcacctactaaatctttgcctggagctcctagcaaagcggacagctgacacatttgggttttcccttcagcct
cctctaggttgcttatgagttgtttgctgccacaaccatgagcctggtagacagaagggaaaaaaacccaacaaacataaccca
caaacttacaaaccagctcctctgcttcacgagaccttggaaggcctaaatgccactacagatttttttaaaactatcacacagtaa
aattatttttttttgttttgatatactgttctactgattgtatagatcttgtatagatttaggtaaccgccacaggacatagagcatttctatca
ccctaaaaatttccctcaggctgtcccttcatagagtcataccctgtctgcactcataacccttgttgggcatcctatagttttgtctttttg
acagtgtcacataagtgaagccacacagtatgtaaccttttaagcctggcttctttcgtttagcgcgccttcgagattcacccaagttg
ttgcacatatcgagcttgtccctttttattgctgagtagcattttattgtttatccattcaactcagtaaaagacattgggttgtttctggtttgg
ggctcttatgaataaggctgctgtaaacgttcatgtacaggtttttgtgtgaacataagttctcagttctctagaggaaatacccaggt
gtggtattactggatccaggttaatttttgatgaaacttgaaaaggcagatcaacacctattctaaaaccatagagtaaaacagaa
gcaaaagtaaaaatagaatggagagctgctccctttgaaccctgtgtgatttaaactaggctgcagggctttaggaatagttaacc
aagtgctaaatccgtgttttcaaaatgtggtcaggtaccattggaaatgttttaggtgggacacagataagcattttgaaaagccatg
ttgtatttgttttaatgtatattagaaaaactctaacttacgcaacatgtgatttcacagatcttgttaatgaagctaaacacggtctggc
aattcaccttctacaggccacatagactccaagaagactgctcaaatagtacactgatatagcaaaacttataaagatgacatgc
aaatgacagaccttttagtaagaatacactaaattataaattagtttgtagaacctgcaaactacctagtaactataaaagaacaa
gggattttttctgacagaaggcacatgacacaggtctagggactccatgccagtgatcctgaacagccagaaaagtgagaatgg
caaaggcaagagaaacactgtgtttattaagatcatgtatttttccctaaaatagctggatttggccttcttcttagagtatgttatgaag
acactttgatgctcatgccaaaaatcagtgttctgaatttcgaattccaaaatatccacccactcacttaccacaatcctgcttgggttt
ctgaaagatatgacgcagggcatctcagcaccatgaactctgtcagttcctggtgagactccagctcaattccttcctgctctcttagt
ctggggagctggaatgtgccccatgggacacctgggccctagagtcagaccacttctccttccaaagactctactccctggaaac
agtggcttcattgtaaatctttggtgactcaattacagccctcctgtcacttagagagcacccctttgatttggataagcaggaagtaa
gcatggctgcaaactctattgttgaaaaataaacatgaagtcattatgtggcactcaccttgggctgagggtcacattttagacacc
ctgaggctcccaggtgtgccccaatgagccccagatcaagtacccagttatttgctattccctcctagatacatctaaacttagattg
atttttttttatctctcttctgctttcagCTAACTTCAGTCAACCTGAAATAGTACCAATTTCTAATATAACAG
AAAATGTGTACATAAATTTGACCTGCTCATCTATACACGGTTACCCAGAACCTAAGAAGA
TGAGTGTTTTGCTAAGAACCAAGAATTCAACTATCGAGTATGATGGTgTTATGCAGAAAT
CTCAAGATAATGTCACAGAACTGTACGACGTTTCCATCAGCTTGTCTGTTTCATTCCCTG
ATGTTACGAGCAATATGACCATCTTCTGTATTCTGGAAACTGACAAGACGCGGCTTTTAT
CTTCACCTTTCTCTATAGgtaaagctgttttccaagactatttctttcagcaggtattatacacaaatgcttaaggcagatc
atccaatgtccccgacttgctaggaaacctccaactgggccattttatgacgctgttaggaaggacccagatggaggtctcctgctt
ctcctgagtgatgcagggtccaggaggctacgagcctatgttgcacttgaagaaatatgcttttagccctgaaactgactcagtctct
tggtttacctttggatggaggattctgaagttttgatttaaaaatacaggattcctccaggctagaattctttctttgattacaacacatac
atgcgcttgcacacacacacacacacacacacacacacaccatgcatacatgcagacatacaaatgatatttattgtgagtata
gaaccatttgggacattattggtcacaggagtgaaaacaaaaagatatgacaccccctctgcccttgaggaccttccaatagaat
cagaaccctgtaatgtgcacacatgaaaaactggatttttaaaaggttgaattggaatctaaattttattccatggaaatatctgacta
aatttaaaataaaagtgactggtaatgagatttatgggcattcagaggtaggcaagatccctgagggtcagggaatggttcctaa
aggaaggggtaccttgtaacatgtaaaataaattattggggttaataaatgtggtgaggaggggagggcattctggatgacaggtt
cccaaaactgtggtgacttccgtagctgaaaaaatttgagacagtatctgggctaagcaggtgagaggaccacagtggatcagc
tgtatctgacgtaagtgcaggaggtatgtcaaagaaagccttggaggcagaaatgcttgtgtgttcacaagtattcttcagggaca
agttcagtggaggaaaggattgaaactaagcagtagccactaataggagcctgacattttaaagtcctggctttacccaggagg
gcatgtgtctatatttgactcctcttttaagaagctgtaactgcaagattccctcctggaataaaggtggtctgcatctaccctgtcccat
cactgcctgtgctgaccttgacacccacatctgccttcttcttaccttgaccccttctccagcggtgatttcttggcttgccccctccagtg
acatccatccaactccttgctccataccctggctttgtcacctcctttctcccagtgtcttgttgttcagatataacttggtctgtgaacag
cccacggggccagtccccatgaaccaactttacaactgggccaatctcatctcctgctactgacttcttcctattcagacacttcagc
ctctgagaatccagtaaatggtggagccaactcgtcctgtcccagttgcttctcctgtatcctctcttggccagatagaagcctctcca
agctatgcctgaagttcagtacctccttcaatgtgtaattagtttgattggtggccacaagatggccatatatgacatgccccagggc
cctctgttacggctcccatagtctacaaattaacaggggcttgccaccactataacctcatcatggctcaccttcctgctgcttctcaa
ctactgttctgccaaacttcaacaggtacccccatcttcagaaatgtttcagctctagctgcctcaggaagatggggcttgcctctctg
ggtttcccttctatcgcttgatcagagataggttagaccctgagtcaaggggccttttttgcatgttaaaaggtagcagcctccacgtt
agtaagtataacccctaaccccctttactgggagtgccaaactggctcaagtggaatagactgggacagactcaaaagggatta
aatatggcctgcaatgccaacaacttcttaacatcccagaaacagggcatgtgtctacaaattatagctaagctaatagatcagct
ggtcctaattttcctgaaatttgggattagctaccagaactgttcccaaaaatgtctttaaagtgggcgactccgttctaagttttcccca
caaagcctgttttccaactccccagaaacttaggagttctcatgtaaggaagtagttcctgaaggcgtgaaggttcctcaaggcat
gaagaaacatcaaaggtttttcagtagatgagatatgctgaaagccatgcagaggaaacctgctgtgacctcagtaggaaaaa
actaaacaaacaagcaaatgaaaactagaggtaggggcctgtggaagctgttccatttgtccaagtgagaggtgtctggagatt
atagtggacagaagaatcatcacgagaggaacttcagggcctgggaactgactgcagaggggggcaggatagcaggcacg
gcacaaatgactgcacgtgcagagcctcagcacagacacctcacccagattccagaatcacgggccaggctgaccctcttcttc
ctgatcatggtcggtgttatccccacctccatgaaggcatggcagctcagtccaggcatttggccagaggcatgggctcgattctta
ggtcgctgctgaggccctgagcctgggactttctatggcctcctattgtggatttcaggcttctctggccttagagccctggggagag
gctggcaggtaaataaagagaagagcagctagcagaaaccttttgtaaatgactctcctggctgattgaaaatttgtggtcatttgt
agAGCTTGAGGACCCTCAGCCTCCCCCAGACCACATTCCTTGGATTACAGCTGTACTTC
CAACAGTTATTATATGTGTGATGGTTTTCTGTCTAATTCTATGGAAATGGAAGAAGAAGA
AGCGGCCTCGCAACTCTTATAAATGTGgtgagtgagtccttgtcctccccacagactgtcactttgcacctacttc
ccaatcggctggctgccttccggagcttgttggctgagcctagactggcaaaaagtcaggaagttgttgggaaaaaaggttttccc
ttggagttttgagcctatacagactggcagtagcagataatgctgctcttggacttcaaagaaaggcgacatttctaacctctggttta
caaatgtacttctggtttccagggaaaactgattattacttgctttatctacctcacttcatgaggttactgtgacatatacataaagtaa
aatggtgaaaccactcctaaatgttaaagattgtggacctggtggtgtttaagcagggatatttgctaaatgaccacaagaatcag
cttctcgtctctaaaaaaatctaggtttcttatgaaataagttagatgaattattgcccattgacttataacaaacaatattaactttaact
aatttctaagtaatacatatccattatcatatataccaaaaataaaataatctataactccactaataagaaaaaatgattacacaa
atatttttggtgcctatctttaagatttttctgtgtatcaatctatgttgttttccataattaggattatcataagggttatttttcacaatttggata
atatatgtactgtgttctaattttgttatactaaatgtagcaagacaattttcaatgtcataaatatcattctacagcatcatttttaatggct
gcaagatattcccttttgtggatacaccataatttatttatttaaccaacctcattttttggacacttgagttagtccaatagttttgttattata
aacaccctccccactgacttctgttataaaaatgtttcatggggacaaagtggtccctaactttataataatgccatgcctttttgtagttt
ggtctggttctaagctaagattggactttatctcagtaattgcctccagtagtaattagtttgattggtgctaataattaaggtaaccttct
aactcacttatggtagaaagcacaagatgagtattgcctctggccagcatcttgtttttcagtatactgattttaaaatctaactagaaa
atagatggatgacattagcagtcattcaatgcatcctgctgtactttaaaaataagaaattggggagcaacgatcgaatttaaataa
attaacacaaagcatgtggcagagccattcaaactgccaatgtatggagtgtgctgcgagatttctatgatataaaagtataaaatt
cctagcacagatgtaaagacatatcatgcttgtccaggctttgacttttcaaggtgagagttttgagcttcactttctttcaacctcattgc
catttaaaattagtcaaatatgaagaagtgacttacatcttgggaataagctgtttgctagatttttcttcacattagaatgatcagctta
caaatgaaacaaagaagggttggagaaaaagattaaggatgtttcttcctccatgaggcaatcagaaaaaaatcaggagacta
gataggggagataaagaggatatgtgtgttcacatgagagaagttagaaggtggttaaataagctctgtaggtacagatgagat
ggtcagattgggctgagtggcacatacatgacccctaagaatgtaatgaagaatattggtaagaaaaagttatttattcagacagt
catccatgccactgagtttgatcaaagagagaagccttgctatcactgtagggagggaggtgcaacaggtataactatgccattat
agatatgatatatttgtaaatttggattctgtaacttcagcaatatctgccattgctttgtgggtactcctggcattggctatgtgataggta
aaataatgccccccacaagacgtccacctcctatactccagaacctgtaatatgttatcttacatggcaaaaggaacttcacatag
gtgattaaggcaccaagcttgagatggtgagattaacctggattatccaggtgggcccaatgtaatcacatgagtcagagaacctt
tcctagctgggatggagaaatgaactggaagaaggagagatctgaaacttgagaagctcaacccagcatttctagctttgaaga
tggaaggaggaagccatgagccaaggaatgtaagtagcttctagaagctggaagtggctctcagttgacagccagccattaag
gaaattaggatctcagttctgcaactataaggagctgaattctgccaagagaccaatgtggaaacagcagatccctccacagag
acacaagcttactgataactggtaggaatttctccaaaagtggagcttcctcctactccagtgttaatccctttctcagaggagacgg
tcctcaaactaactaacttggcaccaaaagtcctatccagtgttttctcattatagtttttctatgcctcaactgtatatatttacccagttta
ggctgtttaaatgaataaaaaggaaatgccatagttattctagccagtttccaatctctcttctctttttttgttttgtcaaatagggcagat
aaggcatgagaatttataactatgaattactgtcttttcccaaacagaaatcaccctatcagcttacccattgggagaaaaactaaa
atagctccccctgaaattttacttcctcatttgggtcttgtgtgactgaaatctgtatacaatgccctagcaacaacggtttttacagcttg
cctccctagaacaaacctaggagtctcagctgtttcaggaatgatttcttaaaggtaaagtgcctttttcaaaagaaattattattattttt
ttttaattttttttttgtgtgtgtgtgagacagagcctcactctgtcaccaggctggagtgcagtggcacgatctcagcacactgcaacct
ctgcctcccaggttcaagcgattctcctgcctcagcctcccaagtagctgggactacaggcacgtgccaccaagcccaggtaattt
ttgtattttcagtagagatgggttttcaccatgttggccaggatggtctcgatctcttgacctcgtgatccgtttttaaccaacatttaaac
agaaatattcacaggcttaaagactgaaagttagtgatatcatcacatttccccttcaaaatgctgaatttgtaagcaaatttaaaag
tttagaatctaccttttaattgtctgctttcatttttttgacagtggctttttttgatatggtgactattttgtcatgggtataaaaggataattcatt
ttgtgttaatctgaagacatctgaaatactgtattcaactataagtacctttttttacatttataagattctttttcaaaatttttatttgaatagtt
ttttgggaactactgaactaaactaggtggtttttggttacatggataagttatttagtggtgatttctgagactttggtgccacctgtcact
cgagcagtgtacactgcaccagtgtgtagtcttttatctctcacccctcccactctttcctctgagtccccaaagtccattatattattctt
atgtctttgcatcctcatagtttagctcccacttatcagtgaaaacatacaatatttgtttctccattcttgagttacttcacttagaataatg
gtctctggttccatcaaagttgctgcaaatgccattattttgtttctttttatggctgagtaatattccatgagggatatttaccacattttcctt
atccactcatgggttgatggacatttaggttggttccttatttttggaattgcaaattgtgctgctataaacatgcgtgtgcatgtgtcttttt
catataatgaattattttcctttgggtatatacccagtagtaggattgctgaattaaatagtagagttctacttttagttctttaaggaatctc
catactgttttccatagtgtttgtactagtttacattcccaccagcagtgtaaacatgttcccttttcaccacatccatgccaacatctatta
ttttttgattttttaataatggccattcttgcaggagtaaggtggtatctcatggtggttttaatttgcatttccctgatagttagtgatattgaa
ctttttttcatgtttgttggccatttgtatattttcttttcagaattgtctattcatgtccttataaacaccattatttttaagaagaaactttacaa
aaatagaacataaccagatttataaagcatctgggaactcagtcaattaagaaatagctcaagtaactgatgatgcttcacctga
aagaaggcctggagagaacagagatactgtcttcaaatatctgaagagctaccatgggatgcaaagattgagcttgatggtatg
actctgaagggcatctctatgaatgaaggttatgagagggtataaggaattaagagagacttttctaacaattaaaaggtcttttag
gccaggggtggtggctcacacctgtaatcccagcacttttggaggctgaggcaggcagatcaccttagatcaggagttcgagac
ccgcctggccaacatggtgaaaccccatttctactaaacatacaaaaattagctgggtgtggtggcaggcacctgtaatcccagc
tacttgggaggctgagagaggagaatcgcttgaacctgggaggcagaggttgcagtgagccaagatcacaccactgcactcc
agcctgggtgacagaagatcaagattccgtcttaaaaaatataaataaataaataaataaataaatagtctttaaaattgtataga
agaagtagacttctgcttcctccaacaaaggattaactgctataggaattgccctctttccataaacaactagaaagcagacaaa
atatatgaaacaactgttttcagagatcggatgacagacagcagaaaactgtagtccctgagtgaaggaaagaaaaaatgaga
taagccctatgattgctctagtttgctgcctggagccagtgtccaggcccctctgaaggcaggggagccctgatactgaactagga
aaagacattgcaagaaaagaaaactacaaacatctctcgtgaaatgcttaacaaaattagcaactaaaatctagcaatatgtta
aaagtataatacatcatgatcaagtggggtttattcaagaaacacaggtaagctcaacattcaaaaatcaggcaataacctttact
acataaataaactaaaaagaaaaaaacatatgatcatgtcaatggatacaggaaaaacttttgacaaaattaatacccattcata
gttttaaatggaaagaaaagctctcataaaaataggaatacaagatgacttcctcaacctgacaaaggacatctaccaaaaattc
ttctgttagcataatatttcatgatagaagactgattgcttttaccttaagatggcgaatgtggggaggatgtctactctctctacttttgtt
ccacattgtactggaggtcatagccagagaaacaagactagaaaaagaaataaaagacatacagattggaaaggaagtaaa
actgtcttttttcacagataatgatcatgcttgtagaaaatcctgaggaatctatcaaaaacctattaaaactgataagtgagtgtagc
aaagacacaggatacaaagtcaatacacaaaatcaattatttctatatactaacaaaagcaattgtacattgaaaaaaattaata
gcatttataatagcatcaaataatattaaaaacttggaaataaatttaacaaaacaagtacaaggtctatatactgaaaactataca
atattactactggagaaattaaagtaaaccaaaataaatggagacataggccatgtttatgaatcagaagactagatgttaagat
aaccattctctccaagttgatctatggattaaatgtaatcacaatcaaaatcctggtaagctctctaatagatactaaaaatcttactc
gaaaagttatagggaaatgcaaagaatctacaattgccaaaacaattctgaaaaataagaacaaaggttaaaaatacaaaatt
agccaggcatggtggcgcatgcctgtaatcccagctactctggaggctgaggcaggagaattgcttgaacccgggaggcaga
ggttgctgtgagctgagatcgtgccattgcactccagcctgggcaacaagagtgaaactccctctcaaaaaaaaaaaaaaaaa
aaaaaaaaaagaacaaaggtggacttaacctacctaatttcaatatttactatatatagtaattaatacagtgtgatattggtaaaa
ggacagacatatcagtcaatggaacaaaatagagagtcaaaaatagattcacactgttgacaaagctaccaaggtaattccat
gcagaaaggatagtattttcaacaaatagtgttgggacaattagatatccacatggaaaaagtatgaacctagacacacacaaa
gtaacttatatattaagaattaaaatgaaaggacttccaaaagaaaacagaggagaaaatctttgtaaccttaagttaggcaagt
cttcttagataggacacagaaagcaaaaaccatatcataaaaagataaaatggatgtcatcaatatggaaaacttttgttctttgac
tttgtttaaaaaacgaaaagtcaaaccacagacagggagaaaacgtttgcaaaatatatatctgataaggacttgtatccagtata
taattacatattgctactcattagtaagaagacaatccatttaataaaaggcaagaagaagagacttgaacagatacataacaga
agaagatatacagatggccgatgagcacagtcacaacatcattagtcatcagggaagtacaaattaaaacgataatgagatac
cactgcacaccctctagaatggctaaaattaaaaggtctgataaacatcaagtgttggagaggatatgaagcaactgaaactctc
atatactgctatacaacccagaaatcctagacatttaccaaacagaaattttaaaaaatttaaaaatatataaagactcatacaca
aatgttcatagcagcttgcttcataataccaaacctggcattctaaattttcatcagttggcggtggtatatttatacaatgaaatactgc
aaagctatagaaaggaatggactactaataatacacaagaacatagataaatttcaaaagcattatgctaagtgaaacaatcca
ggcacaagaagaatacacattatacaatttcatgtatatgaaatttgagaaaaagcaaaactattttaagtagattcatggttatcca
tgggatgggggaaaggaatcagctgaaaagcgaactattttggcttataaaaatgttctcgatcttgattgtggtggtggttacgtga
ctatatatattcgttaaaatcaccaaactctaaactgaaaatgattgggttttattatttattaattatacctccataaagctgattgtttttat
cttttatttttattttatttcaatagtttttggggaacagatggttttcggttacatggatgagttctttagtggtgatttctgagattttgatgcac
ctgtcacccgagcaatgtccactgtacccaatgtgtagtcttttatccttcatccacctctctctcactcttccccccaagtacccaagtc
cattatatcattcttatgactttgtggcctcataaaagctgattgtttttaaatacacacatacacacataaaagagaacttccagtgac
aggaagtgttcaagaatgctctatttagtaaagacagaatcacaaaaccatcagaggtattgttgagtggattcttgtggtctataaa
tacctccatggacacccaggttagcaacctgttggagtttacgtgggacaatagcatcatcacaacagtcagcctagagaaattta
catcccaagttgtgtcagtagcaagtccctatcaatagcaactcaggctttgtgaggtctagctggctagaaatttcccacttggcctt
gcccatgcaacattgtgtaatattcttagcaccatctggctagccgatttaggcatcaacatcttcaagacttcttctcctcctccttata
aaccttgctttcagaaaaggattagaaactcttccaatcacaaaatgattgctaaaactaaatatattacccctcccaatggtatttttt
ggttagccaggatagagatataagtgaaaaatctatttccagtgttagaatttaaggcacagtgagaaagggaaggcatatacttt
ttgaatgcaagaaacttcttcccaatccccctgaaattgcatcatttgagtaactatctcttccatatataaagtcacacaatttctctct
cagtcccagaactttgaagccttttcaaactttccttcttttggtatctaggaggaatacatttttgaagattgttcttggtgtctttcagGA
ACCAACACAATGGAGAGGGAAGAGAGTGAACAGACCAAGAAAAGgtaaatcctgaccctgagac
attgatgagagagaggtataatccccagagtgcctgttacttgaataggcttatgcctaacatatgttgagacctcagcaaacctga
actaatggagagggagaggaaaataaaactagttaagaactggaagaaaataacctgataatggatgacagggtatccaatg
cacaatgcccagaaagcatgacaagctctgtcatggtcaagtaaaagtcaataccaaagacttcagaggtggtgaacatgggc
ttcatcttatctgccacagtaaccccagtacctggcacagtgcctagattagtgggcatcctacatgtgtggaatgaataaatgaag
aagtggggaatgataacatgtttgcttcagcctgagcatcttagtatttgctatggccctgtttagatgttcttctgccacttctttacctca
ttcttcagatcttgcctcaagcagcactttcttaaaaaccctttcccaaactagaaaatgtcaacttgttacagtgtcatgtggatccctt
ggctttttcttaataacaccagattatgcttacatatttgtgtaattatcttattaaactctataaactagacttaactaaatcctatgaaga
gcagagaccataccagttaagctcatcattgtgctgctagcacttagcatggtgcctggcatatagcaggttctcaataaatgttga
aagaatgattgatgcatgatgaatacataaaagttcgtggtgatcagtcctttcacaacgtgaagctatcagatagtctgtacctcta
tccctcctgagaaattaagctctcaggaatatcaaggctctgactgcatacccataggatcaaagcaaccctcagtcacaagcct
ggtttcagagatagggtcataacccccagggtgcagagacaaccgagagtacccagcactaatccagatataccagccactgt
gattctagcaacaaaactaataattccgggcacccttggacaatgagaaagggtgctgaaatcctgcctaccctgtcacactcag
tttcagaaatggtctggaagagcctgcagagggcaggcagcagagaaccggcagagggcatgggaagggccaggcagaa
ataaagggtagctcttgaagcatagatgacagtgtagaccgtggttcttttctcttgctttctccacctttctcttcaatagtttgtttctcctc
attgctgttccaatggcaacctctattctgccctatcattgaaatctagaaaaagaaagtagctcaaatgtgaaatatcacctaatctt
ttcttctatttctccagAGAAAAAATCCATATACCTGAAAGATCTGATGAAGCCCAGCGTGTTTTTA
AAAGTTCGAAGACATCTTCATGCGACAAAAGTGATACATGTTTTTAATTAAAGAGTAAAG
CCCATACAAGTATTCATTTTTTCTACCCTTTCCTTTGTAAGTTCCTGGGCAACCTTTTTGA
TTTCTTCCAGAAGGCAAAAAGACATTACCATGAGTAATAAGGGGGCTCCAGGACTCCCT
CTAAGTGGAATAGCCTCCCTGTAACTCCAGCTCTGCTCCGTATGCCAAGAGGAGACTTT
AATTCTCTTACTGCTTCTTTTCACTTCAGAGCACACTTATGGGCCAAGCCCAGCTTAATG
GCTCATGACCTGGAAATAAAATTTAGGACCAATAcctcctccagatcagattcttctcttaatttcatagattgt
gttttttttttaaatagacctctcaatttctggaaaactgccttttatctgcccagaat
CD83 genomic sequence

SEQ ID NO: 3

ttagataggcagaaatttaaaaagatctggctgggcacgtggctcacacctgtaatcccagtaccttgggaggccaaggtagga
ggatctcttgagcccaggaatttgagaccagcctgagcaacatagtgagaccctatctttaaagaaaaaaatctgatcatgctaa
gacctgctgaggggagtgtaaatgggcatgtgcattttggataataagacggcaatatttaacaatgcagtgtaattactgagctag
agtgttggaagactttcagctcccctgcaacattgtttataatcaggaaaaactgaaaagaagcataaatggctaggtatgagatc
tggcagaggacacatagtgggtctcaaaagaccatcctggctaacacggtgaaaccccgtctaaaaatacacacacaaaaaa
attagtcgggcgtggtggcgggctcctgtagtcccagctactcgggaggctgaggcaggagaatggcgtgaacccgggaggc
ggagcttgcagtaagctcagatcacggccactgcactccagcctgggagacagatcgagactccgtctcaaaaaaaaaaaaa
aaaaaaaaaaaagagggtctcgaaaatgttagtactgttttatttctcaagaataaattgtatacagatgtgttcaattccatattttct
atacttattttgtatgcttaacattttcacaattaaaaaattaatttggtgaggctgctggagaaaaggtactcacacaagctggcggg
actgtcaattgatataactacttccaagagcagattagaactggtggtatagtgatgccactctttttaacctcttggatggacaaaga
tagaaaggttggataacagtttgtgttggcaaacaggcactctctttgcagatgggaatatagattgaagacacctccttgcaggta
attttttggcaatatttgacaaaattggaaactccccttcacctagcacaatttccttgaggtatttattctaagaaaataagcaattttag
agcaaagatttatctacactgaagtttcccatagcaatcacagtattgtttctaatattagtaatacaaaaagaaacaacctgtatgtc
taacactaatcgattctaatttatggtgcaactgaacaatggaccaaaatgatgctgttggaagtttttaatgatgtggaaccgcttgc
aaattattaagctaaaagaaagtaggttacaagatagcaggaagaataaaccattaaaaataccaatctgtgcactgacaaatg
ttataaatattttacgttatgttatgttataaacattttataatataaaaaaatgttaactgaagttacttcctggatgaattacaggtgattt
cattgtcttctagaattttcttttccaaaaatgttgtgtatgcgtgtaattattattttaataggagacactctcctttggtgatataatttaaac
aggacggtactgactgataacctcccggggaaggcagggagccaagtactacagacttgtatgtttccatggaaatctaacgcg
cctttgattatcacagattctggagaagagtgaggacttgggttcaccagtgcgttcccaaggacaggctgggcttctgaggaagtt
gcccaccctctcggaatctggtttggcctccgtaaaatgggcagatcccgctcggatggcccggttcccggcttccttttgcgggtc
aacggcagcgtcacgcgcgcgagcgcggtctgcaaagcccccagcgctgggcgtcacgcggggattgctgtcgccgctgcc
agccgcagcagcgacgcgaactcggggcgcccggcccgggcgcgcgggggcggggacgcgcacgcggcgagggcggc
gggtgcgacgggggcggggacgggggcggggacgggggcgaagggggcggggacgggggcgccccggcctaagcgg
gactaggagggcgcgccacccGCTTCCGCTGCCCGCCGGGGAATCCCCCGGGCTGGCGCGCA
GGGAAGTTCCCGAACGCGCGGGCATAAAAGGGCAGCCGGCGCCCGCGCGCCACAGC
TCTGCAGCTCGTGGCAGCGGCGCAGCGCTCCAGCCATGTCGCGCGGCCTCCAGCTTC
TGCTCCTGAGCTGCGgtagggctcgcgagcgcctgtctcgcctgtcgccccccgcccctccacgacaccccctcccgt
cggtcgcttgctcacgacgcgctctctctttcttgtagCCTACAGCCTGGCTCCCGCGACGCCGGAGGTGA
AGGTGGCTTGCTCCGAAGATGTGGACTTGCCCTGCACCGCCCCCTGGGATCCGCAGG
TTCCCTACACGGTCTCCTGGGTCAAGgtaggtgctgcgatacccacgggctggggtttggtgggctcatttgaa
gacagcaggaaccatctcccctaggctggcgaccctctgtggctgccaggtgggggcgaggggcgtctcccgcagctgaactt
ggagtacccagcctcccgtcgcgcctcccccaccccatccgcatccaggtacagggccgaattaggttttgctctccgcagacct
caatccccttcctgtcactgaaggtggcctgagatgaatgatccacttaagatgttttggaagggcagagactctcatttggattaatt
ctggaggccacctgtggttgtgggccagcaggtcaggaagaaagcaacagggacctagatttgggcattggacagggggaat
gtctccagacttctgatttcttgtgttttgtgactgtgatgcccatgatacatgggagggggagggggcaatttgaaaggaaaggcta
agacacagaagtgacttaggccatttcatccatggtagttatcagtggtcatctcctttgtgggatacccttggcttcctcccctagccc
tcctcctccttcctctggcagccttgagagcatcaggtggatgcatgagccggagcccgcatgtgtaagaacaggccttgctgctc
ctactgtaagtggactgagtgacaaggaggctttttcaaggtttcctcttgactgaaacattctcagattctaagatggcaatgatggt
gtcattccaaagccaagcagctactgtttgatatcactggtccttctttaagtcaggccactgctaccacagcacctccattttaaccc
aaatgaatatgatattacaaccttactctgtagctctcactgatttgctgtcttaccacgggggcaaatctctgcacttgtagctttcccc
aaaatgcagggcgttcttctgcccaccataaaagatactataagaaactgtacgtctttggccacttaacagtacaaggcatcatt
gcggtgatctctttgtgtgtgtgtctcctaactggatggtcagttccctggggggcagtggctgtatccatacttctgtgtattcttcacgg
cacctaatttttgccctataaattgcaaaggtgctctgtgaattcagcccagcacttcatgagttatgcatgacggggatggtgctgct
gcctcagagcattgtattgtgtataaaagtaaggtgttaaatattcctacttcattggtaccttacttactgtgggatcagagaacaca
acaattccgaaattgttctcatagtcaaaacaatagtatttttaaaaatattgtaaaaacaatttttgaatgctcaccacgtgccaagct
ccaaggtaaatatttacatacattatccatttccatccatcggaagaatggacttagggattagtactgttactattcctactttacaggt
gaggaaactgagccttagggagggaaataacttgtccacttttgcacagctagctaaatggtggagttgggatttgaacgaagca
gtctgattccaaatcctgagttgttagaggtctatcttgatctctgttttctcccttaataacttaagataaagaaaatcaaagtgcccct
gggctaaccaggcagggacttagttatctcaaagaacggggaaaaacatgaaaccactatcccttccagagagtaactatttaa
taaagaaaacattattaatacccccaggggagtaattaaaaagtactcatgaaacaagtagatgaaatttcaggctgtgaagttc
aaacagttctggagtgaaagcttcttgcacagggtcatttggaatggtccactaaaccatagcaattaaccttggacttctccttgga
tgtcagctggtgacgtaactcggtaacgcatgagcttgtttattggacagaattcttgcgagatttacccccaaggtctttgaaagctc
tgtcaagaaaaaaagggacagcagtctctaggcgttctttttttcctgttgatccatggaatagtgccaatgaaaagtcataccgtag
ttattttttgagaagtaaatggtgattgagattcgtgggtaggagagttatgctataccaataaacgaatcaggtgcctcgaaagtga
catatattgttcctttaagcattttttttaaaacagctctcagcatgttctgtagatacttattattttccagcccaataattatactttttcattg
attatgcttata
caacaaaaatggatagagtgttctggagacaaggccagtggtgaaatgccaaaatacttcattttacagaatgttaagcatctggt
catttttctataagtttcttgtaaaatgtttcatcaaagtggaggggtagccacaaagggaggaatttcattttggtaaccagaaccag
cttatcccatcctactcacttcatcatcactaccctggctttgtaaaacctgttttgccagcttaggagggggcttcatactgggcaagg
aaagcagagtcccttgcagtgggttttcaccatccaccagattgaagcacattctgcaggctgtctgcatatcataagtatggttata
atgactcacaatttaaaattctattcaccactcaatcctccggcaccatgtagcatcttgcctttgtccatttggcactgatacttgtaatt
aacaaaaggacccatgtaaaccatgtgttttttatcatatgcctttgaccagaaaactcaaaacagacagcatccaatctgtttgca
acattagggttgggaaggaagagtgttcattctgttctctctgtttcaaagatgcagtgagatgggctagaggggacttaatagaca
catgtgcaagaggctaaaggtgaagccaaaagtggacagagatatcccaattcctgttggcccagctcttctcttctatggaccat
gtcctcttaactgggatccaacaaagggtcctcttctcatcccttcctcccttatactttttaaggcataatgggtgattgagaagaaat
agaaaagttaatacattatattcattaggatagtagctcaatttagctttatgtttattttttgagacagagtgtcaccctgtttcccaagct
ggagtacagtggcatgaagatggctcactgcagcctcgacttcctgggctcgagtaatcctcccacctcagcctcccaagtagct
gagactacaagggcgtaccaccacacctggctaatttttatgtttttaattttttgtagggacaagatttcaatacattgcccaggctgg
tctccaactcctgagctcaagccatcctcccacttcagcctcccaaagtgctaggattacaggcatgagccaatcgatttatctttta
aagttgtaatagactgggtgtggtggctgaggcttatgcctgtaatcccagcattttgggaggcgaagatgggaggatcacttgag
cccaggagtttgaggccagcctgggcaatgcagtgagacctgtctctaccaaaaaaaaaaaaaaaaaaaaaaaaagttgta
atagatgtggttctttgaggaggtattttgagaaaatatgcaaatagactttgatccatgacttttcttccactggccatgacctgtgatt
aaattccagcataaaagggcatagcacaatatcatgtctgtgaggagtaaagccatgcattaaagggctgcatgtggacttcatg
aaaagcgtcgctgtgtctacactctctttaatgtaggtttggagagagaggatgactttggttggagtactttgggcctggttgataatc
actaaagatagtaatgagtgatcatttatcccagagttgcaatgccttcttgtatcatgctaggagccctgacagcctatgggtgatg
caaaacgaaagaggatatatggtgtcatctctgggtgatgctgcgggggtgaggagagtgaagcatcacaagacaagtgccct
tttcagatgatttccaaaggaagggagaaaagggaagtaagagtgtgacttcatataaaagtctactataaatagactttataatat
tgagaagagccccagctggggcagatcatgggccatccatggagtgttctgcttctgacattaacactaaggaaactgttggaga
gcaggttaatggcttgcgtgaggccacttcaaaagttcaaggctgtcttccgtgtatgttgctaaacttctttttggtggagttatgttttct
gtctctaccatcttgtgtgataatgagctacaaaaccagggatactgaggagagcagagtgccttaggagggcctagagttgata
agcggttggggcagatgtaatctgtacagccagagaccttcatagcccatggaaggagccagtactgaacacttactgtgcttcct
tgattccagaatgattctgttgtaaggtggatttaagaacatgttttaggacaaaaaggaaacatttctacattaaatgtagaaccatt
gaattatgaaaacaatgtatgttagaattaaaaaaaaaaaatcgtactgtccccattggcacctatagtacttgacctggttgaatc
acttttatgggctcctccctaggtcaaaccatgaaagatgtaaagttgcttttcagatgtctctcatatttacactttcattgtttagtagat
acttctaagtcccaaatgtgtgccccatcctgggcctggcattggccatctcaggatcaatgtagaacttttgccagaggaccatctt
gagcaaaggcctgggaatccactaagactttttgggaaccattgaggtaaccagtgatgtagaagggagacttaaacagcaga
tatggctgagagataacattagaaagtaggctagagacagattgtgaggggccttgaatgcccagcaacaatgacttgaccttta
tccttttggcagtaaggagccattgaaggattttttgtttgtttgtttgtttttgttttttttttttttgagacagagttttgctcttgtcgcccaggct
ggaatactgtggtgtgatctcagctcactgcaaccccctcttccaaggttcaagcgattcccctgccttagcctcctgagtagctggg
attacaggtgcccaccaccatgcccggctacttttttgtatttttagtagagacagggtttcaccatgttggccaggctggtctcgagct
cctgacctcaggtgatccacctgcctcagcctcccaaagtgctgggattacaggcgtaagccaccacgcccggcctcactgaag
gattttaagcaaagacaatggcataatgcaaaatatgcctaaagcaaagcatatttctcctggtgttggatagaatatgattcatctt
agaagatgagtctcagagggagacttcattcttttccttcttttcctcttggtcaccagtcctgtccatgtagttctgcggaggagtgggc
aaggaagaatgaggccgcctctgagtggctatagaagaagtctcatctagatgagaatggtggatcactgagatttttggacaat
agtggaacagagcacaagttgccaaaatcttttagcttgataatggggagggaggaagaaagcagctgagagttaaattgaaa
aaaaaaaaaaaaaaagctaaacaaaaaaaccaacttgttttccattaataaaagggggaacctgagtcacatgaggactgga
ttgtcttagctacgtacttggcaatgtcactacacaaagaagaggaagtttggagaaggtctcagtgacataagggaaagttttatg
tagggcaagactaaaagcagattgattacctaaaaaaagtttcctccctctaaagatgtttccgtaatcccttcctggctactcctgg
aataaccctaaattttgtatcaacaatcattagctcaaaatagagctgggcagaaaatacttccctaagattcttttatactcataagc
atgtttttgtttttcattttgttttgttttgcactgaggtgtatttgggtaaaatttccgtgtgtgtcatgtgggactagtacagacttgggagcc
caaggcttgttaatatcacttgatgctttcttggaggaccagtctactgcatatcccaaattgggacaatttggagaagtgttccagttc
ttagcttccagtggttgccagcagtcctcggggttaccgattagaatcggtattaccgatagaattgaggttaccgattctagaagag
ctggtagctgcctaggattatgggtccacatagggaaaacctttaggaaaagaaggatgctggtttccataaacagttcataatca
ccttggaccagcagttctggagaacagaggttctgattcaaatcaggccttgaggtctcattccccaaggagtgggaggcatgta
agcccaggggacaaagcaggactggcctcgaggctggagccatgtgccaatagccccctacgtaccaaccttatttacatggt
ggtgcggggtgccttatcattaggagtctttcagttgtgagggattgtaaatccaatcaaaactagcctaaagagaaggaaatatat
tggcttatatataattggaatgggaaaaaattgaaaaatcaaaatacagttcacatttcagttatggatggcgttgtggcttgaattgt
gccccccccaaaagatcagaagttctaatttctgatgcttgtgattgtgactttattttgaaatagagtctttgcaaatgtaatcaaattg
agatgaggtgctacctgactagggtgggccctacttcagtatgaatgatgtcctgataggagaaaacacacactgacacagaca
acagggagaaagctatttgaagacagacacagggattggagtgatgtgtctacaagccaagcaacgccgaggactgctggca
accactagaagctaagagaaaggcacagaacagattctcccctaaagccttcagagagcttggccctggcaacaccttgatttt
ggacttctggcctcctgaactgtgagagaatacatttctattgtttcagccacccagtttgtggtgctctgcagccctggcaaatgaat
atagctaggcttagaggttcatgaatgtccccaggacttggtgactttccatctgtcaactctgccttcctttacattggttctgtgtccaa
cctctacatagcagccagatcacagccagtaactacagagttggacaagttgcacatcctttatctgaaatgcctgggagcagaa
gtgtttcagatttttggattagggatgctcaacctgtatatccttccagaagcaagtgcaaaggagaaggttgtgtttctcttcaaatat
ctcaacttatgtctgattattctcaagggactttgactgggtcacgtgcctatcagagccagtctccatgatcgtggggtaccaggcct
gagttaagttgcctcctctagaacctaggtgtggagttcttcagaggacatgaactcagagcttgtaatggtacctcttccaggtcca
gcaggccccacgggatgctaatagaagagagatgattggcatgaacaatgaagggtccaacattgccttcaaatctcagttcca
aaggggttttgatacattattatgatggtgctttaaaaaatacagaatgttgtggatattttgaagacatcatatgtggaaaaaacagtt
tctccctagagcagagattgggacttctaggacaactttcccagaggagacgggaagtgtcagtggtaaggaaatgacagagt
gggtggatggtgtggaaagctatcacagacaagaataattttattaccagcattaccaattatacagcacttttcttgttttctcacttga
ttttataataaccccatgagcaagtaagggagctccagatcacgaaatggggctcagaggtgaagtgacatatggaagatgac
ccagctaacacatggagaaactgggattgacttcagacctttgattccaaagctagtgctcttgtaactttctcactctttctaaaattc
acgcattcattcagtaaatactttttcaatacgtcttatattcagggaactatttagtatgcagaatgaaaccttggttataaaaaaaag
gagagagagagagtagtaacatcttcagggctttctgtgtgatcgatatgatgcttagggtctgtatacatcatcataagtatcttcac
gccagctcagtgagatgtgatcacccccagattccagtggagctatccaagcctgagagtggttcagtcagttggccaagagca
gaggtagccgtgggagggctggggtttggttccagctcagtccaatgccaatgcctgtgctcttaattatgttgcctctgctatactcat
aactctgttaacagccataaatccagctctgtctgttagacccagtaaatttcaaagtagaaaatcatttttctaataaaactacgcat
agaaaaaaagatattaatgctcatacattctaccctcattatgacatcaacctctgagccaaactattttgcacattataaagagctg
tttttatgatgaatgggaattatattggcactttaattgagttagaaaccaaggtacatgaatgttagtgcacaagaaatgcgataaa
aaaagctgctcaatgtggttggaatacatcagattaatttaataccaactttaaatccttacaatctatacccttaaatatgttttatcaa
attattagatgaaatttttatactgtttttttttcttttaggtaggtacctattgcacattccccccacccctgcttttatttttttaagac
ggagtcatgctctgtcacccaggctggagcgcaatggcacaatcttggctcactgcaaccttcgcctcctgggttcaagcgattctc
ctgcctcagcctcccaagtagctggaattacagatgtccactaccacgcccagctaatattttgtatttgtggtggagatgtggtttca
ccatgttggcaaggctggtcttgaactcctgacctcatgtgatccacctgcctcagcctcccaaagtgctgggattacaggcatgag
ccaccgtgcctggccctgcattcttaacaaatctgctatatgataaatttagatttcaattttgtgatcaaaactctttttttgctataaaat
gaaactattgccctcttagcttcaaatatggtaatgtaggaggttggcatatatttggataaaattatgtaaacttaaaaaaaaacact
ttccacaataggatgttttaatattggttcagtttcagccataattaatgatttattttatgtcttttgttttagttcaaattagttcatcattaaaa
aaaaaaaactgactccattcagtgcccatacaattagtacttgtgtttgcttgatttagcattctgcaaatgaaagagagtttgttttaat
ttagggcctgtgctttccttaaggtcaaatctccatttgagagaaagaatatggtatttaaataatttagtcaaattggaggccttgaga
caagtcagagtccccaggctttctgaaaatgagatgtcccacgtttgcacttttccagcccaaccaaaaatgatagagttgtcagc
ataaaagttaatgtacaacatgtggatttttaaaacatgattgggatgagtttttgagtaattaatttgctgaaattgtgttgtgctttagcg
cactgtactacaatattagcattgtgaagcgtgcattaaatagttcctgtcaattatggttggctgtgaatgaatctgagggttccttttgt
tataaattactatttcctaaaatggttttgcagagaagcaatggaacacttttagatttggaatgtttaaagagctgttcttgccagtggtt
gattttgagtgagctccaatgtttatgagaactcataaaacaaagcaaagtggggatggcccatttgctgttactccttttcctcccac
tgaaatttccctccagtttttggtggtgcctctgccacagttagctcatctgataaagcagggtgatagctgcctggccacgtatctgat
gataatgatatgagcttttgcatacggtcccttgatcctgctagggccccacccccattctgagcatgcaacattaacataaaaact
accacgccttttgcagctgtggataaaccccaaattccacagctgggggtcacaagagaaagtttagctgaaaatgtatatacct
aaaactggaagttagagggagggttatgaaatatttccaggtgcaatgtatgaatttacagggaattctttttgctgtagttagttatta
ggcaaacagcgctgttcattggtttggcaagagttcctaggttttgcggatagttctctgggtcatttaggaaaaggggtgtttggaag
atgaccctgtgagagttgagatattttgccatgatcccctggtggcagcacatcagaattctgcaggtcgctttgaggttctttgttttgc
cttctcccttgattcttccttctgttcttatggctcaccctgcctttgttttgccatttaaaaataactagcggcccactgacggttttgccag
aggcccttggaaatctaaccgtcaaataaattttattggtgttgctgctgatttttaaaatgaattctctgcaaataggcagaagttact
gccagccagttttgatcaccagcacctttttgcttcaacagttcccagcagctaacacaataatggggccatctttatgtaaatagac
acaatagtttatgtttctaccagctccagaggggttcacagtgttgatcttgactttcagatgggcctttctgagctgagggaggggttg
ctggatgggagaggagcttcccaggagaaaaccatgggtgaataatctcaaaacggttgttgcagctacactcgcatttggaggt
taatttagaaaaagaaaagcaagattggacatcggaatggggactgcagggactgggccgagctaattatttcaaactggccttt
caggccatcctagacacagattggccctggatgggcctcggtctctggtctcttgaaagcccttgcctggtaggaagaagccgctc
tgccaggcagcggaagggagaggcaagcagtgtgagcccatgacgaggcttcagtttatggtttacttaggcttgaaaaggga
aaaatggtgctaaattagatgtgttctggaatcagatggacactgttagtttcctctaaatttccttggccccacctcctttttgtgctttatt
tttgcacacctatgggccccagtcttttagcttcctcccatagattcttgattatttaggaaggaatctttccacacaaaaaggaccatc
aagaaatgggatttatgtccgcagactcggcctgagaagagccgttcatctcagctcagggctgggagggagctgagcaggtttt
cttgcaggagcgatcaatctgccaccagatgtctctgtagcccactctacaggaatgctcacaaacaccagggctggagcctga
gctttccggtgaccttgtggtatatgctctgaattaataaatgaagcagaaatgactgtgtgtgtgtgtgtgtgtgtgtgtgtgtgtatac
gagtgcacacgtgcccatgtgtatgtattttctttcctgagttgcttctcagagtattcccctaactccttggttatctctttccctacactga
gttccttcctaaaagtcagagaagagttgtagggtgctcccagaacgggagattcatcattgataggtgcaagcaaagacagtg
gcagtgggccctgataatctctgtctccttccctaaggtggctcttgggtgcagttatcatgctagggacaggtaaggaatgtcactt
aatcctgggctccctgctggtccccagccaaccagcaaagggaaactcaggtgctgctaggggatgtcattgttgaagggctgc
ccaggaaggctgaaaacaaggatttgctttactgcatgtgtacattcattttagaagctttaaagtatttcaatggaggagcaactta
gcaagttaattaggcaaattaaaaatatgtctaggaaagagagaattaatggtgaatgtggtatgagcctaatctatgcagtggga
gatgctgtggacactccaccagtttgatcacaaagaattcacagaaagcaagccgcgcacggtggctcacgcctgtaatccca
gaactttgggaggccgaggcgggcggatcgcttgagtctgggagttcaagaccagcttgggcaacagagcaagacactgcct
ctaaaaaaacaaacaaataaaaattagccaggtgtggtggcacaggcttctagtcctagctactggagaggctgaggtgggag
taccagaaggttgactcaggaggccaagggtgcagtgagccatgatcaagccagttctctccagcctgggtgatagagcgaga
ccctgtctcaaaaaagaaaaaaagaaagccagccagataaaatgtctggctaaattgggcatctccccaagtccggctggtctg
tcctgagtacagtgaagtcagctgttactgcccttccatgtggagtttgatatgagcagcaaatgatctgacacatacactctctata
aagcatgcttctcagctgtctcactgccatagtacagagaaaaggtgtggcacattcggcgacgtcaggcatgaccacacagag
acacagcccttgaggaacaaggtgactgttcgcaggaggcgttgctcatctgcttatctgattttagttgaattgtctggcaaggatc
ataacagatttaggaatttttccaaataaaaggctgggatacaaaaataggaatcattcagtgggtgagttggtatctgaagaaaa
caagagaacatttaatacagaacagtcctatctatacatgtatacatagacacaaaatataatccagcaagattcacacacagc
atatcactgtcatcaacagtgactctctctccctaatatagggtggaaattgggatacttatgatagaatcatgagatgtagtcctgat
atattccgaagatgtagccttgggaattttcatagatctttcctcccccagaggtcacacacacacaaaagcatcacgtcttgttttac
aaacataagttgaggctggatcttctgaaaacaaaatggaaacattggtgtcgagttggagtgcttgcagtggaccgtgatgcgct
ctgattccttcttcacagTTATTGGAGGGTGGTGAAGAGAGGATGGAGACACCCCAGGAAGACCA
CCTCAGGGGACAGCACTATCATCAGAAGGGGCAAAATGGTTCTTTCGACGCCCCCAAT
GAAAGGCCCTATTCCCTGAAGATCCGAAACACTACCAGCTGCAACTCGGGGACATACA
GGTGCACTCTGCAGGACCCGGATGGGCAGAGAAACCTAAGTGGCAAGGTGATCTTGA
GAGTGACAGgtgaggtgacctgctgcacttgttttcttcttgaacaatgcatgtgtacttcctttaggtcctaaaatcgttcctctctt
ttggagtgtagctctagagctttggatcacatctgtggctgaaagtggaaatccgctgcaagcatgtcaccattttctctttctgtggctt
aaatgatgccttttgtttgacttttgcccaacacttgttaggggctgagggtggaaatgataaaaatgtggtcacagagcccctgattc
cgtacaaccgttgatttctccttctgtcagggatctgaaaggaattggacttcgggtaatattattacacctgcaagagtacagtccct
gtttaagggggcagtgtgtgctttttgcttagtgttgtatgcacacacctcccttaggccctcctggatctccagcccttcatcctggtttct
ttgtttcctggtacttagtacaactggcatgttatgtatggattgatttactgtctgtctccccaagagaacaagaacctctgcgttttcttc
tctgatgtatctgggcacatagtaggccctcaatgaatattcacctgaatgagaggaaccttgcagaggagagtggagagggca
ggcatgtcctgcagggagtggagagaaaatgaagagaatagctgattttctctccttttcctcttccatggcgatattgcctacaactt
aaggggtcagagtctacagtcacttagatctggctcaaatattaactctgccttttgttagtgtgtgaccttgagcaaatcacggattg
acactaagcctcagttctctaatctctaaaatggaagtaacgtctacaacataggcttgttgtgaaggttaaatgagaagttgcgtta
aaatgctgagtgtagtgcccgggatagactgaatgaccaatacatattagggaccatgaggacaatggctctcattacccacgg
ctgtgagaatccatccctcgactgctgcacaaaatgtcgaatccattttcaggggttgacatctctggagatctagccattggctcca
atggcagaaccccctccgctcacttgcactccacctgctcctgcctggggcatcaagcagattctgtttgcaagcacactatagcc
aaagctcaacttgcttccccaaacagcacattgggtgttgcacctgagtggggagaggcacctcccttcatgtctgtccctgggcta
aaggcctcgctgctcttaccctcccttcgtgctgcaccaaaccctttaacagccctgagggagttgttcttccacccaaccatgctgg
cacccttgccgaaagagcttgaatgattctagaaaaatctgttgacgtatttggcaatatcagggcagctcccctgcttcctttcatag
tccctgaaacctcctgaggtgaggacacaccacagtctacccaacagtgatgaagttaagataatttctggattaacaagtggtg
gttcatctggtaggaggacaaaataagccaggaaaggcttgacatccgaagtgcaggcagacaggccgcaggataagcctg
gaccagctgtctggctcagccgccttggtccttggtccttgaccctctctgagcctcggctctttcatctgtaaaatgggactactcag
gcctgttctgaagattcggaaagatgccatgtgagagtcgcatgcagccagacacagagcgacagtgcgcgccggctgctgct
gtcaccttcactgtcactgttactgtcgttcatcctgatggtgggaagaggagacaagcaggactccaggaccaaggaacaaag
cattcttagctttttttcatggtagaaaaatcctgttaaaatggcttcacatgtcgcttacttttttaaagGATGCCCTGCACAG
CGTAAAGAAGAGACTTTTAAGAAATACAGAGCGGAGATTGTCCTGCTGCTGGCTCTGGT
TATTTTCTACTTAACACTCATCATTTTCACTTGTgtaagtatcttcttaaaacatcttctcttattaaaagattacc
cagggcaccaatccaagtatctcttgcagatagtgcgaatcatttaataatggtgagagagattattctttgaaccctggactttttga
ggcccctagactgggagaatcattacaggaagctccctgaaatatttccagcttttgtctagtggctacgtttagagcattgtggaaa
aaaaaaacaaagtaagatataggaaggacgtttgggaaatgacaaggggttctatgcaagagcagaggccctgtaggcgca
gtgctagaagttgcagcgctgagggtcccccatcccagagcagaggccccgctcttcctgtgggtgagggagtgggccccactg
ccccagggatgccaggggatagatcagcctcctttggctgccttcaaactatttctcgtgggggttctccccttctatttttggtatttctg
cccatgccttaagaattaatcccaagaagccagagcagtgaggcacagtgggaggcttccggggtgcaggatggctggccgg
tgctcaggcaccctagacatgcccatgagctgttggttgcaggttctggctcaaagccctcagagattctttctgcatggctgctcac
ctgtgttgatgatggttgtgggagagtagggccacatgtgtgtctgacccctctaggaagtgatctgccccctttgtctccatccacca
ggcagggctggctacctaggggccaggacagacttcacccaggagctaccccaggactggttcttgccactcactgtgtccctct
attcacttacttgcctctctggctgtgcactcatctctctggtttctattttagataccagtcaatcagagactccagtgagcacctactat
gttcaaggcattatgctaggcactgtacagggcataaaaaggtgtaagacattgttcctgccctcaaggagcttacagttaggatgt
tagggttatttgcgtataagaagataattagagttaccaggcagtatgttttaaacatgaatgactttagcttcttgttggaaaatgcct
gcttctgtgggcattgactttccatacagagacctaacagtagggggtcgaaatggccacaatcagtgaatctcctggtccaagttt
agagacgccagtgaaatggttggtacaaatcccttgtggagcgagtgaggcagtgagtatgagagcttccagaatgggttgtcta
gccagctcttagtgaatagagtttaaaaggaggtgacaactgctgaatttttccaattattcacttcacatttctttcatttctttttagAA
GTTTGCACGGCTACAGAGTATCTTCCCAGATTTTTCTAAAGCTGGCATGGAACGAGCTT
TTCTCCCAGTTACCTCCCCAAATAAGCATTTAGGGCTAGTGACTCCTCACAAGACAGAA
CTGGTATGAGCAGGATTTCTGCAGGTTCTTCTTCCTGAAGCTGAGGCTCAGGGGTGTG
CCTGTCTGTTACACTGGAGGAGAGAAGAATGAGCCTACGCTGAAGATGGCATCCTGTG
AAGTCCTTCACCTCACTGAAAACATCTGGAAGGGGATCCCACCCCATTTTCTGTGGGCA
GGCCTCGAAAACCATCACATGACCACATAGCATGAGGCCACTGCTGCTTCTCCATGGC
CACCTTTTCAGCGATGTATGCAGCTATCTGGTCAACCTCCTGGACATTTTTTCAGTCATA
TAAAAGCTATGGTGAGATGCAGCTGGAAAAGGGTCTTGGGAAATATGAATGCCCCCAG
CTGGCCCGTGACAGACTCCTGAGGACAGCTGTCCTCTTCTGCATCTTGGGGACATCTC
TTTGAATTTTCTGTGTTTTGCTGTACCAGCCCAGATGTTTTACGTCTGGGAGAAATTGAC
AGATCAAGCTGTGAGACAGTGGGAAATATTTAGCAAATAATTTCCTGGTGTGAAGGTCC
TGCTATTACTAAGGAGTAATCTGTGTACAAAGAAATAACAAGTCGATGAACTATTCCCCA
GCAGGGTCTTTTCATCTGGGAAAGACATCCATAAAGAAGCAATAAAGAAGAGTGCCACA
TTTATTTTTATATCTATATGTACTTGTCAAAGAAGGTTTGTGTTTTTCTGCTTTTGAAATCT
GTATCTGTAGTGAGATAGCATTGTGAACTGACAGGCAGCCTGGACATAGAGAGGGAGA
AGAAGTCAGAGAGGGTGACAAGATAGAGAGCTATTTAATGGCCGGCTGGAAATGCTGG
GCTGACGGTGCAGTCTGGGTGCTCGCCCACTTGTCCCACTATCTGGGTGCATGATCTT
GAGCAAGTTCCTTCTGGTGTCTGCTTTCTCCATTGTAAACCACAAGGCTGTTGCATGGG
CTAATGAAGATCATATACGTGAAAATTATTTGAAAACATATAAAGCACTATACAGATTCGA
AACTCCATTGAGTCATTATCCTTGCTATGATGATGGTGTTTTGGGGATGAGAGGGTGCT
ATCCATTTCTCATGTTTTCCATTGTTTGAAACAAAGAAGGTTACCAAGAAGCCTTTCCTG
TAGCCTTCTGTAGGAATTCTTTTGGGGAAGTGAGGAAGCCAGGTCCACGGTCTGTTCTT
GAAGCAGTAGCCTAACACACTCCAAGATATGGACACACGGGAGCCGCTGGCAGAAGG
GACTTCACGAAGTGTTGCATGGATGTTTTAGCCATTGTTGGCTTTCCCTTATCAAACTTG
GGCCCTTCCCTTCTTGGTTTCCAAAGGCATTTTATTGCTTGAGTTATATGTTCACTGTCC
CCCTAATATTAGGGAGTAAAACGGATACCAAGTTGATTTAGTGTTTTTACCTCTGTCTTG
GCTTTCATGTTATTAAACGTATGCATGTGAAGAAAGGGTGTTTTTCTGTTTTATATTCAAC
TCATAAGACTTTGGGATAGGAAAAATGAGTAATGGTTACTAGGCTTAATACCTGGGTGA
TTACATAATCTGTACAATGAACCCCCATGATGTAAGTTTACCTATGTAACAAACCTGCAC
TTATACCCATGAACTTAAAATGAAAGTTAAAAATAAAAAACATATACAAATaaaaaaatcccga
ctttgggatgagtgctaggatgttgtaaaccagtttgagaatcagaatccaaaatgagagctgaaagattggctgagtctttctcgg
agggagggcatgctggcagacagagctttgtaaacagcatcctccttcccagagatgcttctgcttccatcctggggccacgttgc
tacccagtacatgagcagctcatactaacatgcacggtcatgggtgggcgggatggagggagggtttctgcttcagaaagatgtg
taacatcaggggctttgtgcctggattcatgggtttcactcaagattctcaaataggtcccttccccccaaaatgttaagaacgatgtg
gtctaagtagttgtaatagttataaaagcatcaggccaggcacggtgactcatgcctgtaatcccagcactttgggaggccgagg
caggcggataacgaggtcaggagatcgagaccatcctggctgacacggtgaaaccccgtgtctactaaaaatacaaaaaatt
agccgggcgtggtggcgggtgcctgtagtcccagctactcaggaggctgagacaggagaatggcatgaaccctggaggcag
agcttgcagtgagccgtgattgtgccactgcactgcagcctgggcgacagagcaagactccgtctcaaaaaaaaaaaaagcat
cataagtggaagtctctttacaaagatgaatacacataaaatgtctctaaaagctgtggaatcactttcaatggaatcaagtctgttc
tcaaatgctttaccaaaagtgccagggcatggtaattgagagttcacagagctcctagtcacctgagtgtgtagcccagcttcaag
atttggaagttatatttccttgggcagaggacttacccctctaagccttagctggccaatctttaaaataagaatagtatctgcctaata
ggtttattgtgaggattaaataagataatatatagaagcagtaagcctagtgtgtagcaaaaggtaagcctttgactgatattagaa
caagaaaggagaaaaaggtagcagagaaagtatcagtaaccataaatctttgacaaagtggttttgttaaaaggaatgaattgg
cttggtgaaggagtcatgctgctttcagaggattaatactcagtgtactaaaattcttcgtggccattagaattacagtacaggacac
accaggaagaagggttgccctttgtcagtttggactgaattaagctggaaacatgatggaaatttgagagcaggcggactcaatg
tttcagacctagtctttggtataagaaaaagtttgtgtgtggcggggcacggtggctcacatctgtaatcccagcactttgggaggcc
aaggcgggcggataatgaggtcaggagtttgagagtagcctggccagtatagtgaaacctgtctctactaaaaacacaaaaatt
ggccaggcgtggcggcgtgtgcctgtggtcccagctacttgggaggctgaggcaggagaatcacttgaacccgggaggcgga
ggttgcagtgagccgagatcgcaccactgcactccagcctgggcaacagagtgagactccatcttaaaaaaaaaaaaaatgt
gtgtgtgtgaggcagagagagagagagagagagagagaagggggtgtagaagagaatggagggcagaatttgtcaagga
gagtggactggtctcaactgcctcgattgaggcctacgaagatgtttcagaggaaggcagatgatcatggaccatatttattcttcat
ctccattgccagggaaagctttgtattcaaggctgtcccttgtctatgaaattagttctagagttataataattttgccttgggatgtccca
gggcacaaatacagatgtgactatcagctccacattcttccaaaagaaagcctgtggttttttcgtatttataataatacttaggaggtt
tcctcgtagaaaatac
HRH1 genomic sequence

SEQ ID NO: 4

aaagcatctcataagggggtagacctatgttttttcagggagcagttcggactctcaacagggcaataggcctttcgactctccctg
atgagggtggatgcacggcatgtggtactcccattttctttaggttgtttgttggtttttctgcgcactctgaaacgatctgcaacttgtcta
gcaagggtataaattcctacgcatccataaactctgaggactgcatcacacatagcttgggggccccagtgagttccttgatgtag
ctgtgacaatacctctcgcatgaagggcttagacagcatttccttcttgtctggttgtacccatcttccctcgtgactttctttggctcctatt
tcttttaacttttctttttctctgggggagaaaacagggactgtagctggagggggaagataaggggttaggtggaaaatgggtgct
gcttgagaagaggcaacatgtttagccacttggtcagctagattattccctcagctctcaaaggaaagattcttctagtgacctgga
acatgaacaactgctgtctcttctggcggctgtaagttctctagtacttggattatcaagtctctatggactaagttttggcctttactgtta
ataaggcctcgctctgcccaaattttcccaaaggtgtggactactccaaaggcatacgtggagtcagtataaatagttccttcctggt
tttgcagaaattttaaggcttgatttagtgtaaacaactcacatgtttgctcagaccagtcattaggtagggtgtctccgtctactgctga
gtacctgttatgccttttcccttttattacttgagaagaaccatctacaaaaaggtgtcttccggtttgaaagggagtttatctaaacatct
atgcccaagttcttctggtctggggcatgggtttttttctgcgtttgaatttcctgttaagaaggcagcagggttaagtgaatcatctgta
gttcggattaaatcatctttttctaacaagatagccttgtattttaaaattcttgagtcagtaagcaacctttctgccttctgatttaggatag
ttctgttctggtgaggtgtgctcacaatggggtttcctccaaaagttatttttctactttcttctgttagcaaagtagttgccgctacagattg
aatgcatttggaccatccatgggttactgggttaagaatttttgacaggaagcctacgggttgggagtggcctccgtgcttttgggta
agtactcccaaggctacgcccttgtttacattgacgaaaagatggaatggctgcttagggagggtaaagctaggacaggggcag
ttactaatagatgttttaacctttctaccttttggatttctggtaattgccaaatgatggggtctggctcgtcttgtgtgagctttttgtatgag
agttctgtttctagggcataagagtctatccatagacggcagtatctgactaatcctccttcaatccattcaagcccaattttccatttgc
ctttgtaattaaatgccctaaatattttacttcaggttctacaaattggagtttgtttttcgaggccgttaacccttcatcccacagaaaatt
taagacatgggttgagaaagctgctacttcttttctatcatctcctgaaattagaagatcatccatgtactggaggggacatatgcac
gagggcagggaaaatttctctaggacttgttctaatatttgactaagtaaatatggagactccgtaaacccccggggtaagactgtc
catcagtattgctgttttcaaccggagtgagggtctttccactcaaaggcaagtaggtcctggctgccctctgctaatggacaagcc
cagaaggcatcatttaaatctattactgtactatctgattaatagctctaaggtcttgcactaaccagtatgacctgtctggcttctttaa
aggcagtattggagtgttaCAGGGAGACATACAGGATTTAAGAAGCCCATCATGGAGAAGACCTT
CAATTACAGgttttaaatttaccctggcttctaaaggaatagggtattgctttctctttactacttccccaggggttttaaatttaaca
tgaatcagagaaatctgtaactttccttgatcccatcttttgaccatacctcgggataaatgtgttcttcgtctgcggtggtgagcaaga
ttagggaggggaggggaggaattttccgtgattgatttggaggcctaagcctaattttagtattaaatcacttcctaatagatttgtccc
tgcttctggaattaacagaaatttgctgctagctgatcagttttcatatttcacttttgtctcctctaagatttttgctctaaatccttctcctttta
ctccagAGATAAAAAGTTTTTCTTGTGAACAAGTTACACTAGATGGAAGATAACAGACTGAG
GAGTGAGCTGCTTCTGACTCGATTAAAAAGgtaataagcttaggtttaggtcccacttctaaatttaccaaggg
ctgttggtgggactcaagagacaaagatggagcccctgacctccctagtcttcttcaaaagctgtaagtgggatgactttttcttctttt
tcccatttgggacattgtctttcaaaatgacctatttttccacatttgaaacatttgttctgtgatttcttagtaatatctcgccagctattggt
gacaaagtggagctttaacattccttgtccgagggggtcttctgattctaggccagcgtatttttctcatttgctctttaagcctttctaaa
aattctgtcggtccctcatcttttccctgttttatattaaaggccttggtaagattttgggtgcggggcactaattctcaaattccttttattac
catctccctaaggtctctcatatttccttgatgggctatattgttgttatctcattgaggatcctgggctgggaattatgttcagccgctgg
aacgttctgaccgggaggatgttcacgttcccgaatggtcatagcagccctttgtatcatgctcctttcttctcctgagaataagatgtc
taagatagacattaactcgtctaaagtatatatctggggtcctaaaactgatcgatctgatctgccactccataagggtcatctaaga
gtggtttaagctccttttttaggtttaggggaagggtctgtggtggcagctgcttgggggagaggaaggttcttcagtccaaacaaaa
cagcagttttttatcatttgctgctttttcttgtgtttggtccttccattatccttccaatattctaacgttaggcctaggggactatcagaggg
tatattatcatgatcatgatatttcctatcttttgtcttacttgctgtatttcccatcctggagaaagagtttttccctgagtccatggggctca
atctctcttactagagatttcttgcaccctagtgagtctgtggggctcaacctctcctactagagatttttcacactcttcagcttttgcttta
tccttctccatatgcttctcttgcggaaattttcaagtccctcttagcataggcaggttggtataaaccccacaacaggcaagctgcctt
taagccatatgaggtgactacagaaccagatccggactctgcacttgctctgcactcaattgtgtgtcttactcacacactttcaacct
ccaggatgtcctgaccaccaaggaaatacttcactgcccccaaggtttttcttaccttggtctatgcacagagttacctggtcgccac
agtatctgtctgccttttcttccctcattgctagagtccaggtttattcatcacaccaggtgggtctcgatcccttacccttgaggccacc
gcaacaaagcagcgggctgcgtctcctcacgagaaatgatctgagaccctccccggaggagaatgggaatcccagatgaac
ccccaagtttgttagaaacaagtgcctggtgccacaaagaaaaacagcacataggcagaaaattcctcagcaaggcaaattta
cttctgcagaagggtgcagcttgtgctagtcacaatcgcaagagcacaccaagcagggtagggcaggggtttttaatccctaatg
cagttcctagcacttctgtgtcctttccgcattggctggggttggacttcacaatctaagctaattcgattggctaagatttaaaattgaa
tagggtctattaggtgggaaggaagaggaactatccgttactaggtgggaaggcatatctggacttgtctgggcctggcgaaggc
aggaaggctgtttacagaacaggtagctaggagacaaggatgtacaaggaagttggtcttaagaaacaaagaacagagaac
taaacctttttgaagaggaatttatcatctctgacaggaggctgcagtgagctgagatcacgccattgcactccagcttgggcaatg
agagtgaaactccgtctcaaattaaaattaaaattaaaaaataaaaaataacgtaaaataaaaaatggtttctctcccctctatgtg
ccagacaatgaggaaaagagaaaaaggagacacctctggaggccagggagctgagagccaccttgagaatgccaagctg
gggaagtgtttaggggaactacttcctgcttccttccgagcaaaacagtaaaaaataaaaatccctgagacaatacttccttagcc
ttatgaaccccgaaaatctgagataggtctcagttaatttggaaagtttattttgccaaggttgaggacgcacacccatgacacagc
aacaggaggtcctgacgatgtgcccaaagtggtcagagcacagtttggttttatacattctagggagacatgagacatcaatcaat
atatgcaagatgaacattccttaggtctgggaaaggcaggacaactggaagccgggaggaggcttccaggtcttaggaagata
agagacagatggttgcattcttttgagtttctgattagcctctccaaaagaggcaatcagatatgcatttatctcagtgagcagaggtc
tgacttcgaacagaatgggaggcgggtttgccctaagcagttcccaacttgacttttccctttaccttaagtgattttggggccccaag
ttattttcctttcacagcctactttcttccttccagaagtgactgtggacaattccacagggtttggacttgatcagggcagaaggtgaa
gctgcaaggtattagatgtgggaatggagaaaaatacaggctggagctgtgggtttgagtgttgtcctcataggaggtgatggctg
aggggtaggtaagtgagaggatgagatccccgaggccgacagcacagagtgacaggagcatagggcaggactttgggtca
cccaaggagacagtgatgcttttgaagaagtcagaggaggccccatcagcaatcagaggattgctctgattggcacctcagag
ctggaggacatcaaaaaataccgctgtaagaaagagacctggaaaagtctttagagattgtctatcccaccctacccatttgaca
catgagaagatggaggccaagagatcactgagaaaataaatggtagagcttgggcaaaatcagtgctgcccaaaatggtgtttt
tccaacaaagacatttaaaaggttccttccacaaggatcaaacaccttggggttttgatttttatcttaaaaagttatataaatttagcct
tctacaggccaggcacggtggctcacacctataatcccagcactttgggaggctgaggtgggtggatcatgaggtcaggagatc
aaaaggatcctggctgatatggtgaaaccccatctctactaaaaatacaaaaattagctgggcgtggtggtgggcgcatgtaatc
ccagctactcaggaggctgaggcaggagaattacttgaacctgggaggcagaggttgcagtgaaccgagatcgcgccattgc
actccagtctggcgacagagcgagactccgtctctaaataaataaataaataaatttagccttctactcaagaacttatctggctttg
tcttaatgtaaaaataatttctttttgctaaattattgagagaaatttactatttattagtgtttatcagttttctttaaactcaccactttttgatg
aatatgaaaatctaaaaacttggccgggcgcagtggctcacacctgtaatctcagcactttgggaggccaaggtgggcggatca
tctgaggtcaggagttcaagatcagcctgaccaacatggtgaaaccccttctctactaaaaatacaaaaattagctgggcgtggt
ggtgggtgcctgtaattgtagctactcgggaggctaaggcatgagaatcacttgaacccagaaagcagaggttgcagtgagctg
agatggtgccactgcactccagcctgggcgacagagtgagactctgtcctaaaaaaaaaaaaaaaaaaaatggctgggcgtg
gtgcctcatgcctgtaatcccagcactttgggagtccagcgtgggtggatcacctgaggtcaggagttcaagtccagcctgacca
acatggtgaaaccccgtctctactaaaaaaatacaaaaaaaatagccgggtgtggtggcacactcctgtaatcccagctactca
ggaggctgaggcaggagaatcacttgaatttgggagctggagattgtagtgagccaagatggtgccattgcactccagtctgggt
gacagagtgagactccatctcaaaaaaaaaaaaaaaatcttaaaaactccttccagaagatttaatacttactttcacccaacca
cccgacttgagtatcaccaataacagaggatacagtccgttttcagtagagccttagtagcaaagggttttcatttttatttttcagata
caggatcttgccctgtcacccaagctggagtgcagtgatgtgatcatagctgactgcagcctcctgagtagctaggactataggtgt
attataggacaatttttaaaaaatttcattgtaaagacaggattccactgtgttgcccaggctgcaagtcttggcctcaagtgatcattc
cacctttaactcttgccctcaagcaatcctcccacctcagactcccaaaatgctgggattatgggtgtgagccaccatttccagccta
ctagcaagggtcttgttacatattacttggcatgatttatgtaatttaaaaaaattgtttgtttttcaaatagaaaagtaaaataacgaat
atgcttttccaataacataatccccttctcacttgagaattttcctctaaaaagatatgctagatttatttcatgctttatgtgcctctggtgt
gtccccttataacctcctccatatcatttagggatggtctcagctgcaagtaagaactgccacaacagtgatgtaagccaaaaaaa
aaaaaaaaaaaaaaagcaaagccaagcaaaacaaagcccatttaattatttcccataataataagtctgggagaagaagatt
ccagagttggctcagcagcttagtgacagcaaggccctaggctggcattttcttggccttcccgatggtcccaagatgactctcatg
gcctcaaacatcacttcctcacatcctgtcagggagaaagaggcaagtgagcaacaacaatttttgttgttttgatcatttgtcagag
aggaagaacgttcctaaaaactccgcctctgctgtttgacatcctcatcctattccttggccatggtggtatctcatggtcactcctctat
ctgccactgtaaagaggaactggattgctatattctgcttagacacatgaggatgcagcccaccttcccagaacatgtgcggaatt
agatttctacaaacacatttgtcttgcttctgcccaactctctcactagaatgcacattccataggggcaaacatttttgtctattttgttca
cagctatattctcaacacctagaagagtgacagaaattcaataaatagttgttaagtgagcaaatgaatgcatgaataaggaaaa
gggtacatggctattgagtaggtaaccagcagtgttgatcacccccaacagcatacaactccagtctgatgaacatcatgctacta
agtggccactcatcacccaagtctctgaccttactttttctctcttttctcccagGGAGTGAGCCATAACTGGTGGCTG
CTCTTGCGCCAATGAGCCTCCCCAATTCCTCCTGCCTCTTAGAAGACAAGATGTGTGAG
GGCAACAAGACCACTATGGCCAGCCCCCAGCTGATGCCCCTGGTGGTGGTCCTGAGC
ACTATCTGCTTGGTCACAGTAGGGCTCAACCTGCTGGTGCTGTATGCCGTACGGAGTG
AGCGGAAGCTCCACACTGTGGGGAACCTGTACATCGTCAGCCTCTCGGTGGCGGACTT
GATCGTGGGTGCCGTCGTCATGCCTATGAACATCCTCTACCTGCTCATGTCCAAGTGGT
CACTGGGCCGTCCTCTCTGCCTCTTTTGGCTTTCCATGGACTATGTGGCCAGCACAGC
GTCCATTTTCAGTGTCTTCATCCTGTGCATTGATCGCTACCGCTCTGTCCAGCAGCCCC
TCAGGTACCTTAAGTATCGTACCAAGACCCGAGCCTCGGCCACCATTCTGGGGGCCTG
GTTTCTCTCTTTTCTGTGGGTTATTCCCATTCTAGGCTGGAATCACTTCATGCAGCAGAC
CTCGGTGCGCCGAGAGGACAAGTGTGAGACAGACTTCTATGATGTCACCTGGTTCAAG
GTCATGACTGCCATCATCAACTTCTACCTGCCCACCTTGCTCATGCTCTGGTTCTATGC
CAAGATCTACAAGGCCGTACGACAACACTGCCAGCACCGGGAGCTCATCAATAGGTCC
CTCCCTTCCTTCTCAGAAATTAAGCTGAGGCCAGAGAACCCCAAGGGGGATGCCAAGA
AACCAGGGAAGGAGTCTCCCTGGGAGGTTCTGAAAAGGAAGCCAAAAGATGCTGGTGG
TGGATCTGTCTTGAAGTCACCATCCCAAACCCCCAAGGAGATGAAATCCCCAGTTGTCT
TCAGCCAAGAGGATGATAGAGAAGTAGACAAACTCTACTGCTTTCCACTTGATATTGTG
CACATGCAGGCTGCGGCAGAGGGGAGTAGCAGGGACTATGTAGCCGTCAACCGGAGC
CATGGCCAGCTCAAGACAGATGAGCAGGGCCTGAACACACATGGGGCCAGCGAGATA
TCAGAGGATCAGATGTTAGGTGATAGCCAATCCTTCTCTCGAACGGACTCAGATACCAC
CACAGAGACAGCACCAGGCAAAGGCAAATTGAGGAGTGGGTCTAACACAGGCCTGGAT
TACATCAAGTTTACTTGGAAGAGGCTCCGCTCGCATTCAAGACAGTATGTATCTGGGTT
GCACATGAACCGCGAAAGGAAGGCCGCCAAACAGTTGGGTTTTATCATGGCAGCCTTC
ATCCTCTGCTGGATCCCTTATTTCATCTTCTTCATGGTCATTGCCTTCTGCAAGAACTGT
TGCAATGAACATTTGCACATGTTCACCATCTGGCTGGGCTACATCAACTCCACACTGAA
CCCCCTCATCTACCCCTTGTGCAATGAGAACTTCAAGAAGACATTCAAGAGAATTCTGC
ATATTCGCTCCTAAGGGAGGCTCTGAGGGGATGCAACAAAATGATCCTTATGATGTCCA
ACAAGGAAATAGAGGACGAAGGCCTGTGTGTTGCCAGGCAGGCACCTGGGCTTTCTGG
AATCCAAACCACAGTCTTAGGGGCTTGGTAGTTTGGAAAGTTCTTAGGCACCATAGAAG
AACAGCAGATGGCGGTGATCAGCAGAGAGATTGAACTTTGAGGAGGAAGCAGAATCTT
TGCAAGAAAGTCAGACCTGTTTCTTGTAACTGGGTTCAAAAAGAAAAAAATAATAAAAAT
AAAAGAGAGAGAGAATCAGACCTGGGTGGAACTCTCCTGCTCCTCAGGAACTATGGGA
GCCTCAGACTCATTGTAATTCAAGCTTTCCGAGTCAAGTGATTGACAACTGAAGAGACA
CGTGGCTAGGGTTCCACTGGAGAATTGAAAAGGACTCTTGAGCCCTCCTGGAATGGAG
CTGTATAACTGTGCAGAGACTTTATCCATGCCAATAGTTGCTGTCCCCTTCCAGGGGTC
ACCTTGAGAGGCATGACAGCTGTTCCACAGGGGCTATCCCTTCTCAGAAAACTTCTCTT
CTGAGCCTCTTTAACAGCTTTCTCCAGAACCAGTGTCTGAACCACCCTGGAAATTCTGC
CTTATTATTTCTTACTCAAACATGTTTAGAGTGGATAGAAAATTATGCAGCTTGCACACC
CATCGTCTTTAACCCCAAATTTCCTTTGGCTATTAAAAAAGTGGTGGCAAAAGACATCCT
CAAAAGAAAGAGAAATGAAATATTTTTGAATGGTTGCACGTTAAAAATTAAAAGAAGGAA
TGGGGGCAGAATGCCATATTTTTGAGGGCTGTACTAGGTTTATCTCATTTAAGCCCCAC
AACACCCCACAGGAGGGTAATTTTCTAACTCTAGTTTGCAGAGGAGCAAATTGAGGTTC
AGCAAGGTGAGAGAGGTACCCAAGGTCACATAGCTAGTTATGTGAGAAAGTTAGAGTA
CAGATCCTCTGGGGTTTCAGCTTATTGTAGCATATTTTCTCCGAAAGGCAAAAATGTGC
CCTTTTGGCCGGGCATGGTAGCTCAAGCCTATAATCCCAGCATGTTGAGAGGCTGAGG
TGGGCAGATCATTTGAGGCCAGGAGTTCAAGACCAGTCTGGCCAATATGGAGAAACCT
TGTCTCTACTAAAAACACAAAAATTATCTGGGCATGGTGGGGCATGCCTGTAGTCCCAC
TTACTTGGGAGGCCGAGGCACGAGAATTGCTTGAACCCGGGAGGTGGAGGTTGCCGT
GAGCCAAGATCACGCCACTGCACTCCAGCCTGGGCAACAGAGCAAGACTCTGTCTCAA
AAAAAAAAATACAATATTTTAACAATGTGCCCTCTTAAGTGTGCACAGATACACATACAC
GGTATTCCCAAGAGTGGTGGCAGCTCAAAATGATATGTTTGAGTAGACGAACAGCTGAC
ATGGAGTTCCCGTGCACCTACGGAAGGGGACGCTTTGAAGGAACCAAGTGCATTTTTAT
CTGTGAGTTCTGTTGTGTTTGTCAAAAAGTCATTGTAATCTTTCATAGCCATACCTGGTA
AGCAAAAACTAGTAAAGACATAGGAACATGTAGTTTTACTTGGTGTTTATGTTGCAATCT
GGTTGTGATTTATATTTTAAAGCTTGGTGCTAAACCACAATATGTATAGCATATGGAGTG
CCTGTACAAGCTGATGTTTTGTATTTTGTGTTCCTCTTTGCATGATCTGTCAAAGTGAGA
TATTTTTACCTGCCTAAAATATGATGTTTAAAAGCATACTCTATGTGATTTATTTATTTCTA
CCTTTCTGAGTCTCTTGGACTAAGAAGATGTTTTGAAATGTACCATCAAATGTTAACAGA
GTTTGATATGGGCTTTCTCTTTGGTTTCTCATCACATTTGTAAATGTCTTTTCAAAAGGAT
TTACTTTTTGTAAAAAGCTTCATTCTCACTCTGCTTTGCATCCCCCAAACTTCTTGTTCAA
AACGGGGGGAGTTTAGGAGACTTTAATCCCGGTTTCAGAAGCTGCAGCTGGTCTGTTT
CCAGGTCAGAAACCATTGTTCAGAAGACCTCCCTGTGAGAGAGTTGCTCCTCAGGGTC
CCTCAGGACCAAAGAACACTCGAAAAGAGCACTTCACACAGACAAGTGGCTAAGTGTC
CATTATTTACCTTGAACAATCAAGGCAACTAGTGGAGAGAACTGATTGTGAGCTCtgcctct
gggtcagagagacctggatttgagtctgacaagaacaagaaatggtcaataaatataaattaccagcgtctaaggaacaaggt
ctatgcattattgtatacagtgtctctagtgcttgtatagtgtctggtatacagagggcactcctatgcatttttaaaacatgctgagcac
ataccatgtgccaggctttgtgttttatctaatgttatctaatggtattggtgccattatgtaatgttgcctttacaacaacctcatgaggga
gatttccatctttacaaataggcaaactgaggcccagagagattgaggaactgctccgaggtctgattctggaatgtgcttcctttcc
actttatcaatctgctcttcgtactcctgtctgaacgatggaaattaatttttgaatgtataaaagacaacagactatgatacagaaat
gtcagccccagcccactaagaaagccccagcccatcagtggctaatggctttaataaattggtcatttggctacttggcttgtggac
aatctctgacctcttttgaagatgggcactgcatggacttccaggaggtggatttaatagtcttaactcagcatgaaaaagatgctgg
gatgctcctggctatttatgcaccctaagtgccatagagacatgctgttggcaaggcatggtggctcatgcctgtaatcccagcaca
ctgggaggctgacgcgggcagatggctggagtccaggagttcgagacaagcctgggcaccatggtgaaatcctgtctctacta
aaaataaaaaattagccaggggctgtgacgcacacctgcagtcccaactacttggggggctgaggcaggaggatcacttgag
cccagaaagttgaggctgcagtgaaccaagattgggccactgcactccagcctgggtgacagagagagactctgtcttaaaat
gaaatgaaatgaaatgaaatataaaataaaataaaatatagaaacatgctgttaaagatcttatttgccaatatttatcattccaca
atttgtcaggctttcaaagcctagcttgacgtgacatataattctcattgtggggagcatgtactcttctcaactcagatgcaagacaa
atgatgaaggtggattgacctgaatcactgtagccttgaataagtgtcacagggcctcatgaccctgctgtgtctgagaacattctct
gcctctttaagtctcctgggtctgcatctttcttaatgctccatggtcttggagccccaatggtctgcctatcccattccaggcagcaga
ggcaggtcttcttccttagcctcaccctatcttcctgctaacaaggaagcctcatttgtcgtctgagcaatcattagctctggtccccat
atctattttggattcccagaccttatctgttaattaacaaatatttttccagcacttcttatgtctggcctgagccagaaagacatgatttc
aaccctgtggagctacttcaggtttgcaagtgacagaaatcaactttgtgaatagttactagagtatcttaagtctgttttgtgctcctat
aacagaatatcacagactgggtaatttataatgaatagaaatttattggctcacagttctggaggctggtaagtctaatatcaatgtg
ctggcatctggtgaggaccttcctgatgcatcatgacatgatggaagagcaaagagagggcaagagagagcaaaagggagc
aatcccactcctgtaataaagaactcactcccatgataacagcattagtgcattcatgagagtggaaacccatgacctaaacactt
cttaaagatcccacttcccaatatgatcacaatggcaattaaattttaacatgagttttggagaggacaaatgttcaagccacatag
catggcatgctttgtggaatctagtgatactttggatgactttgccttgaggagggcttgagacaagtca
IL-2 genomic sequence

SEQ ID NO: 5

gatgtgtcagacgtgagaaagcgaaagtatgtcacagcgaatgtagcttttccacacgtatttcaagaaagaaatgaaaaagcc
aacttctataatggtgcctactgtgcattaacagagataaactaggggtctaagaactcagttttctacagggtcccagaagtatag
ccatatattgccccattctctaatggaaatagccagagaaatagaaatatcaagactggagaacatcaaatacctcattggaaaa
gcccccacataggaaaatgtgtgggcttgaattcttccattctggaagggtaaaggcctgagtgatgatgctgggattagacactg
aaactctttagagaagcaaaacaagtataataaagctgtactttattatattaaataaataacacacagactaccaaatagcctgc
cccttataacagcgttaatgtgattttgatctgaaatgtatagagacattttgcattttttcgtataaaaagttcatgagatttggccctaat
ctgaccttttcttcatttttttttctacttgagggactataatctttatttttaaatttgttttatattctccgaacattacctaacgcatagaaaac
tcttattgaaccatttttctctgttctttgtaaaatattacatttgactgttccttagactgctttaatcattcctgcctatgcaccctcctcaaa
atccagtttaaattaattgttccttattcaagattccttatatccacctcccttggggcagcaatcacctatcacccaggactacacttgt
gtatgtacatatcttccctattacaaatcaggttctttgaaaaaatacaaatggtaagagagtggatttttggagtcagaacattctcttt
tcaaatccttcttctgccccttactggcaataagggctgagtgacctagagcaaattacttaacttctctgagcctcagttttctaatctg
caaaataggagccatcacttcacaagtctgtaagacttatattagactaagtgcctgcctgtacactgttctcttttctctctttctatata
cctgaaggcattataggtgctagatgtctgtttaaagaccagacaatattgtcttaaaaaaacaaacaaaaacacagacaatacc
atctttaaaaaaaaaaaaaaagtccaggtaagaaataaataaggccatagaatggaagctttacaaggactctctctgagaca
ggatctcctcaagtgtccccaggttaaattagaagtatatatccgtacaattgttcagccagtttgtgcactgtactgaggatgaatga
acacctatcctaaatatcctagtcttctgactaaaaacaagatcatatttcataacgattattgttacattcatagtgtcccaggtgattt
agaggataaataaaaatccattaaagaggtaaagacataaaaacgagaaacatggactggtttacacataacacatacaaag
tctattataaaactagcatcagtatccttgaatgcaaacctttttctgagtatttaacaatcgcaccctttaaaaaatgtacaatagaca
ttaagagacttaaacagatatataatcattttaaattaaaatagcgttaaacagtacctcaagctcaataagcattttaagtattctaat
cttagtatttctctagctgacatgtaagaagcaatctatcttattgtatgcaattagctcattgtgtggataaaaaggtaaaaccattctg
aaacaggaaaccaatacacttcctgttttatcaacaaatctaaacatttattcttttcatctgtttactcttgctcttgtccaccacaatatg
ctattcacatgttcagtgtagttttatgacaaagaaaattttctgagttacttttgtatccccacccccttaaagaaaggaggaaaaact
gtttcatacagaaggcgttaattgcatgaattagagCTATCACCTAAGTGTGGGCTAATGTAACAAAGAGG
GATTTCACCTACATCCATTCAGTCAGTCTTTGGGGGTTTAAAGAAATTCCAAAGAGTCAT
CAGAAGAGGAAAAATGAAGGTAATGTTTTTTCAGACAGGTAAAGTCTTTGAAAATATGTG
TAATATGTAAAACATTTTGACACCCCCATAATATTTTTCCAGAATTAACAGTATAAATTGC
ATCTCTTGTTCAAGAGTTCCCTATCACTCTCTTTAATCACTACTCACAGTAACCTCAACTC
CTGCCACAATGTACAGGATGCAACTCCTGTCTTGCATTGCACTAAGTCTTGCACTTGTC
ACAAACAGTGCACCTACTTCAAGTTCTACAAAGAAAACACAGCTACAACTGGAGCATTTA
CTGCTGGATTTACAGATGATTTTGAATGGAATTAATgtaagtatatttcctttcttactaaaattattacatttag
taatctagctggagatcatttcttaataacaatgcattatactttcttagAATTACAAGAATCCCAAACTCACCAGGA
TGCTCACATTTAAGTTTTACATGCCCAAGAAGgtaagtacaatattttatgttcaatttctgttttaataaaattca
aagtaatatgaaaatttgcacagatgggactaatagcagctcatctgaggtaaagagtaactttaatttgtttttttgaaaacccaagt
ttgataatgaagcctctattaaaacagttttacctatatttttaatatatatttgtgtgttggtgggggtgggaagaaaacataaaaataa
tattctcactttatcgataagacaattctaaacaaaaatgttcatttatggtttcatttaaaaatgtaaaactctaaaatatttgattatgtc
attttagtatgtaaaataccaaaatctatttccaaggagcccacttttaaaaatcttttcttgttttaggaaaggtttctaagtgagaggc
agcataacactaatagcacagagtctggggccagatatctgaagtgaaatctcagctctgccatgtcctagctttcatgatctttggc
aaattacctactctgtttgtgattcagtttcatgtctacttaaatgaataactgtatatacttaatatggctttgtgagaattagtaagtaaat
gtaaagcactcagaaccgtgtctggcataaggtaaataccatacaagcattagctattattagtagtattaaagataaaattttcact
gagaaatacaaagtaaaattttggactttatctttttaccaatagaacttgagatttataatgctatatgacttattttccaagattaaaa
gcttcattaggttgtttttggattcagatagagcataagcataatcatccaagctcctaggctacattaggtgtgtaaagctacctagta
gctgtgccagttaagagagaatgaacaaaatctggtgccagaaagagcttgtgccagggtgaatccaagcccagaaaataat
aggatttaaggggacacagatgcaatcccattgactcaaattctattaattcaagagaaatctgcttctaactacccttctgaaagat
gtaaaggagacagcttacagatgttactctagtttaatcagagccacataatgcaactccagcaacataaagatactagatgctgt
tttctgaagaaaatttctccacattgttcatgccaaaaacttaaacccgaatttgtagaatttgtagtggtgaattgaaagcgcaatag
atggacatatcaggggattggtattgtcttgacctacctttcccactaaagagtgttagaaagatgagattatgtgcataatttagggg
gtggtagaattcatggaaatctaagtttgaaaccaaaagtaatgataaactctattcatttgttcatttaaccctcattgcacatttaca
aaagattttagaaactaataaaaatatttgattccaaggatgctatgttaatgctataatgagaaagaaatgaaatctaattctggct
ctacctacttatgtggtcaaattctgagatttagtgtgcttatttataaagtggagatgatacttcactgcctacttcaaaagatgactgt
gagaagtaaatgggcctattttggagaaaattcttttaaattgtaatataccatagaaatatgaaatattatatataatatagaatcaa
gaggcctgtccaaaagtcctcccaaagtattataattttttatttcactgggacaaacatttttaaaatgcatcttaatgtagtgattgta
gaaaagtaaaaatttaagacatatttaaaaatgtgtcttgctcaaggctatattgagagccactactacatgattattgttacctagtgt
aaaatgttgggattgtgatagatggcatccaagagttccttctctctcaacattctgtgattcttaactcttagactatcaaatattataat
catagaatgtgatttttatgcttccacattctaactcatctggttctaatgattttctatgcagattggaaaagtaatcagcctacatctgta
ataggcatttagatgcagaaagtctaacattttgcaaagccaaattaagctaaaaccagtgagtcaactatcacttaacgctagtc
ataggtacttgagccctagtttttccagttttataatgtaaactctactggtccatctttacagtgacattgagaacagagagaatggta
aaaactacatactgctactccaaataaaataaattggaaattaatttctgattctgacctctatgtaaactgagctgatgataattatta
ttctagGCCACAGAACTGAAACATCTTCAGTGTCTAGAAGAAGAACTCAAACCTCTGGAGG
AAGTGCTAAATTTAGCTCAAAGCAAAAACTTTCACTTAAGACCCAGGGACTTAATCAGCA
ATATCAACGTAATAGTTCTGGAACTAAAGgtaaggcattactttatttgctctcctggaaataaaaaaaaaaaa
gtagggggaaaagtaccacattttaaagtgacataacatttttggtatttgtaaagtacccatgcatgtaattagcctacattttaagta
cactgtgaacatgaatcatttctaatgttaaatgattaactggggagtataagctactgagtttgcacctaccatctactaatggacaa
gcctcatcccaaactccatcacctttcatattaacacaaaactgggagtgagagaaggtactgagttgagtttcacagaaagcag
gcagattttactatatatttttcaattccttcagatcatttactggaatagccaatactgattacctgaaaggcttttcaaatggtgtttcctt
atcatttgatggaaggactacccataagagatttgtcttaaaaaaaaaaactggagccattaaaatggccagtggactaaacaa
acaacaatctttttagaggcaatccccactttcagaatcttaagtatttttaaatgcacaggaagcataaaatatgcaagggactca
ggtgatgtaaaagagattcacttttgtctttttatatcccgtctcctaaggtataaaattcatgagttaataggtatcctaaataagcagc
ataagtatagtagtaaaagacattcctaaaagtaactccagttgtgtccaaatgaatcacttattagtggactgtttcagttgaattaa
aaaaatacattgagatcaatgtcatctagacattgacagattcagttccttatctatggcaagagttttactctaaaataattaacatc
agaaaactcattcttaactcttgatacaaatttaagacaaaaccatgcaaaaatctgaaaactgtgtttcaaaagccaaacacttttt
aaaataaaaaaatcccaagatatgacaatatttaaacaattatgcttaagaggatacagaacactgcaacagttttttaaaagag
aatacttatttaaagggaacactctatctcacctgcttttgttcccagggtaggaatcacttcaaatttgaaaagctctcttttaaatctc
actatatatcaaaatatttcctccttagcttatcaactagaggaagcgtttaaatagctcctttcagcagagaagcctaatttctaaaa
agccagtccacagaacaaaatttctaatgtttaaacttttaaaagttggcaaattcacctgcattgatactatgatggggtagggata
ggtgtaagtatttatgaagatgttcttcacacaaatttatcccaaacagaagcatgtcctagcttactctagtgtagttctgttctgctttg
gggaaaatataaggagattcacttaagtagaaaaataggagactctaatcaagatttagaaaagaagaaagtataatgtgcata
tcaattcatacatttaacttacacaaatataggtgtacattcagaggaaaagcgatcaagtttatttcacatccagcatttaatatttgtc
tagatctatttttatttaaatctttatttgcacccaatttagggaaaaaatttttgtgttcattgactgaattaacaaatgaggaaaatctca
gcttctgtgttactatcatttggtatcataacaaaatatgtaattttggcattcattttgatcatttcaagaaaatgtgaataattaatatgttt
ggtaagcttgaaaataaaggcaacaggcctataagacttcaattgggaataactgtatataaggtaaactactctgtactttaaaa
aattaacatttttcttttatagGGATCTGAAACAACATTCATGTGTGAATATGCTGATGAGACAGCAA
CCATTGTAGAATTTCTGAACAGATGGATTACCTTTTGTCAAAGCATCATCTCAACACTGA
CTTGATAATTAAGTGCTTCCCACTTAAAACATATCAGGCCTTCTATTTATTTAAATATTTA
AATTTTATATTTATTGTTGAATGTATGGTTTGCTACCTATTGTAACTATTATTCTTAATCTT
AAAACTATAAATATGGATCTTTTATGATTCTTTTTGTAAGCCCTAGGGGCTCTAAAATGGT
TTCACTTATTTATCCCAAAATATTTATTATTATGTTGAATGTTAAATATAGTATCTATGTAG
ATTGGTTAGTAAAACTATTTAATAAATTTGAtaaatataaacaagcctggatatttgttattttggaaacagcac
agagtaagcatttaaatatttcttagttacttgtgtgaactgtaggatggttaaaatgcttacaaaagtcactctttctctgaagaaatat
gtagaacagagatgtagacttctcaaaagcccttgctttgtcctttcaagggctgatcagacccttagttctggcatctcttagcagatt
atattttccttcttcttaaaatgccaaacacaaacactcttgaaactcttcatagatttggtgtggctatgaattctccaatatcttacacc
ctgcccagtgctgtgaggaggctcacctgtatggcctatatcaaaggtcttccctgccctttggctttccattgggtcctgccactggg
gagtgctggtaggaactatgaggaacataagagattcccttgactccctccttgtggagtagacccaggatggctgtgtctctcaa
gcaaggaacccagattacctcaaggtggcactctgggtactttttccttctgagtgattctggtaatcttcccttgtccctttaagcctag
ggagggtggtacttttgctgttagcaactccagggtacttgtaccatcccttgcagtttccctgaactctgaccatagctttttaaatagt
ccttttattaaatcctccttttgattgagtatgccatctatttcctgctgggactcagatacagtaattgtatcagaaatagccccagaaa
atagaccctcaaaataggattctgggactgggttgttcatatattcaaggaatgcaaggataataggacatgggaaatctacgga
atgtagtagcatcgcaattactgaacttatcatcaatggtagaatgggatgaaatgcagacagatggcaagatgttgtgaggtcaa
atggctgtggcacttagttgctacagaaacaacagttataaaaattatgattattacctagattcttttgatgatgatgaccccagaca
gagaacaaaggaaaaaaaaagttatcaacatacaattaaaaacatacatgggcaaccagaatgcctcttcagcagctttgaa
gaagtcgttcctctcttcaaaattgccaaggagtagaagtaaaagggacccttctcattaaatacctcacttggaagatttttttttcac
atctcatccactaaatcttatcttggtcagttttaaggtcttagtgctcaatgaggcattcttctaccaggtgccttgacttctaccagaga
actgatgaaatggctgagactaccttttggccatttaggggttcttcatatagctgaaccaacaagcacgtaaaggaccaccgtac
tgagcagggtgactgattttgatgaaaagggggaaactcagtgccttctatagaaccgggcaacgactacacaataggtaatac
tatatttggaactcaggaaattcagtggggaatctcttagctttctatccttagtagtattggtcaatggaaaactgtaacaatccttaa
aattcaagaacatcaaagactcagatttcacagaagtgagatatagagtataccaccaggtaaataatgccacccaaccaaag
taatggcagagggtaaaggggaacacgcaaagggtagtggaagaatgcagctgtactaaccaattttcatgactagctattgag
gcagagaacagacttgagcagttttgtttttctccattttttatactttactatgtcaagttggacttgacatcgtctcttattctttatgtgaag
aacactggtgatacctaacattttagatttcaggaaaagtattgctgaattgacattaccctataatgatacaataactgatgggattt
catgtgtctcctttgctaggaacacaaaccttcttcccaaaggaaggaagagagcacatgctgaggaatgaaggtgtgaaccgt
gtatcttctactttt
TRL7 genomic sequence

SEQ ID NO: 6

ggtcttaccccagtcagacccaacacctcacttttataacaaatatttggtaatgcaccctttgttatatgaaaggagatatttgtggat
aatgtaaccccagtcttcatgataaacaaaaaggcccagctgatttccaaaatgcaccccagtttagaatcagtctggcaagtatc
acatgaaatcctattggtatttgattgggatcacacagcatttgcaaatcaatttaagcaacatttctatttttacaatattgtggcctcta
gccccaataacaattatttctcttcatttacttatatctttgtgtctgggcagggtcctgccaggaaacagacggcatgttaaagtgag
aaactgatgagttcagcaaagtgactatttatatatttggacaggatttaaggaagtaagaaaggatgatgcaacacttcagagg
ggagtctttccgacctcaggctgaaggagaaggaacgattactggaattcagggaggagagcatcaccaaacaagagcttcat
tagaggactgcagccaacgcagggccaggcggagggagccagagggaggcaggctctgctctccctcttcctgcccttcagttt
ccaaccacggcctcctattggccaaacccaaccagaagccagcgagcaaggggctactgatgaagcacatatagctcagcct
ccagagacacagaacaggataaaaggctgagacagtgggtctggtggggcaaagagaaagcttgcacctgccaggtaaag
cattatagtccccatcctccccccaccaccttagttcttgtgcatttcccatcagttttcttccaaagcatttcagatctcactgatttgaat
gggacctcttcttctattaccaatttggatcagtaattatttatgtatgagaaaactattgatttttacatatcgttgcatagcagagtggttt
aataaggagacatttggtttttactgcctgtgtgggtatcacttgctatgtgacttgaggcaaatccaatatttcttctgttataaattcca
gtatttgtaaaaaatgggtaataagatctctattttatatagttttagtatttaatgagataatacatataaagtcattaaaacagtgtctg
gctcataaaaaaccctcaataaatgtcacttattactgtatctggtttttgagctgctctattgcaccattgagttttcagcccagtatatg
ttaaccctgatcattatctgcagaagtccccgtgccacactctacatcatccaaattctctccaggtggactaagtagattaaagaa
ctttaaacataactaccatattttggctctatctacaaaatgtccaataatcagttaagaaaggaacaattctcttggggcccacactt
tgagaagcaaatgcagctgaacttttttagaggaaagtgagtgaaccaactggtagctttgccactgcttaaaaaccagcatccttt
ccagctgggtctaagacagaataaggtaaatttagatatgtctctaatatatctatagaacagtggttctcaacccggggtgtttttgc
cccttaggggataatttgcaatgtctggagacatctgtgattgtcataactggaagggggcagtgctattggcatctagtgggtatag
agcaagggtgctaccaaatatcctatggtgcaacagagaattatctggtcaaaaatgtaaatagtgctgagggtgagaaaccct
gctataaaaacgaaagaaatttggtctacagagttgtttggatttagacaagacgttgccccaatagtggtgatagaaataagag
gaaccccgtgcttttgcaaagcccatatctggggtggcttaaataatcatgctcctccccatcccccgacctgatctttgtagttggaa
actccagggctggctgcctgtagtctttgtgactacacttcctgcctcccatcacttcatctcagaagACTCCAGATATAGG
ATCACTCCATGCCATCAAGAAAGgtattttaaacattggaacacatatagataatttaagtaggtagatgtatgtgct
gttataaggaagtggggaggagagaagagggaaccgaaatcatatgcacaaaaattttttttagaatataaataaaaaatgtgg
tagtctaaaatgtcaattcttcaaagataaagttaggctttcagtaacgttagaaatggttttctggaatatgtctccagtctacctaact
ttgaggaagtaaatactgtaaatagatgtttcaaacgcattttaaagcaatgatcctagcatgtctttaagctacagtattgtgctgtctt
tgaaatgtaaactttgatgtcttctctttctcttagTTGATGCTATTGGGCCCATCTCAAGCTGATCTTGGCA
CCTCTCATGCTCTGCTCTCTTCAACCAGACCTCTACATTCCATTTTGGAAGAAGACTAAA
AATGgtaagaacagctcagagaaccttaaaaagtgttatctgtaatctttgtggaaacaactgaaaccagctggcaagagca
atattgaagaatctgtacttaggttatttgctgggggaaagtgcttcctgatatttcacaattggcattaatgaagggggcatgtcaca
atttcagattaatcaacgcttgctctgttcaacttcctacaagaattaaatatgtgctgtggggaggaggagcagatgtttgaattggg
gacatagcttctatgtatctcatttcttcagcctacaattttggctttaaagccataacaaatcactgaattactgaagttactttgtgctttt
tccagcatatggtgttgtcttaatgactgtgtggatgaaagtgtgtgggcaggctcatagcaataaaatacgggaaatccccgggc
ttgagtgctgtcaaagaaaactaaatttggacagtagataaagatactatcaggactattgcaatcggcagaaagagacctcagt
atagaaaggggctcaattccaaatacagccaaagaccagtaaagatttctggccaaggagtagagtgggggtcagtggatgg
aaaattactaagaggaaacatcaagggtaaaaggattctggctaaaccgacctgacaggattcttgctgaagacaggccagg
gtgatcagacctcacctgtggatggtgggagatgaggaatttgatcagatattgagggtgatcacataccaagaggagtggattat
caataaaatgacttagcaggattcctgcttgaactgggcaatgcaaagatggacatgaagccaaaggccgaagcctaggggtg
tagtagagcctgattaagttgaattaaggagagtctttgtcagcgctggctctcccagtcactagttgggggggccttgtgcctgtcat
caaagtcctctgaaactcaatttctctgactatgaaataggcattagaatccctcccctgttgccttccagggccactgtgaggctca
aataatagactatttttcaagtcctttgcaagtggtatgatgcaagtgtgagttattaggtatgccaaaacttagtcggaaaaagacgt
caagggcctttttctgaaattattttgtcacttaaatcagacacattctagatccgaatgttagctcctaggctcattttgtgtcaaagttct
aatgaagcattaaccatggggctattgttacaaaggaaacaactgcttacggtttcatttcctagaaacccagatgtctattttaatgc
aaacctatgcccacatctgtctttgccccttgatgggtggcataatgggaatgatagtaatacagagagctcacatttcttgaccact
caactatcatgctgagggctagatagacatgattctattttggcctcaaagtagccctataaggtagagataacgaaactggggctt
tgagaggttaaggagcttgggtggctctgaaagctgtgctgaagactcttctgttcttcctagaccaagcccagcacacacgcaat
aaagatgaggttggatatgatggcttcctactcaagtacaaaggggaaatagtatatcttttctaagaaaagacgtgaaaataattt
tcaatataagaaattcaaaaggcaaaaaagcacagggaaaatattcaactgtattgagtcatatggcagatcctttgatctagag
attacacttttagaaactcttcttaaagaagtgaccatgagactggataaaaaaatgtggcacatatacaccatggaatactatgc
agccataaaaaggaatgagatcatgtcctttgcagggacattgatgaagctggaagccattatcctcagcaaactaacacagga
acaaaaaaccaaacaccgcatgttctcacttataagtgggagctgaacagtgagaacacatggacacagggaggggaacaa
cactcactgaggcctgtaggaggagggtggggcaggagagagcattagggtaaaaagctaatgcatgctgggcttaataccta
ggtgatgggttgatctgtgcagcaaaccaccatggcacgtttaactatgtaacaaacctgcacatcctgcacatgtaccccagaa
cttaaaaaaacaagcaataaaataattttaaaaaaacaaaagaagtgatcgtggacatggaaaactatttaccaagatggtca
gtgcagccaggcaaaaaaaaaaaaaaaaaaaaaatcatgtcccatgttgggaaggggtgaattaattgtagtagactcattaa
atggaatattatgtaatcatcaaatcatgttttttaaaataatactgaatgacctaagaaagcactcatggtataatgttaaatgaaaa
aagcaagctagaaatggataagtaccgtgtattcctcatgtttttactgcacctgctaggcaaatactagatgctcactaaatgttgg
ataatctgtgatgatggtttacataaacacatgtgttgcatattctaatttcattcaacatccctactttataaccattttacagttggcaaa
tcagaggctcatgaggtcaagtgatttatgaaagtcagagagctcttacatgacagaacaaggacttaaaaccaaatttttgtact
gacaaagccttggctgttactagaatgcttctcaccatgtgaaatagatgcagggatgggaaattactattagaagggaccatctc
ccaaaatgtcaatagtggttcagcaaatttaaaagtaaaaatattattctgctcttaacctataggaaatttctttatggctaaaaaaa
ggttattaagtaatcaatttattaaattaatacaatctgattatttaaaaatttggaacgctgtactaaaattaaaaatcatcattacaga
ttaaccagccagtacctctgcaccccaagaataaataatgtatatccccgaaactcaccgaagtttagggctggggttggcaaac
tatggcccatgggctatatcccacctgctgtacagctcatgagctaaggggtttttttttaattgttgtttttaaaagactgaaaaatatca
gagcaaaattactattttgtgacatataaaagttacattcaagtttcagtgtttacaaatggttttattgtttgagtatttgtttacttattgttg
ataagtgcttttgcactacgatggcaaactattcaaggagttgggtagtgtgacagagaacctgatggcctgcaaagattaaacc
atttactaactggccctttacagaaaaagtacgtcaggccggggcttatagaaaacaaagggataaggtataaggtcaaatagg
tttgagagccctatggtctttggtgactgttgtgatgcataatagctgttgagttcctaatttatgtaagacaactttatatccttttattctttt
agtttgaaaactaagtctgttgggctaaaatgataggaagtaaatgataactctctcctttttttaaaaaaaagcaagtggtttacaac
cttgtacttaaacgttttggtgacataatgaaactgatattcatggtatttgtactttacagagattaaactaaaattaaaaatatttcaa
aattcacaaataggggatatttgttaataaatctatttgggaaattcctagcagaggctcagtctataaaatgaatagcatttcagca
acttcccttattcacagtgcttggttattctctagggagacatacacaacacatctctagttaccaaacaattcagtgtgatataaacat
ggcaaaaagtcaatgaatttgagggcaaggtttccagcaatcgccccggccattgcttacttcttccatgccctttctaagttttcttca
gccaggcagccatcccctctggtttctcccagacccccgctgcaggctccccgccatcacagaaagcccctcgctcacacgtctt
ggctcaagcaactctttgtcttagaaatgcagatcccaacatttccttttaaactcaggcaacttggcttttttctgctctgtgatcttgaa
agtcgcttggaggaacagctgagtgcatggggctgttgtcctctcagggctaacatgttgtagcccagggggtgcccaggggcct
ttctgactggttggttagttgggtaaaagagtagagtcaggagagcaggaaatcctttcttaactcactataaaaataaaagcgttc
cccaggcctcaaatagtctcatctcaagataaatttccttttgccaagattgctgctgaaaataatccattgtagccagataatagcta
tgcaaagaatatataatagactggcaggggcatgcctaccgattcaatacagaaaggtgagggtttcatttgctggggtgtagtgg
gtgggagaattccttattgcaatcacactctacttctccatccagaaaactctccaaccctcctggaggactctccattttctcctctttct
cctccttgtgtacctacctagaccatctgctcccatatgtcctgtctgacttcctgttccagttacctatcactgcgtaagagatcacctc
aaaatgcaatggcttcaaacaacaacaatcatatactgctttctatcatgggtccaggagttgactggactcattaggcagctctcc
cacagggtctctcttggggtggcagtcaggcggtgactgcgactggaatcacctgaagactcactctccaggtctgatgcctggg
ctaggagactcaacagctaggtgccgaagcagctgcagctcctcaagtgtctctgtctccatgtggtctctctaatatggtggttgtc
gtatagccaggcttcttacaagggtgatgactcaggactccaaagcaagtgggtgagagaaagggagagagggagaaacag
ggagagagagagagaaagtgtgtgtgtgccagtacgcgcgaggtgaaagctgtattgcctgtgaactacccaccatgtctttcgt
cctcttgacaggaaacctcctagaaatgtttgctgtctccaaatccctctccttacgttcttccaagaactttgaagtcatattttatgtag
ctactccttcaaaacatatctggtgttcggccagttcttacgccctccagcactgctacctgggacttctgcttgaatgactgtaatagc
ctctcaactagtctccctgctttcacccttgcccctcactgtctattctcaacacagcagccagcagcatccttctcaaatgtaagtca
gaccaactgattgtcagctcaaaaatttgcaatgcatctgcattccacccagagcagagaccgccatccatggaatggtagaga
aagcccaacatgctcagggacactccctctctgacttcatctcctattgttctcctacaccccctgcttcagcaatattggccccgttg
ccatttttgtgaatattctagcatgttttcaccttggggcctttgctccaggctaatccatctgtctggaatgcatttcccctggatgtctgtt
atggatgactttgtcctttccttgaggtctttgtttagatatcaacttcttaatgatgcctatccaagctgccctatttatcgtcacaatccta
ccccacattcctgatccttttcactctgccctgttttctttttcagtaacacttatcacttgacatgcaatatcatttctgacagttatatattttt
gtgattatttagagaacataagctatagttgagtggaaatcttttctattttgtccactgatgtcccaaacacctagagaagtacctggc
atgttgcaggcatcaataaatacttgttgaatttttcctttttcacaatttccttctacgttgttatgatgagatcttatttcctctgtaatttgattt
taaaagttttaataaaaaacaatacatattatttatgataaaaagtcaaagagtagagaagggtataacataaaaatagaagtcc
ccctcttcccagggaaggcccctttataccactgcccagaagaaattgctattaaaggtttcttgtgtattctttcctacttttctctgcaa
atacaaatatatgcatatatatttatcataaatgcattatatgttatatgttattttaatgctgctttaaaaatcccctttattttttgtaacttagt
agtagatcatgcatagctttttatgtcgatacccacagctctaccacattctttttaagggacatttgatattttactattggtagtttcccat
ttttaaccattctctcaaatcaatggattgtcatgtaattcttcctattcttactatttcagaaagctgaatcaaactagcaaaatagttttat
ctaaagacatataaggccgggcgtagtggctcttgcctgtaatcccagcactttgggaggctgaggcaggcagaccacctgaag
tcaggagtttgagaccagcctggccaacatggtgaaaccccgtctctgctaaaaatacaaaaattagctgggagtggtggcggc
tgtctgtaatcccagatactcaggaggctgaggcaggagaatcacttgaaccgggtaggcagaggttgcggtgatccaagatcg
ggccagtgtactccagcctgggcgacagagtgagactctgtctcaaaataaataaataaataaataataaagacatataatgctt
actttaaagaaaaacaaaacaaaacatgtactagttatttttttcctccctctgtggaattcttagaaggtttatggtagtttgaagctttg
catggaccattttgaaacagcagcagcctgaggttccagggggttatgaagactcccagctgaggacagaccctggcagataa
gtttcagggggctctacaccaaccattagagtcatagaataagcacaatagaaaaggaccattaaggtcagttagccaaactcc
agagtttgttgatgagaaagtcaaggttcaggataattcagttggtagccctgtagcagacagagagactgaaaacaaatctgac
tttcagttcacgtggtgctaacccctagaataaataaacacgaggagaaatcagactaatcccagtcttcttctaacttgtcacaag
acacaaaccacttaccttcacttcctcattttttccatctaatagttcccagttatatacatgtccttctcactcctctgattgcaaccagac
atctcttacaagtttacaaagttttgaagataaaaacgctatttggaaagcgtaaagttaaaaacagcttggtaaatgtttttttttttttct
attagtaattcgatctctacaactgtaaatattgtggtaggaatctaatacagatctaaaatcagtaaaattcaatcttgaatatgggct
tcagtcctgccatcaaaatagtgcatccaggtggataggttttgccaccttgaagagttgtttattcaaacttttgtttgaagagtagga
aagcagtgttacctttaggcctgacttagcccttgccccacaatctattgttttttctcaccatagatttccctgacagcagagagaga
gttctgtgctcaagagatacacacagcttctgacaatagagcagcagagtatttggttcctaattgagcaggaatggtgtttgactca
tcatcatttccctactttgtctagcacagtaccttgcacagagtagattctcaataatgtttgttgaatgactgtgggagcatataattcat
aatggagacaaagctcaatgaggctttaaatttctaaatccacaaaatgccctcatgtaacattgctggatgatatggtttagctgtg
tccccacctaaatctcaccttgaattgtagctcccataatccccacgtgttgtgggagggacccagtgggaggtaattgaatcatgg
gggcgggtttttcccatgctgttctcatgatagtggataagtctcacaagatctgatggtttcataaacggcagttcccctgcacatgct
ctcttgcctgacgccatgtaagacgtaattttgctcctccttcaccttccaccatgattgtgaggcctcctcagtcatgtggaactgtga
gtccattaaatctctttttctttataaattacccaaactcggatatgtttttattagcagcatgagaacagactaatacaatggacattgg
atgcaattcatttaaaaaatcatcttaaaaatatctttcttttttctccctcaagttggtcccactcaaaacataaacacaccatttttttttttt
tttgtcttgagacagagtcttgctctgtcacccaggctggagtgcagtggtatgatcgtggcttactgcaacctctgcctcccgagttc
aagcaattctcctgcgtcagcctcctgagtagctgggattacaggtgcatgccaccatgcccggctaattttgtatttttagtagaaat
agggtttcaccatgttggccatgctggtctcaaactcctcacctcaggtgatcctcccgccttggactcccaaagtgctgggatttcat
gtgtgagccagtgtgcccagccaccattttttaatacttgtaaatttttcctataaaaacaaaccaatttctctatgccccaaaaccgct
aagtagcacaaaatagaaacattagagtaccaagaatacttgaactgaaaaggaaattaatcaaaatgcagacacacattata
ccaagtgcatttgctgtagctgtgtaaggcaacttgaatagaattggtcaacaatgagtctgaatcttggtttgaaattgcctgtctgat
ctctgcttcctcatcagtaaaatgagaatatttatatggcctttcaacttcagtgtgagggatcaatgatgtaatataaacaacaagtct
gccttagaacctggcacaccataagtaataaaaggcagccaatattttaaaaaatacacaaatcatggtctgatggctgtccaat
ataaattctctattttccattttaactaaagagacgatatattgagaaaatagaaacacctgtgtgtatgaaatcacccattcccattttt
acaataattagtttgctaattgagcatccaaatttacccagtgtatttgcatgtgtaattagctgtgattcaataccaaagccaggccta
tcatggtatactatgctattttacaagtcaaattactgaaagatgcatgtctttaggcaatcattacaaataaaaaaaaaaaaaccg
aagcaaaacaaaataacatagattatttgtatcagatggacaaaacagacctggcttgatgccgaacccttaaatctcaaaataa
cgatagttgaagctaaggttccagcttaagtctgaagcaggtagtttccaatggcttgaaaggagaaatttctacactgaaggaaa
tttccattggaataaaggaatatttcacacttttaagtcatcttctctagatggtcttttgggtatactttctctttaaataacagatttagaa
gcactttgttcatttgtttagaattaattccattcacaagtttaacacagcctaaggtttggtctagaccaggggtctgccagctatgac
ctctgggctaaatctgtcccttcacctgctttttttttttttttttttccaacctgtgagctaagaatgggttttactattctaataaatagtgagtt
catttttctccctcacctgcttgatcagagcccaactttctcattgcagttaatcttccttctggcatggatcttggaatgcaaacttgctgg
gatctccgagttccaggcttcccgtgcagccggtgtggagagccaagagatgttttgtttggcataaagcattccaagggtcagtg
ggcttgggctcaactattgagcataggacaagggcagccccatcctgactgtgactcttcccacaagagacaaacgagctctgt
gctttcactggggtttcaggttcaaagggacagagcgtctgagaaaaaggattatgaaagagtccgtctgcagctccacttcccgt
gcccttccaatgataccatcctcgtttcttctgtggcatgctccccacttcaatccttccttcagaggccccaaaccctcctggtctctcc
ttgtcaccttgtgaaaatctgatcttcagggaaaaattccttactatttatactagtataatgtgaatcttctatgggattttaagaaagttc
aaagccttggtttactcagcaaatatttagcttgcactcactatgtggcgggcatcctaatgatggagtatatgtaaagacaaaaaa
agtttccggacctcaaagtgttctccatctataggggcagatgactgagttgacatctcgagaagtagaatagcagagtggctaag
agtgccagctctgtctcaatcacctaggtctcacctcagcattaatttcactttcctcattgtaaatgagcatatctcttagaattgggat
aagcattaaataatatagacttggaatgaatttgcttagaactaattccatgcacaagtttatcacagcctaaggtttggtctagacc
agaggtctgccaagtatgacctgtgggctcaatctgtcccactacctattgttgttgttgctgttgttttttaatgacctgtgagctaagaa
tgggttttactattctaattagttacattctcaatggttatttaagtacctccataatatcctcaattttgcctaaaatatttaccatctggccc
tttacagaataagtttgctgacttattggtctggaccaatgctatctaataaaactttctgcaatgatgaaaatggtctctatctgtaccct
tgaatacagcagccactagcctaatgtggctttttgagctcttgaaatatagttagtgtgactaagagattgaattttaattaatttaaatt
tatggagccacatgtgactatgacattagagcagctctagacagcctgaagtctaaagactctatgctttgtcggtgctcccctctct
caattgaatcaactaccctgaggctgcatgagtcaaggggaaggccacactcttcaatcagattttttgccctggactggctttcatt
gtctactagaaaatgcttaatgggaagtgcttagaaaatgtacatgggcatacacttaattaatctaagttgctgctttgtctgtatcca
ttaaatctgctttattttggggtaaactacagtagaagttggctttttcaaccctgcaaagccttaaaattcaggatgtcttactcaactta
aagtgtagagttgcagccagagcacaactgtatttccttctagccctgcttgcagaatggctaacttcagtcctatttcatttctcttgta
agactgctaaaaacagtaagaagccaccaacatcattatgaatattgccaaatcatttcgcctaagagtaaagtcacagttggca
tgtgttctgccctccaagacaagatagcataggtgacagttttatcagatatcttgtgatggcataatataggccacccagctttcca
gcctctgatatctgagtcttcccaatagcctgatgacatccgcatcacatattttaggttcgctcatggacagtaacttatttccaaattc
tatactggttaaaattaggtttgcatttgtgcaatagaaaatccaattgacattggcttagcataacaattttttgatttctcataaactctt
ggcagtcagcaggtccaagccattatttctgctctgctctctgaggtcatataaggaaggatctggatgctctgggtcatctacgtcat
ctaactggttgctgtgccatccctagctcatttttctcatgtgcattgcccaagatggctggctacaacatccacattacaagaagcca
ggtggaagcagacaggagaaagaggagaaaggggaactgccccaccgtttaaggacatgtcccagaaactgtacacctca
cttcctcccaaatttcactggctatcacttagtcatatagccacacttagctgcaagtgtgtctgggagatataattatttttcacagtggt
atatgcccaactacaaatggaggttctgtcattatgagatgagagaaaggcagaaaacatgttgagagatgtgtagcaatctctg
gactccacggggataaaaaagaattgagagtatcaaaattcaggatcaaaatcaaaattaaagataaaaaatatcaataacta
tcacctggaataagaacaacgtacagttcagctacacatatacaagtggcagcatcttgtctggaaggaactaatggtctttctac
attgtattttagatatgtatttttttttctcccttccacaggattttgagctccttaagggcagagactttgtgtctcctgctcctagtaggcat
ccaacacgtatctgtcaactgaaagaatgaatatgagtcagtagatacatattagaattctaatatccactggctgggtccttggtgt
gtcccatattgttgtttctgtgtccatcattcttttgcagggtatcttctactgggcacagaacctgcctcagaggggcatatgggtaatg
aactaccaagaaaggagtagaacccagttcttccaaccctccacccagagtgcttttcacaacctcatgtgtaataagtgcagta
ggagatgagaggagggagtgattacttctgtctggtttgatcccagaaggttttttgaagaaagtgtttttgaatgagacattatgaaa
acagagcttcttaaaccttttcccccaaggaatccctgggcagatagaagagacagaaatctgacctctgcttagtctgggggtat
agactgaaggaacctactcaaaggagaaatttttctcatttttctttacttcacgattcatatatgcaggcattcattctttcattcatgtat
ctcacagacataacgaggtcctaattaagtgccaggcattgttttacatgagaccacaagaggccctaccctcttgcagcttacatt
cttgtacagaatagacatcatacgaataagcaacataaatcatcaagataatttctgaccgtggtaagggctatgaccgaaatca
aacagggtagtcagttacagagtgcatatacctctctgtgcctcagttgactcatctgtaaaatggagataataatagaggtctagg
ctaggcatggtggctcatgcctgtaatcccagcactttgggaggccgaggtgggtggttcacttggggtcaggcattccagaccag
cctaaccaacatggtgaaaccccgtctctactaaaaatacaaaaattagccaggcctggtggtgcatacctgtaatcccagctac
ttgggaggctgaagcaggagaatcgcttgaacccgggaggtggaggttgcagtgaaccgagattatgccattgcactccagcct
gggcaataagagcgaaactcagtctcaaataataataataataataataataataataatagtctataattccaaaacccaaaac
tgaaagctttgtcctaactcagttgattgcaaacataatatgatctgaatgcatttggaggtagatcttgacctgaactgaagttatttat
tctttttaataaataaatgagttatttattctttttaataaataaatgagtcatttattctttttaataaatgagttattctttttaataaataataa
actgagttatttattctttttaataaataataaataactgagttatttattctttttaataaataataaatgagttatttattctttttaataaataa
taaataactgagttatttattcttttttttttaataattccacttagagtggacaatcctatatgtcactgcagaaattttgtgtgtttgattatgg
aatgctgccccaggcctcaatagttattacataatttagggtacatgtagcgtattaccttctaaaatttgaaaaattccgaattccaa
aacacatgtagcaccaaaggtttcggataagggattgaagacctgtagtatccattattgtgaggattaaatgaatgaatatatgg
aaaacacttaaaatgatgcctggcatgtggtaagtgctacgtaagttaactactattactattattatcactattcttacatgagaagat
atttagataagttggtcagggaaagcctctctgaggatgtgtcacttgaataggcaactaaggggtggtaatgaccgggctgtggg
aagaggaggagaaagatgatttcaggaataggaaacagcaagtgccaagactgtggtggttacaaggctggcttgaatgcag
aacagaaaacagaccagatggctgatatgtggtaaaggaggggaaagatggctcaaggtcagagaggtaggctgaagtca
gaacacccttgatataagcaatggtagagactttggatttcatttaaagtgtaataggaagacattatagttgatctgattcaggtttat
aaagaacgctctgatgctgttggatgaatgaattatagaggagaagggggagcagggagagcaatttggagtctagcatagtg
gtccagatgagacctaatgactaattggagttgggaggtggtaatagtcaaagagaaaagtggacaggtgcgagaaaaaagtt
tagaaataagtggggggcgggggaggttttctgattaatttgcattctaatttataatatgtcactgtgtagaggctaaaaatttcaca
gtcattgtctcaggtgtgttaaggccagtggcgtgctggaccccacttgaaattggccatggagggaatatttacactatagaaattg
acaaatgctacaaatcaagacaacaaatcaggcaaagcttcttgttaaacatttaccatcacaccactggtgaaggtgacttgatt
tttccacaactaaacttccttcatttcacagcctccattttccctgatcacgaaaacacttaaactaggcacatcctcggaaacgcag
tatgaggactgctgtgtcaatcacttcatgtttttaactcaattcagcgatcctcccacttcttcccaggctctcatttaggtacatggga
atgggatgggaagagggacctggttcatgattgtcatttacccaccttggccccctctgaagtacaactccactctctgctttacaat
atcactctgggcagcattaccaattgcctcctgatagtgggatctatgaacccattatgtctttggacaaaagcatagccaggggtt
gggtccagggcctgggatcctataaccgtacaaatcctattatcagggactataaaatcctattatcagggaccatagccatccct
ctatcttgactcaactcctcctccctgagtagtgaacatttttcctaaatctctgagaaagactggtgctctagaaagatgtaccatattt
atttaagggcttcctgtacccactggcatattgccatatattctgaggtatctgagtgctccttttgagaaacatagccttaaaggataa
gtagaaatctggtgggtgaaaatggtagggaagaggacttctaacggagggacttgcaagtcagggaacttgggtttatcgact
agtgaggctagtagaggaattcaatcaggtaagccggacaagtagacagggtacaaattatggaagactttggatgccatgata
aaaagcttcagctcatactgtaaaaaataaaataaaataagaaggttgggtgcagtggctcatgactgtaatttcagcactttggg
aggctgaggtgggacgatcgcttgagcctgggaaacaatttcaaggagttcacagcaagaaactgactgattaaggtttgggaa
gcttgatagatagggtagactgggaaagtgagagaggaggctttggagtggaccaaggatagagggatctcagctgatattatg
tcagctaaaacctcaaagcaaggaggatgttaagaacaatgaaggaggtcagctggactctcaatgtttttaacgatagggagg
aaaagataggggggtgacaagaagaagagacaattttgtacctctaactccaacaaactttagacctgaaaaatcccttctgag
ccatcttgcattggagaaaaaaaattgcttatttacctccaattagaggaattaagggaagtaggatttttttgtttttcttttgagacagg
gtcttgctctgtcaccctggctggggtgcagtggtgtgatcacggctcactgcaacctcaaactcttgggcttaagaggtcctcccaa
ctcaacctcccgagtagctgaactacagttgtgtgccaccatgcccagctaattttttattttctgtagagagaggggtctcacgctat
gttgcccaggctagtcttgaactctggcctcaagcgatccgcctgccttgtcctcccaaagcgttggtattagaggcatgagccacc
acatctggtggaagtaggcatttggtttcttagataacaacatgattggttgattcagtcacttgggaagataaaagcattaactgag
ctagatccctatggtagagacacaggctggaccactccatgcgtaagtactaaactaaaaccagtgttctggagtagacattgct
agaaatcctgaaacttgagagccagtccacggttaaagcattctgtaaggcagagccagtggaaggtaataaggtgatttttaaa
gctcttctgcacttcccatattcccttttagggcctttctccctagggtcccagtgtctgtcatgctaaacctagatgcacaacaatcatc
tttatgggtagtttcccatatgtcccagtttgcctgacagactcttggtttatgcctatagtcttggtgtaattattaccagccccacttcatt
cttgtaagtatactaatggatcagttatacggttcctctgattatgtatcacctaggcagtgccctgactctactactatctcctctccaa
atttatgtaatgtaaacccaatgtgtagggaaaatgctcatcctaaaatctccttggaggggataatttgcaagattctttgcaaaaa
caatccaagacaagagccagattatggaatgtcagtgccagaatggcaggaatgtatgttttctaatcaaatgccacttactactg
ggtaaccttgggctaatcagttaatattgctgagcgatgtcttcatttgtaaaacgggaatcttagaatattctgagactcaaatactat
gaaagactcatgtaatgtgtaccagggcaggtttagcaggccgacataaattgcactaaagtcttcatgtgttatttttcatgggtgta
tccatattctaacatttcttcaccctccaaatttcagactttggcagtgaatctatggctctgcaattttagtgttccatgtaacaacgaat
aggaaaatgctgcttctaccctctcgaaagctattttgctaaagagctaagatgctaaaagctaaatatgtaactaaatagttgcaa
atctcagtaactgacaaatacagtcatggggttggggatgctgtttagacagctgaaaataagacctgaattgtttatttttaaaatgtt
gcaaaagagaggcagcaaatgggaatttttaattctgattcttggtatgttttagaacaatgatttgttctttcttatactttcagGTGT
TTCCAATGTGGACACTGAAGAGACAAATTCTTATCCTTTTTAACATAATCCTAATTTCCAA
ACTCCTTGGGGCTAGATGGTTTCCTAAAACTCTGCCCTGTGATGTCACTCTGGATGTTC
CAAAGAACCATGTGATCGTGGACTGCACAGACAAGCATTTGACAGAAATTCCTGGAGGT
ATTCCCACGAACACCACGAACCTCACCCTCACCATTAACCACATACCAGACATCTCCCC
AGCGTCCTTTCACAGACTGGACCATCTGGTAGAGATCGATTTCAGATGCAACTGTGTAC
CTATTCCACTGGGGTCAAAAAACAACATGTGCATCAAGAGGCTGCAGATTAAACCCAGA
AGCTTTAGTGGACTCACTTATTTAAAATCCCTTTACCTGGATGGAAACCAGCTACTAGAG
ATACCGCAGGGCCTCCCGCCTAGCTTACAGCTTCTCAGCCTTGAGGCCAACAACATCTT
TTCCATCAGAAAAGAGAATCTAACAGAACTGGCCAACATAGAAATACTCTACCTGGGCC
AAAACTGTTATTATCGAAATCCTTGTTATGTTTCATATTCAATAGAGAAAGATGCCTTCCT
AAACTTGACAAAGTTAAAAGTGCTCTCCCTGAAAGATAACAATGTCACAGCCGTCCCTA
CTGTTTTGCCATCTACTTTAACAGAACTATATCTCTACAACAACATGATTGCAAAAATCCA
AGAAGATGATTTTAATAACCTCAACCAATTACAAATTCTTGACCTAAGTGGAAATTGCCC
TCGTTGTTATAATGCCCCATTTCCTTGTGCGCCGTGTAAAAATAATTCTCCCCTACAGAT
CCCTGTAAATGCTTTTGATGCGCTGACAGAATTAAAAGTTTTACGTCTACACAGTAACTC
TCTTCAGCATGTGCCCCCAAGATGGTTTAAGAACATCAACAAACTCCAGGAACTGGATC
TGTCCCAAAACTTCTTGGCCAAAGAAATTGGGGATGCTAAATTTCTGCATTTTCTCCCCA
GCCTCATCCAATTGGATCTGTCTTTCAATTTTGAACTTCAGGTCTATCGTGCATCTATGA
ATCTATCACAAGCATTTTCTTCACTGAAAAGCCTGAAAATTCTGCGGATCAGAGGATATG
TCTTTAAAGAGTTGAAAAGCTTTAACCTCTCGCCATTACATAATCTTCAAAATCTTGAAGT
TCTTGATCTTGGCACTAACTTTATAAAAATTGCTAACCTCAGCATGTTTAAACAATTTAAA
AGACTGAAAGTCATAGATCTTTCAGTGAATAAAATATCACCTTCAGGAGATTCAAGTGAA
GTTGGCTTCTGCTCAAATGCCAGAACTTCTGTAGAAAGTTATGAACCCCAGGTCCTGGA
ACAATTACATTATTTCAGATATGATAAGTATGCAAGGAGTTGCAGATTCAAAAACAAAGA
GGCTTCTTTCATGTCTGTTAATGAAAGCTGCTACAAGTATGGGCAGACCTTGGATCTAA
GTAAAAATAGTATATTTTTTGTCAAGTCCTCTGATTTTCAGCATCTTTCTTTCCTCAAATG
CCTGAATCTGTCAGGAAATCTCATTAGCCAAACTCTTAATGGCAGTGAATTCCAACCTTT
AGCAGAGCTGAGATATTTGGACTTCTCCAACAACCGGCTTGATTTACTCCATTCAACAG
CATTTGAAGAGCTTCACAAACTGGAAGTTCTGGATATAAGCAGTAATAGCCATTATTTTC
AATCAGAAGGAATTACTCATATGCTAAACTTTACCAAGAACCTAAAGGTTCTGCAGAAAC
TGATGATGAACGACAATGACATCTCTTCCTCCACCAGCAGGACCATGGAGAGTGAGTCT
CTTAGAACTCTGGAATTCAGAGGAAATCACTTAGATGTTTTATGGAGAGAAGGTGATAA
CAGATACTTACAATTATTCAAGAATCTGCTAAAATTAGAGGAATTAGACATCTCTAAAAAT
TCCCTAAGTTTCTTGCCTTCTGGAGTTTTTGATGGTATGCCTCCAAATCTAAAGAATCTC
TCTTTGGCCAAAAATGGGCTCAAATCTTTCAGTTGGAAGAAACTCCAGTGTCTAAAGAA
CCTGGAAACTTTGGACCTCAGCCACAACCAACTGACCACTGTCCCTGAGAGATTATCCA
ACTGTTCCAGAAGCCTCAAGAATCTGATTCTTAAGAATAATCAAATCAGGAGTCTGACGA
AGTATTTTCTACAAGATGCCTTCCAGTTGCGATATCTGGATCTCAGCTCAAATAAAATCC
AGATGATCCAAAAGACCAGCTTCCCAGAAAATGTCCTCAACAATCTGAAGATGTTGCTTT
TGCATCATAATCGGTTTCTGTGCACCTGTGATGCTGTGTGGTTTGTCTGGTGGGTTAAC
CATACGGAGGTGACTATTCCTTACCTGGCCACAGATGTGACTTGTGTGGGGCCAGGAG
CACACAAGGGCCAAAGTGTGATCTCCCTGGATCTGTACACCTGTGAGTTAGATCTGACT
AACCTGATTCTGTTCTCACTTTCCATATCTGTATCTCTCTTTCTCATGGTGATGATGACA
GCAAGTCACCTCTATTTCTGGGATGTGTGGTATATTTACCATTTCTGTAAGGCCAAGATA
AAGGGGTATCAGCGTCTAATATCACCAGACTGTTGCTATGATGCTTTTATTGTGTATGAC
ACTAAAGACCCAGCTGTGACCGAGTGGGTTTTGGCTGAGCTGGTGGCCAAACTGGAAG
ACCCAAGAGAGAAACATTTTAATTTATGTCTCGAGGAAAGGGACTGGTTACCAGGGCAG
CCAGTTCTGGAAAACCTTTCCCAGAGCATACAGCTTAGCAAAAAGACAGTGTTTGTGAT
GACAGACAAGTATGCAAAGACTGAAAATTTTAAGATAGCATTTTACTTGTCCCATCAGAG
GCTCATGGATGAAAAAGTTGATGTGATTATCTTGATATTTCTTGAGAAGCCCTTTCAGAA
GTCCAAGTTCCTCCAGCTCCGGAAAAGGCTCTGTGGGAGTTCTGTCCTTGAGTGGCCA
ACAAACCCGCAAGCTCACCCATACTTCTGGCAGTGTCTAAAGAACGCCCTGGCCACAG
ACAATCATGTGGCCTATAGTCAGGTGTTCAAGGAAACGGTCTAGCCCTTCTTTGCAAAA
CACAACTGCCTAGTTTACCAAGGAGAGGCCTGGCTGTTTAAATTGTTTTCATATATATCA
CACCAAAAGCGTGTTTTGAAATTCTTCAAGAAATGAGATTGCCCATATTTCAGGGGAGC
CACCAACGTCTGTCACAGGAGTTGGAAAGATGGGGTTTATATAATGCATCAAGTCTTCT
TTCTTATCTCTCTGTGTCTCTATTTGCACTTGAGTCTCTCACCTCAGCTCCTGTAAAAGA
GTGGCAAGTAAAAAACATGGGGCTCTGATTCTCCTGTAATTGTGATAATTAAATATACAC
ACAATCATGACATTGAGAAGAACTGCATTTCTACCCTTAAAAAGTACTGGTATATACAGA
AATAGGGTTAAAAAAAACTCAAGCTCTCTCTATATGAGACCAAAATGTACTAGAGTTAGT
TTAGTGAAATAAAAAACCAGTCAGCTGGCCGGGCATGGTGGCTCATGCTTGTAATCCCA
GCACTTTGGGAGGCCGAGGCAGGTGGATCACGAGGTCAGGAGTTTGAGACCAGTCTG
GCCAACATGGTGAAACCCCGTCTGTACTAAAAATACAAAAATTAGCTGGGCGTGGTGGT
GGGTGCCTGTAATCCCAGCTACTTGGGAGGCTGAGGCAGGAGAATCGCTTGAACCCG
GGAGGTGGAGGTGGCAGTGAGCCGAGATCACGCCACTGCAATGCAGCCCGGGCAACA
GAGCTAGACTGTCTCAAAAGAACAAAAAAAAAAAAACACAAAAAAACTCAGTCAGCTTCT
TAACCAATTGCTTCCGTGTCATCCAGGGCCCCATTCTGTGCAGATTGAGTGTGGGCACC
ACACAGGTGGTTGCTGCTTCAGTGCTTCCTGCTCTTTTTCCTTGGGCCTGCTTCTGGGT
TCCATAGGGAAACAGTAAGAAAGAAAGACACATCCTTACCATAAATGCATATGGTCCAC
CTACAAATAGAAAAATATTTAAATGATCTGCCTTTATACAAAGTGATATTCTCTACCTTTG
ATAATTTACCTGCTTAAATGTTTTTATCTGCACTGCAAAGTACTGTATCCAAAGTAAAATT
TCCTCATCCAATATCTTTCAAACTGTTTTGTTAACTAATGCCATATATTTGTAAGTATCTG
CACACTTGATACAGCAACGTTAGATGGTTTTGATGGTAAACCCTAAAGGAGGACTCCAA
GAGTGTGTATTTATTTATAGTTTTATCAGAGATGACAATTATTTGAATGCCAATTATATGG
ATTCCTTTCATTTTTTGCTGGAGGATGGGAGAAGAAACCAAAGTTTATAGACCTTCACAT
TGAGAAAGCTTCAGTTTTGAACTTCAGCTATCAGATTCAAAAACAACAGAAAGAACCAAG
ACATTCTTAAGATGCCTGTACTTTCAGCTGGGTATAAATTCATGAGTTCAAAGATTGAAA
CCTGACCAATTTGCTTTATTTCATGGAAGAAGTGATCTACAAAGGTGTTTGTGCCATTTG
GAAAACAGCGTGCATGTGTTCAAGCCTTAGATTGGCGATGTCGTATTTTCCTCACGTGT
GGCAATGCCAAAGGCTTTACTTTACCTGTGAGTACACACTATATGAATTATTTCCAACGT
ACATTTAATCAATAAGGGTCACAAATTCCCAAATCAATCTCTGGAATAAATAGAGAGGTA
ATTAAATTGCTGGAGCCAACTATTTCACAACTTCTGTAAGCtttattgtgtttcatagtttccgttcttcttctgt
gagaacaaggataatggcattaaaaaatcagcttttggtcattataaattgtcttctattaaaacacatatacacataaaatcacttg
aagacaatttaaacatcttctgaaatggatcaagaggaagggaaactgaaaataatgcaactcagaaaccacagagtattttga
catgaggttaagcaccgtggtttgttgtaggaaaataacagcacaccaacagatggtttttatctgaattctttggtaatcttgacatgt
cattcttctaactttctgagggccctcagtgcagttttgtaggactggagctgttcacagacggtccccacaaagctctgaacgtggg
gcttctctgctgactggcctctggttggctccaccccggaaggaactcccagattctccatgaattccgcttccaccatcaagccttg
gtccaagcccctttcaaccttgacttggccaggaagtgtcctttctcttcagatagatactacaccttagcaagacttggcatttttaga
atccaagccaagggaggcacttggcaaggcaaatgttatggatgagaaaaaggcaaaacaagtgtctgcagtttgtagagga
gagagaggatgagtctgattgtagccctgaccctgagtcaggatctctcggccccatttgcaggtctacttccagctccatctgtctg
gacactcttttaggtccagatcatctcttacatgtggccaaggaatatagagtatgcaaggggatgtagcgacctgagagtgtgag
taacttgtgcccatctccaaggaagctgtgatggggatagcaaggacacacactcttcttatttataatgcctttccccctcccatga
gatatgctttttatttacttcctccttctcatcctaagtcgggtgaacaagaggaccaggttgcacatcctactacttatttatggcccaat
tttaacatgggggtggagttgaggttggaattgttcctccgcctctgctgcacatgctcagtaagcaagaacactgttgatgggaaa
ggcttagtcacagacagtgggagcacatccctccttggagctttgggtcgctgtgctccagaaacagttagttatagcacaccctg
ctcctggcatctactggaaggtgaagcccttgaccctaagaaacattgggaatgatttgtaccctccaaagtccaatagctatgtcg
gagggaaacgatcaaagaacatgattgaggagactcaaacagagatgtgcttcagacaacaccaagacagaaaattaatcc
attttaccaagttaacaatgtactgaaggcgaacaagagaccaacccacctgccaaccaacgctatgaagaaggagggttgat
cagtctggctaacatggtgaaaccccatctctactaaatatacaaaattagctgggcgtggtggcacactcctgtaatcccagcta
ctcgggaggctgaggcaggagaatcgcttgaacctgggaggcagaggttgcagtgagctaggatcaagccactgcactccag
cctgggtgagagagtgagacttggtctccaaaaaaaaaaaaaaaagaaggagggttaaaaagagaataagtcccaaactca
taagatggtgtggaaagggccctggtgacataggggccacccatgccagtgagaatgaaatcacaacagggcagtttcacact
gtttcaggtttttattttttcttcttcttctttccctcctttcttcttttgcctccccctccctctcggtttcctttttggctctagacacccacagcaa
gtgtcaagcaatgtacaagaatgaaaagaagacagccgttgttgcaggtggatgcttctgtttggaaggtgtggttttgtgtgcacttt
tggttggaaacctatgtctctctcacacacatgtcccccacctgcttcagtgagca
TRL8 (isoform 1) genomic sequence

SEQ ID NO: 7

atatatatcatatatacatgatgatacacacacacacacacacacacacacacacacatatatatatatacgtatacaagcatgct
ttacaaggccaattgactggtctacaattggctgacacttggtggcctagaagccagggtatgtgagtctcgcttttctagaaagctg
acaaactctccagttccaaggatccttgctcagtcaacggctggaagtcatttttacttcgctgttttttgtttgtttgtttgtttgtttttttaga
caaagtctcattctgtcacccaggctagagtgcagtggcactatcatggctcactgcaatctccacctcctgggctcaagcgatcct
cccacctcagccacccgagtaactgggactacaggtgcacaccaccatgcctggctaatttttgtatttttttagagacaaggttttg
ccatgttgcccgggttggtctcaaacccctgagcacaagtgatcctcctgcctcggcctctacaaagtgctggaattacaggtgtga
gccactgcactcgatccattcttacttactttctttactttatttccaagcaaatgtttggagggaaaccaagagacttggatgcggcca
gccgaggcctttgggtttacaatcacaaatgtttttggtttgcccatgaaggcccaggctgcactctctgatgtcacaggaatcacct
ctcaaaccatgcaccaggtcttgaattcccttagggtgtgatctttagaggtccatctaggtatacccacccaagccattctttgactg
ctgacaggccttccttcataacaaggtgttccacagtccatttatatatggatgtcatctctgcccaccctgctgccaatttggttttctcc
cactcctggggtgtaaggcaagatgaaacatatcacatcccgttctaaactttattcttgtggccaggggtcagcaaactttttctgta
aagggccagatggcaaatatcttaggttttacaggccaagaagcaaatttggcatattatgtagctacttatatagtaaaataaaaa
tttccacaattatgtaattgatgaaactcaaaatgtaataataataatcgaaggcagtttttttgtagtataggtttaataatgagaaga
atggaatcatttttggaggtgctaacattctgcttggttggaatttaaagttagtgttctgtatcagcaaatccattgccaatgttcatcta
aaaatgttttcacttctgggccggatttcgttcaaaggctgcagtttgctgacctctgctcttggttacaccttttgaggcccttgctctcc
gagcataaaatggaatccatttatcagactaaatcgggaagattaaattttccagcctcacgaatgctcagccattgactcactcgt
tcatacaatgaacactcattgagcttatactacatgccaggtgctggaggaggcatggggcgcccaggagaaagatgctcgcttt
gcggccacagcccagtgggagggagacccatacctaccggtgctgtctcagaaacttgtggaacaaagatgaagcaatgttca
tgttattcgcctacatctgtgaattacacaaggaagacgagtttgagaaatccgaagttcagtacaaatttatggtaacttttttaaaa
aagaatacactgaagttttcttagtgaatggaataatgttccctttttctcccctgtacacacaaatacacaaaaactaacaaaaata
cgtcgtgtgtgtctgatttgggttgtatttaaatcatttcataaatgactttttcccataacttcagtttcaaagttttaaagcacagtcaatt
aatgatttggcaacagctaagaaatcacaagttcccttcttttcatgtaaacttctgtaaaacacacgctacgttctgctgatggtaaa
tagagccatttcaggaagttagccagtttctcttctcggccacCTCCTGCATAGAGGGTACCATTCTGCGCTG
CTGCAAGTTACGGAATGAAAAATTAGAACAACAGAAACATGgtaagccacttctatttctttagcaaag
ctttccaacagaatatggggtttctgacccagaaatctgggttggtggcaaatggtgtgagcctagaaagtaataaatgggcaaat
aaggataaaaattaaagatcgaaacaactgtaaatgcaggtaaagcggcttgctatgatctttaatttgtgcacacgttagtataaa
ggaattagagagtaaattttgaaaatcaaatgcagtgatgatcttactaatttggacaggaaaataagaaaatttcaagttagaaat
tgaactggaaatattacttactggccctaccagagacaatatcctcttccagaacaacagggttggaagagaaggtgagggaaa
tattcttcctttgctatttctgtagaaaaggacaaactctcttccttcacatacataggtcaattgctagatcctagtgaagcctgagctta
acctactgttggaggcttaaagttcgacattaattgctacttttcttggtcagagttttaaataattaggttggtacaaaaaactgtgatta
cttttccaccaacctaataacatgctacaatttctgtaattattattttacactgtcaagacatagcaggtggtccgtttttgttattgtcaa
gaactgtcagactaaaaatgaactttacacttctttttaaatgatacattttctagaaaattcaatgaggtttaagagcaattgaaaagt
ctgatttcaagagagtctcatccaaaatgtactatatatttttccccaaagtccttggagttaattttgacaacaatttaaagtacactta
agtcttttgaagttaatgggtctgccacccaggttggagtgcagtggcgtgatctcagctcactgcaacctccgcctcccgggttca
agcgattctcctgcctcaacctcccaagtagctgggactacaggtgtgtgccaccacgcctggctaatttttgtatttttagtagagac
ggggtttctccatgttggccaggctggtctcgaactcctgacctcaggtgatccgcctgtctcagcctcccaaagtgctgggattaca
ggcatgagccaccgcgcccggcctgaagttaatttttatacccacctaatgttcattatggatcttgaaggtaaattaattctgcacta
aaattttacaatgctttacaaaatgactgtaggtggcccatatggaattcggtcaactgggccaatgacacatatgggattgcagtt
gaaattatccaattcctacttgatatttgtaagctgctgtgatagccagtataattgtactgtaagaatgtggtaaatagccggggccc
ggtggctcacgcctataatcccagcactttgggaagccgacgtgggcggatcacttgaggtcagtaggtagagaccagcccggt
caacacggcaaaacctcgtctctactaaaaatacaaaaattagccaggtgtggtggtacgcacctgtagtcccagctactcagg
aggctgaggcaggagaatcgcttgagcccatgaggtggatgttgcagtgagcaaagatcgcaccattgtactccagcctgggc
aacggagtaagactctgtttcaaaacaacaacaacaacaacaacaacagattggtaaatagagtaataataaaatcaaattaa
acttgcaaaaaatggccactttgctcccactggtggccaatggaggtcaaggacctggctgacctcctgcctaaaggcagaggtt
gttagccttcgcaatggactcaaatcagagggggagctttcaaaactcctgctgcccagactgaaccccagatcaatgaaacca
aaatctctggatacagggcttggcatttgtagcttttagagttcctaagtatctctactgtgcagccaaagttaagaatcagtgccttag
aacatcaacagttttttggtccttttgttaaaaagcacagtccgtttttttaggtggctagaaatgctccaggaagagctgaaatgtattt
accagccaccttggtttgattttagaaagcaaaatagaagttctaagtatgctttctctgaaaagctgagactgcagataagagtga
gggcagttgatggagttcattctcctctttcaatcactgcttctcatcctttcattataataatctaagaatctcagagattatgaaagag
aaagcagtcttatggaagaccccagactcacagaatattagggtgtgtttcacagggaaggatgtcattacccacagttagtctttg
aaacgcagttggacattatttgtaagtgcatcatagtgtcgcctccaggttccattgaggggaacgtcattccaatgcaacatctctg
agttcatctgggttattaaatggggttgagggatttgttatttttaaattagtagccccaatttaggactactcaagaccataggacaag
cctgtccaaccctcggcctgcgggctgcatatggccgaggacagctttgaatgcagcccaagacaaattcataaactttctgaaa
atattatgcatttgttttttagcccatcagctactgttagtgttagtgtattttatgtgtggcccaagacaattcttcttcttccagtgtggccc
agagaagctgaaagattggacacccctgctataagacacagtaatataaatacataacctgtggttctggattggcattagcaga
tacaggctgtgttgattttgcagaaagttacaaagagctgctagttggtgtgtatgtctaaaatcagtagatttcctgtggttctaagga
atgacaaagaatctggaagttctctgtggtagcctgctcagtgcagaaagggaacgtggaaaatccgccaccagcatttgagtct
tggaggttccacatagggctatcaggtctctgctgatcactgaaaccagatcatggccaactagccccttggcttcagccctccca
attcattaactactcaggtaaatctagggtcactttcaactctaccacctaccatctgagtgaccttgaaaacattcatctctctgagcc
tcaggtcccatgtctgtaaagcaggggcctcatggacttctttgggtttttttgtttttgtttttgtttctgaggattaaacaaatgctccctac
cctatttcccagcatccagtaacacagtttttcatatttttgtgtatgttaagtcaggacccatctctttaatgataagtgcacttaatgtgg
tcatgttttcttttgtcttccaaagctgttagtgaatccattgaatttgggatgggtaaaataaagtatctattattaattgtaaatttcatcta
aagtgacaaatcctacctgcataaccatttcttaatttcctttcatcatgtatcagtggtcaacattgttaactgcgaatgaatcagaat
ccatcaaaaattagaactatttccagtctggcaaaaattcagctctggttgaatccaaacattgtgctgaagcagctaagtaattca
actgaggagattaattacatgttataatcaataggttctcttgacacttcagtgttagggaacatcagcaagacccatcccaggaga
ccttgaaggaagcctttgaaagggagaatgaaggagtcatctttgcaaaatagctcctgcagcctgggaaaggagactaaaaa
ggtaaaaagctgttaattccaggaagacagctttacgcccctcccagaccacctgcactgcacactacgtggaatttattttagtct
cacatggcagcgtccctacctttgtgcccacacatctggtctccgccctggctgcagccctccccttcaggcgaattctgggtgtgtc
ctatctgctcattgcaactcccagcgaatgagttttcagcgaaggcagactttctgacctgttcttcaaactgcactggtcttttaaaaa
cgtgtttggtggccatcagcatccaatttcagaagaaagatttgggtgaggactgagagaggctgttgttgttgtgctgtctgtttcctt
cagaatctgcagaagaaaattggcaggtcatgtactgtggacctaaccaaaggacaaatgatgtatggaaaatagaaaaactg
ttgtgaaattgcttcctcattagcaataactgtatttggcagggagaggagaagttgggcacatttttttttcttttttttttcatgattcatac
gttttctttaaagaagtgggttttgcttttcactgggtgctctaagacaaccccagtgaaagatctggaccacgaagacccagtcatc
ctcataagggtgttcattgcagcaagctcaagggcatgccaggcaaaggccttttttctggcagcttgaacttgtctcagcagagg
gtttcacagaacaactgtcatttacctgttctctgctcttacttgattcgtttcccaggactgctgaaacaaagtaccacaaacttggtg
gatcaaaacagcagaaatatatcctctcacagttctggaaaccacaagtcagaaaccaatgtgttgttggcagggttggttccttct
taaggggctagagggaaaatctgtttcatgctcctctcccagcttctggtggtagctagcaattcttgatgctctctggcttgccgctgc
atctctctagccttcacctctcctcatgtgggtggccttctttcctgtgtgtctatttccaaattccccttttcttataaggggaccagttattg
gatcagggcccaccttaattcagtagatcccattttaacttgatgacatcagcaaagtccaaataaggttgtattcacaggtaccag
gggttagaacttcaagttatctattaggggacacaattcaacctaaaaactccccttttttgattctctattctgccacttctactcaatcc
aggttcttcacttcatcagctcccaatctaatacttatcttatttctagtaagcatctcttccttatcttaactggtccctggggcctggccc
gagccccattataccatcagctgttgacatcaagggtggacttctctttcggcacagaaggcacagggctgtaggcttcagccttct
ctgctttgctctgccccatctactgttcatccacctgctttccattttgctaaactttgtagaaaattcttgtcagctgttgtctcctcctacac
tttctttgatcttagaggattctattcttttactatggctttaatcggagcacccgactgttaggttcaaccaacagaagttggttgtgctctctc
actctttctttctctctctctctctttctctctatttgcatagtggtattttttttttcctctattttattggcagaattgccatttctctaagttattgt
agagttgctgtttctctattttatttgcatatttctcttctgccaggctggattgtttctattgattggttctgctgtaatgagggtgacttctcatt
agtatccttctcacttcatctgggaccagatgccctttgatatccttttggagccacaacttttggtagtcagaggcatgggtgtggctc
aaaggaagaacttggctcagaaggtgcagctcttgctgggcctttggtctctgctctgtcttctgagatcagtggctgctgggacctg
gggttcccccatgccgggcatggtcacacagcactcctatggacttgagcagagcaccctgcaaagtgagcattagcaatccatt
ccaactctgtgcagtcctgcacggaatatagaaggtggagcaatgacagtctccccaacttctctgcaagcaacctgctcaccatt
tcttgcccttcccatttatgtacttttcaaaatcaggttatttggaatttgtcgactcatgtttcttacttcagtacttttttgggagggcagcat
tagaaacctcaaactcttaactaaaaaatgtctttgggaatgttctggccattttcatggcccacaatttgctttaagctgctttagactc
tcccagaggctattttcatcccgaaagaacagagcagagctcaaaagactccagttttggtctctagcagcccctagaggatttcc
ccctcaattcctctctgccttgtatgaaatagaattggatttgaaatcggatgttgaggccttacctccaggctagtgaggccacaca
agatggatcctctggacccgcccaagtgtccacctaaacatgagttaccaactaacaatgttttgtttagcatgcaaagggagtgg
tctggaatctggccttgccctgacatattctccttgggcctttttaaaaaaataatttgtgttaatctgtagttaaaaattataataaggac
ctgacaaacactacctcagtcagatgatcaaggtacacataaatagtgaaagtcatgttgatagcatgcacccttcatatgatatg
gctagaatggccctgcacttctgtgatcttcctcccctagactcatcagctcgatctaatcataacaaaagcatcagataagtcccc
gcccagggacattctacataaccatttcccttcccagttatatttttctccacaatactttccaccatctaacattctatctttcaaaatgg
gcaagtattttagcctggtttgttcattgttttatctgcaactcaaatacagttcctgaaataaaatatctgcctaataaatatttaatgaat
gaatgaatatagcattgccttatccgtttaattgccacatggtatttcattgtgtgaacataatatcgtttatttacccagactactactcat
aggcatttagattatttccggtcttttgctattgctaacagcctttgcaatgaacatccttgtatacagacatttgcatatatgagggtgtgt
ctttaggatctacttctagaattgaaattgccaactccaagtatatgtttccaattgtgatagatattacacattaccctccatcttagag
gtggtgttaatttagattcctgccagcaaaatttaagagtgtttgtttccccatatcctcaactgcctaacagaatcagtgaaaaatggt
atgacagtgtaatttttgagtgaggttgagtatcttttcctatgctttaagagcaatttatgtttcctttttatgtgaactgtctgttaatatatttt
ttcaatttttctattgggttatttgtcttttcattaatgcatatacctgttacatatttataccaagtatgtattaaatactaacatattgatgaaa
cagagcaaaaagcctagaaatagatccaaataacagaagagttagtatgtgatacaggaagcctataaaatcagtgagcaaa
agaccatccaattaataacgttagggtaaatgggtctccatttagaaaaaaataatgtgggtctacacctcacattttatacctaaac
aattccagtgggataagaaaatgaaatcataaaaaattactaggaaaaagatgagaaaattgttcataaaactgaagtgtggaa
gatcctttatgccttacactgccctgagtgatctcattcatacccatggcttcaattgtcatgaatcccaaattcattcctctgtcagaact
ctcttctgagcttcagacccacatactcagctgcctactggacacctctacttgaatatcacaaactcaactcaaaagcaaacctgt
caaatttaattactagtagccctaccccaaacaatcttcctgctcagtgaatgacacccatccctccaggtgcacagaccaggaa
cctagaagtcactctgattgcatccctctccctcacaacctctacctccctttattcatccattgctatgtctctcaaatgtacctcccaa
atatctcttgaacgcgttcttttctatctctattgccaccaccctagttcaaactcccatcatctcatgactgaagttctgtgccctcttgcc
agtgaacactgtagaatcaatctaaacatggtgccaccctgcttaaaaaccttcaaaggctcacatcacttctcagatgaagagat
tggggagacgttggtaataggacacaaaatttcagttaggcaggaggaaaaagttctattgaagaactctattgtacaatatggtg
actatagttaataacaacatattatacacttgaaaatcactaagagagtccattttaagtgttctcatgaccaaaaaatgataagtat
atgaggtaatgcatatgtgaattagcttgactgaggcattctacatgtatacatatttcgaaacatcatgttgtacatcataaatgcata
cactttttagttgtcaatttaattaatattttttaaacctactctggcctttttttccttttttgagacgggtggtctctgtcccccatgctagagt
gcagtgcgcaatcatggctcactgcagcctccacctcccagtctcaggcgattctccagtctcagcctcccaagtagctgggacc
acaagcatgagccaccatgccccgctatttgtttttgtattttttgtagagatgggatctcgccacatggcccagtctggtgtccaactc
ctgagctccagtgatccacctgcctcagcttcccaaactgctgggattacaggcgtgagccactgtgcctggtccactctggtcttta
ctcaagtccctggctttctctcagtctcttaaacttatgtgcttagtaagatgaggactgaaaaatgtccacagaacatagtgacatg
gagatactgagaacctcaacgacatctccattagccacttcctctgtgccattccagtcctctgggccccactgtggcaagcagtcc
taccatggcaaacatgaaagctgatgtgccttgtcttagacccacaccatatctctctgaattcctgtcccagggcttctctggaggt
acagcctgggaaactcacgggaatagacacagggcctttgcacatgctgctcccttttcctgaaaaattcctttgacatcttggttgt
gccttacacatgcctactcaaccttaggattgcagttcaggtttcactccttttttttttttctttttgagacggagtttcactcttgttgcccag
gctggagtgcaatggtgtgatcctggctcaccacaacctctgcctcctgggttcaagtgattctcctgcctcaacctcctgagtagct
gggattatagtcatgcaccaccacgcccagctaattttgtatttttagtagagacagtgtttctctatgttggccaggctggtctcgaac
tcccgacctcaggtgatcggcccgcctcggcctaggttccacttctttatggaaatcttccccagttgccttgactaggccaaagtcc
cctcttcttaggctcttacagtgtcatgcacttcttttttatcacagtgtaaaccttgtaatgttgtgtttaagtcatatctgttgtacccatga
gactgggagccaattcatatattgtgagtgtaatcgaacagacttcccaggccacccactagctaatcaaggcagggatgagtcc
ggaaagtgactttgaaatctagcaatgttggaacttggaaatcacacaggctgagatctgctcaggtgcctgaacaaatatagca
ttgcctgtggcgtctccctcaaagtgccttgcatgtctgagccccgttgccccttcctttggtgtgcctgtgtctcccggtacagatgtga
agcctggagacctgtggctgcctctgcaggagctccatgttttcaagccataaatcatcttagaattcatagcatctagatatattagt
tttctattactgcagaacaaatcgctcccaaatgtagaggcttcaaagaatgcccattgattggccttaatttctgtaagttagaatctg
ggcaggtttgcctgagttctccactccaagtctcataaagccaagctgggctgtcatctggaggctctgagtaaaaatttgtttccag
gttcatccagattgtcaggtgatttcagttccttgcagttgttgttcgactcactaccccaccaccaccccgaaaacctcatttccttgct
agctgcctgcagagagccactctcagcttccacaggctgcttgcattccttgttgtggggccgctacctcctcaagccagaaatag
ggcatccagttcttctcatgcatcctacccctctgacttttccttctgccgataaccagaaaaaacgttccgccttcaaacgctcgtatg
attagactaagcccatccagataaattcccatatgccatatactataatgtcatcacagcagtaatacccgggacaaaattcatgg
gggtcatcttaaaattctgcctatcacaccaggtatagtagaggcttgttttagtgcaagttaaacattaagcagcaacatcacgata
gtgctgcatttgaaaataactactagcaactgaacatgtctgggagttctgctccactttaatttccatctcaaaaggagctgggttttc
cttggctgttacaaatgggcaataatgattgagcttaagaataatcaatgtccacataaaaatcttttataacatagtgagagtgtga
catataaaggtgttagttcaccggccctaaattttaggagaatttttaaaaaggcacttatctggtttaatccataataaagacatgag
ttgggctttagtgaaaaatctaggctggtttctgtgttcagtgaaagaagatttgagagttctcttaattacaacccttgatcaaaccta
ccacattaatctgtttattgcattgtatggttaccaaaagtgatatattcagccctctatttattaagaaacagttacagaaagtgaggc
actctcctgtgttactgagggtgcataaaaatataaagcaccatgtgtcttccctagagaagtttcaaaactagcaagcaaatagct
attaatgctaatgtttgtgtgatagggaacatatgagtagtaattattccacaaacaattttttgagtgctgtttacatttgaggcacagtt
caggcacgaggatttcaaaaggagattgtgtagcatgatggcttgttaaaaatatgattttggaatcagatttgctcaagtcccagtg
ctacagcataccatccttcaaaaaggtacttaagtctctgagtttgttttctcatctgcaaaatataaataataagaggacctactgcg
tcatgttcttgtgagcattaatgtgggtgatgaaatgtttatgaagcacttagcacaatacctgacattttgtttgttattattatcaacata
aagtgcccactttccagtcatgcaagaagaaaacataatatatgtcaccatagaagtatagaacaattgtgggaaataccagta
agagagatatagctgtataaataaggtaaagatgactgcctagaagatctaggatgataccatattagaagttgcatctgaactct
ccttggggactggccaaagtttcatcaagtgtcatgtcagtaggttggtgctataaatatatagcttgcaaagctatagacttactata
aaccatagctgtggtccagcttagactcattatggtggtggagtatcttgattaatggcctctgcagaagcttcccaggtcttctcatca
tcataatctcagatagcttcatcttcaacttccttttttttgttgtttttgagacagggtctcactctgtcatccaggatggagtgcagtggc
acaatcatggctcactgcagcctcgacctcaggagctcaagccatcctcccacttcagcctcccgagtagttgggactacaggca
tgcaccactacgcccggctaattttttcatttttttgtagagtcagggtctccctatgctgcccagtctggtctcaaactcctgggctcaa
accatctttccacctcggcctcccaaaatgttgggattacaggtgtgagccaccacacacagcccatcttcaacttcttttagcacca
tgaagctgaacatagtaaaaaagtaaaatcattctggacctaatctgatgcaatttatttaattgttaagtgaatgcacacatcaaaa
ttcatacaagtatggggcagcgctgctaatttatttacaaaacacctggcaaatactgctactctaatactgtgcttccacttttgattttc
cttagGAAAACATGTTCCTTCAGTCGTCAATGCTGACCTGCATTTTCCTGCTAATATCTGGT
TCCTGTGAGTTATGCGCCGAAGAAAATTTTTCTAGAAGCTATCCTTGTGATGAGAAAAAG
CAAAATGACTCAGTTATTGCAGAGTGCAGCAATCGTCGACTACAGGAAGTTCCCCAAAC
GGTGGGCAAATATGTGACAGAACTAGACCTGTCTGATAATTTCATCACACACATAACGA
ATGAATCATTTCAAGGGCTGCAAAATCTCACTAAAATAAATCTAAACCACAACCCCAATG
TACAGCACCAGAACGGAAATCCCGGTATACAATCAAATGGCTTGAATATCACAGACGGG
GCATTCCTCAACCTAAAAAACCTAAGGGAGTTACTGCTTGAAGACAACCAGTTACCCCA
AATACCCTCTGGTTTGCCAGAGTCTTTGACAGAACTTAGTCTAATTCAAAACAATATATA
CAACATAACTAAAGAGGGCATTTCAAGACTTATAAACTTGAAAAATCTCTATTTGGCCTG
GAACTGCTATTTTAACAAAGTTTGCGAGAAAACTAACATAGAAGATGGAGTATTTGAAAC
GCTGACAAATTTGGAGTTGCTATCACTATCTTTCAATTCTCTTTCACACGTGCCACCCAA
ACTGCCAAGCTCCCTACGCAAACTTTTTCTGAGCAACACCCAGATCAAATACATTAGTG
AAGAAGATTTCAAGGGATTGATAAATTTAACATTACTAGATTTAAGCGGGAACTGTCCGA
GGTGCTTCAATGCCCCATTTCCATGCGTGCCTTGTGATGGTGGTGCTTCAATTAATATA
GATCGTTTTGCTTTTCAAAACTTGACCCAACTTCGATACCTAAACCTCTCTAGCACTTCC
CTCAGGAAGATTAATGCTGCCTGGTTTAAAAATATGCCTCATCTGAAGGTGCTGGATCT
TGAATTCAACTATTTAGTGGGAGAAATAGCCTCTGGGGCATTTTTAACGATGCTGCCCC
GCTTAGAAATACTTGACTTGTCTTTTAACTATATAAAGGGGAGTTATCCACAGCATATTA
ATATTTCCAGAAACTTCTCTAAACTTTTGTCTCTACGGGCATTGCATTTAAGAGGTTATGT
GTTCCAGGAACTCAGAGAAGATGATTTCCAGCCCCTGATGCAGCTTCCAAACTTATCGA
CTATCAACTTGGGTATTAATTTTATTAAGCAAATCGATTTCAAACTTTTCCAAAATTTCTC
CAATCTGGAAATTATTTACTTGTCAGAAAACAGAATATCACCGTTGGTAAAAGATACCCG
GCAGAGTTATGCAAATAGTTCCTCTTTTCAACGTCATATCCGGAAACGACGCTCAACAG
ATTTTGAGTTTGACCCACATTCGAACTTTTATCATTTCACCCGTCCTTTAATAAAGCCACA
ATGTGCTGCTTATGGAAAAGCCTTAGATTTAAGCCTCAACAGTATTTTCTTCATTGGGCC
AAACCAATTTGAAAATCTTCCTGACATTGCCTGTTTAAATCTGTCTGCAAATAGCAATGC
TCAAGTGTTAAGTGGAACTGAATTTTCAGCCATTCCTCATGTCAAATATTTGGATTTGAC
AAACAATAGACTAGACTTTGATAATGCTAGTGCTCTTACTGAATTGTCCGACTTGGAAGT
TCTAGATCTCAGCTATAATTCACACTATTTCAGAATAGCAGGCGTAACACATCATCTAGA
ATTTATTCAAAATTTCACAAATCTAAAAGTTTTAAACTTGAGCCACAACAACATTTATACTT
TAACAGATAAGTATAACCTGGAAAGCAAGTCCCTGGTAGAATTAGTTTTCAGTGGCAAT
CGCCTTGACATTTTGTGGAATGATGATGACAACAGGTATATCTCCATTTTCAAAGGTCTC
AAGAATCTGACACGTCTGGATTTATCCCTTAATAGGCTGAAGCACATCCCAAATGAAGC
ATTCCTTAATTTGCCAGCGAGTCTCACTGAACTACATATAAATGATAATATGTTAAAGTTT
TTTAACTGGACATTACTCCAGCAGTTTCCTCGTCTCGAGTTGCTTGACTTACGTGGAAAC
AAACTACTCTTTTTAACTGATAGCCTATCTGACTTTACATCTTCCCTTCGGACACTGCTG
CTGAGTCATAACAGGATTTCCCACCTACCCTCTGGCTTTCTTTCTGAAGTCAGTAGTCTG
AAGCACCTCGATTTAAGTTCCAATCTGCTAAAAACAATCAACAAATCCGCACTTGAAACT
AAGACCACCACCAAATTATCTATGTTGGAACTACACGGAAACCCCTTTGAATGCACCTG
TGACATTGGAGATTTCCGAAGATGGATGGATGAACATCTGAATGTCAAAATTCCCAGAC
TGGTAGATGTCATTTGTGCCAGTCCTGGGGATCAAAGAGGGAAGAGTATTGTGAGTCT
GGAGCTAACAACTTGTGTTTCAGATGTCACTGCAGTGATATTATTTTTCTTCACGTTCTTT
ATCACCACCATGGTTATGTTGGCTGCCCTGGCTCACCATTTGTTTTACTGGGATGTTTG
GTTTATATATAATGTGTGTTTAGCTAAGGTAAAAGGCTACAGGTCTCTTTCCACATCCCA
AACTTTCTATGATGCTTACATTTCTTATGACACCAAAGATGCCTCTGTTACTGACTGGGT
GATAAATGAGCTGCGCTACCACCTTGAAGAGAGCCGAGACAAAAACGTTCTCCTTTGTC
TAGAGGAGAGGGATTGGGATCCGGGATTGGCCATCATCGACAACCTCATGCAGAGCAT
CAACCAAAGCAAGAAAACAGTATTTGTTTTAACCAAAAAATATGCAAAAAGCTGGAACTT
TAAAACAGCTTTTTACTTGGCTTTGCAGAGGCTAATGGATGAGAACATGGATGTGATTAT
ATTTATCCTGCTGGAGCCAGTGTTACAGCATTCTCAGTATTTGAGGCTACGGCAGCGGA
TCTGTAAGAGCTCCATCCTCCAGTGGCCTGACAACCCGAAGGCAGAAGGCTTGTTTTG
GCAAACTCTGAGAAATGTGGTCTTGACTGAAAATGATTCACGGTATAACAATATGTATGT
CGATTCCATTAAGCAATACTAACTGACGTTAAGTCATGATTTCGCGCCATAATAAAGATG
CAAAGGAATGACATTTCTGTATTAGTTATCTATTGCTATGTAACAAATTATCCCAAAACTT
AGTGGTTTAAAACAACACATTTGCTGGCCCACAGTTTTTGAGGGTCAGGAGTCCAGGCC
CAGCATAACTGGGTCCTCTGCTCAGGGTGTCTCAGAGGCTGCAATGTAGGTGTTCACC
AGAGACATAGGCATCACTGGGGTCACACTCATGTGGTTGTTTTCTGGATTCAATTCCTC
CTGGGCTATTGGCCAAAGGCTATACTCATGTAAGCCATGCGAGCCTCTCCCACAAGGC
AGCTTGCTTCATCAGAGCTAGCAAAAAAGAGAGGTTGCTAGCAAGATGAAGTCACAATC
TTTTGTAATCGAATCAAAAAAGTGATATCTCATCACTTTGGCCATATTCTATTTGTTAGAA
GTAAACCACAGGTCCCACCAGCTCCATGGGAGTGACCACCTCAGTCCAGGGAAAACAG
CTGAAGACCAAGATGGTGAGCTCTGATTGCTTCAGTTGGTCATCAACTATTTTCCCTTGA
CTGCTGTCCTGGGATGGCCTGCTATCTTGATGATAGATTGTGAATATCAGGAGGCAGG
GATCACTGTGGACCATCTTAGCAGTTGACCTAACACATCTTCTTTTCAATATCTAAGAAC
TTTTGCCACTGTGACTAATGGTCCTAATATTAAGCTGTTGTTTATATTTATCATATATCTA
TGGCTACATGGTTATATTATGCTGTGGTTGCGTTCGGTTTTATTTACAGTTGCTTTTACA
AATATTTGCTGTAACATTTGACTTCTAAGGTTTAGATGCCATTTAAGAACTGAGATGGAT
AGCTTTTAAAGCATCTTTTACTTCTTACCATTTTTTAAAAGTATGCAGCTAAATTCGAAGC
TTTTGGTCTATATTGTTAATTGCCATTGCTGTAAATCTTAAAATGAATGAATAAAAATGTT
TCATTTTACaagaggagtgtatgataaatatatcatagagaaattggtctttaatataaaagaaattgccatatacactgaattt
tttcagaactctttttaaaaaactatttggtagaaatcaaaggggaagcagttttcatgacacttttactttaagatacttattaatagata
aattctatcttgattccctactcagaagacataaagtcagaatgcctggctgttggtagcctttgtgcaattcccccaaatgaaacaa
ctttggcaaccctttccacttctactgtccccttggttcctctgcatcagtccatagcatcctctatccagtatgaatcttgagatatctaat
gaaatttacctgagaataactagaaattatccaagcataagaaaaggaagttgcttcagaatgaaaagaagataaacctccaat
ataccatctttcctttttagttaaatcttacagcatgagttaccttttaatatgtgcttctaagaaactgaccaaaataatgtgtcatagtgtt
atttaatacgcacaaagtggaaagcagtgcaagtttgccaaggacaatttaattttgtcacattgcatgctgttttgtgaccatgaag
agtttatacaaagatgtttatgcttgtgcttgttgaggtatagggacaaatatctaaaagcaagatcagatgggtgtggtatctcacac
ctataatccttggattaaaatctacctcaattgtaggactaccagttgaaccacatgcttcccactgccctcagcaaagggcacctta
gttagaggaaaggtagagcctttctatggaggaggaatttgtgaggtttgagttttatcagctacctgggagtcagaccctgatagat
tctccttcacactccctggaccttttcctgccaagtggaggctctcactcagaggaaatctccattcttttgatgcaggtcattcatactc
agatattctgcactgttcaagcaataaaaattgaatgagcacctattatgtacaccagttggcactgtgtcaaaatgtacttgtgcag
agaccttggatcattggtgacaggtcttcttctcctctgcatttttctcaagaccaggcctcagtgtagcatgtttccatggagtgaaag
aggggaaggaagagtgggctttggaaagtggcagctgtgtcatagcagtcagcctctgtgtatgtgaaggactttccagagcccc
cccactaaagcctccatgctcctcctgggactgccacagttcttgaaactatccatacagtcttcatgagttatttttaatttttttttcttcttt
tctctttcctccttttccccttttccccactccctagttagatctttaaaaatgcaattgtaacctttatcttcccttcaccagacactccctac
agggcaagcttatgtatacgcttacctaaaagctccagagccagaaatctctcccactcggggactgcctcaagagacagcagt
caatttacaacctaaagcatgcccacaacaaaactctctcccacctggaggatatcttgaggcaatggtcactttacaacctagttc
tgcctgcaatggcaccagctcaaccacctggtacataagacacaaaagcaagttgcatagacctcaccttctcactcccttccctg
catgccattaatgccaactccccctttaaaagcccctgctttctgccccaaaagcaaagtgatacccttaaagtcaggagcctata
cttcttccccctaagctaatttttggaataaaagtcattttattgagaacctccataaactgttggtgggaatataaattagtaaaccatg
atggagaacagtttggagtttcctcaaagaactaaaaatcgaattaccatatgacccagcaatcccactgctgggtatacaccca
aaagaaaggaagtaattatattgaagagatatctgcactcccatgtttgctgcagc
TRL10 genomic sequence

SEQ ID NO: 8

tcagcccatcatctacattaggtatttctcctaatgctattcctcccctagccccccaccccctgacaggccccggtgtgtgtcaatgt
gttctcattgttcaactcccagttatgagtgagaacatgtggtgtttggttttctgttcttgtattagtttgctgagaatgatggtttccagatt
catccatgtccctgcaaaggacatgaacacattcttttttatggctgcatagtattccatggtgtatatgtgccatattttctttatccagtct
atcattgatgggcatttgggttggttccaagtctttgctgttgtgaatagtgctgcaataaacacgtgtgtgcatgtatctttatagtagaa
tgatttataatcctttggatagatacccagtaatggcattgcaggatcaaatagtatttctagttctagatccttgaggaatcgccacatt
gtcttccacaattgttgagctaatttacacccccaccaacagtgtaaaagcgttcctatttctctacaccctctccagcacctgttgtctc
ctgactttttaatgatcaccattctaacaggcatgagatggtatctcattgtggttttgatttgcatttctctaatgaccagtgatcatgagc
tttttttcatatgtttgttggctgcataaatgtcttcttttgagaagtgcctgttcatatccttcacccactttttgatggggttgtttttttcttgtaa
attttttaatgttctttatagattctgggtattagccctttgtcagatggacagattgcaaaaattttctcccattctataggttgcctcaaac
agaggaatctttaaaatgtatgtcagaacctgtcattcctggactctaaatcttctgctggtttcttatttcagtcagaggaaaattgcca
agttcttataagatccgacctcttctctgattccgtcccctaactccactccaggtcttcctcacattttccaagcacatcaggatctttaa
atttgtacttgctgttctctctctctccaaaatactctttccccagacaacaagtgtcttgcttctttggcccctttagatttctgcatgaagat
cactatcagggaggccatttttgatcattctataaaaaagaaaatcactccccagtctctctctgtttcccttatcttagttatttttccttca
agacaatatcactgcctgatattggtccccacccaaatctcatcttgaactgtagctcccataattcccacatgttgtgggagggacc
tggtgggaagtaattgaatcatgggggcgggtctttcccatgctgttctcatgatagtgaataagtctcatgagatctggtggttttata
aagaggaggttccctgcacatgctctcttgcctgctgccacataagacatgactttgctcctcattcgccttccaccatgattgtgagg
cctccccaggcatgtggaattgtgagtcataacatataaacgtattgttttgtttgcagtctctcttttcttaacttctttctagaatataagc
tatgtagaaacagaaatctcttctgttcactgctacttccccagtgcctagaaaagtttctggcagaataggtacttaataaatatcttg
aataatgaatatcgtaaaatcttagtactccaactacctctgttctacgtctaatccaaccacgtgaagcctggcacatctcccaaa
gtcctcagaattctatatccttcaatttcctcatctatcaaatgggagtagtagtacttccctcacagagtatggtgataaataaatgag
ataatatacatgaagcaattagtatgtatcttggcacattgaaatctaacctgaaagctttgattctatgccataacagaattcagca
gctgaatatcaagacctttgaattcaacaagaagttaagacatttatagttgtctaacaacagactgaagattGTGGCTTGG
TATTCACTGGCAGGTTTCAGACATTTAGATCTTTCTTTTAATGACTAACACCATGCCTATC
TGTGGAGAAGCTGGCAACATGTCACACCTGGAAATTGTTTTTCAACATTAATACTATTAT
TTGGCAGTAATCCAGATTGCTTTTGCCACCAACCTGAAGACATATAGAGGCAGAAGGAC
AGGAATAATTCTATTTGTTTCCTGTTTTGAAACTTCCATCTGTAAGgtaagtgttgaaagtcagatat
tggctccagggactttctatatccacaaatacaaaaattgaggggtaactccttgatatcaagtcaaaggctcacaatgtctggtaa
taaaacaaattactttcaattttcttgaaatcttcagGCTATCAAAAGGAGATGTGAGAGAGGGTATTGAGT
CTGGCCTGACAATGCAGTTCTTAAACCAAAGGTCCATTATGCTTCTCCTCTCTGAGAATC
CTGACTTACCTCAACAACGGAGACATGGCACAGTAGCCAGCTTGGAGACTTCTCAGCC
AATGCTCTGAGATCAAGTCGAAGACCCAATATACAGgttggaaccttactccaacctcttgatgaatgtagt
cagatgttggcattttttttgcaaataaaaatcctacaggatttaacaaaccaaataaaaatctaatattatatacttttttttagGGTT
TTGAGCTCATCTTCATCATTCATATGAGGAAATAAGTGGTAAAATCCTTGGAAATACAAT
GAGACTCATCAGAAACATTTACATATTTTGTAGTATTGTTATGACAGCAGAGGGTGATGC
TCCAGAGCTGCCAGAAGAAAGGGAACTGATGACCAACTGCTCCAACATGTCTCTAAGAA
AGGTTCCCGCAGACTTGACCCCAGCCACAACGACACTGGATTTATCCTATAACCTCCTT
TTTCAACTCCAGAGTTCAGATTTTCATTCTGTCTCCAAACTGAGAGTTTTGATTCTATGC
CATAACAGAATTCAACAGCTGGATCTCAAAACCTTTGAATTCAACAAGGAGTTAAGATAT
TTAGATTTGTCTAATAACAGACTGAAGAGTGTAACTTGGTATTTACTGGCAGGTCTCAGG
TATTTAGATCTTTCTTTTAATGACTTTGACACCATGCCTATCTGTGAGGAAGCTGGCAAC
ATGTCACACCTGGAAATCCTAGGTTTGAGTGGGGCAAAAATACAAAAATCAGATTTCCA
GAAAATTGCTCATCTGCATCTAAATACTGTCTTCTTAGGATTCAGAACTCTTCCTCATTAT
GAAGAAGGTAGCCTGCCCATCTTAAACACAACAAAACTGCACATTGTTTTACCAATGGA
CACAAATTTCTGGGTTCTTTTGCGTGATGGAATCAAGACTTCAAAAATATTAGAAATGAC
AAATATAGATGGCAAAAGCCAATTTGTAAGTTATGAAATGCAACGAAATCTTAGTTTAGA
AAATGCTAAGACATCGGTTCTATTGCTTAATAAAGTTGATTTACTCTGGGACGACCTTTT
CCTTATCTTACAATTTGTTTGGCATACATCAGTGGAACACTTTCAGATCCGAAATGTGAC
TTTTGGTGGTAAGGCTTATCTTGACCACAATTCATTTGACTACTCAAATACTGTAATGAG
AACTATAAAATTGGAGCATGTACATTTCAGAGTGTTTTACATTCAACAGGATAAAATCTAT
TTGCTTTTGACCAAAATGGACATAGAAAACCTGACAATATCAAATGCACAAATGCCACAC
ATGCTTTTCCCGAATTATCCTACGAAATTCCAATATTTAAATTTTGCCAATAATATCTTAA
CAGACGAGTTGTTTAAAAGAACTATCCAACTGCCTCACTTGAAAACTCTCATTTTGAATG
GCAATAAACTGGAGACACTTTCTTTAGTAAGTTGCTTTGCTAACAACACACCCTTGGAAC
ACTTGGATCTGAGTCAAAATCTATTACAACATAAAAATGATGAAAATTGCTCATGGCCAG
AAACTGTGGTCAATATGAATCTGTCATACAATAAATTGTCTGATTCTGTCTTCAGGTGCT
TGCCCAAAAGTATTCAAATACTTGACCTAAATAATAACCAAATCCAAACTGTACCTAAAG
AGACTATTCATCTGATGGCCTTACGAGAACTAAATATTGCATTTAATTTTCTAACTGATCT
CCCTGGATGCAGTCATTTCAGTAGACTTTCAGTTCTGAACATTGAAATGAACTTCATTCT
CAGCCCATCTCTGGATTTTGTTCAGAGCTGCCAGGAAGTTAAAACTCTAAATGCGGGAA
GAAATCCATTCCGGTGTACCTGTGAATTAAAAAATTTCATTCAGCTTGAAACATATTCAG
AGGTCATGATGGTTGGATGGTCAGATTCATACACCTGTGAATACCCTTTAAACCTAAGG
GGAACTAGGTTAAAAGACGTTCATCTCCACGAATTATCTTGCAACACAGCTCTGTTGATT
GTCACCATTGTGGTTATTATGCTAGTTCTGGGGTTGGCTGTGGCCTTCTGCTGTCTCCA
CTTTGATCTGCCCTGGTATCTCAGGATGCTAGGTCAATGCACACAAACATGGCACAGGG
TTAGGAAAACAACCCAAGAACAACTCAAGAGAAATGTCCGATTCCACGCATTTATTTCAT
ACAGTGAACATGATTCTCTGTGGGTGAAGAATGAATTGATCCCCAATCTAGAGAAGGAA
GATGGTTCTATCTTGATTTGCCTTTATGAAAGCTACTTTGACCCTGGCAAAAGCATTAGT
GAAAATATTGTAAGCTTCATTGAGAAAAGCTATAAGTCCATCTTTGTTTTGTCTCCCAACT
TTGTCCAGAATGAGTGGTGCCATTATGAATTCTACTTTGCCCACCACAATCTCTTCCATG
AAAATTCTGATCATATAATTCTTATCTTACTGGAACCCATTCCATTCTATTGCATTCCCAC
CAGGTATCATAAACTGAAAGCTCTCCTGGAAAAAAAAGCATACTTGGAATGGCCCAAGG
ATAGGCGTAAATGTGGGCTTTTCTGGGCAAACCTTCGAGCTGCTATTAATGTTAATGTAT
TAGCCACCAGAGAAATGTATGAACTGCAGACATTCACAGAGTTAAATGAAGAGTCTCGA
GGTTCTACAATCTCTCTGATGAGAACAGATTGTCTATAAAATCCCACAGTCCTTGGGAA
GTTGGGGACCACATACACTGTTGGGATGTACATTGATACAACCTTTATGATGGCAATTT
Gacaatatttattaaaataaaaaatggttattcccttcatatcagtttctagaaggatttctaagaatgtatcctatagaaacaccttca
caagtttataagggcttatggaaaaaggtgttcatcccaggattgtttataatcatgaaaaatgtggccaggtgcagtggctcactct
tgtaatcccagcactatgggaggccaaggtgggtgacccacgaggtcaagagatggagaccatcctggccaacatggtgaaa
ccctgtctctactaaaaatacaaaaattagctgggcgtgatggtgcacgcctgtagtcccagctacttgggaggctgaggcagga
gaatcgcttgaacccgggaggtggcagttgcagtgagctgagatcgagccactgcactccagcctggtgacagagcgagactc
catctcaaaaaaaagaaaaaaaaaaaagaaaaaaatggaaaacatcctcatggccacaaaataaggtctaattcaataaatt
atagtacattaatgtaatataatattacatgccactaaaaagaataaggtagctgtatatttcctggtatggaaaaaacatattaatat
gttataaactattaggttggtgcaaaactaattgtggtttttgccattgaaatggcattgaaataaaagtgtaaagaaatctataccag
atgtagtaacagtggtttgggtctgggaggttggattacagggagcatttgatttctatgttgtgtatttctataatgtttgaattgtttagaa
tgaatctgtatttcttttataagtagaaaaaaaataaagatagtttttacagcctacacatcctactcatttggcttgattcttctttctggtct
cacaggtcacaggaagaaaagcactcctgaaatataatttttgcaaaattatatttcaaaaatgacaattttgcaaaattatatttca
aaaacaaacatcatgtcacttctctggttagaaaaaaattttgtggcttaaacacatgattcagggagagaatgtcatgctcctttaa
gatctgacagcaatctccttttatatccttgcatcttctttatttttaatttttagagactagctcttgctctgtcacccaggctggaatgcagt
ggtgcgatcatagctcactgcagtattgagctcctggcctcaaatgatcctcctgtcttggactcccgaagtgctgggattacaggtg
tgagccaccacacccagcccctccttgcatcctatcattgggccctatggagctactggcccttccccagaactttcagtgttctttca
tggctccagagcccagatttcacatcatgcctgctgtaatgccttccctacttgggtttgttcaggaaatcttacagttctctcaggaca
caatccacatatcgactcttctttgaaatcatcctcactctttcccgtaagcatgatgcttcttgattctcttccacactttggacatatttct
atcaccaacctaattatgtacatttttaaagtttcaatttcccactagactatgaactcctcaaaggctaagacagacttacatctgcc
cttgtgtctgcagcagtccctggttcagaactggtgctcaaagaatgtttatggaatggatgttgggttggctagaggagcttagtgg
gaactcaactggcttaaggatagatggtggaatttaaaggcatattctgagaagctcaggaagagcaggaataggtaaaactca
ggtaagaagacagagaatccagaattgtaggattcctaagtagagctcacgtcatgtgaaattgccaaaatttggttgctctcgac
ctagaaaagcatctacttttaaaaatctcattccatctgtattagggttctctagagggacagaactaataggatatatatatatatata
tatatcacacaatactatatctatatctatatctatatctatatatatatattgc
SFRS8 genomic sequence

SEQ ID NO: 9

ATTTTGTGGCCCGCTATGGCGGCGGTGTTGAGGTTGGGTACGGGATGCGGGGTCTTTG
ACTGAAGGGGTAGGCCAAGTGGAGGTATCAGGGACGTCGCGCGGCACAGAAGAGGAC
CAGCCTGGACGCCGGGGACGCTGTCATGTACGGCGCGAGCGGGGGCCGCGCCAAAC
CCGAGAGGAAAAGCGGCGCGAAGGAGGAGGCCGGGCCAGGCGGTGCCGGCGGTGG
GGGCAGCCGAGTGGAGCTCTTGGTTTTCGGCTATGCCTGCAAGCTGTTCCGGGACGAC
GAGCGGGCCCTGGCTCAGGAACAGGGACAGCACCTCATCCCCTGGATGGGGGACCAC
AAGATCCTCATCGACAGgtcggttcctctccccacccgtcgatccttcccttccctcacccgcttgatctcgtctgatgttga
cttgactgcaaggactgcagagagttttctggagccagcggggatctgggggacaccccctcccctgtccccacctcctcctggt
gttctggtggggagggggacggtgaaacctgccctaaggcactggctggaattgcgtgccgcgtccgtctccggagggatcgtct
ctggtcccgcagcccctctcgacccctcaccctgtcgctgggctgcagttggcgattccgcgcggtgaaagcagccagtgccca
gggtcttttcctgagtgcacctgggcctgccgcccggcgatgccatggggtcgtgcgctgcttttctacttgccgcgctctcactgctc
ggtgtactgggagggtaccctgggaggcgtgcctttattcttccgaaccgccgctcactgagacagtggctagaagtgtctcttgga
cctgtgagttagccttaacctgttatgcccccagagccctcagtggagcgcccgtactttgccggcatgacgtttgatttcccggtgat
aatccgacgagtttgacagattgaggtagtgagcaaagttgcccgtcagttggtggccacttgacttcgtgcggaccctggccttgc
tcttggaagagatagtgttcttagggctggtttcactgtctcttaagactgaagggtggagctgggatatagatgtgttgtttcttttcaaa
tcaaacctgctttaggtcgtcactcgagggtgtctagcgattatgggcagtgggggcctgggattagggatttctaaaggcgtttgat
ttgaaaaggataacattacatgatgtaggtggtttgctcccctctttcttcccattttttcaatcccccttcccctcgtctctgggttttgggg
ttgttgtggggtggttttttttcttttttgcctgttcggctacttctggggcccggactgaaaagctaaccatgaccaaccattaaactgtg
gaatagtctctccacgtgaagaaagcccatcgtttgagaccattaaaactggattcttcatagccctggagcatgactgtagggat
gacctctgagctggccagaatggacacattaatgaccaaataggcctttttccatccctgacgtttccttttggaattagagctcgaa
aacgagaactggtgaagggagggccgcggaatcagatcatgtctggatctgatggctccgtgctgtgctcaagcgtgctgtgcct
tcacaccatggtttatattgaatgtgtcgcctgagttgtcaggcttgcttttccaaagtgtcacttgtgttatttatcattaaagtttggtaag
caatgaagtctgagctctttgtacagttttcctatcattctgtacatgatttgagttaggtcttccaaaactggtggggagcaaacgccg
cacatgtacatgtataatatttttaataataatctacatttgtaagttaaggaggtttacatcaaaatccaattaattttgaaatttaatga
aatgcagtaacttgactaccttggaattttgggcctttttcctgtaaatgtcttttttggtctacattaatatttttggttcccattacaaaagtc
agcattaaaaaaataagcagacttttgtttgtttctctacatttgtttttgaaaccctaaacctgagtgttttaagtaaagttcactaactc
attcatttattatctgacagttacacgttgacagcatcctcattgaatcctttatgttaaaagcatagcagaaagtgctcccattacttatt
tggccaaactactgtttggtccatgcaagagaaacatggaagtgtcttcatgtatgttatttccttgagggtataaagttcagaagga
aatattgataccatatcttctaatagttttgctctgttcccagtgaacctccttaaactgcatgtatatgtcactgttccaatgtatgtgtgtc
tctctatcacgtaacccagcacttatttcctcagccaagtggctagggggcgagcctagccaagattttacctccaatggacgcaa
gtttctttggtgaagatctctcctgagagttcgggactagcagaaagaagcgaggaaatttcgaccgtttggttcttacggataggta
tttatgtatttggtttgtgtgaatgtaagctatgatatttaacttttcagaaaaaaataataattttttgcaagtggcattgaatggttgacca
aaccataatggtaagaactgccagtgaagtgggtaccatttttgctattaatggattgtttgcctttagttataaatgttatcttactgtgg
aaaggaatttagagtttgttaagataacttgagtttaaaagtaggtgagatatgactatccaaattaaatataaatctgggaagagtt
tttatacttgttttaatatttttgtttattttaatcggtaagtatgtgtgtgtatatatatatacatatataaaacatcacacacgcacaccagt
ctagacgttaatttccttttattgaccagcttgttcacattacagATATGATGGACGTGGTCACCTGCATGACCTT
TCTGAGTACGATGCTGAGTATTCCACGTGGAACAGAGATTATCAGCTGTCTGAAGAGGA
GGCGCGAATAGAGGCCCTGTGTGATGAAGAGAGgtatttagccttgcatacggacttgcttgaggaggag
gcaaggcaaggtactgctcaagacaaacttacttcagcaacaaactttttaaaatttttaagtatttaaaaatttactcccattcatttttt
tatactcactctttctgatattatcttgacagtacccagtggattggaaaaacaggagtctttgcgttctgagaggacctcaggatagtt
tatatatagagccacaaagaattttcccagcttttgagggcagactgggatttgaaaaaaacaaaaaccaaactctttaactgttctt
ctttaacagtatcgtataaataaaattgatgttcttgtctttgccgtaacagtctttaatacagttcttaatcccaaaattttctcagcagga
agaaattttccacaaaagacgtgtattcagctgtctgtgggtaaacatgtactgacaaaagtacataatgatagatataaagtgtga
atttttaaaactattttacctcaaaagtaggttgaaaaaagtatgttgtatgctttactgatagctacaactttagaaatatataaagttttt
ctcagtaattttctatttttgttgataaaattctcatttttattcaagAGGAAGAATACAAGCGATTGAGTGAAGCACT
AGCAGAGGATGGGAGCTACAATGCCGTGGGGTTCACTTACGGTAGCGACTATTACGAC
CCGTCAGAGCCGACGGAGGAGGAGGAGCCTTCCAAACAGAGAGgtgagtggggagctgcctgg
actgctggtgtagggctacacgtgtacgcacaggctgcatgcaccgtggtccagtctgcagaacacatctctggcactcatgata
gcaccactatgaccacaggagaaaacgggagtgatattccttcttttggtaaaacgaagttaaaaactagaatgattaatggagg
tggaaagtgaatgcgttggattatttatttctcattgattcgggtaacagaattactcattcaggattatttgtttctagattggtaacatgtt
cattaatatcctcagggattcattcttgaggcagtgaaagaataggtgttaactgggataagttaacatcaccgccctctcactgac
ctgcttccccatatccctccacaactgagacagtgacacatgcccagtggaaggacacagtgagggagtttctactccccagaa
aacagcacagcttcctggtagccttgatgccacctagggcatatacttaccacagtattttaaattaaagatttggaatttatgcttttct
ggattaacatgggaaactttgaatataaaaaatagtgctgctgaaaaacctgggctcgtgtagtatagacacaaatatcctcaatc
acttcactaagcgtcgagagctccactaccacagcgctgcatcatggtcagtcgttaattagcagtaatgctaacatgaacctgac
accttaaagacgggtcagtatattcaggatattctgtttaaaaagaagaagaacattaacttagaaacattcaaatgtttacattaca
tcaaatggagatttaattgtagagctaatttaatctgttattctgaacttcatcggtttctccttaagtaacactttttatctttttaaatttttttat
taaaatacacaataatttaaaaaaagagatggggtctcactgtgttgcccaagctagtctcaaactcctgagctcaagtgatccta
ctgccttggcctcccaaagcactgggattacaggcatgagccaccacatccagccaagtgacacatcttttaacaagtagaagc
aattatagcactttagtagtaaagcaaaatgatgtttgcccttccatcctgtgactgcactatggttctacccatcggcactctccaag
ggctgcgatcctaacggaatgataggacgtggggcaaacgcacacaccggctttccttttgccctgtctttagtcctgctccttacttt
gtgggcacaagaattactgttgcacagctctattttatgagcttttagagaaactttcaagtgtaattgtaattatactgagttaaaggc
cagttaaggtatttaagactttttgcattgactttcaaacctacccatccctcagaagttacgatgcactagaaatgttctatcaggtct
aaaacgtaaacacccatttatttatccagaataagctctccttcctcgggttctggatagttctgattttgttgtcttatctctaagccaca
cacatgagttcagctttctatctgtggtgtttttatcagaaggaaggaatagatactatagccacttcacaaataaagagttgaaata
cagtcagcttattgggtccacatctgtggattcaaccaaccacagatctaaagtattggaaagaaaataacaaagttttattggaac
acagcaatgctcatttgtttacatattgtctgttgccactttcgcacttcagcaacagagttgaagaaatgaaacaccatatggccca
caaaaccagaaatatttattaatactgtctggcattttatagagtttgtcagcccctattctagatgatggaccattgtctcggcgtaatt
attgggctaaatgatgttcagtttgttataattattgaatcttgagaacttcagcatgacttagcttatcatctgagtattagtttgctttccc
cttaagataaagttctctttagtattttacaatgttacttcttttctttctgtaatcgtgttctcagaacattgccttatatactgattaatttcgtta
atggaaattgggcccacataaaacttagagcttgacatttcgtgtttaacttgcattaatataagtgaaacacctaacacacacaca
cacatacgtgcatattgtaatagaatccagtaccactaacagccccattgagcgtcacattctgttaaaataaaattttttttcctgagc
catcaatatgtctacgtatgtcttgattttcaaaattactgtattgtattgtttgttagtattttaaagccttgtgatactagccaaaagcatttt
gatggtgcctccatctctgatctttactattttcagtcaagtttttatcctttagatgttcataatttttcatcattattctatatccatttttttccctc
ttttttaggggaataatggggcggggacaggccctcactgctatatatccattttttaaacaaaaggttatttgaatttatttaaatctga
gtttgtagtgcaatggttggtttttattttgtgctactaaagctgtttttttgtaaataaaggtatatataagaatagaccaaatctgtttaac
ccatcaatcccaaaaagctatttcaattaaaatgccttgatttttatgaataacttaacattaaggagaagctatttgcctagacaatgt
tttaatcatttttttcattttaggaaaatatagtaaaagttgtatttttaaatttactttgttttacttttttgagacagagtctcgctctgtcaccc
aggctggaatgcagtggtgcggtgtcagctcactgcaacctccacctcccgggttcaagcaattcttgtgctttagcctcccaaata
gctgggattacaggcgcccgccaccacaactggctaatttttctatttttaatggagacaggatttcaccatgttggccagactggtct
caaactcctgacctcaagtgatccgcctgcctcggcctcccaaagtgctgggattccaggcgtgagccaccacacccggttaca
ggtgtgagccactgagcccggcttcatctctgatttttgaaagaacaggggactcaaacaaatggatgggacggtgttaaataact
gtaacttaatagggattgtaatcaacttatatctgatcagactggaatacccaagtttttgtataccaggaaacctgcttaaaattcttct
ttggtttcacggaatgaggtttgacaggagatctttgcaaattattgatcgcttcaagagcctttactgtatatgatagaaacacttatttt
gatgaagatttaaggtttgtttctttaatgtcatctgtttggaaataagaacctcaatagatcattgaaatccttaaaaatgttaccttttta
aagtttgctatgatatttttgtacatttcagtgtgtctttttaaactggtaatcatctgagttactgagatgtacttaggtaccttagaataca
gaaagataatgtgtagtacgttgtctaccacatagtagacaagtatttgttaagtgaatggttaatgaatacatagaaatggaaaaa
taattgattatttgtgaaagaggtagtttgcttgggtggaggaatcttgatagttatgcccaggtggtttacaattcaaagatgaaaatc
agttatctaggaattgactaccttatgtagtgtcatgctgtcaggaatccacagaaatagttggagagaaatcttagcgataccaatt
aaatacatacatacctgaaagagcagcggaggggaaaaggaacatggattgagcacctgctaagtgtggaggattatggtag
agatgttcacatgggctatctcacctaagccccacttggccctgggagtggatgtgatgacttcacaaccagtgaggaaacagag
aacagcagaatcccggagctaacaagcggcagggcaggagtttaactacagttaacccttgaacaacacaggtttgaattgcg
taggtccgcttgtatgaggatttttttcagccaaacactgatcagattgagggatgtgagacccgcatatatggagggtcagcttcttt
atatatgtgggttcaaatggaccaaatgcagaattcgagcacgtgtggatattggtgtctgcaagggtcctagaaccagtcccctg
cgtataccaagggaataccgtgcagtctgaccctatacacactccttctgctgcaccccacctccccccaccaccccacctcattt
ggaattttgtaaagtgagtttcacttgcgttgtgggtagaagagaaagctgaagatggcttctaggagtaacaacagaaggtaaa
gaagcagggacagccagtcacatgcttccatcttgctgcctgctgttagcaggtgccttcctccctgcattgttctgaattttttaattttc
tttttatgcagAAAAAAATGAGGCCGAAAATTTAGAGGAAAATGAAGAGCCCTTCGTTGCCCC
CTTAGGATTGAGCGTCCCGTCTGACGTGGAGTTGgtatgtgtcctgcatgagcactagttgtcgtcattatta
tttatcataattcactcctgcttgtgggaaagctcaataatgattatagctgctttttaggcatataatgctttaaaatggtttgtgagttaat
ggagaaaaagatcacaccctatttattttccccaaaggaaaagggaagaattatagcaaaagagctagactggagcatcagg
gacttgaggagttgggtgtgattcagcggccatgtagttgaatccccagattttactagcttagaaaaactaaatcaggatcagtgg
cgagtgggctgctccccacacatagatgtaaaagcactcaagatcaagacagcgttgatttcagtaacgttgctttgttctggcttttt
aaagtgtgatttttggggtcacttcacgttacattttcttagcagttttctgtgttgtgataggtccctgtggcatctccaggcccaggcca
cccttccactgatgaaggggaattccaccctggttttcctcatctgagggctttgcaactggttactgtctgttcagcattgacctttcctg
ctatttcagttatatcacattaaattaagttataacaggtgttaaagcccaaaccaggatttttcctttttttctgaatattcattgagggatt
tccccattccagtcactgtgctaagctgttttttacatattatcttatttaatgctaacaaccctataagcgaactactgtttactatccctct
tgtgccagtgaagaaactgagacttaggaaagtcaagaatttggctaataaatagcaactgggaacgttggtcttaactatgatgc
catcttcagtcaccgtgctttatgggatttttatatgtttactgggaaggttgaaaatctttttgttgtgtgtgtatacttgggaaggactctta
agtgttcgtgcctagcaggaagttttttcttggacattttcgtaactggattgcaagtggcatcgatgcaggcattctcaattcttgtttgtg
tcccacatcctgaatcactcactgcatggtagatgccgggaaagctccgcacagagagagcatctctcacctcccactgcgatc
actcgctgccactctaattgagttcagcgtgaatttgatggttcttacccttcattaatctgatgaagggcaatataaaaatagccctttt
aattcctgcctccaacgctttccttctcttcccttattcatttacatatcctctccctctcttttattctttcaaatatgggtaaaataactttttgg
attttgcctagtataattactacttgtattggtctgttctcacacagctataaaaaaatacctgagactgggtaatttataaagaagagg
tttaattggctcacgattctgcaggctgtacaggaagccgggcagcatctgcttctggggaggcctcaggaaacttatagtcatgg
cggaaggtgaggagggagcaggcatgtctaagcatgtccagagcaggaggaaggcagggaggtgctacacactttcaaatg
accagatcttacgacagctcattcactgtctatcacgggagcagcaccgaggagatggtgcgaaactattcatgaaggatccac
cccatgattcattcacctcccaccaggcactgcctccaacattgggaatgacaattctacaggagatttggctggggacacagatc
caaactgatccaaactatattactacatatgtttgtttctccatttctagtattgatcattttgctgtagttaaagctgaaattacccaaaga
tttgatatcctgagacttgtattaatatattttccatgtattatatatattgtattcctatttgttctgaaatatgtttattatgcatgagagacac
attaacatgaagctttaaaaaatcacagttgctccatttttattaaatgctaagtgctccatctctatttaatgctaaaaagtttatatgaa
gttgactatatggaattttacttgtttttagtgttaaaaattttttaattttttattcaaatttaaatatagaggtacaatggaattgtgttgcctta
attcctattaaaatatttaatggctttgtgttctcagccaaaataagcatcactaagctcttgatagtctgccagatcaaacatacttgtc
actcattggagagcaaagtaagtcttagtgtgtagcaacttgctgtcttatcattagagtttcttctaatgatattatagaaaggcctctt
gaatgttgttttgactttgtggaaactgagtgcttgattgagtctctcatttgcgtctttcatttattttatggcagtgtcagtatttcattctcat
aattattatgtgttttttggcagtaattcattgtgtaaattatacaccgtggtgtccatgttagtggagaaaatgtagaagacagaagtgt
ctgcattataagttgttttagtgactaggcctcagaattgttgaattgtggttaagtagactattgctgcttaagggggcaggacatggtt
tgactcactgacaagagaagattggagtgattgggaaagacagcaggtacttcaggaggttcttggtttttaaactaactgttggttt
agaacctaatgatgacaggatccttgaggcttttggatgaagagtaagaagtagttagaaattacagcaccccaggctgggtac
agtggctcacacctgtaatcccagctctttgggagactaaggttggaggatcacttgaggccaggagttcaagactagcctgggc
aacatagtgagatcctgtctctacaaacaagtaaaaataataggccagtgtggtggcacgtgcctgtagtcccagacctgttagg
gcgcagttccaagggaaatgtgcttgctcgaacacattttatggaaagtggggaaggattcgatagttgctgttgtgtgcaacgctt
attctgttgatgaataataacatagaaccagcctttatgaagcacttactgtgtaccagacagtgtactaagtgcttctctaggcatat
ctctcagttaatactcaaaataattttacaggccaggtgcagcggctcatgtctgtgatcccagcactttgggaggccgaggtggg
agaatcgcttgagcccaggagttcaagaccagcctgggcaacatggtgaaactccgcctctacaaaaaatgaaaaaaattag
ccaggcgtggtggtacatgcctgtagtctcagctactcaggaggccaaggcgggaggatggtttgagccctggaggtggaggtt
gtagtgagctaatacggtactgctgcactccagcctagacaacagagccagatcctgtctcaaaaaaataaaaatacaaaaat
aactctatgaagcaaatacagttgttgccagatgttaaagtttagaaagttaagagtaactgccctaagttacatgtgtgagggtca
ggcctggggttctagccaagggaccgactccagagctctgaaccactaaagttaagctttatcgaatttgtgcagattagagcatt
atttcatcataatttaggtactgtattgtcacagaaggtgggtggggagggaaaaaatgttgatttattcttgaattactgtggagtgac
tggccttttgttcagattcgtaaggactcttgacgtctaatgagccttaactcttggtccccaatgtgtcttgcaggtattttctccccgca
ctttgttttctaagtgattgtacgactctctgtgcagaatttaagtatagagtgatatatgtccctctattccttatggcttcagaattttaaa
gcttattttggaaggcttcccacccacaagagtttgaaaatattttcctatatttactgctggtacttctgtatttgtatttttgcctttgaatttt
gactcaatctgatacttacttagggctctgggcaaagcaggtatttgattttttttctcccctaaccccgtgaggagagggctggcccc
actgcccctggggttccttgctaatttcccctcttcatgtcatgccacctccttcctggtcccccgatgggtctgagctcaagccttttcc
aaagctcttaggaaaccgtgcatttgtgtgggttttctgacctgtagatacctttctcctgtttcccatgcctcctctggagcttaaattca
cattaatggaatttccagggaaggggaggaggtgttagcccccttccggggctctgtcggcttccttaaacaaaaggcttccccac
tggatttaaattagaaaaacagttttccttttcttaggccattgtaacatatgcatatttatatgttgactgtattttttaaatctcattgtgtagt
aggagtgatcatgatattcttaaatggaaaatgatttacctaaagtctgcctaataaagtaaagaatggctttttccaaaccagatac
tttttaatcttattcagggttggtagccaaccttgaaatatgtccgtaagatgctttgtttttttgtaaaattacttacacattgctttttaacca
tggtaatagaagtagtatataacaaataggcctacttttaagaaattttggatcaacaagttggtttggtcaataaagaaagcctaa
actgggccagtatcatattttcctttaagggtcattactatgaagtgatcattaattattgatgtgtttatggaatattctgttttaacaatga
ataaccagagcccctgaaaatccaagtcgtggacatgcatacagtgggcctttaacatggaatttaaattatttggggattaatga
ataattgtagacatctatcctttttaagtgtgaaggatgttattagttcaaaaattaaattagagaattaagccatttatattttatgaagg
atgaagccctgaattcttaaccatccatttttaatgagaataattgccagatttatttgcaaataatttcctagtgttaatctctttgttgata
tgaaaggtattttagatgtgtggctttcagcctttggtctaacaagatccatttgtgggcaggaaatgctaagagcatgaggttcgttg
aagtgactccttagcagcacatgaagggatgggggagggctttgatgctgggaagaaactcacctggagtgtcccatctgctctg
gccagaccacacctaggtggtcttcagcttgggatactgcttttaagtgggaatggcagtgagccaggaactgtgcaggggctgg
ggagcagggctcaggtgcagaagaaaagacatggcgtactcttgtggcttttgttggatgtccagaagggctgttttgtaagaggg
agcctccaagctgtgaaacctagtccccgctaagaagaaggaaagagcagtgattctggttattgagaaccatggaatgtatac
cctctccctagaaaagtgcttgtttgtaaaattcacatgcacagaggattcacaaaccccctgaattccatcatagagctgaactta
aatatatagaaaaatgttgacttggtgcacaaagaagtcacctcccatgggtctgtaccatggtggagtggcctgcctcagctggc
gagctttctcccctgcaaaatcctgtcaagatttggagatgaggagtccgaacagcctgggctcttccagcttaaagtctcgtatctc
ttaaaattgacagtaaaaccagagtcatttctatgttttaatgaaatcacgtggccggtggacaagaggacaaatgggtgacgtga
atatgtgtgttttcccgtagCCACCAACCGCTAAAATGCACGCCATCATCGAGCGCACGGCCAGCT
TCGTGTGCAGGCAGGGAGCACAGTTTGAGATCATGCTGAAGGCCAAGCAGGCCCGGA
ACTCCCAGTTTGACTTTCTGCGCTTCGACCACTACCTCAACCCCTACTATAAGTTCATCC
AGAAAGCCATGAAAGAGGGACGCTACACTGTCCTGGCAGAAAACAAAAGTGACGAGAA
AAAAAgtaggtcccactgcgtctgttccgtccagactttgggcctgtgttgtgggggcggcaggctgggtggttctgggaaaagt
gtgaagatacacattcttacagatgcatggttgaaagccagactcgaatttctagaatgtgtctgaaatcctgcagctaaggcgtga
tcgttacccctgctggtgcacctttattaaatctttggttaatattttatagataaatgaaatataactaaatattgatgctgtcagaacat
aatcatctgggtgggaaatttttgccctcattttgcccacttaacatttcatagagaaaacagttatatatcctctcttggattattcaagt
accacagtgttcagggctgtatagctcaattatacatggccacaaaagtgaaaattttacttggattatctattttaagctattatttttat
aacagtgtctctattttggagttcttactgccaaagccagttagctgtattttgaataaagatggtattttgacaagtctattcatatatatg
tatatatatacacacacacacacatcttccattgaatttttttttttttaattggcgacagagtctcgctcttgtcgcccaggctggagtgc
aatggcgtgagcgcgatcttggcttactgcaacctccgcctcccgggttcaagcaattctcatgcctcagcctcccgagtagctgg
gatcgtgggcacgtgccaccacgcccggctaatttttgtatttttagtagagacaaggtttcaccatgttggccaggctggtctcaaa
ctcctgacctcaggtgatccacccgcctcggcctcccagagtgctgggattacaggcgtgagccactgtgcccggcccttccact
gaattctgttctcttcagccaaaataagtttcaaatcagttgtgtaaatcttaatgcagatctcatcttcagttttgttgtagttgtttatttctg
ttgctattattttgcttttcataaatcagtacaatttttgcccttttttaaaaaaaggaaaaaaaaggcagagagaaagaaagcataca
gagccccagaccagctggtgctcgatgctggcaaggagtcaccaaatgggcaaaggtcgcaatcctttttatctggccttcttctg
gacaacttgggtgactctagggagaaatttctaaaagtgttttttcgacagataaccaagataacttggctgcttctaagtttttgcata
attaatttgtactttttttctcaccaaacaccaaaatcttgaaatgtgattttgatttcagAATCAGGAGTCAGCTCTGACA
ATGAAGATGATGATGATGAAGAAGATGGGAATTACCTTCATCCCTCTCTCTTTGCCTCCA
AGAAGTGTAACCGCCTTGAAGAGCTGATGAAGgtttttatctcattgttgaactatatttttatgccaccacaaa
acttctgctaatgtaattttggaaaatttgaagcatgtcattcttgtgtgttacagttgtatcttattttatcatcattgaggtgtatttgcattttt
gtttttagctgggtgacaaagcatctgcttctttggtttcttacctgtctagcttataaaattcgtgagcatttgctcaggataattttaccatt
ttattacaattttactcctttgagatatttagagttccaagtagagtgttggttaagacttgaaaattgttttgttgtgcgggtgtggtggctt
acacatgtaatcctagcactttgggaggctgaggcaggtggatcacctgacatcaggagttcaagaccagctggccagcatggc
gaaaccctatcactactaaaaatacaaaaattagccaggcgtggtgggtacacctgtagttccagctactcgggaggctgaggc
acgagaattgcttgaacctagtaagtggaggttgcagtgagccaagatcacactactgcacgccagcctgggagacagagtga
gactcaaaaagaaaaaaaaattgtttagttgtgatatcatcataggattggattttataggtgatcagaatatatgcatcttcgagtcc
tatgttaccatcatagattgtttttaaataaatattttcacttctaattctcccctcatctgtgtgaagaaaccactcagcattatcttgtggtt
aattcacaccactctgccattcgcgacataaaaaacaggagtctattagatttaagcatctggttttcagcagttgtgcattgtgggtg
accttttgtgggaatgattgctgattgattggactggaaaagctattggtgattaaaaatcagaaactcctataaggaaagacagttt
caaattttgcatggggttagacattcacactttaattggtgtcaaactagtcttagttgttcgtctgtccttttcttggtagttattttggaaatt
gaaaccctgtgttcactcagttcctctgagacagccagctggggcatttggccacaactcgttaggacctccatgggtgcgtgcat
gtgtgtgttttttctaaggcatgtacactgagtcctaaaggtgagccttttgcagcagaagagttctgcatggttcagaatattgaatgc
taaggctgtgtcttctctgtttccagCCCTTGAAGGTAGTGGACCCAGATCATCCCCTCGCAGCACTT
GTTCGTAAGGCACAGGCTGACAGTTCCACTCCCACCCCACACAACGCAGACGGTGCGC
CTGTGCAGCCCTCCCAGGTGGAGTACACGGCAGACTgtgagtactcactgtgtatgtcctgacctgtgttc
agctgcctgtgacagagccagctacagggctctaaaccccaagtgttctgtcctccaagtgtaacaagtatggaagcaggcggc
ccagagcctgcacatggtcccaagggagagtgccacgaggctgccctttgcttggcccagtgttggcaagatggctgccctactc
cagcattagctgtgcattccaggaaggaggaggaccggcaaaggtagctggctgcctctgcccttctttcttttttttttttttttgaggc
gaggtttcactctgtcacccaggccagagtgcagggatgcaagcatggttcacggcagcctgacctcccaaactcaaacgatcc
tcccacctcggtctctctagtaggtgggactacaagcacgataacactgggctaattttgattttttgatagggatggggtctcactat
gttgcccaggctggtctcgagctcctgggcttaagcagtcctcccgccttggccttcaaaagtgctaggattgcaggtttgagccact
gctcctggccctggctctgcccttctttaaatatccacccaagccatagctagtggcttcccttacctctcagtggctagtgtcctgtca
catggtctccccactcagcagaggcaatggggcctccgttaaacacgttgctgccctaaacaaagtcaaatgctggtaagaaca
gggagaatgggagacacatagtattgtctaacacagttgctttctttaaaaaggttcacagcaggccaggcgtggtggctaatgcc
tataatcccaacactttgggaggctgaggtgggaggatcacttacgcacaggagtttgagaccagcctggacaacatactaaaa
ccgcatcttgacaaaaaataaaaaaaaattagctgggcatggtggtgtgcctgtggtctgagctacttgggaggctgaggtgaga
ggatggcttagccccaggaggttaaggctgcagtgagctgagattgcactgccacactctagcctgggcgatagagcagtaccc
tgtctcaaaaagaagaaagaaaaagaggcttacagcataagttaacatatgcactgagaaattacatttctttttctcgctgattgc
agttcttttatggtattcattaaaggtaagtcttgaaggtccatgcaggagatcatttgaaagtgtttgacgttggttccagcgtcaggtc
tttctgtaattgttttattcagaggttaaatatggaatgaggaagctttagcagagccgaggaaccacctgctgagtctgcttcccagg
cagctctggtaccctgactccatttgtaaagcttatctccttcagttcagccggagatgaattgttaaaacatcagctcctctttatttgg
gacaagcttttgtaaacatcacagctgtgttctttgcacttcccttttagcactggcacatactaaacgtttttagactttaaaaaactag
ttactagagtgaactttctgcatgtgtccccccaaaaaccttttaaagctgagaatgtctttaaatgattaaatcaagtcatatcaaattt
cactgaatgttcaaatcagaggtcagctctactgctacagaggtgcgtgttcaatagtgtaggcagccagctgtctgaggtgctctg
tagatcactcctaacgcccagtcctcactgcatggatttttggatagacggccgcacacctttaagtcttgagccccactcggcagc
ctgtgaagctcccgccctggagtcatggggcgctgtgctctgcccaggatgcctgcccactgagggaccatccctctgcttcctcct
ttccttttccaagcctgtcgttgagtttgcttgaaccaaatgcattgtccgtgcacgtccaccagatccctgaagctgctgcaaagcag
aggactagaaattcagggcgggctgaccttgattatttgctgtgctaatcactggtggaagaacagccatgtgcagaccccgcag
gaccaggcaggatggtggagccggctggtagtggccgttctgtgacacacagcatcccctggtctggtgggaatgtgatctgaa
ctgaggcatgcaggggtggcattgtgagctgtctgggtcagaaggcttggtacattcccaagggttcaccgcagggcggccaga
gcccacacactttggtttcttcccacctgctgatggctcccgagaccactgataagccgtgacagcctctgcaggaaccctaagctt
actctgttcaggcccctgactaccaccaccctgggcactgacggcaccccaccctatcctcccaactgcaagggctctagtagg
gggtgccctctcctcccctcaatacggtgccgttgttttgaaactcatcgtctcccctcgacacagcaagagtagtggatacacaca
tgtgagagtaagggtgcctgggggctggtgaaagcatgcgtgtctgctgttagggtctgtgggttttagacatatgctcctgcatcag
ccataggggtcagagccctcctatgagcctcctcgctgagcacagcactcagggccaccaccacagtcccacccatcttggatc
tggagggtcagaaggtgggggaggtgtcctcatccagtttccaagaagagccaagagctagaactttggctctaaatcactgtaa
aacctagcagaaatcagtataaacctgtactcaggcgctcagccttatgggatgagtggctgtggcgtggcgttacgtcgggtcct
ccagcaccacgcaagcccgggcagtgcggccattccagaatctgcagaggttccagggcgcctgactcacacgcacctccct
gcctgccgtcttcctctgctaccctttgagtaccttgttctgcctgcctcatgcttctgtgtgctgttgaagtttcgtgggtgaaagtccctc
atgacctcgtcttcacttcctgggttttcagtgaagttgttgcagaatttggggtcctgtgtggcaggttgttggcagttgcaggtggag
acagcagtcattgatctacccaggttggtcatgattagggaactgcctgtaattcatggactgactactatgtggttattggtttgtaatc
agtcattgataacagcatttatttacaaatacagttcaaatagagggaacactggtcatagtttttgggttgagttccgtcatgctaaa
gttcaagataatattgttacgttcattatatgtagtttccaaaagtattaatgcagtgggatctcaactatgcttaaaataataaactgga
gagagctgtgcaaaaaatactatgaggctcagagctacctctcaaattggcatttggttgattttttttctctgcatacttttttcattttcat
gatgtttttgagtatgcatttatttacaaatacagttcaaatagagggaacactggtcatagtttttgggttgagttccgtcatgctaaa
aatctcggctcactgcagcctccacctcctgggttcaagcgagtctcctgcctcagcctcccaagtagctgggatcacaggcgctc
gtcaccacgcctggctaatttttgtatttttagtagagacggggtttcaccatgttggccaggctggtctcaaactcctgacctcaggtg
atccgcccacctcagcctcccaaagtgctgggattacaggctgagccaccgcacccagccgtgttgctattatacttaagaaaca
aagtaaaatacaaagttcatagaataactgtaacgtttgcaatgccggacagtgagggcaagagcagcccatggcttggcctga
gtttgtggcaagcccaagcctgtacagatctcccgaagttccttctcagactgttgtgaggacgtcgctgagttgcttcaagaaaag
acctaaactcatgggctctgtctgatgagcctttgtgaatgtagtgtatgaggttggtgggctattttgaaattcctgcttcagccagca
cagaggaaggtttgaggggcccctttctgtcttgggcacacccagccctgctcaggagagcttgagaagcaggtctgcggattct
gctgcccttggctgctctcggcttgctcctcgtctccgcctaatgtacccagtgtgttcacggaagtgttgtcccatggggtttcatatac
agcctgttattcctgtatctctaatgtgtgattttccatgctctgggcatgcatagctttgtttcttaaacagccagctttctacagagaag
gcacaaccgtcagaggcattgaagtaattttcagaagagggcttaaattgtgggctttgcacttgggaagtactctagtaggatact
agagagaaagctgtctggaaaattactaacattactgataacattttgggagactctcagttggggcaaacctggggccccgtgg
ggtctggaaaagggcggggctagtgtccttggagcacgtcagcttccacagcagccagttacttttcctgaggacagaggagttg
catgtgagggaggaggcgtgatttaaagcatgaagagaatcatgccccacaatgaaaccagagccctgtggcccgcgtttcag
accactgccaaccatggacaccagagacaagacaaaggacattttggccatggacttgaaacgtcagctgtatgagagcggg
cgggggatggcgcggtctccctggtacttactgggcaggtgcgtatcgtcaggagctcctcaccctgccctgtgagaactttcgtat
gtgtgtctctgccatctcctcctcctcccattcctgacctgttgagccaggggtggattggcaggcctataaggcgcctttcacattga
gggtcttaggatttgcagtccagctttgcagggagcggcagtttgtcatttgtaggaggaaatttcacgatcataaagcacggcatg
catcctgagagccaggcagcgacgctgcctgcactgccccaccgctcagagggccacaggagcagggcttcctccttgcctct
gagcagtggagccaaggctggaggtgggcgcagctccatgttctcgggggatttcttcactgtgtttcttgggggctcaccgactgc
agccgtattcctggagagagaaggaggcctgtcacagcatctgtgacagcccggaaggaaacagcagtccatacagtcccct
caggacaggcacagaggactccaccctggagtcacaggcttggtgaggtgggggacaggcaggggtgggccccgaggtgt
gcagagtgtgtgttcaggcttgtcttcctgccgcagcgcagcagccctcccatgcctgggtcctggcacctgcccctccactcccca
tgcagcttcatcctccagggcgtggtctccagatgacttacctcctagatacagacaagaccccaaacacacacatgggagccc
tgagcccaccctggggcagggtgacacatgggagcaggtcagtgccctgtgtgtggcttgccaccatcatttgggaactattcttct
gtcctaggtgagtgcccaccctgtggcactgagacccaacagctcaggtgacagtgacacctgcagcggaggctggggaagc
atcagagcctctgctgtggtggacgccaggtggcccctggcacagagagcgtgttcatcgctggctcctgccgccctcgaggact
tgaaggctgacgttgggctgggtgtggctcgtacataggacagggcccacacactggattcacgtttttcctcacaacttagaata
gcaaagttacagactttggattcttacgaagacaagaatgaatgtcttggctaaccatgatcttacccaagaggacttaaaatgaa
tgtgcaggagaaatgagaagaggacttaagatcaaaaagagagtgactaggaggtcagagaggcagccgcccaccccacg
cactccctgcttgtaagccggggccgcattgttgtgttaccacacttctgttttagagcctgttacggttttgagttacacagacatgtgt
gggcttgtgcatgtttgaatgccctgtggacccggagctctgtgaggcaaaggctgggactgtcttactagccagcgtgctccttgc
acctcgatccaaggggccacggggctcccaggaacattcaccgagtaacttcagaaaagtgaagagcagaagttccaaaag
cacctggtgcttcctgggagaagtcacctgcacaggtaccttggatccaactgacaggtgagatgaacgagctctccctgcgtgc
gcacgtctacgtacgctcgtgtatgctgaggagcaggcattggaacatgacggagctgctgctgctgcagccgcagataccatct
cagccggcatggcgcatgggggtggggtggggcagtgagggggggcccgctccgagagacagacaggtcaggccggaag
cgactgtccgtgaaggtgacgctcataccgtaaccttagcagcaggctgttgccacacagtcacaaaagtgggaggcagcagc
agagcaatgtggccacaggcactcggactccagagagctggcgagaggctttcctggctgaaagcagtgacaagtatctgggt
ctgggggacaagggaaacttggaaatgacagaaaagcccaaaaatcaagtccccacaacctcccccagcgatgagagccg
tcgtagccatgggctgtgcatcatccctacacgcccgcgcctgaagatttatgcgcgctttctgacgaacagcctttgcagttgggct
ttgttgtggctggatgactctgagccctttgctttgcttctctaggcctcagttttcccatttgtaccattagggtattaatttaaataacaga
agcactctatcgtattctgaatgggacaccagttaattctggaacattttggaggtttcccattgtttcctgtgaaccccagagagagtt
tgagaaacagatgataaagggaagacaagatcgtaaagtgtgatactgccatcgaaggtctcgagcctcatagttggcgcttta
agcaaaataggcggttaaaaacaggtctacacatgctgtgtgtggacccaaaccatgaacacatgctgggccccagcccgtct
gttgctgttcccttggtcttggcgtcctgtggtcctcacgtgagctgcacgcagcgagcagagccctgacttccagtctggatttctgta
aagtgatgccgggcttatattatttcaaggacttcatggttacttctccctcctggagttgctctatggctttttaaagcagctgactttttat
ccatcttctcaaagtattcagcttcattttcacagaaatgataattctcatctctcactcaaattttatgtttgcataaattttcatcaaacac
ataattacagtaagtttaactggaaaaaataagagagactctactgttaaaagcaaaaaggcccaggcttctgaagagacgcg
ccttctcccctggtgtttgtcatggcaccagccaacacagcaagatggagccaccagtccagccagggagcttctgcagtgtttca
aacaaggcggcaccagcacagaaatccacaggccctgcagtgggaagggatgaatgagtccccaaaaacagatccaaata
aaaataaggacggagaaaaggaaacaccaagctaacaggaaggggtgttctaagacacaaagagctatttggaaaacatc
acataagagcctcactttcacccagtatcagaatcacttccagatgtaaatgccagttgtggaaattcttaggaaagtaaggatgct
tcagcagaatgaagaatcatctattttcagccagcagttatattcaattaattttttaaaaaatgaggggaaaaaaaaagcctgtatg
tctaaacagcttttacaatcaaataggaaaaaattctgatagtctagtagaaaaaagatctgcagtgcgaataattcagaggcaa
aaatgcccatacggtttttaaagactcaccctcactgcctgctcacaggcatcgtgtccgccgaccgtagaacctgaaatccgtag
taaacacccctccagcgctttggttcagctcagctccagcgaatgttaggatgtgaggcttcgtgttacagtagaaaggagcgcac
tcataggcatacagaacactgtgcaggtctaagacttaggagaagacggagatttctaggctgttggaatgttattttgtatatgcga
gtttgggtagcttaataatagagattaaataaaagaaatgcagacaaaacctacaatggagtgccatttttcagctgtcactcaag
cagagaaaaggtgacagacgtttattgtcggtgggggtgtagatttgtgccagcgatgtggtacttgtggagggccagcgggcag
tctatcagtctaaaacacacgtgcctccaacccaggatgtctgctctcatttactagtatatgggtgaacatagccacatagatactc
atcatagtacagttttagtagtagcaaaagactgaggcgacgcacatcctcggtggaggactaatggagtccctctggtgtgctct
cgcggtgggctgcagagctctgggggtgggccagggcccgagcgctgcagggccacccaccggaatggctcttcctgagggt
ggaggcgaggtgactcaaggggagggcagcacgaggcagtctgttcacgtcagaaggggagggaagtgtctgtagtgcttct
gtatcggcacctaaaagggcaggtaagaaggaccagtggccagcacagagaggggaactggaaggacgaggtcgcagac
aggagtggctgactcacctgcccgtttgcgctgcttgcattttttcagcgtgactcgtcaccctctcaaaacatgcagattgacgtggt
acttattttgagactaaactggtatcctgagttaattccttacaactctgtattttaataatacgtgcttttatcattttgtttcatcagctcagt
gtgtttttgtttaggcggaagtggcccccgtaacatctttcccccgtagagacttgcatacccagtactgctgtgtctcgtgggaggct
gctgggtcactaagcttttctggctttcatacgctgggtatttaataatcaccttaggatatactcagtcgttctttcttatttacttcctactg
atggagattttcctacattttggcagcctgggaagaaaagcatctatttttttccttaaagtccagcagaattttatatatatataaaatat
gtgtataaaataatttttctagaagctcattaaatatagatgtgtaaaactaacaatttatttcatttattaattttcctgacaatgaactact
tttcatgctctttatttttcattacttgtcttctgctatttagctataaatccttatagaagagtataaataaataaaagtaattatgcaggag
gcagtcatagtgaaatgctgcccactgtgatagcctgtgggttttttttaataaaatgccaactcagtttttcttaatattctataaatatct
gaagtgaaattaaaccattgcgcatggctactatagatattttcttgcttctatccctgttttttaaatgtgcctttgctgtttatggtatattttt
cctgcacatcatgactttgaagttctttacttactttccaaacccatttttaaaaatggttttattgtatgtcaagagaagaaggaagaa
agcaaaaatgttacccaggattccaccatccagaaatagccattagtgaacatcattagcaaagtggaaaacactgaggtcatc
gtgctaaataaaaaggaaagaaagaagcttgttatctgtggaacacaaagatcatctttatgcacgaatatcaattaaaatgttgg
atgtgtctctagaaatacttacgttaaagtggaaataaacttaattttacttaaacagaagagcctgcaatctaaaaatgaagtaact
gtcgaacttcggatgaaagtttcttttatgcctaaagaattcagttctgaaaaaaggttaggagaacattgagaggttgtcattgtag
atattttttaaagctgtatttttcctgatttttgttagaattattcaactttttctttgcatttaatatatctcgaacatctttgtattaacagtgca
tgtgtatctctctcttctagctgtagcaactacctagtatcctgttgtatagatttattgtgaaacagccctccattaattgataatttgattat
ttgtgacttccaattatttctacttttccagtgctgtaatgaacattattcttttttttttttttttttttttttgagacagggtctctcgctctgtcacct
aggctggagtccagtagcgtgatcttggctcactgcaacctcaagctatcctcccacctcagcctcctgaccagctgggaccaca
ggcatgtgccaccacacctggctgactttttagataaatttttagagggtcttgctatgttgcccaggctgatgttaaactcctgggctc
aagtgatccacccaccttggcctcccaaagtgctgggattacaggcatgagccactgtgccctgccaattatttattacttttaagca
ctaatgtggtataattctatatgcaagataaaaatcttagaaaataaacagctaggtcaaagagtatgtgcatttgtttaaagtattac
cagtgactgagcagttgccttctgaaacattgtgtcaatatgcattgccaccgctaggatatgagtgcttcttagttttgtaaccatttaa
aatgatttgaatatgctgatatagaaatatatttaagagtgagtaggacagtcaatatactatatttatgactttggttcctttaaagtatagaa
atattatttttatattatgagagtttataaatagtatttgcattctattattccccagttgcttttttttttttttttttttttttttttggatggagt
cttgctctgtctcccaggctggagtgcagtggcgcaatctcggctcactgcaagctctgcctcctgggttcacgccattcttctgcctcag
cctcccgagcagctgggagtacaggcgcccgccaccatgcccgattaattttttgtatttttagtagacagggtttcactgtactagc
caggatggtctcaatctcctgacctcgtgattcacctgccttggcctcccaaagtgctgggattacaggcatgagccactgcgccc
agcccccagttgcttacttttagttttatggttgactggattcgttttttccctacagcttctccttttgagttatttattcgattcatcttttcttgat
catttgaatttcataaacaagtacgtttttacaagggctgtagttcatgaattctgcctgtcgaaaaatgcctgcctttgacctttatgtga
acaggacggcttgtctgggtataaaactcttgggtaatgctgtgggcctctcagatctctgggaactagaatctgacactgtcctttcc
actgcagggtggcccggggtagctggatgggtcatgactacctatttgggtttgttcattggttggtttgctttcattgctcctatgctgct
ctctgagttttttcacgcttgaaaatctttctcagctgccttttaacataaatagcaatttgcagggaacgatgcaccgagtcgcttgcct
aggattcagaagtggaattgaacgttgctgtggaaagggcgaggccagccctgtcttccctacctgccctgggaggtggatgctc
tttctgacccaacacatctgaggaagtcttgctttacccttaacttttattaacttaattgcccatttttcatggactatattgtatacctttca
gtcagagattcagttctcatttctgggaagttcttttctgttgtgcctcgagcacctttcctgtcccacgcattggtgcctctgcttgaagg
acacagtctcttggttcgggtcacctttcttcgagcctgtgctccctgtgtttctctttggcactcagcaggactgtgtccatcttccctgtc
agcgacttttttcagccatgtctatttattccttgtagtttaaattccattggttttgcagtggtattgtttggatccttggctggttttctaagcttt
acagcagggccccacagcctttctatataaaggtgcatgtcttcagccctgcagctgcccagcctctatgcagcccctgctctgtcc
ctggggtgtggaggcagctgcagatggttgtagacccatgagtgtggcagggcttcactaaagcttaattgatggatacgaggcttttca
tttctttttttttcttttttttcttttttttttttttttttttttttgagatagagtctcactctgttgcctaagctggagtgcagtggcacgatctca
gcttactgcaacctccacctcctgggttcaagcaattctctgcctcagcctctcgagtagctgggattacaggcgtccgacaccacgctt
ggctaatttttgtatttttagtagagacagggtttcaccatcttggccaggctgggaactcctgacctcgtgatccacctgcctcggcc
cctcaaagtgctgggattacaggcgcaagccaccgtgcccagcctagggttttaatttcatacaattttgatgtcataaaatattatttt
gtttgtttgtattttttcaaccatttaaaaatgtaaaaaccaggccagccatggtggctcacacctgtagtcccagcactttgggaggc
caaggtgggtggatctcttgagctcaggaggtcaagaccaggctgggcaacatagtgagactgtctctacaaaaaaaattaag
aaaattaagtgaccataatggcgcacacctgtggtcccagctgctggggagagtgaggtgtgggggttggcagaggggaggg
catcgaggagttccatgctgcagggagcaatggttgtgccactgtactccagcctgggcaacacagcgagactctgtccaaaaa
aaaaaaaaaaacagtaaacaccattctgctgatggctgtacaagaacagggagagcgcctgctggaccctgcctcacagcct
ccccctctgttgcatttggttatgttaccttatttttgtgctttgttgaattcctgtcttcccagattcatctgtggctcagagagctcaaaggtt
cctcgggtcacatgctcctgtagcctgagatgccattcacatgccatgctacttccctccgctgcttttcctgggggcgtgtgcagggt
ctcatgccgtctggtgctccttcttccctggtgtgcaagcctgtgtgttcttggtgtgggtggattctccttgatgctctctcaccttctcttag
cacctttttcttcccttccaacagccttcttgggaagacctatcctctgctgtcttttgtgagattctaaaaatgtcctagattggatttccttc
ccccagtgagggaactacagggagagacgttcttgagtatcacagcatatgtgtcaggcagggccccaggtccacaagcccc
gttctcctcactgtcaggatccccacggcaggtcattggcatttccacctgcttctttccatggtggggcccaggtctcacttcagcca
cttgctctctttacccacaactctctggaacctatttttatgtaagaagtcttcaaaacctcagtacagcattaaaaattgaaagcttttta
ctttgagggtcactgatgaaaatggtaagttatgtttagagacaggcttttttttttctagaggaaagttttatttgccagaaagaggtga
cttttaagcacagtgggctaaaattccaaatagctggttaaatgcccaaaacggattcattttggtagtttcccagtttgacaaatgag
taatcttgcatcactacagaaatcattcaggtttccctaatccaatttggtgatgtcaaaacaagtcttctcttgttgggggacttttttttttt
tttaagatactaggtcgtcgggaggttacaacaaaatacagtgtgttgtgatggactgcatgttaagtgattttattgtaagtcttggca
tataagaacccattaacagatcattggaaaccattctgtgttgtgatatggatagcctcatggtttatattagtctgttttcacactgctga
taaagacatacccgagactgaggaggagaagaagaggtttaatggacttacagttccacatggctggggaggcctcacaatca
tgacagaaggcaaggaggagcaagtcatgtcttacatggatggcagctggcaaagagtttgtgcagagagactcctgtttttgag
actatcagatctcataagactcattcactattataagaataatgcgggaaagacccgcccccataattcagtcacctcccaccagg
ttcctcccacaacatgtgggaatagtggtagttataattcaagatgagatttgggtggggacatggccaaaccatatcatcccctct
cacccctcccaagtctcacatcctcacatttcaaaaccagtcatgctttcccagcagtcccccaaagtcttaactcatttcagcatta
actcaaagtccacagtccaacatctcatctgagacaaggcaagttccttctgcctaccagcctataaaatcaaaagcaagttagtt
gttttctaaatataatgggggtacaggcattgggtaaatacaaccgtccatatgagagaaattggccaaaacagaggggctgca
caggccctgtacaagtccaaaatctagcaaggcagtcaaatcataaagctccaaaatgacctttgactccatgtctcgcatccag
gtcacgctgatgcaagaggtgtgttcccatggtcttgggcagctccgcgcctgtggctctgcagggtacaacctccctcccggctg
ctttcacaggctggtgttgagtgtggcttttccaggagcacggtgcaagctgttggtggatctaccattctggggtctggaggatggtg
gccctcttctcacagctgcactaggcagtaccccagtagggactctctgtgggggctccgacctcacatttcccctccacactgccc
tagcaaaggttctcgatgagggccctgcccctgccacaaacttctgcctgggcatccaggcatttccatacatcctgtgaaatctag
atggaggttcccaaacctcagttcttgacttctgggcacttgcaggctcaacaccacatggaagctgccaaggcttagggcttcca
ccctctgaagccacagcctaagctgtaccttggccccttttagtcatggctggagcagctgggacacagggtaccaagtccctag
gctgcacacggcacagggaccctgggcccagaccacgaaaccgttttttcttcctaggcctccaggcctgtgatgggaggggct
gccatgaagacctctgacatgttctagagacattttctgcattgtcttggggattcacattcggctcctggttacttatgcaaatttctgca
gccagcctgaatttctcctcagaaaatgagatattcttttctattgtcagactgcaaattttccagacctttatgctgtgtttccttataaaa
ctgaatgcctttaacagcacccaagtcacctctcaaatgcattgctgcttagaaatttctttcaccagataccctaaatcatctctctca
agtttaaagttccacagatctctagggcaggggcagaatgccaccagtgtttttgccaaaacataagaagtcacctttgcctcagtt
cccaacaagttcctcatctccatctaagaccacctcagcctggaccttattgttcctgtcactatcagcattttgggcaaagccattca
gcaaatctctgggaagttccaaactttccctaattttcctgtcttcttttgagccctccaaactgttctaacctctgcctgttacccagttcc
aaagtcacttccacattctggggttatcttttcagcagtaccccaattctggtaccaaattactggattagtccattttcacactgctgat
aaagacatacctgagactggagagaaaaagaggtttaatggacttaacagttccacatggctggggaggcctcacaatcatgg
cagaaggcaaggaggagcaaagtcatgtcttacatggatggcagcaggcaaagagaggctgtacagagaagctcctgtttttg
aaactatcagatcttgtgggactcattcattaacatgagaacagcgcaggaaagacccacccccataattctgtcacctcctacca
ggctcctcccacaacacatgggaattgtgctagttactaatcaagatgagatgtgggtggggacacagccaaaccataccagttt
tatctcagttggaaaatacttggacacaatgtgtgatgagccaaataataaatgcttttaagtatttgggaggatgggaaggaagat
catattttcttaaaaactttgggcttacatcttaaggagtttttggtttgttttaccatttttattcttgcaatatgagatttatgttatagagagct
agtagataaccaccctgcctaaaacgaacaattgccagaagggatcttttaggaattcttgaaaattatctgagttcaggagatga
agtcagaagtcatgagaatggagataattgagtggaaaagagaaacttgcagaaggagaaagagtttctccgccctgatttctct
cattcacttctagaggaccttgaaggtttctaacatcccctgggtgtattaggcactttcctcattcttgagaatgcagaattcagtaat
aaaaacaattattcttgaatcgtgttgtcagtgcctgacatttacatgcatagaatgtggacctctcctggggtgcaggtcttcactgtg
aataaggcagcactctaactataggcagagtaagattctcaaatcaggcaggctggcacagtctgaaggacctaaaaatacct
gtttcagggatctgcatcttcagatggtaatgaaacttttagtaaggctttttttttttttggcaaaaaaaaaaaggtagtattgtagaattt
tacattaaatagtggaattgccatgaaaacaatttattctgacattgatccagcagccgaataagcctgcagggaatggcgactctt
ggcagcgggtcaggctgtgggcttcagagtgggccgcttcctggcttaccagccctgctagggtaaatctgctctcagcggcttcct
cctagaatccagcttgaaaattaaatggaaataaacaaacatcggttatggtctgggaaatttgctacatattgcatgttctgtatga
cacactaatacatgtacatgcgtagtattacagatgactgcatatattggtgaaccattagcctgaattggagaggagatctcaggt
gagtattgggaacatgcacatcatctttgcagtgcagcccaccttgtatctctgagaagtcagtgtgcatgtggagaaagaatgga
agggaatgcaggcaagttaagatcacccttgagaggtggttctcagatggagctgtgccgccttcctagctgaggacctacagc
gttgttgcagtgtagactcatgtaatggtgccatcttttaagcaagtcttgacttttgatgcctcatttgctgctgctagacccaggcgga
gcaagcttctctggcatgtgggtcgtttgtttgcagtgtgcatttggtgaaattgacagctgggtttccctgtcccccgtccccgcctgg
aacatcactgttctgagcctgtagccagtgcttttctgtgacttctctttctttcctgtgttcattcctgttcttgttgcttgtatgttacttctgtat
tttgctggagcacatcctccagtagtttcccaagaaagggtacataggaacacaaagttttttaaattcttggatatctgaaaatgcct
taattttgccttcccatttgacaggtagtttggaagcacctagaattgcggggtgggtatgactttccctaagaatgtgggtgctggat
gccgccatctgcaggagcctttgctgccatggagaagctcatgctggccgggcatgctggctcacgcctataatcccagcactttg
ggaggccaaggcgggcagatcatgaggtcaggagatcgagaccatcctggctaacacggtgaaacccggtctctactaaaa
atacaaaaaattagccgggtgtggtggcgggtgcctgtagtcccagctactcgggaggctgaagcaggagaatggcatcaacc
cgggaggcagagcttgcagtgagccgagatcgcaccactgcactccagcctgggcaacagagcgagactctgtctcaaaaa
aaaaaaaaaaagctcatgccatgctcgttaccattctctcctgtgtaacttgtacaggtgttgagcgatttgcatcatgctctgccatg
ccaggaacacagtggacaaacctccttctgccatggagcttatgttctggagggcagagccagacagtgacagtggacatgtg
actaagagcgatggagaaagtggccatgacaaggggaccagggttctggggaggccagcagtgctggcatcatggggaca
gggaggcctcctgtgaccagagaccagaggaagtgcaggtgagcccagcaattaccatgtgcaggatgggggatgggacag
caatgggacatgaggtaggagaagcaggcaggtgggtttgcaggagggcttcccagacaagggacttagcttgaccctggttg
agagaggtcgccatgggagggattcaggcagaaactgtggaggcaagggtggaaaacccggggcaggcagcagccaag
aggctgtacacatggagggcaggaggtgctgcagctggagggcaggactcagagctgaatcatcgggcgtcagccttggggt
ctgccagatgaactggatggatgaggggtgtggccacctcctgcattgggggactacagaggagaggcatggggagaaatca
ggggctctgttctggacacattcggcttgaaatatgtacgagacatcccagtgggaatgttgagtaggtggttaatgcacaagttca
agttcagctcagggctggagaagtgaattttgcagccatcaagtataaatagaattcaaagccactgaacttagaagagttcctgt
caacaggatttagatccaggaaaagagaccgagaggcatggccgctgccgaggaagagcctggggctgtgggagcagcga
ggccaccatctgactctggatgcctggagagccgggagacaggaaggctggcttgttcctgcctctcagatgtgctcagctagtta
catttgcctggctaaaacacaggggccatctctttaacatttcttattaaaataggtgtgtgttttcagaatatctatacttatctccatag
aactcttaactattttaattctttttttttttttttgagacagtctggctctgatctcagctcgctgcagcctccacctcccgggttcaagtgatt
ctcctgccacagcctcccaagtagctaatatttttttgtattttttagtagagacggagtttcaccatgttggccaggctggtctggaact
cctggcctcaagtgatcccccgaccttggcctcccaaagtgctgggatgacaggtgtaagccaccctggccagcctattttcattct
taatatacacattgttcatcctccctgacttagctcttccagaaaggtggttgctcaccaatctcctctctaagaaccttctcagcacag
gagttctgttctgtgtgttaaattcacacgagattaagatcatgcagagatacgagagaactggctctgatttttgcaagaagccagt
tgaatagagggccttgggagataattaggcagatttctctgacctatgttaagtagctctgcacgtttcagaggaggcagtattgga
gaaggacttacaaatgtgcttcctgcttttaagcagcttggttctcgtcatacaactatacttgcctttagggactgtgtaggtacctatt
ggaatttctttcttggatttatttggagtaggctttcgtagtactcatagcgtttattagagtaacattacgtcagcatttaacttagtttaaa
acgtagtcccctttgggaaattcaatataaaatcctaagaacagcaacaaacctaacaagatatatgtggtcccagcttactgag
ggttcaactcgatgatggtgcacatgcaatttgcattcagtagaacatcagtaaaatgcttgagatactaaaaactttattataaaat
aggcttcgtgttagatgatgtcatccagccgtcagctactgtaggtgccctgagcacatttaaggcaggtgaggctgtgccatggtg
ttcggtgggttataggtggattctgtgcattttccacttcacggtgttgtcagtgtatggtggggttgtcgggaagtagcctcgctataag
cccaggagaatcccggcatgtcgtggcagcctgaggacagcaggagccccttggcacactgtgccctcccccgttcatgacta
gtaatggcacagttattgtaaagctgatgtggcttttgccagcccagacttcagtttgtagactacagcccagcttgtagattttatttct
gttgtcaccctgtactagtccagaaattcttaaaatttagtgttcacgagaattgctgtgtacaacatacaaggggctgtatacaaaat
ccctgtgtcctatagttggtagtcagtttaaagggcttcagtccagttaaagggttctgtgagctgtatggtgccaccattgtgtgcggc
acgtgtcaagcagcttcatggtcactgcaggatattttagcactgaggcattttagaagcagtccaggccgtgccaccagctggca
tgaactcactcattaaacacttactgagggcctgctcatgccaggagctgtgtgagcggctaaggttttgtggtcactatttggagat
atggagtccttgggaacatagctgcacaccagtcctagtggcgcaggagttgccatagggcgttgtttacagggtccccacgcga
gcccagagcaaaggcctcctgagtctgccaaggaggcagaggcttcccgaaagaggtggcactggagataagctgaatagg
ggcctcatggcaggcagagaccctgtgaggccgtgcaaaggacagagacctggggaacagaagccagggcaaggggtgg
gctgggcaagcggcagaagcccttcgggaggctggccctgggctgctccagatgacttgtgcccgtcctgcctcccaccaggg
ccacagtgtctggggaaggatggatctgacgtcctcccttagatcttcacatccctgacaccctatgaagtgagaatctgggagaa
gcaacccaggaacggtgtagcggaattcatgaaccactgtggtgttggttcccgggctgcctccgagcatggcagtgccatagg
acacgtcccacattctctgtcggcagacagagaagtgttttcatctcatcaagcaacacatactttatttctcttggagtccttttgaga
gacaggatgattttcaaatttgattaaaaccttggagagaatcacaggtgtgtgtggggaagaggtgacagcagcagtggctagc
agcagaccgcctcacagaggctgcgcgtgtctcggcttcacagctctcctctgtgagaatctcctgggtctgggtcaagggtgtgc
ccagagcattgtcagcctgagtggtttttagcgtggagcctctgaagcaagttgtggacctaggctaggatgtccctggagtgttttc
agatttgggcatttgtttcatttttacacctacaggcagcctttttttctttttgtgagctcagctcaggggctcactccatcacccaagctg
gagcacaatggtgccatcaacacctcactgcagccccaaactcctgggctcaagccatcctcctgccacagcctcccaagtag
ctgggactgtagatgtgtaccatgcccagctagtttattttattttattttttggagatggggtctgatggtattgcccaggcctgaagcat
cctcccacctcagcctaccaaagtgctgggattataggtgtgacccatggcacccagcctaaatttttcaaattagctgacatttttg
acatttgtagtggatgagtctctgagcagtctgccattttgccggcactgctattttttttaacacttcgtttttatttaacaagatggaagg
ctcaggaaggtcatatagactaacagtctgcgtgttctttaaaggaatggcgctcagctttgaaaacagtttcttcatctctgttgtgttc
cagtgtgattgcactttacacagttacataaagaatgcaggtatcaggttggagctgcataatatgtactactagttgaaataattata
aaccgttttgttttgtttgtttttgtgaattcagatcccgtcctttgtggccccagtttaaaacatgtttggacactttttaggggtgagactga
ctgtccagagcaggacatggggtttccgtccttcctgctgaggtgggaggctggagacctgacagtagccagtcggtagtggggt
cagttccgcctggccctccccagagctaagcacacactgggctgcactctctcccctggagtgctggcttcgccctggctgagag
gaagcatccatacatagtagcctgatggctccagcagggagtgggtggaagcagcagctccccccttccagggatgacgttgtc
tcttacagaagcacatgcttatattcggattcctgattttgataggaagcctatgttggaccatcaggtcagttcgttggtccagcacat
actctgctcaatgcagaggctgcagagacagtgaagacaggacctgccgctgcaggagcctcagagacggtgcctgccctgc
tgtcagcctcccattgacatccaagggtctcatcccctgctcccggccttttctcagaaatgttgctcagatatatctgtgttgacgata
atgtggagcacatcgaacccacttatcttattttgaaaatttggagtattactgtttctgtgtcatggtggttggtgtgtaacatggagcta
gagaacaacggtttaggagttcatcatgtataattaatttaaataagtcattagcagctggggaatatgcctacagcacataggaat
tatgctgcctcgccaatctaagatggaaaggtcaagatagtctaagttgtacttctgaaatttttctctgcatagcatacattactggaa
accatagttaagcttttactgtttttcaatgttattgttttaaggtgaattgattgaaagtgaagataaaagttcttaattcgaaaaatattttt
gccatctcctaataaagaggaaattaaatctctgtgtagtcagaactacttgcttatctacaacaggactggaaattaaatttcgtaat
taatcattgaatcttctgtgattcgtggttctgaacatttaaccccaaaaaggataaatgtacaggatttttaattgttaagacagcgtg
cctctaccctacagatacctgcttgtgtgcacagcataggtggcaagacggcatacatcactgtctgtgatggaaaggtccagac
acagcctcagtgcccctgggaacttttatttactgaataaattcctgcacagcctgtgttgctggggccgggggctgacgccagggt
tgccaggagcagctgccttactgaggggatggtttccggattaacgtgtgaatggagggagcagcgtgcctggggaatgaaag
caggtgtcagcgcggggagctagccaaaggcatttcctcacatgtgcatttaggagcataggtggccttcgtgggccgtgtgagc
aaagggatgactggttgccgctagagaggagactgttcccaacctgcacattttgaagttaaggaggacattaatttgtctagaga
gtattcatatctggtgcctttgaatgtcctcatgccattcgctttccatctgtctttggatgcgtgttgtggctttgcctggttcttttaaattgca
tattgtgcagacaactttttgtatcagaaaaatctagaaaacagcatggttggaagtgagcagaggcaaggctgcatcttgccgg
gggaagggctcttgtggctgcattgtggactcatggaccagcctgtggccggccatgctcactccggggcaatgtgtctccacag
CGACCGTGGCAGCCATGTATTACAGCTACTACATGCTACCGGACGGCACTTACTGCCT
GGCGCCGCCCCCTCCCGGAATCGACGTGACTACTTACTACAGCACCCTTCCTGCTGGC
GTGACCGTGTCTAACTCCCCTGGAGTGACGACCACCGCCCCACCACCTCCTGGGACCA
CACCACTACCGCCCCCAACCACAGCAGAGACTAGCAGCGGGGCCACCTCCACAACCA
CCACCACAAGgtaggtgcagcgtccaccgctgcctgctgtgtgagtcactcagcactgcagtcactggggccgtctgtgtct
ccatggggggcttgtaatctagatcatatacaggggtccccattgtctgagtagttattattccaaatccccaagttacaaagttgac
aggaaaacagaaatggttgtagcacaaactttttagcattgaagttaaaccacttataaagttgaattcatttcacgtcgcacgctg
gccccagatctccagcatctgttcttgcgctttgtgtcagagtctcagttgagctgtgctaggcaaaatcagtatgcagtgaagctgc
agttgtttgcaaaacatgcaggttcataaagttgacgcaggtgatgttggggtgcttcatgagtctctcccaagctgttggccaccag
gggaccctggcagctactttagttaacctgtgaagccatcggcagagccctagcttctccagcagcgagggcccccagtgttcag
gggacgagtatgagacaggcgctttaccagtgggcctggaatgccctgccttgaaaggagactcctgggaaatggaatgaaac
acgcgagtttctgtgaaaacgactctttctggtcatgctgagcaagtcagacaggaaatgaaggaggttgaaccatgcttgccga
cttgttttcaatataacaacaacaacaacaaactgcttattctttgttatttctaagaattagcttgtgattggggggaaatgttaattagt
aggaaaaatgcaccttttatcactaaaatccccatttttcactcttgacaacaatcctgtctagttgactttagtttctgtcgtgtgcatca
ccttcaacaagagcctcccctaacacactgtttataactcacatgtctctccgggcatctgaggcggtgaggacccccgagcagc
caggactgagcttggcgagcccctgaagcccaggggtctcacagactcttctcctgcagTGCACTTGCCCCCGTGG
CCGCCATCATCCCCCCGCCCCCCGACGTCCAGCCCGTGATTGACAAGCTGGCCGAGT
ATGTCGCCAGGAACGGCCTGAAGTTCGAGACCAGTGTTCGTGCCAAGAATGATCAAAG
gtcagaagaagaattttatatgttaggtatatggcatttgggggtttcgtttagcctttttttaaaaaaatgtaggtacagaattaattttttt
atatatttttaagccttttcttggctcaaatgtcttttttttttttttttttttttttgagatggagtcttgctctgtcacccaggctggagtgcagtgg
cgcgatcttgattgactgtaacctctacctcctgggctcaagcaattctcctgtctcagcctcctgagtagctgggactacaggcgcg
caccaccacgcctggttaatttttgtattttggtagagacagggtttcactgtgttggccaggctggtctcaaactcctgacctcaagtg
atccacttgcctcagcctcccaaagtgctaggattacagatgtgagccaccacgcccaaccaatgtctttagataaatacatttttta
attggcttgttaaattgcttagacttgggtggtgtttttaaattatgttacctgttttttgtttcattttttaagtaggaattttgaagctacctaaa
ataaaagcctataattcatggttttcaagaatctgccttaaaaatctagacacaaacccttctttttaaaaaccaagcaatgtcccac
gcctcagtactaataaaacgtaaagatatgttgtcacatttgcagcgtgacctgtgtaaccccgggcaagcgatttcgaccccctgt
gtgcagtctccctcgtctataagatgagtagctaaaacagtaaccaccttgtgggattgttgagatcagtaaagagctaggagaac
agggcctgttgttacttcagtgagcttgtcttggtaaatgacccattttctttctttttctgctcagATTTGAGTTCCTGCAGCC
GTGGCACCAGTATAATGCTTATTATGAGTTTAAGAAGCAGTTCTTCCTCCAGAAAGAAG
GGGGCGATAGCATGCAGgtacgtgtctgaatgcagggaggctgtgaagctcttagaggtggctccgccttccagatc
agaagtcgctttctgtttcttctcctacaggtgaaagggctgggtgattcttcacctttttttaatgtgtgtctggcatactccatctttcacgt
cccccttagctctggaacctgatctgttgaaagcatctgcccacgttcacagcattgatgattgtttgtccagcacgttctaaacaaac
aaaaaaaatcctgttccttcaactgttcgatgttttggccgtctacagttactagctacctttcatgacagccgggtaccttgcttctgttg
tgttaacatgtatgaaatatataaaatataagtgggcgcctcatgcctggccagctggtgctgggggtgtcctgcagcacggcctct
gcctgtgcctgcacgcccttccccctcaccagatccccagcgtggtgctggcgcacttggaagtgctttttgtcctacagccccctctt
ctgcctttgctctgctcttctcagttatatagacaccctgacatttttgtaaagccagttttggtgaggagatgacatgggccttacttctc
aggagatttcttcagacccttatctccaatagcccacactgaaagaaactgactcctctgtaggtgatggggataatttggtattttta
aagaattctgagtaatcagtgtccaaagaaaagatactgaaaattggttcccaaggcagtattagggcttcaaagagtatagtgttt
tttcagacaggagaaaatcttccattcctctttgatacattccattgtaagaaaaaacagcagatctggatttggaagtctgttcccag
tgctgcttgggcagtaatgtacaattgccgttgtccagtgaaacatataccgtatacatctctctttttttaaaatttctgtataatttcctgc
tgacagtttatagtgacatttaatctctagGCTGTGTCTGCACCAGAAGAGGCTCCCACAGACTCTGCT
CCCGAGAAGCCAAGTGATGCTGGGGAGGATGGCGCGCCTGAAGACGCAGCCGAGGT
GGGAGCACGGGCAGGCTCAGGCGGGAAGAAGGAGGCATCGTCCAGTAAGACCGTCC
CGGACGGGAAGCTGGTGAAAGgtatgctgccacttgcatgttggccttgcacattccaccataagttggcaagcgta
ggatcctcggtgacctcagactcagcgccctcacctgcaggctggggtggggttggcggccccctggaggttgctgtggtgaaa
cctctgccttccatgctgtgtcatgcttgcctcgcgtggcattggaggtaacgtgagtgtgagcagcccttaggtatgtgtctgtttaac
agtctgttcagtgtactggacatttgtacagaaagtttcaaataatcctttgtactccctgggacttctgaaactatttatatgcaaactgt
tgtaccagtgaaattcatttattaatttgtcaaagcagattccttgagaatctctaccaggcaatacttcactcactcgatttcagttactt
tgttatgttcttggagcaagactttgatgtcacaggacagacaggcatgtaaaaatacaaagtcagtgtaattaaaaagcagaca
gaagcaaaggccagagcaggcccttagccaggaacctcgtggagcagcagtgggctccccccgcgggagggaggttctgtg
gagtagaggcgttcagctggtgttgcgagaggaacgggaagctctgaggcaggggtgcagccctaggcaggagccccgtggt
gcgagctgcccggccccgtgttgagatgcggtaggtggtcagcagtgacttcgggggtggctggtgaaggagccttggccagct
tgccccggtgcaccctgtcggggaggggccagcacatctgacaggctttaggtcagcggaataactttatccagtctggtgacttt
gtgatgcggttaagccactggagcgacttcagagatttctggtggcattggtggcctggaatggagtgtgacaggtgtggcagtgg
ggtgaggtgtggcagtggggcgaggcgacagctcttgggtcagaaggaaaggcagagtggagacaagagattgaggaagt
gggctggggtgataagaggaccgtcctttgcataaagatgccttgttgtatgagaatggtgatcattcagcgaaaccaaatccatg
tggatgaaccgctaactaggcaattcactatatgtgtctttgggcctctcattcgagtaggttacctgagcacaagtgatccagctctc
acccttcccggccacccgcatactctcactgggataatcaaaggaatgtaataagtagaggaggaaaatggttactgctctaga
aacccggggagaggtactgtctataggtcagggtaaggcagcatacctggagcttgcagagaagtacccttgagactcagggc
agtgactccaggggagtcgactgtcagccacagaggggcaggcaggaggctggaacaagctgggagcttcccagaggcag
cagtacctcatcccttctcacaccccagaacacaaccacagcccgagcctgcctgctgccccagggtttgtgagcccagggaa
ggcgcctgacccagccagctatggggtgtgcagaggggttgtgagcccagggaaggcacctgacccctgccagctgtggggc
ccgcagaggagcagccctgcccacaaggctgctgccaaccagcgtgaccttctccacacttccctgattgtcctagaacctggc
agatgaaacaacacaccgagaagtttaccgtctacatccacccaagcctgagacacttgaacagagatctactcaatatctgac
aaaacccatgctaactctcagttctcaaaagcacaggccagcctctctttgaaaagatgcggagacagaaatgtcattgcgccc
acagagattccaaagttcgggagacacagctgagcctccaggcatatgggcatctctgaaacagactcttgcgtaacaggaga
aaaatcttttaagtctctaatttgtattctacaaaatggaaaatattataaaactagtgctactggtaatcagacatggaaaagattgct
tagaaattgctgtggagtgtggtggctcacgcccataatgccagcacatagggaggccagggcaggcagatcacttgagtcca
ggaattcgagaccagcctgggcaaatgacaaaaccccgtctctgctaaaaatacaaaaatgtagccgggcatggtggcacat
gcctgtagcaccagctcctcacagaggctgtgaagtgggaggatcacttaagccggggagatagaaaccagcctggacaaca
tcgtgagaccatgtctctacaaaaaattaaattaaattagccaggcatggtggcacccatctgtgctcccagctacttgggaggcta
aggtgggaggatcttttgagcccaggagacggaggttgcagtgagccaagaccacgccactgtgttccagcctgcgtgacaga
ggaagaaaattataggtatctttttaagtacacaagctacaactagaagttaacactagaaaataaagtatatgagaacatatatt
attttaaatttgaatgaatttgaaaatcagtgaaaaatatcgctttctaagaaaacataagtgctgaaattatttcatgaagaaatcag
gaaccaacatagacctatagacctgaaagaattttgaaaataattggttagtgaaatacctctcaacccaggcagtcagccaag
acaaattaaaggtcaagttaattcaccctcaagaaatagaatgcctacatcattaaagccactccagagcattaagaaggatgg
aaaagtatgaagctctcaaagcttggcaatctgtagaccacgatcacttacaagtatagatgttaaaatattaaactgaattcagct
atgtatgaaaatagtaggccgggcgcggtggctcacgcttgtaatcccagcactttgggaggccgaggcgggtggatcacgag
gtcaggagatcgagaccatcctggctaacacggtgaaaccccgtctctactaaaaataaaaaaaaaattagccgggcgtggtg
gcgggcgcctgtagtcccagctactcggagaggctgaggcaggagaatggcgtgaacccgggaggcggagcttgcagtgaa
ccgagactgcgccactgtactccagcctgggtgacagagcgagactccgtctcaaaaaaaaaaaaaagaaaaaagaaaat
agtaatagatgatgagcaagtaggattgttccaggaatcccagcatgtcacaaaatgagaagaccttctagtatagtttacatatta
acagatgagtggctttctcagatgccgagaaagcagcaacattctctcagatgttgatgatgaaacttctttagtataacactaatta
atgttggaaaaacaggctctccattttgaaaaataataagatatcacatatttgacaaccataattccatataaatccagtatttaaat
gtaaaacataaaactagaaaagtagaaaaatataaaggtaattttccttttaaaataatcttggagctggaaagaagtgtcttaag
aactgaagctgtaaatcatagggaaagattggtaggtctaatgggggcatggcctgaataaagttaaaagacaaatacagaaa
tggcagtaactgctaatacataaaaataaaggttaacatcctcactatagaaagagcttttataaatcaatacaaaaagacagtc
actgacctagagaaatagacaaaggacatgaaaatatggttccaaaaagaaaaatcgatggctagtaaatatattttttcaatgt
agcctcattaacaagtttttattttttgcctctcaaattgataaagtttaaaaaaagtaacaatgagacagaggctgtgggcagtagg
aatactgtttttgagtgtaagtcgatagaacccagtaatatggactgagttttaaatgaaaatgaccttctactcctagaaacgtgtttt
accaagatgtctatacatggatgttcgttgtagggcatgtttgttttttaaaattaaaaaaaaacttggaaacagttatgggaaattgtg
aattgacatgtccatactacttattgctgtctgtttaagtggtctgtgttgacacagaaacctgtctacacagtaaagaaaggagttgct
ggggccagacatggtggctcacgcttgtaatcccagcactttgggaggccgaggggggcagatcacgaagtcgggatttcgag
accagcctggtcaacatggtgaaaccctgtctctactaaaaatccaaaaatcagctaggtgtgatggcgggcacctgtaatccca
gctaatcaggaggctgaggcaggagaatcgcttgaaactggaaggtggaagttgcagtgagcctagatcgcgccactgcactc
cagcctgggcgaaagagcgaaactccgtctcaaagtgaaaatagcaggttatgaaacgagctgtgcccccattttacacacgtg
tgccattgtacacttctgcctgggaatccactgattatgttcagatgattttttttccatattggaattgcaattgatctatttgctcatatgtttt
caaaatctcccacagaaaatgtatattacttttgaagttagaagttagcaataagagttgttagctaaaaaacagaaacctatttgct
atggaagatggcgggttcactcaggggtgcccgatgccatgttagccatgcatctgtccccgcatggtcccgtcctccaccgccta
ggagatagtggaccatcagtgcctgaatgcaaatcatagtgagtggtgtgcaggaaagaggttgggcagggcctgtctgagga
ggcatcgtgggggccgatccttgaagaatgtgaaagggacaggagaagagcaggcagctctcaggcagaggtgaggggca
gtgcaaacgtggagcagtggccccgatcactcagggacgtggcagccttgggaaggaactgggtttattctgaatgcagcgtga
gacctactcactgaagctgtaggacatgctttccatgtgctgtgacgtgatctgcaaggaagattctaggcagaagcaacaattttg
tgattgaaaaattccacataaagaagcaattcctgattccctgtactgacctgaggtacctggagaaacttagttaatcttttcagcct
cggttttcccatctgtaaaatgggaagcctctcagtgtccatcctgtggagctgtaaaggctgagtaagggaggcctgtgggctgtg
tgccataggccactcttagagtgagtagctgtggttttggctttgtgtttggtttgcatagatactagctttaaaatgtctacttgacaggc
cggacaaggtggatcacctgaggtcaggagttcgagaccagcctggccaacatggtgaaacccatctctactaaaaatacaaa
aaattagctgggcatggtggcgagcacctgtaatcccagctactcaagaggctgaggcaggagaatcgcttgaacctgggagg
cagaagttgcagtgagctgagatcgcgcctttgcacttcagcctgggcgacagagcgagactccgtctccaaaaaaaaaacaa
aaaaaaagtctacttgaggctgggcacggtggctcatgcttgtcatcccagcacttcaggaggctcaggcaggaggattgcttga
ggccaggaagtcaaggctgcaataagctatgattgcaccactgcactgcagcctgggcaacacagtgaaaccctttctcaaaa
aaaataaaataaaatgtaaaatgaaataagcattgctagaaggtgttctggaagctttcatcttaatactcttatttgttgattgcgtatt
tttctaatttggggagatggtttggaaataattgttattaaatcattttgtgatatattttagtccagccccttgtttgttttgttttgttttttgttttttt
tgagatagaatctcactctgtcacccaggctggagtagtgcagtggcgtgatctcagcccactgtaacctctgcctctgcctcccgg
gttcaagtgattcccctgtctcagcctcccaagtagctgcgattacaggcgcccgccaccacacccagctaatttgtgtatttttagta
gagacagggtttcaccgtgttggccctgctggtcttgaactcctgatcttgtgatctacctgccttggcctcccaaagtgctgggatta
caggcgtaagccactgcacccggccaattttttttttcttatggaaaatctcaaacatgtacgaaaacagaatatcacataatgacc
actcacgccccacacacgtgctcatcatctggcttcagctgccggccctgctgctttttattttgatgttataaaatactgctgtcccggt
atcagtatctttgtgtgtgccctgtcccctgaggcagttgttgatgaaaagtggttatatttcagagggtctgggctatcacatgcttaac
tgtctacttcacatcacacgcgggtttggagccataaaatgctaaagcggaaggacctccgccaggggccagccaggtagccc
accctgtgctacagaggtggcatcacaaacataagttgcagcccttccagaagcggccttgtttacccagaacccacttccctctc
agtcacctggtttgcggtgcacttagcatccccttcattgtgggtgccttgaatattcttcataaataacaactgatggttttttaaaatac
tgtatcttattgcaaccagttagctcttgtcaagagccatttatcacagcatctgaaagagaaagggactctgtgttcattgagtggtg
gggcgggaaagatgattttttctttagggcctccatatttcccttaaatttaagccttctggatattctaagaggagggattgcttctaaa
cttctgtcacgctgggtttgacattttcttacaggtgtggaaaatggtctaacataatgcctgtcacaaagtaggtaaaaatgtttgctg
aataaaggcatggattctgtaatttttgctttgtaagaaaaggctattttttatcatggggaattttttaaagagacctgtttatagtggagt
cacatcatatgcctcctgaagcaaatttagatatatgctgagccatgaatttttttttttttttttaaagaaaaatgaggccgggcacagt
ggctcacgtctataatcccagcactttgggaggccgaggcaggcggatcacgaggtcaggagatcaagaccatcctggctaac
atggtgaaaccccatctctactaaaaatagaaaaatttagccaggcatggcagcgggcgcctgtactcccagctactcaggagg
ctgaggcaggagaatcgcttgaacctaggaggcagaggttgcagtgagccgagatcgtgccactgcactccagcctgggcga
cagagcgagactccatctcaaaaagaaaagaaaagaaaaatgacaagaattggccatttaaaattgcaggtgactgccctgg
catcagcgagtgtgcccttgccatgaagtccccagtcagtgcggttctcacagcatggttcaggggctcaccccagccccacgcc
atgcagtgcacatctgcacaggtctgctctgacggcacggcgtcccccaccgtagaccctgcatatgatgtggctccatgctagtc
atccccttcccagcagccgatgctcaggtgggtagcagggcctgcaaagatttccactctgtaacatgtatcataattctcacctttc
ctcaatagCTTCCTTTGCTCCAATAAGCTTTGCAATCAAGGCCAAAGAAAATGATCTGCTTC
CCCTGGAAAAAAATCGTGTTAAGCTAGATGATGACAGTGATGATGATGAAGAAAGCAAA
GAAGGCCAAGAAAGTTCTAGTAGTGCTGCAAACACTAACCCAGCAGTTGCCCCACCCT
GTGTAGTTGTTGAGGAGAAGAAGCCTCAACTTACCCAGGAGGAGCTAGAAGCAAAGCA
AGgtttgttgatagcttttaaacttcttgaaagaaaggaaatacacaaatataagatttatctgctaagccaaaaaatctcgaggct
gccaactagaatctgaagcctttggaaatcgacctatttgggagttgtgtaacatgtctgaggttttgaaacgttctcttttagaggaat
gagctctgctcttcactgagcctcaaatgcagtgccgctggcagtttgttttcgaagaaactgagttggccgtcttagctctaatgcgc
cacagtggaatgcattaatggcagctcactttgcacttggctggcagccccagggtaaaaggctcagcctgtcttcccagctcag
gaaccaaactaggagatgccctcttgtgaggctgcctacccacagaaccattgggcccttgaaggtggtgtgtccccagctggttt
tccggctgcggctcatcttcatgggccgcagtgtggccaccacacccaccccaacactgctggcagcatggggacagcatgta
gtcttcccatcccgactccagaataaattctgctctgcattaaagcagtcaaataatggttgctgcattgtggttgttatctattctaactg
attttcttaaattgcttttcctgtatacacacattcagatcaagcaacatttgaaagaggccaattttcaggccaggcgcggtagctca
tgcctgtaatcccagcactttgggaggctaaggtgggtggatcacctgaggtcagaagttagaaaccagcctggccaacatggt
gagaccccatctctactgaaaaaacaaaattagccgggcgtggtggcacacgcctgtaatcccagctacttgggaggctgagg
caggagaatcgcttgaacccgggaggcagaggttgcagtgagctgagatcacgccattgcactacagcctgggcaacaaga
gcaaaaactccgtctcaaagaaaaaaaaaaaaagccatttttcaaccacaatccaccatcaagaacttccattgtgctgtggtgtt
ctccctaagcaaacttgtactcatgcctgtacatctgaatctgtccttcctgtgtgtaaactaaccaactgtcggatcatttggaataaa
acacttatagagtattcattgcctggtgtgaatattttggatatatgctgagagccactctgaggttttcattattccagctttcgttagtgt
agagtctcaccaaccttctaactctgaaagtaaaatgtccaaaaaagggcacgttataaactaattctctcaaaatttgatttgtcca
atgtatgtacctattcagaaactttaactaactgcattgtatgacacttttgcaacctgtgaaaattaagatcagataaaatactgtttg
ctctaaacttctcttttttctttgtttattccttaagCAAAGCAAAAGCTGGAAGATCGCCTCGCAGCTGCTGC
CCGGGAAAAGCTGGCCCAGGCGTCTAAGGAGTCAAAAGAGAAACAGCTTCAAGCAGAA
CGTAAAAGGAAAGCGGCGTTATTTTTACAGACCCTCAAAAATCCTCTGCCGGAAGCAGA
AGCTGGGAAAATTGAGGAGAGTCCTTTCAGTGTCGAGgtatagtaaaatcccacattggtatctgcggg
gctgtgtgatacatagaggcagggaggatgtgtctccctccagctgccctagtctctggcctgagtgagggatatgagctcccagc
tcttcctcccgacatggttgagtggcttttactctatagcagtgaatctaagagtttgccagcagtctcccccgtcagtgcacagtcac
gccagcagcaaacactgcccgcgatttcaggggagcctctgcttcacggctgcccttatggggctggcaggagggcttgggga
gtgcctcccatgggtcctgctggggaaatgtggtggacacacttcactgaagccccgcctccgcagcagcaccagtattgcgctc
acacgtggggcagaaatccttttgccacggtctgtatcaatgtcagcactttaattaaagagaaaaaggaagagggagttaaga
gaacagactccaggagtacatggctccttcctcagtggtgtgagcaggaatagggccttacatgggggtcatcacgtggctgcctt
acaagtctccctgccaaggagggggtgctcagaacagtgcctcagaccagaggccttcagtagacactggctcctgagtgcca
aggggattgctcccttgtgtgtccgagaccagaggccttcagtagacactggctcctgtgccaaggggattgctcccatgcgtgtc
cgagaccagaggccttcagtagacactggctcctgtgccaaggggattgctcccatgcgtgtccgagaccagaggccttcagta
gacactggctcctgtgccaaggggattgctcccatgcgtgtccgagaccagaggccttcagtagacactggctcctgtgccaagg
ggattgctcccatgcgtgtccgagaccagaggccttcaggaaacacatgccttccgcagcagcagcacagcaattaatcataat
cagcaaaaactctacttttttttttgtcacatcaatttagaatcttttaagtttaattttagattctttatagtagttatgtctctgaattttattttgt
atttaaactacaagaatatgcagaaattctttggggagtttaggagcattttggagacataactcttaaagtaagaaaaataataga
gtaggacacatcctttgaggattaaaggagggttgtctttgtatcaataaactgtgacaaaactgggcattttagtagctagtcctgta
attgtaggtgaattaaaagctgacaacatttgaactataatattagaatgggtttacatctacaattagacaatagctaaaaagttgt
ggttttatgttatttcaagaacacttaaaaatcattttataaaatctttctcaacctaatctctctctttaaaaaaatgaatgaacacagg
aacagaaaatcagacaccacatgttctcgcttataagtgggagctaaacattgagcacacatggacacagagaagagaacag
cagactcgagggcttcattgagggtggagggagggagggaggagggtgaagatcaaaaacctccctgttagctactatgctca
ctgcctgggtgatgaaagaaataaaagttggaaagaataaaaaaggtagtaactccgggaattttactttttgaaaagtttcaaac
cttcaaaaaattggaaagaatgggaagatgcccccagcaccccaggcgattgcatgcgcgtgctcgctcatctatatgtgcgca
cgttgacccacgcgtgctcgccctctctgagagtcgttgcatatgtggtgactgctctgccctgaatactgcagctgcatttcccatga
agggccttctcctgggaaacacagcactgcatgcagattgtccaccgatggtgtccatcacttcctctgcaggccgcagccacgtt
gctccaagtggccccacgtgtcttttgtagatctttttttcccaaagtacagaatgagcctttcactttaattatattgacgtttctaagagt
ccagggccattattgaaaactgattttctgcttgaagtcacttcgcttatttttctgtggaaaacaacattctaagctcagacttttcaaat
gatgctgaaggctgaatcagctttcttgttttgggagtcagtctgaaatcctctcacatctggcaggaggcctcagaaataataactg
acgggcaaggaggggagaattagaagagcagagaagatgagtttgtgtgagaccctgtcgagtccccgagtgccgcagggt
gggctcctgccctgagtcccgagtgctctggccacccgctgtagcctcagctcctctgagccatttgacatgccagccccagaaa
cgaacattttcaggcaaggtgggaacccccagcagccccccgggacgccgtctcacagcctttccacagctcttcagagtcggg
gctgcctcctggctcctcacttcagccagttatggccgaaggatctgtggtcattccttagctttaataggatttcttggctggacgtggt
ggctcatacctgtaatccgaacactttgggaggccaaggcgggtggatcgcttgaggccaggagttcgagatcagtctggggtc
aacatggtgaaacctcgtctctactaaaaatacaaaaaattagccgagcgtggtggagcatgcctgtaatcccagctactcggg
aggctgaggcaggagaatctcttgaacctggcaggcagaggttgcagtgagccaagactgcaccactgtactccagcctgggc
gacagagcgagactccttctcaaaaaaaaaaaggatgttctgcagcaataaggggatgaaatacacaacaacaaaaatgat
catgaggacgcttgtagccacacagaaaatgcttctgatgtaataagcaggagaagcacagtataaaatatatccacttctgtggt
tacagccatgaaaatatgcatgtagcaaggagggaagggaatttaagaaagtaagggacctgttacagtggcgtacgggttctc
atgttttgatatcgtttgtgcagcgggtaaaggggttaattgaaagacattcacaggaatgctttaaccagttacattacatgactata
cgtgtatgtcgtcataaaatttccagtgaaactcagtcacaagtataatttatcactagcccagtttttcccaatctgctgtagttccgca
tcacagcaaccagaattatttccttataaacataagatatgttacagcttaggtctgtgtcctatttatttattttattttatttatttatttatttgt
ttgttttttgagacagagtcttgctctctcgcccaggctggagtgcagtggcgcagtcttggctcactgcaacctccgcctcccgggtt
cacaccattctcctgcctcagcctcccgagtagctgggactacaggcacccgccaccacgcccggctaatttttttgtatttttagtag
agacggggtttcaccatgttagccaagatggtctcaatctgtcctatttatttttacacgtaccctctcacctctcctgtttgcaggcattg
gtttttgaatctgtagaacatagaaatgagcgtttaaatcactaggatgctctccctggatatatgtgtgtgtctgtgtatgcagattaca
gctaccaagccatttcaacaaaaatgtaatggttgtagcagatgatgataaatgtctttaattgcttctgaaacaaaaatacttgtaat
taaattggcaattgccataaagaaaattcaaactcgaaaatatttttagcctaaaacaacttctgggacaggttacccttgactttact
aagtattctagcatctgctttactcgctgatgttgagacatttgacccagctatgtagttgtgaaattctcggagtccaggaggacttga
gacaagaccacattcggccaccgcacgccctgggtgaggaagcctgcgtggctgagggcacgtcggcaccaggaggctcat
ggcacccccaggtctgtcggggccgtggctagctcgggctggctctgcagggtggcatgaggacactcccttacacaaggcctg
gcataacatggcaggaattttgctgtcaccttaaagttaactgaaaacagccacagtgcagcttatgtgcctgaaggacagtcact
tctctgtctttactttctataaaactgatgtatacatatgatttttaaagttccaatgctagagaaaggtataaaacaaagaggagaggt
cctttctttcttgtgtatttttttaattcctgtggaaatggcactttttaaaattcctccaattctctcccttctgtagagtttgagtttttaacataa
aggttaccattttaccgtttttaactgtgcagtttatgctggcattaagtatattcacactgtgcaactattaccacccaaccgttcccag
gatgtccatcttctaaaaccaaaactctgtagccattaaatagtaactctctgccctcccctcccccagccctggcacccacctgctt
gcagcctctgtgaacgggactcctggggaccgcatgtgtgtgggattctgcagtgtctgtgcttctgtgcctggctgacttcacctag
cgtggtatcctctgggtccagccatgcagcagccgcattggacccccttcctttttacagttgaatggtgctcggtcgtgtgcatctac
cccgtttgtttccgaacactttgggaggccaagtgaatccttgaggggaaaaactcaattcgattcatgtatgtggaggtgtaaaac
ctcataggggatcataccctacatatttttctgacaacttttttcacttacagtagagatccttctgttatcaatgcttacagtagtgtttggt
gtgttacaggcactcggtaggtatttagtgaccgaatgactttaagtagtttacctgtaaatcaccccctctgtgtgtgcccctcccctg
ctgtgggaaatgtgagctgtgggtctgtctttgtgaacgatgccgctttcacccaatctgtgtcctgtgtcttgtgtactccagtgagtgc
acccacgggggaaatcctcaccatggaattccttttttaatttgagatactgctcagttgacctctgaagggaccgtagcagttttattt
gcccccgtagggtgtgcgaactatttcttcatgtccctgtcggcattggttaccatcagcctttcggtgggaaagcagcatctgcttta
acttacatatttaatggtaagtaacactgagtcattttgctaatgtctcttttttttttttttttttttttgagacggagtctcgctctgtctccaggc
tggagtgcagtagcgcgatctcggctcaattggctcactgcaacctctgcctcccgggttcaagtgattctcctgcctcagcctccca
agtagctgggactataggcgggcgccaccacgcccaggtaatttttgtatttttagtagagacagggtttcaccatgttggccagga
tggtctcaatctcttgacctcgtgatcagcccgcctcagcctctcaaagtgctgggattacaagcctgagccaccgctcccggcca
ctttgctaatgtttcttggcattgtctgctgccttttgaactggctttcccttagcctgggcccatgtttcttcgacctaggaagggccctttct
ttgtcttatgctgcgtcatgttagctgtttgtcatcagtgtcacaaacattttttccccggtacatttctgacctgccatacttggaatttatttg
agtttgaggagcacgtcactatcgatgcaacgcattcactaagtcatacatcttttccccagtgactcacagagccttctttacaacg
tgctgaattcccatttgtactcgatctttctgcatccatgttcctgttcagtcccgttgatcgttaggaaaggtgattttacatattcaggtac
agttgtaaatgatgtcctaaagtgtgcctattggcatggaaagatattggcagcactctaaatttttcaagtggcatataaaatatata
aaagcatatgcaaaaatcatgcatataaacctgcataggagactggagagttcccttgacccttctcaggactggcacaggggg
tggctcgttttctcggctgccactcaatcccttacgggagggaccacacgaacggacaggtgcgggaaccagagcaaaggaa
ctctcctctctggcgggagcaggctctgcgctggcctcacggcagcctccaagcatattacaatgctcttttagctctgccatctggg
agtgggtgtctgtgacccctggagcctcagaaagcctgtgttacaatcagtgttgagtgttaatcagctcagtggagggtcagggtg
acagcctttacaccctgccctcttggtacctgagttcttgtccggcgtccagcaagaatcaggtcacacgaacgaattaaagggtg
gtgaatatggaggactttattgagctgtggaagtggctctcagcagaaagggaagctgacaagggggtacagcaggaagata
attttcccctggagtctggccatccctcagccaaactcctctccaacatccagctgcttcctctcctctctttgctcagatgctttctcttct
gtgtgtgtcccctttgtctggagtctggggttcttatgggcacaggatagggggcagagcaggccaaaaggcaacattcaggtgg
gaaaacagggatagttgtcactttgggccacgggtccaggcttgagtgtgaagccctcaccagtatttccctgcctcctgcctgtat
cacatacattctctcttcattcctagtgtgtgttcgtaatccaggatgaagagaagggagaagtcttcattgaaccccttttttcatgcg
gtcatttacatagtagcaacagactgcaggatgatttcttagattccacaaattttttttctttttctttttgagagagtctcactctgtcgcc
cgagctagaatgcagtggtaccatcacagctcactgcagccttgacctcccggcctgaagcactcctcctacctcagcctccaaa
gtagctaggacttacatgcacttgccaccatgcccagctaaatttttttgtatttttggtagagatgggattttgctatattgcccagactg
gtcttgaactcctggcctcaagcagttctcccgccttggcctctcaagatgctaggatgacaggcatgagccactgcacccagccc
acaaatgttttcaagttactgatctgccaagtttacaattccagtaagagtttgaaaaggaaataggaactgaaacctgcctgtgttt
gctgaatctctgctgtgtgctaggtgctgaggtgctttgggatatgcattagaagcttgcttgttaaccagtgaccatgactgcatttga
gctgttgctgttcacacatgggcatttccatcaggacagcacagccaggaggagagtggcggctccgggacctggggctcagg
cgaggccttgaggagcttaccagaatagtgagggcccacgagggccaaagacccacaagtggtaaaggacaggtggcccc
actcaggaagacactttctcaggcagaaccggaatgacaatgggaggccagttgtggagagcctgggacgccagaataagtg
agcacgagagaccgacaggatgagagccgcatttccgctgagacagtgtggctgcggggcacggggcgctggagcagagt
ggaggcaggggtgggaggatgcacctgggcaggacgtggtagggcagtggggctgggtgaagggatggagagcaacgcc
gcagtgttggttatccctttcatagttaatgtagtgtccttcacaaataagatttcttttattttcaaatacaatcagatacaaagtcagtct
gcttttgagcggtttgttttgccacagtaggaaataatcgttgctggttcatgtgctaattttgttgccaaatacttcatcgtgacacaggg
ggactaatcaatgttaatttccagtgttacagaagtggccggcggtaagctgttaatgctctcataaatgaccatttttcagaagttatt
tgctttgtcccggactctacctaaaccaatgtacgtctgcccccctacattcaaacatgacttccgttttgatcatttttgctggaatatta
aaaatgcatctcaaaggcagctgtggtttctgggaagctgtgtttggcatcagtccttgttcacttttagcacttgaagctgaaaaaag
cagtaatgtcaacataatgaaccatcttaattcagcctggcagaggtcacaacagctctagttttcaccttcatggtgaaggatgat
cgtgttgttggaataaatagacctggacttgattacaagtgacatttgaaagtgttgattcagattgtcccgtcgcttcaaaatggagc
cctagtctttaagcacagtggtgagataagtattattaatgacaggcattagttaggataaaggcaaaaaaaaaagtttggaggct
caaatcattaagttggcagtagaaatatgaatagaaactcagctggagagttgactcctcgcactcctgtttgtcttgactgtgcctc
agatggcgtctcgcgcccgtttggttttgtctttcacagacgtttgccagggaccatgtttttccatctcccctctgttttaacacagcgcc
ttaccaatcacacaccaaattagtgcagtgattttgtgagcgtggagagagtaaatgaggagagttcttcaccagaaaaagaca
gcaaagacgtgtttctcttccttctcgtcacagaacaaactccttactcgagggtggagtatgtgtctcagctctccttctcttcagctct
ctctttgttttcctggggaaatcccgggccttgttgaaaggacctgcagcagctctgacttcccgaacactcacaggtgcccgtgttg
aggttcccaatggcgtctttcagcccctgggccggcttgctttctgcgcagcgtgtgctcctgatgtagaggccgtggatactggcat
ttttttagtgcatcagctgatttctctggtgtccacccagggctcgcctcagaggatgtgctcagctcgcaaacctgtgttctttgctcttt
gcagaatggaagccctctccctttcggtgtgtatgggagaggccatagctaggatgttgagcctctgaagttgtaaagcttactacc
tttttatttattgtatgtttaatttaaaggatcatttagcattgcttgtgggcaaatcctgactaatgccagagtgggggtgttcttggatata
gagcttgctttgtcattggacgtttgtgtgttagaattatgtaagcaataaaatattttagctgggcacggtggttcacgcccgtaatctc
agcactttgggatgctgaggtgtgcagttcacttgaggccaggagtttgagaccagcctggccaaaatagcaaaaccctttctcta
ctaaaaatacaaaaaaaaaaaaaaaaattagctgagcatgatggcacatgcctgtaatcccagctactcaggaggctgaggc
acaagaatcacttgaggccggaaggcggaggttgcggtgagctgagatcacgccactgcactccaccccggcaacagagca
agactctgcctcaaaacaaacaaaaataataaaatatttaaaagtttgacctgaaaaatattgttacacttaacagaattttaaatg
agaaagacctttttgataagaactgtcccacagtaaagtggatttttttgccaaaatgtccctggagataatttaggcagagacttaa
agatgaacctcatagcggccatcagatcccaaggaggaattcatccctgccctcttgcccgccgcacacccacaaccagggag
gggcattagagagcacagtgtaaacggaaacagcaaggaggctgaacagagggctgagaaatcaccgtgccatcataaag
cagccagctcaagtggaaactcatcttaaattggggcctgccccaccagggctctgctgaattgcttttgatctcaaagccaaagc
aagaagcataactgtagaagaatcgtttctacagtgttttcccgcagccagttggccttgccacagcggacctaaggagaggaa
agaagggagggaagcccccttaccactttgcctttcacagatgccgtcctgcgcacactgccgcgggctgggctggagctctcc
ccggggagcagctgggggcagcctgggagactgggtcccaccccagcacctaacctgaatttcttcgaggcacaaaggataa
attgcagatttttcactgtgtctaaaggtgtgaaatgtttaacagctataatttaaaattcacttgaagtgaggagagagtgagctttct
gggtaaagaggggcaggctgcaggcctatgctgttgaagggtgctgtctcctgatctggttccgatgcgctgtggtggaaatgtgtc
agcatgcattgaagattcatatgctcttctgtatgtatgtaacactcagatggagaggttttaaaacatcaaaggggagcctagacc
ttctttaaaaattattgtcagagtagtgccgatactcatttaaaaacctaacatcggaggtttgaggaatctctcctctggtagttaaaa
ctgttttttttgtttttccttaagaactatttttttttattatactttaagttttagggtacatgtgcacaacgtgcaggttagttacatatgtataca
tgtgccatgttggtgtgctgcacccattaactcatcatttaacgttaggtatatctcctaatgctatccctccccgctcaccccacccca
caacaggccccggtgtgtgatgttccccttcctgtgaccatgtgttctcgttgttcagttcccacctatgagtgagaacatgcggtgttt
ggttttttgtccttggtgatagtttgctgagaatgatggtttccagcttcatccatgtccctacaaaggacatgaactcatcattttttatgg
ctgcatagtattccatggtgtatatgtgccgcattttcttaatccagtctatcattgttggacgtttgcgttggttcgaaatctttgctattgtg
aagagtgccacaataaacatacgtgtgcatgtgtctttatagcagcatgatttacaatcctttgggtatatacccagtaatgggatgg
ctgggtcaaatggtatttctggttctagatccctgaggaatggccacactgacttccacaatgggtgaactaaaaaggaacgtatttt
ttcccagcgtagcatctctaatactctaatactgtgctcctcttgttggctccggctgtccacagcctgggggctgggaagagagtgc
tgcctgtggaaatgctcgggaaccagagggttcactttctccttttgcatcctgggaggtgacaaggaggtcactctggatagccac
aggaggagactttctaagagatggttgctgtgtttgttggtgtgaggggcccaaagttgaaattttatagatatacatcttcaatgttct
gttttccctgttaacacccagattttccttttattcttagGAATCCAGCACTACGCCCTGCCCTCTACTGACTGG
AGGCAGGCCTCTGCCTACTTTAGAAGTTAAACCACCCGATAGGCCTTCGAGCAAAAGC
AAAGATCCACCGAGAGAAGAAGAGAAAGAAAAGAAAAAGAAAAAGCACAAAAAAAGATC
TCGAACAAGATCACGTTCTCCCAAGTACCATTCGTCATCCAAGTCCAGGTCTAGATCAC
ACTCAAAAGCAAAGCATTCTCTTCCCAGTGCCTATCGGACAGTGCGGCGGTCGAGgtggg
tgtgaagggggcagcacctctggtaccctcatgacccccatgtccttcacaggacacccagtagagctaggtagaacgtttaaa
atcagtgccgctttcattaagcagacgcgtgtatgcatgtgcatgtgtgccctgcaagtccaagtaagatctttttcagatttttgtttgttt
tatacttaactttttcttttttgagacagagttttgttcttgttgcccaggctagagtgcagtggtgcgatcttggctcactgcaacctccgct
tcccaggttcaagtgattctcctgcctcagcctcctgagtagctgggattacaggtgcccaccaccacgcctggctaatttttgtattttt
agtagagacggggtttcaccgtgtcggccaggctggtcttaaactcctgacctcaggtaatccacccaccttggcctcccagagtg
ctgggattacaggcctaagccaccgcgcaggcctatacttaacttttcaaagttcataaactactgccaggtttttaaaaattggtttg
tttaaattctaatggttcctggaagcaagcctaccacatttgccgattgtgtgaaagattcacagggtggtgtgctgggggtcttttgttt
tatttgtataagtgaagtttcccatgctaatttgtctcaaatgtgtaaagttgcaagacaggagaactctttagcactggttctgggtttg
gattctctgctctgcacacgcactcaccggcaccgcactctgcacatacactcaccggtgccacactctgcacacacttcgtgtgg
caccggtgagcgtgtgtgcagagatgcagcgacggtgagtgtgtgtgaagagggcagcgcggatgagtgtgtgtgaagaggg
cggcgcgggtgagcgtgtgtgaagagggcggcgcgggtgagtgtgtgtgaagagggcggcgcgggtgggtgtgtgtgaaga
gggcggcgctggtgcagaatgtttcctctccaccctccctccaggagtcactattaaaccaaaggccttcttgatgaggagccagtt
tttcagaaagcaggttaacatttctggcagcagaaattaaaaatgtaaaaacatttaagagtcacagaatttacatcttggtgaaaa
ccactttttaaaaacaaaacagtggctgacctacaggaggttggcacagcttgccctgttttcagaaccccgttacaccttgggttc
gctgctgaacactggctgactctcctcggtttctctaacgccgcactgactgtgctcatctagtttttcttctggaattggtgttagctctta
tgtttctgtgggaaaaatacacatgccttgggagctttacgggctttttaagtgtaattttacacatttgcctctctgaatatatcctaaaa
acaatatgcttgctttctttacttatttatttatttattcatttatttatttagagacggagtttttgctcttgtttcccaggtgggagtgcaatggc
acgatcttggctcactgcaacctctgcctcccaggttcaagtgattcttctgcctcagcctcccaagtagctgggattacaggcatgt
gccaccacgcccagctaattttgtttttttagtagagattgggtttcaccatgttggccaggctggtctcgaactcctgacctcaggtga
cccacccacctcagcctcccacagtgctggggttacaggcgtgagccactgtgcccagcctgctttccttatttttaccctggccaa
cacttaaagtttgacaagcatttacactcctctgcagtgaaattggatttgactccatgataaatcaatttgatctttcactctacatttttg
cgagtgttttaaacgtttcatcacttcatacccttatacacgcaaaaaagaaaccttgctattttctaatcaaatgaacagttttgctaat
atatcttcaatttttgaaggctcccaggaacttgtattgtatatcgaagctttttaaaaatttctcatttgaggccaggcacaatggctca
cacctgaaattccagtgctttgggaagccaagatgagaggatcactttgaggcctggagttcaagactagctttggcaacatagtg
aaaacctatctctacaaaatatttttttttaattagccaggcatggcagtggatgctttgaactcctgagctcaagcgtagagtctgag
gtggaaggattgcttgagctgagctcaggagtttgaggctgcagtgagctatgatcacgccactgcactccagcctgggtgacag
agcgagaccttgcctctaaatgcaattaaatgattaaaataaaaaatttcccacttgaatatgtttcttacgacattacatagctgaag
ataggcataaacaagccctcctagtaaccacattcagtaaaattcttcccaattttccttttctacaggctcaaaaggaagcataatt
ccttcctaaatcccaaaccttgggggaccgatcattgtaagagctgttcatggtgtttctttagcgtaagaaattagctcagctttcatg
tggggagtttttgcaaacacagcggatgtgatgtctgatatttccgggtatcctaccattcacctctaaagacaggtgatgccgtggc
ccccagcttttcccacattggcatattcagagctgaaaggcttcacctaacacttggaatttcaggtttctaagttgtacatcctttttgtt
gactggtctatagtagaaaaggtcattttacatattatttgaatgatttattttagaatcgatttagagttacatatttttgaataatttagaat
agctttagttacatattacttcacatatgcaaatatatcttattatttttttttttttttttttttttgagacagagtctcgctgtcgcctaggctgga
gtgcagtggcgcgatctctgctcactgcaagctctgcctcccgggttcacaccattctcctgtctcagcctcccgagtagctgggact
acaggcgcccgccacctcgcccggctaattttttgtatttttagtagagacggggtttcaccgtgttagccaggatggtctcgatctcc
tgacctcatgatccacccgcctcggcctcccaaagtgctgggattacaggcgtgagccaccgcgcccggcctatattatttttatat
attcactgctgacaagtccaagaagcaaaatcctactcatttgtttgtaactttcagttaaaagaaaaaattaaggtaaaagttacct
gagtgtggtttccaccgtgatggtaggctaccaattttaatccgacctacgtttaaaacactttacagcgtcagcagagcaaagtgtt
tccagaacactccaatttttaattagtctccatggccaaggaggtagtatctacatacttctagttaattttagttaaataagggatttaa
aagcatttgattttgcaactgagacaaaatatgaaggcaaagtgcaagcttattataaaatgaaaataatattataaaacaaaac
cttccaggtgttggattgtctagcaagttctaccgtgggtgctggcccctggcattggttcccctccacagggccaagggcatagct
gggtgcagagaccggcagtgccgtggtctctggagtctgaggacataagttaaacaagctagtcaagccccagatgcttggga
ggcagaggcaggaggattccttgagcccaggagatcgaatctagcctgatcaacatattctctatgacaaaagaacaagaaga
agaagaagctggtggtttctcaccataaccttttcttgtggaattctgcctcagctcttctgggaacagtgagtgcgtgttttatttagtag
gattgcatttttctaaactggctgcaaacctgcctcctccatccaagctctgccagcaataatcatttccagggatccaagtggcttta
aaatgcaagttagaaatgggaggggtggtgatctcctcagtaatatgaattattggagtataaaagataactaaattttaaccaaa
atattgaaagtgttaatgctgttgttatcagatagaataaactgttacaaacgcagcctccactcagaatggatcggacttgtcacttg
ggcctgaacagacctaattgatcatttttcatgactgctgccagcccacagtagaataccgcagttgttaatatttctaattgggtagg
atgctacatggaatgtattttgttttatatattaaattactaaaattctatataaaatacagaaagttaagattagaaagccttcttacag
cacaacgaatatttatttaatggctatactgttcctgtggttgaagtcccatgtatttagtatgtctaagttatgggcgactctggatctcc
aaaggcaaattagtcatggaagaatctttagttttggaaaatcactatgttgcttctcaaaaagtatactagttacgacaaggtagtat
ttagtgtcttttacatcaacattgaggctggcacggtggctcacgcctataatcccggctcttaaggaggctgatgcaggtggatcac
ctgaggtcaggagtttaaggccagcctggccaacatggtaaaaccccatctctactaaaaatacaaaaatcagccaggcgtgtt
ggtgtgcgcctataatcccagctactcgggaggctgaggcaggagaattgcttgaacccgggaggtggagattgcagtgagcc
aagatcgtgtcactgctctccagcctgggcaatagagcaagactccgtctaaaaaaaaaaaaaaaaaaagattaaagtaaaa
tacttttattgtctgttttcatttgtattttgatattgtatctggttctctatgttaatggaatgaagaagtactcatgtagttcatttacaacctga
aattaaattttaataagtatcagcttgaaactaagtttatttttaaaacttttgctaagatagtctcttgtgttcatttagttatctaaatgcatc
ttcagagttagcctgggcttctgggagttctagatagatctttgaatgttgtcattttaagatatcttccagtatagagagctatatgataa
aaatatatttctggccgggcgtggtggcccacgcctataatcccagcactttgggaggctgaggcagacggatcatgaggttgag
accatcctggctaacacggtgaaaccccgtctctactaaaaatacaaaaaattagccgggcgtggtggtgcctatagtcccagct
gctcaggaagctgaggcaggagaatggcgtgaacccgggaggcggtgcttgcagtgagccaagatcgcgccactgcactcc
atcctgggcaacagagtgagactccgtctcaaaaaaaaacattttatatatatatatatatatatatatatataattctttgtagaaatta
gctccctaaatacttggggttggtgaaggagactggggatttggaagacttttcttaggagtcttgtttagcattcagaagggactca
ggccacactgggtttctattttaggttgaaagttgtggctcctcactgccctttttacccacaataaattgcatagcaaatccgtaaaag
cgatgactcatctcctaatcctgccccttaaagggggaaaccagatgcttgcagttccccaagtggtagtgttgatcatgccaaggt
gaggaccgtcgttccatcccttgcaaagtgaatcaaagtgaattgtagccaaacacagataagaccagagggtgtctgcactga
gcagtccaggaaggaggggagctgcagtggctgtcaccgggctgggacacgaggaggaattgcaggtgaaatcagatccag
tttcaacttgaggaaaattcagccccgggagctgctggtagagcccagaccttgatgctgagtcatctgcacagagaattccgtga
cagaaaggccgtgggtagagacgtgaatggaggaagtggagtagatgaaatggttaaatgttggagaaaagaggctatttatg
aatatgaccactgtcattcagataaaatttctggactgttatcattgaaaaaagtctcattatgtttctatttgaaagcaaaccattatgc
ttttttgaggaaaaaaaaaaactgtgagtcacgttatgcttgcaagtgtttaattcagaccatttcatctttaagaaggcccctggtcac
attatacggatgatttgcttattaaatggaactcctgtttcttgcaccatgttgtggggtcaatatgagaagcctaattaacagaataaa
aagcattaaagcttctttagctaaagtcaaacttagagaattgtctaatggtatgtagcccctcgttctaagatgggcgttttccccag
ataacttgaaaatctactggtaacagccacttccctttaaagaattctattactaatagccatgacaaaatggtattgtatttcaaagtt
aagaatttgcaggccttaaaaactaacttatttttcctgattattgagtttattgtagaattctacgtgtaagcattccccagccgctatag
ctttgaataagcagagcttttttcagagttctggtagcgcccagcccagcaccttttattctgaatgtgaagtgtgtgcctccgtgtcac
agagtcacagcctccccagggacgctgcgcgcggagccctgtcagagcagcgcgtcagtgacagcggcagccgagccagg
aagttatcaggcagcctcgaccaccaccagatttgactccgcgagctcttttgagggaaaacctggtaaaacgtcaaggtgtcta
actgacctcgcctttatcatctgttctgtaaatcttaggaaaggtctgggaaaaaatcaaaacgattctgtccgttaaagggcagcc
actcctggccctccaggatgccggggtctgagtgatcccgagctgatctgcagaagcacagcctgtggcatttgcggtttattgtca
tgaaaatgattcaacgtagaactttttcaaatggcaaaatcaaaccgctcttctttatattgtttttgaatgagttgtcatggaaacaaa
atggaaataaatggtgttttttttccagatttgtgctcattgcaggtcttcccaaaatagtagctttactgaatgaacaaagaactaaaa
tgaaggtcccaaactcatcgctaaggggcctccactaaagagcatcacccctggaggggcgcgggtctcagggtccttggccg
cgtgtggattatgtcaccacaggagagggacgagtcctttccaggcacatgaggaggaggaatcagtgttaatgggtggctttgc
atctgtgaaatcgcataaacttaagttagctgaagctgtcgtgagactggcatttccaaattggattgaaggtttcaggcttcatgcca
gcgcaccacagcctgttcctgagtatctgtgctgagaggctgtaagattagtgtgaacaggagaaatttccaggtaggcctctagc
ttcattaccgttgggtttcttactgccggtattcagacaggtagacatgactcgctggagtttgattgccttttcttacctcatgttggtaga
aacatcaatgagctgaaatgtatagggagataaaatgggcagaggcaggaggaaggaagaggaagcgccagcctgaggt
ggtcatgaactgcatactcagaccgtggctcatggggaattggttgccattgaccacgtgaagcagctccagcctccacgccagt
tgcatgttggttaaaagtttgtccttggtgcgataagtgtgtggaacgggagagagaccatctctgcctctgagattggattcgggttt
cagttcgttgtcggtaaagtagtgaagtgtggcaggggttctctgaagcctcagggtctacacaggcaccaccctgaggagcag
cctctgcagacggggcctgatctctgccagggcagtaggaagcatgacacgtcccgccagccaggccacagagctgaacact
gcctcctcccctgtccagGTCCCGCTCCCGGTCCCCTCGGAGGAGAGCCCACTCCCCTGAGAG
ACGGAGGGAAGAGAGGAGTGTGCCCACTGCCTACCGCGTGAGCCGCAGCCCTGGGG
CCAGTAGGAAGCGGACCCGCTCCAGgtaggccactgggtgtgcacgcaggtgctggatgtgggccaggtttcc
ctgggtggaaagggcgtctgaaggtcgggtatctgtgagcagagctgtggatgaccagagggaggtgctgagtcccccaccac
ccccccacccccagtggcatggccatcactgttgacacttgatcacactgagctcctgtgtctggtgggcgggggtcacttaccca
ccggggctctgcacggcctggcttcgtgtccagctttccactgtgctggtacctcggctgggtccacatgcagctgctgcccctctac
ctgctggtggagaggacaggaaggcacaaacagaaggaaaatgcaagcttccggtcctaaagcctcctggtctcaagggca
gtcactgtggttgcctggctgctgtgtgacggtgactacggcccaggctggagctcccaggagaggccacagagtcctgttgggg
cctagagggcagggagcatccatcgcttacctcttgaccactaaggagagcctgtcttggttggagcaggagatggagggaggt
tagcattcatgttcatcaagtagaagccccagccgtggtgcctggcagggcctctgacagcccagggtgccacgggctcacccc
tcactcagtgcctggcactcagtagaggttccacctttcacttcaggaaataggtccaccatctgtccgctcaccccggcttccagta
gctgtggacggccacctccattggtgccgccagtgagcactaccctctcggccgtgggggtgccatctcacgagcgcctcctctg
gttctcacccactgatgtcaccacccagtgccttgcgtggggcagccgtgcatttccactctttccaagcacaaggagcttgttttgtg
tccccatgtggagttcgtgcagcctcctggctgtgtgggtggaccgtgtctgcgtctggagctacacagagaaggatggagcattg
cacatcgtagccttgagcttcataacacggcactgctaagtgcatgggggtcaggacactcagggtcccagagccttccagagg
acgagccttacattgccaggatcacccacacactgggaccctcctgctcctgggacggatggtcccagccatcacccacactgc
ctagccacaaggcacacactaggcagagagccacagcaggtcctccccacagcaccctgggcaagaagaccgtgctgcgg
ttggcctagtaccacggttccctccgttgacaagatgtgatttttttcttaaaacagaaaaattagcaaaggaactataaagcggata
gataccagcaatgtttcatgtacacctggctctgtttataaattacattttgttccttagtaatcctacactgagcattcatgtctgctctcat
acaatctgatgaaaattaaaatgttagcatccatcccttaaacaagtaatttcacatcagaaattcaccatcacctttggtatatgtga
agggcatggttagaaattaattccgtctcaacagaagaggccttgctttgccttcacattaacctttgctttaagagagacctcgtgtg
agcaagtagtgattgtatctggaagtagcagcgtcctgatggccagccagcacactcagacgccagactcgcgtgacctgctga
cattctcaccgagcactaacaggtcacacaagagaagcaaagggttagactcagtgcagtgctgagccctgagctgccgtgcc
cagacagacggaattaaacctgcaaaccaaagtctgcggagtgttaaactgtgattcactaggaactcaatagaggtgaatacg
tgtgtaattactggttaattttgtattcttaattacaagcccccagttagtctataaatccagaatatgggtttggttttgttttcttttgggggc
gttttttttttttgagacagggtctcaccctgtttcccaggctggagtgcagtggcgtaatcacagctcactgcagcttctacctcctggg
ctcaagccatcctcccacctcagcctcctgagttgctggggccacaggctgtcaccaccatgcctggctggctgctctcaagctcct
ggcctcgagtgatcaacctgcctcaacctccaaaagtactgggattgcagacatgagctcccatgcctggtacagaatatgttttat
tagcaatcattatattaatcctacagccagcccgtgtccctgtctcagagcgggcgtccacttccttgctgtggcttagtgcacataatt
cagctaccaagttgctgtcactttaatgctgtgacagcaccagaccaaacccagggaaatgcccactaccgagatttgctgcttttt
ttctttttcttttttatttttatttgagatagggtctcactcccattgcgctggctggagtgcagtggcacaatctcagctcactgcggcctca
acctcctgggttcaactcgtcctcccacctcagcctcctgtgtagctgagactacaggcacatggaaccatgcccagctaattttttg
tatttttagtagagacagggttttgccatgttgcccaggatggtctcaaaatcctgagctcaagcagtctgcctatctcagcctcccaa
agtgctggcataaaccaccatgcccggccctgaagggtcatttctgtaaactgattattgcctgattctttcactgacttctcacttgga
aacttttttaacttataggcaagtttttaaaatagtacaatggggccagattcagtagctcacacctataatcccagcacttggaggc
caagatagcaggatcacttgagctcaggagttggaggctgtagtgtgggctgtgatcgtgcctgtgaatagccactgcacccccc
acctgggtaacagagtgaaaccctctctttcaaaaaaaagtgtacaataaacacccatatgcataaaatctgtagctcagttccac
aagagctgacattttgccacattgctctctctcaccccttcccatcccgcccatcccatccactcccctccctccctcctccgttcgtg
gtgtatttcatgaccttggcattcctgagaattccaggccagctccactatagatggtcccacagttgggcttcgtcttgctgtgtcccc
gtggctgggttcagggcaaatgttttggctgcgtaggcgacattgcgtagcttcccattgcatcacagatcaggacacacagaagt
gtccatttgtcccatcattcatgatgctaagtttgaccacttgattaagtctgcatctgccccttcgtctccccaccagcgaggaatcca
ggaggtgacactgaagcagcgcggctctcctgctcccagcagctgtcttctcatttgtctcagcatccctgggtgacccctgcctga
atcagttcttacactgctgactgcaaaatagtgactttccccctctcttcttccttctgtgtttatgctgaagaccctgcccctttgtttaaat
ctcaccgtggactcaggagcatttttggttttgattttttatttgttgtgtgataatccattgctattattattctattagatggtgacattgtctc
cagtttggccagtggcaacccttccaagtcagttctgttcttttgacacctcccatagttctttgcattcttgcgtttggtacaagatgttcc
aggtttactgggcattttccctgctccagccctggaatctaccatttcttcaaggacctctggttccttttagtgaatatttgaaaatccag
atgtggacgtatgaggaatttttaggagtaaaatttggtacagtgtggaaatatataaaacaacattcatgaaagttattttgagtatg
tcataaaagtgtttttcagccaggcacaatggcgggcacctacagccgcagctacttggagggctgagtggatctcttgagcctag
gagttcacatccagggcttttcacaagaatattgaccaaatcttctggtagcacacttcaacaagatgtcccggttatcttattgtagc
aaatacaatgaatgattagttacaagtttttcccattgagtttctagtacttaacactgcacgaggcacatggacaactgtttgttgagt
gagtgaatgggagttcactgctgcagtaaagatctgcctttatacatgaaatgttaattccaggtagactttgctaagcgaaggatg
cataacctaattccctagagcaaccactaaaaacaaaaatgtagctaaaaagccaatagcagatataaagtaggattctagatg
ctttcttaaattcatgaaacagcagaaaagggcaggtgggggaaagaacaaatgggacaaataaaaacaagattgtagactt
aaaaccatctgtaaaataattacattaaatgtaagaagactaaagactagttaaaaggcagtgattgtggagtggattaaagagc
aagacctggcctggcgcggtggctcatacctgtaatctcagcacttcaggaggccaaggcaggtggatcacctggggtcagga
gttcaagaccagcctggccaacatggtgaaaccccgtcactactaaaaatataaaaattaggtgtggtggcaagtgcctgtaatc
ccagctactcgggaggctgaggcaggagaattgcttgaacctgggaggcggaggctgcagtgagccaagatcgtgccactgc
actccagcctgggtgacaaagtgagactctatctcaaagaaaaataaacgaaacttttccaccaaactccagtcccagatggctt
caccagtgaattctaacattcaagaaaggaggggccaggcacgatggttcacatctgtaatcccagcacttcaggaggctgag
gcaggtggatcacgaggtcaggagtttgagaccagtctggccaacatagtgaaactctgtctctactataagtacaaaaaattaa
ccgggtgtggtagtgtgcgtctgtaatcccagctacctgggaggctgaggcaggagaataacttgaactcgggaggcggaggtt
gcagtgagccaagattgcgttccagcccgcgacagtgcaagactccgtctcaaaaaacaaaaagaaagaaagaagggata
ctcttttttaaaaaatagatgaaggaacacttcccatctcatctcttgagtccatcataactctcatacctaagccagataaggattctg
tgtttgggggagggggtgtgcacatgcacccttgtctgttcacagatcagtactgtgtgcacccgtgtgtgttcacggatcagtactgt
gtgcacacgtgtgtgttcactggtcattactgtgtgtgcacccgtgtgtgtgcacagaccagtacagtgtgtgcactcgtgtgtgttcac
ggatcagtactgtgtgtgtgcacgtgtgtgttcacggatcattactgtgtgtgcgcccatgtgtgttcacggatcagtactgtgtgtgtgc
acgtgtgtgttcacggatcgttactgtgtgtgcacccgtgtgtgttcacagatcattactgtgtgtgcgcccgtgtgtgttcacggatcat
tactgtgtgtgcgcccgtgtgtgttcacagaccagtactgtgtgtgcatatgtgtgtattcacagatcagtactgtgtgtgcacccgtgt
gtgttcacagaccagtactgtgtgtgcatatgtgtgtgttcacagatcagtactgtgtgtgcgcccgtgtgtgttcacagatcagtactg
gtgtgcatgtgtgtgctcacagaccagtactgtgtgtgcatatgtgtgtgttcacagatcagtactgtgtgtgcacccgtgtgtgttcac
agaccagtactgtgtgtgcatacgtgtgtgttcacagatcagtactgtgtgtgcgcccgtgtgtgttcacagatcagtactggtgtgca
tgcgtgttaacagaccagtgctgtgtgtgcacatgtgtgttcacagatcagtactggtgcacatgcatgtgtgttcacagaccagtgc
tgtgtgtgcccataagtatatgttcacagaccaggactctcaagaacatagatgcaaaaatacttcacaaaatattagccaactaa
gtattactgagactcctgttctccacaagttgacgcagagatgcagtgcagtcccactcagagctcccacggcttttctagaaattg
gcacacaaactccaaagcgtgtgtggaaatgcagatgacctgggagacccaaaacaacctccttgacaaagagcaggatttc
aagacttaccagaaagctacagtaaccaaggcagtgtggtgtcagcatgaggatacaatagagcagtgggatggaatagaaa
gtacagaaaaaaaattccatacccaaagggcagggggccgggaccacagccacagcgattcagtgaggaaaaagagaaa
ggaaagtcttttttttttttgagacagggtctcactctgttgcccaagctggagtgcagcagtggtgtgatctcgactcagcccggctg
actgcagcctcctgggctcaaggaatcctcccacctcagctgggaccacaggcacacaccaccatgcccagctaatttttttttatt
gtgtgtagagacagggtctcgctatgttgcccaggctgatgttgaactcccaggctcaagcagtcctcctaccttggcctccgaaa
atgctgtgactgcaggcatgagccacagcacccagccaggaaactctttccaacaaaacttgcatgaacagctggatatcgga
atggggaaaaagtgcactgcatgctgtatgcaaaatttaattcagggcggatcagagatctaaacaaaaactagaaccattaag
ctttttgaagaaaacacagaatatgttcatgaatttgagggtggcaaagattccttaagatgtagaaactcctctgataagaggaaa
aaaccaattagacttcattgaagtttaaaaacttctctcaaaaggcacagttaagaagatgaataggcaggccgcaggctttgctg
catgtgtctctgacaaaagcctgtgtcagtaccaaaaagacaaaggacccaattagaagggggcagatgaagccagccgact
tgacagaaggatctcttaaatagccggtacacacatggaaagatgtggaacggcatgagtcaccagtcagggacgtgctgatg
caaccaacgagacaggactagacgggggtcacccgtccctaaaaaccaggacgggctcgggggagagtgggcacgggcc
cagcggctgcgctctcagacactggattgggaaacgtgtgcagtttcttgtgacgttaagtacacacctactccctgaccagctgtc
ctgttcctagctgtgaactcctctataaagtcaacatttaaccaaaaacactttgattcataattaccgaaaactggaaacaaccaa
atctctattaacaggagaatgaatcaacagataatggtagcgtcctgtcctgtaatactattcatcggtaaaaggaacaaattgag
gatcaccctgcgtcgtggaggagtctcagacatgctttgctgagcaaaagcagccagacacaggccagccacagtggctcac
acctgtgatcccagcactttgggaggccaaggcaggaggattgcttgagcccaggatttgcaggctttttttttttttggtagagaccc
ccatctctacttaaaaaaaaaaaaaattagccatttgtggtggcgtctgcctgtcgtcccagctacttgggaggctgaggcaagag
gatcactggagcctgggaggtcaaggctacagtgagcagggattatgcccctgcactccagtttgggcaacagagggaaactg
agaaacaaacaacagaaaaccaagaagccaaaccaacaaacaaacacagacatagcgtggggtttgtctacatagagcttt
aagctgtgtcctagaaaccagagcagtggggaacgctgagggtggagaaggggtatagacggacttgaagtggcattgagga
gccttctggaatgaagggacgcccctgcgtggataaggcccaggtgtcagggtgtgcgcacttgccaagctcagcggcagcac
cgaggacagcgtttcacccaatggacagtggcacctcggtgctttaaaaaaaaatgaatgagttgctccattccttcagcaaggg
cttagatcagattgtagcagaattgaaccagtttgcagttaaggattagtaacctgccttttgttcattatgcagccacataaactcag
ctggatttggggagtaagtcattttggacacatgtcacatgctggtatatgttttatttatttgccgcttcctttgaaatcctggcatgtgttt
acagacaacaatttcacaaaacattttgcagtttagaaaaatgactctttcgtgcaggtcccacatgcgtgtgttgaacagtaaaca
acatgttgtcctcactgggcacgtcaggcaggcttccagaagatgccaagtcatctgcccgggcccagctcaccagggacagc
ccctccagcagctggatttaagctgccagcgagcaccgtctctggcaggtcccgccttgtttgaatggagctgggtgggagcgcc
acaggtctggcgctgctgcttaggtcacttcactggcaccaacacagtctgctcacgcccagaaccacacaagggagcccgga
cagaaacgctcagtccccccctgcatatcggggctgtccctaccagggcatgctgtggtccctggctaccgcagctctgtctaagtt
ctgcagggccagacactggtgaggtcctagagatgggtagagggcacagcccctcgatggggtctgcaccccagactctgag
cacagccccagccattaagcaagaatgtcccagatatcggggggtggcacaagaaatgcatgaagtccggaggccctgatga
ggggcagggcttggggtaactgggcctgtgcacaggccctggaggtctccctggaaggcagaggaggccaggctgggaagg
ggcttcgtggcacgcagaatcataagggaggccagacgcttgcagctgtgcaaatagcaaccccaggagagagtcagacac
cagcagagaaccacggttcccccttcaggttggcacattgagcagtttgggtccacctggataacgagcgtgaggctgagccag
ggagtccccctggcagcttctgcagcagagggccccgcagccctactcctgggatctgtcctgcccaggcaccagcaagcagg
acgggaggggagggataggggaggggaggggagagggggaggggaggggaggggagagggggaggggaggggag
gggagcggagagggggaggggaggggagggaaggaaggaaggaaggaaatcagtgatgcaaatgacccatgcaaaga
ctctccaagaaacactgtactcagggccagaagcgcaggctgcagcgtctgttacagacgaattctgaaagaagatgccaggt
agggcacctcagggcctggagggcctcacaggaagggctcaggcctgtctgcctttaccaagtacatgttcactctcttaggtgttt
gtaggggagtggccaagacagccacgtggctcaggtgtggaatgaagctagaccaggtggaagccgaagggtcggcctctc
caggcaggagagaaggatgatctaagggcaggtgcaggccagaatgtctggaaagcatttctggtgcgggattgccagtttggt
gacgtggactctgggaagcaaggggacaggggacagcagtcagagctgagctgctgcccacagagcaggctccactgccc
agaggctaagcggtatcaccaagcggcggacaactggcaggtcaggaagaagtgccactccagcctggacaacagagtga
gaccccatctcttaagaaaaaggaagaagcagcaccagaagctgcgccccctagtcttaactgtctgggaggctgaggcagg
aggttgcttgaggtcaggaggtgaaggctgcagtgagctgtaatggcaccactgcactccagcctggacagcagcacgagacc
ttgtctgttttttcaaaaaaaaggaacactaaactttgatgtattgatactttaataaatttcctgtatctttttggaaatttttattgatgaaa
cataagtggcaaagcactatgaactgcctgtggtggcttatcttaggtattttacatgtaaataaaatgctggttgcatcttaaatacc
acaaatattttacttgaggtcctaaatggggacgcgtcatctgttatcagttaaatgaaataagtagctttaagagaagttaatgggtt
tggagtggttccgtccctgaattgtgccttgatgaactcttagccaaaaactggctcagatccgagcttctccctttgtgccctgccttta
aaccaaagctgcatctctcacagaaactcttgcctttcagAAGTCCCCACGAGAAGAAGAAGAAGAGGCGG
TCCCGGTCGCGGACCAAGTCCAAGGCCAGGTCTCAGTCGGTGTCACCCAGCAAGCAG
GCAGCGCCCCGGCCCGCGGCCCCCGCGGCCCACTCGGCGCACTCAGCCAGCGTCTC
CCCTGTGGAGAGTCGGGGCTCCAGCCAGGAGCGCTCCAGgtaacccctgtcctccagcagctctctc
tggggaaaggcaaggggcggccagcaggactctccctcctccctgagtccttgcctatgtcagtactcgcctgtgtccagggggc
gccagccacaaagccaaaccgcaccccctctagcaaggaagtcgccctagatgtggcttctcacaatccatgagcgctcaga
ggagcaggtcctgtactggggagaccctcctgcagagcccaggagtggagcagtccacttgaagcagcccaagtgtcacaca
cgtgcctgatgcccaccaggcacactgggctgtgcaatgaccagtagaccgggaactgtcaccaggtccccaggctgccgtgg
ctggagcaggtccccaggctgcaacggccagggccaaatgacgccaacctgtcaccgggcatcacacctgggcagcagca
cagacgtgggcgtcccagtcccgggctaggtgataatgacttcaagtcagacaccctccgctgcccaggcacccacaccctgg
ggggaccagagagggcagcatctgggaacagctgctccctttaaactgattgcttccataaatgtcaatcatgggagtaacgcg
caactgttccattctagtggcagaggcctcagctaatttgagatggattagaatctaagaggtggcacctttagagttaaaatgtaa
atcaggctgggcgccgtggctcatacctgtaatcccagcactttgggaggccagggcaggaatttgagaccagtctggacaaca
tggcaggaccttgtctctactaaaaataggtggcacgcgtctgtaatcccagctactcaggaggctaaggtgagaggattgcttga
gcccaggaggtggaggctgctgtgagccatgacggcaccactgcacatcagcctgggtgacagagagagaccctgtttctgaa
aatgtaataatgataaaatgtacatcagtgtaggaggctgagcatcgctgcggggagggggtgttggctccagcacacagacg
cctcatgcacaggccgagggcacctacagccaaggccgtggttctgggaaggctccaccgttctgctgagtctttcctttctttgtttc
ttttttcctttgtgtttaaggtaattttatatgaaaatctttttgagttagattgcaatttgtaaacatttcagatgagtataacacagcatgttt
atgatgccaagttttattgaaggatactggaggggtgggcgcggcggctcacgcctataatcccagcactttgggaggccaagg
cgggtggatcacctgaggtcaggagttcgagaccaccctgaccaatatggtgaaaccccgtccctactgaaaatacaaaaatta
gccgggcatggtggcacacgcctgcaatcccagctactcaggaggctgaggcaggagaattgcttgaatctgggaggcagaa
gttgcagtgagctgagaacgtgccattgcactccagcctgggtgacagagtgaaactcttgtctggaaaaaaaaaaaaagatac
tggaagcagatgcagtgggcacttctcagttctagagttggggttcggaggtggggatgctgttcactggccttggctcagcatcttc
acacggttgtaagctctgctctctctctctctgcattagGGGAGTCTCTCAGGAAAAAGAAGCCCAGATCTCT
TCAGCAATCGTTTCTTCCGTGCAGAGCAAAATCACTCAGgtcagtgggcacgcccccctcccgctccc
agcctttcatcaaggggcctcgtggtttctctgttgctaattttcattccctgtccctcctgtccctgtcatgggacagggatctcgggca
aaataccacaggctctgggtgaggccgagggcaaagccgtgtggcccgcaccctgcacagccaggctcctccgccgccccc
acggtgctagcaccgtctggtcttgaccaccaactcgttgatgaatttcttcaccacgtgggttgtctggccaggtcttcacaggttctc
ctctgtgtctcgccctgcacagGATCTCATGGCCAAAGTCAGAGCGATGCTTGCAGCTTCCAAAAA
CCTGCAAACCAGCGCTTCCTGAGACGGGGCCAGCGGAGGCAGAGCCGGGAGGCTGC
GTGGGCTTCTGGGCAGGCTCACGCAGACGCCGGCCACACCATCCACCTGGCCGCCTC
CATGGACCCTTGGTGGCTTTTGTAAATTAATTTTTGATGACATTTTGAGTTTTAAGATTTC
TGACCAGCAGTCTCTTACCTGTATATTTGTAAATATATCATGTTTCTGTGAAAATGTATTA
TGAAATAAAATGGGAGGAAACACCTTTTCTAGCTAG
SLAMF1 coding sequence

SEQ ID NO: 10

ATGGATCCCAAGGGGCTCCTCTCCTTGACCTTCGTGCTGTTTCTCTCCCTGGCTTTTGG
GGCAAGCTACGGAACAGGTGGGCGCATGATGAACTGCCCAAAGATTCTCCGGCAGTTG
GGAAGCAAAGTGCTGCTGCCCCTGACATATGAAAGGATAAATAAGAGCATGAACAAAAG
CATCCACATTGTCGTCACAATGGCAAAATCACTGGAGAACAGTGTCGAGAACAAAATAG
TGTCTCTTGATCCATCCGAAGCAGGCCCTCCACGTTATCTAGGAGATCGCTACAAGTTT
TATCTGGAGAATCTCACCCTGGGGATACGGGAAAGCAGGAAGGAGGATGAGGGATGG
TACCTTATGACCCTGGAGAAAAATGTTTCAGTTCAGCGCTTTTGCCTGCAGTTGAGGCT
TTATGAGCAGGTCTCCACTCCAGAAATTAAAGTTTTAAACAAGACCCAGGAGAACGGGA
CCTGCACCTTGATACTGGGCTGCACAGTGGAGAAGGGGGACCATGTGGCTTACAGCTG
GAGTGAAAAGGCGGGCACCCACCCACTGAACCCAGCCAACAGCTCCCACCTCCTGTCC
CTCACCCTCGGCCCCCAGCATGCTGACAATATCTACATCTGCACCGTGAGCAACCCTAT
CAGCAACAATTCCCAGACCTTCAGCCCGTGGCCCGGATGCAGGACAGACCCCTCAGAA
ACAAAACCATGGGCAGTGTATGCTGGGCTGTTAGGGGGTGTCATCATGATTCTCATCAT
GGTGGTAATACTACAGTTGAGAAGAAGAGGTAAAACGAACCATTACCAGACAACAGTGG
AAAAAAAAAGCCTTACGATCTATGCCCAAGTCCAGAAACCAGGTCCTCTTCAGAAGAAA
CTTGACTCCTTCCCAGCTCAGGACCCTTGCACCACCATATATGTTGCTGCCACAGAGCC
TGTCCCAGAGTCTGTCCAGGAAACAAATTCCATCACAGTCTATGCTAGTGTGACACTTC
CAGAGAGCTGA
CD86 coding sequence

SEQ ID NO: 11

AGGAGCCTTAGGAGGTACGGGGAGCTCGCAAATACTCCTTTTGGTTTATTCTTACCACC
TTGCTTCTGTGTTCCTTGGGAATGCTGCTGTGCTTATGCATCTGGTCTCTTTTTGGAGCT
ACAGTGGACAGGCATTTGTGACAGCACTATGGGACTGAGTAACATTCTCTTTGTGATGG
CCTTCCTGCTCTCTGGTGCTGCTCCTCTGAAGATTCAAGCTTATTTCAATGAGACTGCA
GACCTGCCATGCCAATTTGCAAACTCTCAAAACCAAAGCCTGAGTGAGCTAGTAGTATT
TTGGCAGGACCAGGAAAACTTGGTTCTGAATGAGGTATACTTAGGCAAAGAGAAATTTG
ACAGTGTTCATTCCAAGTATATGGGCCGCACAAGTTTTGATTCGGACAGTTGGACCCTG
AGACTTCACAATCTTCAGATCAAGGACAAGGGCTTGTATCAATGTATCATCCATCACAAA
AAGCCCACAGGAATGATTCGCATCCACCAGATGAATTCTGAACTGTCAGTGCTTGCTAA
CTTCAGTCAACCTGAAATAGTACCAATTTCTAATATAACAGAAAATGTGTACATAAATTTG
ACCTGCTCATCTATACACGGTTACCCAGAACCTAAGAAGATGAGTGTTTTGCTAAGAAC
CAAGAATTCAACTATCGAGTATGATGGTaTTATGCAGAAATCTCAAGATAATGTCACAGA
ACTGTACGACGTTTCCATCAGCTTGTCTGTTTCATTCCCTGATGTTACGAGCAATATGAC
CATCTTCTGTATTCTGGAAACTGACAAGACGCGGCTTTTATCTTCACCTTTCTCTATAGA
GCTTGAGGACCCTCAGCCTCCCCCAGACCACATTCCTTGGATTACAGCTGTACTTCCAA
CAGTTATTATATGTGTGATGGTTTTCTGTCTAATTCTATGGAAATGGAAGAAGAAGAAGC
GGCCTCGCAACTCTTATAAATGTGGAACCAACACAATGGAGAGGGAAGAGAGTGAACA
GACCAAGAAAAGAGAAAAAATCCATATACCTGAAAGATCTGATGAAGCCCAGCGTGTTT
TTAAAAGTTCGAAGACATCTTCATGCGACAAAAGTGATACATGTTTTTAATTAAAGAGTA
AAGCCCATACAAGTATTCATTTTTTCTACCCTTTCCTTTGTAAGTTCCTGGGCAACCTTTT
TGATTTCTTCCAGAAGGCAAAAAGACATTACCATGAGTAATAAGGGGGCTCCAGGACTC
CCTCTAAGTGGAATAGCCTCCCTGTAACTCCAGCTCTGCTCCGTATGCCAAGAGGAGA
CTTTAATTCTCTTACTGCTTCTTTTCACTTCAGAGCACACTTATGGGCCAAGCCCAGCTT
AATGGCTCATGACCTGGAAATAAAATTTAGGACCAATA
CD83 coding sequence

SEQ ID NO: 12

ATGTCGCGCGGCCTCCAGCTTCTGCTCCTGAGCTGCGCCTACAGCCTGGCTCCCGCG
ACGCCGGAGGTGAAGGTGGCTTGCTCCGAAGATGTGGACTTGCCCTGCACCGCCCCC
TGGGATCCGCAGGTTCCCTACACGGTCTCCTGGGTCAAGTTATTGGAGGGTGGTGAAG
AGAGGATGGAGACACCCCAGGAAGACCACCTCAGGGGACAGCACTATCATCAGAAGG
GGCAAAATGGTTCTTTCGACGCCCCCAATGAAAGGCCCTATTCCCTGAAGATCCGAAAC
ACTACCAGCTGCAACTCGGGGACATACAGGTGCACTCTGCAGGACCCGGATGGGCAG
AGAAACCTAAGTGGCAAGGTGATCTTGAGAGTGACAGGATGCCCTGCACAGCGTAAAG
AAGAGACTTTTAAGAAATACAGAGCGGAGATTGTCCTGCTGCTGGCTCTGGTTATTTTC
TACTTAACACTCATCATTTTCACTTGTAAGTTTGCACGGCTACAGAGTATCTTCCCAGAT
TTTTCTAAAGCTGGCATGGAACGAGCTTTTCTCCCAGTTACCTCCCCAAATAAGCATTTA
GGGCTAGTGACTCCTCACAAGACAGAACTGGTATGA
HRH1 coding sequence

SEQ ID NO: 13

ATGAGCCTCCCCAATTCCTCCTGCCTCTTAGAAGACAAGATGTGTGAGGGCAACAAGAC
CACTATGGCCAGCCCCCAGCTGATGCCCCTGGTGGTGGTCCTGAGCACTATCTGCTTG
GTCACAGTAGGGCTCAACCTGCTGGTGCTGTATGCCGTACGGAGTGAGCGGAAGCTCC
ACACTGTGGGGAACCTGTACATCGTCAGCCTCTCGGTGGCGGACTTGATCGTGGGTGC
CGTCGTCATGCCTATGAACATCCTCTACCTGCTCATGTCCAAGTGGTCACTGGGCCGTC
CTCTCTGCCTCTTTTGGCTTTCCATGGACTATGTGGCCAGCACAGCGTCCATTTTCAGT
GTCTTCATCCTGTGCATTGATCGCTACCGCTCTGTCCAGCAGCCCCTCAGGTACCTTAA
GTATCGTACCAAGACCCGAGCCTCGGCCACCATTCTGGGGGCCTGGTTTCTCTCTTTTC
TGTGGGTTATTCCCATTCTAGGCTGGAATCACTTCATGCAGCAGACCTCGGTGCGCCG
AGAGGACAAGTGTGAGACAGACTTCTATGATGTCACCTGGTTCAAGGTCATGACTGCCA
TCATCAACTTCTACCTGCCCACCTTGCTCATGCTCTGGTTCTATGCCAAGATCTACAAG
GCCGTACGACAACACTGCCAGCACCGGGAGCTCATCAATAGGTCCCTCCCTTCCTTCT
CAGAAATTAAGCTGAGGCCAGAGAACCCCAAGGGGGATGCCAAGAAACCAGGGAAGG
AGTCTCCCTGGGAGGTTCTGAAAAGGAAGCCAAAAGATGCTGGTGGTGGATCTGTCTT
GAAGTCACCATCCCAAACCCCCAAGGAGATGAAATCCCCAGTTGTCTTCAGCCAAGAG
GATGATAGAGAAGTAGACAAACTCTACTGCTTTCCACTTGATATTGTGCACATGCAGGC
TGCGGCAGAGGGGAGTAGCAGGGACTATGTAGCCGTCAACCGGAGCCATGGCCAGCT
CAAGACAGATGAGCAGGGCCTGAACACACATGGGGCCAGCGAGATATCAGAGGATCA
GATGTTAGGTGATAGCCAATCCTTCTCTCGAACGGACTCAGATACCACCACAGAGACAG
CACCAGGCAAAGGCAAATTGAGGAGTGGGTCTAACACAGGCCTGGATTACATCAAGTT
TACTTGGAAGAGGCTCCGCTCGCATTCAAGACAGTATGTATCTGGGTTGCACATGAACC
GCGAAAGGAAGGCCGCCAAACAGTTGGGTTTTATCATGGCAGCCTTCATCCTCTGCTG
GATCCCTTATTTCATCTTCTTCATGGTCATTGCCTTCTGCAAGAACTGTTGCAATGAACA
TTTGCACATGTTCACCATCTGGCTGGGCTACATCAACTCCACACTGAACCCCCTCATCT
ACCCCTTGTGCAATGAGAACTTCAAGAAGACATTCAAGAGAATTCTGCATATTCGCTCC-
TAA
IL-2 coding sequence

SEQ ID NO: 14

ATGTACAGGATGCAACTCCTGTCTTGCATTGCACTAAGTCTTGCACTTGTCACAAACAGT
GCACCTACTTCAAGTTCTACAAAGAAAACACAGCTACAACTGGAGCATTTACTGCTGGA
TTTACAGATGATTTTGAATGGAATTAATAATTACAAGAATCCCAAACTCACCAGGATGCT
CACATTTAAGTTTTACATGCCCAAGAAGGCCACAGAACTGAAACATCTTCAGTGTCTAGA
AGAAGAACTCAAACCTCTGGAGGAAGTGCTAAATTTAGCTCAAAGCAAAAACTTTCACTT
AAGACCCAGGGACTTAATCAGCAATATCAACGTAATAGTTCTGGAACTAAAGGGATCTG
AAACAACATTCATGTGTGAATATGCTGATGAGACAGCAACCATTGTAGAATTTCTGAACA
GATGGATTACCTTTTGTCAAAGCATCATCTCAACACTGACTTGA
TRL7 coding sequence

SEQ ID NO: 15

ATGGTGTTTCCAATGTGGACACTGAAGAGACAAATTCTTATCCTTTTTAACATAATCCTA
ATTTCCAAACTCCTTGGGGCTAGATGGTTTCCTAAAACTCTGCCCTGTGATGTCACTCT
GGATGTTCCAAAGAACCATGTGATCGTGGACTGCACAGACAAGCATTTGACAGAAATTC
CTGGAGGTATTCCCACGAACACCACGAACCTCACCCTCACCATTAACCACATACCAGAC
ATCTCCCCAGCGTCCTTTCACAGACTGGACCATCTGGTAGAGATCGATTTCAGATGCAA
CTGTGTACCTATTCCACTGGGGTCAAAAAACAACATGTGCATCAAGAGGCTGCAGATTA
AACCCAGAAGCTTTAGTGGACTCACTTATTTAAAATCCCTTTACCTGGATGGAAACCAGC
TACTAGAGATACCGCAGGGCCTCCCGCCTAGCTTACAGCTTCTCAGCCTTGAGGCCAA
CAACATCTTTTCCATCAGAAAAGAGAATCTAACAGAACTGGCCAACATAGAAATACTCTA
CCTGGGCCAAAACTGTTATTATCGAAATCCTTGTTATGTTTCATATTCAATAGAGAAAGA
TGCCTTCCTAAACTTGACAAAGTTAAAAGTGCTCTCCCTGAAAGATAACAATGTCACAGC
CGTCCCTACTGTTTTGCCATCTACTTTAACAGAACTATATCTCTACAACAACATGATTGC
AAAAATCCAAGAAGATGATTTTAATAACCTCAACCAATTACAAATTCTTGACCTAAGTGG
AAATTGCCCTCGTTGTTATAATGCCCCATTTCCTTGTGCGCCGTGTAAAAATAATTCTCC
CCTACAGATCCCTGTAAATGCTTTTGATGCGCTGACAGAATTAAAAGTTTTACGTCTACA
CAGTAACTCTCTTCAGCATGTGCCCCCAAGATGGTTTAAGAACATCAACAAACTCCAGG
AACTGGATCTGTCCCAAAACTTCTTGGCCAAAGAAATTGGGGATGCTAAATTTCTGCATT
TTCTCCCCAGCCTCATCCAATTGGATCTGTCTTTCAATTTTGAACTTCAGGTCTATCGTG
CATCTATGAATCTATCACAAGCATTTTCTTCACTGAAAAGCCTGAAAATTCTGCGGATCA
GAGGATATGTCTTTAAAGAGTTGAAAAGCTTTAACCTCTCGCCATTACATAATCTTCAAA
ATCTTGAAGTTCTTGATCTTGGCACTAACTTTATAAAAATTGCTAACCTCAGCATGTTTA
ACAATTTAAAAGACTGAAAGTCATAGATCTTTCAGTGAATAAAATATCACCTTCAGGAGA
TTCAAGTGAAGTTGGCTTCTGCTCAAATGCCAGAACTTCTGTAGAAAGTTATGAACCCC
AGGTCCTGGAACAATTACATTATTTCAGATATGATAAGTATGCAAGGAGTTGCAGATTCA
AAAACAAAGAGGCTTCTTTCATGTCTGTTAATGAAAGCTGCTACAAGTATGGGCAGACC
TTGGATCTAAGTAAAAATAGTATATTTTTTGTCAAGTCCTCTGATTTTCAGCATCTTTCTT
TCCTCAAATGCCTGAATCTGTCAGGAAATCTCATTAGCCAAACTCTTAATGGCAGTGAAT
TCCAACCTTTAGCAGAGCTGAGATATTTGGACTTCTCCAACAACCGGCTTGATTTACTCC
ATTCAACAGCATTTGAAGAGCTTCACAAACTGGAAGTTCTGGATATAAGCAGTAATAGC
CATTATTTTCAATCAGAAGGAATTACTCATATGCTAAACTTTACCAAGAACCTAAAGGTTC
TGCAGAAACTGATGATGAACGACAATGACATCTCTTCCTCCACCAGCAGGACCATGGAG
AGTGAGTCTCTTAGAACTCTGGAATTCAGAGGAAATCACTTAGATGTTTTATGGAGAGAA
GGTGATAACAGATACTTACAATTATTCAAGAATCTGCTAAAATTAGAGGAATTAGACATC
TCTAAAAATTCCCTAAGTTTCTTGCCTTCTGGAGTTTTTGATGGTATGCCTCCAAATCTAA
AGAATCTCTCTTTGGCCAAAAATGGGCTCAAATCTTTCAGTTGGAAGAAACTCCAGTGT
CTAAAGAACCTGGAAACTTTGGACCTCAGCCACAACCAACTGACCACTGTCCCTGAGAG
ATTATCCAACTGTTCCAGAAGCCTCAAGAATCTGATTCTTAAGAATAATCAAATCAGGAG
TCTGACGAAGTATTTTCTACAAGATGCCTTCCAGTTGCGATATCTGGATCTCAGCTCAAA
TAAAATCCAGATGATCCAAAAGACCAGCTTCCCAGAAAATGTCCTCAACAATCTGAAGAT
GTTGCTTTTGCATCATAATCGGTTTCTGTGCACCTGTGATGCTGTGTGGTTTGTCTGGT
GGGTTAACCATACGGAGGTGACTATTCCTTACCTGGCCACAGATGTGACTTGTGTGGG
GCCAGGAGCACACAAGGGCCAAAGTGTGATCTCCCTGGATCTGTACACCTGTGAGTTA
GATCTGACTAACCTGATTCTGTTCTCACTTTCCATATCTGTATCTCTCTTTCTCATGGTGA
TGATGACAGCAAGTCACCTCTATTTCTGGGATGTGTGGTATATTTACCATTTCTGTAAGG
CCAAGATAAAGGGGTATCAGCGTCTAATATCACCAGACTGTTGCTATGATGCTTTTATTG
TGTATGACACTAAAGACCCAGCTGTGACCGAGTGGGTTTTGGCTGAGCTGGTGGCCAA
ACTGGAAGACCCAAGAGAGAAACATTTTAATTTATGTCTCGAGGAAAGGGACTGGTTAC
CAGGGCAGCCAGTTCTGGAAAACCTTTCCCAGAGCATACAGCTTAGCAAAAAGACAGT
GTTTGTGATGACAGACAAGTATGCAAAGACTGAAAATTTTAAGATAGCATTTTACTTGTC
CCATCAGAGGCTCATGGATGAAAAAGTTGATGTGATTATCTTGATATTTCTTGAGAAGCC
CTTTCAGAAGTCCAAGTTCCTCCAGCTCCGGAAAAGGCTCTGTGGGAGTTCTGTCCTTG
AGTGGCCAACAAACCCGCAAGCTCACCCATACTTCTGGCAGTGTCTAAAGAACGCCCT
GGCCACAGACAATCATGTGGCCTATAGTCAGGTGTTCAAGGAAACGGTCTAG
TRL8 isoform1 coding sequence

SEQ ID NO: 16

ATGGAAAACATGTTCCTTCAGTCGTCAATGCTGACCTGCATTTTCCTGCTAATATCTGGT
TCCTGTGAGTTATGCGCCGAAGAAAATTTTTCTAGAAGCTATCCTTGTGATGAGAAAAAG
CAAAATGACTCAGTTATTGCAGAGTGCAGCAATCGTCGACTACAGGAAGTTCCCCAAAC
GGTGGGCAAATATGTGACAGAACTAGACCTGTCTGATAATTTCATCACACACATAACGA
ATGAATCATTTCAAGGGCTGCAAAATCTCACTAAAATAAATCTAAACCACAACCCCAATG
TACAGCACCAGAACGGAAATCCCGGTATACAATCAAATGGCTTGAATATCACAGACGGG
GCATTCCTCAACCTAAAAAACCTAAGGGAGTTACTGCTTGAAGACAACCAGTTACCCCA
AATACCCTCTGGTTTGCCAGAGTCTTTGACAGAACTTAGTCTAATTCAAAACAATATATA
CAACATAACTAAAGAGGGCATTTCAAGACTTATAAACTTGAAAAATCTCTATTTGGCCTG
GAACTGCTATTTTAACAAAGTTTGCGAGAAAACTAACATAGAAGATGGAGTATTTGAAAC
GCTGACAAATTTGGAGTTGCTATCACTATCTTTCAATTCTCTTTCACACGTGCCACCCAA
ACTGCCAAGCTCCCTACGCAAACTTTTTCTGAGCAACACCCAGATCAAATACATTAGTG
AAGAAGATTTCAAGGGATTGATAAATTTAACATTACTAGATTTAAGCGGGAACTGTCCGA
GGTGCTTCAATGCCCCATTTCCATGCGTGCCTTGTGATGGTGGTGCTTCAATTAATATA
GATCGTTTTGCTTTTCAAAACTTGACCCAACTTCGATACCTAAACCTCTCTAGCACTTCC
CTCAGGAAGATTAATGCTGCCTGGTTTAAAAATATGCCTCATCTGAAGGTGCTGGATCT
TGAATTCAACTATTTAGTGGGAGAAATAGCCTCTGGGGCATTTTTAACGATGCTGCCCC
GCTTAGAAATACTTGACTTGTCTTTTAACTATATAAAGGGGAGTTATCCACAGCATATTA
ATATTTCCAGAAACTTCTCTAAACTTTTGTCTCTACGGGCATTGCATTTAAGAGGTTATGT
GTTCCAGGAACTCAGAGAAGATGATTTCCAGCCCCTGATGCAGCTTCCAAACTTATCGA
CTATCAACTTGGGTATTAATTTTATTAAGCAAATCGATTTCAAACTTTTCCAAAATTTCTC
CAATCTGGAAATTATTTACTTGTCAGAAAACAGAATATCACCGTTGGTAAAAGATACCCG
GCAGAGTTATGCAAATAGTTCCTCTTTTCAACGTCATATCCGGAAACGACGCTCAACAG
ATTTTGAGTTTGACCCACATTCGAACTTTTATCATTTCACCCGTCCTTTAATAAAGCCACA
ATGTGCTGCTTATGGAAAAGCCTTAGATTTAAGCCTCAACAGTATTTTCTTCATTGGGCC
AAACCAATTTGAAAATCTTCCTGACATTGCCTGTTTAAATCTGTCTGCAAATAGCAATGC
TCAAGTGTTAAGTGGAACTGAATTTTCAGCCATTCCTCATGTCAAATATTTGGATTTGAC
AAACAATAGACTAGACTTTGATAATGCTAGTGCTCTTACTGAATTGTCCGACTTGGAAGT
TCTAGATCTCAGCTATAATTCACACTATTTCAGAATAGCAGGCGTAACACATCATCTAGA
ATTTATTCAAAATTTCACAAATCTAAAAGTTTTAAACTTGAGCCACAACAACATTTATACTT
TAACAGATAAGTATAACCTGGAAAGCAAGTCCCTGGTAGAATTAGTTTTCAGTGGCAAT
CGCCTTGACATTTTGTGGAATGATGATGACAACAGGTATATCTCCATTTTCAAAGGTCTC
AAGAATCTGACACGTCTGGATTTATCCCTTAATAGGCTGAAGCACATCCCAAATGAAGC
ATTCCTTAATTTGCCAGCGAGTCTCACTGAACTACATATAAATGATAATATGTTAAAGTTT
TTTAACTGGACATTACTCCAGCAGTTTCCTCGTCTCGAGTTGCTTGACTTACGTGGAAAC
AAACTACTCTTTTTAACTGATAGCCTATCTGACTTTACATCTTCCCTTCGGACACTGCTG
CTGAGTCATAACAGGATTTCCCACCTACCCTCTGGCTTTCTTTCTGAAGTCAGTAGTCTG
AAGCACCTCGATTTAAGTTCCAATCTGCTAAAAACAATCAACAAATCCGCACTTGAAACT
AAGACCACCACCAAATTATCTATGTTGGAACTACACGGAAACCCCTTTGAATGCACCTG
TGACATTGGAGATTTCCGAAGATGGATGGATGAACATCTGAATGTCAAAATTCCCAGAC
TGGTAGATGTCATTTGTGCCAGTCCTGGGGATCAAAGAGGGAAGAGTATTGTGAGTCT
GGAGCTAACAACTTGTGTTTCAGATGTCACTGCAGTGATATTATTTTTCTTCACGTTCTTT
ATCACCACCATGGTTATGTTGGCTGCCCTGGCTCACCATTTGTTTTACTGGGATGTTTG
GTTTATATATAATGTGTGTTTAGCTAAGGTAAAAGGCTACAGGTCTCTTTCCACATCCCA
AACTTTCTATGATGCTTACATTTCTTATGACACCAAAGATGCCTCTGTTACTGACTGGGT
GATAAATGAGCTGCGCTACCACCTTGAAGAGAGCCGAGACAAAAACGTTCTCCTTTGTC
TAGAGGAGAGGGATTGGGATCCGGGATTGGCCATCATCGACAACCTCATGCAGAGCAT
CAACCAAAGCAAGAAAACAGTATTTGTTTTAACCAAAAAATATGCAAAAAGCTGGAACTT
TAAAACAGCTTTTTACTTGGCTTTGCAGAGGCTAATGGATGAGAACATGGATGTGATTAT
ATTTATCCTGCTGGAGCCAGTGTTACAGCATTCTCAGTATTTGAGGCTACGGCAGCGGA
TCTGTAAGAGCTCCATCCTCCAGTGGCCTGACAACCCGAAGGCAGAAGGCTTGTTTTG
GCAAACTCTGAGAAATGTGGTCTTGACTGAAAATGATTCACGGTATAACAATATGTATGT
CGATTCCATTAAGCAATACTAA
TLR10 coding sequence

SEQ ID NO: 17

ATGAGACTCATCAGAAACATTTACATATTTTGTAGTATTGTTATGACAGCAGAGGGTGAT
GCTCCAGAGCTGCCAGAAGAAAGGGAACTGATGACCAACTGCTCCAACATGTCTCTAA
GAAAGGTTCCCGCAGACTTGACCCCAGCCACAACGACACTGGATTTATCCTATAACCTC
CTTTTTCAACTCCAGAGTTCAGATTTTCATTCTGTCTCCAAACTGAGAGTTTTGATTCTAT
GCCATAACAGAATTCAACAGCTGGATCTCAAAACCTTTGAATTCAACAAGGAGTTAAGAT
ATTTAGATTTGTCTAATAACAGACTGAAGAGTGTAACTTGGTATTTACTGGCAGGTCTCA
GGTATTTAGATCTTTCTTTTAATGACTTTGACACCATGCCTATCTGTGAGGAAGCTGGCA
ACATGTCACACCTGGAAATCCTAGGTTTGAGTGGGGCAAAAATACAAAAATCAGATTTC
CAGAAAATTGCTCATCTGCATCTAAATACTGTCTTCTTAGGATTCAGAACTCTTCCTCATT
ATGAAGAAGGTAGCCTGCCCATCTTAAACACAACAAAACTGCACATTGTTTTACCAATG
GACACAAATTTCTGGGTTCTTTTGCGTGATGGAATCAAGACTTCAAAAATATTAGAAATG
ACAAATATAGATGGCAAAAGCCAATTTGTAAGTTATGAAATGCAACGAAATCTTAGTTTA
GAAAATGCTAAGACATCGGTTCTATTGCTTAATAAAGTTGATTTACTCTGGGACGACCTT
TTCCTTATCTTACAATTTGTTTGGCATACATCAGTGGAACACTTTCAGATCCGAAATGTG
ACTTTTGGTGGTAAGGCTTATCTTGACCACAATTCATTTGACTACTCAAATACTGTAATG
AGAACTATAAAATTGGAGCATGTACATTTCAGAGTGTTTTACATTCAACAGGATAAAATC
TATTTGCTTTTGACCAAAATGGACATAGAAAACCTGACAATATCAAATGCACAAATGCCA
CACATGCTTTTCCCGAATTATCCTACGAAATTCCAATATTTAAATTTTGCCAATAATATCT
TAACAGACGAGTTGTTTAAAAGAACTATCCAACTGCCTCACTTGAAAACTCTCATTTTGA
ATGGCAATAAACTGGAGACACTTTCTTTAGTAAGTTGCTTTGCTAACAACACACCCTTGG
AACACTTGGATCTGAGTCAAAATCTATTACAACATAAAAATGATGAAAATTGCTCATGGC
CAGAAACTGTGGTCAATATGAATCTGTCATACAATAAATTGTCTGATTCTGTCTTCAGGT
GCTTGCCCAAAAGTATTCAAATACTTGACCTAAATAATAACCAAATCCAAACTGTACCTA
AAGAGACTATTCATCTGATGGCCTTACGAGAACTAAATATTGCATTTAATTTTCTAACTGA
TCTCCCTGGATGCAGTCATTTCAGTAGACTTTCAGTTCTGAACATTGAAATGAACTTCAT
TCTCAGCCCATCTCTGGATTTTGTTCAGAGCTGCCAGGAAGTTAAAACTCTAAATGCGG
GAAGAAATCCATTCCGGTGTACCTGTGAATTAAAAAATTTCATTCAGCTTGAAACATATT
CAGAGGTCATGATGGTTGGATGGTCAGATTCATACACCTGTGAATACCCTTTAAACCTA
AGGGGAACTAGGTTAAAAGACGTTCATCTCCACGAATTATCTTGCAACACAGCTCTGTT
GATTGTCACCATTGTGGTTATTATGCTAGTTCTGGGGTTGGCTGTGGCCTTCTGCTGTC
TCCACTTTGATCTGCCCTGGTATCTCAGGATGCTAGGTCAATGCACACAAACATGGCAC
AGGGTTAGGAAAACAACCCAAGAACAACTCAAGAGAAATGTCCGATTCCACGCATTTAT
TTCATACAGTGAACATGATTCTCTGTGGGTGAAGAATGAATTGATCCCCAATCTAGAGAA
GGAAGATGGTTCTATCTTGATTTGCCTTTATGAAAGCTACTTTGACCCTGGCAAAAGCAT
TAGTGAAAATATTGTAAGCTTCATTGAGAAAAGCTATAAGTCCATCTTTGTTTTGTCTCCC
AACTTTGTCCAGAATGAGTGGTGCCATTATGAATTCTACTTTGCCCACCACAATCTCTTC
CATGAAAATTCTGATCATATAATTCTTATCTTACTGGAACCCATTCCATTCTATTGCATTC
CCACCAGGTATCATAAACTGAAAGCTCTCCTGGAAAAAAAAGCATACTTGGAATGGCCC
AAGGATAGGCGTAAATGTGGGCTTTTCTGGGCAAACCTTCGAGCTGCTATTAATGTTAA
TGTATTAGCCACCAGAGAAATGTATGAACTGCAGACATTCACAGAGTTAAATGAAGAGT
CTCGAGGTTCTACAATCTCTCTGATGAGAACAGATTGTCTATAA
SFRS8 coding sequence

SEQ ID NO: 18

ATTTTGTGGCCCGCTATGGCGGCGGTGTTGAGGTTGGGTACGGGATGCGGGGTCTTTG
ACTGAAGGGGTAGGCCAAGTGGAGGTATCAGGGACGTCGCGCGGCACAGAAGAG-
GACCAGCCTGGACGCCGGGGACGCTGTCATG-
TACGGCGCGAGCGGGGGCCGCGCCAAACCCGAGAGGAAAAGCGGCGCGAAGGAG-
GAGGCCGGGCCAGGCGGTGCCGGCGGTGGGGGCAGCCGAGTGGAGCTCTTGGTTTT
CGGCTATGCCTGCAAGCTGTTCCGGGACGACGAGCGGGCCCTGGCTCAGGAACAGG-
GACAGCACCTCATCCCCTGGATGGGGGACCACAAGATCCTCATCGACAGATATGATG-
GACGTGGTCACCTGCATGACCTTTCTGAGTACGATGCTGAGTATTCCACGTGGAACA-
GAGATTATCAGCTGTCTGAAGAGGAGGCGCGAATAGAGGCCCTGTGTGATGAAGA-
GAGGTATTTAGCCTTGCATACGGACTTGCTTGAGGAGGAGGCAAGGCAAGAGGAA-
GAATACAAGCGATTGAGTGAAGCACTAGCAGAGGATGGGAGCTA-
CAATGCCGTGGGGTTCACTTACGGTAGCGACTATTACGACCCGTCAGAGCCGACG-
GAGGAGGAGGAGCCTTCCAAACAGAGAGAAAAAAATGAGGCCGAAAATTTAGAG-
GAAAATGAAGAGCCCTTCGTTGCCCCCTTAGGATT-
GAGCGTCCCGTCTGACGTGGAGTTGCCACCAACCGCTAAAATGCACGCCATCATC-
GAGCGCACGGCCAGCTTCGTGTGCAGGCAGGGAGCACAGTTTGAGAT-
CATGCTGAAGGCCAAGCAGGCCCGGAACTCCCAGTTTGACTTTCTGCGCTTCGAC-
CACTACCTCAACCCCTACTATAAGTTCATCCAGAAAGCCATGAAAGAGGGACGCTA-
CACTGTCCTGGCAGAAAACAAAAGTGACGAGAAAAAAAAATCAGGAGTCAGCTCTGA-
CAATGAAGATGATGATGATGAAGAAGATGGGAAT-
TACCTTCATCCCTCTCTCTTTGCCTCCAAGAAGTGTAACCGCCTTGAAGAGCTGAT-
GAAGCCCTTGAAGGTAGTGGACCCAGATCATCCCCTCGCAGCACTTGTTCGTAAGG-
CACAGGCTGACAGTTCCACTCCCACCCCACACAACGCA-
GACGGTGCGCCTGTGCAGCCCTCCCAGGTGGAGTACACGGCA-
GACTCGACCGTGGCAGCCATGTATTACAGCTACTACATGCTACCGGACGGCACT-
TACTGCCTGGCGCCGCCCCCTCCCGGAATCGACGTGACTACTTACTACAG-
CACCCTTCCTGCTGGCGTGACCGTGTCTAACTCCCCTGGAGTGACGAC-
CACCGCCCCACCACCTCCTGGGACCACACCACTACCGCCCCCAACCACAGCAGAGAC-
TAGCAGCGGGGCCACCTCCACAACCACCACCA-
CAAGTGCACTTGCCCCCGTGGCCGCCAT-
CATCCCCCCGCCCCCCGACGTCCAGCCCGTGATTGACAAGCTGGCCGAG-
TATGTCGCCAGGAACGGCCTGAAGTTCGAGACCAGTGTTCGTGCCAAGAATGAT-
CAAAGATTTGAGTTCCTGCAGCCGTGGCACCAGTATAATGCTTATTATGAGTTTAA-
GAAGCAGTTCTTCCTCCAGAAAGAAGGGGGCGATAGCATGCAGGCTGTGTCTGCAC-
CAGAAGAGGCTCCCACAGACTCTGCTCCCGAGAAGCCAAGTGATGCTGGGGAG-
GATGGCGCGCCTGAAGACGCAGCCGAGGTGGGAG-
CACGGGCAGGCTCAGGCGGGAAGAAGGAGGCATCGTCCAGTAA-
GACCGTCCCGGACGGGAAGCTGGTGAAAGCTTCCTTTGCTCCAATAAGCTTTGCAAT-
CAAGGCCAAAGAAAATGATCTGCTTCCCCTGGAAAAAAATCGTGTTAAGCTAGATGAT-
GACAGTGATGATGATGAAGAAAGCAAAGAAGGCCAAGAAAGTTCTAG-
TAGTGCTGCAAACACTAACCCAGCAGTTGCCCCACCCTGTGTAGTTGTTGAGGAGAA-
GAAGCCTCAACTTACCCAGGAGGAGCTAGAAGCAAAGCAAGCAAAGCAAAAGCTGGAA
CAAAAGCTGGAA-
GATCGCCTCGCAGCTGCTGCCCGGGAAAAGCTGGCCCAGGCGTCTAAGGAGT-
CAAAAGAGAAACAGCTTCAAGCAGAACGTAAAAGGAAAGCGGCGTTATTTTTACA-
GACCCTCAAAAATCCTCTGCCGGAAGCAGAAGCTGGGAAAATTGAGGA-
GAGTCCTTTCAGTGTCGAGGAATCCAGCACTACGCCCTGCCCTCTACTGACTGGAGG-
CAGGCCTCTGCCTACTTTAGAAGTTAAACCACCCGATAGGCCTTCGAGCAAAAGCAAA-
GATCCACCGAGAGAAGAAGAGAAAGAAAAGAAAAAGAAAAAGCACAAAAAAA-
GATCTCGAACAAGATCACGTTCTCCCAAGTACCATTCGTCATCCAAGTCCAGGTCTA-
GATCACACTCAAAAGCAAAGCATTCTCTTCCCAGTGCCTATCGGA-
CAGTGCGGCGGTCGAGGTCCCGCTCCCGGTCCCCTCGGAGGA-
GAGCCCACTCCCCTGAGAGACGGAGGGAAGAGAG-
GAGTGTGCCCACTGCCTACCGCGTGAGCCGCAGCCCTGGGGCCAGCAG-
GAAGCGGACCCGCTCCAGAAGTCCCCACGAGAAGAAGAAGAA-
GAGGCGGTCCCGGTCGCGGACCAAGTCCAAGGCCAGGTCTCAGTCGGTGTCACC-
CAGCAAGCAGGCAGCGCCCCGGCCCGCGGCCCCCGCGGCCCACTCGGCGCACT-
CAGCCAGCGTCTCCCCTGTGGAGAGTCGGGGCTCCAGCCAG-
GAGCGCTCCAGGGGAGTCTCTCAGGAAAAAGAAGCCCAGATCTCTTCAG-
CAATCGTTTCTTCCGTGCAGAGCAAAATCACTCAGGATCTCATGGCCAAAGTCAGAGC-
GATGCTTGCAGCTTCCAAAAACCTGCAAACCAGCGCTTCCTGA-
GACGGGGCCAGCGGAGGCA-
GAGCCGGGAGGCTGCGTGGGCTTCTGGGCAGGCTCACGCAGACGCCGGCCACAC-
CATCCACCTGGCCGCCTCCATGGACCCTTGGTGGCTTTTGTAAATTAATTTTTGATGA-
CATTTTGAGTTTTAAGATTTCTGACCAGCAGTCTCTTACCTGTATATTTGTAAATATAT-
CATGTTTCTGTGAAAATGTATTATGAAATAAAATGGGAGGAAACACCTTTTCTAGCTAG

1. A method for determining a predisposition to an immune-related disease or condition in a subject comprising determining in a biological sample isolated from said subject two or more polymorphisms in one or more immune related genes selected from the SFRS8, SLAMF1, CD86, TLR7, TLR8, TLR10, IL2, CD83 and/or HRH1 genes and/or in chromosome regions containing said genes, or in a translational or transcriptional products of said genes or in translational or transcriptional products of said chromosome regions.

2. The method according to claim 1, wherein the two or more polymorphisms are determined in one gene selected from the SFRS8, SLAMF1, CD86, TLR7, TLR8, TLR10, IL2, CD83 and/or HRH1 genes or in a chromosome region containing said gene.

3-57. (canceled)

58. The method according to claim 1, wherein at least one of the polymorphisms is the single nucleotide polymorphism (SNP).

59. (canceled)

60. The method according to claim 58, wherein the SNP(s) is(are) selected from the group consisting of the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233 or rs1379049.

61-63. (canceled)

64. The method according to claim 60, wherein the SNP(s) is(are) present in
a nucleotide sequence selected from SEQ ID NOs: 1-8 or 9,
a nucleotide sequence having at least 90% sequence identity with a sequence of (i), or a fragment thereof, or
a nucleotide sequence being complementary to any of the sequences of (i) or (ii).

65-67. (canceled)

68. A method for determining a predisposition to an immune-related disease in a subject comprising determining in a biological sample isolated from said subject a polymorphism in the CD86 gene and/or a region of the human chromosome 3q being in linkage disequilibrium with the CD86 gene or in a translational or transcriptional product from said gene or said chromosome region, said polymorphism being indicative of said predisposition.

69. The method according to claim 68, wherein the chromosome region contains the CD86 gene.

70. The method of claim 69, wherein the polymorphism is present in a nucleotide sequence of the CD86 gene, or a sequence being complementary to the sequence of said gene.

71-75. (canceled)

76. A method for determining a predisposition for an immune-related disease in a subject comprising determining in a biological sample isolated from said subject a polymorphism in the SLAMF1 gene and/or in a part of the human chromosome 1q being in linkage disequilibrium with the SLAMF1 gene or in a translational or transcriptional product from said part, said polymorphism being indicative of said predisposition.

77. The method according to claim 76, wherein the chromosome region contains the SLAMF1 gene.

78. The method according to claim 77, wherein the polymorphism is determined in a non-coding region of the SLAMF1 gene such as an intron or a region controlling expression of the SLAMF1 gene.

79-86. (canceled)

87. A method for determining a predisposition to an immune-related disease in a subject comprising determining in a biological sample isolated from said subject a polymorphism in the TLR7 gene and/or in a region of the human chromosome Xp22 being in linkage disequilibrium with the TLR7 gene, or in a translational or transcriptional product from said gene or said chromosome region, said polymorphism being indicative of said predisposition.

88. The method according to claim 87, wherein the chromosome region contains the TLR7 gene.

89-95. (canceled)

96. A method for determining a predisposition to an immune-related disease in a subject comprising determining in a biological sample isolated from said subject a polymorphism in the TLR10 gene and/or in a region of the human chromosome p4 being in linkage disequilibrium with the TLR10 gene, or in a translational or transcriptional product from said gene or said chromosome region said polymorphism being indicative of said predisposition.

97. (canceled)

98. The method according to claim 96, wherein the polymorphism is a SNP.

99. The method according to claim 98, wherein the SNP is determined in a non-coding region of the TRL10 gene such as an intron or a region controlling expression of the TRL10 gene.

100. (canceled)

101. The method according to claim 99, wherein the SNP is selected form the SNPs having refSNP nos. rs11466642, rs11466645, rs1109696, rs11096955, rs11466655 or rs11466657.

102-108. (canceled)

109. A method for determining a predisposition to an immune-related disease in a subject comprising determining in a biological sample isolated from said subject a polymorphism in the TLR8 gene and/or in a region of the human chromosome p22 being in linkage disequilibrium with the TLR8 gene, or in a translational or transcriptional product from said gene or chromosome region, said polymorphism being indicative of said predisposition.

110-111. (canceled)

112. A method for determining a predisposition for an immune-related disease in a subject comprising determining in a biological sample isolated from said subject a polymorphism in the SFRS8 gene or in a part of the human chromosome 12q being in linkage disequilibrium with the SFRS8 gene, or in a translational or transcriptional product from said gene or said chromosome part, said polymorphism being indicative of said predisposition.

113-114. (canceled)

115. A method for determining a predisposition for an immune-related disease in a subject comprising determining in a biological sample isolated from said subject a polymorphism in the HRH1 gene and/or in a region of the human chromosome 3q being in linkage disequilibrium with the HRH1 gene or in a translational or transcriptional product from said gene or said chromosome region, said polymorphism being indicative of said predisposition.

116-117. (canceled)

118. A method for determining a predisposition for an immune-related disease in a subject comprising determining in a biological sample isolated from said subject a polymorphism in the IL2 gene and/or in a part of the human chromosome 4q being in linkage disequilibrium with the IL2 gene or in a translational or transcriptional product from said gene or said chromosome part, said polymorphism being indicative of said predisposition.

119-120. (canceled)

121. A method for determining a predisposition for an immune-related disease in a subject comprising determining in a biological sample isolated from said subject a polymorphism in the CD83 gene and/or in a part of the human chromosome 6p being in linkage disequilibrium with the CD83 gene or in a translational or transcriptional product from said gene or said chromosome part, said polymorphism being indicative of said predisposition.

122. The method according to claim 121, wherein the polymorphism is a SNP.

123. The method according to claim 122, wherein the SNP is prom2 SNP.

124-129. (canceled)

130. The method according to claim 1, wherein the immune-related disease is selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema.

131-141. (canceled)

142. A method for determining a predisposition for not having an immune-related disease in a subject comprising determining in a biological sample isolated from said subject the protective allele of a SNP(s) according to claim 60.

143. An isolated oligonucleotide comprising at least 10 contiguous nucleotides being 100% identical to a subsequence of a gene selected from of the SFRS8, SLAMF1, CD86, CD83, IL2, HRH1, TLR7, TLR8, or TLR10 genes, comprising or adjacent to a polymorphism or mutation being correlated to an immune-related disease such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema.

144-147. (canceled)

148. The isolated oligonucleotide according to claim 143, said oligonucleotide being selected from the nucleic acid sequences identified as SEQ ID NO: 19-126.

149. The isolated oligonucleotide according to claim 143, wherein the nucleotides are selected from RNA, DNA, LNA, PNA monomers or chemically modified nucleotides capable of hybridising to a target nucleic acid sequence.

150. A kit for predicting the risk of a subject of developing an immune related disease comprising at least two oligonucleotides as defined in claim 143.

151. The kit according to claim 150, wherein the at least two oligonucleotides are the amplification primers or probes for determining a polymorphism associated with a predisposition for an immune-related disease as defined in any of the preceding claims.

152-153. (canceled)

154. A variant protein, wherein the amino acid substitution is Val residue substituting Ile residue at position 179 of B7-2 protein, Pro residue substituting Thr residue at position 333 of SLAM protein, Phe residue substituting Leu residue at position 11 of SLAM protein, Pro residue substituting Thr residue at position 333 of SLAM protein, Thr substituting Leu at position 473 of TLR10 protein, Asp substituting Gly at position 38 of TLR10 protein, H is substituting Asp at position 241 of TLR10 protein, or Leu substituting Ile at position 369 of TLR10 protein said protein being indicative of a predisposition to an immune-related disease selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema.

155. An antibody capable of selectively binding to a variant protein of claim 154 to an epitope comprising a residue defined in claim 154.

156. A method for treatment of an immune related disease selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema in a subject being diagnosed as having a predisposition to said disease by using a method according to claim 1, comprising administering to said subject a therapeutically effective amount of a gene therapy vector, said gene therapy vector comprising the protective allele of an SNP, wherein the SNP(s) is(are) selected from the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs 1096955, rs 1096956, rs 1096957, rs11466645, rs11466642, rs2407992, rs755437, rs378288, rs1051219, rs1051233 or rs1379049.

157. A vector comprising a nucleic acid sequence selected from the nucleic acid sequences identified as SEQ ID NO: 10-18, wherein said nucleic sequence comprising a polymorphism associated with a predisposition to an immune related disease selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema, said predisposition being determined by using a method according to claim 1, wherein said nucleic acid sequence is operably linked to a promoter sequence capable of directing the expression of a mutant protein encoded by said sequence.

158. A host cell transformed or transfected with the vector of claim 157.

159. Use of a compound capable of decreasing or modulating the co-stimulatory signal in T-cell activation for the preparation of a medicament for the treatment of allergy related diseases in a subject being diagnosed as having a predisposition to an immune related disease by a method according to claim 1.

160. The use according to claim 159, wherein the immune related disease is selected from Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema.

161-162. (canceled)

163. A method of vaccination of a subject having a predisposition to an immune related disease determined by a method according to claim 1, said method comprising immunising said subjects with a therapeutically effective amount of a specific allergen.

164. A method for determining a protection against an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema, in a subject comprising determining in a biological sample isolated from said subject the protective allele of an SNP associated with a predisposition of an individual to said disease, wherein said SNP is selected from the group consisting of the SNPs identified as rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233 and rs1379049.

165. A gene therapy vector comprising
a. a DNA sequence selected from the sequences identified as SEQ ID NO 1-9, or a fragment thereof, or
b. a DNA sequence selected from the sequences identified as SEQ ID NOs: 10-18, or a fragment of said DNA sequence.

166. The gene therapy vector according to claim 165, wherein the DNA sequence or a fragment thereof comprises the protective allele of an SNP selected from the group consisting of the SNPs identified as rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233, and rs1379049.

167. A method of treatment of a subject having the predisposition to an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings, Angio oedema, said method comprising administering to said subject a therapeutically effective amount of a gene therapy vector as defined in claim 165.

168. A compound capable of
i) modulating expression of an immune related gene selected from the genes according to claim 1, said gene comprising a SNP selected from the group consisting of the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233 or rs1379049, wherein said compound is selected from an isolated antisense nucleotide sequence or an nucleotide sequence complementary to the regulatory region of said gene, said nucleotide sequence being capable of forming triple helix structures that prevent transcription of said gene, and/or
ii) modulating activity of a transcriptional product of an immune related gene selected from the genes according to claim 1, said gene comprising a SNP selected from the group consisting of the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233 or rs1379049, wherein the transcriptional product being selected from a nucleic acid sequence identified as SEQ ID NO: 10-17 or 18, or a fragment thereof, a nucleic acid sequence having at least 90% identity with a nucleic sequence of, or a nucleic acid sequence being complementary to any of the sequences of, or a fragment thereof, said nucleic acid sequences comprising the polymorphism(s) corresponding to polymorphism(s) of a genomic sequence identified as SEQ ID NO: 1-8 or 9, which is(are) indicative of a predisposition to an immune related disease, wherein said compound is selected from an isolated antisense sequence or a ribozyme molecule, and/or
iii) modulating activity of a translational product of an immune related gene selected from the genes according to claim 1, said gene comprising a SNP selected from the group consisting of the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs11096955, rs11096956, rs11096957, rs11466645, rs11466642, rs2407992, rs755437, rs378288, rs1051219, rs1051233 or rs1379049 wherein said translational product being a polypeptide having the amino acid sequence identical to an amino acid sequence selected from the sequences identified as Swiss-prot Ass. No: NP 003028 (SLAMF1), NP 999387 (CD86), NP 004224 (CD83), NP 000852 (HRH1), NP 000577 (IL2), NP 057646 (TLR7), NP 619542 (TLR8), NP 112218 (TLR10), NP 004583 (SFRS8), said polypeptide comprising a polymorphism(s) corresponding to the polymorphism(s) of a nucleic acid sequence(s) encoding said polypeptide(s) or a fragment(s) thereof comprising said polymorphism(s), or a polypeptide having the amino acid sequence having at least 90% identity with said sequence, or a fragment thereof, wherein a nucleic acid sequence encoding said polypeptide is selected from SEQ ID NOs: 1-9 or 10-18, or a nucleic acid sequence complementary thereof, or is a fragment of any of said nucleic acid sequences, wherein said compound is selected from an antibody molecule against said translational product, or a molecule capable of interfering with biological activity of said translational product.

169. (canceled)

170. A pharmaceutical composition for the treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said composition comprising a compound according to claim 168.

171. A method of treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, comprising administering a compound according to claim 168.

172. A method of screening for a candidate compound for therapeutic treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said method comprising an in vitro or an in vivo model system comprising a gene according to claim 1 or a product of said gene, said product being a transcriptional product selected from a nucleic acid sequence identified as SEQ ID NO: 10-17 or 18, or a fragment thereof, a nucleic acid sequence having at least 90% identity with said nucleic sequence of, or a nucleic acid sequence being complementary to any of the sequences of, or a fragment thereof, said nucleic acid sequences comprising the polymorphism(s) corresponding to polymorphism(s) of a genomic sequence identified as SEQ ID NO: 1-8 or 9, which is(are) indicative of a predisposition to an immune related disease, or a translational product of the gene having the amino acid sequence identical to an amino acid sequence selected from the sequences identified as Swiss-prot Ass. No: NP 003028 (SLAMF1), NP 999387 (CD86), NP 004224 (CD83), NP 000852 (HRH1), NP 000577 (IL2), NP 057646 (TLR7), NP 619542 (TLR8), NP 112218 (TLR10), NP 004583 (SFRS8), said polypeptide comprising a polymorphism(s) corresponding to the polymorphism(s) of a nucleic acid sequence(s) encoding said polypeptide(s) or a fragment(s) thereof comprising said polymorphism(s), or a polypeptide having the amino acid sequence having at least 90% identity with said sequence, or a fragment thereof, wherein a nucleic acid sequence encoding said polypeptide is selected from SEQ ID NOs: 1-9 or 10-18, or a nucleic acid sequence complementary thereof, or is a fragment of any of said nucleic acid sequences.

173. (canceled)

174. A method for prognosis of the likelihood of development of an immune related disease comprising determining a polymorphism in a gene selected from the genes according to claim 1, said polymorphism being an SNP selected from the group consisting of the SNPs having refSNP IDs: rs3796504, rs2295619, rs12076998, rs1000807, rs2295613, rs179008, rs5743781, rs864058, rs5741883, rs3764879, rs3764880, rs5744077, rs2159377, rs11466657, rs11466655, rs 1096955, rs 1096956, rs 1096957, rs11466645, rs11466642, rs2407992, rs755437, rs378288, rs1051219, rs1051233 or rs1379049.

175. (canceled)

176. A method of predicting the likelihood of a subject to respond to a therapeutic treatment of an immune related disease, such as Asthma, bronchial hyperresponsiveness, Rhinitis/hayfever, Conjunctivitis/rhino conjuntivitis, Atopic dermatitis/eczema, systemic anaphylaxis, contact dermatitis, Urticaria, hypersensitivity reactions types I-IV, Oral allergy syndrome, Allergic Gastrointestinal reactions, Systemic reactions after insect stings or Angio oedema, said method comprising determining the genotype of said subject in the SFRS8, CD83, SLAMF1, CD86, HRH1, IL2, TLR7, TLR8 and/or TLR10 gene and/or in the chromosome areas comprising the SFRS8, CD83, SLAMF1, CD86, HRH1, IL2, TLR7, TLR8 and/or TLR10 gene.
